(19) United States
(12) Patent Application Publication
(10) Pub. No.: US 2022/0112194 A1
(43) Pub. Date: Apr. 14, 2022

(54) COMPOUNDS TARGETING PRMT5

(71) Applicant: ALIGOS THERAPEUTICS, INC., South San Francisco, CA (US)

(72) Inventors: Koen Vandyck, Paal (BE); Pierre Jean-Marie Bernard Raboisson, Wavre (BE); Jerome Deval, Pacifica, CA (US); Leonid Beigelman, San Mateo, CA (US); David McGowan, Brussels (BE); Yannick Debing, Bilzen (BE); Francois Gonzalvez, Antwerp (BE)

(21) Appl. No.: 17/217,193

(22) Filed: Mar. 30, 2021

Related U.S. Application Data
(60) Provisional application No. 63/003,723, filed on Apr. 1, 2020.
(60) Provisional application No. 63/077,274, filed on Sep. 11, 2020.

(51) Int. Cl. C07D 487/04; C07D 519/00; C07D 471/04; A61P 35/00

(52) U.S. Cl. CPC C07D 487/04; A61P 35/00; C07D 471/04; C07D 519/00

(57) ABSTRACT

Provided herein are compounds of Formula (I), or pharmaceutically acceptable salts thereof, pharmaceutical compositions that include a compound described herein (including pharmaceutically acceptable salts of a compound described herein) and methods of synthesizing the same. Also provided herein are methods of treating diseases and/or conditions with a compound of Formula (I), or a pharmaceutically acceptable salt thereof.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application Nos. 63/003,723, filed Apr. 1, 2020 and 63/077,274, filed Sep. 11, 2020.

### BACKGROUND

### Field

The present application relates to the fields of chemistry, biochemistry and medicine. Disclosed herein are compounds of Formula (I), or pharmaceutically acceptable salt thereof, pharmaceutical compositions that include a compound described herein (including pharmaceutically acceptable salts of a compound described herein) and methods of synthesizing the same. Also disclosed herein are methods of treating diseases and/or conditions with a compound of Formula (I), or a pharmaceutically acceptable salt thereof.

### Description

In mammals, there are nine enzymes in the seven-β-strand family of protein arginine methyltransferases (PRMTs), designated PRMT1-9. These PRMTs are further divided into three types based on the different methylarginine derivatives they produce: Type I PRMTs (PRMT1-4, 6, and 8) catalyze the production of monomethylarginine (MMA) and asymmetric dimethylarginine (ADMA); Type II PRMTs (PRMT5 and 9) catalyze MMA and symmetric dimethylarginine (SDMA) production; and Type III enzymes (PRMT7) catalyze only the production of MMA residues.

### SUMMARY

Some embodiments disclosed herein relate to a compound of Formula (I), or a pharmaceutically acceptable salt thereof.

Some embodiments disclosed herein relate to a pharmaceutical composition that can contain an effective amount of a compound of Formula (I), or a pharmaceutically acceptable salt thereof.

These are other embodiments are described in greater detail below.

### DETAILED DESCRIPTION

PRMT5 is a Type II protein arginine methyltransferase that catalyzes SDMA modification in histones and non-histone substrates, including three subunits of the Survival of Motor Neuron (SMN) complex (SmB, SmD1 and SmD3). These proteins are essential components of the spliceosome machinery (See Friesen et al., Molecular and Cellular Biology (2001) 21(24):8289-8300; Matera et al., Nature Reviews Molecular Cell Biology (2014) 14:108-121; and Meister et al., Current Biology (2001) 11(24): 1990-1994), and PRMT5 depletion triggers aberrant splicing in the adult hematopoietic compartment (Bezzi et al., Genes & Development (2013) 27:1903-1916; Koh et al., Nature (2015) 523:96-100; and Liu et al., J. Clin. Invest. (2015) 125(9):3532-3544).

PRMT5 is overexpressed in a variety of human cancers, including several hematological malignancies such as lymphoma and leukemia (Yang et al., Nature Reviews Cancer (2013) 13:37-50 and Chung et al., J. Biol. Chem. (2013) 288(49):35534-35547), as well as liver cancer (Jiang et al., Cancer Medicine (2018) 7(3):869-882), lung cancer (Wei et al., Cancer Science (2012) 103(9): 1640-1650), breast cancer (Powers et al., Cancer Research (2011) 71(16):5579-5587), and colorectal cancer (Cho et al., The EMBO Journal (2012) 31:1785-1797). Enhanced PRMT5 expression correlates with reduced overall survival and higher recurrence rates for patients with hepatocellular carcinoma (HCC) (Jiang et al., Cancer Medicine (2018) 7(3):869-882). Knocking down PRMT5 expression with shRNA can prevent cell proliferation and colony formation in Huh-7 and SK-Hep1 HCC cells. In a mouse xenograph model for HCC, this approach can result in tumor regression.

Inhibition of PRMT5 has been shown to result in anti-tumor activity in lymphomas (Chan-Penebre et al., Nature Chemical Biology (2015) 11:432-437), MLL-rearranged acute leukemia models (Kaushik et al., Leukemia (2018) 32:499-509), and several other types of leukemia in vitro (Tarighat et al., Leukemia (2016) 30:789-799). In addition, cells lacking MTAP, a critical enzyme in the methionine salvage pathway that is deleted in approximately 15% of all human cancers, can be more sensitive to PRMT5 depletion than MTAP wild type cells (Kryukov et al., Science (2016) 351(6278): 1214-1218; Marjon et al., Cell Report (2016) 15(3):574-587; and Mavrakis et al., Science (2016) 351(6278): 1208-1213). Small molecule inhibitors of PRMT5 have shown preferential impairment of cell viability for MTAP-null cancer cell lines compared with isogenic MTAP-expressing counterparts, making PRMT5 a potential vulnerability across multiple cancer lineages.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents (for example, a group may be substituted with 1, 2, 3, 4 or 5 substituents). If no substituents are indicated, it is meant that the indicated “optionally substituted” or “substituted” group may be substituted with one or more group(s) individually and independently selected from deuterium, alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. The length of an alkenyl can vary. For example, the alkenyl can be a C₂₋₄ alkenyl, C₂₋₆ alkenyl or C₂₋₈ alkenyl. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. The length of an alkynyl can vary. For example, the alkynyl can be a C₂₋₄ alkynyl, C₂₋₆ alkynyl or C₂₋₈ alkynyl. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to a monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The number of atoms in the ring(s) of a heterocyclyl group can vary. For example, the heterocyclyl group can contain 4 to 14 atoms in the ring(s), 5 to 10 atoms in the ring(s) or 5 to 6 atoms in the ring(s). The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl groups may be unsubstituted or substituted. Examples of such “heterocyclyl groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine A-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiomorpholine, thiomorpholine sulfoxide, thiomorpholine sulfone and their benzo-fused analogs (e.g., benzimidazolidinone, tetrahydroquinoline and 3,4-methylenedioxyphenyl).

As used herein, “aryl(alkyl)” refer to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl) and naphthyl(alkyl).

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl) and their benzo-fused analogs.

A “(heterocyclyl)alkyl” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, aryl(alkyl), heteroaryl(alkyl) or heterocyclyl(alkyl) is defined herein. In some instances, an alkoxy can be —O(an unsubstituted C₁₋₄ alkyl). A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen an alkyl, an alkenyl, an alkynyl, a cycloalkyl, a cycloalkenyl, aryl, heteroaryl, heterocyclyl, aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to a O-alkyl group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

A “sulfenyl” group refers to an “—SR” group in which R can be hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} is hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-amido may be substituted or unsubstituted.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound or composition, the term “comprising” means that the compound or composition includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomeric ally enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

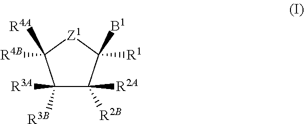

wherein: B¹ can be an optionally substituted

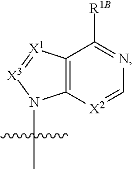

an optionally substituted

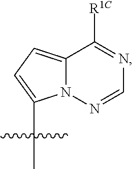

an optionally substituted

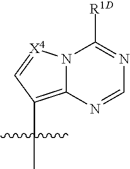

or an optionally substituted

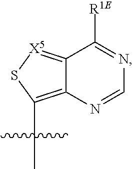

wherein X¹ can be N (nitrogen) or CR^{C1}; X² can be N (nitrogen) or CR^{C2}; X³ can be N (nitrogen) or CR^{C3}; X⁴ can be N (nitrogen) or CR^{C4}; X⁵ can be N (nitrogen) or CR^{C5}; and R^{C1}, R^{C2}, R^{C3}, R^{C4} and R^{C5} can be independently hydrogen or halogen; R^{1B}, R^{1C}, R^{1D} and R^{1E} can be independently hydrogen, halogen, hydroxy, an unsubstituted C₁₋₄ alkyl, an unsubstituted C₂₋₄ alkenyl, an unsubstituted C₃-C₆ cycloalkyl, an unsubstituted C₁₋₄ alkoxy or NR^{A1}R^{A2}; and R^{A1} and R^{A2} can be independently selected from hydrogen, hydroxy, an unsubstituted C₁₋₄ alkyl, an unsubstituted C₁₋₄ alkoxy and —C(═O)R^{C6}, wherein R^{C6} can be hydrogen, an unsubstituted C₁₋₄ alkyl or an unsubstituted C₃₋₄ monocyclic cycloalkyl; R¹ can be hydrogen or an unsubstituted C₁₋₄ alkyl; R^{2A} can be hydrogen or an unsubstituted C₁₋₄ alkyl; R^{2B} can be halogen, OH, —O—C(═O)—C₁₋₄ alkyl or —O—C(═O)—CH(R¹′)—NH₂, wherein R¹′ can be H, —CH₃, —CH(CH₃)₂, —CH₂—CH(CH₃)₂ or —CH(CH₃)—CH(CH₃)₂; R^{3A} can be hydrogen, an unsubstituted or a substituted C₁₋₄ alkyl, an unsubstituted or a substituted C₂₋₄ alkenyl or an unsubstituted or a substituted C₂₋₄ alkynyl, wherein when the C₁₋₄ alkyl, the C₂₋₄ alkenyl and the C₂₋₄ alkynyl are substituted, each can be independently substituted with 1 or more fluoros; R^{3B} can be halogen, OH, —O—C(═O)—C₁₋₄ alkyl or —O—C(═O)—CH(R¹″)—NH₂, wherein R¹″ is H, —CH₃, —CH(CH₃)₂, —CH₂—CH(CH₃)₂ or —CH(CH₃)—CH(CH₃)₂; R^{4A} can be —(CR^{D1}R^{E1})(CR^{D2}R^{E2})n-R^{F1}, —(CR^{G1}R^{H1})—O—R^{J1}, —O—(CR^{K1}R^{L1})—R^{M1} or —(CR^{N1}R^{O1})p-R^{P1}; wherein R^{D1}, R^{E1}, R^{D2} and R^{E2} can be independently selected from hydrogen, halogen, hydroxy and an unsubstituted C₁₋₃ alkyl; n can be 0 or 1; and R^{F1} can be an unsubstituted or a substituted aryl, an unsubstituted or a substituted heteroaryl or an unsubstituted or a substituted heterocyclyl; or R^{D1} and R^{E1} can be taken together with the carbon to which R^{D1} and R^{E1} are attached to form an unsubstituted cyclopropyl ring; and R^{D2} and R^{E2} can be independently selected from hydrogen, halogen, hydroxy and an unsubstituted C₁₋₃ alkyl; n can be 1; and R^{F1} can be an unsubstituted or a substituted aryl, an unsubstituted or a substituted heteroaryl or an unsubstituted or a substituted heterocyclyl; or R^{D1} and R^{E2} can be independently selected from hydrogen, halogen, hydroxy and an unsubstituted C₁₋₃ alkyl; R^{E1} and R^{D2} can be taken together with the carbon to which R^{E1} and R^{D2} are attached to form an unsubstituted cyclopropyl ring; n can be 1; and R^{F1} can be an unsubstituted or a substituted aryl, an unsubstituted or a substituted heteroaryl or an unsubstituted or a substituted heterocyclyl; or R^{D1} and R^{E2} can be independently selected from hydrogen, halogen, hydroxy and an unsubstituted C₁₋₃ alkyl; R^{E1} and R^{D2} together form a double bond; n can be 1; and R^{F1} can be an unsubstituted or a substituted aryl, an unsubstituted or a substituted heteroaryl or an unsubstituted or a substituted heterocyclyl; R^{G1}, R^{H1}, R^{K1}, R^{L1}, R^{N1} and R^{O1} can be independently selected from hydrogen, halogen, hydroxy and an unsubstituted C₁₋₃ alkyl; R^{J1} and R^{M1} can be independently an unsubstituted or a substituted aryl, an unsubstituted or a substituted heteroaryl or an unsubstituted or a substituted heterocyclyl; R^{P1} can be an unsubstituted or a substituted heteroaryl; and p can be 3 or 4; R^{4B} can be halogen, cyano, azido, —C(═O)NH₂, an unsubstituted or a substituted C₁₋₄ alkyl, an unsubstituted or a substituted C₂₋₄ alkenyl, an unsubstituted or a substituted C₂₋₄ alkynyl or an unsubstituted or a substituted C₃-C₄ cycloalkyl, wherein when the C₁₋₄ alkyl is substituted, the C₁₋₄ alkyl can be substituted with 1 or more substituents independently selected from halogen, OH and cyano, and wherein when the C₂₋₄ alkenyl is substituted, the C₂₋₄ alkenyl can be substituted independently with 1 or more halogens; Z¹ can be CR^{5A}R^{5B}, O (oxygen), S (sulfur) or N (an unsubstituted C₁₋₄ alkyl); R^{5A} and R^{5B} can be independently hydrogen, halogen, cyano or an unsubstituted or a substituted C₁₋₄ alkyl, wherein when the C₁₋₄ alkyl is substituted, the C₁₋₄ alkyl can be substituted with 1 or more substituents independently selected from fluoro and hydroxy; or R^{5A} and R^{5B} together with the carbon R^{5A} and R^{5B} are attached can form a double bond optionally substituted with one or two halogen, R^{5A} and R^{5B} together with the carbon R^{5A} and R^{5B} are attached can form an unsubstituted cyclopropyl or R^{5A} and R^{5B} together with the carbon R^{5A} and R^{5B} are attached can form an unsubstituted or a substituted oxetane, wherein when the oxetane is substituted, the oxetane can be substituted independently with 1 or 2 halogens; or R^{2A} and R^{2B} together with the carbon R^{2A} and R^{2B} are attached can form a 3, 4 or 5 membered monocyclic cycloalkyl or a 3, 4 or 5 membered monocyclic heterocyclyl; or R^{3A} and R^{3B} together with the carbon R^{3A} and R^{3B} are attached can form a 3, 4 or 5 membered monocyclic cycloalkyl or a 3, 4 or 5 membered monocyclic heterocyclyl; or R^{4B} and R^{3B} together with the carbon R^{4B} and R^{3B} are attached can form an unsubstituted oxetane; or R^{4B} and R^{5B} together with the carbon R^{4B} and R^{5B} are attached can form an unsubstituted cyclopropyl; or R¹ and R^{5B} together with the carbon R¹ and R^{5B} are attached can form an unsubstituted cyclopropyl; or when Z¹ is O, then R^{2B} and R^{4B} can be connected via —(CH₂)y-O—, wherein y can be 1 or 2,

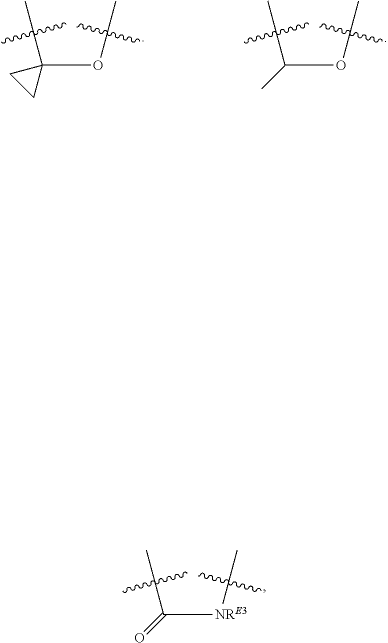

wherein NR^{E3} can be R^{E3} can be hydrogen or an unsubstituted C₁₋₇ alkyl or

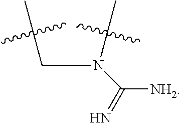 [SMILES: *CN(*)C(=N)N]

The 5-membered ring of Formula (I) can be a carbocyclyl or a heterocyclyl. In some embodiments, the 5-membered ring of Formula (I) can be a carbocyclyl when Z¹ is CR^{5A}R^{5B}. Various substituents can be present at R^{5A} and R^{5B}. In some embodiments, R^{5A} and R^{5B} can be each hydrogen such that Z¹ is CH₂. In some embodiments, at least one of R^{5A} and R^{5B} can be halogen, for example F. In some embodiments, R^{5A} and R^{5B} can be each halogen. When R^{5A} and R^{5B} are each halogen, the halogens can be the same or different. An example of R^{5A} and R^{5B} each being halogen is CF₂. In some embodiments, at least one of R^{5A} and R^{5B} can be cyano. In some embodiments, at least one of R^{5A} and R^{5B} can be an unsubstituted C₁₋₄ alkyl. Examples of unsubstituted C₁₋₄ alkyls include methyl, ethyl, n-propyl, isopropyl, n-butyl, isobutyl and tert-butyl. In some embodiments, one of R^{5A} and R^{5B} can be an unsubstituted C₁₋₄ alkyl (such as those described herein); and the other R^{5A} and R^{5B} can be hydrogen. In some embodiments, at least one of R^{5A} and R^{5B} can be a substituted C₁₋₄ alkyl (such as those C₁₋₄ alkyls described herein) substituted with 1 or more substituents independently selected from fluoro and hydroxy. Those skilled in the art understand that when Z¹ is CR^{5A}R^{5B}, the carbon to which R^{5A} and R^{5B} are attached can be a stereocenter. In some embodiments, the carbon to which R^{5A} and R^{5B} are attached can be in the R-configuration

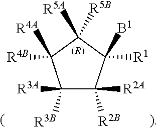

In other embodiments, the carbon to which R^{5A} and R^{5B} are attached can be in the S-configuration

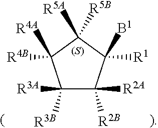

In some embodiments, Z¹ can be CR^{5A}R^{5B}, wherein R^{5A} and R^{5B} together with the carbon R^{5A} and R^{5B} are attached form a double bond optionally substituted with one or two halogen. For example, Z¹ can be C═CH₂, C═CCl₂ or C═CF₂. In other embodiments, when Z¹ is CR^{5A}R^{5B}, R^{5A} and R^{5B} together with the carbon R^{5A} and R^{5B} are attached form an unsubstituted cyclopropyl. In still other embodiments, when Z¹ is CR^{5A}R^{5B}, R^{5A} and R^{5B} together with the carbon R^{5A} and R^{5B} are attached form an unsubstituted or a substituted oxetane, wherein when the oxetane is substituted, the oxetane is substituted independently with 1 or 2 halogens (for example, fluoro or chloro). When R^{5A} and R^{5B} together with the carbon R^{5A} and R^{5B} are attached form an unsubstituted cyclopropyl or an unsubstituted or a substituted oxetane, the 5-membered ring of Formula (I) and the unsubstituted cyclopropyl or an unsubstituted or a substituted oxetane are connected in a spiro-manner.

As described herein, the 5-membered ring of Formula (I) can be a heterocyclyl. In some embodiments, Z¹ can be S (sulfur). In other embodiments, Z¹ can be N (an unsubstituted C₁₋₄ alkyl). Exemplary C₁₋₄ alkyls are described herein, for example, methyl, ethyl, n-propyl, isopropyl, n-butyl, iso-butyl and tert-butyl.

The 2′-position of the 5-membered ring of Formula (I) can have present various substituents. The positions of the 5-membered ring as referred to herein are as follows:

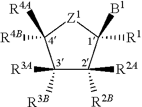

In some embodiments, R^{2A} can be hydrogen. In other embodiments, R^{2A} can be an unsubstituted C₁₋₄ alkyl. Suitable examples of C₁₋₄ alkyls are provided herein and include methyl, ethyl, n-propyl, isopropyl, n-butyl, isobutyl and tert-butyl. In some embodiments, R^{2B} can be OH. In other embodiments, R^{2B} can be —O—C(═O)—C₁₋₄ alkyl, such as —O—C(═O)—CH₃, —O—C(═O)—CH₂CH₃, —O—C(═O)—CH₂CH₂CH₃, —O—C(═O)—CH₂CH₂CH₂CH₃, —O—C(═O)—CH(CH₃)₂ and —O—C(═O)—C(CH₃)₃. In still other embodiments, R^{2B} can be an alpha-amino acid linked via its carboxy group. Alpha-amino acids are known to those skilled in the art, and include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In yet still other embodiments, R^{2B} can be —O—C(═O)—CH(R¹′)—NH₂, wherein R¹′ can be H, —CH₃, —CH(CH₃)₂, —CH₂—CH(CH₃)₂ or —CH(CH₃)—CH(CH₃)₂. In some embodiments, R^{2A} can be halogen. Examples of halogens include F, Cl, Br and I. In yet still other embodiments, R^{2A} and R^{2B} together with the carbon R^{2A} and R^{2B} are attached form a 3, 4 or 5 membered monocyclic cycloalkyl or a 3, 4 or 5 membered monocyclic heterocyclyl. The 3, 4 or 5 membered monocyclic heterocyclyl formed from R^{2A} and R^{2B} being taken together with the carbon to which R^{2A} and R^{2B} are attached include, but are not limited to, oxetane and thietane. As described herein, R^{2B} can be —O—C(═O)—C₁₋₄ alkyl, an alpha-amino acid linked via its carboxy group or —O—C(═O)—CH(R¹″)—NH₂, and those skilled in the art understand that when R^{2B} is one of the aforementioned substituents, that compound of Formula (I) can be considered a prodrug of the corresponding a compound of Formula (I) where R^{2B} is OH. In some embodiments, In other embodiments, R^{2B} can be halogen, —O—C(═O)—C₁₋₄ alkyl or —O—C(═O)—CH(R¹′)—NH₂ and/or R^{3B} can be halogen —O—C(═O)—C₁₋₄ alkyl or —O—C(═O)—CH(R¹″)—NH₂.

A variety of substituents can also be present at the 3′-position of the 5-membered ring of Formula (I). In some embodiments, R^{3A} can be hydrogen. In other embodiments, R^{3A} can be an unsubstituted C₁₋₄ alkyl, such as methyl, ethyl, n-propyl, isopropyl, n-butyl, isobutyl and tert-butyl. In still other embodiments, R^{3A} can be a substituted C₁₋₄ alkyl (such as those described herein) substituted with 1 or more fluoros. In some embodiments, R^{3A} can be an unsubstituted C₂₋₄ alkenyl. In other embodiments, R^{3A} can be a substituted C₂₋₄ alkenyl substituted with 1 or more fluoros. In still other embodiments, R^{3A} can be an unsubstituted C₂₋₄ alkynyl. In yet still other embodiments, R^{3A} can be a substituted C₂₋₄ alkynyl substituted with 1 or more fluoros.

Further groups can be present at the 3′-position of the 5-membered ring of Formula (I). In some embodiments, R^{3B} can be OH. In other embodiments, R^{3B} can be —O—C(═O)—C₁₋₄ alkyl. Exemplary C₁₋₄ alkyls are described herein. In still other embodiments, R^{3B} can be an alpha-amino acid linked via its carboxy group. Several alpha-amino acids are known to those skilled in the art, and described herein. In yet still other embodiments, R^{3B} can be —O—C(═O)—CH(R¹″)—NH₂, wherein R¹″ can be H, —CH₃, —CH(CH₃)₂, —CH₂—CH(CH₃)₂ or —CH(CH₃)—CH(CH₃)₂. In some embodiments, R^{3B} can be halogen. For example, R^{3B} can be fluoro. In some embodiments, R^{3A} and R^{3B} together with the carbon R^{3A} and R^{3B} are attached form a 3, 4 or 5 membered monocyclic cycloalkyl or a 3, 4 or 5 membered monocyclic heterocyclyl. When R^{3B} is —O—C(═O)—C₁₋₄ alkyl, an alpha-amino acid linked via its carboxy group or —O—C(═O)—CH(R¹″)—NH₂, the compound of Formula (I) can be considered a prodrug of the corresponding a compound of Formula (I) where R^{3B} is OH. For example, when R^{2B} and R^{3B} are each —O—C(═O)—C₁₋₄ alkyl, that compound of Formula (I) can be considered a prodrug a compound of Formula (I) where R^{2B} and R^{3B} are each —OH. An example of this type of prodrug is Compound 26, wherein Compound 26 being considered a prodrug of Compound 12. The structure of Compounds 12 and 26 are provided herein.

As with the other positions on the 5-membered ring, the substituents present at the 4′-position can vary. In some embodiments, R^{4A} can be —(CR^{D1}R^{E1})(CR^{D2}R^{E2})n-R^{F1}. Further, the substituents for R^{D1}, R^{E1}, R^{D2} and R^{E2} can also vary. In some embodiments, n can be 0. In other embodiments, n can be 1. In some embodiments, R^{D1}, R^{E1}, R^{D2} and R^{E2} can be each hydrogen, such that —(CR^{D1}R^{E1})(CR^{D2}R^{E2})n-R^{F1} can be —CH₂—R^{F1} or —CH₂CH₂—R^{F1}. In some embodiments, at least one of R^{D1} and R^{E1} can be hydrogen; and the other of R^{D1} and R^{E1} can be a non-hydrogen moiety as described herein. For example, one of R^{D1} and R^{E1} can be hydrogen; and the other of R^{D1} and R^{E1} can be halogen, or one of R^{D1} and R^{E1} can be hydrogen; the other of R^{D1} and R^{E1} can be hydroxy; and one of R^{D1} and R^{E1} can be hydrogen; the other of R^{D1} and R^{E1} can be an unsubstituted C₁₋₃ alkyl. In other embodiments, R^{D1} and R^{E1} can be each halogen, for example, fluoro. In some embodiments, at least one of R^{D2} and R^{E2} can be hydrogen; and the other of R^{D2} and R^{E2} can be a non-hydrogen moiety as described herein. For example, one of R^{D2} and R^{E2} can be hydrogen; and the other of R^{D2} and R^{E2} can be halogen, or one of R^{D2} and R^{E2} can be hydrogen; the other of R^{D2} and R^{E2} can be hydroxy; and one of R^{D2} and R^{E2} can be hydrogen; the other of R^{D2} and R^{E2} can be an unsubstituted C₁₋₃ alkyl. In other embodiments, R^{D2} and R^{E2} can be each halogen, for example, fluoro.

As described herein the substituents, R^{D1}, R^{E1}, R^{D2} and R^{E2} can vary. In some embodiments, R^{4A} can be —(CR^{D1}R^{E1})(CR^{D2}R^{E2})n-R^{F1}, wherein two of R^{D1}, R^{E1}, R^{D2} and R^{E2} can be taken together to form an unsubstituted cyclopropyl or a double bond. Examples of when two of R^{D1}, R^{E1}, R^{D2} and R^{E2} can be taken together to form an unsubstituted cyclopropyl include the embodiments described in this paragraph. In some embodiments, R^{D1} and R^{E1} can be taken together with the carbon to which R^{D1} and R^{E1} are attached to form an unsubstituted cyclopropyl ring; and R^{D2} and R^{E2} can be independently selected from hydrogen, halogen, hydroxy and an unsubstituted C₁₋₃ alkyl; and R^{E1} can be an unsubstituted or a substituted aryl, an unsubstituted or a substituted heteroaryl or an unsubstituted or a substituted heterocyclyl. In other embodiments, R^{D1} and R^{E2} can be independently selected from hydrogen, halogen, hydroxy and an unsubstituted C₁₋₃ alkyl; R^{E1} and R^{D2} can be taken together with the carbon to which R^{E1} and R^{D2} are attached to form an unsubstituted cyclopropyl ring; and R^{F1} can be an unsubstituted or a substituted aryl, an unsubstituted or a substituted heteroaryl or an unsubstituted or a substituted heterocyclyl.

In some embodiments, R^{D1} and R^{E2} can be independently selected from hydrogen, halogen, hydroxy and an unsubstituted C₁₋₃ alkyl; R^{E1} and R^{D2} together form a double bond; and R^{F1} can be an unsubstituted or a substituted aryl, an unsubstituted or a substituted heteroaryl or an unsubstituted or a substituted heterocyclyl. Examples of R^{4A} include, but are not limited to, —CH₂—R^{F1}, —CH₂CH₂—R^{F1}, —CF₂—R^{F1}, —CH(OH)—R^{F1},

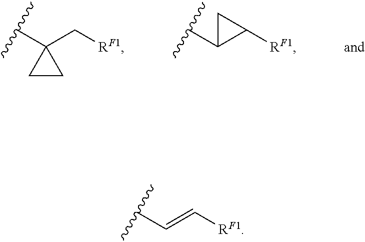

As described herein, R^{F1} can be various ring structures. In some embodiments, R^{F1} can be an unsubstituted aryl. In other embodiments, R^{F1} can be a substituted aryl. When the aryl is monocyclic, R^{F1} can be an unsubstituted or a substituted phenyl. Multicyclic aryl groups can also be present at R^{F1}, such as naphthyl and anthracenyl. In some embodiments, R^{F1} can be an unsubstituted heteroaryl. In other embodiments, R^{F1} can be a substituted heteroaryl. The heteroaryl for R^{F1} can be also monocyclic (such as a 5- or 6-membered monocyclic) or multicyclic (for example, bicyclic). In some embodiments, R^{F1} can be 9- or 10-membered bicyclic heteroaryl. Examples of suitable heteroaryls for R^{F1} include quinolinyl and imidazo[1,2-a]pyridinyl. In still other embodiments, R^{F1} can be an unsubstituted heterocyclyl. In yet still other embodiments, R^{F1} can be a substituted heterocyclyl. The heterocyclyls for R^{F1} can be monocyclic or multicyclic. For example, R^{F1} can be a bicyclic heterocyclyl, such as a 9- or 10-membered bicyclic heterocyclyl. Exemplary further R^{F1} groups include quinazoline, quinazolin-4-one, quinoxaline, isoquinoline, cinnoline, naphthyridine, benzimidazole and benzothiazole.

In other embodiments, R^{4A} can be —(CR^{G1}R^{H1})—O—R^{J1}. As described herein, R^{G1} and R^{H1} can be independently hydrogen, halogen or hydroxy. In some embodiments, R^{G1} and R^{H1} can be each hydrogen, such that R^{4A} can be —CH₂—O—R^{J1}. In some embodiments, at least one of R^{G1} and R^{H1} can be halogen, such as fluoro; and the other of R^{G1} and R^{H1} can be hydrogen. In other embodiments, R^{G1} and R^{H1} can be each halogen. As example of when R^{G1} and R^{H1} are each halogen is —CF₂—O—R^{J1}. In some embodiments, at least one of R^{G1} and R^{H1} can be hydroxy. In some embodiments, at least one of R^{G1} and R^{H1} can be hydrogen. When at least one of R^{G1} and R^{H1} can be hydrogen, —(CR^{G1}R^{H1})—O—R^{J1} can be —CH(CH₃)—O—R^{J1}.

As with R^{F1}, R^{J1} can be various cyclic moieties. In some embodiments, R^{J1} can be an unsubstituted aryl, such as an unsubstituted phenyl or an unsubstituted naphthyl. In other embodiments, R^{J1} can be a substituted aryl, for example, a substituted phenyl or a substituted naphthyl. In some embodiments, R^{J1} is an unsubstituted heteroaryl. In other embodiments, R^{J1} is a substituted heteroaryl. In still other embodiments, R^{J1} is an unsubstituted heterocyclyl. In yet still other embodiments, R^{J1} is a substituted heterocyclyl. The heteroaryl and heterocyclyl for R^{J1} can be monocyclic or bicyclic, for example, R^{J1} can be a 5-membered monocyclic heteroaryl, 6-membered monocyclic heteroaryl, 9-membered bicyclic heteroaryl, 10-membered bicyclic heteroaryl, 5-membered monocyclic heterocyclyl, 6-membered monocyclic heterocyclyl, 9-membered bicyclic heterocyclyl or 10-membered bicyclic heterocyclyl. Examples of cyclic moieties that can be R^{J1} include, but are not limited to, quinolinyl, imidazo[1,2-a]pyridinyl, quinazoline, quinazolin-4-one, quinoxaline, isoquinoline, cinnoline, naphthyridine, benzimidazole and benzothiazole.

In still other embodiments, R^{4A} can be —O—(CR^{K1}R^{L1})—R^{M1}. In some embodiments, R^{K1} and R^{L1} can be each hydrogen, such that R^{4A} can be —O—CH₂—R^{M1}. In some embodiments, at least one of R^{K1} and R^{L1} can be halogen, such as fluoro; and the other of R^{K1} and R^{L1} can be hydrogen. In other embodiments, R^{K1} and R^{H1} can be each halogen, for example, —O—CF₂—R^{M1}. In some embodiments, at least one of R^{K1} and R^{L1} can be hydroxy. In some embodiments, at least one of R^{K1} and R^{J1} can be hydrogen. When at least one of R^{K1} and R^{L1} can be hydrogen, —O—(CR^{K1}R^{L1})—R^{M1} can be —O—CH(CH₃)—R^{M1}.

In some embodiments, R^{M1} can be an unsubstituted aryl, such as an unsubstituted phenyl or an unsubstituted naphthyl. In other embodiments, R^{M1} can be a substituted aryl, for example, a substituted phenyl or a substituted naphthyl. In some embodiments, R^{M1} is an unsubstituted heteroaryl. In other embodiments, R^{M1} is a substituted heteroaryl. The heteroaryl can be a monocyclic heteroaryl (such as a 5- or 6-membered monocyclic heteroaryl) or a bicyclic heteroaryl (such as a 9- or 10-membered bicyclic heteroaryl). In still other embodiments, R^{M1} is an unsubstituted heterocyclyl. In yet still other embodiments, R^{M1} is a substituted heterocyclyl. As with the heteroaryl, the heterocyclyl can be a monocyclic heterocyclyl (such as a 5- or 6-membered monocyclic heterocyclyl) or a bicyclic heterocyclyl (such as a 9- or 10-membered bicyclic heterocyclyl). Examples of R^{M1} group include, but are not limited to, quinolinyl, imidazo[1,2-a]pyridinyl, quinazoline, quinazolin-4-one, quinoxaline, isoquinoline, cinnoline, naphthyridine, benzimidazole and benzothiazole.

In some embodiments, R^{4A} can be —(CR^{N1}R^{O1})p-R^{P1}. As described herein, in some embodiments, p can be 3. In other embodiments, p can be 4. In some embodiments, each R^{N1} and each R^{O1} can be hydrogen. In some embodiments, at least one R^{N1} and/or at least one R^{O1} can be halogen, such as fluoro; and the remaining R^{N1}'s and R^{O1}'s can be hydrogen. In other embodiments, at least one R^{N1} and/or at least one R^{O1} can be hydroxy; and the remaining R^{N1}'s and R^{O1}'s can be hydrogen. In still other embodiments, at least one R^{N1} and/or at least one R^{O1} can be an unsubstituted C₁₋₃ alkyl; and the remaining R^{N1}'S and R^{O1}'s can be hydrogen. As provided herein, R^{P1} can be an unsubstituted or a substituted heteroaryl. In some embodiments, R^{P1} can be an unsubstituted heteroaryl. In other embodiments, R^{P1} can be an unsubstituted heteroaryl. The heteroaryl for R^{P1} can be a monocyclic of a bicyclic heteroaryl. In some embodiments, R^{P1} can be an unsubstituted monocyclic heteroaryl, such as a nitrogen-containing an unsubstituted monocyclic heteroaryl. In other embodiments, R^{P1} can be a substituted monocyclic heteroaryl, for example, a nitrogen-containing a substituted monocyclic heteroaryl.

When substituted, R^{F1}, R^{J1}, R^{M1} and R^{P1} can be substituted 1, 2, 3 or more than 3 times with a variety of groups. When more than one group is present, one or more of the groups can be the same. The groups on R^{F1}, R^{J1}, R^{M1} and R^{P1}, when substituted, can be different from each other. Examples of groups that can be present on a substituted R^{F1}, R^{J1} and/or R^{M1} include, but are not limited to, halogen (for example, F, Cl and Br), cyano, an unsubstituted C₁₋₄ alkyl, an unsubstituted C₁₋₄ haloalkyl (such as CH₂F, CHF₂, CF₃, CH₂Cl, CHCl₂ and C₁₋₃), an unsubstituted monocyclic C₃₋₆ cycloalkyl, an optionally substituted C-carboxy, an optionally substituted N-amido, amino, a mono-substituted amine, a di-substituted amine, —NH—C(═O)-unsubstituted C₁₋₈ alkyl, —NH—C(═O)—O-unsubstituted C₁₋₈ alkyl, —NH—C(═O)-unsubstituted C₃₋₆ cycloalkyl and —NH—C(═O)—O-unsubstituted C₃₋₆ cycloalkyl. Further examples that can be present on a substituted R^{F1}, R^{J1}, R^{M1} and/or R^{P1} include, but are not limited to, an unsubstituted C₁₋₄ alkoxy, an unsubstituted or a substituted phenyl and an unsubstituted or a substituted monocyclic heteroaryl (such as an unsubstituted or a substituted 5- or 6-membered heteroaryl). Prodrugs of compounds of Formula (I) can be obtained by substituting R^{F1}, R^{J1}, R^{M1} and/or R^{P1} with an appropriate group. As an example, when R^{F1}, R^{J1}, R^{M1} and/or R^{P1} is substituted with —NH—C(═O)-unsubstituted C₁₋₈ alkyl, —NH—C(═O)—O-unsubstituted C₁₋₈ alkyl, —NH—C(═O)-unsubstituted C₃₋₆ cycloalkyl and —NH—C(═O)—O-unsubstituted C₃₋₆ cycloalkyl, that compound of Formula (I) can be considered a prodrug of a compound of Formula (I) where an NH₂ group replaces the —NH—C(═O)-unsubstituted C₁₋₈ alkyl, —NH—C(═O)—O-unsubstituted C₁₋₈ alkyl, —NH—C(═O)-unsubstituted C₃₋₆ cycloalkyl or —NH—C(═O)—O-unsubstituted C₃₋₆ cycloalkyl group. A specific example is Compound 20 as described herein can be considered a prodrug of Compound 12. The specific structure of each of Compound 12 and Compound 20 are provided herein.

Exemplary R^{F1}, R^{J1} and R^{M1} groups include, but are not limited to, the following:

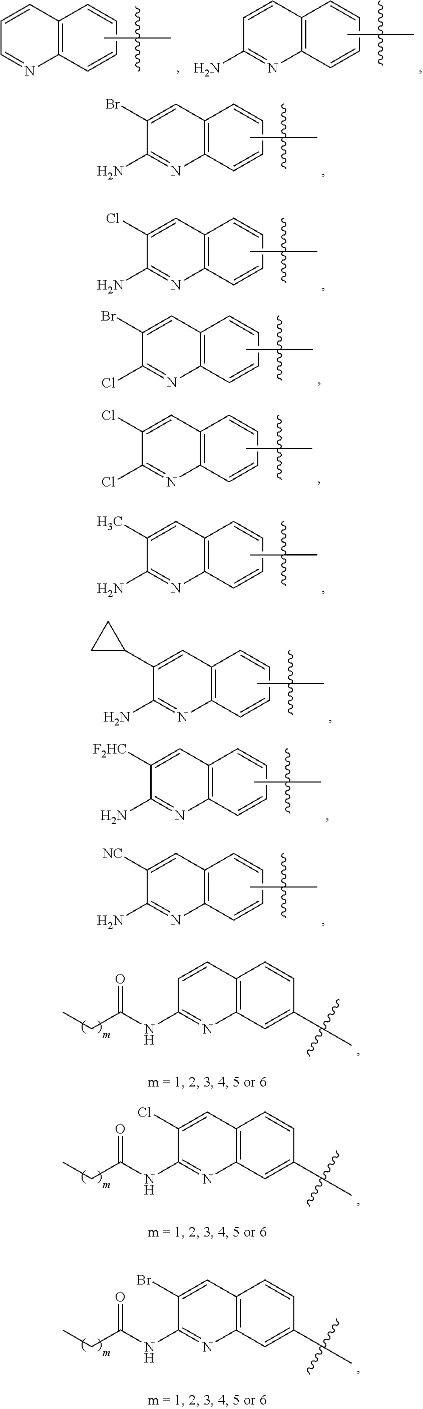

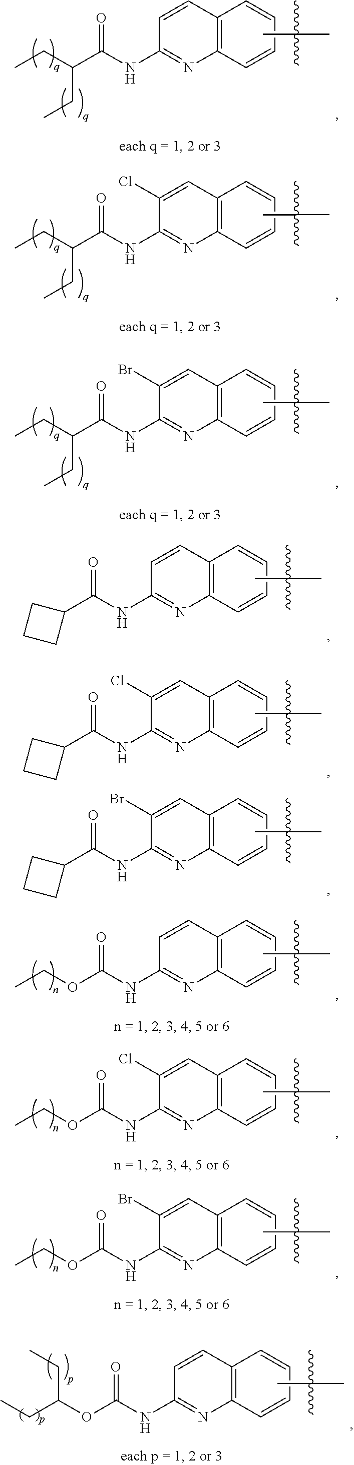

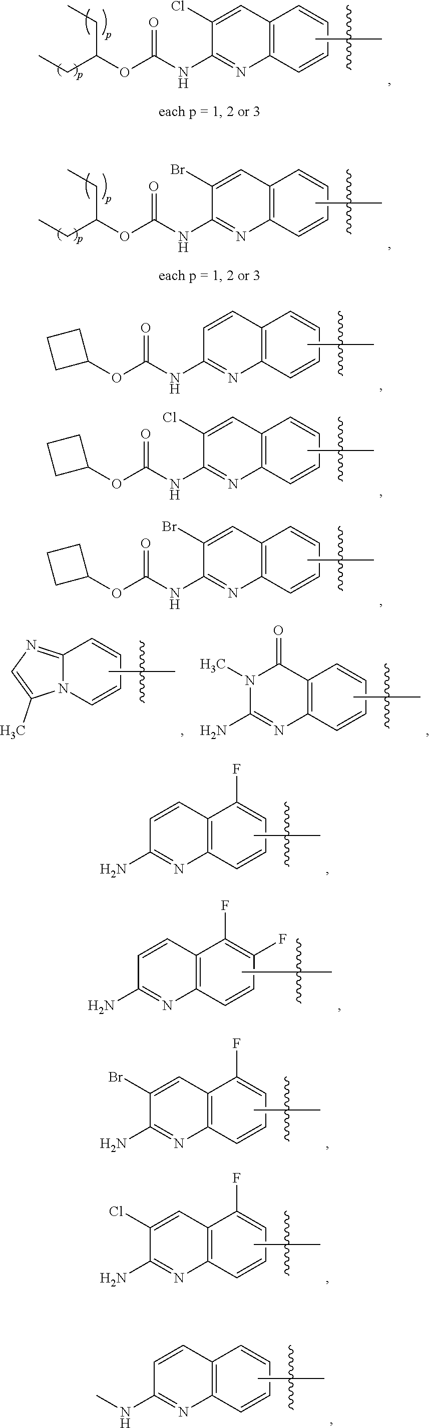

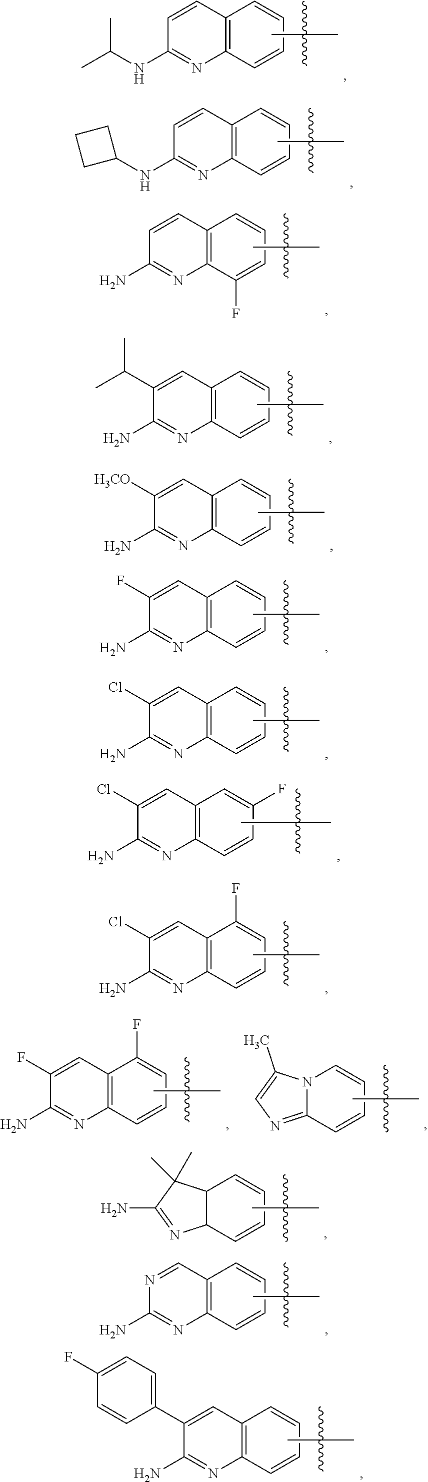

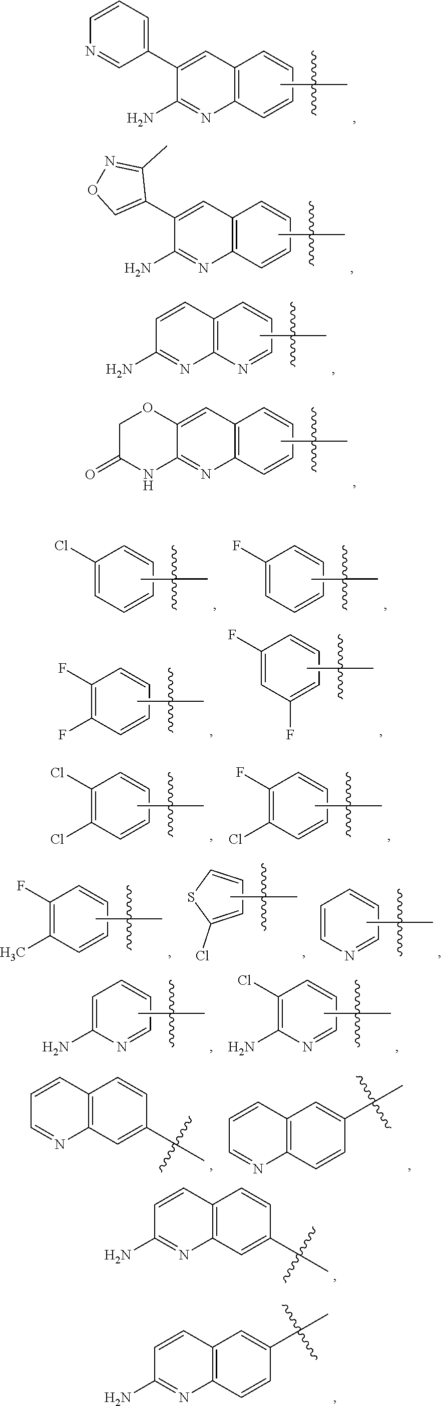

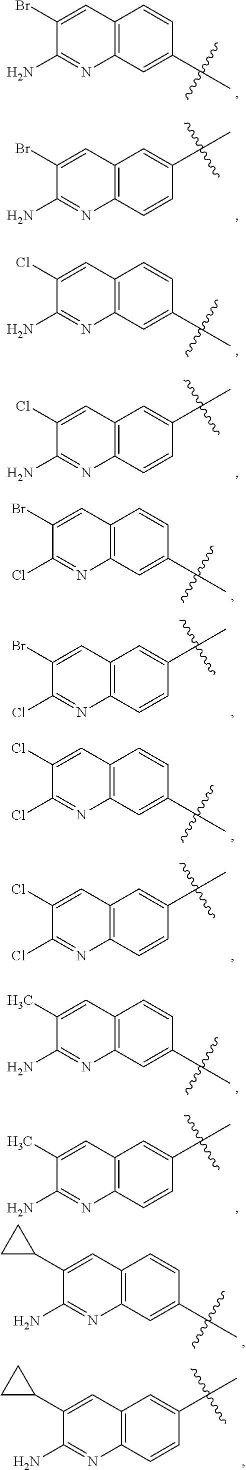

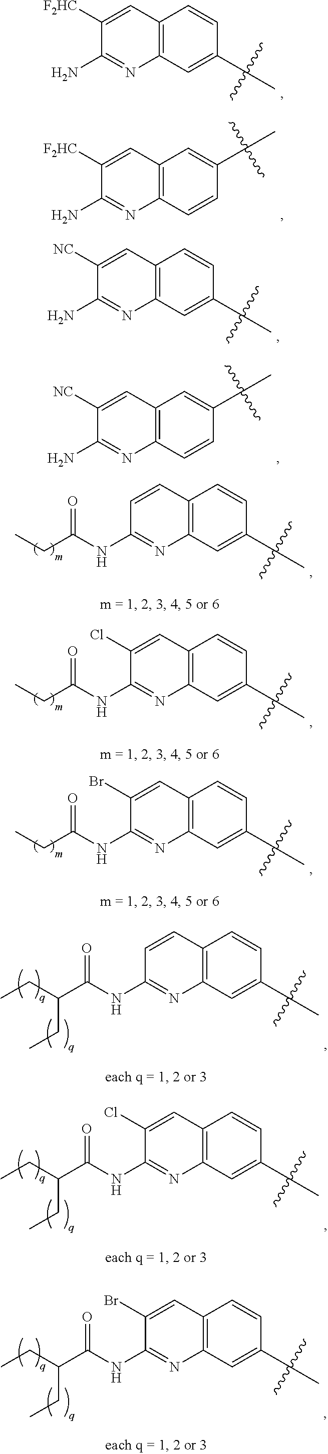

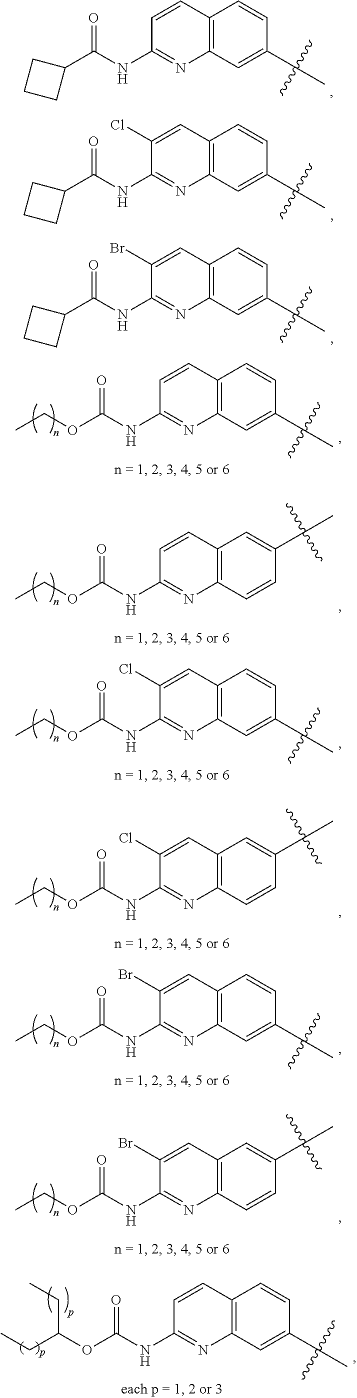

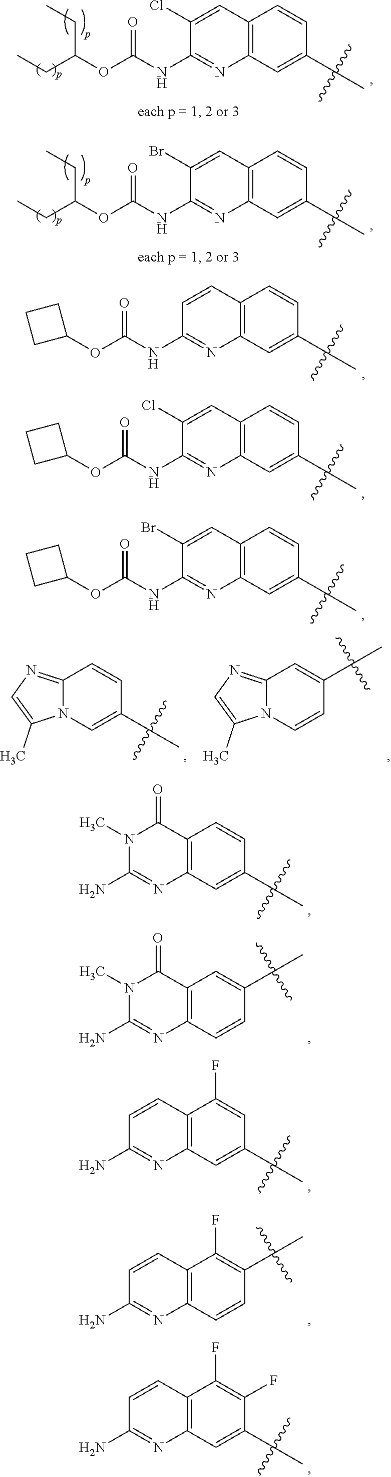

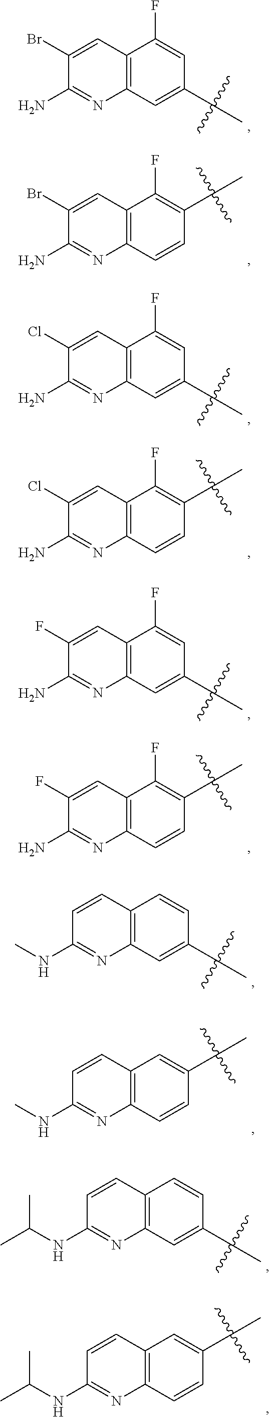

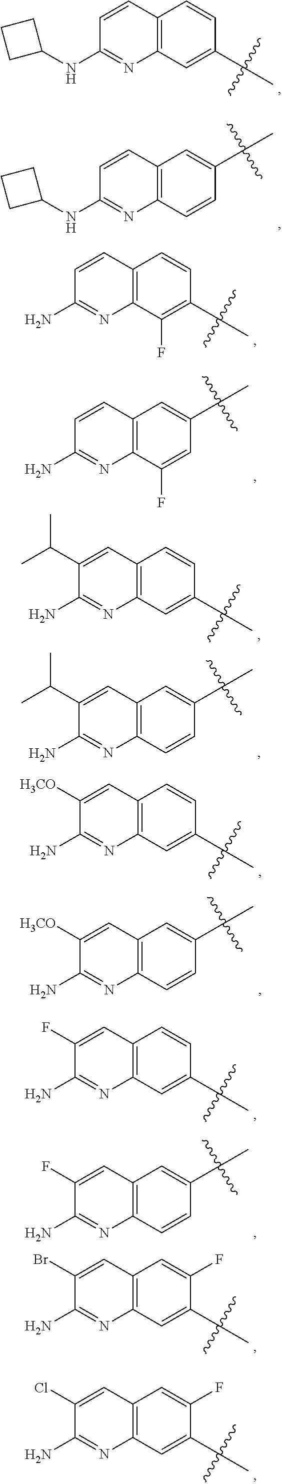

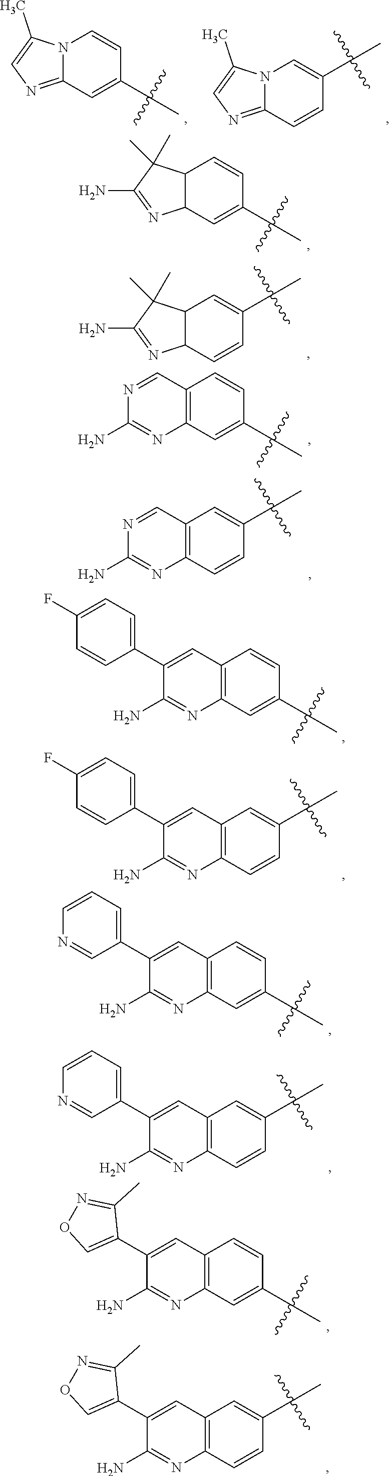

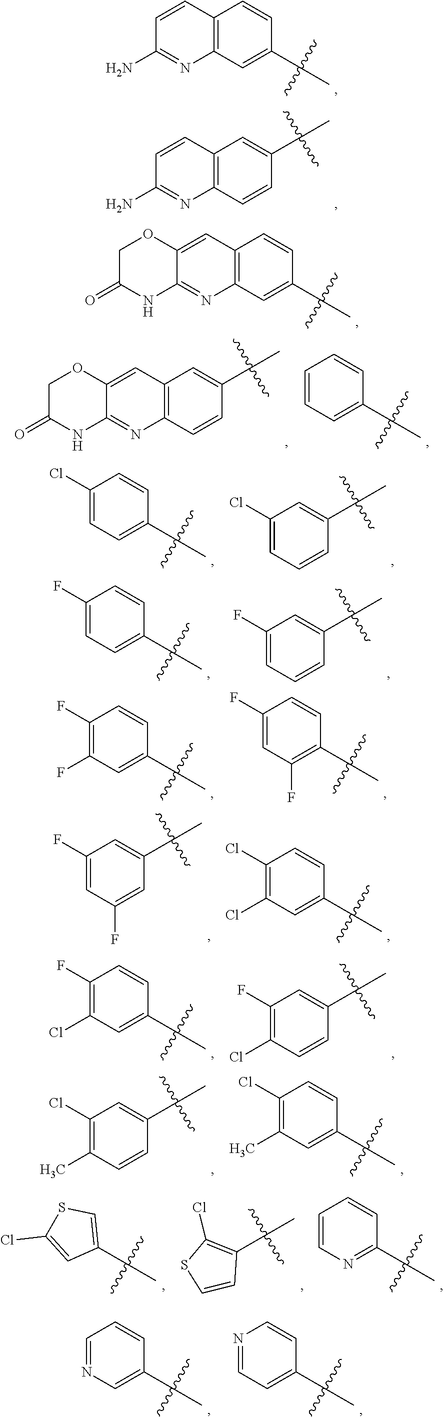

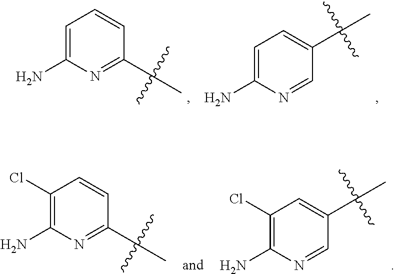 [SMILES: *c1cccc(N)n1 ; *c1ccc(N)nc1 ; *c1ccc(Cl)c(N)n1 ; *c1cnc(N)c(Cl)c1]

A non-limiting list of R^{P1} groups include the following:

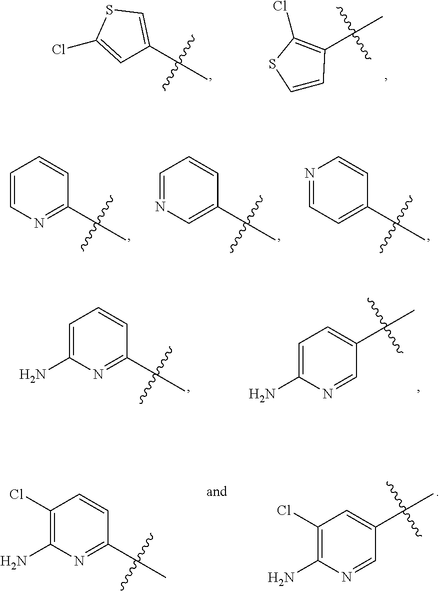

Various other groups can be present at the 4′-position of the 5-membered ring of Formula (I). In some embodiments, R^{4B} can be hydrogen. In other embodiments, R^{4B} can be halogen, such as F. In still other embodiments, R^{4B} can be cyano. In yet still other embodiments, R^{4B} can be azido. In some embodiments, R^{4B} can be —C(═O)NH₂. In other embodiments, R^{4B} can be an unsubstituted C₁₋₄ alkyl, such as methyl, ethyl, n-propyl, isopropyl, n-butyl, isobutyl and tert-butyl. In still other embodiments, R^{4B} can be a substituted C₁₋₄ alkyl substituted with 1 or more substituents independently selected from the halogen (such as F and/or Cl), OH, OCH₃ and cyano. Examples of substituted C₁₋₄ alkyl for R^{4B} include —CH₂F, —CHF₂, —CF₃, —CH₂Cl, —CHCl₂, —CCl₃, —CH₂OH and —CH₂CN. In yet still other embodiments, R^{4B} can be an unsubstituted C₂₋₄ alkenyl. In some embodiments, R^{4B} can be a substituted C₂₋₄ alkenyl substituted independently with 1 or more halogens, for example, fluoro and/or chloro. In other embodiments, R^{4B} can be an unsubstituted C₂₋₄ alkynyl. In still other embodiments, R^{4B} can be a substituted C₂₋₄ alkynyl. Examples of C₂₋₄ alkenyl and C₂₋₄ alkynyl include, but are not limited to, ethenyl, propenyl (straight-chained and branched), butenyl (straight-chained and branched), ethynyl, propynyl (straight-chained and branched) and butynyl (straight-chained and branched). In some embodiments, R^{4B} can be an unsubstituted C₃-C₄ cycloalkyl. In other embodiments, R^{4B} can be a substituted C₃-C₄ cycloalkyl. For example, R^{4B} can be an unsubstituted or a substituted cyclopropyl or an unsubstituted or a substituted cyclobutyl. Alternatively, the 4′-position can be substituted by taking R^{4B} and R^{3B} together with the carbon R^{4B} and R^{3B} are attached form an unsubstituted oxetane. In some embodiments, R^{4B} can be halogen, cyano, azido, —C(═O)NH₂, a substituted C₁₋₄ alkyl substituted with OH, OCH₃ or cyano, an unsubstituted or a substituted C₃₋₄ alkenyl, an unsubstituted or a substituted C₂₋₄ alkynyl or an unsubstituted or a substituted C₃-C₄ cycloalkyl.

As provided herein, the 2′-position and the 4′-position can be connected via various moieties. In some embodiments, the 2′-position and the 4′-position can be connected via a —(CH₂)y-O— moiety, wherein y can be 1 or 2. In some embodiments, R^{2B} and R^{4B} can be connected via —(CH₂)—O—. In other embodiments, R^{2B} and R^{4B} are connected via —CH₂CH₂—O—. In some embodiments, the 2′-position and the 4′-position can be connected via

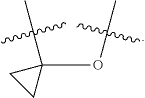 [SMILES: *OC1(*)CC1]

In other embodiments, the 2′-position and the 4′-position can be connected via

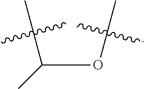 [SMILES: *OC(*)C]

In still other embodiments, the 2′-position and the 4′-position can be connected via

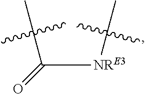

wherein R^{E3} can be hydrogen or an unsubstituted C₁₋₇ alkyl, for example,

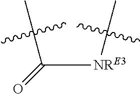

can be

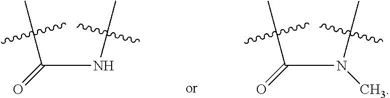 [SMILES: *NC(*)=O ; "or" ; *C(=O)N(*)C]

In yet still other embodiments, the 2′-position and the 4′-position can be connected via

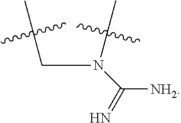 [SMILES: *CN(*)C(=N)N]

In any embodiments of this paragraph, Z¹ can be O (oxygen).

The base, B¹, can be an optionally substituted, N-linked, 9-membered heteroaryl, such as those described herein. In some embodiments, B¹ can be an optionally substituted

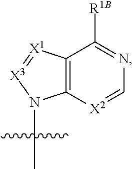

such as an optionally substituted

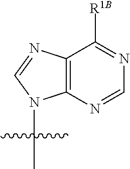

and an optionally substituted

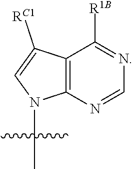

In some embodiments, X¹ can be N (nitrogen). In other embodiments, X¹ can be CR^{C1}. In some embodiments, X² can be N (nitrogen). In other embodiments, X² can be CR^{C2}. In some embodiments, X³ can be N (nitrogen). In other embodiments, X³ can be CR^{C3}. In some embodiments, X⁴ can be N (nitrogen). In other embodiments, X⁴ can be CR^{C4}. In some embodiments, R^{C1}, R^{C2}, R^{C3} and/or R^{C4} can be hydrogen. In some embodiments, R^{C1}, R^{C2}, R^{C3} and/or R^{C4} can be halogen. In some embodiments, R^{C2}, R^{C3} and/or R^{C4} can be an unsubstituted C₁₋₄ alkyl. In other embodiments, B¹ can be

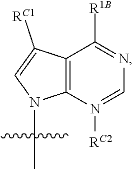

for example, B¹ can be

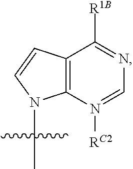

wherein R^{C2} can be halogen (such as F, Cl or Br). In still other embodiments, B¹ can be an optionally substituted

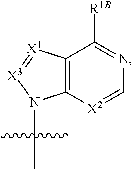

such as

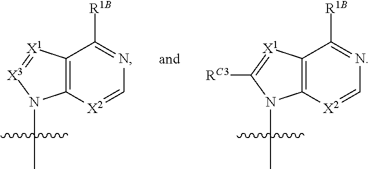

In some embodiments, R^{1B} can be hydrogen, such that B¹ can be an optionally substituted

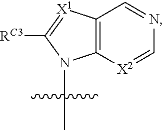

an optionally substituted

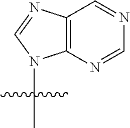 [SMILES: *n1cnc2cncnc21]

and an optionally substituted

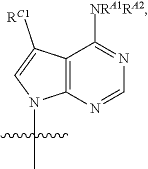

In other embodiments, R^{1B} can be hydroxy or an unsubstituted C₁₋₄ alkoxy. In still other embodiments, R^{1B} can be an unsubstituted C₁₋₄ alkyl, for example an unsubstituted C₁₋₄ alkyl described herein, or an unsubstituted C₂₋₄ alkenyl. In yet still other embodiments, R^{1B} can be an unsubstituted C₃-C₆ cycloalkyl. In some embodiments, R^{1B} can be NR^{A1}R^{A2}, such that B¹ can be an optionally substituted

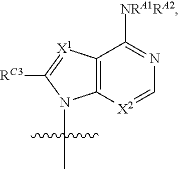

an optionally substituted

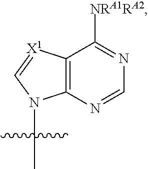

an optionally substituted

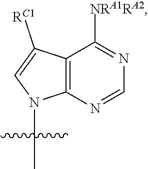

an optionally substituted

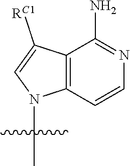

and an optionally substituted

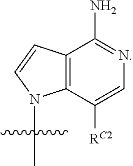

In some embodiments of this paragraph, R^{C1} can be an unsubstituted C₁₋₄ alkyl. In other embodiments of this paragraph, R^{C1} can be hydrogen. In still other embodiments of this paragraph, R^{C1} can be halogen, for example, F, Cl or Br. In some embodiments, B¹ can be an unsubstituted

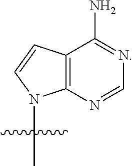 [SMILES: *n1ccc2c(N)ncnc21]

In other embodiments, B¹ can be a substituted

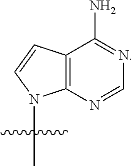 [SMILES: *n1ccc2c(N)ncnc21]

For example,

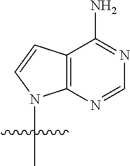 [SMILES: *n1ccc2c(N)ncnc21]

can be substituted with one or more substituents selected from halogen and an unsubstituted C₁₋₄ alkyl.

In other embodiments, B¹ can be an optionally substituted

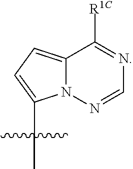

In still other embodiments, B¹ can be an optionally substituted

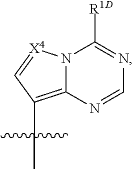

such as an optionally substituted

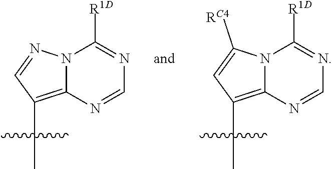

In some embodiments, R^{C4} can be hydrogen. In other embodiments, R^{C4} can be halogen. In still other embodiments, R^{C4} can be an unsubstituted C₁₋₄ alkyl. In yet still other embodiments, B¹ can be an optionally substituted

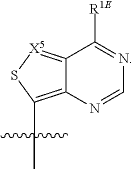

In some embodiments, when B¹ is

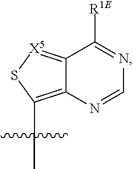

then X⁵ can be N (nitrogen). In other embodiments, when B¹ is

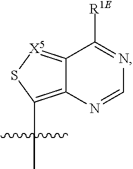

then X⁵ can be CR^{C5}. In some embodiments, R^{C5} can be hydrogen. In other embodiments, R^{C5} can be halogen. In still other embodiments, R^{C5} can be an unsubstituted C₁₋₄ alkyl.

In some embodiments, B¹ can be an unsubstituted or a substituted

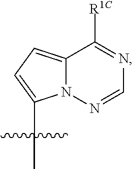

an unsubstituted or a substituted

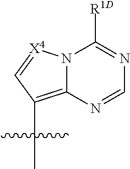

or an unsubstituted or a substituted

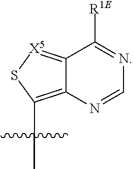

In other embodiments, B¹ can be an unsubstituted or a substituted

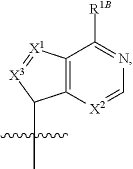

wherein X² can be CR^{C2}. In still other embodiments, B¹ can be an unsubstituted or a substituted

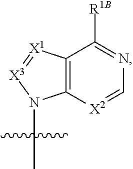

wherein X³ can be N. In yet still other embodiments, B¹ can be an unsubstituted or a substituted

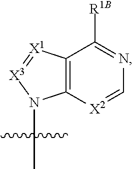

wherein X¹ can be CR^{C1}; and R^{C1} can be hydroxy, an unsubstituted C₂₋₄ alkenyl, an unsubstituted C₁₋₄ alkoxy or NR^{A1}R^{A2}.

As described herein, R^{1C}, R^{1D} and/or R^{1E} can be hydrogen, hydroxy, an unsubstituted C₁₋₄ alkyl or NR^{A1}R^{A2}. In some embodiments, R^{1C} can be hydrogen. In other embodiments, R^{1C} can be hydroxy. In still other embodiments, R^{1C} can be an unsubstituted C₁₋₄ alkyl. In yet still other embodiments, R^{1C} can be an unsubstituted C₂₋₄ alkenyl. In some embodiments, R^{1C} can be an unsubstituted C₁₋₄ alkoxy. In other embodiments, R^{1C} can be an unsubstituted C₃-C₆ cycloalkyl. In still other embodiments, R^{1C} can be NR^{A1}R^{A2}. In some embodiments, R^{1D} can be hydrogen. In other embodiments, R^{1D} can be hydroxy. In still other embodiments, R^{1D} can be an unsubstituted C₁₋₄ alkyl. In yet still other embodiments, R^{1D} can be an unsubstituted C₂₋₄ alkenyl. In some embodiments, R^{1D} can be an unsubstituted C₁₋₄ alkoxy. In other embodiments, R^{1D} can be an unsubstituted C₃-C₆ cycloalkyl. In still other embodiments, R^{1D} can be NR^{A1}R^{A2}. In some embodiments, R^{1E} can be hydrogen. In other embodiments, R^{1E} can be hydroxy. In still other embodiments, R^{1E} can be an unsubstituted C₁₋₄ alkyl. In yet still other embodiments, R^{1E} can be an unsubstituted C₂₋₄ alkenyl. In some embodiments, R^{1E} can be an unsubstituted C₁₋₄ alkoxy. In other embodiments, R^{1E} can be an unsubstituted C₃-C₆ cycloalkyl. In still other embodiments, R^{1E} can be NR^{A1}R^{A2}.

When R^{1B}, R^{1C}, R^{1D} and/or R^{1E} are NR^{A1}R^{A2}, R^{A1} and R^{A2} can be independently selected from hydrogen, hydroxy, an unsubstituted C₁₋₄ alkyl, an unsubstituted C₁₋₄ alkoxy and—C(═O)R^{C6}, wherein R^{C6} can be hydrogen, an unsubstituted C₁₋₄ alkyl or an unsubstituted C₃₋₄ monocyclic cycloalkyl. In some embodiments, when R^{1B}, R^{1C}, R^{1D} and/or R^{1E} is NR^{A1}R^{A2}, R^{A1} and R^{A2} can be each hydrogen. For example, B¹ can be an optionally substituted

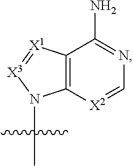

an optionally substituted

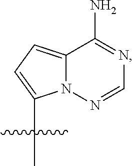 [SMILES: *c1ccc2c(N)ncnn12]

an optionally substituted

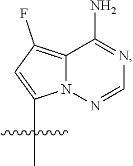 [SMILES: *c1cc(F)c2c(N)ncnn12]

an optionally substituted

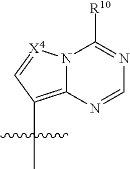

or an optionally substituted

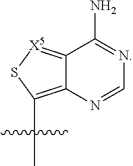

In other embodiments, when R^{1B}, R^{1C}, R^{1D} and/or R^{1E} is NR^{A1}R^{A2}, one of R^{A1} and R^{A2} can be hydrogen, and the other of R^{A1} and R^{A2} can be hydroxy. In still other embodiments, when R^{1B}, R^{1C}, R^{1D} and/or R^{1E} is NR^{A1}R^{A2}, one of R^{A1} and R^{A2} can be hydrogen, and the other of R^{A1} and R^{A2} can be an unsubstituted C₁₋₄ alkyl (for example, methyl, ethyl, n-propyl, isopropyl, n-butyl, isobutyl or tert-butyl). In yet still other embodiments, when R^{1B}, R^{1C}, R^{1D} and/or R^{1E} is NR^{A1}R^{A2}, one of R^{A1} and R^{A2} can be hydrogen, and the other of R^{A1} and R^{A2} can be an unsubstituted C₁₋₄ alkoxy. In some embodiments, when R^{1B}, R^{1C}, R^{1D} and/or R^{1E} is NR^{A1}R^{A2}, one of R^{A1} and R^{A2} can be hydrogen, and the other of R^{A1} and R^{A2} can be —C(═O)R^{C6}, wherein R^{C6} can be hydrogen, an unsubstituted C₁₋₄ alkyl or an unsubstituted C₃₋₄ monocyclic cycloalkyl. In some embodiments, the B¹ groups described herein can be unsubstituted. In some embodiments, the B¹ groups described herein can be substituted, for example, substituted one or more times with a variable selected from halogen and an unsubstituted C₁₋₄ alkyl.

Provided herein are a variety of B groups, including the following:

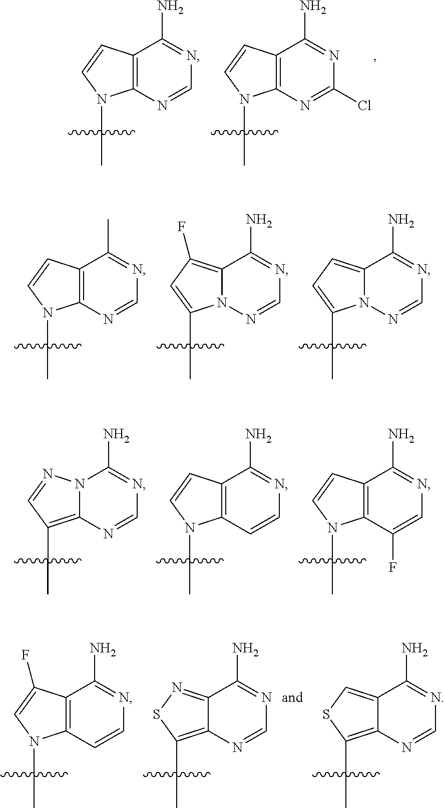

Prodrugs of compounds of Formula (I) can be obtained by substituting B¹ with an appropriate group. For example, when R^{1B}, R^{1C}, R^{1D} and/or R^{1E} is —NH—C(═O)R^{C6}, a compound of Formula (I) with the aforementioned group at R^{1B}, R^{1C}, R^{1D} and/or R^{1E} can be a considered a prodrug of a compound of Formula (I) where R^{1B}, R^{1C}, R^{1D} and/or R^{1E} is NH₂.

The 1′-position of the 5-membered ring of Formula (I) can be unsubstituted or substituted. In some embodiments, R¹ can be hydrogen. In other embodiments, R¹ can be an unsubstituted C₁₋₄ alkyl, such as those described herein.

As provided herein, compounds of Formula (I), or a pharmaceutically acceptable salt thereof, can have various substituents attached to the 5-membered ring of Formula (I). For example, in some embodiments, R¹, R^{2A} and R^{3A} can be each hydrogen; R^{2B} and R^{3B} can be each OH; Z¹ can be CH₂; R^{4B} can be an unsubstituted C₁₋₄ alkyl; B¹ can be a substituted or an unsubstituted

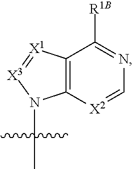

wherein X¹ can be N or CR^{C1}; X² can be N or CR^{C2}; X³ can be N or CR^{C3}; R^{C1}, R^{C2} and R^{C3} can be independently hydrogen, halogen or an unsubstituted C₁₋₄ alkyl; and R^{1B} can be hydrogen or NH₂; and R^{4A}—(CR^{D1}R^{E1})(CR^{D2}R^{E2})n-R^{F1}, wherein R^{D1}, R^{E1}, R^{D2} and R^{E2} can be independently selected from hydrogen, halogen, hydroxy and an unsubstituted C₁₋₃ alkyl; n can be 1; and R^{F1} can be an unsubstituted or a substituted heteroaryl. In other embodiments, R¹, R^{2A} and R^{3A} can be each hydrogen; R^{2B} and R^{3B} can be each OH; Z¹ can be CH₂; R^{4B} can be an unsubstituted C₁₋₄ alkyl; B¹ can be a substituted or an unsubstituted

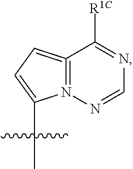

a substituted or an unsubstituted

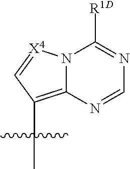

or a substituted or an unsubstituted

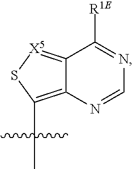

wherein X⁴ can be N or CR^{C4}; X⁵ can be N or CR^{C5}; R^{C4} and R^{C5} can be independently hydrogen, halogen or an unsubstituted C₁₋₄ alkyl; and R^{1C}, R^{1D} and R^{1E} can be independently hydrogen or NH₂; and R^{4A}—(CR^{D1}R^{E1})(CR^{D2}R^{E2})n-R^{F1}, wherein R^{D1}, R^{E1}, R^{D2} and R^{E2} can be independently selected from hydrogen, halogen, hydroxy and an unsubstituted C₁₋₃ alkyl; n can be 1; and R^{F1} can be an unsubstituted or a substituted heteroaryl.

In some embodiments, a compound of Formula (I), or a pharmaceutically acceptable salt thereof, can be any one of the following formulae:

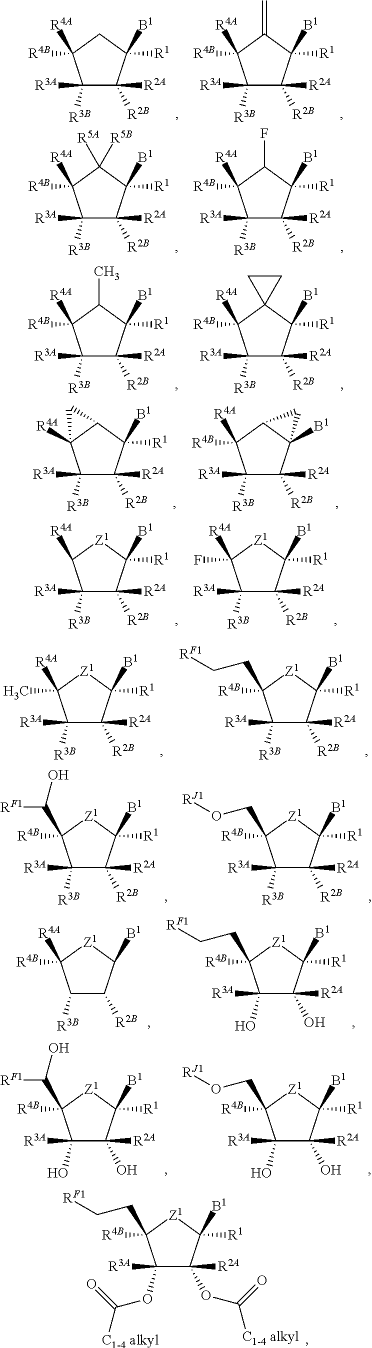

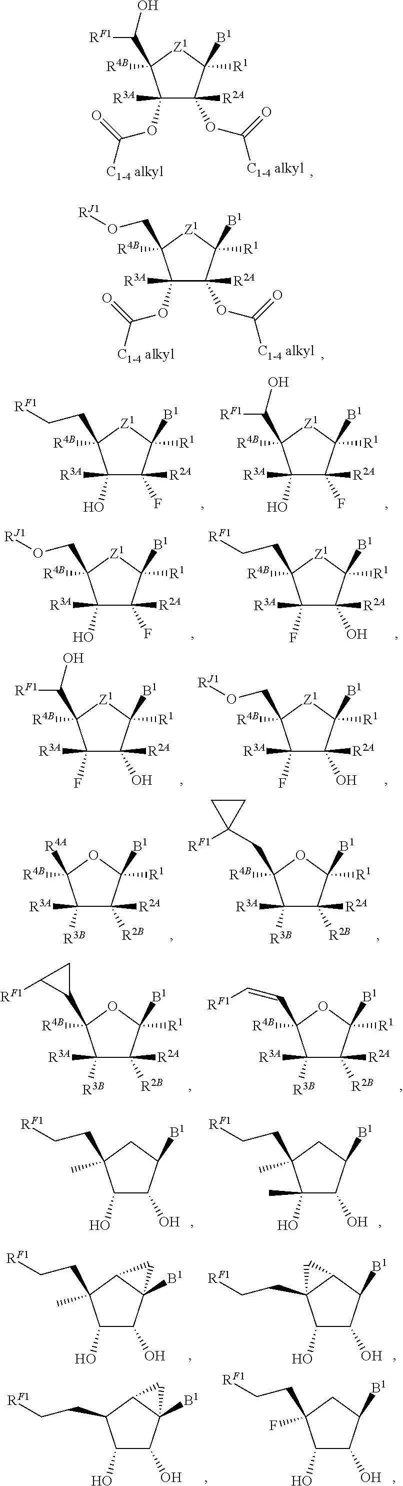

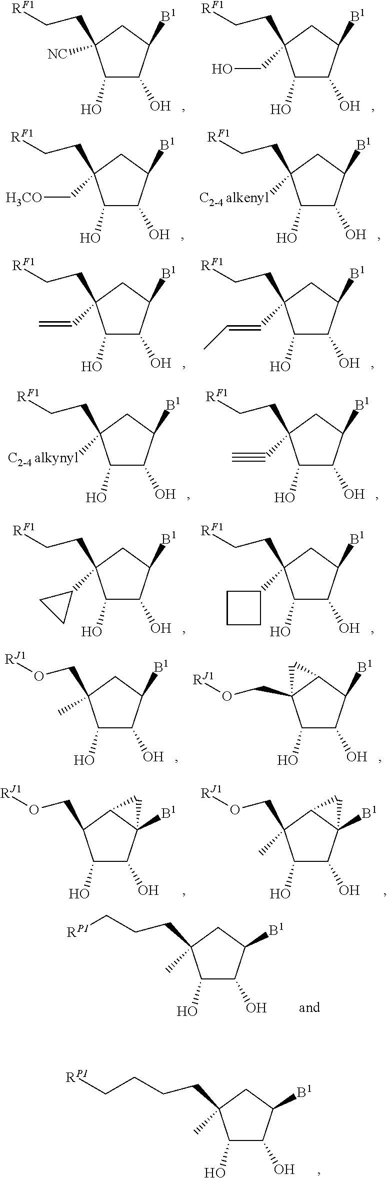

or a pharmaceutically acceptable salt of any of the foregoing. In some embodiments of this paragraph, R^{4B} can be halogen, such as F. In other embodiments of this paragraph, R^{4B} can bean unsubstituted C₁₋₄ alkyl, such as those described herein and including methyl. In still other embodiments of this paragraph, R^{4B} can be hydrogen. In some embodiments of this paragraph, R^{4B} and R^{5B} can be together with the carbon R^{4B} and R^{5B} are attached form an unsubstituted cyclopropyl. In some embodiments of this paragraph, B¹ can be

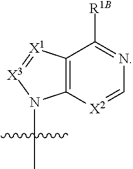

In other embodiments of this paragraph, B¹ can be

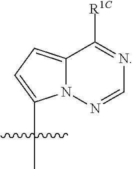

In still other embodiments of this paragraph, B¹ can be

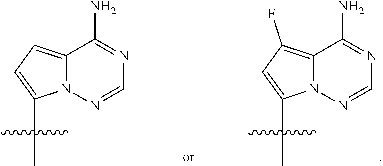 [SMILES: *c1ccc2c(N)ncnn12 ; *c1cc(F)c2c(N)ncnn12]

In yet still other embodiments of this paragraph, B¹ can be

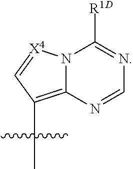

In some embodiments or this paragraph, B¹ can be

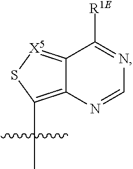

such as

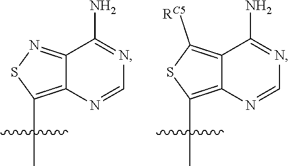

wherein R^{C5} can be halogen or an unsubstituted C₁₋₄ alkyl, or

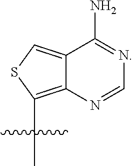 [SMILES: *c1scc2c(N)ncnc12]

In some embodiments of this paragraph, B^{1B} can be

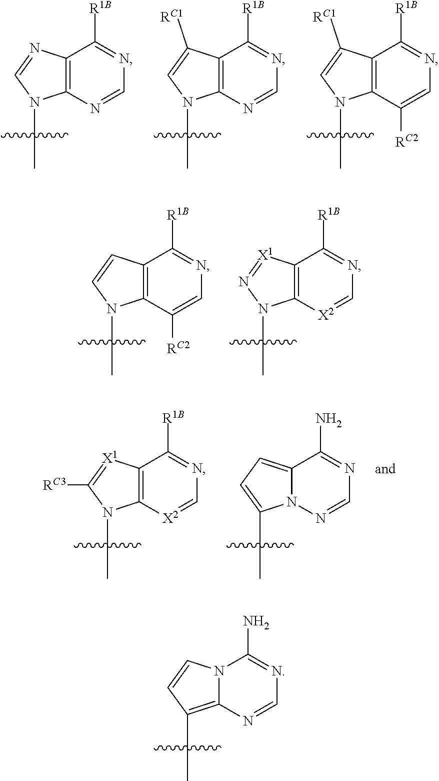

In some embodiments of this paragraph, B¹ can be an unsubstituted

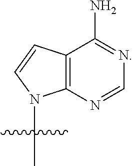 [SMILES: *n1ccc2c(N)ncnc21]

In other embodiments of this paragraph, B¹ can be a substituted

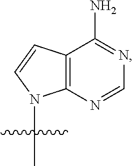 [SMILES: *n1ccc2c(N)ncnc21]

such as those described herein. In some embodiments of this paragraph, B¹ can be an unsubstituted

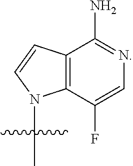 [SMILES: *n1ccc2c(N)ncc(F)c21]

In some embodiments of this paragraph, R^{4A} can be —(CR^{D1}R^{E1})(CR^{D2}R^{E2})ₙ—R^{F1}, for example, —CH₂—R^{F1}, —CF₂—R^{F1} and —CH(OH)—R^{F1}. In some embodiments of this paragraph, R^{4A} can be —(CR^{G1}R^{H1})—O—R^{J1}, such as —CH₂—O—R^{J1}. In some embodiments of this paragraph, R^{4A} can be —O—(CR^{K1}R^{L1})—R^{M1}′ such as —O—CH₂—R^{M1}. In some embodiments of this paragraph, R^{4A} can be —(CR^{N1}R^{O1})p-R^{P1}. In some embodiments of this paragraph, R¹ can be hydrogen. In some embodiments of this paragraph, R^{2A} can be hydrogen. In some embodiments of this paragraph, R^{3A} can be hydrogen. In other embodiments of this paragraph, R^{3A} can be an unsubstituted C₁₋₄ alkyl. In some embodiments of this paragraph, R^{F1}, R^{J1} and/or R^{M1} can be an unsubstituted or a substituted heteroaryl. In some embodiments of this paragraph, R^{F1}, R^{J1} and/or R^{M1} can be a substituted heteroaryl. In some embodiments of this paragraph, R^{F1}, R^{J1} and/or R^{M1} can be an unsubstituted or a substituted heterocyclyl. In some embodiments of this paragraph, R^{F1}, R^{J1} and/or R^{M1} can be a substituted heterocyclyl. In some embodiments of this paragraph, R^{F1}, R^{J1} and/or R^{M1} can be selected from

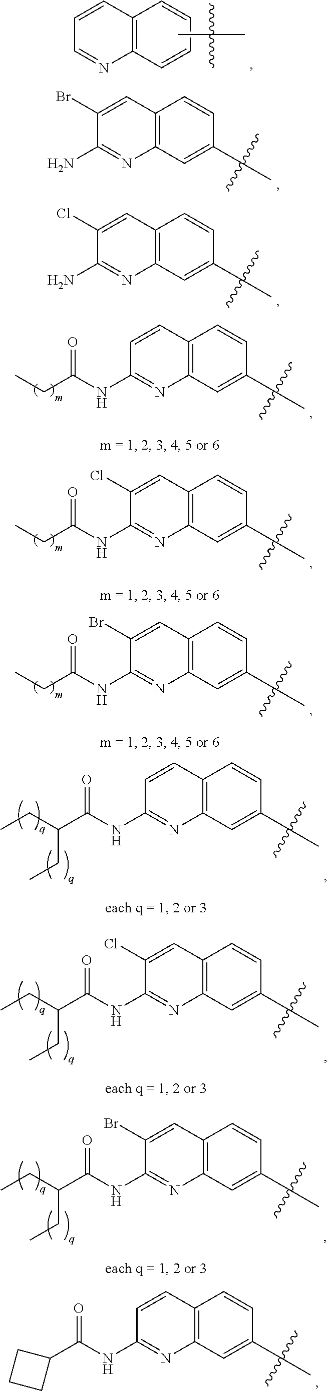

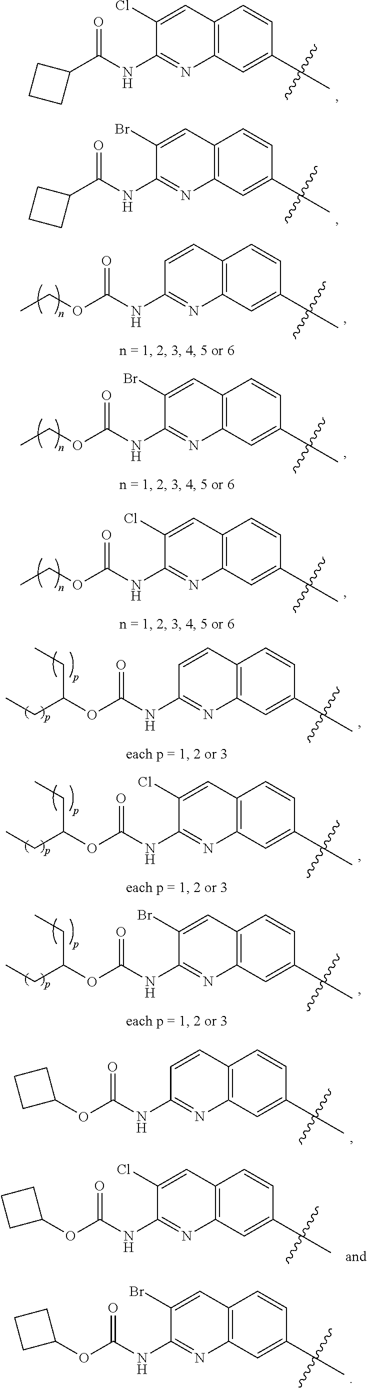

In some embodiments, a compound of Formula (I) can have one of the following structures:

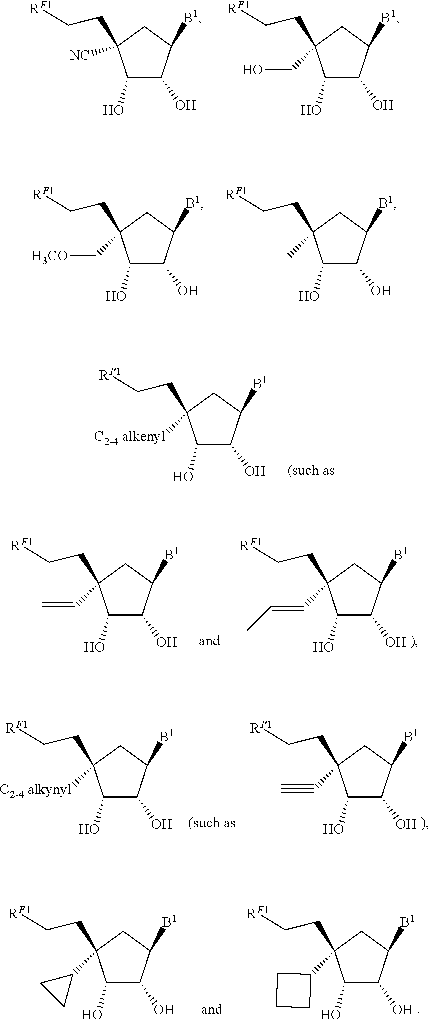

In some embodiments of this paragraph, B¹ can be

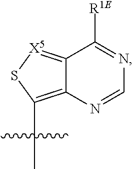

such as

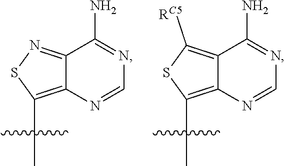

wherein R^{C5} can be halogen or an unsubstituted C₁₋₄ alkyl, or

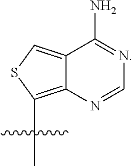 [SMILES: *c1scc2c(N)ncnc12]

In some embodiments of this paragraph, B¹ can be

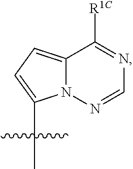

for example,

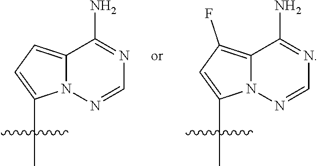 [SMILES: *c1ccc2c(N)ncnn12 ; *c1cc(F)c2c(N)ncnn12]

In some embodiments of this paragraph, B¹ can be

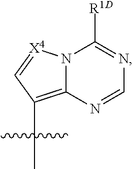

including

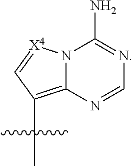

In some embodiments of this paragraph, B¹ can be

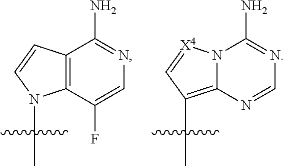

In some embodiments of this paragraph, B¹ can be an unsubstituted or a substituted

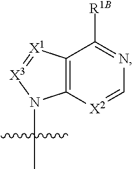

wherein X² can be CR^{C2}; an unsubstituted or a substituted

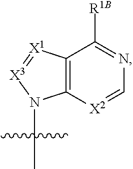

wherein X³ can be N; or an unsubstituted or a substituted

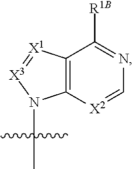

wherein X¹ can be CR^{C1}, and R^{C1} can be hydroxy, an unsubstituted C₂₋₄ alkenyl, an unsubstituted C₁₋₄ alkoxy or NR^{A1}R^{A2}.

Examples of compounds of Formula (I), or a pharmaceutically acceptable salt thereof, include the following:

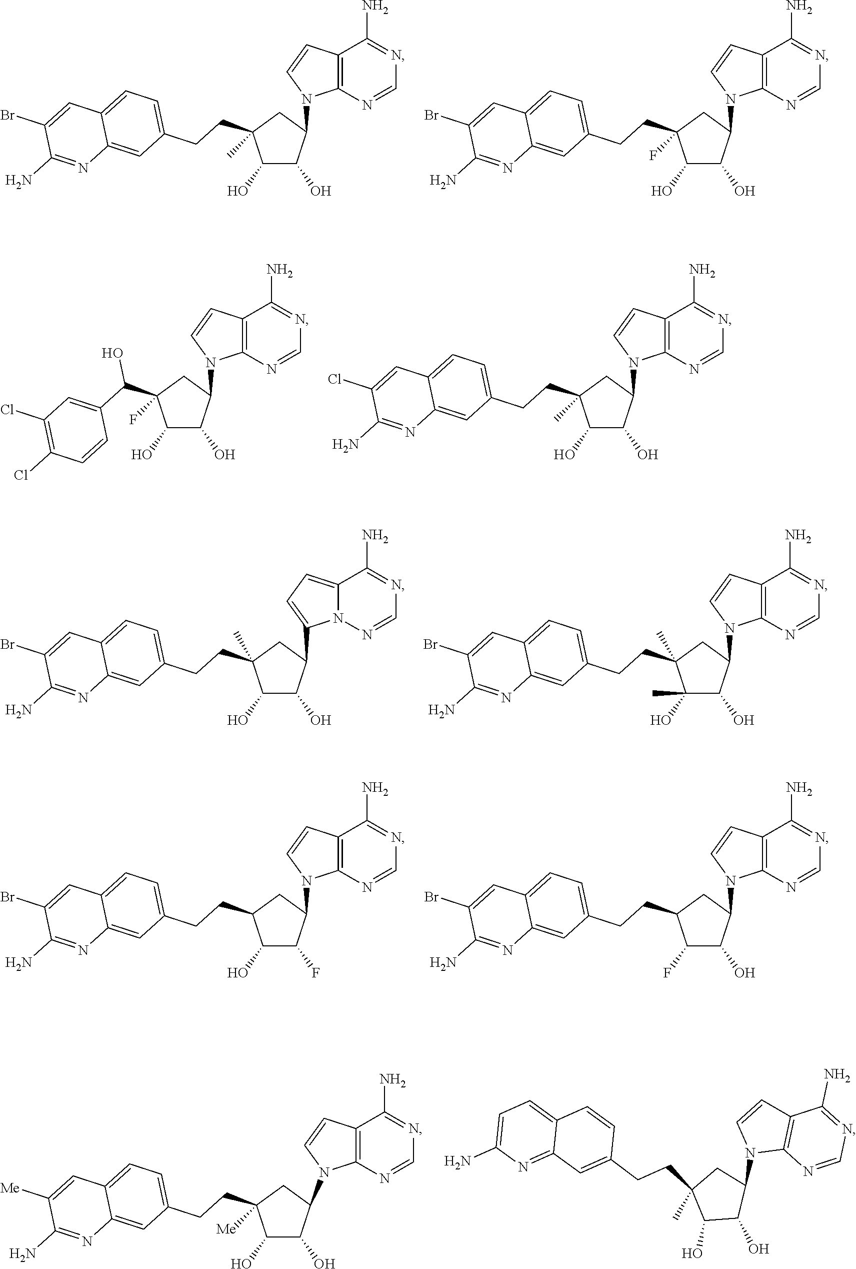

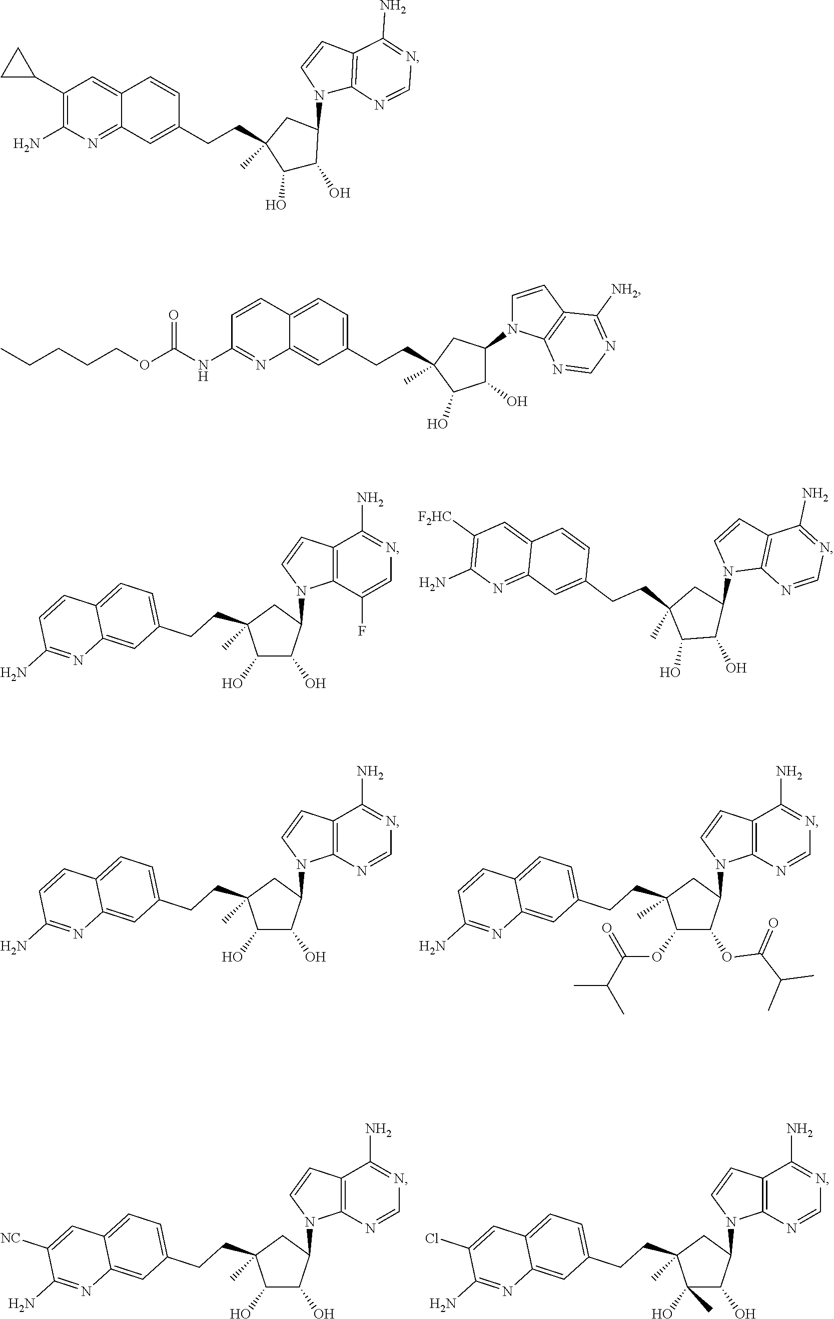

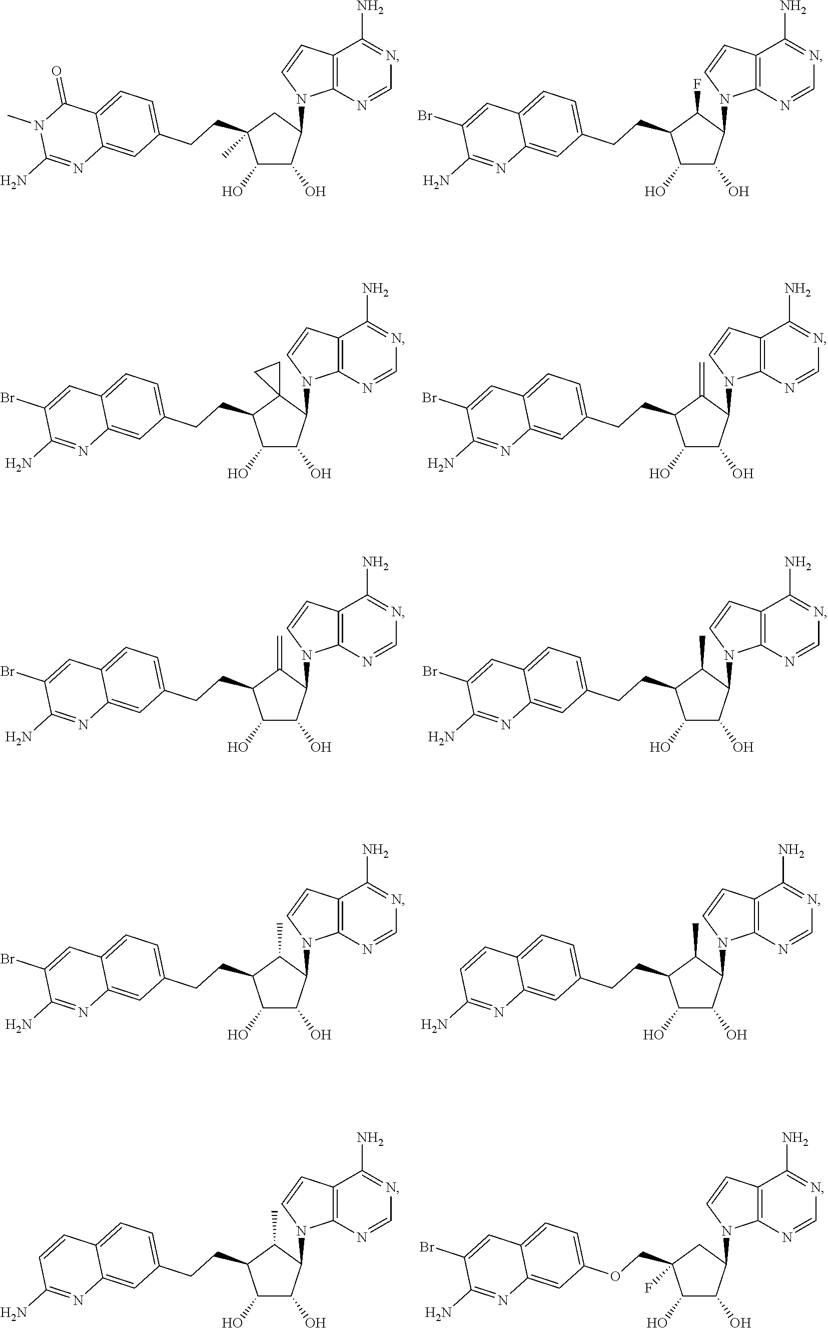

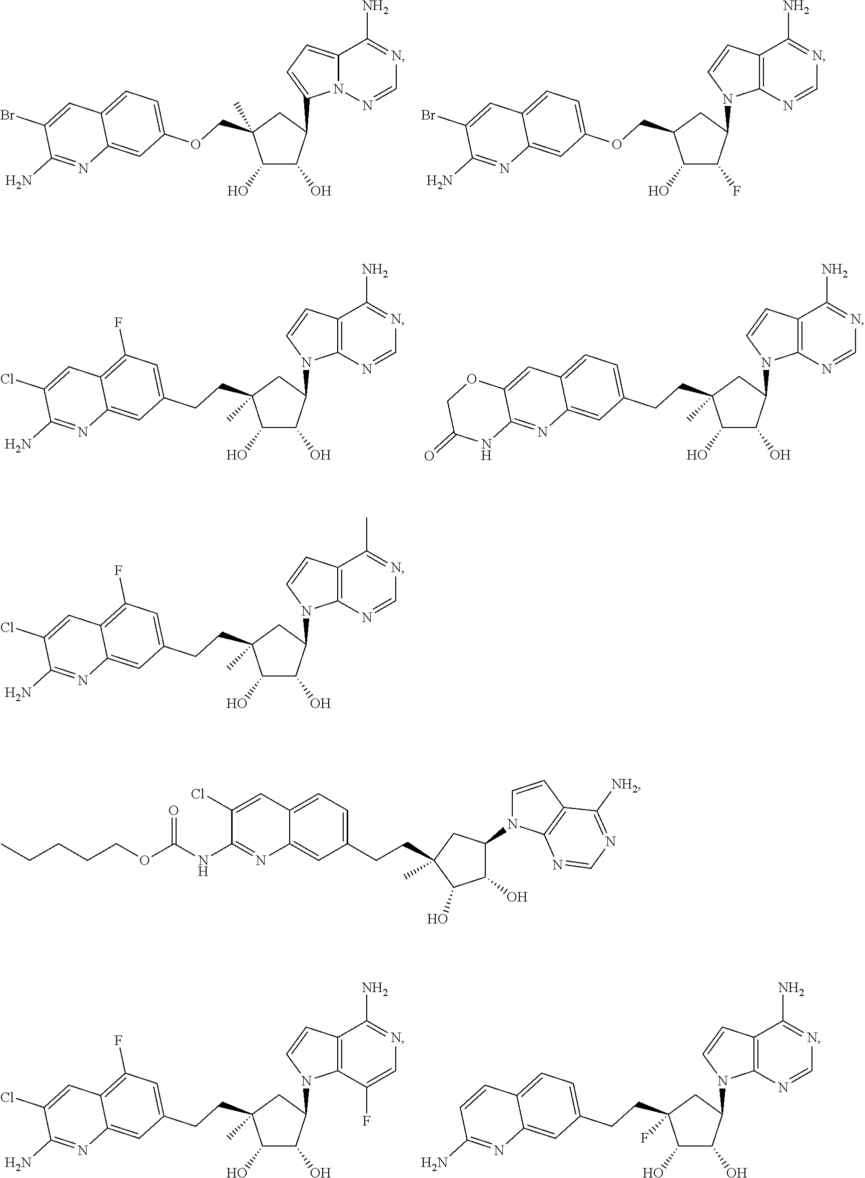

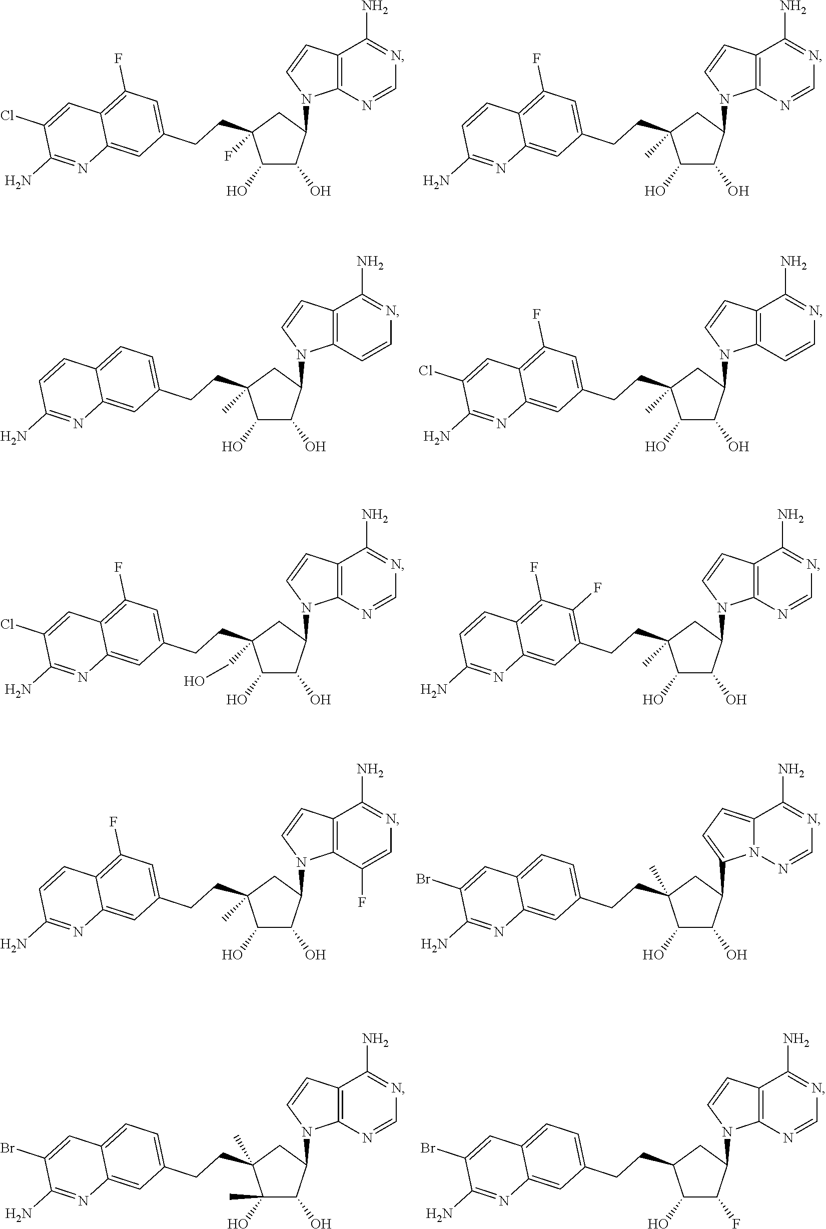

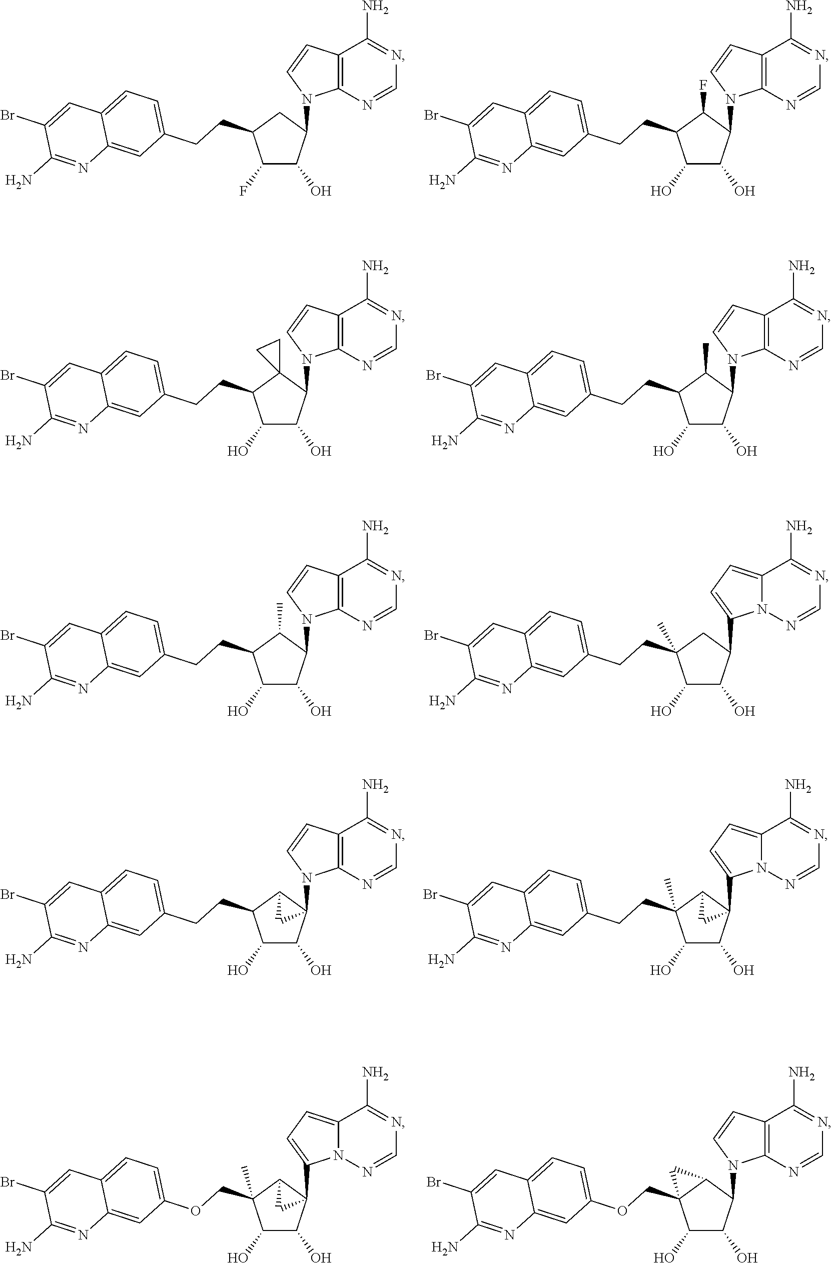

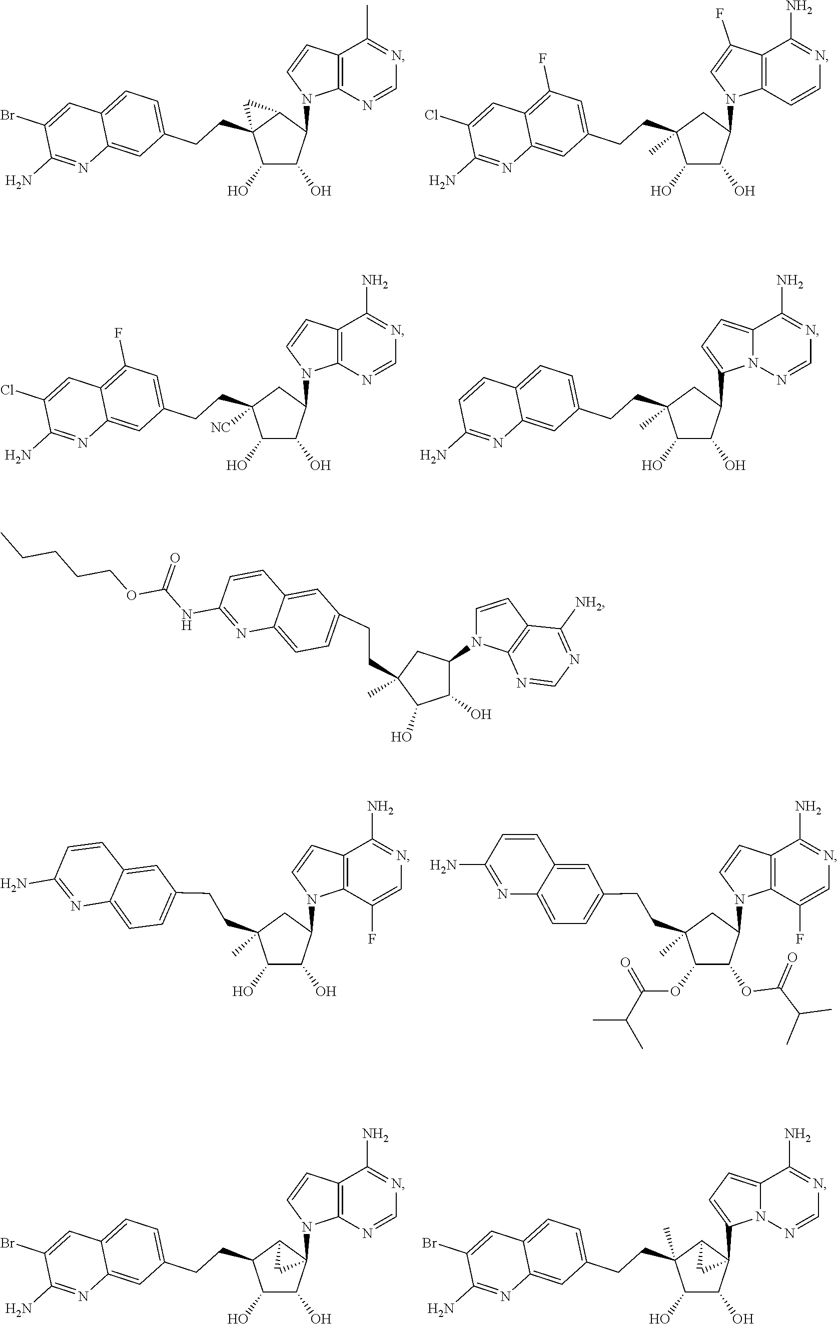

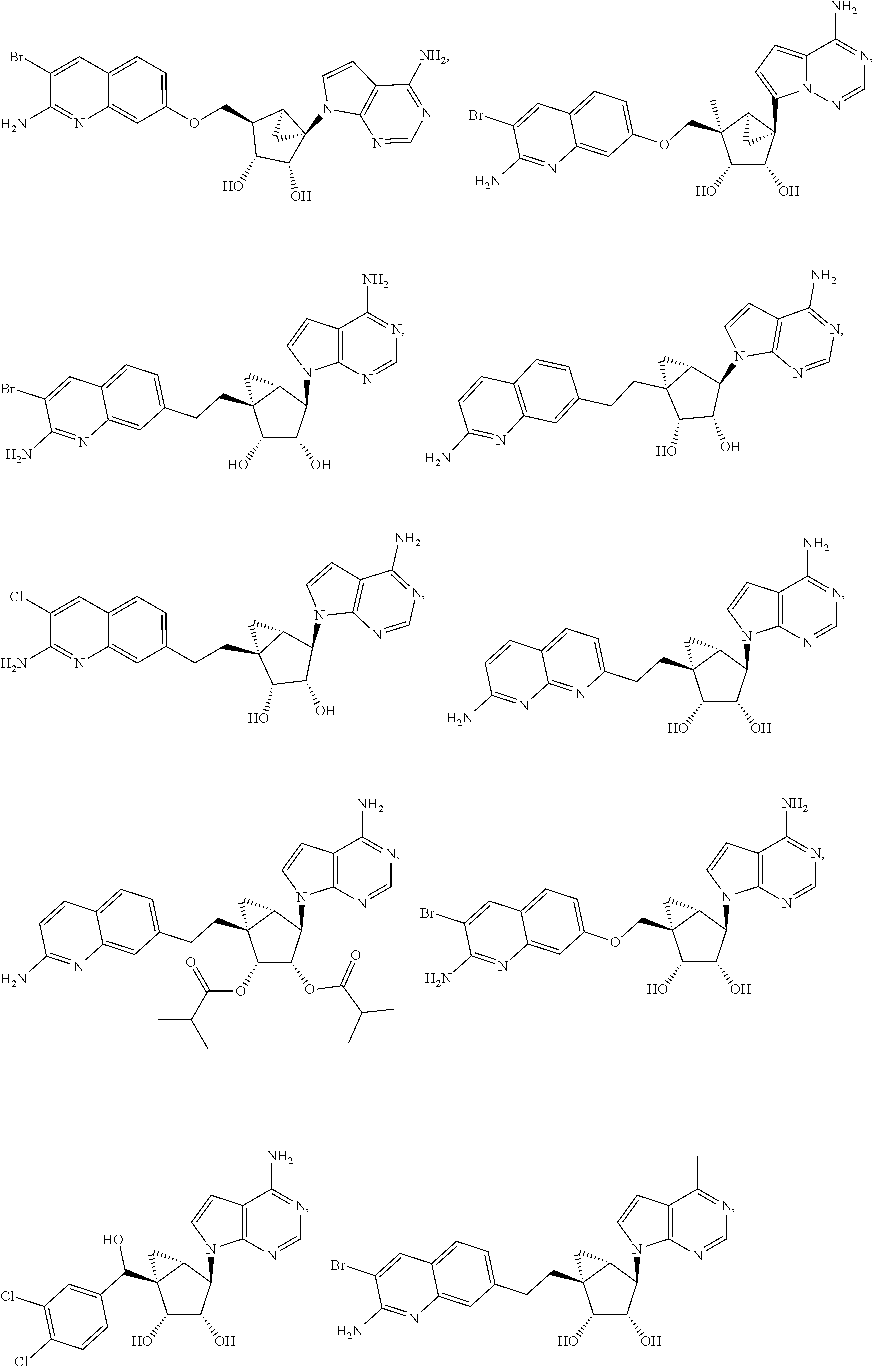

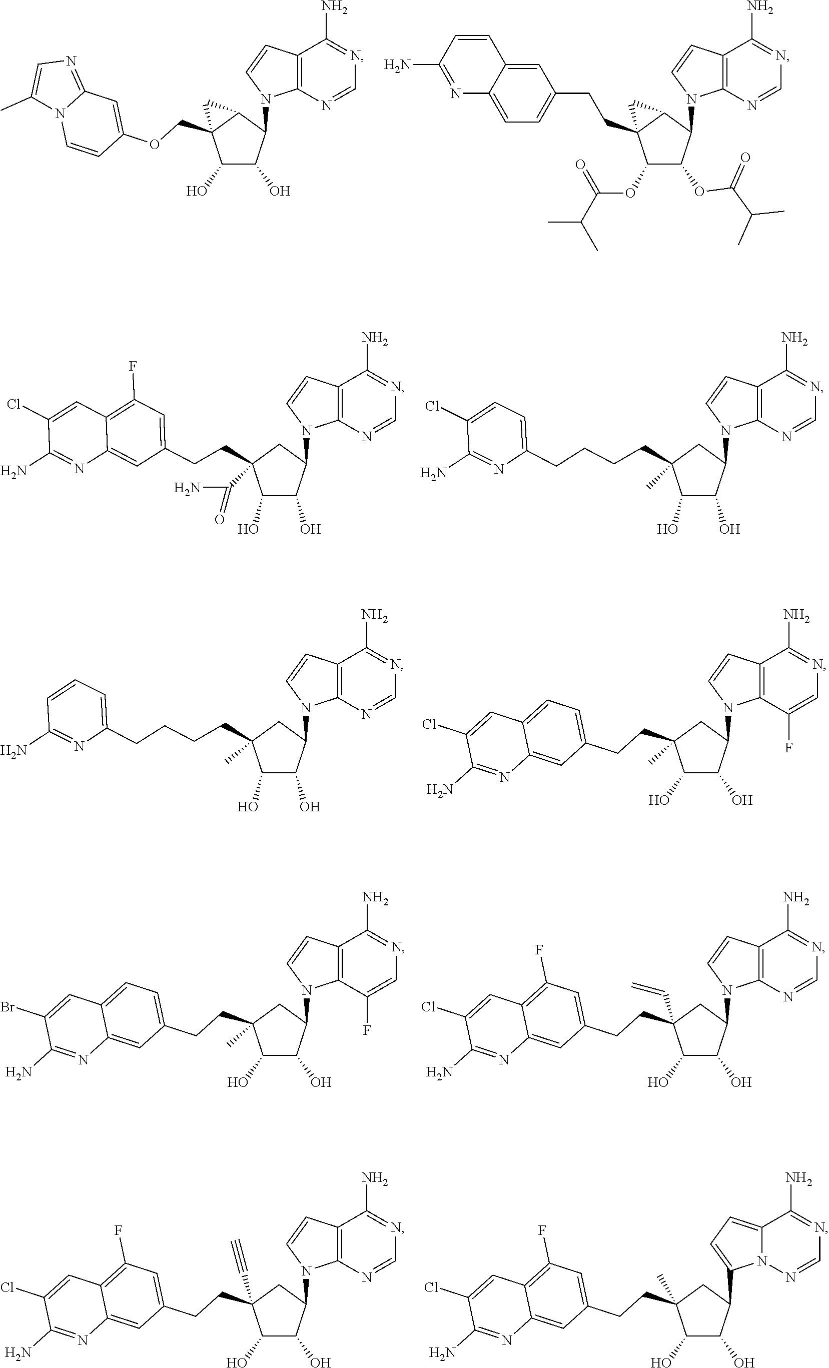

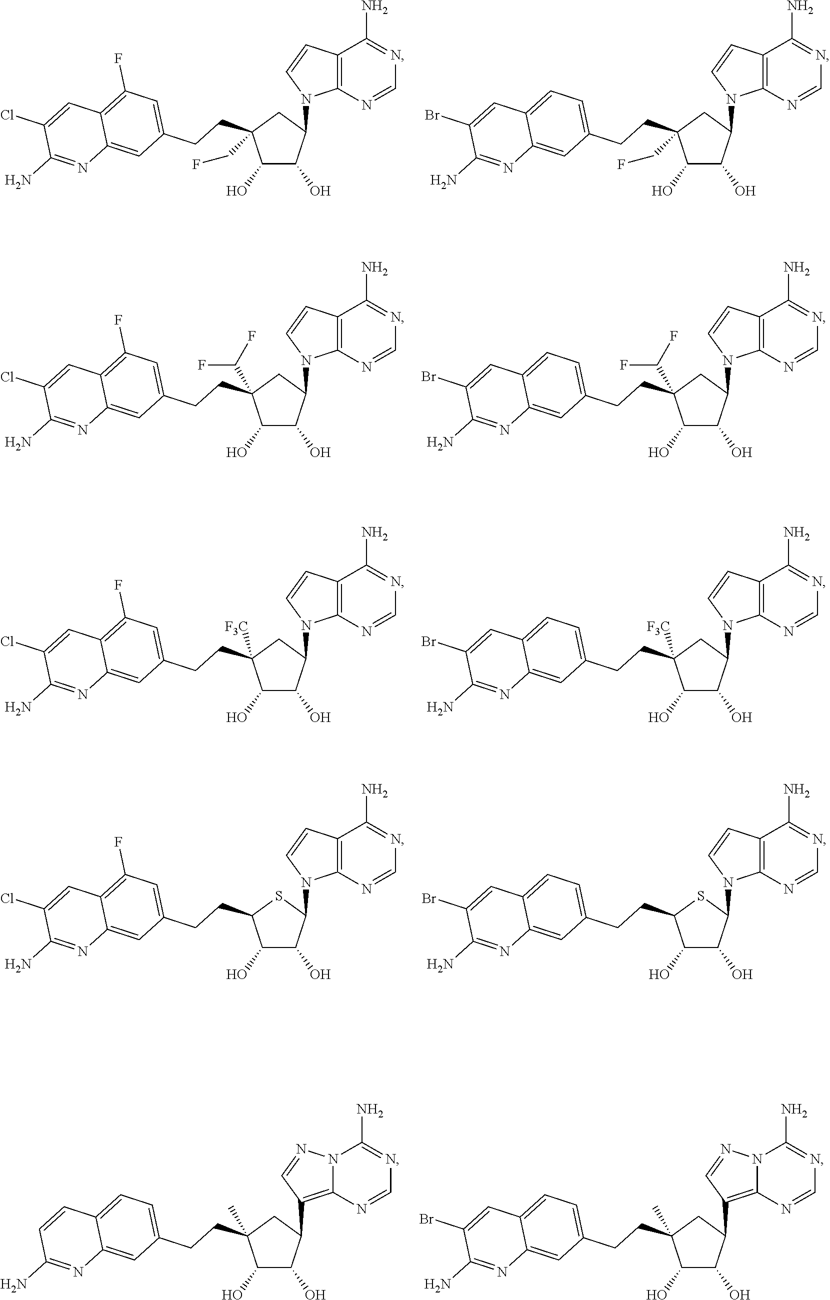

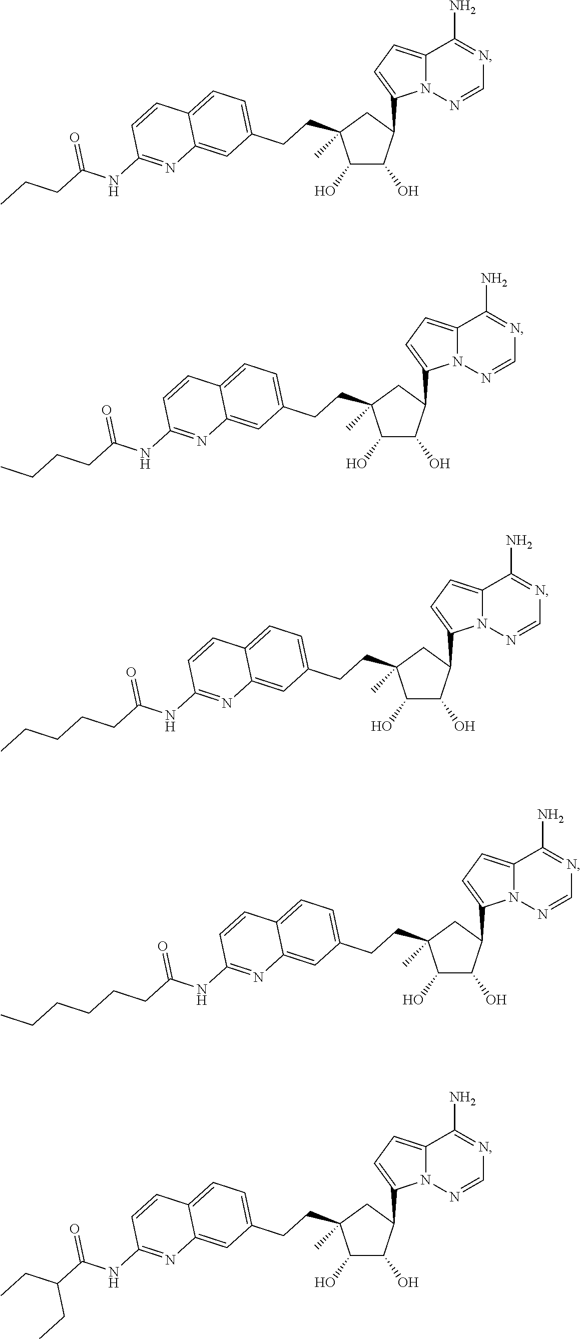 [SMILES: CCCC(=O)Nc1ccc2ccc(CC[C@@]3(C)C[C@@H](c4ccc5c(N)ncnn45)[C@H](O)[C@@H]3O)cc2n1 ; CCCCC(=O)Nc1ccc2ccc(CC[C@@]3(C)C[C@@H](c4ccc5c(N)ncnn45)[C@H](O)[C@@H]3O)cc2n1 ; CCCCCC(=O)Nc1ccc2ccc(CC[C@@]3(C)C[C@@H](c4ccc5c(N)ncnn45)[C@H](O)[C@@H]3O)cc2n1 ; CCCCCCC(=O)Nc1ccc2ccc(CC[C@@]3(C)C[C@@H](c4ccc5c(N)ncnn45)[C@H](O)[C@@H]3O)cc2n1 ; CCC(CC)C(=O)Nc1ccc2ccc(CC[C@@]3(C)C[C@@H](c4ccc5c(N)ncnn45)[C@H](O)[C@@H]3O)cc2n1]

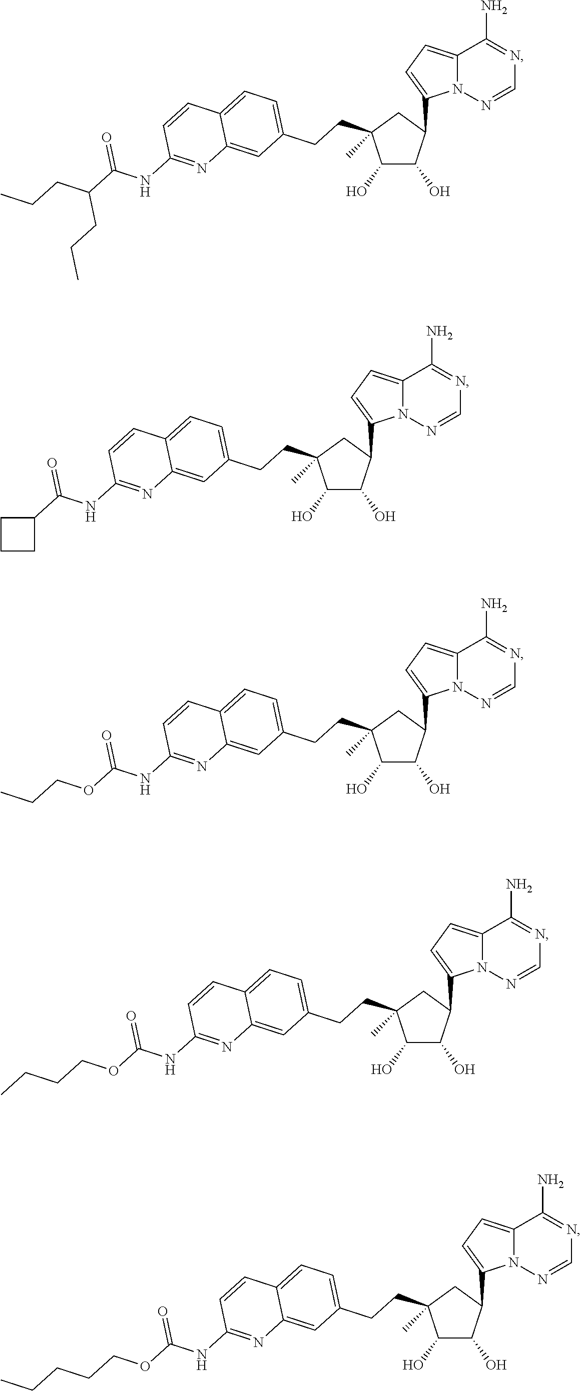 [SMILES: CCCC(CCC)C(=O)Nc1ccc2ccc(CC[C@@]3(C)C[C@@H](c4ccc5c(N)ncnn45)[C@H](O)[C@@H]3O)cc2n1 ; C[C@]1(CCc2ccc3ccc(NC(=O)C4CCC4)nc3c2)C[C@@H](c2ccc3c(N)ncnn23)[C@H](O)[C@@H]1O ; CCCOC(=O)Nc1ccc2ccc(CC[C@@]3(C)C[C@@H](c4ccc5c(N)ncnn45)[C@H](O)[C@@H]3O)cc2n1 ; CCCCOC(=O)Nc1ccc2ccc(CC[C@@]3(C)C[C@@H](c4ccc5c(N)ncnn45)[C@H](O)[C@@H]3O)cc2n1 ; CCCCCOC(=O)Nc1ccc2ccc(CC[C@@]3(C)C[C@@H](c4ccc5c(N)ncnn45)[C@H](O)[C@@H]3O)cc2n1]

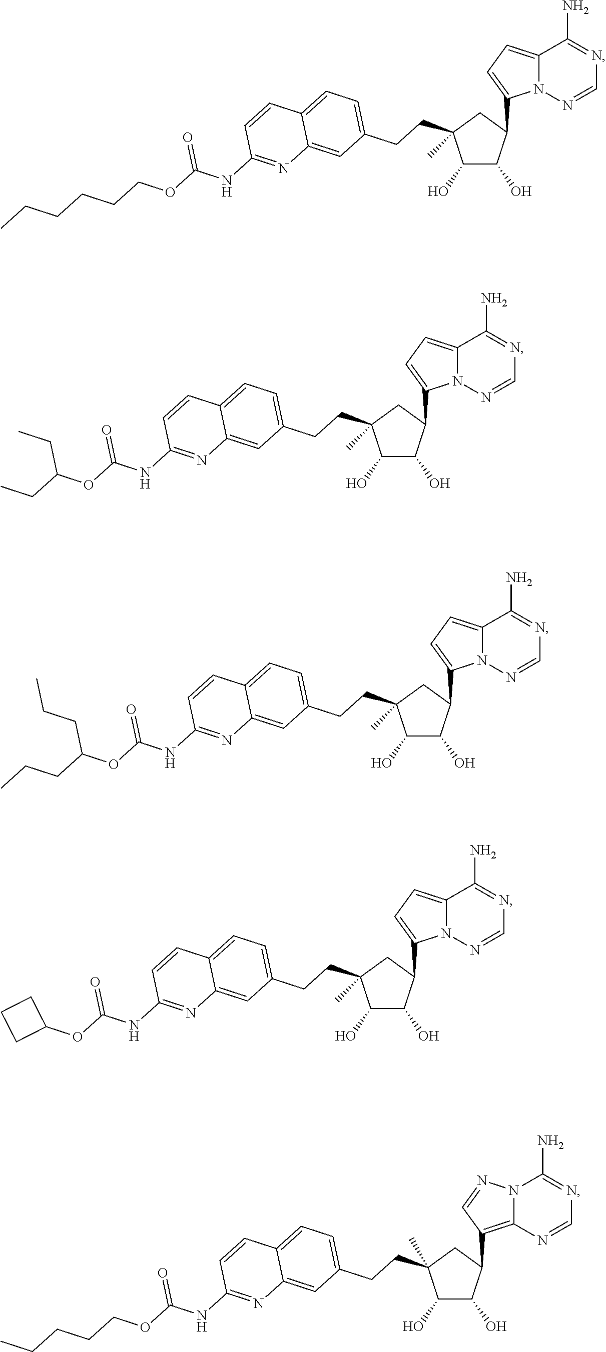 [SMILES: CCCCCCOC(=O)Nc1ccc2ccc(CC[C@@]3(C)C[C@@H](c4ccc5c(N)ncnn45)[C@H](O)[C@@H]3O)cc2n1 ; CCC(CC)OC(=O)Nc1ccc2ccc(CC[C@@]3(C)C[C@@H](c4ccc5c(N)ncnn45)[C@H](O)[C@@H]3O)cc2n1 ; CCCC(CCC)OC(=O)Nc1ccc2ccc(CC[C@@]3(C)C[C@@H](c4ccc5c(N)ncnn45)[C@H](O)[C@@H]3O)cc2n1 ; C[C@]1(CCc2ccc3ccc(NC(=O)OC4CCC4)nc3c2)C[C@@H](c2ccc3c(N)ncnn23)[C@H](O)[C@@H]1O ; CCCCCOC(=O)Nc1ccc2ccc(CC[C@@]3(C)C[C@@H](c4cnn5c(N)ncnc45)[C@H](O)[C@@H]3O)cc2n1]

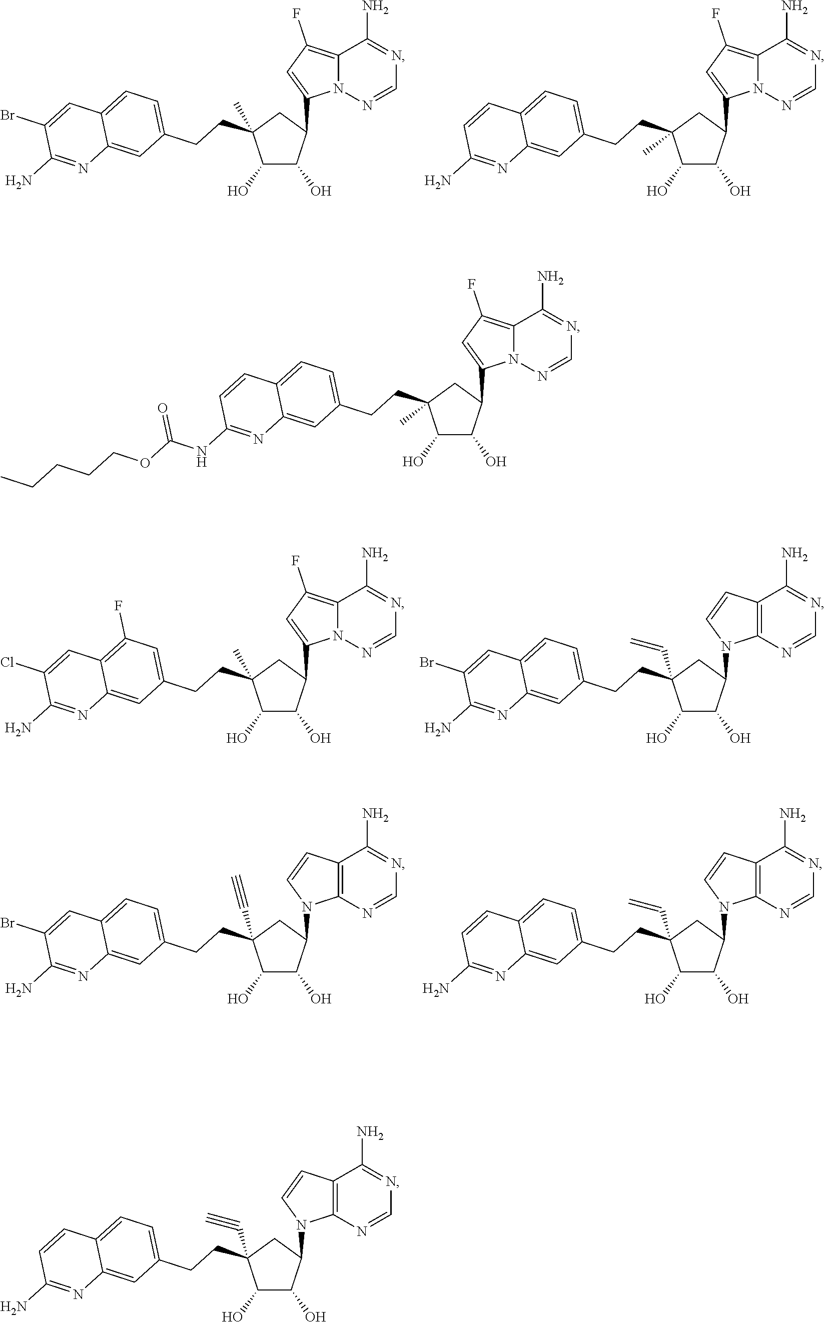

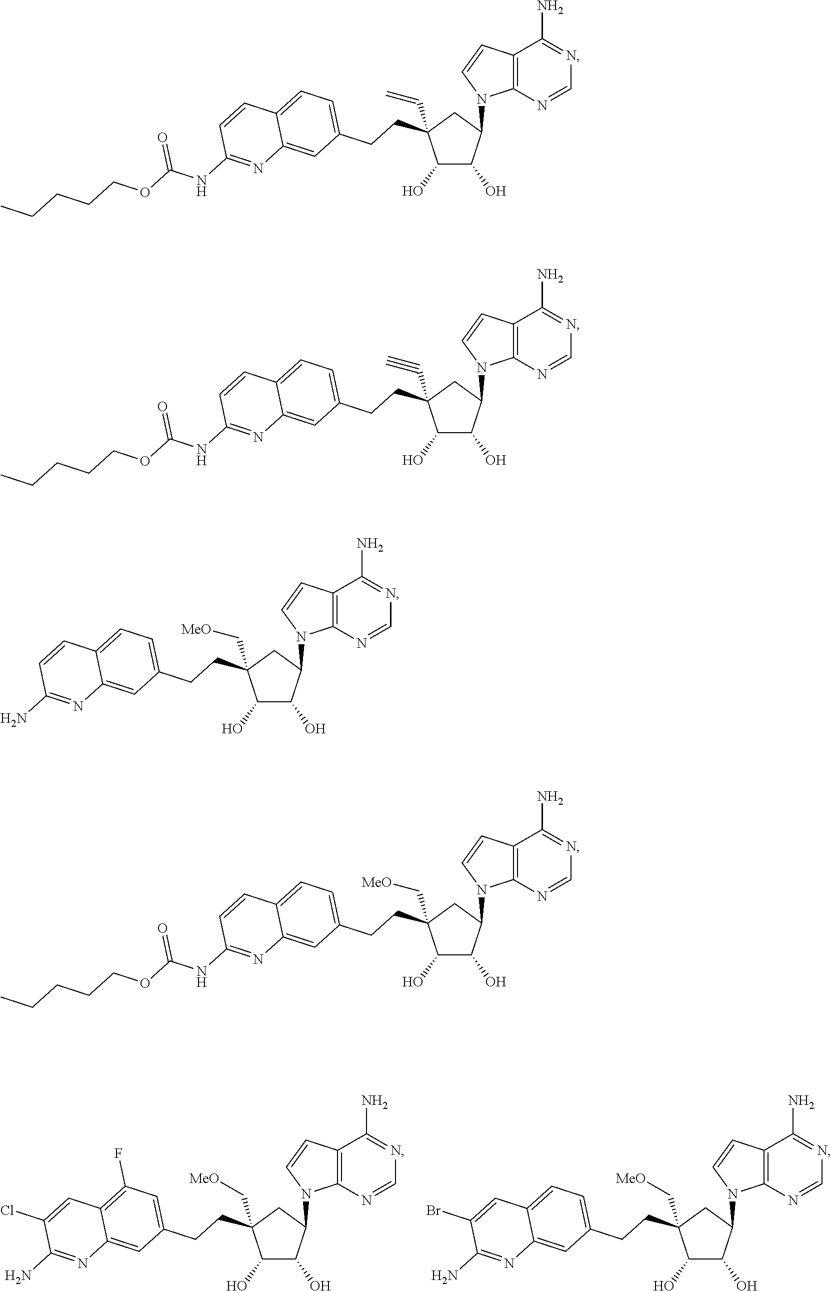 [SMILES: C=C[C@]1(CCc2ccc3ccc(NC(=O)OCCCCC)nc3c2)C[C@@H](n2ccc3c(N)ncnc32)[C@H](O)[C@@H]1O ; C#C[C@]1(CCc2ccc3ccc(NC(=O)OCCCCC)nc3c2)C[C@@H](n2ccc3c(N)ncnc32)[C@H](O)[C@@H]1O ; COC[C@]1(CCc2ccc3ccc(N)nc3c2)C[C@@H](n2ccc3c(N)ncnc32)[C@H](O)[C@@H]1O ; CCCCCOC(=O)Nc1ccc2ccc(CC[C@@]3(COC)C[C@@H](n4ccc5c(N)ncnc54)[C@H](O)[C@@H]3O)cc2n1 ; COC[C@]1(CCc2cc(F)c3cc(Cl)c(N)nc3c2)C[C@@H](n2ccc3c(N)ncnc32)[C@H](O)[C@@H]1O ; COC[C@]1(CCc2ccc3cc(Br)c(N)nc3c2)C[C@@H](n2ccc3c(N)ncnc32)[C@H](O)[C@@H]1O]

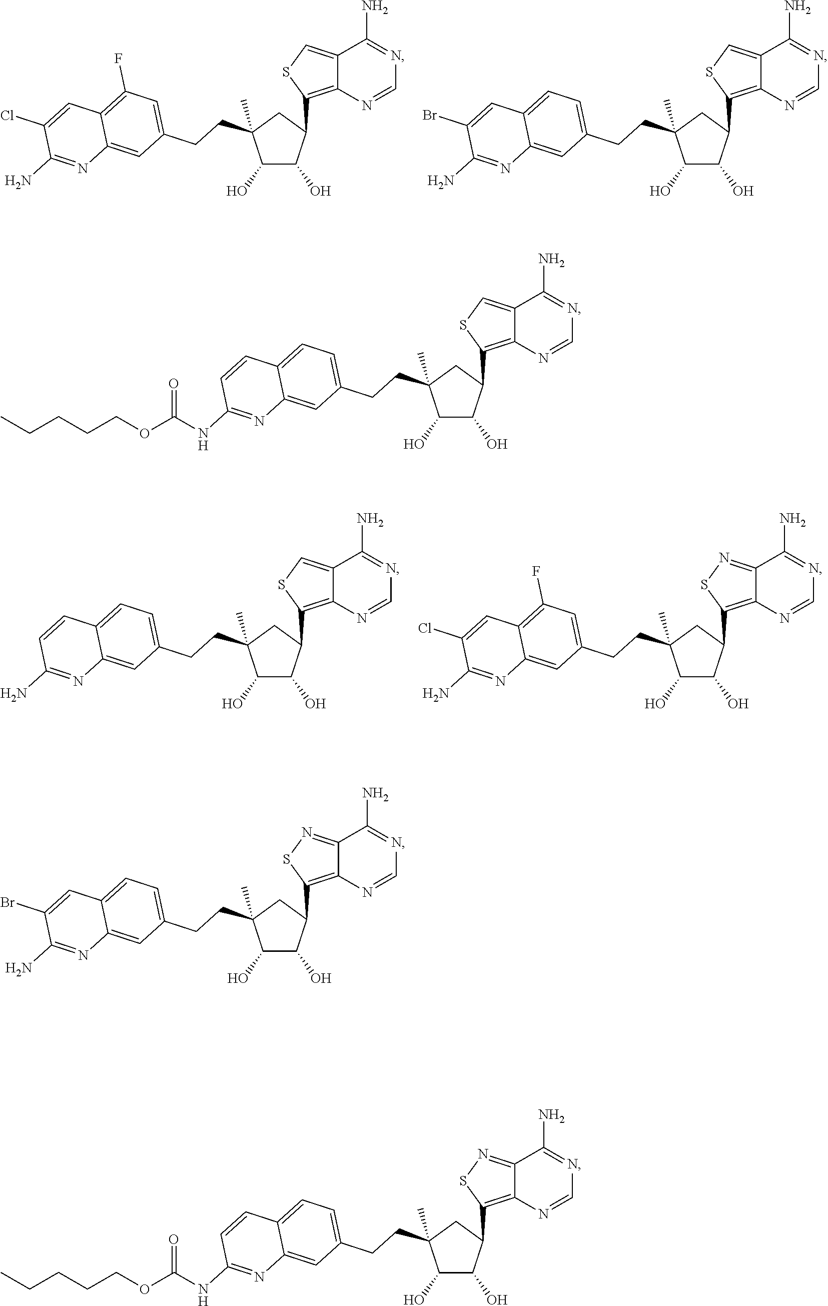

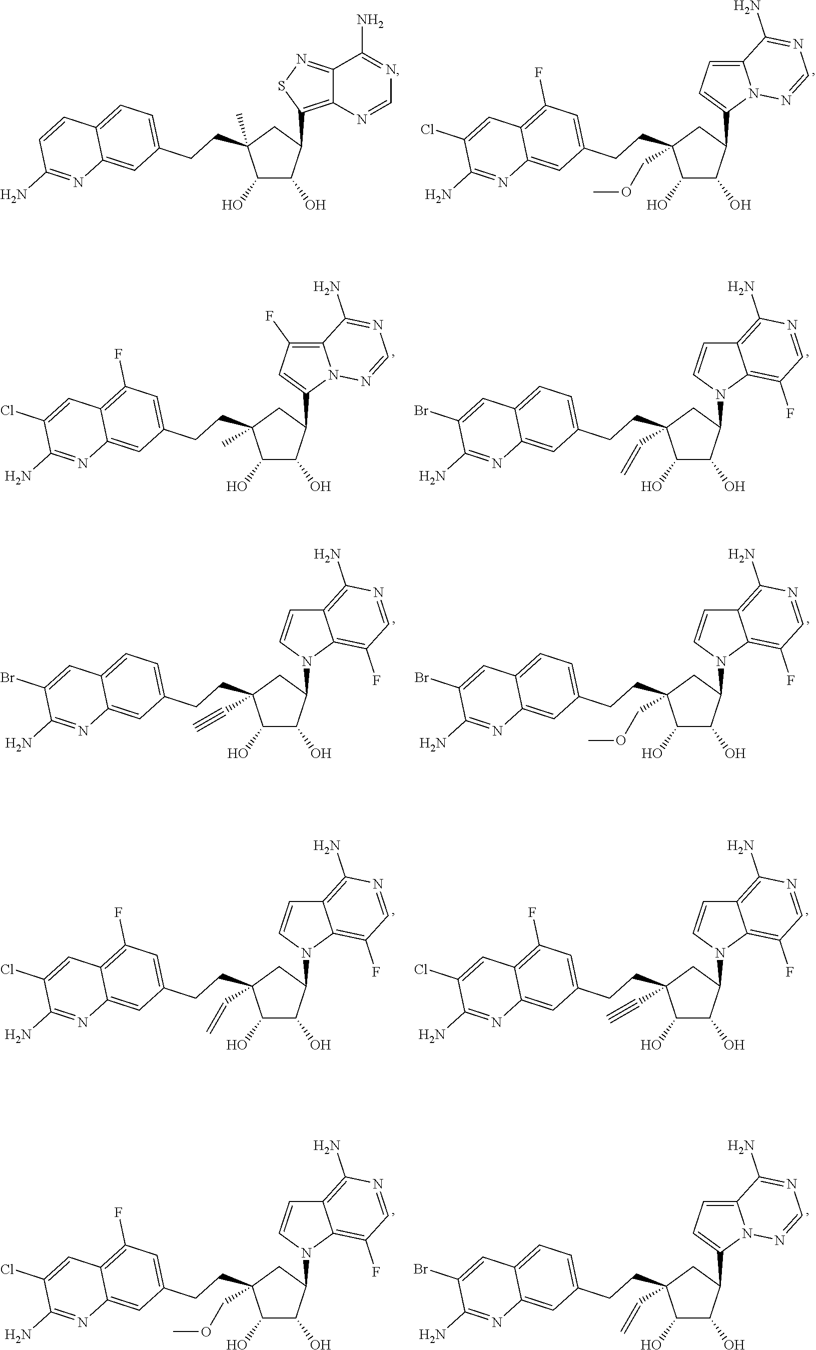

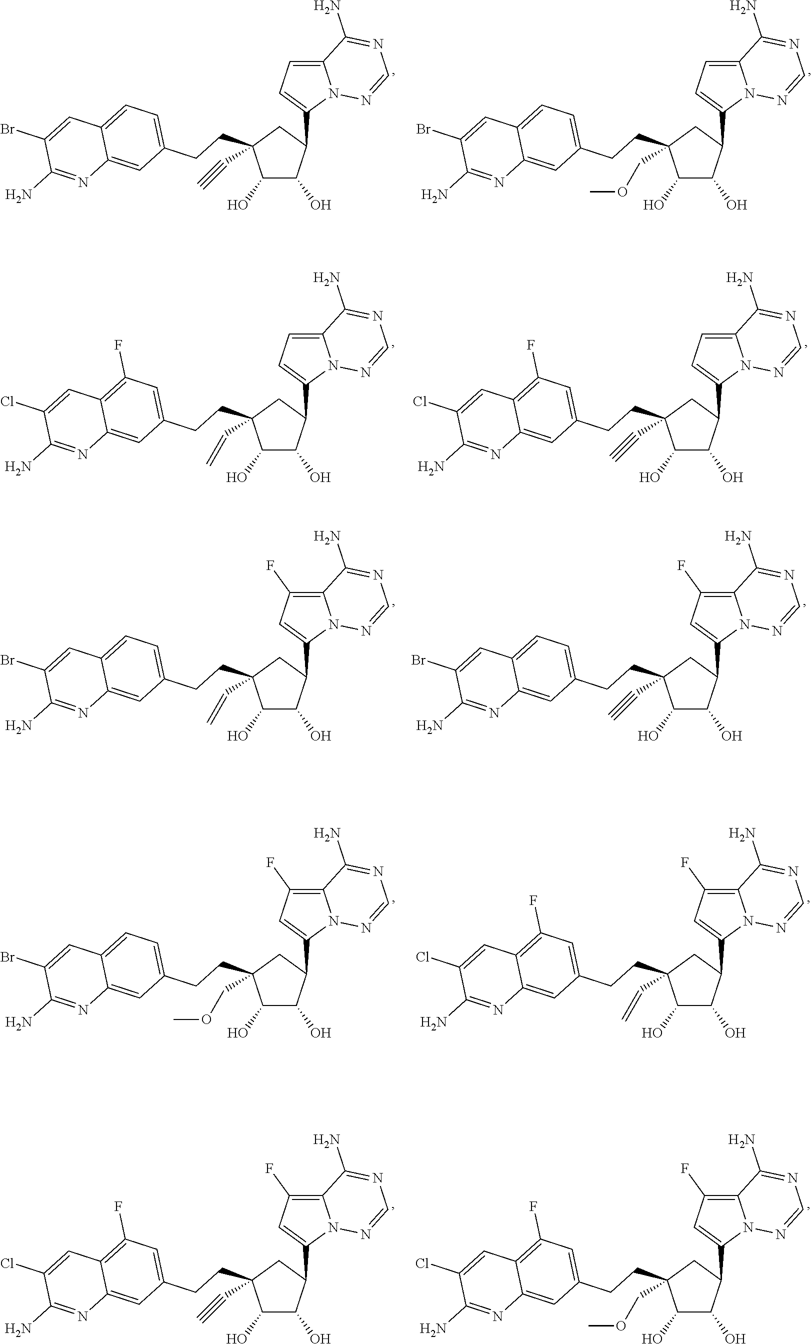

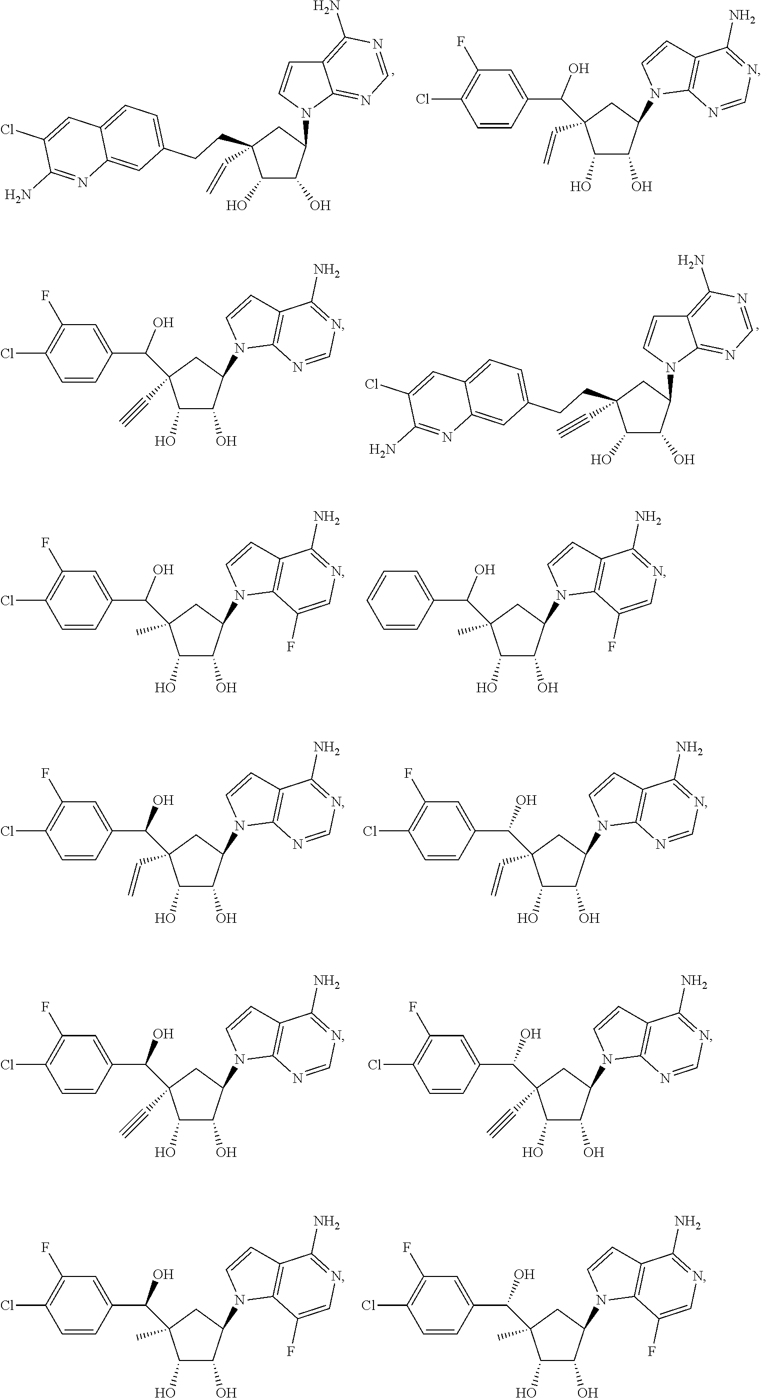

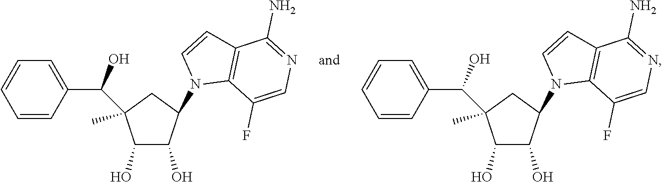 [SMILES: C[C@]1([C@H](O)c2ccccc2)C[C@@H](n2ccc3c(N)ncc(F)c32)[C@H](O)[C@@H]1O ; C[C@]1([C@@H](O)c2ccccc2)C[C@@H](n2ccc3c(N)ncc(F)c32)[C@H](O)[C@@H]1O]

or a pharmaceutically acceptable salt of any of the foregoing, or a pharmaceutically acceptable salt of any of the foregoing.

In some embodiments, R^{F1} cannot be an optionally substituted imidazo[1,2-a]pyridine, an optionally substituted 1H-benzo[d]imidazole, an optionally substituted benzo[d]thiazole, an optionally substituted 1H-pyrrolo[3,2-b]pyridine, an optionally substituted thieno[3,2-b]pyridine, an optionally substituted furo[3,2-b]pyridine, an optionally substituted 1H-pyrrolo[2,3-b]pyridine, an optionally substituted 1H-pyrazole, an optionally substituted pyrimidine, an optionally substituted 1,8a-dihydroimidazo[1,2-a]pyridin-2(3H)-one, an optionally substituted 3,4-dihydro-2H-pyrido[3,2-b][1,4]oxazine, an optionally substituted 2,3-dihydro-1H-pyrrolo[2,3-b]pyridine, an optionally substituted 1H-imidazole and/or an optionally substituted 1H-pyrrolo[2,3-c]pyridine. In some embodiments, when R¹, R^{2A}, R^{3A}, R^{4B}, R^{5A} and R^{5B} are each H; R^{2B} and R^{3B} are each OH; and R^{4A} is —CH₂—R^{F1} or —(CH₂)₂—R^{F1}, then R^{F1} cannot be an optionally substituted imidazo[1,2-a]pyridine, an optionally substituted 1H-benzo[d]imidazole, an optionally substituted benzo[d]thiazole, an optionally substituted 1H-pyrrolo[3,2-b]pyridine, an optionally substituted thieno[3,2-b]pyridine, an optionally substituted furo[3,2-b]pyridine, an optionally substituted 1H-pyrrolo[2,3-b]pyridine, an optionally substituted 1H-pyrazole, an optionally substituted pyrimidine, an optionally substituted 1,8a-dihydroimidazo[1,2-a]pyridin-2(3H)-one, an optionally substituted 3,4-dihydro-2H-pyrido[3,2-b][1,4]oxazine, an optionally substituted 2,3-dihydro-1H-pyrrolo[2,3-b]pyridine, an optionally substituted 1H-imidazole and/or an optionally substituted 1H-pyrrolo[2,3-c]pyridine. In some embodiments, when R¹, R^{2A}, R^{3A}, R^{4B}, R^{5A} and R^{5B} are each H; R^{2B} and R^{3B} are each OH; and R^{4A} is —CH₂—O—R^{J1}, then R^{J1} cannot be an optionally substituted imidazo[1,2-a]pyridine, an optionally substituted 1H-benzo[d]imidazole, an optionally substituted benzo[d]thiazole, an optionally substituted 1H-pyrrolo[3,2-b]pyridine, an optionally substituted thieno[3,2-b]pyridine, an optionally substituted furo[3,2-b]pyridine, an optionally substituted 1H-pyrrolo[2,3-b]pyridine, an optionally substituted 1H-pyrazole, an optionally substituted pyrimidine, an optionally substituted 1,8a-dihydroimidazo[1,2-a]pyridin-2(3H)-one, an optionally substituted 3,4-dihydro-2H-pyrido[3,2-b][1,4]oxazine, an optionally substituted 2,3-dihydro-1H-pyrrolo[2,3-b]pyridine, an optionally substituted 1H-imidazole and/or an optionally substituted 1H-pyrrolo[2,3-c]pyridine. In some embodiments, when B¹ is

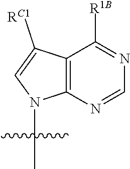

(such as

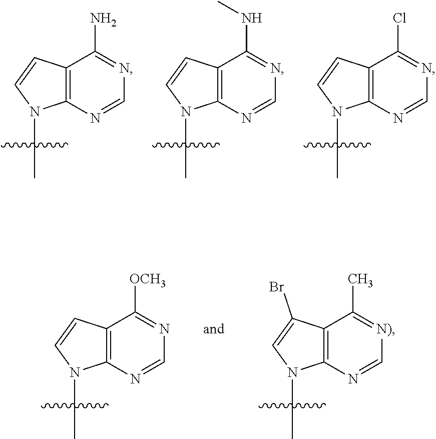

then R^{F1} cannot be an optionally substituted imidazo[1,2-a]pyridine, an optionally substituted 1H-benzo[d]imidazole, an optionally substituted benzo[d]thiazole, an optionally substituted 1H-pyrrolo[3,2-b]pyridine, an optionally substituted thieno[3,2-b]pyridine, an optionally substituted furo[3,2-b]pyridine, an optionally substituted 1H-pyrrolo[2,3-b]pyridine, an optionally substituted 1H-pyrazole, an optionally substituted pyrimidine, an optionally substituted 1,8a-dihydroimidazo[1,2-a]pyridin-2(3H)-one, an optionally substituted 3,4-dihydro-2H-pyrido[3,2-b][1,4]oxazine, an optionally substituted 2,3-dihydro-1H-pyrrolo[2,3-b]pyridine, an optionally substituted 1H-imidazole and/or an optionally substituted 1H-pyrrolo[2,3-c]pyridine. In some embodiments, when R¹, R^{2A}, R^{3A}, R^{4B}, R^{5A} and R^{5B} are each H; R^{2B} and R^{3B} are each OH; R^{4A} is —CH₂—R^{F1}, —(CH₂)₂—R^{F1} or —CH₂—O—R^{J1}; and B¹ is

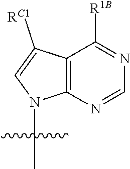

(such as

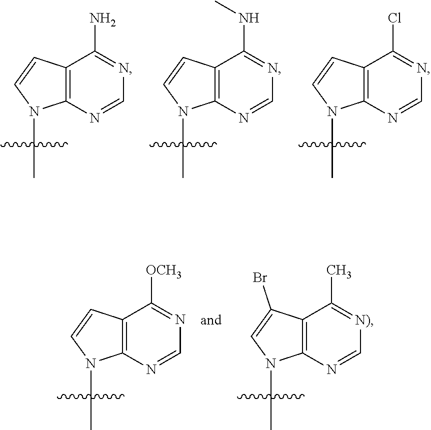

then R^{F1} and/or R^{J1} cannot be an optionally substituted imidazo[1,2-a]pyridine, an optionally substituted 1H-benzo[d]imidazole, an optionally substituted benzo[d]thiazole, an optionally substituted 1H-pyrrolo[3,2-b]pyridine, an optionally substituted thieno[3,2-b]pyridine, an optionally substituted furo[3,2-b]pyridine, an optionally substituted 1H-pyrrolo[2,3-b]pyridine, an optionally substituted 1H-pyrazole, an optionally substituted pyrimidine, an optionally substituted 1,8a-dihydroimidazo[1,2-a]pyridin-2(3H)-one, an optionally substituted 3,4-dihydro-2H-pyrido[3,2-b][1,4]oxazine, an optionally substituted 2,3-dihydro-1H-pyrrolo[2,3-b]pyridine, an optionally substituted 1H-imidazole and/or an optionally substituted 1H-pyrrolo[2,3-c]pyridine.

In some embodiments, R^{F1} cannot be an optionally substituted quinoline. In some embodiments, R^{F1} cannot be an optionally substituted quinazoline. In some embodiments, R^{F1} cannot be an optionally substituted quinoxaline. In some embodiments, when R¹, R^{2A}, R^{3A}, R^{4B}, R^{5A} and R^{5B} are each H; R^{2B} and R^{3B} are each OH; and R^{4A} is —(CH₂)₂—R^{F1}, then R^{F1} cannot be an optionally substituted quinolone, an optionally substituted quinazoline and/or an optionally substituted quinoxaline. In some embodiments, when B¹ is

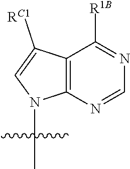

(such as

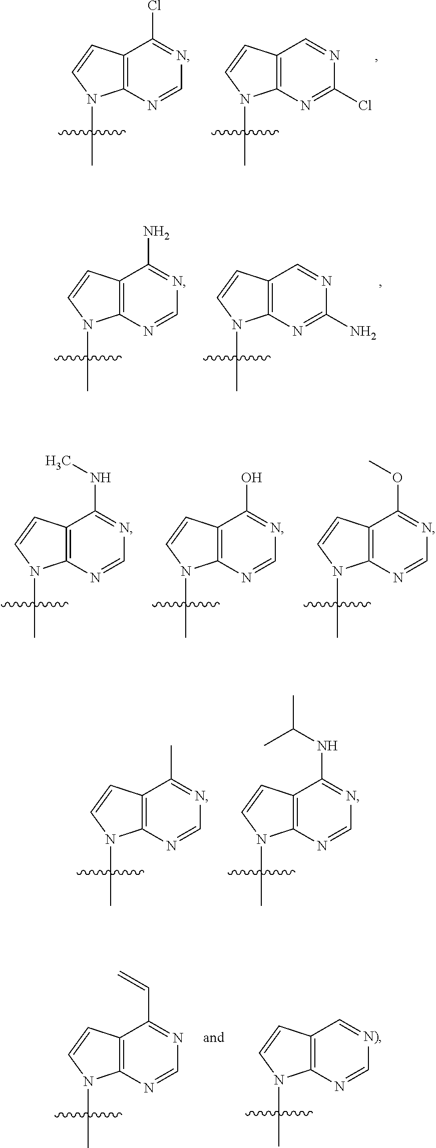

then R^{F1} cannot be an optionally substituted quinolone, an optionally substituted quinazoline and/or an optionally substituted quinoxaline. In some embodiments, when R¹, R^{2A}, R^{3A}, R^{4B}, R^{5A} and R^{5B} are each H; R^{2B} and R^{3B} are each OH; R^{4A} is —(CH₂)₂—R^{F1}; and B¹ is

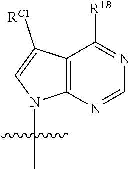

(such as

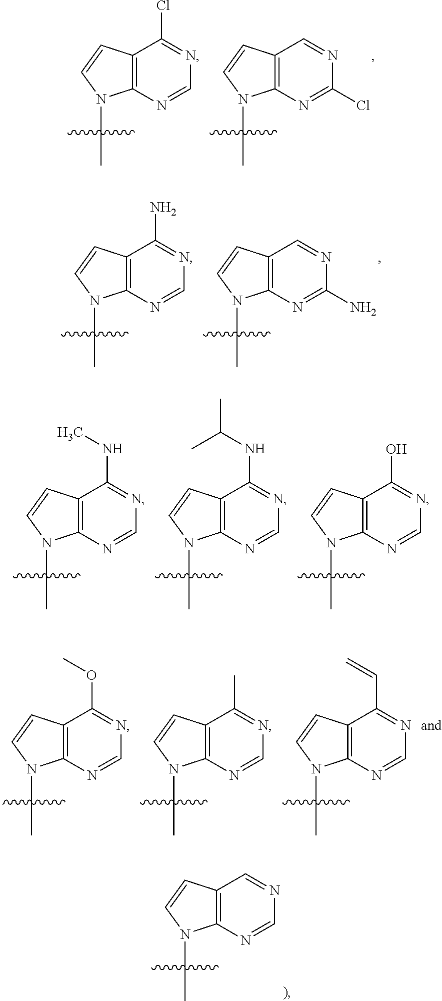

then R^{F1} cannot be an optionally substituted quinoline, an optionally substituted quinazoline and/or an optionally substituted quinoxaline. In some embodiments, when R¹, R^{2A}, R^{3A}, R^{4B}, R^{5A} and R^{5B} are each H; R^{2B} and R^{3B} are each OH; R^{4A} is —(CH₂)₂—R^{F1}; and B¹ is

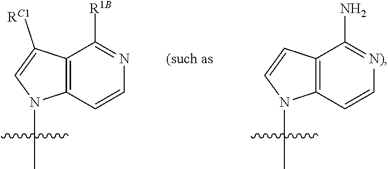

then R^{F1} cannot be an optionally substituted quinolone, an optionally substituted quinazoline and/or an optionally substituted quinoxaline. In some embodiments, when R¹, R^{2A}, R^{3A}, R^{4B}, R^{5A} and R^{5B} are each H; R^{2B} and R^{3B} are each OH; R^{4A} is —O—(CH₂)—R^{M1}; and B¹ is

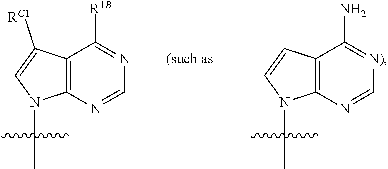

then R^{M1} cannot be an optionally substituted quinolone, an optionally substituted quinazoline and/or an optionally substituted quinoxaline. In some embodiments, when R¹, R^{2A}, R^{3A}, R^{4B}, R^{5A} and R^{5B} are each H; R^{2B} and R^{3B} are each OH; R^{4A} is-CH₂—R^{F1}; and B¹ is

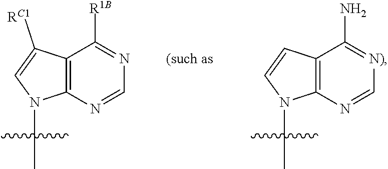

then R^{F1} cannot be an optionally substituted quinolone, an optionally substituted quinazoline and/or an optionally substituted quinoxaline. In some embodiments, when R¹, R^{2A}, R^{3A}, R^{4B}, R^{5A} and R^{5B} are each H; R^{2B} and R^{3B} are each OH; R^{4A} is —CH₂—R^{F1}; and B¹ is

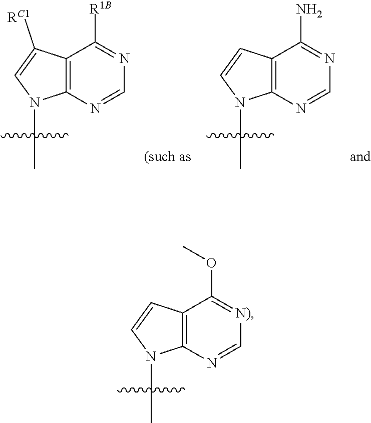

then R^{F1} cannot be an optionally substituted naphthalene. In some embodiments, when R¹, R^{2A}, R^{3A}, R^{4B}, R^{5A} and R^{5B} are each H; R^{2B} and R^{3B} are each OH; R^{4A} is —CH₂—O—R^{J1}; and B¹ is

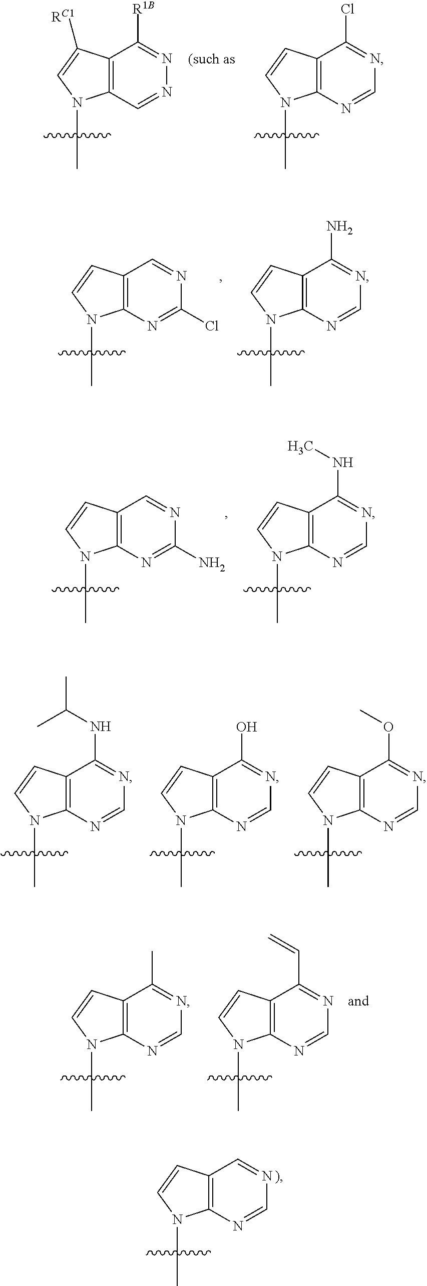

then R^{J1} cannot be an optionally substituted quinolone, an optionally substituted quinazoline and/or an optionally substituted quinoxaline.

In some embodiments, R^{F1} cannot be an optionally substituted phenyl, an optionally substituted thiophene, an optionally substituted pyridine and/or an optionally substituted 1,2,3,4-tetrahydroisoquinoline. In some embodiments, when R¹, R^{2A}, R^{3A}, R^{4B}, R^{5A} and R^{5B} are each H; R^{2B} and R^{3B} are each OH; and R^{4A} is —CH₂—R^{F1}, —CH(OH)—R^{F1}, —CH(F)—R^{F1} or —CH(OH)—CH₂—R^{F1}, then R^{F1} cannot be an optionally substituted phenyl, an optionally substituted thiophene, an optionally substituted pyridine and/or an optionally substituted 1,2,3,4-tetrahydroisoquinoline. In some embodiments, when R¹, R^{2A}, R^{3A}, R^{4B}, R^{5A} and R^{5B} are each H; R^{2B} and R^{3B} are each OH; R^{4A} is —CH₂—R^{F1}, —CH(OH)—R^{F1}, —CH(F)—R^{F1} or —CH(OH)—CH₂—R^{F1}; and B¹ is

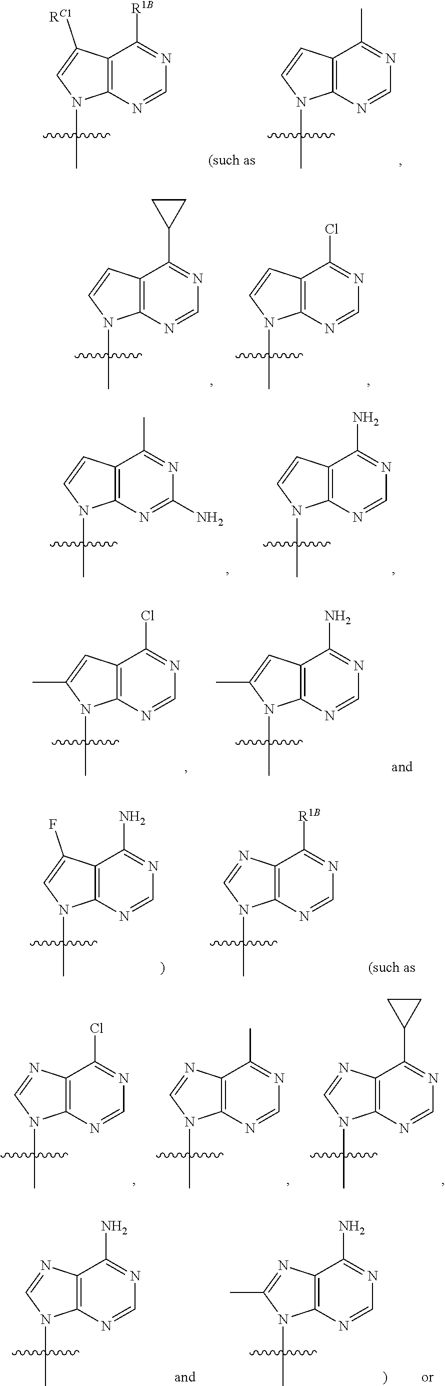

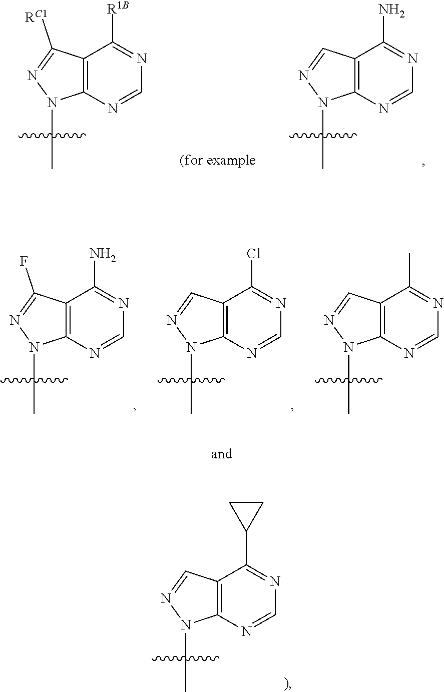

then R^{F1} cannot be an optionally substituted phenyl, an optionally substituted thiophene, an optionally substituted pyridine and/or an optionally substituted 1,2,3,4-tetrahydroisoquinoline. In some embodiments, R^{4A} cannot be —CH(OH)—R^{F1}.

In some embodiments, B¹ cannot be an optionally substituted

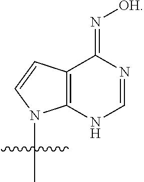 [SMILES: *n1ccc2/c(=N/O)nc[nH]c21]

In some embodiments, B¹ cannot be an optionally substituted

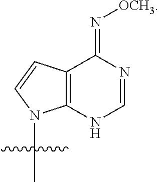 [SMILES: *n1ccc2/c(=N/OC)nc[nH]c21]

In some embodiments, when R¹, R^{2A}, R^{3A}, R^{4B}, R^{5A} and R^{5B} are each H; R^{2B} and R^{3B} are each OH; R^{4A} is —CH(OH)—R^{F1}; R^{F1} is an optionally substituted phenyl; then B¹ cannot be an optionally substituted

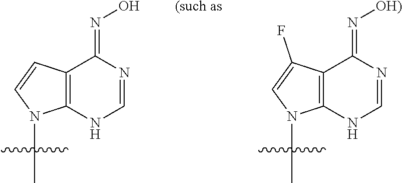

or an optionally substituted

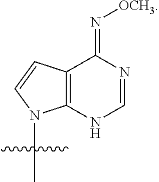 [SMILES: *n1ccc2/c(=N/OC)nc[nH]c21]

In some embodiments, when R¹, R^{2A}, R^{3A}, R^{4B}, R^{5A} and R^{5B} are each H; R^{2B} and R^{3B} are each OH; R^{4A} is —(CH₂)₂—R^{F1} or —CH₂—O—R^{J1}; R^{F1} and/or R^{J1} is an optionally substituted quinoline; then B¹ cannot be an optionally substituted

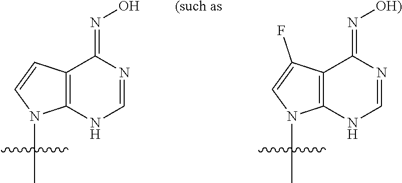

or an optionally substituted

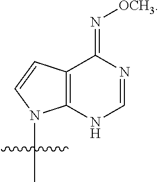 [SMILES: *n1ccc2/c(=N/OC)nc[nH]c21]

In some embodiments, B¹ cannot be

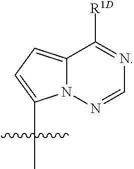

In some embodiments, B¹ cannot be one or more of the following:

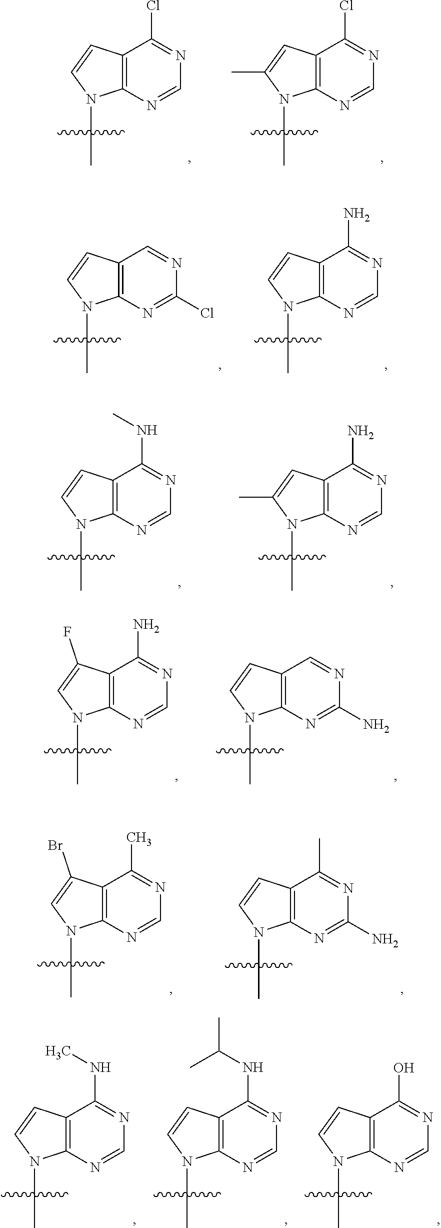

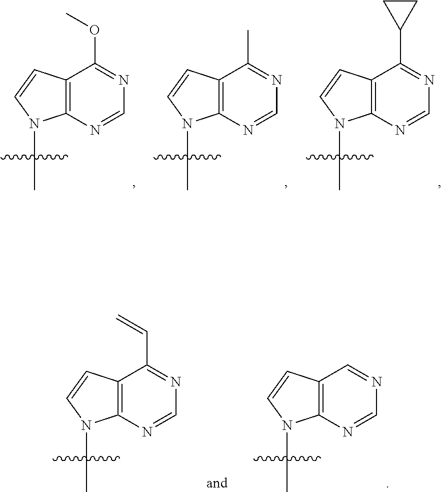

In some embodiments, B¹ cannot be one or more of the following:

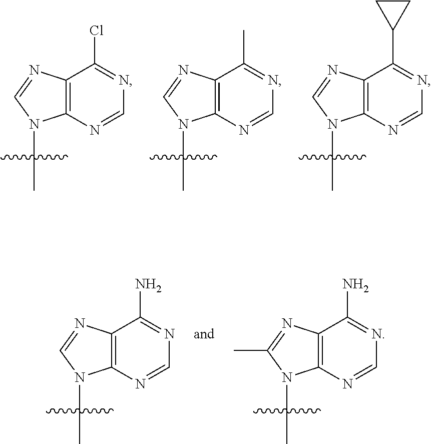

In some embodiments, B¹ cannot be one or more of the following:

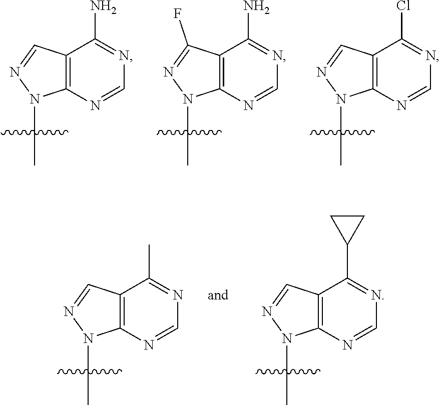 [SMILES: *n1ncc2c(N)ncnc21 ; *n1nc(F)c2c(N)ncnc21 ; *n1ncc2c(Cl)ncnc21 ; *n1ncc2c(C)ncnc21 ; "and" ; *n1ncc2c(C3CC3)ncnc21]

In some embodiments, when R¹, R^{2A}, R^{3A}, R^{4B}, R^{5A} and R^{5B} are each H; R^{2B} and R^{3B} are each OH; R^{4A} is —CH(OH)—R^{F1}, —(CH₂)₁₋₂—R^{F1}, —CH(F)—R^{F1}, —CH(OH)—CH₂—R^{F1}, —CH₂—O—R^{J1} or —O—CH₂—R^{M1}; then R^{F1}, R^{J1} and/or R^{M1} cannot be an optionally substituted phenyl, an optionally substituted naphthalene, an optionally substituted pyridine, an optionally substituted 1,2,3,4-tetrahydroisoquinoline, an optionally substituted quinoline, an optionally substituted quinazoline, an optionally substituted quinoxaline and/or an optionally substituted imidazo[1,2-a]pyridine. In some embodiments, R^{F1}, R^{J1} and/or R^{M1} cannot be an optionally substituted phenyl, an optionally substituted naphthalene, an optionally substituted pyridine, an optionally substituted 1,2,3,4-tetrahydroisoquinoline, an optionally substituted quinoline, an optionally substituted quinazoline, an optionally substituted quinoxaline, an optionally substituted imidazo[1,2-a]pyridine, an optionally substituted 1H-benzo[d]imidazole, an optionally substituted benzo[d]thiazole, an optionally substituted 1H-pyrrolo[3,2-b]pyridine, an optionally substituted thieno[3,2-b]pyridine, an optionally substituted furo[3,2-b]pyridine, an optionally substituted 1H-pyrrolo[2,3-b]pyridine, an optionally substituted 1H-pyrazole, an optionally substituted pyrimidine, an optionally substituted 1,8a-dihydroimidazo[1,2-a]pyridin-2(3H)-one, an optionally substituted 3,4-dihydro-2H-pyrido[3,2-b][1,4]oxazine, an optionally substituted 2,3-dihydro-1H-pyrrolo[2,3-b]pyridine, an optionally substituted 1H-imidazole and/or an optionally substituted 1H-pyrrolo[2,3-c]pyridine. In some embodiments, R^{F1} cannot be

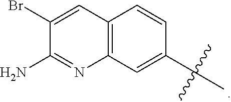 [SMILES: *c1ccc2cc(Br)c(N)nc2c1]

In some embodiments, R^{J1} cannot be

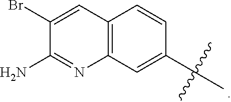 [SMILES: *c1ccc2cc(Br)c(N)nc2c1]

In some embodiments, R^{J1} cannot be

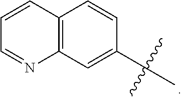 [SMILES: *c1ccc2cccnc2c1]

In some embodiments, R^{J1} cannot be

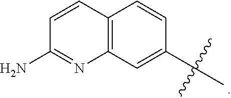 [SMILES: *c1ccc2ccc(N)nc2c1]

In some embodiments, when Z¹ is O, R^{4B} is an unsubstituted C₁₋₄ alkyl (such as methyl), then R^{J1} cannot be

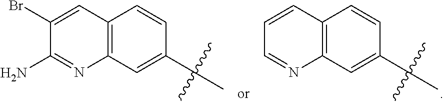 [SMILES: *c1ccc2cc(Br)c(N)nc2c1 ; *c1ccc2cccnc2c1]

In some embodiments, when Z¹ is CH₂, R^{4B} is an unsubstituted C₁₋₄ alkyl (such as methyl), then R^{F1} cannot be

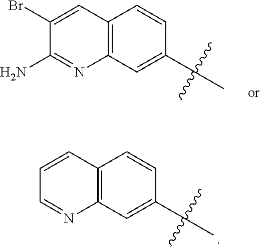 [SMILES: *c1ccc2cc(Br)c(N)nc2c1 ; *c1ccc2cccnc2c1]

In some embodiments, when Z¹ is S, R^{4B} is an unsubstituted C₁₋₄ alkyl (such as methyl), then R^{F1} cannot be

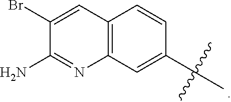 [SMILES: *c1ccc2cc(Br)c(N)nc2c1]

In some embodiments, when R¹, R^{2A}, R^{3A} and R^{5A} are each H; R^{4B} and R^{5B} together with the carbon R^{4B} and R^{5B} are attached form an unsubstituted cyclopropyl; R^{2B} and R^{3B} are each OH; and R^{4A} is —CH₂CH₂—R^{F1}, —CH₂CH(CH₃)—R^{F1}, —CH(CH₃)CH₂—R^{F1} or —CH₂O—R^{J1}; and B¹ is

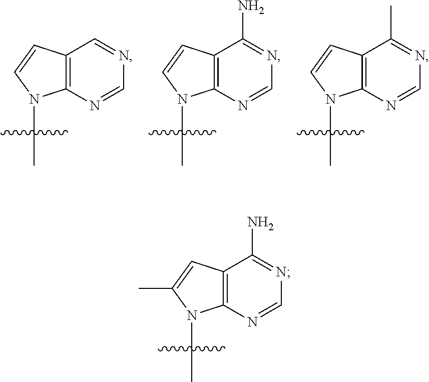

then R^{F1} cannot be

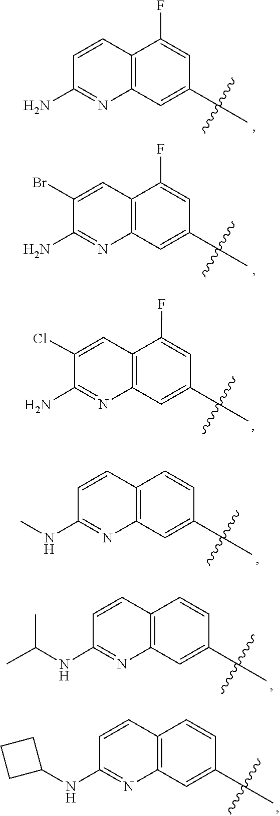

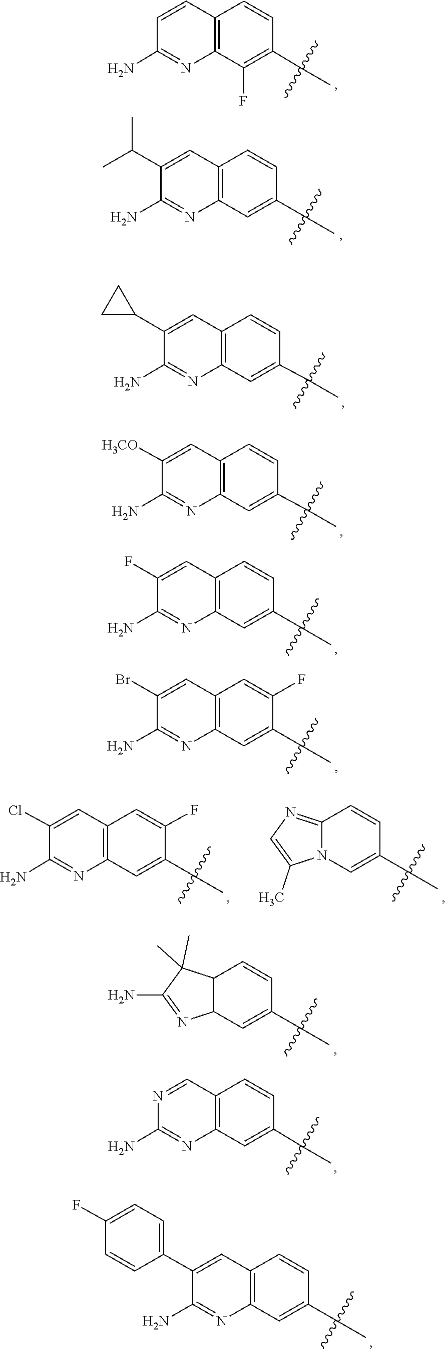

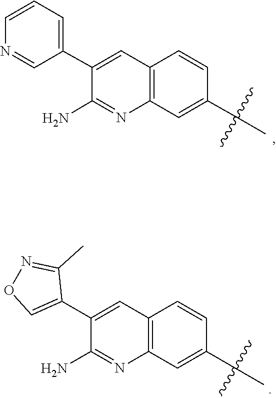 [SMILES: *c1ccc2cc(-c3cccnc3)c(N)nc2c1 ; *c1ccc2cc(-c3conc3C)c(N)nc2c1]

In some embodiments, when R¹, R^{2A}, R^{3A} and R^{5A} are each H; and R^{2B} and R^{3B} are each OH; then R^{4B} and R^{5B} together with the carbon R^{4B} and R^{5B} are attached form an unsubstituted cyclopropyl. In some embodiments, when R¹, R^{2A}, R^{3A} and R^{5A} are each H; R^{4B} and R^{5B} together with the carbon R^{4B} and R^{5B} are attached form an unsubstituted cyclopropyl; R^{2B} and R^{3B} are each OH; and R^{4A} is —CH₂CH₂—R^{F1}, —CH₂CH(CH₃)—R^{F1}, —CH(CH₃)CH₂—R^{F1} or —CH₂O—R^{J1}; and B¹ is

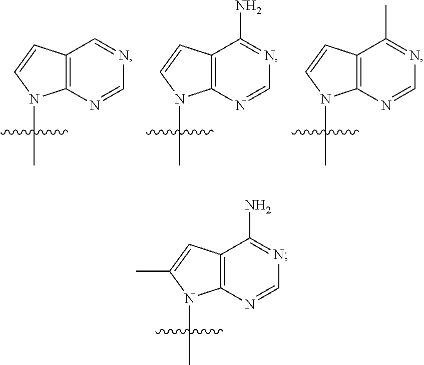

then R^{F1} cannot be an optionally substituted heteroaryl. In some embodiments, R^{4B} and R^{5B} cannot be together with the carbon R^{4B} and R^{5B} are attached form an unsubstituted cyclopropyl.

In some embodiments, R^{4A} cannot be —(CR^{D1}R^{E1})(CR^{D1}R^{E1})ₙ—R^{F1}. In some embodiments, R^{4A} cannot be —CH₂—R^{F1}. In some embodiments, R^{4A} cannot be —(CH₂)₂—R^{F1}. In some embodiments, R^{4A} cannot be —CH(OH)—R^{F1}. In other embodiments, R^{4A} cannot be —(CR^{G1}R^{H1})—O—R^{J1}. In some embodiments, R^{4A} cannot be —CH₂—O—R^{J1}. In still other embodiments, R^{4A} cannot be —O—(CR^{K1}R^{L1})—R^{M1}. In some embodiments, R^{4A} cannot be —O—CH₂—R^{M1}. In some embodiments, R^{4A} cannot be —(CR^{N1}R^{O1})p-R^{P1}. In some embodiments, R¹, R^{2A}, R^{3A}, R^{4B}, R^{5A} and R^{5B} cannot be each hydrogen.

In some embodiments, R^{F1} cannot be an optionally substituted bicyclic heteroaryl. In other embodiments, R^{F1} cannot be an optionally substituted bicyclic heterocyclyl. In still other embodiments, R^{F1} cannot be an optionally substituted phenyl. In some embodiments, R^{J1} cannot be an optionally substituted bicyclic heteroaryl. In other embodiments, R^{J1} cannot be an optionally substituted bicyclic heterocyclyl. In still other embodiments, R^{J1} cannot be an optionally substituted phenyl. In some embodiments, R^{M1} cannot be an optionally substituted bicyclic heteroaryl. In other embodiments, R^{M1} cannot be an optionally substituted bicyclic heterocyclyl. In still other embodiments, R^{M1} cannot be an optionally substituted phenyl. In some embodiments, R^{F1}, R^{J1} and/or R^{M1} cannot be an optionally substituted

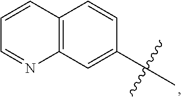 [SMILES: *c1ccc2cccnc2c1]

an optionally substituted

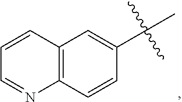 [SMILES: *c1ccc2ncccc2c1]

an optionally substituted

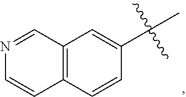 [SMILES: *c1ccc2ccncc2c1]

an optionally substituted

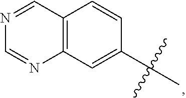 [SMILES: *c1ccc2cncnc2c1]

an optionally substituted

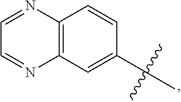 [SMILES: *c1ccc2nccnc2c1]

an optionally substituted

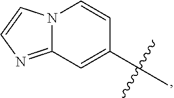 [SMILES: *c1ccn2ccnc2c1]

an optionally substituted phenyl, an optionally substituted pyridinyl (such as an optionally substituted

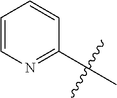 [SMILES: *c1ccccn1]

and/or an optionally substituted

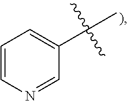 [SMILES: *c1cccnc1]

an optionally substituted

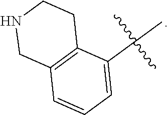 [SMILES: *c1cccc2c1CCNC2]

an optionally substituted

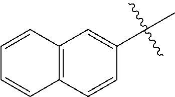 [SMILES: *c1ccc2ccccc2c1]

and/or an optionally substituted

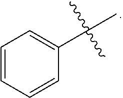 [SMILES: *c1ccccc1]

In some embodiments, R^{4B} can be an unsubstituted C₁₋₄ alkyl (such as methyl); and B¹ can be an unsubstituted or a substituted

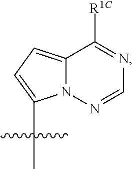

an unsubstituted or a substituted

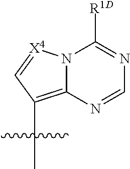

or an unsubstituted or a substituted

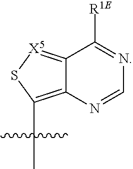

In some embodiments, R^{4B} can be an unsubstituted C₁₋₄ alkyl (such as methyl); and B¹ can be an unsubstituted or a substituted

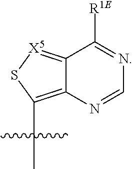

In some embodiments, R^{4B} can be an unsubstituted C₁₋₄ alkyl (such as methyl); and B¹ can be an unsubstituted or a substituted

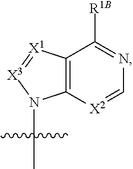

wherein X² can be CR^{C2}. In some embodiments, R^{4B} can be an unsubstituted C₁₋₄ alkyl (such as methyl); and B¹ can be an unsubstituted or a substituted

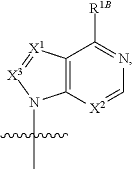

wherein X³ can be N, or an unsubstituted or a substituted

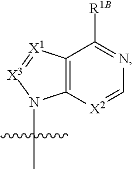

wherein X¹ can be CR^{C1}; and R^{C1} can be hydroxy, an unsubstituted C₂₋₄ alkenyl, an unsubstituted C₁₋₄ alkoxy or NR^{A1}R^{A2}.

In some embodiments, a compound of Formula (I), or a pharmaceutically acceptable salt thereof, cannot be a compound as provided in WO 2018/065354, WO 2018/154104, WO 2018/152548, WO 2018/160824, WO 2017/212385, WO 2017/032840, WO 2019/116302, WO 2020/033282, WO 2020/033285, WO 2020/033288 and/or WO 2020/205867.

### Synthesis

Compounds of Formula (I) along with those described herein may be prepared in various ways. General synthetic routes for preparing compounds of Formula (I) are shown and described herein along with some examples of starting materials used to synthesize compounds described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

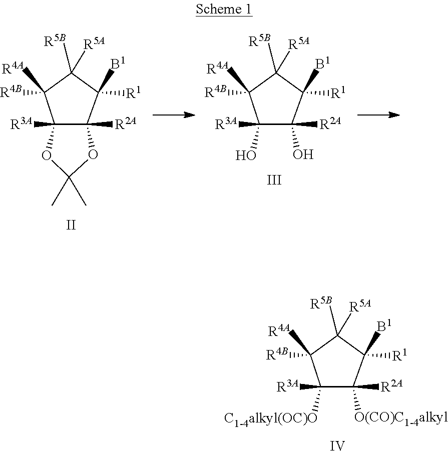

Deprotection of the acetonide of General Formula II can be performed in the presence of a suitable acid, for example HCl in MeOH, at a suitable temperature (such as room temperature), resulting in the formation of the compound of General Formula III. Optionally, compound of General Formula III can be converted to its corresponding ester of General Formula IV, by reaction with a suitable C₁₋₄ alkyl acid anhydride or C₁₋₄ alkyl acid chloride.

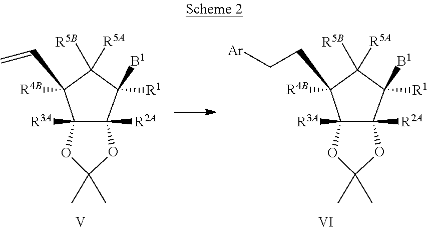

As depicted in Scheme 2, introduction of an aryl group (Ar) or in extension, if applicable, R^{F1}, on the vinyl side chain on a compound of General Formula V, is performed by a reaction of the double bond with 9-BBN under an inert atmosphere, for example in a suitable solvent under appropriate conditions. An example of a suitable solvent and temperature is THF, at 50° C. The carbon-carbon bond can then be formed with a suitable Ar—Br or Ar—I (or in extension, if applicable R^{F1}—Br or R^{F1}—I) using a suitable catalyst (such as Pd(dppf)Cl₂) in the presence of a base (for example, K₃PO₄), resulting in the formation of a compound of General Formula VI. In the context of the generic synthesis scheme, functional group conversions can be performed on the compound of General Formula VI, for example conversion of R^{4B} from —CH₂—OH via —C(═O)—H to —CN (as described in example 40), —(C═O)NH₂ (as described in example 40), vinyl (as described in example 46) or alkyne (as described in example 47).

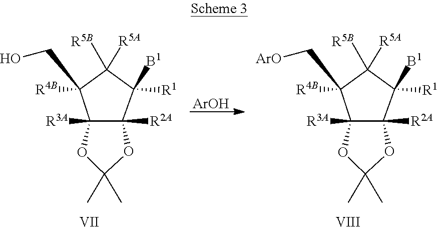

As depicted in scheme 3, Mitsunobu reaction of a compound of General Formula VII with ArOH (or in extension, if applicable, R^{J1}OH), results in the formation of a compound of General Formula VIII (where in extension ArO—, can be R^{J1}O—), for example, by using PPh₃ and DIAD in a solvent like THF, or cyanomethylenetributylphosphorane (CMBP) in a suitable solvent (for example, toluene).

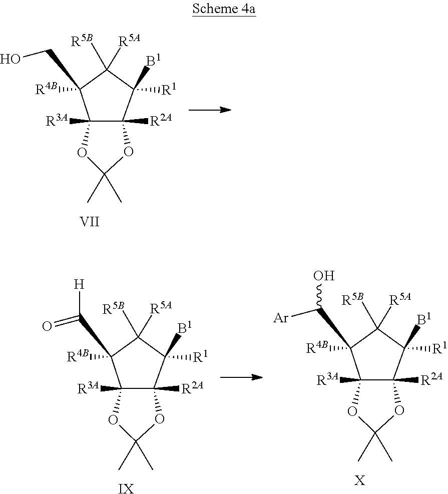

A compound of General Formula VII can be oxidized to the corresponding aldehyde of General Formula IX, followed by addition of an organometallic reagent like ArylMg(halide), or in extension, if applicable, R^{F1}—Mg(halide), resulting in the formation of a compound of General Formula X (wherein, in extension Ar— can be R^{F1}—). Alternatively, a compound of General Formula IX can be formed by oxidation of the vinyl functionality of compound of General Formula V, for example, by dihydroxylation with OsO₄, followed by oxidation with NaIO₄. A compound of General Formula V can be formed by a Wittig reaction of the aldehyde of General Formula IX.

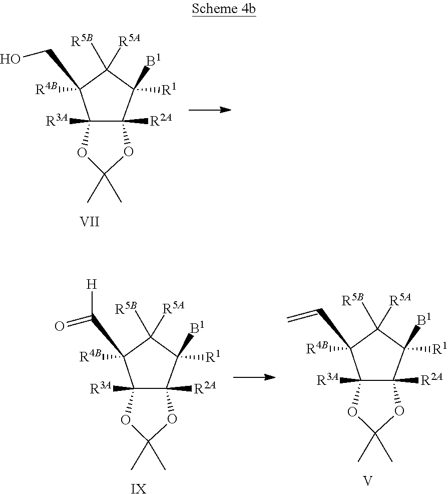

As known to those skilled in the art, the compounds of Scheme 1 to 3 can be suitably protected when required. Compounds of General Formulae VII and V can be commercially available or can be obtained by methods known to those skilled in the art. An example of a compound of General Formula VII is

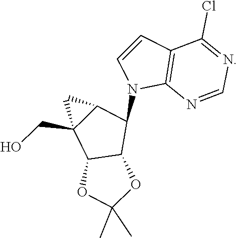

An example of a compound of General Formula V is

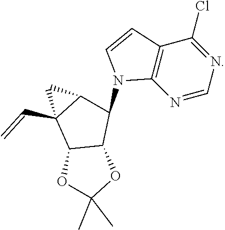

Another example of General Formulae VII and V are

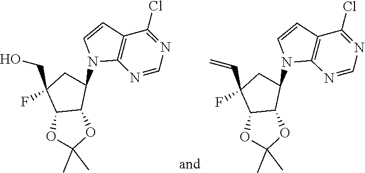

respectively, for which the synthesis is described in example 31.

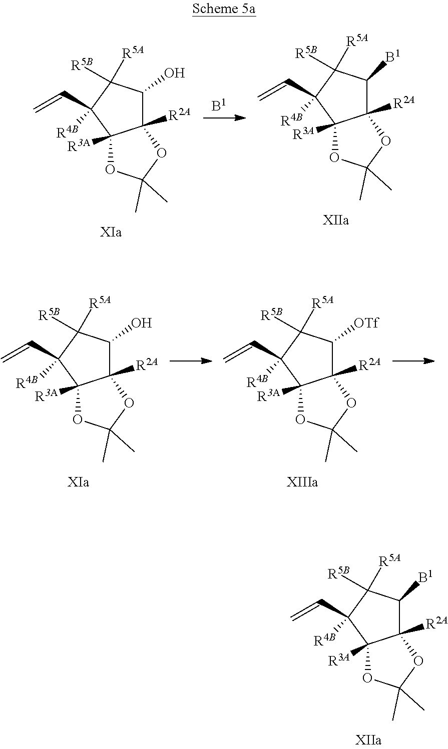

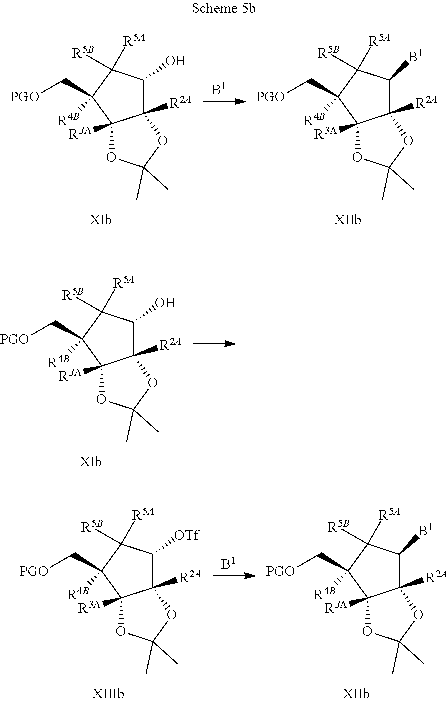

Introduction of a nucleobase described herein (denoted as B¹) can be performed as exemplified in Scheme 5a and 5b, either using Mitsunobu-like conditions, for example using DIAD and PPh₃, in THF at room temperature, and then converting the compound of General Formula XIa or XIb to a compound of General Formula XIIa or XIIb, respectively. Alternatively, General Formula XIa or XIb can be converted to a triflate of General Formula XIIIa or XIIIb, respectively. Following substitution, a compound of General Formula XIIa or XIIb, respectively, can be obtained. An example of a compound of General Formula XIB is:

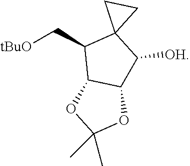

An example of a compound of General Formulae XIa and XIIa, within the context of the generic synthesis scheme, are

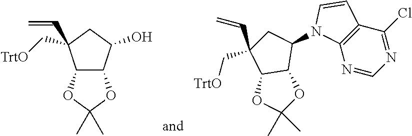

respectively, prepared as described in example 36. Trt or Trityl is a protecting group that can be removed in the course of the synthetic route. Another example of compounds of General Formulae XIa and XIIa are

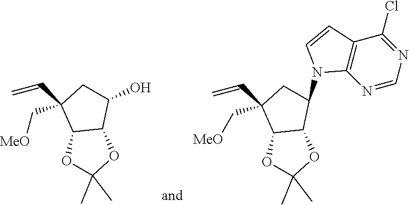

respectively, prepared as described in example 53.

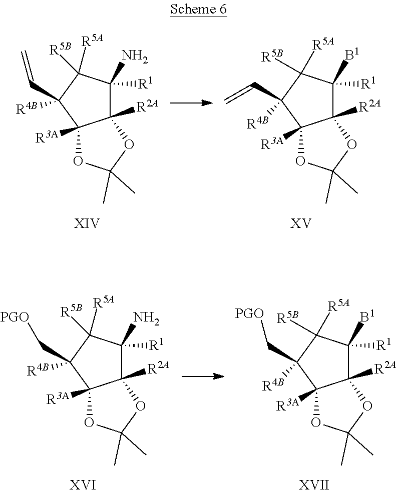

As shown in Scheme 6, when B¹ is connect to the rest of the scaffold via a nitrogen, amines of General Formulae XIV and XVI (PG represents a protection group) can be converted to compounds of General Formulae XV and XVII, respectively, using methods know to those skilled in the art. Amines of General Formulae XIV and XVI can be obtained utilizing methods known to those skilled in the art. An example of a compound of General Formula XVI is

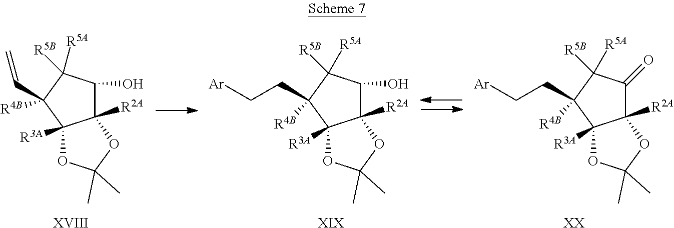

Where desired, functional group transformations can be performed on the compounds of general formulae depicted in Scheme 1 to 6, containing a B¹ group. For example, conversion of the R^{1B}, substituent from chloro to NH₂, via displacement with ammonia, such as described in example 1 for conversion of 8A to 9A. Or for example, conversion of the R^{1B}, substituent from chloro to NH₂, by palladium catalyzed coupling, such as with diphenylmethanimine, followed by removal of the protecting group as exemplified in example 17. Another example can be the conversion of R^{1B} from chloride to methyl, as exemplified in example 28.

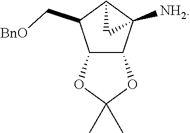 [SMILES: CC1(C)O[C@@H]2[C@@H](COCc3ccccc3)[C@@H]3C[C@]3(N)[C@@H]2O1]

As exemplified in Scheme 7, a compound of the General Formula XVIII can be converted to a compound of General Formula XIX, using methods similar to those described for the conversion of a compound of General Formula V to a compound of General Formula VI. Oxidation of the alcohol to the ketone, for example, using IBX (2-Iodoxybenzoic acid) in acetonitrile at a temperature of 60° C., can provide a compound of General Formula XX. Functional group modification on R^{5B}/R^{5A}, or introduction of R^{5B}/R^{5A} can be performed utilizing a compound of General Formula XX. For example if R^{5B} and R^{5A} are each hydrogen, introduction of an exocyclic vinyl can be performed by using an Eschenmoser's salt, followed by amine methylation under the influence of Mel and subsequent elimination. The formed ketone of General Formula XX can be, after functional group modification of R^{5B} and/or R^{5A}, reduced back to the alcohol of General Formula XIX. An example of such a ketone of General Formula XX, formed by functional group modification at the stage of a compound of General Formula XX, is:

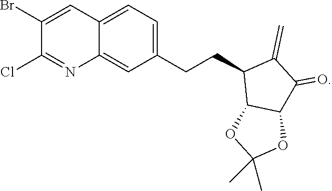

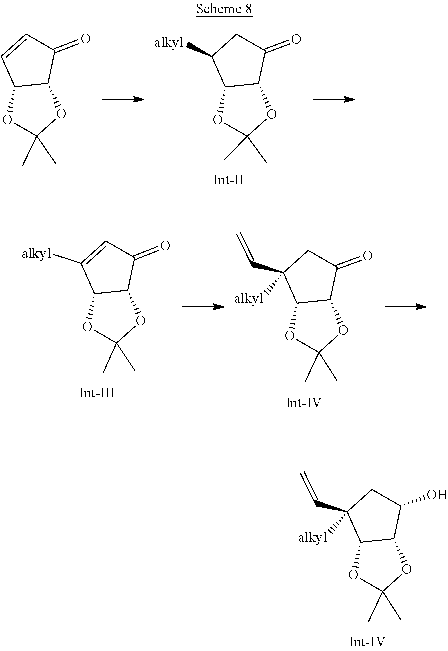

As described in Scheme 8, addition of an alkyl group as described herein to the 4′-position of the 5-membered ring of a compound of Formula (I) can be accomplished with an enone (3aR,6aR)-2,2-dimethyl-3a,6a-dihydrocyclopenta[d][1,3]dioxol-4-one. For example, using a cupper reagent, made from alkyl lithium in the presence of CuI in THF at 0° C., followed by addition to the enone at −78° C., can result in the formation of an intermediate of General Formula Int-II. Oxidation of the intermediate of General Formula Int-II to General Formula Int-III can be performed by forming the TES-enol, followed by oxidation in the presence of Pd(OAc)₂ and oxygen in DMSO at a suitable temperature (such as 60° C.). Stereoselective addition of a vinyl group to the enone of General Formula Int-III, can be performed, for example, by treating a mixture of LiCl and CuI in THF with a mixture of TMSCl and General Formula Int-III, followed by addition of vinylmagnesium bromide at 0° C. This can be followed by the deprotection of any formed silyl enolate. Treatment with an acid, like HCl, in acetone/MeOH at a suitable temperature (for example, room temperature) can provide a compound of General Formula Int-IV. The ketone can be reduced to the alcohol of General Formula Int-IV, for example, by treatment with NaBH₄ in MeOH at 0° C.

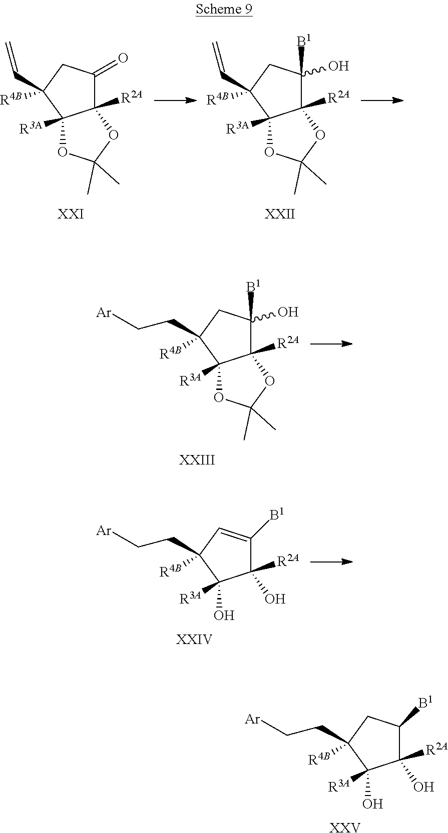

Scheme 9 describes a generic synthesis of the compounds which has B¹ connected to the five-membered ring via a carbon-carbon bond. A compound of General Formula XXII can be formed by addition of an organometallic reagent to the ketone of General Formula XXI. An example of such organometallic reagent can be generated from reacting

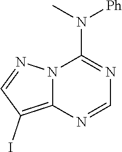

with i-PrMgCl.LiCl, as described in example 51. Another example of such organometallic reagent, can be prepared by reaction of

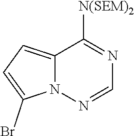

with i-PrMgCl.LiCl, as described in example 43. Further transformations involve introduction of —Ar from General Formula XXII to General Formula XXIII, similar as described for the conversion of a compound of General Formula V to a compound of General Formula VI. Elimination of the —OH of General Formula XXIII to the alkene of General Formula XXIV, can be performed under acidic conditions, or, for example, by treating with DAST. The acetonide protecting group can be removed under acidic conditions, for example, by the treatment with aqueous HCl. Reduction of the double bond in General Formula XXIV, can be accomplished by hydrogenation using a heterogeneous catalyst like PtO₂ in a suitable solvent (such as THF) under a hydrogen atmosphere. Alternatively, depending on the substituents on General Formula XXIV, Crabtree's catalyst can be used for the hydrogenation, for example, in MeOH under hydrogen atmosphere. In case diastereoisomers are obtained after the reduction, the desired isomer of General Formula XXV can be isolated out.

During the synthesis of compounds of Formula (I), such as those shown in Schemes 1-9, one or more moieties can be protected with one or more suitable protecting groups. Those skilled in the art know and can select the suitable protecting group(s) and the conditions to add and remove the suitable protecting group(s). The protecting group(s) may be chosen in such a way, that they are stable to certain reaction conditions and readily removed at a convenient stage using methodology known from the art.

### Pharmaceutical Compositions

Some embodiments described herein relate to a pharmaceutical composition, that can include an effective amount of a compound described herein (e.g., a compound, or a pharmaceutically acceptable salt thereof, as described herein) and a pharmaceutically acceptable carrier, excipient or combination thereof. A pharmaceutical composition described herein is suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Pharmaceutical compositions may be formulated in a variety forms, such as tablets, capsules or solutions for oral administration; suppositories for rectal or vaginal administration; sterile solutions or suspensions for injectable administration. Injectables can be prepared in conventional forms, either as liquid solutions or suspensions, solid forms suitable for solution or suspension in liquid prior to injection, or as emulsions.

Proper formulation is dependent upon the route of administration chosen. Techniques for formulation and administration of the compounds described herein are known to those skilled in the art. Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Pharmaceutical compositions will generally be tailored to the specific intended route of administration.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

### Methods of Use

Some embodiments described herein relate to a method of treating a cancer that can include administering to a subject identified as suffering from a cancer an effective amount of a compound described herein, or a pharmaceutically acceptable salt thereof, or a pharmaceutical composition that includes an effective amount of a compound described herein, or a pharmaceutically acceptable salt thereof. Other embodiments described herein relate to using a compound described herein, or a pharmaceutically acceptable salt thereof, in the manufacture of a medicament for treating a cancer. Still other embodiments described herein relate to the use of a compound described herein, or a pharmaceutically acceptable salt thereof, or a pharmaceutical composition that includes a compound described herein, or a pharmaceutically acceptable salt thereof, for treating a cancer. Examples of suitable cancers include lymphomas, leukemias, liver cancers, lung cancers, breast cancers, colorectal cancers and/or melanoma (such as uveal melanoma). Uveal melanoma (UM) is a rare but aggressive cancer. Most of the subject who have UM develop liver metastases and succumb within one year due to the lack of effective treatments. Compared to other tumors, UM is characterized by a high genetic stability and low mutational burden.

Some embodiments described herein relate to a method of treating a liver cancer (for example, hepatocellular carcinoma (HCC)) that can include administering to a subject identified as suffering from the liver cancer an effective amount of a compound described herein, or a pharmaceutically acceptable salt thereof, or a pharmaceutical composition that includes an effective amount of a compound described herein, or a pharmaceutically acceptable salt thereof. Other embodiments described herein relate to using a compound described herein, or a pharmaceutically acceptable salt thereof, in the manufacture of a medicament for treating a liver cancer (such as HCC). Still other embodiments described herein relate to the use of a compound described herein, or a pharmaceutically acceptable salt thereof, or a pharmaceutical composition that includes a compound described herein, or a pharmaceutically acceptable salt thereof, for treating a liver cancer (for example, HCC).

Some embodiments described herein relate to a method for inhibiting replication of a cancer cell that can include contacting the cancer cell or administering to a subject identified as suffering from HCC with an effective amount of a compound described herein, or a pharmaceutically acceptable salt thereof, or a pharmaceutical composition that includes of a compound described herein, or a pharmaceutically acceptable salt thereof. Other embodiments described herein relate to the use of an effective amount of a compound described herein, or a pharmaceutically acceptable salt thereof, or a pharmaceutical composition that includes of a compound described herein, or a pharmaceutically acceptable salt thereof, in the manufacture of a medicament for inhibiting replication of a cancer cell. Still other embodiments described herein relate to an effective amount of a compound described herein, or a pharmaceutically acceptable salt thereof, or a pharmaceutical composition that includes of a compound described herein, or a pharmaceutically acceptable salt thereof, for inhibiting replication of a cancer cell.

Some embodiments described herein relate to a method for inhibiting cell proliferation, such as inhibiting cell proliferation of cancer cells, that can include administering to a subject identified as suffering from a disease wherein inhibiting cell proliferation is desirable with an effective amount of a compound described herein, or a pharmaceutically acceptable salt thereof, or a pharmaceutical composition that includes of a compound described herein, or a pharmaceutically acceptable salt thereof. Other embodiments described herein relate to the use of an effective amount of a compound described herein, or a pharmaceutically acceptable salt thereof, or a pharmaceutical composition that includes of a compound described herein, or a pharmaceutically acceptable salt thereof, in the manufacture of a medicament for inhibiting cell proliferation, such as inhibiting cell proliferation of cancer cells. Still other embodiments described herein relate to an effective amount of a compound described herein, or a pharmaceutically acceptable salt thereof, or a pharmaceutical composition that includes of a compound described herein, or a pharmaceutically acceptable salt thereof, for inhibiting cell proliferation, such as inhibiting cell proliferation of cancer cells.

Some embodiments described herein relate to a method of modulating a PRMT5 enzyme that can include contacting a cell (for example, a cancer cell described herein) with an effective amount of a compound described herein, or a pharmaceutically acceptable salt thereof, or a pharmaceutical composition that includes an effective amount of a compound described herein, or a pharmaceutically acceptable salt thereof. Other embodiments described herein relate to using a compound described herein, or a pharmaceutically acceptable salt thereof, in the manufacture of a medicament for modulating a PRMT5 enzyme. Still other embodiments described herein relate to the use of a compound described herein, or a pharmaceutically acceptable salt thereof, or a pharmaceutical composition that includes a compound described herein, or a pharmaceutically acceptable salt thereof, for modulating a PRMT5 enzyme.

Some embodiments described herein relate to a method of inhibiting the activity of a PRMT5 enzyme that can include contacting a cell (for example, a cancer cell described herein) with an effective amount of a compound described herein, or a pharmaceutically acceptable salt thereof, or a pharmaceutical composition that includes an effective amount of a compound described herein, or a pharmaceutically acceptable salt thereof. Other embodiments described herein relate to using a compound described herein, or a pharmaceutically acceptable salt thereof, in the manufacture of a medicament for inhibiting the activity of a PRMT5 enzyme. Still other embodiments described herein relate to the use of a compound described herein, or a pharmaceutically acceptable salt thereof, or a pharmaceutical composition that includes a compound described herein, or a pharmaceutically acceptable salt thereof, for inhibiting the activity of a PRMT5 enzyme.

Some embodiments described herein relate to a method of inducing apoptosis of a cell (for example, a cancer cell described herein) that can include contacting the cell with an effective amount of a compound described herein, or a pharmaceutically acceptable salt thereof, or a pharmaceutical composition that includes an effective amount of a compound described herein, or a pharmaceutically acceptable salt thereof. Other embodiments described herein relate to using a compound described herein, or a pharmaceutically acceptable salt thereof, in the manufacture of a medicament for inducing apoptosis of a cell, such as a cancer cell described herein. Still other embodiments described herein relate to the use of a compound described herein, or a pharmaceutically acceptable salt thereof, or a pharmaceutical composition that includes a compound described herein, or a pharmaceutically acceptable salt thereof, for inducing apoptosis of a cell, such as a cancer cell described herein.

Some embodiments described herein relate to a method of decreasing the viability of a cell (for example, a cancer cell described herein) that can include contacting the cell with an effective amount of a compound described herein, or a pharmaceutically acceptable salt thereof, or a pharmaceutical composition that includes an effective amount of a compound described herein, or a pharmaceutically acceptable salt thereof. Other embodiments described herein relate to using a compound described herein, or a pharmaceutically acceptable salt thereof, in the manufacture of a medicament for decreasing the viability of a cell, such as a cancer cell described herein. Still other embodiments described herein relate to the use of a compound described herein, or a pharmaceutically acceptable salt thereof, or a pharmaceutical composition that includes a compound described herein, or a pharmaceutically acceptable salt thereof, for decreasing the viability of a cell, such as a cancer cell described herein. Exemplary cancer cells include lymphoma cells, leukemia cells, liver cancer cells, lung cancer cells, breast cancer cells and/or colorectal cancer cells. In some embodiments, the cancer cell can be a liver cancer cell.

For treatment of liver cancer, a high liver to plasma ratio can be useful. Accordingly, compounds that with a high liver to plasma ratio are of interest. In some embodiments, a compound described herein (for example, a compound of Formula (I), or a pharmaceutically acceptable salt thereof) can have a liver to plasma ratio of >5. In some embodiments, a compound described herein (for example, a compound of Formula (I), or a pharmaceutically acceptable salt thereof) can have a liver to plasma ratio of >10.

Compounds disclosed herein can be evaluated for efficacy and toxicity using known methods. A non-limiting list of potential advantages of compounds described herein (such as a compound of Formula (I), and pharmaceutically acceptable salts thereof) include improved stability, increased safety profile, increased efficacy, increased binding to the target, increased specificity for the target (for example, a cancer cell).

PRMT5 is required for the splicing of MDM4 and subsequent inactivation of WT p53 (Bezzi et al., Gene Dev. (2013) 27:1903-1916). It has been shown that WT p53 status strongly correlates with the sensitivity of cells to PRMT5 inhibitors (Gerhart et al., Sci. Rep. (2018) 8:9711). Therefore, selecting subjects that harbor a WT p53 status could represent a potential strategy to identify subjects that would benefit from the PRMT5 inhibitors. Most of the UM patients (>80%) are WT p53 and thus UM could represent a homogenous patient population that test PRMT5 inhibitors. Some embodiments disclosed herein relate to method for treating a cancer that comprises identifying a subject suffering from a cancer and possessing wild-type (WT) p53; and administering to the identified subject an effective amount of a compound described herein, such as a compound of Formula (I), and pharmaceutically acceptable salts thereof.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human.

The term “effective amount” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, including a human cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Combination Therapies

In some embodiments, a compound described herein, or a pharmaceutically acceptable salt thereof, can be used in combination with one or more additional agent(s) for treating and/or inhibiting replication HCC. Additional agents include, but are not limited to, a kinase inhibitor (such as Sorafenib, Lenvatinib and Apatinib), a checkpoint inhibitor/modulator (such as a PD1/PDL1 inhibitor, an anti-PD1 antibody, for example, Nivolumab, Keytruda® and cemiplimab, an anti-PDL1 antibody, such as atezolizumab, avelumab and durvalumab, and an anti-CTLA4 antibody, such as Tremelimumab and Ipilimumab) and an anti-VEGF antibody (such as Bevacizumab).

In some embodiments, a compound described herein, or a pharmaceutically acceptable salt thereof, can be administered with one or more additional agent(s) together in a single pharmaceutical composition. In some embodiments, a compound described herein, or a pharmaceutically acceptable salt thereof, can be administered with one or more additional agent(s) as two or more separate pharmaceutical compositions. Further, the order of administration of a compound described herein, or a pharmaceutically acceptable salt thereof, with one or more additional agent(s) can vary.

### EXAMPLES

Table of Abbreviations:
The following abbreviations may appear in the present disclosure:

| Abbreviation | Name |
|---|---|
| Ac | Acetate |
| ACN | Acetonitrile |
| anhyd. | Anhydrous |
| aq. | Aqueous |
| BPO | Benzoylperoxide |
| Bu | Butyl |
| CAN | Ceric ammonium nitrate |
| conc. | Concentrated |
| DCM | Dichloromethane |
| DIPEA | N,N-Diisopropylethylamine |
| DMA | N,N-dimethylacetamide |
| DMF | N,N-Dimethylformamide |
| DMSO | Dimethyl sulfoxide |
| DPPA | Diphenylphosphoryl azide |
| Dppf | 1,1′-Bis(diphenylphosphino)ferrocene |
| EA═EtOAc | Ethyl acetate |
| ECF | Ethyl chloroformate |
| Et | Ethyl |
| FA | Formic acid |
| g | Gram(s) |
| h | Hour(s) |
| IBX | 2-Iodoxybenzoic acid |
| Me | Methyl |
| MeOH | Methanol |
| Min | Minute(s) |
| NBS | N-Bromosuccinimide |
| NIS | N-Iodosuccinimide |
| PE | Petroleum ether |
| rt | Room temperature |
| sat. | Saturated |
| TBAF | Tetra-n-butylammonium fluoride |
| TBSCL | t-Butyldimethylsilyl chloride |
| TFA | Trifluoroacetic acid |
| THF | Tetrahydrofuran |

Additional embodiments are disclosed in further detail in the following examples, which are not in any way intended to limit the scope of the claims.

### Example 1

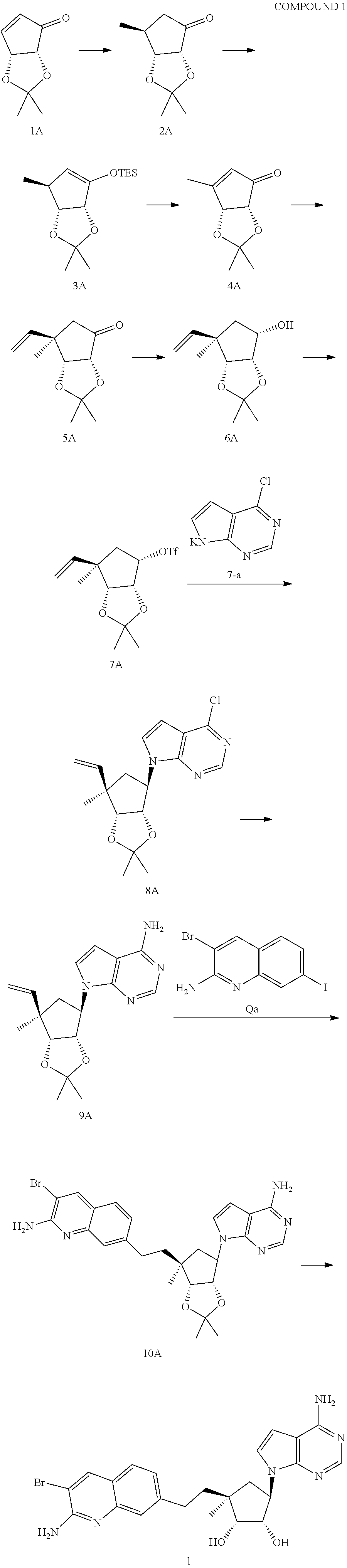

MeLi (1.6 M in Et₂O, 57.50 mL, 2.3 eq.) was added dropwise to a mixture of CuI (9.14 g, 48.00 mmol, 1.2 eq.) in THF (10 mL) at 0° C. The mixture was stirred at 0° C. for 10 min, then a solution of (3aR,6aR)-2,2-dimethyl-3a,6a-dihydrocyclopenta[d][1,3]dioxol-4-one (1A) (6.17 g, 40 mmol, 1 eq.) in THF (10 mL) was added dropwise at −78° C. The mixture was stirred at −78° C. for 20 min. The reaction progress was monitored by TLC (PE:EA=5:1). Upon completion, the reaction was quenched by NH₄Cl (sat. aq, 50 mL) and extracted with EA (2×50 mL). The combined organic layers were washed with brine (20 mL), dried over Na₂SO₄, and concentrated to give a residue. The residue was purified by silica gel chromatography (Petroleum ether:Ethyl acetate (PE:EA)=10:1) to afford 3aR,6S,6aR)-2,2,6-trimethyl-3a,5,6,6a-tetrahydrocyclopenta[d][1,3]dioxol-4-one (2A) (5.2 g, 30.55 mmol, 76% yield) as a colorless oil.

LiHMDS (1.0 M in THF, 49.35 mL, 1.5 eq.) was added dropwise to a mixture of 2A (5.6 g, 32.90 mmol, 1 eq.) and chlorotriethylsilane (9.92 g, 65.80 mmol, 11.19 mL, 2 eq.) in THF (60 mL) at −78° C. The mixture was stirred at −78° C. for 10 min. The reaction progress was monitored by TLC (PE:EA=10:1). Upon completion, the mixture was quenched by NH₄Cl (sat. aq., 80 mL) and brine (50 mL), and extracted with MTBE (2×100 mL). The combined organic layers were washed with brine (80 mL), dried over Na₂SO₄, and concentrated to give a residue. The residue was purified by silica gel chromatography (PE:EA=50:1) to afford [(3aR,6aR)-2,2,6-trimethyl-6,6a-dihydro-3aH-cyclopenta[d][1,3]dioxol-4-yl]oxy-triethyl-silane (3A) (7.9 g, 27.77 mmol, 84% yield) as a colorless oil.

A mixture of 3A (7.9 g, 27.77 mmol, 1 eq.) and Pd(OAc)₂ (1.87 g, 8.33 mmol, 0.3 eq.) in DMSO (80 mL) was stirred at 60° C. under O₂ atmosphere for 6 h. The reaction progress was monitored by TLC (PE:EA=10:1). Upon completion, the mixture was diluted with water (300 mL) and extracted with MBTE (2×100 mL). The combined organic layers were combined and washed with water (100 mL) and brine (80 mL), dried over anhydrous Na₂SO₄, and concentrated to give a residue. The residue was purified by silica gel chromatography (PE:EA=5:1) to afford (3aR,6aR)-2,2,6-trimethyl-3a,6a-dihydrocyclopenta[d][1,3]dioxol-4-one (4A) (3.7 g, 22.00 mmol, 79% yield) as a colorless oil.

A mixture of LiCl (8.48 mg, 200.00 μmol, 4.10 μL, 0.2 eq.), CuI (19.04 mg, 100.00 μmol, 0.1 eq.) in THF (2 mL) was stirred at 0° C. for 10 min. Then a mixture of TMSCl (130.37 mg, 1.20 mmol, 152.30 μL, 1.2 eq.) and 4A (168.19 mg, 1 mmol, 1 eq.) in THF (1 mL) was added dropwise at 0° C., and string was continued at 0° C. for 20 min. Vinylmagnesium bromide (1 M in THF, 1.60 mL, 1.6 eq.) was added dropwise at 0° C., and the mixture was stirred at 0° C. for 30 min. The reaction progress was monitored by TLC (PE:EA=5:1). Upon completion, the reaction was quenched by NH₄Cl (sat. aq., 10 mL) and extracted with EA (2×20 mL). The combined organic layers were washed with brine (20 mL), dried over anhydrous Na₂SO₄, and concentrated to give a mixture of 5A and silyl enolate. The mixture was dissolved in acetone (2 mL) and MeOH (2 mL), cone. HCl (0.05 mL) was added, and the mixture was stirred at rt for 10 min. TEA (1 mL) was added to the mixture to quench the reaction. The solvent was removed under reduced pressure to give a residue. The residue was purified by silica gel chromatography (PE/EA=10:1) to afford (3aR,4R,6aR)-2,2,4-trimethyl-4-vinyl-5,6a-dihydro-3aH-cyclopenta[d][1,3]dioxol-6-one (5A) (97 mg, 494.29 μmol, 49% yield) as a colorless oil.

NaBH₄ (35.5 mg, 937.62 μmol, 2 eq.) was added to a mixture of 5A (92 mg, 468.81 μmol, 1 eq.) and THF (1 mL) at 0° C. The mixture was stirred at 0° C. for 0.5 h. The reaction progress was monitored by TLC (PE:EA=10:1). Upon completion, the mixture was quenched by acetone (0.5 mL) and then concentrated to give a residue. The residue was diluted with aqueous potassium sodium tartrate (sat. aq., 20 mL) and then extracted with EA (2×20 mL). The combined organic layers were washed with brine (10 mL), dried over anhydrous Na₂SO₄, and concentrated to give a residue. The residue was purified by silica gel chromatography (PE:EA=20:1) to afford (3aR,4R,6S,6aS)-2,2,4-trimethyl-4-vinyl-3a,5,6,6a-tetrahydrocyclopenta[d][1,3]dioxol-6-ol (6A) (67 mg, 337.54 μmol, 72% yield) as a colorless oil.

Trifluoromethanesulfonic anhydride (423.21 mg, 1.50 mmol, 247.49 μL, 1.5 eq.) was added to a mixture of 6A (198.26 mg, 1 mmol, 1 eq.) and pyridine (316.40 mg, 4.00 mmol, 322.86 μL, 4 eq.) in DCM (5 mL) at 0° C. The mixture was stirred at 0° C. for 1 h. The reaction progress was monitored by TLC (PE:EA=10:1). The mixture was quenched by ice-water (10 mL) and extracted with DCM (2×15 mL). The combined organic layers were washed with brine (10 mL), dried over anhydrous Na₂SO₄, and concentrated to give crude (7A) (330 mg) as a yellow oil, which was used for next step without further purification.

A solution of (4-chloropyrrolo[2,3-d]pyrimidin-7-yl)potassium (7-a) (191.47 mg, 999.03 μmol, 1 eq.) in DMF (1 mL) was added dropwise to a solution of crude 7A (330 mg) in DMF (3 mL) at 0° C. The mixture was stirred at rt for 36 h. The reaction progress was monitored by TLC (PE:EA=5:1). The mixture was diluted with water (20 mL) and extracted with EA (2×20 mL). The combined organic layers were washed with water (20 mL) and brine (30 mL), dried over anhydrous Na₂SO₄, and concentrated to give a residue. The residue was purified by silica gel chromatography (PE:EA=10:1) to afford 7-[(3aR,4R,6R,6aS)-2,2,4-trimethyl-4-vinyl-3a,5,6,6a-tetrahydrocyclopenta[d][1,3]dioxol-6-yl]-4-chloro-pyrrolo[2,3-d]pyrimidine (8A) (134 mg, 397.41 μmol, 40% yield) as a colorless gum.

A mixture of 8A (700 mg, 2.10 mmol, 1 eq.) and NH₃.H₂O (28.00 g, 199.74 mmol, 30.77 mL, 95.25 eq.) in dioxane (16 mL) was stirred at 100° C. for 60 h. The reaction progress was monitored by LCMS. Upon completion, the mixture was concentrated under reduced pressure to give a residue. The residue was purified by silica gel chromatography (PE:EA=6:1, 200 mL, DCM:MeOH=20:1, 200 mL) to give 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (490 mg, 1.56 mmol, 74% yield) as a yellow foam.

To a solution of 9A (314.38 mg, 1 mmol, 1 eq.) in THF (5 mL) was added 9-BBN dimer (532.44 mg, 2.20 mmol, 2.2 eq.). The mixture was stirred at 50° C. for 2 h and then cooled to 20° C. K₃PO₄ (1.06 g, 5.00 mmol, 5 eq.). To the mixture was added H₂O (0.5 mL), and the mixture was stirred at rt for 0.5 h. 3-bromo-7-iodo-quinolin-2-amine (Qa) (488.55 mg, 1.40 mmol, 1.4 eq.) and Pd(dppf)Cl₂ (73.17 mg, 100.00 μmol, 0.1 eq.) were added to the mixture. The mixture was stirred at 60° C. for 12 h. The reaction progress was monitored by LCMS. Upon completion, the mixture was diluted with brine (20 mL) and extracted with EA (3×30 mL). The combined organic layers were dried over anhydrous Na₂SO₄ and concentrated to give a residue. The residue was purified by silica gel chromatography (EA=100%, 200 mL; DCM:MeOH=20:1, 500 mL) to afford the crude product, which was further purified by prep-HPLC (column: Phenomenex Gemini-NX 150*30 mm*5 um; mobile phase: [water (0.04% NH₃H₂O+10 mM NH₄HCO₃)-ACN]; B %: 42%-72%, 8 min) to afford 7-[(E)-2-[(3aR,4R,6R,6aS)-6-(4-aminopyrrolo[2,3-d]pyrimidin-7-yl)-2,2,4-trimethyl-3a,5,6,6a-tetrahydrocyclopenta[d][1,3]dioxol-4-yl]vinyl]-3-bromo-quinolin-2-amine (10A) (350 mg, 653.67 μmol, 65% yield) as a yellow foam.

A mixture of 10A (150 mg, 280.15 μmol, 1 eq.) in MeOH (15 mL) and HCl (4 M, 4 mL, 57.11 eq.) was stirred at rt for 12 h. The reaction progress was monitored by LCMS. Upon completion, the mixture was concentrated under reduced pressure to afford a residue. The residue was suspended in MeOH (4 mL) and neutralized by NH₄OH (aq., 25%) to reach pH 8.0. The solid was dissolved first and then precipitated again. The resulting suspension was filtered and the collected solid was washed with water to afford (1S,2R,3R,5R)-3-[(E)-2-(2-amino-3-bromo-7-quinolyl)vinyl]-5-(4-aminopyrrolo[2,3-d]pyrimidin-7-yl)-3-methyl-cyclopentane-1,2-diol (1) (130 mg, 262.43 μmol, 94% yield) as an off-white solid. LCMS: (ESI): m/z calcd. for C₂₃H₂₆BrN₆O₂ 497.13 [M+H]⁺, found 497.1. ¹H NMR (400 MHz, DMSO-d₆) δ: 8.32 (s, 1H), 8.03 (s, 1H), 7.58 (d, J=8.2 Hz, 1H), 7.33 (s, 1H), 7.27 (d, J=3.5 Hz, 1H), 7.12 (br d, J=8.4 Hz, 1H), 6.91 (br s, 2H), 6.56 (br s, 2H), 6.53 (d, J=3.3 Hz, 1H), 4.94-4.83 (m, 2H), 4.60 (d, J=5.5 Hz, 1H), 4.39 (q, J=6.4 Hz, 1H), 3.77 (t, J=5.7 Hz, 1H), 2.83-2.59 (m, 2H), 1.96-1.66 (m, 4H), 1.10 (s, 3H).

### Example 2

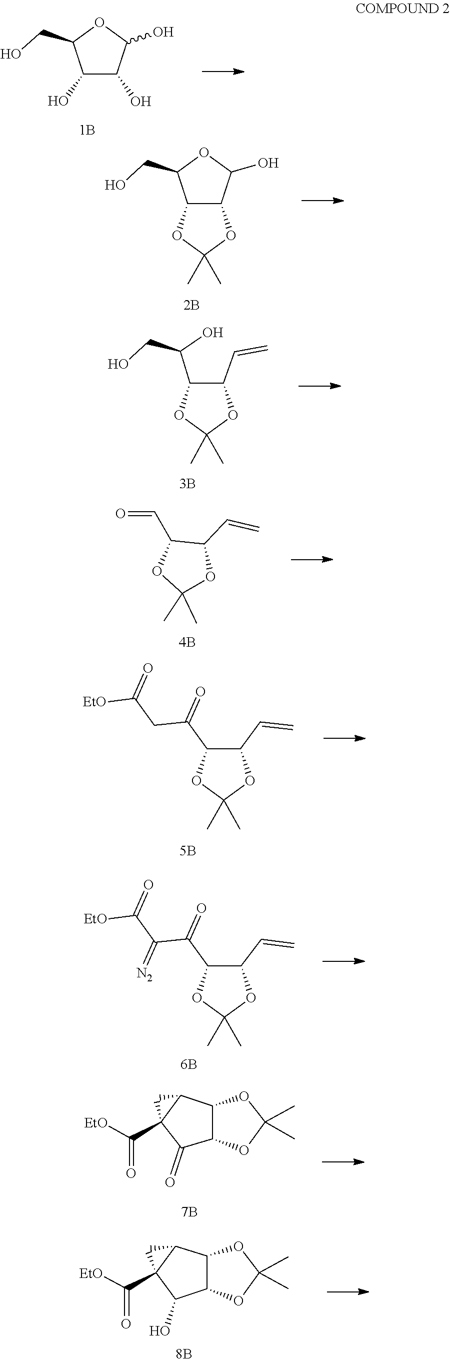

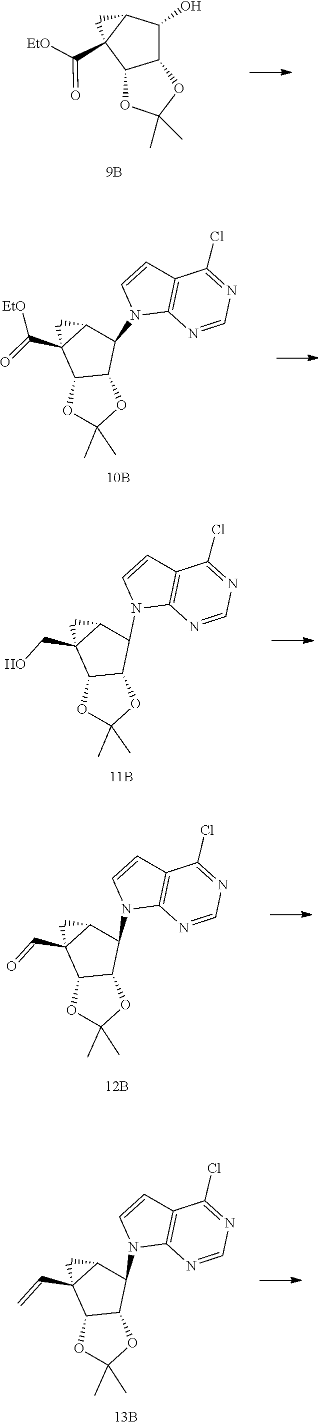 [SMILES: CCOC(=O)[C@@]12C[C@@H]1[C@H](O)[C@@H]1OC(C)(C)O[C@@H]12 ; CCOC(=O)[C@@]12C[C@@H]1[C@@H](n1ccc3c(Cl)ncnc31)[C@@H]1OC(C)(C)O[C@@H]12 ; CC1(C)O[C@H]2C(n3ccc4c(Cl)ncnc43)[C@H]3C[C@@]3(CO)[C@H]2O1 ; CC1(C)O[C@H]2[C@H](n3ccc4c(Cl)ncnc43)[C@H]3C[C@@]3(C=O)[C@H]2O1 ; C=C[C@@]12C[C@@H]1[C@@H](n1ccc3c(Cl)ncnc31)[C@@H]1OC(C)(C)O[C@@H]12]

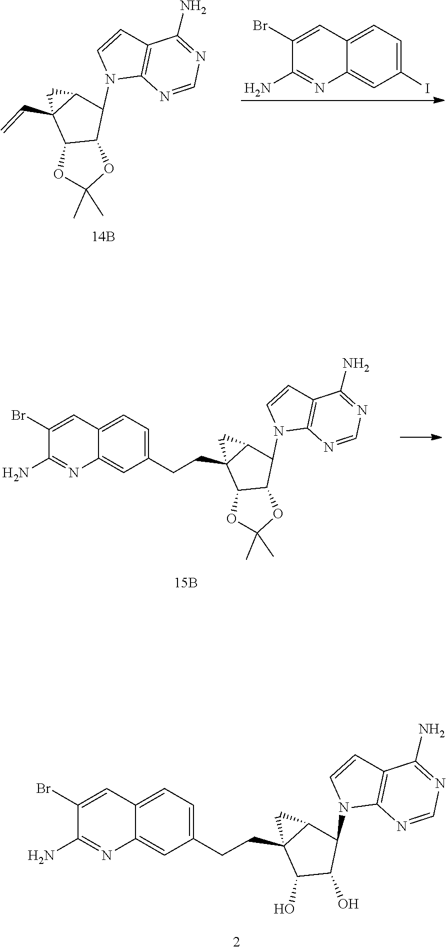 [SMILES: C=C[C@@]12C[C@@H]1C(n1ccc3c(N)ncnc31)[C@@H]1OC(C)(C)O[C@@H]12 ; Nc1nc2cc(I)ccc2cc1Br ; CC1(C)O[C@H]2C(n3ccc4c(N)ncnc43)[C@H]3C[C@@]3(CCc3ccc4cc(Br)c(N)nc4c3)[C@H]2O1 ; Nc1nc2cc(CC[C@@]34C[C@@H]3[C@@H](n3ccc5c(N)ncnc53)[C@H](O)[C@@H]4O)ccc2cc1Br]

To a solution of 1B (50 g, 333.04 mmol, 1 eq.) in acetone (500 mL) was added 2,2-dimethoxypropane (36.42 g, 349.70 mmol, 42.85 mL, 1.05 eq.) and TsOH.H₂O (633.51 mg, 3.33 mmol, 0.01 eq.) in one portion at 25° C. under N₂. The mixture was stirred at 25° C. for 12 h. The mixture was neutralized by solid NaHCO₃ to reach PH 8.0 and then filtrated. The filtrate was concentrated to give a residue as brown oil. The residue was purified by column chromatography (SiO₂, PE:EA=5:1 to 1:1) to give 2B (55 g, 274.72 mmol, 82.49% yield, 95% purity) as a brown oil.

To a solution of Ph₃PMeBr (88.32 g, 247.25 mmol, 2.85 eq.) in THF (800 mL) was added t-BuOK (32.79 g, 277.61 mmol, 95% purity, 3.2 eq.) at 0° C. The mixture was stirred at 0° C. for 0.5 h and then at 20° C. for 1 h. A solution of 2B in THF (200 mL) was added dropwise to the mixture at 0° C. during a period of 0.5 h, and the mixture was stirred at 20° C. for an additional 12 h. Upon completion, the mixture was diluted with H₂O (400 mL) and EA (500 mL). The mixture was extracted with EA (2×200 mL). The combined organic phase was washed with brine (100 mL), dried over anhydrous Na₂SO₄, filtered and concentrated in vacuum to give a residue. The residue was purified by column chromatography (SiO₂, PE:EA=5:1 to 1:1) to give 3B (25 g, crude) as a brown oil.

To a mixture of 3B in DCM (30 mL) was added a solution of NaIO₄ (3.41 g, 15.94 mmol, 883.20 μL, 1 eq.) in H₂O (20 mL) at 25° C., and the mixture was stirred at 25° C. for 1 h. Upon completion, the mixture was diluted with DCM (60 mL) and water (50 mL). The aqueous phase was extracted with DCM (2×20 mL). The combined organic phase was washed with brine (50 mL), dried over anhydrous Na₂SO₄, filtered and concentrated in vacuum. The residue was purified by column chromatography (SiO₂, PE:EA=15:1 to 5:1) to give 4B (2 g, 12.81 mmol, 80.34% yield) as a brown oil.

To a mixture of 4B (1.2 g, 7.68 mmol, 1 eq.) in anhydrous DCM (8 mL) was added SnCl₂ (174.83 mg, 922.02 μmol, 23.92 μL, 0.12 eq.) and a solution of ethyl 2-azidoacetate (1.09 g, 8.45 mmol, 1.19 mL, 1.1 eq.) in DCM (5 mL) at 0° C. The reaction was stirred at 25° C. for 1 h and then filtrated through a pad of Celite. The filtrate was concentrated to give a residue as a brown oil. The residue was purified by column chromatography (SiO₂, PE:EA=50:1 to 15:1) to give 5B (1.2 g, 4.46 mmol, 58.02% yield, 90% purity) as a brown oil.

To a solution of 5B (1.2 g, 4.95 mmol, 1 eq.) and N-diazo-4-methyl-benzenesulfonamide (976.84 mg, 4.95 mmol, 1 eq.) in CH₃CN (10 mL) was added TEA (LOO g, 9.91 mmol, 1.38 mL, 2 eq.) dropwise at 0° C. The reaction was stirred at 0° C. for 0.5 h and then at 25° C. for another 1.5 h. The mixture was diluted with EA (20 mL) and water (10 mL). The organic layer was separated and concentrated under reduce pressure to give a residue as a brown oil. The residue was purified by column chromatography (SiO₂, PE:EA=50:1 to 20:1) to give 6B (1 g, 3.35 mmol, 67.73% yield, 90% purity) as a brown oil.

To a solution of 6B (400.00 mg, 1.49 mmol, 1 eq.) in toluene (4 mL) was added CuI (14.20 mg, 74.55 μmol, 0.05 eq.) at 25° C. The reaction was stirred at 110° C. for 12 h and then diluted with EA (60 mL) and water (40 mL). The aqueous phase was extracted with EA (2×20 mL). The combined organic phase was washed with brine (15 mL), dried over anhydrous Na₂SO₄, filtered and concentrated in vacuum. The residue was purified by column chromatography (SiO₂, PE:EA=60:1 to 10:1) to give 7B (200 mg, 832.46 μmol, 27.92% yield) as a brown oil.

To a solution of 7B (0.22 g, 915.71 μmol, 1 eq.) in MeOH (6 mL) was added NaBH₄ (34.64 mg, 915.71 μmol, 1 eq.) at 25° C. The reaction was stirred at 25° C. for 1 h. Acetone (1 mL) was added, and the mixture was stirred for 5 min. The reaction was concentrated in vacuum to give a residue. The residue was purified by column chromatography (SiO₂, PE:EA=8:1 to 2:1) to give 8B (200 mg, 825.53 μmol, 90.15% yield) as a brown oil.

To a solution of 8B (0.2 g, 832.46 μmol, 1 eq.) in acetone (8 mL) was added TsOH.H₂O (79.17 mg, 416.23 μmol, 0.5 eq.) at 25° C. The reaction was stirred at 80° C. for 8 h. TEA (0.5 mL) was added to the mixture. The mixture was diluted with EA (60 mL) and water (40 mL). The aqueous phase was extracted with EA (2×20 mL). The combined organic phase was washed with brine (15 mL), dried over anhydrous Na₂SO₄, filtered and concentrated in vacuum. The residue was purified by column chromatography (SiO₂, PE:EA=6:1 to 2:1) to give 9B (160 mg, crude) as a brown oil.

To a solution of triphenyl phosphine (2.60 g, 9.91 mmol, 2 eq.) and 4-chloro-7H-pyrrolo[2,3-d]pyrimidine (1.52 g, 9.91 mmol, 2 eq.) in THF (20 mL) was added diisopropylazodicarboxylate (2.00 g, 9.91 mmol, 1.93 mL, 2 eq.) at 20° C. The mixture was stirred at 20° C. for 0.5 h. The mixture was added to a solution of 9B (1.2 g, 4.95 mmol, 1 eq.) in THF (20 mL) dropwise at 20° C. The reaction was stirred at 20° C. for 2 h, and then concentrated in vacuum to give a residue. The residue was purified by column chromatography (SiO₂, PE:EA=8:1 to 2:1) to give 10A (1.5 g, crude), which was then purified by prep-HPLC (Neutral condition) (column: Xtimate C18 150*25 mm*5 um; mobile phase: [water (10 mM NH₄HCO₃)-ACN]; B %: 63%-63%, 10 min) to give 10A (0.8 g, 2.08 mmol, 41.89% yield, 98% purity) as a brown solid.

To a solution of 10A (0.93 g, 2.46 mmol, 1 eq.) in DCM (20 mL) was added DIBAL-H (1 M in toluene, 6.15 mL, 2.5 eq.) at −78° C. The reaction was stirred at −78° C. for 1.5 h. The reaction was treated sequentially with H₂O (1 mL) and NaOH (aq. 2M, 1 mL) and stirred for 5 min. Additional H₂O (1 mL) was added, and the mixture was stirred for another 5 min. The mixture was filtrated through a pad of Celite and concentrated in vacuum to give 11A (830 mg, crude) as a brown oil, which was used for the next step without further purification.

To a solution of 11B (100 mg, 297.81 μmol, 1 eq.) in CH₃CN (3 mL) was added IBX (125.09 mg, 446.71 μmol, 1.5 eq.) at 25° C. The reaction was stirred at 80° C. for 1.5 h and then concentrated in vacuum to give 12B (100 mg, crude) as a brown oil. The residue was used for the next step without further purification.

To a solution of Ph₃PMeBr (267.57 mg, 749.02 μmol, 2.5 eq.) in THF (2 mL) was added t-BuOK (84.05 mg, 749.02 μmol, 2.5 eq.) at 0° C. The reaction was stirred at 0° C. for 10 min, and then at 20° C. for 20 min. A solution of 12B (100 mg, 299.61 μmol, 1 eq.) in THF (1.5 mL) was added dropwise at 0° C., and the reaction was stirred at 20° C. for another 0.5 h. Upon completion, the reaction was diluted with H₂O (20 mL) and EA (50 mL). The aqueous phase was extracted with EA (60 mL). The combined organic phase was washed with brine (10 mL), dried over anhydrous Na₂SO₄, filtered and concentrated in vacuum. The residue was purified by column chromatography (SiO₂, PE:EA=12:1 to 5:1) to give 13B (90 mg, 271.25 μmol, 90.54% yield, 100% purity) as a brown oil.

A mixture of 13B (220 mg, 663.06 μmol, 1 eq.) and NH₃.H₂O (24.38 g, 194.75 mmol, 26.79 mL, 28% purity, 293.71 eq.) in dioxane (10 mL) was stirred at 100° C. for 72 h in a steel sealed tube. The mixture was concentrated in vacuum to give a residue as a brown oil. The residue was purified by column chromatography (SiO₂, DCM:MeOH=200:1 to 100:2) to give 13B (350 mg, 1.09 mmol, 81.96% yield, 97% purity, from parallel 2 batches) as a brown oil.

To a solution of 14B (350 mg, 1.12 mmol, 1 eq.) in THF (12 mL) was added 9-BBN (solid, dimer, 542.35 mg, 2.24 mmol, 2 eq.) at 20° C. The mixture was degassed and then stirred at 50° C. for 2 h. The reaction was cooled to 20° C. and treated with a solution of K₃PO₄ (1.19 g, 5.60 mmol, 5 eq.) in H₂O (1.2 mL). Stirring was continued for another 30 min. To the mixture were added 3-bromo-7-iodo-quinolin-2-amine (586.51 mg, 1.68 mmol, 1.5 eq.) and Pd(dppf)Cl₂ (81.99 mg, 112.05 μmol, 0.1 eq.). The mixture was degassed, stirred at 60° C. for another 15 h, and diluted with EA (50 mL) and water (30 mL). The aqueous phase was extracted with EA (50 mL). The combined organic phase was washed with brine (30 mL), dried over anhydrous Na₂SO₄, filtered and concentrated in vacuum to give a residue as a brown oil. The residue was purified by column chromatography (SiO₂, DCM:MeOH=200:1 to 20:1) to give 15B (360 mg, 611.84 μmol, 54.61% yield, 91% purity) as a brown solid. The product was then purified by reversed-phase HPLC (Neutral condition) (column: Xtimate C18 150*25 mm*5 um; mobile phase: [water (10 mM NH₄HCO₃)-ACN]; B %: 46%-76%, 11 min) to give 15B (350 mg, 647.14 μmol, 80.58% yield, 99% purity) as a white solid.

To a solution of 15B (350 mg, 653.67 μmol, 1 eq.) in THF (10 mL) was added HCl (aq., 4 M, 5 mL, 30.60 eq.) at 20° C. The reaction was stirred at 20° C. for 5 h and then concentrated in vacuum to give crude product (315 mg, HCl salt) as a brown oil. The crude product (280 mg) was purified by reversed-phase HPLC (column: Agela DuraShell 150 mm_25 mm_5 um; mobile phase: [water (0.05% HCl)-ACN]; B %: 0%-30%, 8 min) to give 2 (160 mg, 322.99 μmol, 61.35% yield) as a white solid. MS: (ESI): m/z calcd. for C₂₃H₂₄BrN₆O₂ 495.11 [M+H]⁺, found 495.3. ¹H NMR (400 MHz, CD₃OD) δ: 8.79 (s, 1H), 8.27 (s, 1H), 7.81 (d, J=8.3 Hz, 1H), 7.65 (s, 1H), 7.51 (dd, J=1.4, 8.2 Hz, 1H), 7.38 (d, J=3.8 Hz, 1H), 6.94 (d, J=3.8 Hz, 1H), 5.05 (s, 1H), 4.67 (d, J=6.8 Hz, 1H), 3.96 (d, J=6.8 Hz, 1H), 3.20-2.96 (m, 2H), 2.41-2.26 (m, 1H), 1.88 (ddd, J=6.3, 11.0, 13.9 Hz, 1H), 1.46-1.33 (m, 2H), 0.74-0.55 (m, 1H).

### Example 2

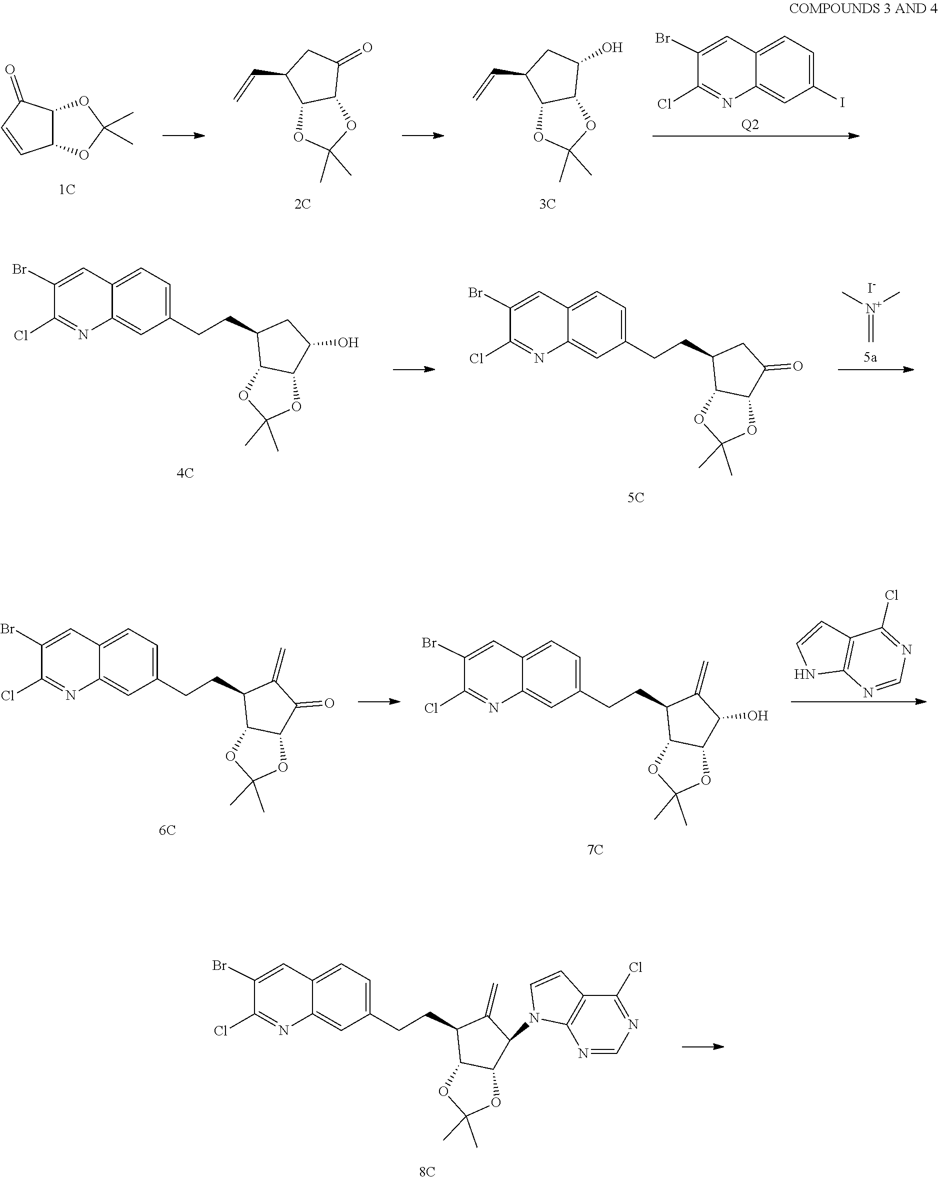

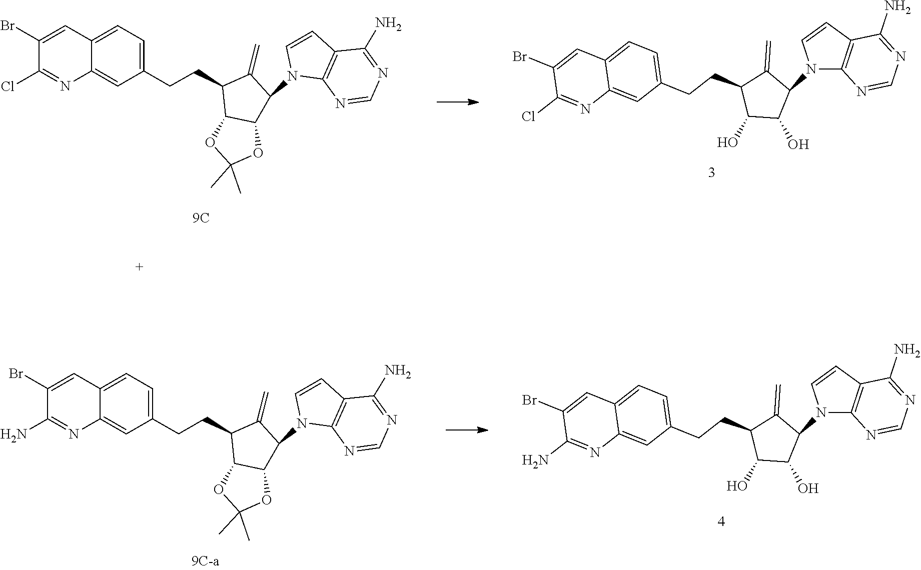 [SMILES: C=C1[C@@H](n2ccc3c(N)ncnc32)[C@@H]2OC(C)(C)O[C@@H]2[C@H]1CCc1ccc2cc(Br)c(Cl)nc2c1 ; C=C1[C@@H](n2ccc3c(N)ncnc32)[C@H](O)[C@H](O)[C@H]1CCc1ccc2cc(Br)c(Cl)nc2c1 ; C=C1[C@@H](n2ccc3c(N)ncnc32)[C@@H]2OC(C)(C)O[C@@H]2[C@H]1CCc1ccc2cc(Br)c(N)nc2c1 ; C=C1[C@@H](n2ccc3c(N)ncnc32)[C@H](O)[C@H](O)[C@H]1CCc1ccc2cc(Br)c(N)nc2c1]

TMEDA (5.65 g, 48.65 mmol, 7.34 mL, 1.5 eq.) was added to a mixture of CuI (339.73 mg, 1.78 mmol, 0.055 eq.) in THF (125 mL) at 0° C. The mixture was stirred at 0° C. for 5 min, and then cooled to −78° C. A solution of vinylmagnesium bromide (1 M in THF, 48.65 mL, 1.5 eq.) was added, and the mixture was stirred at −78° C. for 20 min. TMSCl (4.23 g, 38.92 mmol, 4.94 mL, 1.2 eq.) was added, followed by a solution of 1C (5 g, 32.43 mmol, 1 eq.) in THF (35 mL). The mixture was stirred at −78° C. for 3 h. The reaction progress was monitored by TLC (PE:EA=5:1). Upon completion, the reaction was quenched by NH₄Cl (sat., aq., 50 mL), and the mixture was extracted with EA (2×100 mL). The combined organic layers were washed with brine (2×100 mL), dried over Na₂SO₄, filtered and concentrated to give a residue. The residue was purified by column chromatography (SiO₂, PE:EA=20:1 to 10:1) to afford 2C (3.8 g, 18.77 mmol, 57% yield) as a yellow oil.

NaBH₄ (151.3 mg, 4.00 mmol, 2 eq.) was added to a mixture of 2C (364.4 mg, 2 mmol, 1 eq.) in MeOH (30 mL) at 0° C. The mixture was stirred at 0° C. for 0.5 h. The reaction progress was monitored by TLC (PE:EA=5:1). Upon completion, the mixture was quenched by acetone (5 mL) and then concentrated under reduced pressure to afford a residue. The residue was diluted with brine (50 mL) and then extracted with EA (2×50 mL). The combined organic layers were dried over anhydrous Na₂SO₄, filtered and concentrated to afford a crude product. The crude product was purified by silica gel chromatography (PE:EA=20:1) to afford 3C (260.0 mg, 1.41 mmol, 71% yield) as a colorless oil.

9-BBN dimer (181.5 mg, 750.00 μmol, 1.5 eq.) was added to a mixture of 3C (92.1 mg, 0.5 mmol, 1 eq.) in THF (5 mL). The mixture was stirred at 50° C. under Ar atmosphere for 1 h. The mixture was cooled to rt, and then a solution of K₃PO₄ (530.7 mg, 2.50 mmol, 5 eq.) in H₂O (0.5 mL) was added. After stirring at rt for 0.5 h, 3-bromo-2-chloro-7-iodo-quinoline (Q2, 221.0 mg, 600.00 μmol, 1.2 eq.) and Pd(dppf)Cl₂ (36.6 mg, 50.00 μmol, 0.1 eq.) were added. The flask was degassed for several times and then stirred at 50° C. under Ar atmosphere for 11.5 h. The reaction progress was monitored by TLC (PE:EA=5:1). Upon completion, the mixture was diluted with brine (10 mL) and extracted with EA (2×20 mL). The combined organic layers were dried over anhydrous Na₂SO₄, filtered and concentrated to afford a residue. The residue was purified by silica gel chromatography (PE:EA=3:1) to afford 4C (146 mg, 342.14 μmol, 68% yield) as a yellow gum.

To a solution of 4C (0.2 g, 468.68 μmol, 1 eq.) in ACN (4 mL) was added IBX (196.86 mg, 703.02 μmol, 1.5 eq.) at 20° C., and the reaction was stirred at 60° C. for 2 h. The mixture was then cooled to 20° C. and filtered. The collected solid was washed with acetonitrile. The filtrate was concentrated under reduced pressure to afford a residue. The residue was purified by column chromatography (SiO₂, PE:EA=10:1 to 2:1) to give 5C (0.17 g, 400.27 μmol, 85.40% yield) as a white solid.

To a solution of 5C (200 mg, 470.90 μmol, 1 eq.) in THF (4 mL) was added LDA (2 M in THF, 0.3 mL, 1.27 eq.) at −78° C. The reaction was stirred at −78° C. for 0.5 h, and then dimethyl(methylene)ammonium iodide (5a) (348.48 mg, 1.88 mmol, 4 eq.) was added. The mixture was stirred at −78° C. for 0.5 h and then warmed to rt and stirred for 11.5 h. Then CH₃I (0.89 g, 6.27 mmol, 390.35 μL, 13.32 eq.) was added at rt, and the mixture was stirred for another 3 h. The reaction progress was monitored by TLC (PE:EA=3:1). Upon completion, the mixture was quenched by 10% NaHCO₃ (aq., 5 mL) and stirred for 0.5 h. The mixture was extracted with EA (2×50 mL). The combined organic layers were washed with 10% NaHCO₃ (aq., 20 mL), brine (20 mL), dried with anhydrous Na₂SO₄, filtered and concentrated in vacuum. The residue was purified by vacuum silica gel column chromatography (SiO₂, PE:EA=15:1 to 5:1) to give 6C (120 mg, 219.82 μmol, 46.68% yield, 80% purity) as a white solid.

To a solution of 6C (854 mg, 1.96 mmol, 1 eq.) in MeOH (10 mL) and THF (10 mL) was added CeCl₃.7H₂O (801.43 mg, 2.15 mmol, 204.45 μL, 1.1 eq.) at −78° C. The mixture was stirred at the same temperature for 10 min, then NaBH₄ (81.38 mg, 2.15 mmol, 1.1 eq.) was added. The mixture was stirred at −78° C. and stirred for another 10 min. The mixture was warmed to 0° C. and stirred at 0° C. for 10 min. The reaction was quenched with NH₄Cl (sat., aq., 5 mL), and the mixture was extracted with EA (2×50 mL). The combined organic layers were washed with brine (20 mL), dried with anhydrous Na₂SO₄, filtered and concentrated in vacuum to give a residue. The residue was purified by column chromatography (SiO₂, PE:EA=8:1 to 3:1) to give 7C (654 mg, 894.38 μmol, 45.7% yield, 60% purity) as a brown solid.

To a solution of triphenyl phosphine (896.73 mg, 3.42 mmol, 2.5 eq.), 4-chloro-7H-pyrrolo[2,3-d]pyrimidine (315.02 mg, 2.05 mmol, 1.5 eq.), and 7C (600 mg, 1.37 mmol, 1 eq.) in THF (12 mL) was added DIAD (636.02 mg, 3.15 mmol, 611.56 μL, 2.3 eq.) at 20° C. After stirring at rt for 5 h, the mixture was concentrated in vacuum to give a residue. The residue was purified by column chromatography (SiO₂, PE:EA=20:1 to 8:1) to give 8C (600 mg, 1.02 mmol, 74.8% yield, 98% purity) as a brown solid.

8C (500 mg, 870.63 μmol, 1 eq.) was dissolved in NH₃.H₂O (10 mL) and dioxane (10 mL) at 20° C. in an autoclave. The mixture was stirred at 140° C. for 48 h and then concentrated to give a brown residue. The residue was purified by column chromatography (SiO₂, DCM:MeOH=40:1 to 10:1) to give 9C (70 mg, 14.49% yield) and 9C-a (280 mg, 449.73 μmol, 51.6% yield, 86% purity).

To a solution of 9C (60 mg, 108.13 μmol, 1 eq.) in THF (3 mL) was added HCl (4 M, 1.50 mL eq.) at 20° C. The mixture was stirred at 20° C. for 4 h and then concentrated. The residue was neutralized by NH₃.H₂O to reach pH˜8 and then purified by prep-HPLC (basic condition, column: Phenomenex Gemini-NX 150*30 mm*5 um; mobile phase: [water (0.04% NH₃H₂O+10 mM NH₄HCO₃)-ACN]; B %: 31%-71%, 8 min) to give 3 (35 mg, 67.72 μmol, 62.6% yield, 99.6% purity) as a white solid. LCMS: (ESI): m/z calcd. for C₂₃H₂₂BrClN₅O₂, 516.1 [M+H]⁺, found 516.1. ¹H NMR (400 MHz, DMSO) δ: 8.92 (s, 1H), 8.03 (s, 1H), 7.98 (d, J=8.3 Hz, 1H), 7.88 (s, 1H), 7.68 (dd, J=1.5, 8.5 Hz, 1H), 7.12 (d, J=3.5 Hz, 1H), 6.95 (br s, 2H), 6.58 (d, J=3.5 Hz, 1H), 5.44 (br d, J=9.3 Hz, 1H), 5.03 (d, J=6.8 Hz, 1H), 4.98 (br s, 1H), 4.91 (d, J=3.5 Hz, 1H), 4.44-4.34 (m, 1H), 4.31 (br s, 1H), 3.96 (br s, 1H), 3.31-3.24 (m, 1H), 3.07-2.85 (m, 2H), 2.07-1.77 (m, 2H).

To a solution of 9C-a (80 mg, 149.41 μmol, 1 eq.) in THF (4.5 mL) was added HCl (4 M, 2.25 mL, 55.49 eq.) at 20° C. The mixture was stirred at 20° C. for 4 h and then concentrated. The residue was neutralized by NH₃.H₂O to reach pH˜8 and then purified by prep-HPLC (basic condition, column: Phenomenex Gemini-NX 150*30 mm*5 um; mobile phase: [water (0.04% NH₃H₂O+10 mM NH₄HCO₃)-ACN]; B %: 25%-55%, 8 min) to give 4 (45 mg, 89.48 μmol, 60% yield, 98.5% purity) as white solid. LCMS: (ESI): m/z calcd. for C₂₃H₂₄BrN₆O₂, 495.1, 497.1 [M+H]⁺, found 495.2, 497.2. ¹H NMR (400 MHz, CD₃OD) δ: 8.28 (s, 1H), 8.06 (s, 1H), 7.59 (d, J=8.3 Hz, 1H), 7.45 (s, 1H), 7.24 (d, J=8.0 Hz, 1H), 7.09 (d, J=3.5 Hz, 1H), 6.64 (d, J=3.8 Hz, 1H), 5.57 (br d, J=9.3 Hz, 1H), 5.10 (br s, 1H), 4.54 (s, 1H), 4.44 (dd, J=4.9, 9.2 Hz, 1H), 4.08 (br d, J=2.8 Hz, 1H), 3.07-2.85 (m, 2H), 2.69 (br s, 1H), 2.15-1.84 (m, 2H).

### Example 3

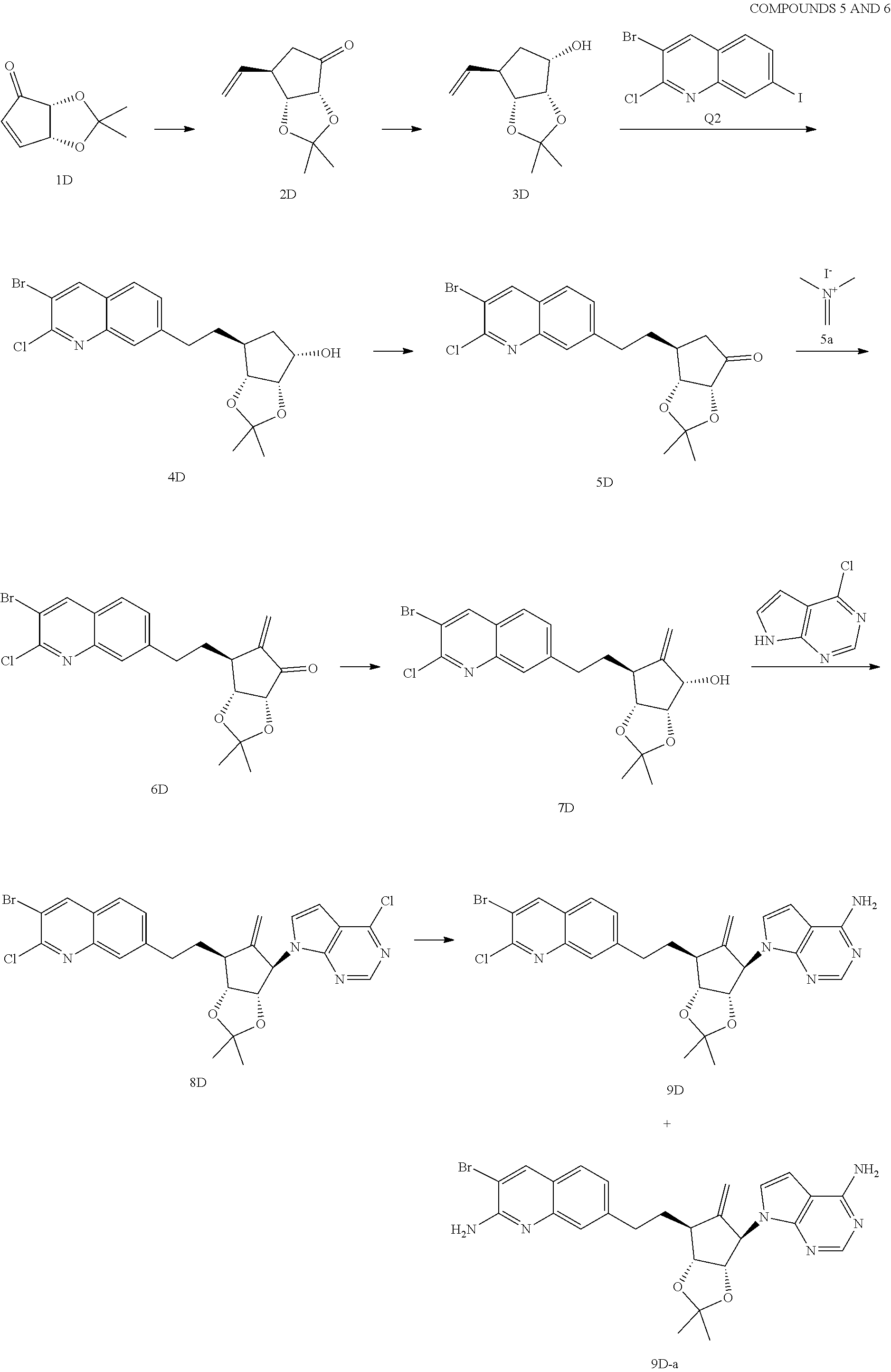

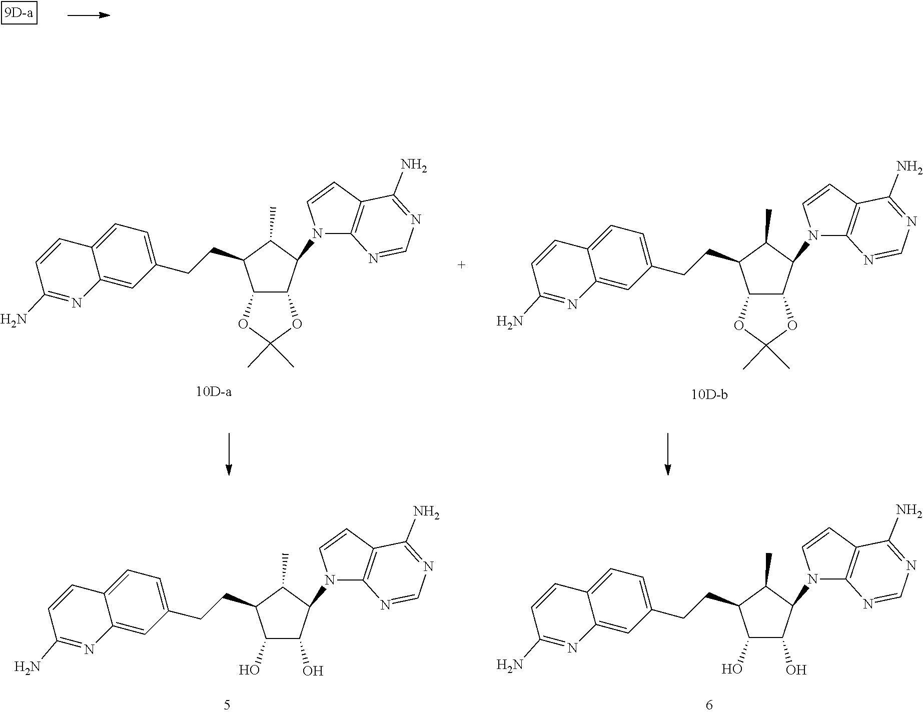 [SMILES: C[C@H]1[C@H](CCc2ccc3ccc(N)nc3c2)[C@H]2OC(C)(C)O[C@H]2[C@@H]1n1ccc2c(N)ncnc21 ; C[C@@H]1[C@H](CCc2ccc3ccc(N)nc3c2)[C@H]2OC(C)(C)O[C@H]2[C@@H]1n1ccc2c(N)ncnc21 ; C[C@H]1[C@H](CCc2ccc3ccc(N)nc3c2)[C@@H](O)[C@@H](O)[C@@H]1n1ccc2c(N)ncnc21 ; C[C@@H]1[C@H](CCc2ccc3ccc(N)nc3c2)[C@@H](O)[C@@H](O)[C@@H]1n1ccc2c(N)ncnc21]

TMEDA (5.65 g, 48.65 mmol, 7.34 mL, 1.5 eq.) was added to a mixture of CuI (339.73 mg, 1.78 mmol, 0.055 eq.) in THF (125 mL) at 0° C. The mixture was stirred at 0° C. for 5 min and then cooled to −78° C. A solution of vinylmagnesium bromide (1 M in THF, 48.65 mL, 1.5 eq.) was added, and the mixture was stirred at −78° C. for 20 min. TMSCl (4.23 g, 38.92 mmol, 4.94 mL, 1.2 eq.) was added, followed by a solution of ID (5 g, 32.43 mmol, 1 eq.) in THF (35 mL). The mixture was stirred at −78° C. for 3 h. The reaction progress was monitored by TLC (PE:EA=5:1). Upon completion, the reaction was quenched by NH₄Cl (sat., aq., 50 mL), and the mixture was extracted with EA (2×100 mL). The combined organic layers were washed with brine (2×100 mL), dried over Na₂SO₄, filtered and concentrated to give a residue. The residue was purified by column chromatography (SiO₂, PE:EA=20:1 to 10:1) to afford 2D (3.8 g, 18.77 mmol, 57% yield) as a yellow oil.

NaBH₄ (151.3 mg, 4.00 mmol, 2 eq.) was added to a mixture of 2D (364.4 mg, 2 mmol, 1 eq.) in MeOH (30 mL) at 0° C. The mixture was stirred at 0° C. for 0.5 h. The reaction progress was monitored by TLC (PE:EA=5:1). Upon completion, the mixture was quenched by acetone (5 mL) and then concentrated under reduced pressure to afford a residue. The residue was diluted with brine (50 mL) and then extracted with EA (2×50 mL). The combined organic layers were dried over anhydrous Na₂SO₄, filtered and concentrated to afford a crude product. The crude product was purified by silica gel chromatography (PE:EA=20:1) to afford 3D (260.0 mg, 1.41 mmol, 71% yield) as a colorless oil.

9-BBN dimer (181.5 mg, 750.00 μmol, 1.5 eq.) was added to a mixture of 3D (92.1 mg, 0.5 mmol, 1 eq.) in THF (5 mL). The mixture was stirred at 50° C. under Ar for 1 h. The mixture was cooled to rt, and then a solution of K₃PO₄ (530.7 mg, 2.50 mmol, 5 eq.) in H₂O (0.5 mL) was added. After stirring at rt for 0.5 h, Q2 (221.0 mg, 600.00 μmol, 1.2 eq.) and Pd(dppf)Cl₂ (36.6 mg, 50.00 μmol, 0.1 eq.) were added. The flask was degassed for several times and then stirred at 50° C. under Ar for 11.5 h. The reaction progress was monitored by TLC (PE:EA=5:1). Upon completion, the mixture was diluted with brine (10 mL) and extracted with EA (2×20 mL). The combined organic layers were dried over anhydrous Na₂SO₄, filtered and concentrated to afford a residue. The residue was purified by silica gel chromatography (PE:EA=3:1) to afford 4D (146 mg, 342.14 μmol, 68% yield) as a yellow gum.

To a solution of 4D (0.2 g, 468.68 μmol, 1 eq.) in ACN (4 mL) was added IBX (196.86 mg, 703.02 μmol, 1.5 eq.) at 20° C., and the mixture was stirred at 60° C. for 2 h. The mixture was then cooled to 20° C. and filtered. The collected solid was washed with ACN. The filtrate was concentrated under reduced pressure to afford a residue. The residue was purified by column chromatography (SiO₂, PE:EA=10:1 to 2:1) to give 5D (0.17 g, 400.27 μmol, 85.40% yield) as a white solid.

To a solution of 5D (200 mg, 470.90 μmol, 1 eq.) in THF (4 mL) was added LDA (2 M in THF, 0.3 mL, 1.27 eq.) at −78° C. The mixture was stirred at −78° C. for 0.5 h, and then dimethyl(methylene)ammonium iodide (5a) (348.48 mg, 1.88 mmol, 4 eq.) was added. The mixture was stirred at −78° C. for 0.5 h. The mixture was then warmed to rt and stirred for 11.5 h. CH₃I (0.89 g, 6.27 mmol, 390.35 μL, 13.32 eq.) was added at rt, and the mixture was stirred for another 3 h. The reaction progress was monitored by TLC (PE:EA=3:1). Upon completion, the reaction was quenched by 10% NaHCO₃ (aq., 5 mL) and stirred for 0.5 h. The mixture was extracted with EA (2×50 mL). The combined organic layers were washed with 10% NaHCO₃ (aq., 20 mL) and brine (20 mL), dried with anhydrous Na₂SO₄, filtered and concentrated in vacuum. The residue was purified by vacuum silica gel column chromatography (SiO₂, PE:EA=15:1 to 5:1) to give 6D (120 mg, 219.82 μmol, 46.7% yield, 80% purity) as a white solid.

To a solution of 6D (854 mg, 1.96 mmol, 1 eq.) in MeOH (10 mL) and THF (10 mL) was added CeCl₃.7H₂O (801.43 mg, 2.15 mmol, 204.45 μL, 1.1 eq.) at −78° C. The mixture was stirred at the same temperature for 10 min. NaBH₄ (81.38 mg, 2.15 mmol, 1.1 eq.) was added at −78° C., and the mixture was stirred for 10 min. The mixture was warmed to 0° C. and then stirred at 0° C. for 10 min. The reaction was quenched with NH₄Cl (sat., aq., 5 mL), and the mixture was extracted with EA (2×50 mL). The combined organic layers were washed with brine (20 mL), dried with anhydrous Na₂SO₄, filtered and concentrated in vacuum to give a residue. The residue was purified by column chromatography (SiO₂, PE:EA=8:1 to 3:1) to give 7D (654 mg, 894.38 μmol, 45.7% yield, 60% purity) as a brown solid.

To a solution of triphenyl phosphine (896.73 mg, 3.42 mmol, 2.5 eq.), 4-chloro-7H-pyrrolo[2,3-d]pyrimidine (315.02 mg, 2.05 mmol, 1.5 eq.), and 7D (600 mg, 1.37 mmol, 1 eq.) in THF (12 mL) was added DIAD (636.02 mg, 3.15 mmol, 611.56 μL, 2.3 eq.) at 20° C. After stirring at rt for 5 h, the mixture was concentrated in vacuum to give a residue. The residue was purified by column chromatography (SiO₂, PE:EA=20:1 to 8:1) to give 8D (600 mg, 1.02 mmol, 74.8% yield, 98% purity) as a brown solid.

8D (500 mg, 870.63 μmol, 1 eq.) was dissolved in NH₃.H₂O (10 mL) and dioxane (10 mL) at 20° C. in an autoclave. The mixture was stirred at 140° C. for 48 h and then concentrated to give a brown residue. The residue was purified by column chromatography (SiO₂, DCM:MeOH=40:1 to 10:1) to give 9D (70 mg, 14.5% yield) and 9D-a (280 mg, 449.73 μmol, 51.6% yield, 86% purity).

To a solution of 9D-a (45 mg, 84.04 μmol) in EtOH (4 mL) was added PtO₂ (3.82 mg, 16.81 μmol, 0.2 eq.) at rt. The mixture was degassed under vacuum and purged with H₂ (15 psi) several times. The mixture was stirred under H₂ (15 psi) at rt for 6 h and then filtered over a pad of Celite. The filtrate was concentrated to give a residue. The residue was purified by column chromatography (SiO₂, DCM:MeOH=20:1 to 7:1) to give a mixture of 10D-a and 10D-b (35 mg) as a white solid. The ratio of isomers was approximately 2:1 based on SFC analysis. The mixture was purified by SFC separation (column: DAICEL CHIRALCELOD (250 mm*30 mm, 10 um); mobile phase: [0.1% NH₃H₂O ETOH]; B %: 45%) to give 10D-a (retention time: 3.471 min) (20 mg, 43.62 μmol, 57.1% yield) as a white solid and 10D-b (retention time: 2.779 min) (14 mg, 30.53 μmol, 40.0% yield) as a white solid. 10D-a: ¹H NMR (400 MHz, CD₃OD) δ: 8.04 (s, 1H), 7.87 (d, J=9.0 Hz, 1H), 7.55 (d, J=8.1 Hz, 1H), 7.41 (s, 1H), 7.27-7.13 (m, 2H), 6.75 (d, J=8.8 Hz, 1H), 6.62 (d, J=3.4 Hz, 1H), 4.94-4.89 (m, 1H), 4.61-4.46 (m, 2H), 3.14-2.92 (m, 1H), 2.83 (br dd, J=8.8, 14.2 Hz, 1H), 2.26 (dt, J=6.6, 11.4 Hz, 1H), 2.09-1.97 (m, 2H), 1.54 (s, 3H), 1.57-1.51 (m, 1H), 1.30 (s, 3H), 0.81 (br d, J=6.6 Hz, 3H). LCMS: (ESI): m/z calcd. for C₂₆H₃₁N₆O₂, 459.2 [M+H]⁺, found 459.2. 10D-b: ¹H NMR (400 MHz, CD₃OD) δ: 8.04 (s, 1H), 7.86 (d, J=8.8 Hz, 1H), 7.54 (d, J=8.3 Hz, 1H), 7.37 (s, 2H), 7.14 (dd, J=1.5, 8.1 Hz, 1H), 6.74 (d, J=8.8 Hz, 1H), 6.60 (d, J=3.7 Hz, 1H), 5.38 (t, J=7.5 Hz, 1H), 4.99 (t, J=6.7 Hz, 1H), 4.59 (t, J=7.1 Hz, 1H), 2.90-2.66 (m, 3H), 2.39-2.22 (m, 1H), 1.94-1.80 (m, 2H), 1.49 (s, 3H), 1.35 (s, 3H), 0.65 (d, J=7.6 Hz, 3H). LCMS: (ESI): m/z calcd. for C₂₆H₃₁N₆O₂, 459.2 [M+H]⁺, found 459.2.

To a solution of 10D-a (20 mg, 43.62 μmol, 1 eq.) in THF (3 mL) was added HCl (4 M, 1.5 mL in H₂O) at rt. The mixture was stirred at rt for 12 h. The mixture was then concentrated in vacuum to give a residue. The mixture was neutralized with NH₃.H₂O to pH 9. The residue was purified by prep-HPLC (basic condition; column: Phenomenex Gemini-NX 150*30 mm*5 um; mobile phase: [water (0.04% NH₃H₂O+10 mM NH₄HCO₃)-ACN]; B %: 20%-50%, 8 min) to give 5 (12 mg, 28.53 μmol, 65.4% yield, 99.5% purity) as a white solid. ¹H NMR (400 MHz, CD₃OD) δ: 8.04 (s, 1H), 7.88 (d, J=8.8 Hz, 1H), 7.56 (d, J=8.0 Hz, 1H), 7.40 (s, 1H), 7.27-7.13 (m, 2H), 6.75 (d, J=9.0 Hz, 1H), 6.61 (d, J=3.5 Hz, 1H), 4.58-4.45 (m, 1H), 4.36 (dd, J=6.3, 9.0 Hz, 1H), 3.97 (dd, J=3.9, 6.1 Hz, 1H), 3.04-2.77 (m, 2H), 2.06-1.77 (m, 3H), 1.71-1.60 (m, 1H), 0.93 (d, J=6.5 Hz, 3H). LCMS: (ESI): m/z calcd. for C₂₃H₂₇N₆O₂, 419.5 [M+H]⁺, found 419.2.

To a solution of 10D-b (14 mg, 30.53 μmol, 1 eq.) in THF (3 mL) was added HCl (4 M, 1.5 mL in water) at 20° C. The mixture was stirred at 20° C. for 12 h. The mixture was concentrated in vacuum to give a residue. The residue was neutralized with NH₃.H₂O to pH 9. The residue was purified by prep-HPLC (basic condition; column: Phenomenex Gemini-NX 150*30 mm*5 um; mobile phase: [water (0.04% NH₃H₂O+10 mM NH₄HCO₃)-ACN]; B %: 20%-50%, 8 min) to give 6 (7 mg, 16.54 μmol, 54.17% yield, 98.88% purity) as a white solid. ¹H NMR (400 MHz, CD₃OD) δ: 8.06 (s, 1H), 7.87 (d, J=8.8 Hz, 1H), 7.56 (d, J=7.8 Hz, 1H), 7.38 (s, 1H), 7.32 (d, J=3.8 Hz, 1H), 7.20-7.09 (m, 1H), 6.75 (d, J=8.8 Hz, 1H), 6.60 (d, J=3.8 Hz, 1H), 4.86 (br s, 1H), 4.76-4.68 (m, 1H), 3.95 (t, J=7.3 Hz, 1H), 2.84 (t, J=8.2 Hz, 2H), 2.71-2.57 (m, 1H), 2.24-2.09 (m, 1H), 2.08-1.78 (m, 2H), 0.58 (d, J=7.5 Hz, 3H). LCMS: (ESI): m/z calcd. for C₂₃H₂₇N₆O₂, 419.5 [M+H]⁺, found 419.2.

### Example 4

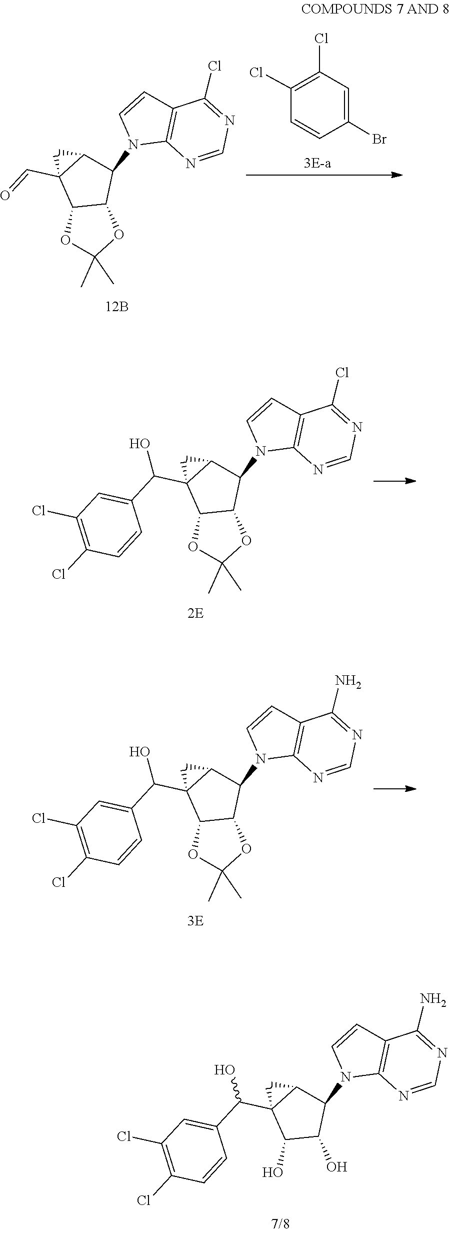 [SMILES: CC1(C)O[C@H]2[C@H](n3ccc4c(Cl)ncnc43)[C@H]3C[C@@]3(C=O)[C@H]2O1 ; Clc1ccc(Br)cc1Cl ; CC1(C)O[C@H]2[C@H](n3ccc4c(Cl)ncnc43)[C@H]3C[C@@]3(C(O)c3ccc(Cl)c(Cl)c3)[C@H]2O1 ; CC1(C)O[C@H]2[C@H](n3ccc4c(N)ncnc43)[C@H]3C[C@@]3(C(O)c3ccc(Cl)c(Cl)c3)[C@H]2O1 ; Nc1ncnc2c1ccn2[C@H]1[C@H](O)[C@H](O)[C@]2(C(O)c3ccc(Cl)c(Cl)c3)C[C@H]12]

Chloro(isopropyl)magnesium (2 M, 449.41 μL, 2 eq.) was added dropwise to a solution of 4-bromo-1,2-dichloro-benzene (203.04 mg, 898.83 μmol, 2 eq.) in THF (1 mL) at −15 C. The mixture was stirred at −15° C. for 10 min, and then warmed to 0° C. The mixture was stirred at 0° C. for 1 h. A solution of 12B (150 mg, 449.41 μmol, 1 eq.) in THF (1.5 mL) was added at −20° C., and the mixture was stirred at 0° C. for 20 min. The reaction progress was monitored by TLC (PE:EA=2:1). Upon completion, the reaction was quenched with NH₄Cl (sat. aq., 2 mL) and extracted with EA (2×3 mL). The combined organic layers were washed with brine (2×3 mL), dried over Na₂SO₄, and concentrated to give a residue. The residue was purified by column chromatography (SiO₂, PE:EA=20:1 to 6:1) to afford two isomers, (R or S)-[(15S,16R,17S,18R,21R)-16-(4-chloropyrrolo[2,3-d]pyrimidin-7-yl)-20,20-dimethyl-26,27-dioxatricyclononan-21-yl]-(3,4-dichlorophenyl)methanol (2E-a) (60 mg, 92.35 μmol, 20% yield) as a white solid, and (S or R)-[(15S,16R,17S,18R,21R)-16-(4-chloropyrrolo[2,3-d]pyrimidin-7-yl)-20,20-dimethyl-26,27-dioxatricyclononan-21-yl]-(3,4-dichlorophenyl)methanol (2E-b) (70 mg, 110.66 μmol, 24% yield) as a white solid.

Compound 2E-a: LCMS: (ESI): m/z calcd. for C₂₂H₂₁Cl₃N₃O₃ 482.05 [M+H]⁺, found 481.7. ¹H NMR (400 MHz, CDCl₃) δ: 8.70 (s, 1H), 7.70 (s, 1H), 7.48-7.35 (m, 2H), 7.30 (d, J=3.5 Hz, 1H), 6.66 (d, J=3.3 Hz, 1H), 5.84 (s, 1H), 5.49 (d, J=7.3 Hz, 1H), 4.74 (s, 1H), 4.68 (d, J=7.5 Hz, 1H), 4.44 (s, 1H), 1.94 (br dd, J=4.7, 9.2 Hz, 1H), 1.46 (s, 3H), 1.32-1.28 (m, 2H), 1.15 (s, 3H). Compound 2E-b: LCMS: (ESI): m/z calcd. for C₂₂H₂₁Cl₃N₃O₃ 482.05 [M+H]⁺, found 481.9. ¹H NMR (400 MHz, CDCl₃) δ: 8.72 (s, 1H), 7.39-7.32 (m, 2H), 7.26 (br s, 1H), 7.09 (dd, J=1.5, 8.2 Hz, 1H), 6.66 (d, J=3.5 Hz, 1H), 5.68 (d, J=7.3 Hz, 1H), 5.59 (d, J=6.2 Hz, 1H), 5.35 (d, J=6.2 Hz, 1H), 4.81 (s, 1H), 4.67 (d, J=7.5 Hz, 1H), 1.66 (br dd, J=4.4, 9.3 Hz, 2H), 1.59-1.57 (m, 6H), 1.10 (s, 1H), 0.56 (br dd, J=6.5, 8.5 Hz, 1H).

NH₃.H₂O (728.00 mg, 5.82 mmol, 0.8 mL, 28% purity, 46.61 eq.) was added to a solution of (2E-a) (60 mg, 124.80 μmol, 1 eq.) in dioxane (1.2 mL). The mixture was stirred at 100° C. for 16 h. The reaction progress was monitored by TLC (PE:EA=1:1). Upon completion, the mixture was extracted with EA (3×5 mL). The combined organic layers were washed with brine (3×5 mL), dried over Na₂SO₄, and concentrated to give a residue that afforded (R or S)-[(15S,16R,17S,18R,21R)-16-(4-aminopyrrolo[2,3-d]pyrimidin-7-yl)-20,20-dimethyl-27,28-dioxatricyclononan-21-yl]-(3,4-dichlorophenyl)methanol (3E-a) (68 mg, crude) as a pale yellow solid. LCMS: (ESI): m/z calcd. for C₂₂H₂₃Cl₂N₄O₃ 461.11 [M+H]⁺, found 461.0.

(S or R)-[(15S,16R,17S,18R,21R)-16-(4-aminopyrrolo[2,3-d]pyrimidin-7-yl)-20,20-dimethyl-27,28-dioxatricyclononan-21-yl]-(3,4-dichlorophenyl)methanol (3E-b) (67 mg, crude) was obtained as a pale yellow solid by the same procedure from (S or R)-[(15S,16R,17S,18R,21R)-16-(4-chloropyrrolo[2,3-d]pyrimidin-7-yl)-20,20-dimethyl-26,27-dioxatricyclononan-21-yl]-(3,4-dichlorophenyl)methanol (2E-b) (70 mg, 145.60 μmol, 1 eq.). LCMS: (ESI): m/z calcd. for C₂₂H₂₃Cl₂N₄O₃ 461.11 [M+H]⁺, found 461.1.

HCl (4 M, aq., 0.75 mL, 20.35 eq.) was added to a solution of (3E-a) (68 mg, 147.40 μmol, 1 eq.) in THF (1.5 mL). The mixture was stirred at 25° C. for 16 h. The reaction progress was monitored by TLC (DCM:MeOH=10:1). Upon completion, the reaction mixture was quenched by NH₄OH (25% wt, 1 mL) and then concentrated to give a residue. The residue was purified by prep-HPLC (column: Phenomenex Gemini-NX 150*30 mm*5 um; mobile phase: [water (0.04% NH₃H₂O+10 mM NH₄HCO₃)-ACN]; B %: 24%-54%, 8 min) and SFC (column: DAICEL CHIRALPAK AD (250 mm*30 mm, 10 um); mobile phase: [0.1% NH₃H₂O ETOH]; B %: 45%) to afford (1R,2R,3S,4R,5S)-4-(4-aminopyrrolo[2,3-d]pyrimidin-7-yl)-1-[(R or S)-(3,4-dichlorophenyl)-hydroxy-methyl]bicyclo[3.1.0]hexane-2,3-diol (7) (15 mg, 35.29 μmol, 99% purity) as a white solid. LCMS: (ESI): m/z calcd. for C₁₉H₁₉Cl₂N₄O₃ 421.08 [M+H]⁺, found 421.0. ¹H NMR (400 MHz, MeOD) δ: 8.09 (s, 1H), 7.69 (s, 1H), 7.46 (q, J=8.4 Hz, 2H), 7.24 (d, J=3.3 Hz, 1H), 6.57 (d, J=3.3 Hz, 1H), 4.83 (br s, 1H), 4.74 (br d, J=6.5 Hz, 1H), 4.58 (s, 1H), 3.88 (br d, J=6.5 Hz, 1H), 1.62 (br d, J=9.3 Hz, 1H), 1.48 (br s, 1H), 1.16-1.08 (m, 1H).

(1R,2R, 3S, 4R, 5S)-4-(4-aminopyrrolo[2,3-d]pyrimidin-7-yl)-1-[(S or R)-(3,4-dichlorophenyl)-hydroxy-methyl]bicyclo[3.1.0]hexane-2,3-diol (8) (19 mg, 44.65 μmol, 99% purity) was obtained as a white solid by the same procedure and purified by prep-HPLC (column: Phenomenex Gemini-NX 150*30 mm*5 um; mobile phase: [water (0.04% NH₃H₂O+10 mM NH₄HCO₃)-ACN]; B %: 24%-54%, 8 min) and SFC (column: DAICEL CHIRALPAK AD (250 mm*30 mm, 10 um); mobile phase: [0.1% NH₃H₂O ETOH]; B %: 45%) from (S or R)-[(15S,16R,17S,18R,21R)-16-(4-aminopyrrolo[2,3-d]pyrimidin-7-yl)-20,20-dimethyl-27,28-dioxatricyclononan-21-yl]-(3,4-dichlorophenyl)methanol (3E-b) (67 mg, crude). Compound 8: LCMS: (ESI): m/z calcd. for C₁₉H₁₉Cl₂N₄O₃ 421.08 [M+H]⁺, found 421.1. ¹H NMR (400 MHz, CD₃OD) (signals under solvent peak not listed) δ: 8.08 (br s, 1H), 7.54-7.45 (m, 2H), 7.42 (br s, 1H), 7.31 (br s, 1H), 6.58 (br s, 1H), 5.17 (br s, 1H), 3.78 (br s, 1H), 1.52-1.39 (m, 2H), 0.38 (br s, 1H).

### Example 5

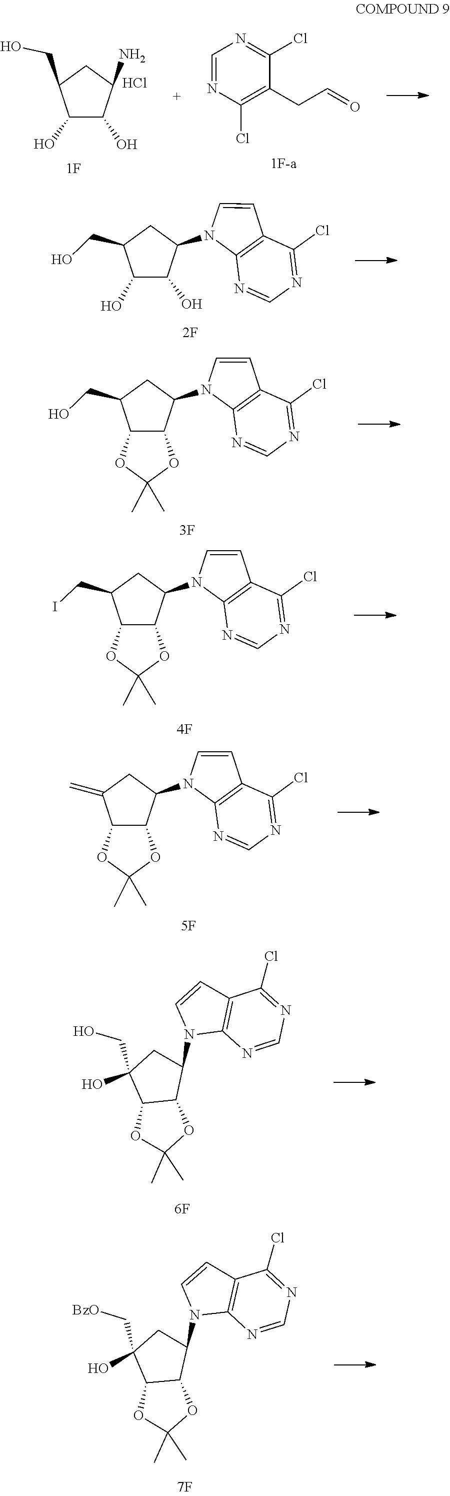

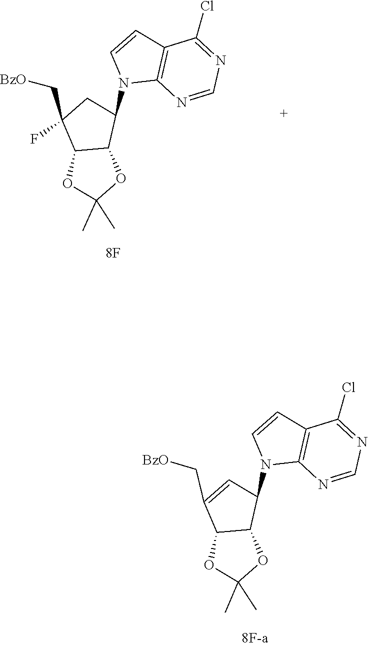

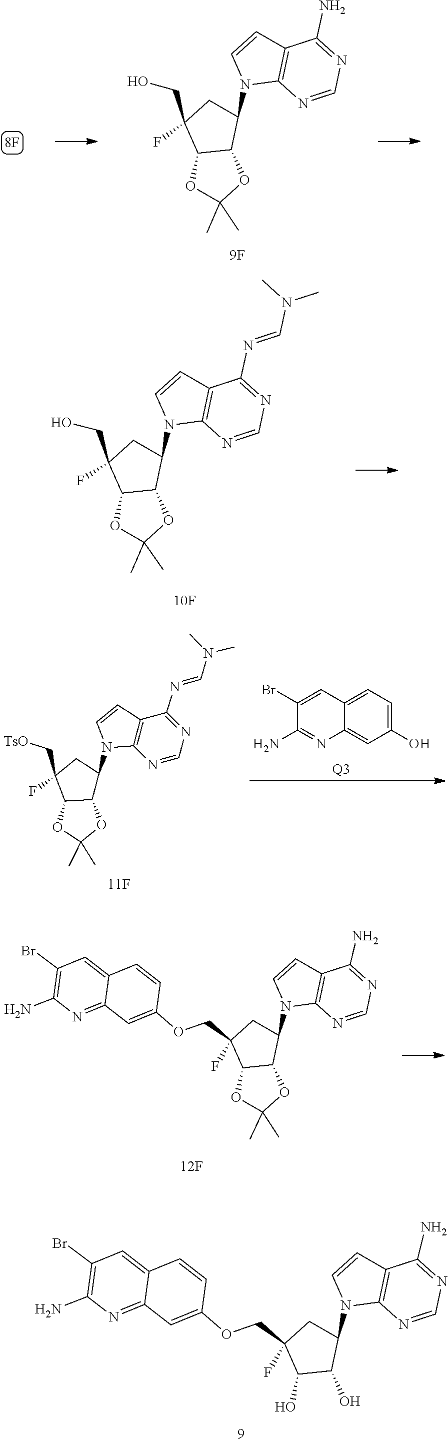

To a solution of (1R,2S,3R,5R)-3-amino-5-(hydroxymethyl)cyclopentane-1,2-diol (1F) (3.67 g, 20.0 mmol, 1 eq., HCl salt) and 2-(4,6-dichloropyrimidin-5-yl)acetaldehyde (1F-a) (3.82 g, 20.0 mmol, 1 eq.) in EtOH (80 mL) was added TEA (6.07 g, 60 mmol, 8.4 mL, 3 eq.). The mixture was refluxed for 24 h. The reaction progress was monitored by LCMS. Upon completion, the mixture was concentrated under vacuum to give a residue. The residue was dissolved in sat. NaHCO₃ solution (50 mL), and then extracted with EtOAc (9×50 mL). The combined organic layer were washed with brine (300 mL), dried over Na₂SO₄, and then filtered. The filtrate was concentrated under reduced pressure to afford the crude 2F (5.8 g) as a yellow gum, which was used for next step without further purification.

To a solution of 2F (5.8 g, crude) and 2,2-dimethoxypropane (4.26 g, 40.8 mmol, 5.0 mL, 2 eq.) in acetone (60 mL) was added 4-methylbenzenesulfonic acid hydrate (388.9 mg, 2.0 mmol, 0.1 eq.). The mixture was stirred at rt for 2 h and then refluxed for 24 h. The reaction progress was monitored by LCMS. Upon completion, the reaction was quenched by Et₃N (0.7 mL), and then concentrated under reduced pressure to give a residue. The residue was treated with sat. NaHCO₃ solution (20 mL) and brine (20 mL). The resulting mixture was extracted with EtOAc (3×40 mL). The combined organic layers were washed with brine (100 mL), dried over Na₂SO₄, and then filtered. The filtrate was concentrated under reduced pressure to give a residue. The residue was purified by column chromatography (PE:EA=5:1 to 1:1) to afford [(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]methanol (3F) (4.25 g, 65% yield, 2 steps from 1F) as a yellow solid. LCMS: (ESI): m/z calcd. for C₁₅H₁₉ClN₃O₃ 324.11 [M+H]⁺, found 324.2.

To a solution of PPh₃ (6.72 g, 25.6 mmol, 2 eq.) and imidazole (1.83 g, 26.9 mmol, 2.1 eq.) in THF (60 mL) was added I₂ (6.51 g, 25.6 mmol, 2 eq.). The mixture was stirred at rt for 15 min under N₂, and then a solution of 3F (4.15 g, 12.8 mmol, 1 eq.) in THF (40 mL) was added. The mixture was stirred at rt for 1.5 h under N₂. The reaction progress was monitored by TLC (PE:E=5:1). Upon completion, the reaction was quenched with sat. Na₂S₂O₃ solution (100 mL), and then extracted with DCM (3×100 mL). The combined organic layers were washed with brine (300 mL), dried over Na₂SO₄, and then filtered. The filtrate was concentrated in vacuum to give a residue. The residue was purified by column chromatography (PE:EA=20:1 to 5:1) to afford 7-[(3aS,4R,6S,6aR)-6-(iodomethyl)-2,2-dimethyl-4,5,6,6a-tetrahydro-3aH-cyclopenta[d][1,3]dioxol-4-yl]-4-chloro-pyrrolo[2,3-d]pyrimidine (4F) (4.16 g, 70% yield) as a yellow solid. LCMS: (ESI): m/z calcd. for C₁₅H₁₈ClIN₃O₂ 434.01 [M+H]⁺, found 434.0.

tBuOK (697.0 mg, 6.2 mmol, 1 eq.) in THF (8 mL) was added dropwise to a mixture of 4F (2.69 g, 6.2 mmol, 1 eq.) and THF (30 mL) at 0° C. The mixture was stirred at 0° C. for 0.5 h. The reaction progress was monitored by TLC (PE:EA=5:1). Upon completion, the reaction was quenched with sat. aq. NH₄Cl (50 mL). The mixture was extracted with EA (2×50 mL). The separated organic layers were combined and washed with brine (30 mL), dried over anhydrous Na₂SO₄ and concentrated to afford a residue. The residue was purified by silica gel chromatography (EA:PE=8:1) to afford 7-[(3aR,6R,6aS)-2,2-dimethyl-4-methylene-3a,5,6,6a-tetrahydrocyclopenta[d][1,3]dioxol-6-yl]-4-chloro-pyrrolo[2,3-d]pyrimidine (5F) (1.9 g, 94% yield) as colorless foam. LCMS: (ESI): m/z calcd. for C₁₅H₁₇ClN₃O₂ 306.10 [M+H]⁺, found 306.1.

K₂OsO₄.2H₂O (75.32 mg, 204.41 μmol, 0.025 eq.) was added to a mixture of 5F (2.5 g, 8.2 mmol, 1 eq.) and NMO (1.9 g, 16.4 mmol, 2 eq.) in a mixed solvent of acetone (40 mL) and H₂O (8 mL). The mixture was stirred at rt for 20 h. The reaction progress was monitored by TLC (PE:EA=5:1). Upon completion, the mixture was concentrated under reduced pressure to afford a residue. The residue was purified by silica gel chromatography (PE:EA:EtOH=30:10:1) to afford (3aS,4R,6R,6aS)-6-(4-chloropyrrolo[2,3-d]pyrimidin-7-yl)-4-(hydroxymethyl)-2,2-dimethyl-3a,5,6,6a tetrahydrocyclopenta[d][1,3]dioxol-4-ol (6F) (2.51 g, 90% yield) as a light yellow foam. LCMS: (ESI): m/z calcd. for C₁₅H₁₉ClN₃O₄ 340.11 [M+H]⁺, found 340.0.

Benzoyl chloride (297.87 mg, 2.12 mmol, 246.17 μL, 1.2 eq.) was added dropwise to a mixture of 6F (600 mg, 1.77 mmol, 1 eq.), DMAP (21.6 mg, 176.6 μmol, 0.1 eq.), and Et₃N (536.1 mg, 5.3 mmol, 737.4 μL, 3 eq.) in DCM (10 mL) at rt. The mixture was stirred at rt for 3 h. The reaction progress was monitored by LCMS. Upon completion, the mixture was concentrated under reduced pressure to afford a residue. The residue was purified by silica gel chromatography (EA:PE=3:1) to afford [(3aS,4R,6R,6aS)-6-(4-chloropyrrolo[2,3-d]pyrimidin-7-yl)-4-hydroxy-2,2-dimethyl-3a,5,6,6a-tetrahydrocyclopenta[d][1,3]dioxol-4-yl]methyl benzoate (7F) (743 mg, 94% yield) as a white foam. LCMS: (ESI): m/z calcd. for C₂₂H₂₃ClN₃O₅ 444.13 [M+H]⁺, found 444.1.

DAST (435.76 mg, 2.70 mmol, 357.18 μL, 2 eq.) was added to a solution of 7F (600 mg, 1.35 mmol, 1 eq.) in DCM (10 mL) at 0° C. The mixture was stirred at 0° C. for 1 h. The reaction progress was monitored by LCMS. Upon completion, the reaction was quenched with sat. NaHCO₃ (aq., 10 mL). The mixture was extracted with DCM (2×20 mL). The separated organic layers were combined and washed with brine (10 mL), dried over anhydrous Na₂SO₄ and concentrated to afford a residue. The residue was purified by chromatography on silica gel (PE:EA=8:1) to afford a mixture of 8F and 8F-a (200 mg, 8:8a=1:2) as a white foam. 8F: LCMS (ESI): m/z calcd. for C₂₂H₂₂ClFN₃O₄ 446.13 [M+H]⁺, found 446.2; and 8F-a: LCMS (ESI): m/z calcd. for C₂₂H₂₁ClN₃O₄ 426.12 [M+H]⁺, found 426.2.

A mixture of 8F:8a-F (=1:2, 50.00 mg) and NH₃.H₂O (1.7 mL, 25% wt) in dioxane (10 mL) was heated at 100° C. for 16 h. The mixture was concentrated under reduced pressure to afford a residue. The residue was purified by silica gel chromatography (DCM:MeOH=20:1) to afford the crude 9F as colorless gum (crude, 32 mg). LCMS: (ESI): m/z calcd. for C₁₅H₂₀FN₄O₃ 323.15 [M+H]⁺, found 323.2.

A mixture of 9F (crude 90 mg), DMF-DMA (133.1 mg, 1.1 mmol, 148.4 μL, 4 eq.) and THF (2 mL) was stirred at 60° C. for 48 h. The reaction progress was monitored by LCMS. Upon completion, the mixture was concentrated to afford crude 10F (crude, 90 mg) as a yellow gum, which was used for next step without further purification.

p-TsCl (159.12 mg, 834.63 μmol, 3 eq.) was added to a mixture of 10F (105 mg, 278.2 μmol, 1 eq.), TEA (140.8 mg, 1.4 mmol, 194 μL, 5 eq.), DMAP (3.4 mg, 27.8 μmol, 0.1 eq.) and DCM (1 mL). The mixture was stirred at 15° C. for 2 h. The reaction progress was monitored by TLC (DCM:MeOH=10:1). Upon completion, the mixture was concentrated to afford a residue. The residue was purified by silica gel chromatography (DCM:MeOH=30:1) to afford [(3aS,4S,6R,6aS)-6-[4-[(E)-dimethylaminomethyleneamino]pyrrolo[2,3-d]pyrimidin-7-yl]-4-fluoro-2,2-dimethyl-3a,5,6,6a-tetrahydrocyclopenta[d][1,3]dioxol-4-yl]methyl 4-methylbenzenesulfonate (11F) (58 mg) as a yellow foam. LCMS: (ESI): m/z calcd. for C₂₅H₃₁FN₅O₅S 532.20 [M+H]⁺, found 532.1.

A mixture of 11F (58 mg, 109.10 μmol, 1 eq.), 2-amino-3-bromo-quinolin-7-ol (Q3) (39.1 mg, 163.7 μmol, 1.5 eq.) and Cs₂CO₃ (106.7 mg, 327.3 μmol, 3 eq.) in DMF (1 mL) was stirred at 70° C. for 16 h. The reaction progress was monitored by LCMS. Upon completion, the mixture was diluted with water (10 mL). The resulting mixture was extracted with EA (2×20 m). The separated organic layers were combined and washed with brine (10 mL), dried over anhydrous Na₂SO₄, and concentrated to afford a residue. The residue was purified by silica gel chromatography (DCM:MeOH:Et₃N=30:1:0.3) to afford the crude product (30 mg). The crude product was further purified by chiral-SFC (column: DAICEL CHIRALCEL OD-H (250 mm*30 mm, 5 um); mobile phase: [0.1% NH₃H₂O ETOH]; B %: 45%) to afford 7-[[(3aS,4S,6R,6aS)-6-(4-aminopyrrolo[2,3-d]pyrimidin-7-yl)-4-fluoro-2,2-dimethyl-3a,5,6,6a-tetrahydrocyclopenta[d][1,3]dioxol-4-yl]methoxy]-3-bromo-quinolin-2-amine (12F) (18 mg, 26% yield) as a white solid. LCMS: (ESI): m/z calcd. for C₂₄H₂₅BrFN₆O₃ 543.12 [M+H]⁺, found 543.2.

HCl (4 M, 1 mL, 120.75 eq.) was added to the mixture of 12F (18 mg, 33.1 μmol, 1 eq.) and THF (2 mL) at rt. The mixture was stirred at rt, and the reaction progress was monitored by LCMS. Upon completion, the mixture was concentrated under reduced pressure to afford a residue. NH₃.H₂O (0.1 mL) was added to neutralize the residue. The resultant mixture was dissolved in MeCN:H₂O=1:1 (3 mL) and then purified by prep-HPLC (column: Phenomenex Gemini-NX 150*30 mm*5 um; mobile phase: [water (0.04% NH₃H₂O+10 mM NH₄HCO₃)-ACN]; B %: 27%-57%, 8 min) to afford (1S,2S,3S,5R)-3-[(2-amino-3-bromo-7-quinolyl)oxymethyl]-5-(4-aminopyrrolo[2,3-d]pyrimidin-7-yl)-3-fluoro-cyclopentane-1,2-diol (9) (9 mg, 54% yield) as white solid. LCMS: (ESI): m/z calcd. for C₂₁H₂₁BrFN₆O₃ 503.08 [M+H]⁺, found 503.1. ¹H NMR (400 MHz, CD₃OD) δ: 8.22 (s, 1H), 8.04 (s, 1H), 7.56 (d, J=9.0 Hz, 1H), 7.24 (d, J=3.7 Hz, 1H), 7.07 (d, J=2.2 Hz, 1H), 7.00 (dd, J=2.4, 8.8 Hz, 1H), 6.59 (d, J=3.7 Hz, 1H), 5.19-5.06 (m, 1H), 4.60-4.56 (m, 1H), 4.53-4.35 (m, 3H), 2.76-2.45 (m, 2H).

### Example 6

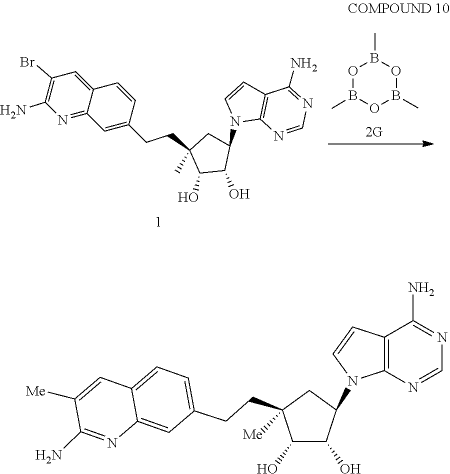

A mixture of 1 (20.1 mg, 40.4 μmol, 1 eq.), 2,4,6-trimethyl-1,3,5,2,4,6-trioxatriborinane (15.2 mg, 121.1 μmol, 16.9 μL, 3 eq.), K₃PO₄ (25.7 mg, 121.1 μmol, 3 eq.) and Pd(dppf)Cl₂ (2.9 mg, 4.0 μmol, 0.1 eq.) in a mixed solvent of dioxane (1 mL) and water (0.2 mL) was stirred at 90° C. under Ar for 16 h. The reaction progress was monitored by LCMS. Upon completion, the mixture was filtered through a filter element. The filtrate was purified by acid pre-HPLC (column: Venusil ASB Phenyl 150*30 mm*5 um; mobile phase: [water (0.05% HCl)-ACN]; B %: 15%-45%, 10 min) and then by basic pre-HPLC (column: Phenomenex Gemini-NX 150*30 mm*5 um; mobile phase: [water (0.04% NH₃H₂O+10 mM NH₄HCO₃)-ACN]; B %: 25%-55%, 8 min) to afford (1S,2R,3S,5R)-3-[2-(2-amino-3-methyl-7-quinolyl)ethyl]-5-(4-aminopyrrolo[2,3-d]pyrimidin-7-yl)-3-methyl-cyclopentane-1,2-diol (10) (5.4 mg, 31% yield) as a white solid. LCMS: (ESI): m/z calcd. for C₂₄H₂₉N₆O₂ 433.23 [M+H]⁺, found 433.3. ¹H NMR (400 MHz, CD₃OD) δ: 8.08 (s, 1H), 7.74 (s, 1H), 7.54 (d, J=8.1 Hz, 1H), 7.38 (s, 1H), 7.24 (d, J=3.7 Hz, 1H), 7.14 (dd, J=1.3, 8.2 Hz, 1H), 6.60 (d, J=3.1 Hz, 1H), 5.07-4.98 (m, 1H), 4.52 (t, J=7.0 Hz, 1H), 3.94 (d, J=6.4 Hz, 1H), 2.92-2.71 (m, 2H), 2.29 (s, 3H), 2.12-2.04 (m, 1H), 1.99-1.80 (m, 3H), 1.25 (s, 3H).

### Example 7

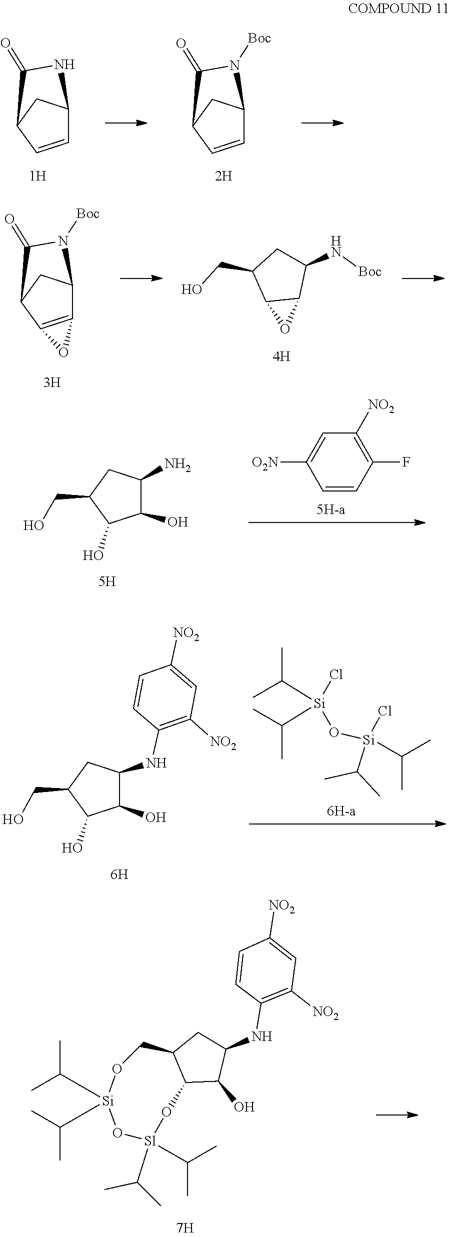

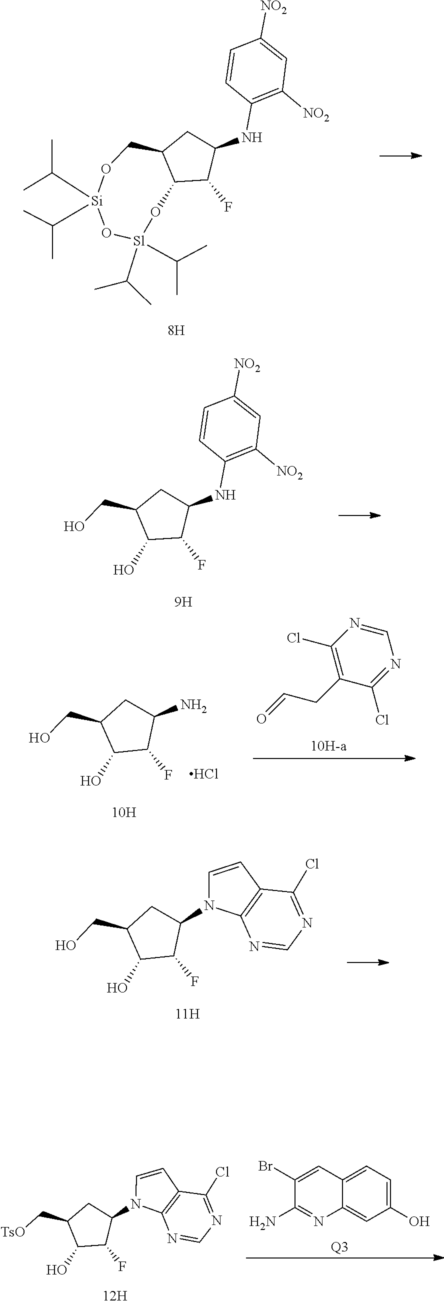

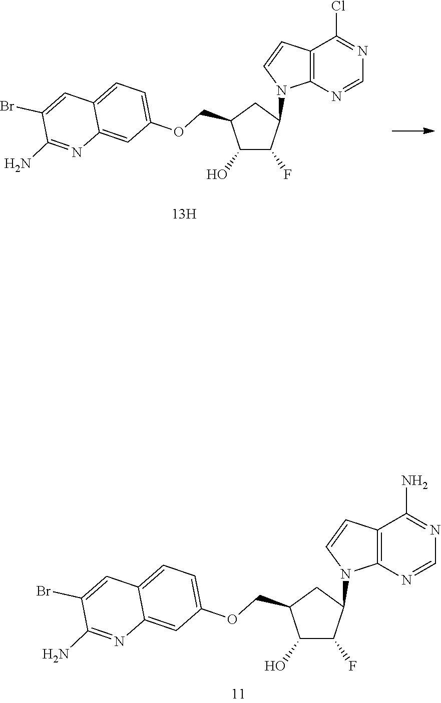 [SMILES: Nc1nc2cc(OC[C@H]3C[C@@H](n4ccc5c(Cl)ncnc54)[C@H](F)[C@@H]3O)ccc2cc1Br ; Nc1nc2cc(OC[C@H]3C[C@@H](n4ccc5c(N)ncnc54)[C@H](F)[C@@H]3O)ccc2cc1Br]

Tert-butoxycarbonyl tert-butyl carbonate (30.00 g, 137.46 mmol, 31.58 mL, 1.5 eq.) was added dropwise to a mixture of (1S,4R)-3-azabicyclo[2.2.1]hept-5-en-2-one (1H) (10 g, 91.64 mmol, 1 eq.), DMAP (1.12 g, 9.16 mmol, 0.1 eq.) and TEA (18.55 g, 183.27 mmol, 25.51 mL, 2 eq.) in DCM (150 mL) at 0° C. The mixture was stirred at 25° C. for 12 h. The reaction progress was monitored by TLC (PE:EA=3:1). Upon completion, the reaction was quenched by NaHCO₃ (sat. aq., 20 mL) and extracted with DCM (2×20 mL). The combined organic layers were washed with brine (2×20 mL), dried over MgSO₄ and concentrated to give a residue. The residue was purified by column chromatography (SiO₂, PE:EA=15:1 to 5:1) to afford tert-butyl (1S, 4R)-2-oxo-3-azabicyclo[2.2.1]hept-5-ene-3-carboxylate (2H) (16.5 g, 78.86 mmol, 86% yield) as a white solid. LCMS: (ESI): m/z calcd. for C₁₁H₁₅NNaO₃ 232.09 [M+Na]⁺, found 232.0.

m-CPBA (19.41 g, 95.58 mmol, 85% purity, 4 eq.) was added to a solution of 2H (5 g, 23.90 mmol, 1 eq.) in DCE (150 mL). The mixture was stirred at 25° C. for 96 h. The mixture was stirred at 25° C. for 12 h. The reaction progress was monitored by TLC (PE:EA=3:1). Upon completion, the reaction was quenched by Na₂S₂O₃ (sat. aq., 10 mL) and extracted with DCM (2×30 mL). The combined organic layers were washed with brine (2×30 mL), dried over MgSO₄ and concentrated to give a residue. The residue was purified by column chromatography (SiO₂, PE:EA=30:1 to 5:1) to afford tert-butyl (4S, 5R, 6R, 7S)-8-oxo-14-oxa-11-azatricyclooctane-11-carboxylate (3H) (4.6 g, 14.30 mmol, 60% yield) as a white solid. LCMS: (ESI): m/z calcd. for C₁₁H₁₅NNaO₄ 248.09 [M+Na]⁺, found 247.9.

NaBH₄ (2.65 g, 69.93 mmol, 5 eq.) was added in portions to a solution of 3H (4.5 g, 13.99 mmol, 1 eq.) in MeOH (120 mL) at 0° C. The mixture was stirred at 0° C. for 1 h. The reaction progress was monitored by TLC (PE:EA=1:1). Upon completion, the mixture was neutralized by a solution of 10% acetic acid in MeOH, and then extracted with DCM (3×50 mL). The combined organic layers were washed with brine (2×50 mL), dried over Na₂SO₄, and concentrated to give a residue. The residue was purified by column chromatography (SiO₂, PE:EA=15:1 to 3:1) to afford tert-butyl N-[(1S, 2R, 4R, 5R)-4-(hydroxymethyl)-6-oxabicyclo[3.1.0]hexan-2-yl]carbamate (4H) (2.35 g, 10.25 mmol, 71% yield) as a white solid. ¹H NMR (400 MHz, CDCl₃) δ: 5.79 (br d, J=7.5 Hz, 1H), 4.29 (br s, 1H), 3.93 (br d, J=10.3 Hz, 1H), 3.69 (td, J=3.5, 10.1 Hz, 1H), 3.44 (s, 2H), 2.42 (br d, J=9.0 Hz, 1H), 2.19-2.06 (m, 1H), 1.78 (br s, 1H), 1.46 (s, 9H), 1.43 (br s, 1H).

To a solution of NaOH (1 M, 88 mL, 9.17 eq.) in H₂O was added 4H (2.2 g, 9.60 mmol, 1 eq.). The mixture was stirred at 75° C. for 2 h. The mixture was stirred at 0° C. for 1 h. The reaction progress was monitored by TLC (DCM:MeOH=20:1). Upon completion, the reaction mixture was quenched by H-form ion-exchange resin (pH to ˜8) and then filtered. The filtrate was concentrated under reduced pressure to give a residue to afford (1R,2R,3R,5R)-3-amino-5-(hydroxymethyl)cyclopentane-1,2-diol (5H) (2.13 g, crude) as a yellow solid.

1-fluoro-2,4-dinitro-benzene (5H-a) (1.61 g, 8.66 mmol, 1.09 mL, 1 eq.) and Na₂CO₃ (917.36 mg, 8.66 mmol, 1 eq.) were added to a solution of 5H (1.93 g, 8.66 mmol, 1 eq., crude) in DMF (20 mL). The mixture was stirred at 25° C. for 2 h. The reaction progress was monitored by TLC (DCM:MeOH=20:1). Upon completion, the mixture was extracted with DCM (2×30 mL). The combined organic layers were washed with brine (2×30 mL), dried over Na₂SO₄, and concentrated to give a residue. The residue was purified by column chromatography (SiO₂, DCM:MeOH=100:1 to 20:1) to afford (1R,2R,3R,5R)-3-(2,4-dinitroanilino)-5-(hydroxymethyl)cyclopentane-1,2-diol (6H) (1.3 g, 4.07 mmol, 47% yield) as a yellow solid. LCMS: (ESI): m/z calcd. for C₁₂H₁₆N₃O₇ 314.09 [M+H]⁺, found 313.9.

Chloro-[chloro(diisopropyl)silyl]oxy-diisopropyl-silane (6H-a) (1.44 g, 4.56 mmol, 1.46 mL, 1.1 eq.) was added dropwise to a solution of 6H (1.3 g, 4.15 mmol, 1 eq.) in pyridine (15 mL). The mixture was stirred at 25° C. for 12 h. The reaction progress was monitored by TLC (PE:EA=3:1). Upon completion, the mixture was concentrated to give a residue. The residue was purified by column chromatography (SiO₂, PE:EA=20:1 to 5:1) to afford (6aR,8R,9R,9aR)-8-(2,4-dinitroanilino)-2,2,4,4-tetraisopropyl-6,6a,7,8,9,9a-hexahydrocyclopenta[f][1,3,5,2,4]trioxadisilocin-9-ol (7H) (1.62 g, 2.91 mmol, 70% yield) as a yellow solid. LCMS: (ESI): m/z calcd. for C₂₄H₄₂N₃O₈Si₂ 556.24 [M+H]⁺, found 556.1.

DAST (555.89 mg, 3.10 mmol, 455.65 μL, 90% purity, 1.5 eq.) was added to a solution of 7H (1.15 g, 2.07 mmol, 1 eq.) in DCM (25 mL) at 0° C. The mixture was stirred at 0° C. for 1 h. The reaction progress was monitored by TLC (PE:EA=3:1). Upon completion, the mixture was filtered and concentrated under reduced pressure to give a residue. The residue was purified by column chromatography (SiO₂, PE:EA=40:1 to 15:1) to afford (6aR,8R,9S,9aR)—N-(2,4-dinitrophenyl)-9-fluoro-2,2,4,4-tetraisopropyl-6,6a,7,8,9,9a-hexahydrocyclopenta[f][1,3,5,2,4]trioxadisilocin-8-amine (8H) (630 mg, 508.28 μmol, 24% yield) as a yellow solid. LCMS: (ESI): m/z calcd. for C₂₄H₄₁FN₃O₇Si₂ 558.24 [M+H]⁺, found 558.3.

NH₄F (470.63 mg, 12.71 mmol, 25 eq.) was added to a solution of 8H (630 mg, 508.28 μmol, 1 eq.) in MeOH (23 mL). The mixture was stirred at 25° C. for 12 h. The reaction progress was monitored by TLC (DCM:MeOH=20:1). Upon completion, the mixture was filtered and concentrated under reduced pressure to give a residue. The residue was purified by column chromatography (SiO₂, DCM:MeOH=200:1 to 100:1) to afford (1R, 2S, 3R, 5R)-3-(2,4-dinitroanilino)-2-fluoro-5-(hydroxymethyl) cyclopentanol (9H) (135 mg, 398.25 μmol, 78% yield) as a yellow solid. LCMS: (ESI): m/z calcd. for C₁₂H₁₅FN₃O₆ 316.09 [M+H]⁺, found 315.9.

Amberlite IRA 400(OH—) resin (2.2 g) was added to a solution of 9H (135 mg, 428.23 μmol, 1 eq.) in acetone (3.8 mL) and H₂O (1.9 mL). The mixture was stirred at 25° C. for 12 h. The reaction progress was monitored by TLC (DCM:MeOH=10:1). Upon completion, the mixture was filtered and washed with water (2 mL). HCl (1 N, 0.8 mL) was added, and the solution was washed with EA (3×5 mL). The mixture was concentrated under reduced pressure to give (1R, 2S, 3R, 5R)-3-amino-2-fluoro-5 (hydroxymethyl) cyclopentanol (10H) (97 mg, crude, HCl salt) as a pale yellow solid. ¹H NMR (400 MHz, DMSO-d₆) δ: 8.59 (br s, 3H), 4.91-4.68 (m, 1H), 3.61-3.49 (m, 1H), 3.49-3.30 (m, 2H), 2.15 (td, J=8.4, 13.2 Hz, 1H), 1.95 (br d, J=5.3 Hz, 1H), 1.33 (td, J=9.3, 13.2 Hz, 1H).

Et₃N (181.53 mg, 1.79 mmol, 249.70 μL, 3 eq.) and 2-(4, 6-dichloropyrimidin-5-yl) acetaldehyde (10H-a) (114.22 mg, 597.98 μmol, 1 eq.) was added to a solution of 10H (185 mg, 597.98 μmol, 1 eq., HCl) in EtOH (3 mL). The mixture was stirred at 80° C. for 2 h. The reaction progress was monitored by TLC (DCM:MeOH=10:1). Upon completion, the mixture was filtered and concentrated under reduced pressure to give a residue. The residue was purified by column chromatography (SiO₂, DCM:MeOH=200:1 to 50:1) to afford (1R, 2S, 3R, 5R)-3-(4-chloropyrrolo[2, 3-d] pyrimidin-7-yl)-2-fluoro-5-(hydroxymethyl) cyclopentanol (11H) (144 mg, 504.02 μmol, 84% yield) as a yellow oil. LCMS: (ESI): m/z calcd. for C₁₂H₁₄ClFN₃O₂ 286.07 [M+H]⁺, found 285.9.

4-methylbenzenesulfonyl chloride (80.08 mg, 420.02 μmol, 1.2 eq.) was added to a solution of 11H (100 mg, 350.02 μmol, 1 eq.) in pyridine (0.5 mL) at 0° C. The mixture was stirred at 25° C. for 2 h. The reaction progress was monitored by TLC (DCM:MeOH=20:1). The mixture was filtered and concentrated under reduced pressure to give a residue. The residue was purified by column chromatography (SiO₂, PE:EA=20:1 to 1:2) to afford [(1R,2R,3S,4R)-4-(4-chloropyrrolo[2,3-d]pyrimidin-7-yl)-3-fluoro-2-hydroxy-cyclopentyl]methyl 4-methylbenzenesulfonate (12H) (57 mg, 129.58 μmol, 37% yield) as a yellow solid. LCMS: (ESI): m/z calcd. for C₁₉H₂₀ClFN₃O₄S 440.08 [M+H]⁺, found 440.0.

Cs₂CO₃ (148.88 mg, 456.94 μmol, 3 eq.) was added to a mixture of 12H (67 mg, 152.31 μmol, 1 eq.) and 2-amino-3-bromo-quinolin-7-ol (36.41 mg, 152.31 μmol, 1 eq.) (Q3) in DMF (0.5 mL). The mixture was stirred at 25° C. for 15 h. The reaction progress was monitored by TLC (DCM:MeOH=20:1). Upon completion, the mixture was diluted with water (5 mL) and extracted with EA (3×3 mL). The combined organic layers were washed with brine (2×5 mL), dried over Na₂SO₄, and concentrated to give a residue. The residue was purified by prep-TLC (SiO₂, DCM:MeOH=20:1) to afford (1R, 2S, 3R, 5R)-5-[(2-amino-3-bromo-7-quinolyl) oxymethyl]-3-(4-chloropyrrolo[2,3-d] pyrimidin-7-yl)-2-fluoro-cyclopentanol (13H) (41 mg, 79.94 μmol, 52% yield) as a white solid. LCMS: (ESI): m/z calcd. for C₂₁H₁₉BrClFN₅O₂ 508.03 [M+H]⁺, found 508.0.

NH₃.H₂O (4.55 g, 36.35 mmol, 5 mL, 28% purity, 526.34 eq.) was added to a solution of 13H (35 mg, 69.07 μmol, 1 eq.) in dioxane (5 mL). The mixture was stirred at 100° C. for 48 h. The reaction progress was monitored by TLC (DCM:MeOH=10:1). Upon completion, the mixture was filtered and concentrated under reduced pressure to give a residue. The residue was purified by prep-HPLC (column: Phenomenex Gemini-NX 150*30 mm*5 um; mobile phase: [water (0.04% NH₃H₂O+10 mM NH₄HCO₃)-ACN]; B %: 26%-56%, 8 min) to afford (1R,2S,3R,5R)-5-[(2-amino-3-bromo-7-quinolyl)oxymethyl]-3-(4-aminopyrrolo[2,3-d]pyrimidin-7-yl)-2-fluoro-cyclopentanol (11) (12 mg, 24.25 μmol, 35% yield) as a white solid. LCMS: (ESI): m/z calcd. for C₂₁H₂₁BrFN₆O₂ 487.08 [M+H]⁺, found 487.1. ¹H NMR (400 MHz, CD₃OD) δ: 8.21 (s, 1H), 8.10 (s, 1H), 7.55 (d, J=9.0 Hz, 1H), 7.31 (d, J=3.5 Hz, 1H), 7.04 (d, J=2.3 Hz, 1H), 7.00 (d, J=8.8 Hz, 1H), 6.66 (d, J=3.5 Hz, 1H), 5.37-5.11 (m, 2H), 4.40 (td, J=5.6, 13.2 Hz, 1H), 4.28 (br dd, J=4.6, 8.4 Hz, 2H), 2.62 (br d, J=7.8 Hz, 2H), 2.08-1.96 (m, 1H).

### Example 8

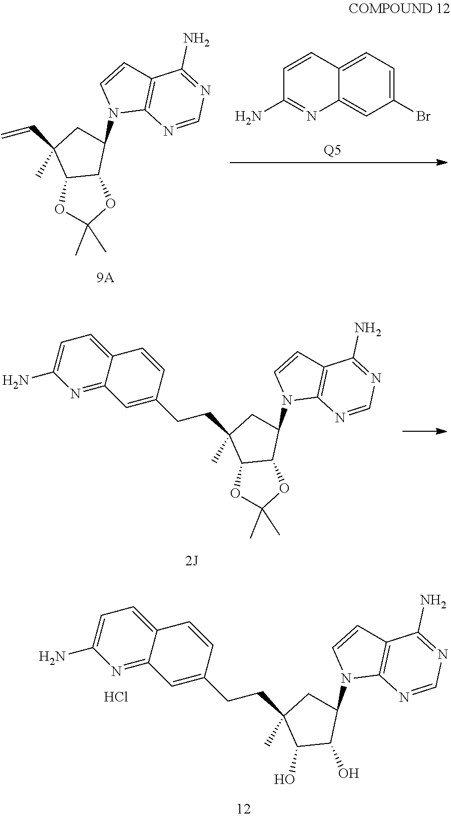

To a solution of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (180 mg, 572.6 μmol, 1 eq.) in THF (6 mL) was added 9-BBN dimer (304.9 mg, 1.3 mmol, 2.2 eq.). The mixture was stirred at 50° C. for 2 h and cooled to 20° C. K₃PO₄ (607.7 mg, 2.9 mmol, 5 eq.) and H₂O (0.6 mL) were added. The mixture was stirred at 20° C. for 0.5 h. Then 7-bromoquinolin-2-amine (153.3 mg, 687.1 μmol, 1.2 eq.) and Pd(dppf)Cl₂ (41.9 mg, 57.3 μmol, 0.1 eq.) were added. The mixture was stirred at 70° C. for 12 h. The reaction progress was monitored by LCMS. Upon completion, the mixture was diluted with brine (10 mL). The mixture was extracted with EA (3×20 mL). The organic layers were combined, dried over anhydrous Na₂SO₄, and concentrated to afford a residue. The residue was purified by reversed-phase HPLC (0.1% NH₃.H₂O) to afford 7-[2-[(3aR,4S,6R,6aS)-6-(4-aminopyrrolo[2,3-d]pyrimidin-7-yl)-2,2,4-trimethyl-3a,5,6,6a-tetrahydrocyclopenta[d][1,3]dioxol-4-yl]ethyl]quinolin-2-amine (2J) (175 mg, 64.5% yield) as white solid. LCMS: (ESI): m/z calcd. for C₂₆H₃₁N₆O₂ 459.25 [M+H]⁺, found 459.4.

A mixture of 2J (230 mg, 501.58 μmol, 1 eq.), HCl (4 M, 3 mL) and THF (6 mL) was stirred at rt for 6 h. The reaction progress was monitored by LCMS. Upon completion, the mixture was concentrated under reduced pressure to afford a solid. The solid was washed with MeCN:H₂O (10:1.6 mL) to afford (1S,2R,3S,5R)-5-(4-amino-7H-pyrrolo[2,3-d]pyrimidin-7-yl)-3-(2-(2-aminoquinolin-7-yl)ethyl)-3-methylcyclopentane-1,2-diol (12) as a hydrochloride salt (white solid, 180 mg, 72% yield). LCMS: (ESI): m/z calcd. for C₂₃H₂₇N₆O₂ 419.22 [M+H]⁺, found 419.3. ¹H NMR (CD₃OD) δ: 8.31 (d, J=9.3 Hz, 1H), 8.25 (s, 1H), 7.82 (d, J=8.2 Hz, 1H), 7.58 (d, J=3.7 Hz, 1H), 7.53 (s, 1H), 7.45 (dd, J=1.3, 8.2 Hz, 1H), 7.01 (d, J=9.5 Hz, 1H), 6.92 (d, J=3.5 Hz, 1H), 5.19-5.07 (m, 1H), 4.61-4.49 (m, 1H), 3.96 (d, J=6.4 Hz, 1H), 3.02-2.78 (m, 2H), 2.17-1.80 (m, 4H), 1.25 (s, 3H).

### Example 9

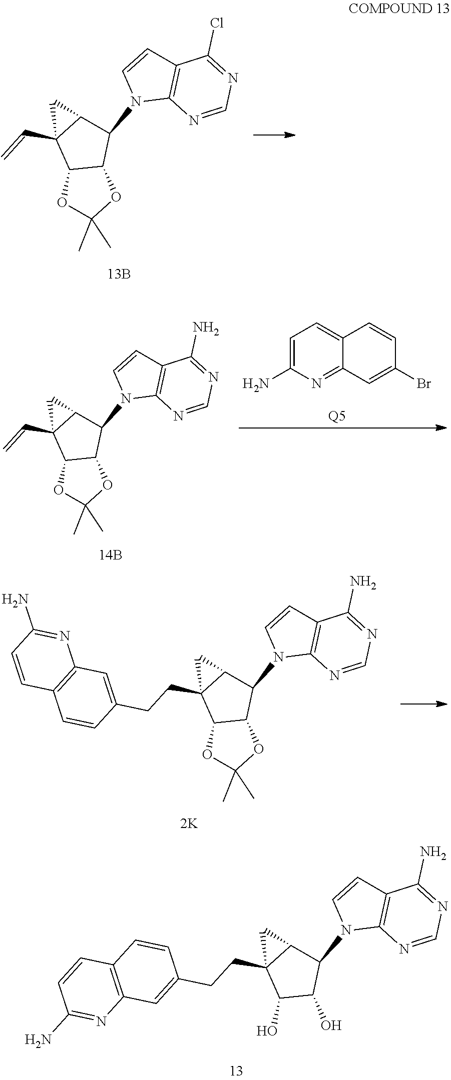

NH₃.H₂O (20 mL, 28% wt) was added to a solution of 4-chloro-7-[(11S,12R,13S,14R,16S)-15,15-dimethyl-16-vinyl-20,21-dioxatricyclononan-12-yl]pyrrolo[2,3-d]pyrimidine (13B) (788 mg, 2.37 mmol, 1 eq.) in dioxane (20 mL). The mixture was stirred at 100° C. for 48 h. The reaction progress was monitored by TLC (DCM:MeOH=20:1). Upon completion, the mixture was filtered and concentrated under reduced pressure to give a residue. The residue was purified by flash silica gel chromatography (ISCO®; 12 g Sepa Flash® Silica Flash Column, Eluent of 0˜8% DCM/MeOH @ 30 mL/min) to afford 7-[(11 S,12R,13S,14R,16S)-15,15-dimethyl-16-vinyl-21,22-dioxatricyclononan-12-yl]pyrrolo[2,3-d]pyrimidin-4-amine (14B) (773 mg, 2.03 mmol, 85% yield) as a white solid. LCMS: (ESI): m/z calcd. for C₁₇H₂₁N₄O₂ 313.16 [M+H]⁺, found 312.9.

9-BBN dimer (1.05 g, 4.32 mmol, 2.7 eq.) was added to a solution of 14B (500 mg, 1.60 mmol, 1 eq.) in THF (10 mL), and the mixture was stirred at 50° C. for 3.5 h under Ar atmosphere. The mixture was cooled to rt, and then K₃PO₄ (1.70 g, 8.00 mmol, 5 eq.) in H₂O (1 mL) were added. The resulting mixture was stirred at 25° C. for 0.5 h, and then 7-bromoquinolin-2-amine (Q5) (357.06 mg, 1.60 mmol, 1 eq.) and Pd(dppf)Cl₂ (117.12 mg, 160.07 μmol, 0.1 eq.) were added. The mixture was purged with Ar (3×), and then stirred at 72° C. for 12 h. The reaction progress was monitored by TLC (DCM:MeOH=10:1). Upon completion, the mixture was extracted with EA (3×10 mL). The combined organic layers were washed with brine (3×20 mL), dried over Na₂SO₄ and concentrated to give a residue. The residue was purified by prep-HPLC Combi-flash reversed-phase C-18 column chromatography (10%˜60% CH₃CN/water (1 mL/3 L NH₃.H₂O in water)@ 75 mL/min) to afford 7-[2-[(20S,21R,22S,23R,25R)-21-(4-aminopyrrolo[2,3-d]pyrimidin-7-yl)-24,24-dimethyl-32,33-dioxatricyclononan-25-yl]ethyl]quinolin-2-amine (2K) (446 mg, 976.92 μmol, 19% yield) as a yellow solid. LCMS: (ESI): m/z calcd. for C₂₆H₂₉N₆O₂ 457.23 [M+H]⁺, found 457.1.

To a solution of 2K (446 mg, 976.92 μmol, 1 eq.) in THF (10 mL) was added HCl (4 M, 5 mL). The mixture was stirred at 25° C. for 12 h. The reaction progress was monitored by TLC (DCM:MeOH=5:1). Upon completion, the mixture was filtered and concentrated under reduced pressure to give a residue (400 mg, crude, HCl salt) as a brown solid. This residue (200 mg) was purified by prep-HPLC (column: Phenomenex Gemini-NX 150*30 mm*5 um; mobile phase: [water (0.04% NH₃H₂O+10 mM NH₄HCO₃)-ACN]; B %: 20%-50%, 8 min) to afford (1R,2R,3S,4R,5S)-4-(4-aminopyrrolo[2,3-d]pyrimidin-7-yl)-1-[2-(2-amino-7-quinolyl)ethyl]bicyclo[3.1.0]hexane-2,3-diol (13) (115 mg, 274.39 μmol, 58% yield) as a white solid. LCMS: (ESI): m/z calcd. for C₂₃H₂₅N₆O₂ 417.2 [M+H]⁺, found 417.2. ¹H NMR (400 MHz, CD₃OD) δ: 8.09 (s, 1H), 7.90 (d, J=8.8 Hz, 1H), 7.57 (d, J=8.3 Hz, 1H), 7.41 (s, 1H), 7.18 (dd, J=1.4, 8.2 Hz, 1H), 7.01 (d, J=3.8 Hz, 1H), 6.77 (d, J=9.0 Hz, 1H), 6.57 (d, J=3.8 Hz, 1H), 4.95 (s, 1H), 4.60 (br d, J=6.5 Hz, 1H), 3.86 (d, J=6.8 Hz, 1H), 3.12-2.87 (m, 2H), 2.30-2.18 (m, 1H), 2.05-1.93 (m, 1H), 1.47 (t, J=4.3 Hz, 1H), 1.39 (dd, J=3.3, 8.5 Hz, 1H), 0.68 (dd, J=5.3, 7.0 Hz, 1H).

### Example 10

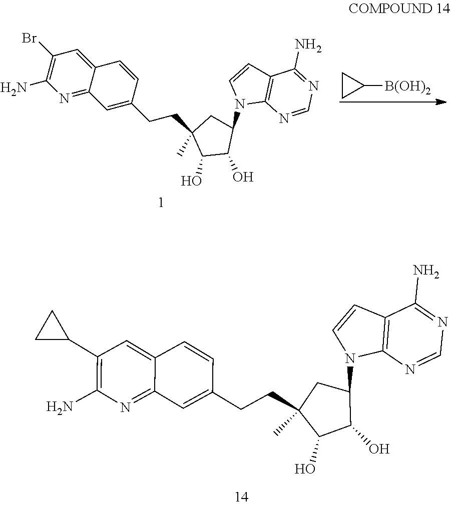

To a solution of 1 (40 mg, 80.4 μmol, 1 eq.) in dioxane (1 mL) and H₂O (0.2 mL) were added K₂CO₃ (27.8 mg, 201.05 μmol, 2.5 eq.), cyclopropyl boronic acid (17.3 mg, 201.1 μmol, 2.5 eq.), and Pd(dppf)Cl₂ (5.9 mg, 8.0 μmol, 0.1 eq.). The mixture was stirred at 100° C. for 16 h under Ar atmosphere. The reaction was monitored by LC-MS. Upon completion, the mixture was filtrated. The filter cake was washed with dioxane/water (5:1) (4 mL). The residue was purified by prep-HPLC (HCl condition; water (0.05% HCl)-ACN, column: Phenomenex Gemini-NX 150*30 mm*5 um, begin: 10, end: 30, Gradient Time (min): 7 min, 100% B Hold Time (min): 0, Flow Rate (mL/min): 35) to give (1S,2R,3S,5R)-3-[2-(2-amino-3-cyclopropyl-7-quinolyl)ethyl]-5-(4-aminopyrrolo[2,3-d]pyrimidin-7-yl)-3-methylcyclopentane-1,2-diol (14) (24 mg, 45.05 μmol, 56.0% yield, 99.8% purity, 2 HCl) as a white solid. LCMS: (ESI): m/z calcd. for C₂₆H₃₁N₆O₂ 459.24 [M+H]⁺, found 459.2. ¹HNMR (400 MHz, CD₃OD) δ: 8.25 (s, 1H), 8.12 (s, 1H), 7.79 (d, J=8.3 Hz, 1H), 7.56 (d, J=3.4 Hz, 1H), 7.53 (s, 1H), 7.40-7.44 (m, 1H), 6.90-6.93 (m, 1H), 5.08-5.16 (m, 1H), 4.52-4.58 (m, 1H), 3.96 (d, J=6.4 Hz, 1H), 2.81-3.00 (m, 2H), 1.97-2.14 (m, 2H), 1.81-1.96 (m, 3H), 1.25 (s, 3H), 1.11-1.17 (m, 2H), 0.80-0.85 (m, 2H).

### Example 11

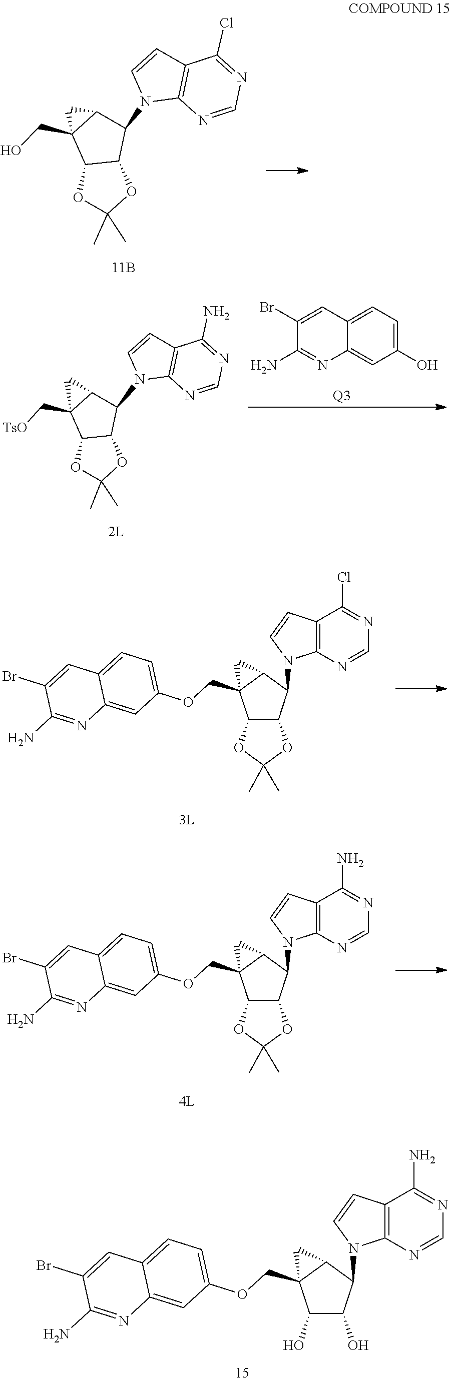

To a solution of [(10S,11R,12S,13R,15R)-11-(4-chloropyrrolo[2,3-d]pyrimidin-7-yl)-14,14-dimethyl-20,21-dioxatricyclononan-15-yl]methanol (11B) (500 mg, 1.49 mmol, 1 eq.) in DCM (10 mL) were added Et₃N (1.51 g, 14.90 mmol, 2.1 mL, 10 eq.), DMAP (36.4 mg, 298.0 μmol, 0.2 eq.), and 4-methylbenzenesulfonyl chloride (852.2 mg, 4.47 mmol, 3 eq.). The mixture was stirred at 25° C. for 16 h. The mixture was concentrated under reduced pressure to give a residue. The residue was purified by flash silica gel chromatography (ISCO®; 12 g SepaFlash® Silica Flash Column, Eluent of 0˜32% EA:PE gradient @ 36 mL/min) to give 2 L (420 mg, 831.5 μmol, 55.8% yield) as a white solid. LCMS: (ESI): m/z calcd. for C₂₃H₂₅ClN₃O₅S 490.11 [M+H]⁺, found 490.1.

To a mixture of 2L (345 mg, 704.12 μmol, 1 eq.) and 2-amino-3-bromo-quinolin-7-ol (Q3) (168.33 mg, 704.12 μmol, TO eq.) in DMF (3 mL) was added Cs₂CO₃ (688.25 mg, 2.11 mmol, 3.0 eq.). The mixture was stirred at 20° C. for 16 h. The reaction progress was monitored by TLC. The mixture was then diluted with H₂O (30 mL) and extracted with EtOAc (3×10 mL). The combined organic layers were washed with brine (2×20 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜80% EA:PE gradient @ 30 mL/min). 3-bromo-7-[[(19S,20R,21S,22R,24R)-20-(4-chloropyrrolo[2,3-d]pyrimidin-7-yl)-23,23-dimethyl-30,31-dioxatricyclononan-24-yl]methoxy]quinolin-2-amine (3L) (280 mg, 482.7 μmol, 68% yield, 96% purity) was obtained as a light yellow solid. LCMS: (ESI): m/z calcd. for C₂₅H₂₅BrClN₅O₃ 558.07 [M+H]⁺, found 557.9.

A mixture of 3L (280 mg, 502.84 μmol, 1 eq.) and NH₃.H₂O (5 mL, 25% wt) in dioxane (5 mL) was heated in a sealed tube at 100° C. for 48 h. The reaction progress was monitored by LCMS. The mixture was then extracted with DCM (3×10 mL). The combined organic phase was washed with brine (20 mL), dried over Na₂SO₄, filtered and concentrated to give a residue. The residue was purified by prep-HPLC (column: Phenomenex Gemini-NX 150*30 mm*5 μm; mobile phase: [water (0.04% NH₃H₂O+10 mM NH₄HCO₃)-ACN]; B %: 42%-72%, 8 min). 7-[[(19S,20R,21S,22R,24R)-20-(4-aminopyrrolo[2,3-d]pyrimidin-7-yl)-23,23-dimethyl-31,32-dioxatricyclononan-24-yl]methoxy]-3-bromo-quinolin-2-amine (4L) (140 mg, 247.48 μmol, 49.22% yield, 95% purity) was obtained as alight yellow solid. LCMS: (ESI): m/z calcd. for C₂₅H₂₆BrN₆O₃ 539.12 [M+H]⁺, found 539.2.

To a mixture of 4L (140 mg, 260.5 μmol, 1.0 eq.) in THF (6 mL) was added HCl (4 M, 2.6 mL). The mixture was then stirred at 20° C. for 16 h. The reaction progress was monitored by LCMS. Upon completion the solution was adjusted to pH=8 with NH₃.H₂O and concentrated under reduced pressure to give a residue. The residue was purified by prep-HPLC (column: Phenomenex Gemini-NX 150*30 mm*5 μm; mobile phase: [water (0.04% NH₃H₂O+10 mM NH₄HCO₃)-ACN]; B %: 25%-55%, 8 min). (1R,2R,3S,4R,5S)-1-[(2-amino-3-bromo-7-quinolyl)oxymethyl]-4-(4-aminopyrrolo[2,3-d]pyrimidin-7-yl)bicyclo[3.1.0]hexane-2,3-diol (15) (74 mg, 146.2 μmol, 56% yield) was obtained as a white solid. LCMS: (ESI): m/z calcd. for C₂₂H₂₂BrN₆O₃ 497.09 [M+H]⁺, found 497.1. ¹H NMR (400 MHz, CD₃OD) δ: 8.22 (s, 1H), 8.11 (s, 1H), 7.71 (d, J=3.7 Hz, 1H), 7.59 (d, J=8.8 Hz, 1H), 7.08-7.01 (m, 2H), 6.66 (d, J=3.4 Hz, 1H), 5.10 (s, 1H), 4.84-4.79 (m, 1H), 4.61 (s, 1H), 3.87 (d, J=10.3 Hz, 1H), 3.78 (d, J=6.8 Hz, 1H), 1.77-1.71 (m, 2H), 0.92 (br s, 1H).

### Example 12

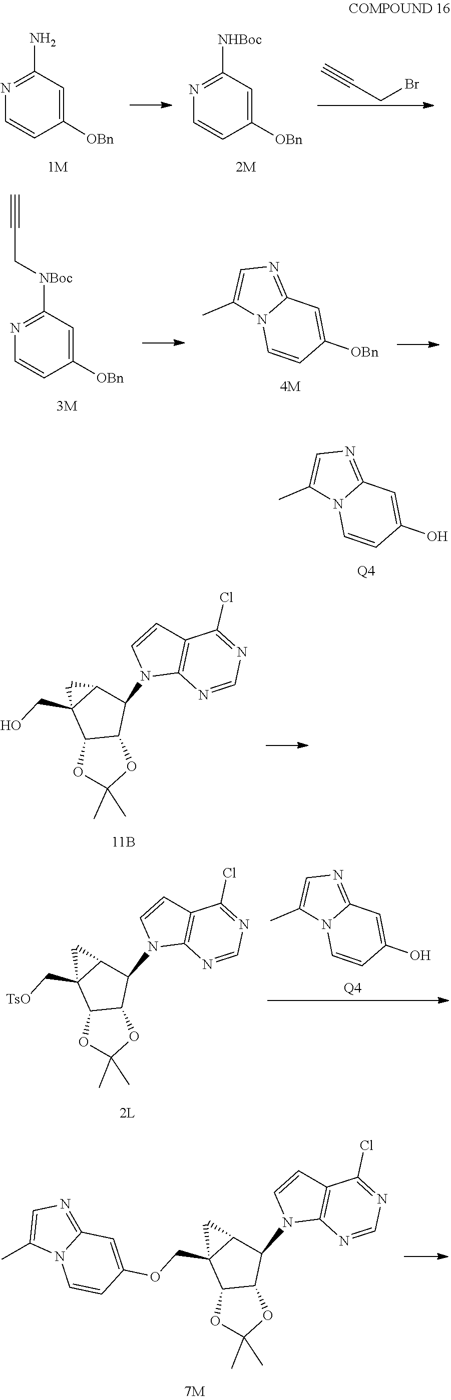

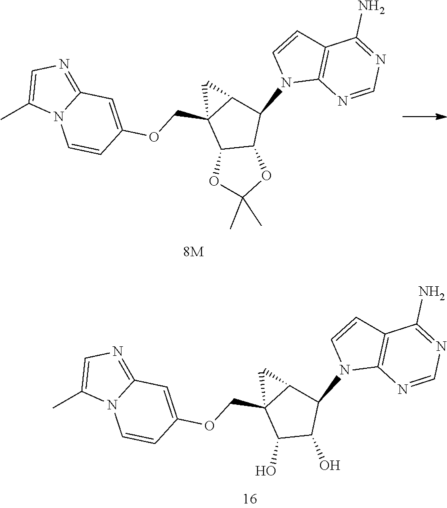 [SMILES: Cc1cnc2cc(OC[C@@]34C[C@@H]3[C@@H](n3ccc5c(N)ncnc53)[C@@H]3OC(C)(C)O[C@@H]34)ccn12 ; Cc1cnc2cc(OC[C@@]34C[C@@H]3[C@@H](n3ccc5c(N)ncnc53)[C@H](O)[C@@H]4O)ccn12]

A mixture of 4-benzyloxypyridin-2-amine (11B) (100 mg, 499.4 μmol, 1 eq.) and Boc₂O (119.9 mg, 549.4 μmol, 1.1 eq.) in t-BuOH (1.5 mL) was stirred at 50° C. for 1 h. The reaction progress was monitored by LCMS. Upon completion, the mixture was cooled to rt and diluted with EtOH (5 mL). The precipitate was filtered and dried under high vacuum to give crude 2L (116 mg, 363.1 μmol, 72.7% yield) as a white solid. LCMS: (ESI): m/z calcd. for C₁₇H₂₁N₂O₃ 301.15 [M+H]⁺, found 301.0.

To a solution of 2L (116 mg, 386.2 μmol, 1 eq.) in DMF (2 mL) was added NaH (23.2 mg, 579.3 μmol, 60% purity, 1.5 eq.) in portions at 17° C. under a N₂ atmosphere. Upon completion of the addition, the mixture was stirred for 10 min. 3-bromoprop-1-yne (68.9 mg, 579.3 μmol, 49.9 μL, 1.5 eq.) was then added, and the mixture was stirred at 17° C. for 1 h. The reaction progress was monitored by LCMS. Upon completion, the reaction was quenched by addition H₂O (3 mL) and extracted with EA (2×3 mL). The combined organic layers were washed with H₂O (2×3 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜22% EA:PE gradient @ 36 mL/min) to give 3M (110 mg, 312.1 μmol, 80.8% yield) as a light yellow oil. LCMS: (ESI): m/z calcd. for C₂₀H₂₃N₂O₃ 339.16 [M+H]⁺, found 339.0.

To a solution of 3M (110 mg, 325.1 μmol, 1 eq.) in THF (3 mL) was added t-BuOK (43.8 mg, 390.1 μmol, 1.2 eq.). The mixture was stirred at 15° C. for 30 min. The reaction progress was monitored by LCMS. Upon completion, the mixture was diluted with H₂O (3 mL) and extracted with EA (2×3 mL). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0-5% Methanol/Dichloromethane @ 36 mL/min) to give 4M (39 mg, 148.9 μmol, 45.8% yield) as a yellow solid. LCMS: (ESI): m/z calcd. for C₁₅H₁₅N₂O 239.11 [M+H]⁺, found 238.9.

To a solution of 4M (2.3 g, 9.65 mmol, 1 eq.) in MeOH (60 mL) was added Pd/C (10%, 1 g) under N₂ atmosphere. The mixture was stirred under H₂ (15 Psi) at 15° C. for 1 h. The reaction progress was monitored by LCMS. Upon completion, the mixture was filtered and concentrated under reduced pressure to give a crude Q4 (1.48 g, crude, 85% purity) as a black solid. LCMS: (ESI): m/z calcd. for C₈H₉N₂O 149.06 [M+H]⁺, found 149.2.

To a solution of [(10S,11R,12S,13R,15R)-11-(4-chloropyrrolo[2,3-d]pyrimidin-7-yl)-14,14-dimethyl-20,21-dioxatricyclononan-15-yl]methanol (11B) (500 mg, 1.49 mmol, 1 eq.) in DCM (10 mL) was added Et₃N (1.51 g, 14.90 mmol, 2.1 mL, 10 eq.), DMAP (36.4 mg, 298.0 μmol, 0.2 eq.) and 4-methylbenzenesulfonyl chloride (852.2 mg, 4.47 mmol, 3 eq.). The mixture was stirred at 25° C. for 16 h. The mixture was concentrated under reduced pressure to give a residue. The residue was purified by flash silica gel chromatography (ISCO®; 12 g SepaFlash® Silica Flash Column, Eluent of 0˜32% EA:PE gradient @ 36 mL/min) to give 2L (420 mg, 831.5 μmol, 55.8% yield) as a white solid. LCMS: (ESI): m/z calcd. for C₂₃H₂₅ClN₃O₅S 490.11 [M+H]⁺, found 490.1.

To a mixture of 2L (500 mg, 1.02 mmol, 1 eq.) and Q4 (181.4 mg, 1.22 mmol, 1.2 eq.) in DMF (5 mL) was added Cs₂CO₃ (997.5 mg, 3.06 mmol, 3 eq.). The mixture was stirred at 20° C. for 16 h. The reaction progress was monitored by LCMS. Upon completion, the mixture was diluted with H₂O (30 mL) and extracted with EA (3×10 mL). The combined organic layers were washed with brine (2×20 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜5% MeOH/DCM gradient @ 30 mL/min) to give 7M (350 mg, 721.14 μmol, 70.67% yield, 96% purity) as a slightly yellow solid. LCMS: (ESI): m/z calcd. for C₂₄H₂₅ClN₅O₃ 466.16 [M+H]⁺, found 466.3.

To a solution of NH₃.H₂O (10 mL, 25% wt) and dioxane (10 mL) was added 7M (350 mg, 751.2 μmol, 1 eq.). The mixture was heated in a sealed tube at 100° C. and stirred for 40 h. The mixture was extracted with EA (2×10 mL). The combined organic layers were dried over Na₂SO₄, filtered and concentrated under reduced pressure to give a residue. The residue was purified by prep-HPLC (basic condition: column: Phenomenex Gemini-NX 150*30 mm*5 μm; mobile phase: [water (0.04% NH₃.H₂O+10 mM NH₄HCO₃)-ACN]; B %: 28%-58%, 8 min) to give 8M (226 mg, 491 μmol, 65.4% yield) as a light yellow solid. LCMS: (ESI): m/z calcd. for C₂₄H₂₇N₆O₃ 447.21 [M+H]⁺, found 447.3.

To a solution of 8M (226 mg, 506.16 μmol, 1 eq.) in THF (6 mL) was added HCl (4 M, 3 mL, 23.71 eq.). The mixture was stirred at 25° C. for 12 h. The reaction progress was monitored by LCMS. Upon completion, the mixture was concentrated under reduced pressure to give a residue. The residue was diluted with H₂O (10 mL) and lyophilized to give 16 (2 HCl, 245 mg, 505.5 μmol, 99.9% yield) as a white solid. LCMS: (ESI): m/z calcd. for C₂₁H₂₃N₆O₃ 407.18 [M+H]⁺, found 407.2. ¹H NMR (400 MHz, CD₃OD) δ: 8.55 (d, J=7.5 Hz, 1H), 8.30 (s, 1H), 7.76 (d, J=3.8 Hz, 1H), 7.63 (d, J=1.0 Hz, 1H), 7.37-7.25 (m, 2H), 7.02 (d, J=3.8 Hz, 1H), 5.24 (s, 1H), 4.85-4.80 (m, 2H), 4.16 (d, J=10.3 Hz, 1H), 3.93 (d, J=6.5 Hz, 1H), 2.59 (d, J=1.0 Hz, 3H), 1.84-1.72 (m, 2H), 1.06-0.96 (m, 1H).

### Example 13

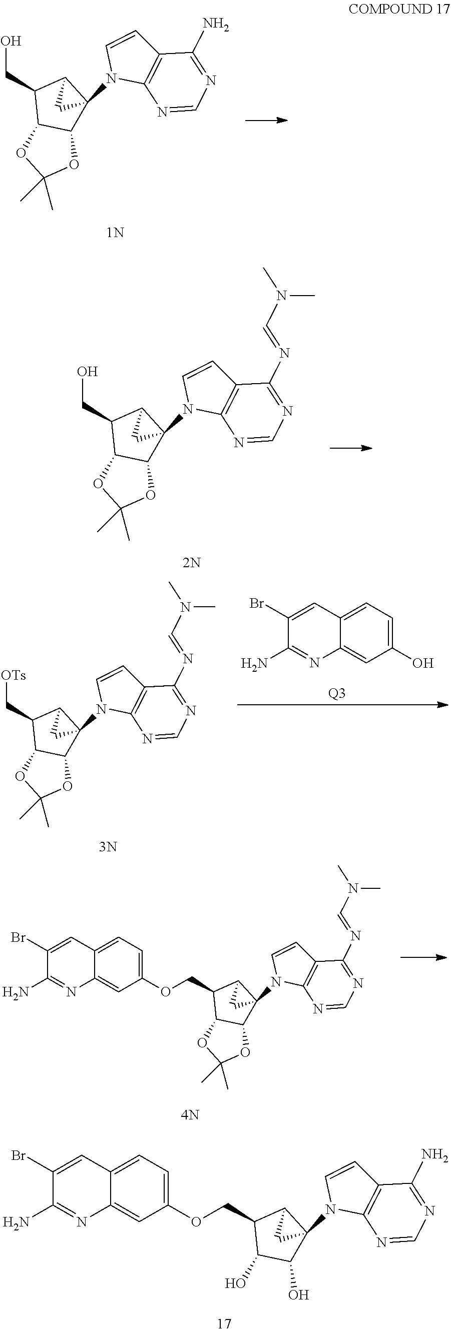

A mixture of Rac-[(10S,11R,12R,13S)-15-(4-aminopyrrolo[2,3-d]pyrimidin-7-yl)-14,14-dimethyl-21,22-dioxatricyclononan-11-yl]methanol (25 mg, 79.03 μmol, 1 eq.) in THF (2 mL) was added 1,1-dimethoxy-N,N-dimethyl-methanamine (188.4 mg, 1.58 mmol, 210.0 μL, 20 eq.), and the mixture was then stirred at 60° C. for 24 h. The reaction progress was monitored by LCMS. Upon completion, the mixture was concentrated to afford a residue. The residue was purified by pre-TLC (DCM:MeOH=10:1) to afford Compound rac-N′-[7-[(13S,14R,15R,16S)-14-(hydroxymethyl)-17,17-dimethyl-25,26-dioxatricyclononan-18-yl]pyrrolo[2,3 d]pyrimidin-11-yl]-N,N-dimethyl-formamidine (2N) (26 mg, 70.0 μmol, 88.6% yield) as a brown gum. LCMS: (ESI): m/z calcd, for C₁₉H₂₆N₅O₃ 372.20 [M+H]⁺, found 372.1.

4-methylbenzenesulfonyl chloride (40.1 mg, 210.0 μmol, 3 eq.) was added to the mixture of 2N (26 mg, 70.0 μmol, 1 eq.), TEA (35.4 mg, 350.0 μmol, 49 μL, 5 eq.) and DMAP (1.7 mg, 14.0 μmol, 0.2 eq.) in DCM (1 mL). After being stirred at 25° C. for 2 h, the mixture was concentrated and purified by pre-TLC (EA) to afford rac-[(20S,21R,22R,23S)-25-[4-[(E)-dimethylaminomethyleneamino]pyrrolo[2,3-d]pyrimidin-7-yl]-24,24-dimethyl-33,34-dioxatricyclononan-21-yl]methyl 15-methylbenzenesulfonate (3N) (15 mg, 28.5 μmol, 40.8% yield) as a brown gum. LCMS: (ESI): m/z calcd, for C₂₆H₃₂N₅O₅S 526.21 [M+H]⁺, found 526.1.

To a solution of 3N (15 mg, 28.54 μmol, 1 eq.) and 2-amino-3-bromo-quinolin-7-ol (6.8 mg, 28.5 μmol, 1 eq.) in DMF (1 mL) was added Cs₂CO₃ (27.9 mg, 85.6 μmol, 3 eq.). The mixture was stirred at 20° C. for 12 h. The reaction progress was monitored by LCMS. Upon completion, the mixture was concentrated to afford a residue. The residue was purified by pre-TLC (EA, 100%) to afford rac-N′-[7-[(22S,23R,24R,25S)-23-[(2-amino-3-bromo-7-quinolyl)oxy methyl]-26,26-dimethyl-35,36-dioxatricyclononan-27-yl]pyrrolo[2,3-d]pyrimidin-20-yl]-N,N-dimethyl-formamidine (4N) (7 mg, 8.1 μmol, 28.5% yield, 68.8% purity) was obtained as a white solid. LCMS: (ESI): m/z calcd, for C₂₈H₃₁BrN₇O₃ 594.16 [M+H]⁺, found 594.2.

To a solution of 4N (7 mg, 8.1 μmol, 68.8% purity, 1 eq.) in THF (0.6 mL) was added HCl (4 M, 0.3 mL). The mixture was stirred at 20° C. for 12 h. The reaction progress was monitored by LCMS. Upon completion, the mixture was concentrated under reduced pressure to give a residue. The residue was neutralized by NH₄OH (0.1 mL), and then purified by prep-HPLC (Column: Boston Prime C18 150*30 mm*5 um, Condition: water (0.04% NH₃H₂O+10 mM NH₄HCO₃)-ACN, begin B: 30%, end B: 60%, Gradient Time (min): 8, 100% B Hold Time (min): 2, Flow Rate (mL/min): 25) to give rac-(2S,3R,4R,5S)-4-[(2-amino-3-bromo-7-quinolyl)oxymethyl]-1-(4-aminopyrrolo[2,3-d]pyrimidin-7-yl)bicyclo[3.1.0]hexane-2,3-diol (17) (2.6 mg, 5.1 μmol, 43.4% yield, 98.08% purity) as a white solid. LCMS: (ESI): m/z calcd, for C₂₂H₂₂BrN₆O₃ 499.09 [M+H]⁺, found 499.1. ¹H NMR (400 MHz, CD₃OD) δ: ppm 8.21 (s, 1H), 8.03 (s, 1H), 7.56 (d, J=8.8 Hz, 1H), 7.15 (d, J=3.7 Hz, 1H), 7.11 (d, J=2.2 Hz, 1H), 6.98 (dd, J=8.8, 2.4 Hz, 1H), 6.51 (d, J=3.7 Hz, 1H), 4.78 (br d, J=6.4 Hz, 1H), 4.43-4.52 (m, 2H), 4.11 (d, J=6.4 Hz, 1H), 2.60 (t, J=6.1 Hz, 1H), 1.94-1.98 (m, 2H), 1.22-1.29 (m, 1H).

### Example 14

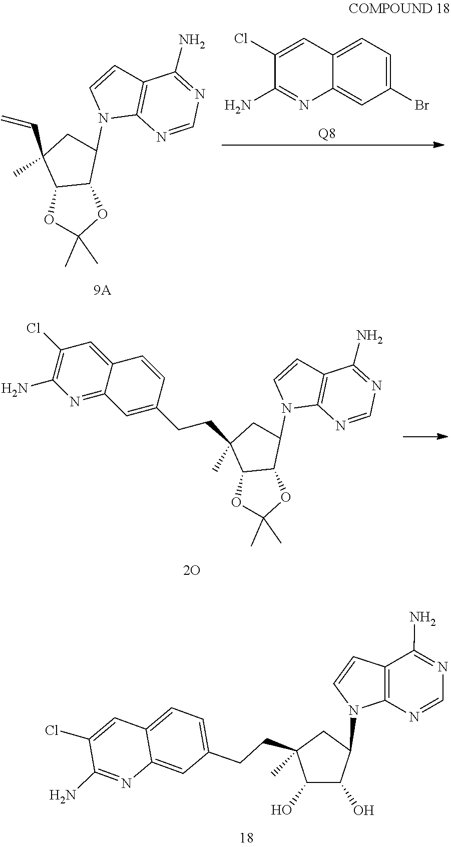

To a solution of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (200 mg, 636.17 μmol, 1 eq.) in THF (10 mL) was added 9-BBN dimer (338.7 mg, 1.40 mmol, 2.2 eq.) at 20° C. The reaction was stirred at 50° C. for 1 h. The mixture was cooled to 20° C., and a solution of K₃PO₄ (675.2 mg, 3.18 mmol, 5 eq.) in H₂O (2 mL) was added. The mixture stirred for 30 min, and then 7-bromo-3-chloroquinolin-2-amine (Q8) (213 mg, 827.0 μmol, 1.3 eq.) and Pd(dppf)Cl₂ (46.6 mg, 63.6 μmol, 0.1 eq.) were added at 20° C. The mixture was stirred at 70° C. for 15 h. The reaction progress was monitored by LCMS. Upon completion the mixture was filtered, diluted with brine (10 mL) and extracted with DCM (2×10 mL). The combined organic layers were dried over Na₂SO₄, filtered and concentrated under reduced pressure to give a residue. The residue was purified by column chromatography (SiO₂, DCM:MeOH=50:1 to 20:1) to give 20 (280 mg, 482.8 μmol, 75.9% yield, 85% purity) as a white solid. LCMS: (ESI): m/z calcd. for C₂₆H₃₀ClN₆O₂ 493.20 [M+H]⁺, found 493.3.

To a solution of 20 (280 mg, 567.95 μmol, 1 eq.) in THF (5 mL) was added HCl (4 M, 2.5 mL, 17.61 eq.). The mixture was stirred at 20° C. for 12 h. The mixture was concentrated under reduced pressure to give a residue. The crude product was triturated with CH₃CN:H₂O=10:1 (2×11 mL) at 25° C. for 30 min to give 18 (2 HCl, 218 mg, 406.3 μmol, 71.5% yield, 98% purity, 2 HCl) as a white solid. LCMS: (ESI): m/z calcd. for C₂₃H₂₆ClN₆O₂ 453.17 [M+H]⁺, found 453.3. ¹H NMR (400 MHz, CD₃OD) δ: 8.59 (s, 1H), 8.25 (s, 1H), 7.83 (d, J=8.3 Hz, 1H), 7.63-7.55 (m, 2H), 7.49 (d, J=8.3 Hz, 1H), 6.92 (d, J=3.7 Hz, 1H), 5.19-5.05 (m, 1H), 4.61-4.50 (m, 1H), 3.96 (d, J=6.1 Hz, 1H), 3.02-2.82 (m, 2H), 2.15-2.07 (m, 1H), 2.06-1.98 (m, 1H), 1.97-1.83 (m, 2H), 1.25 (s, 3H).

### Example 15

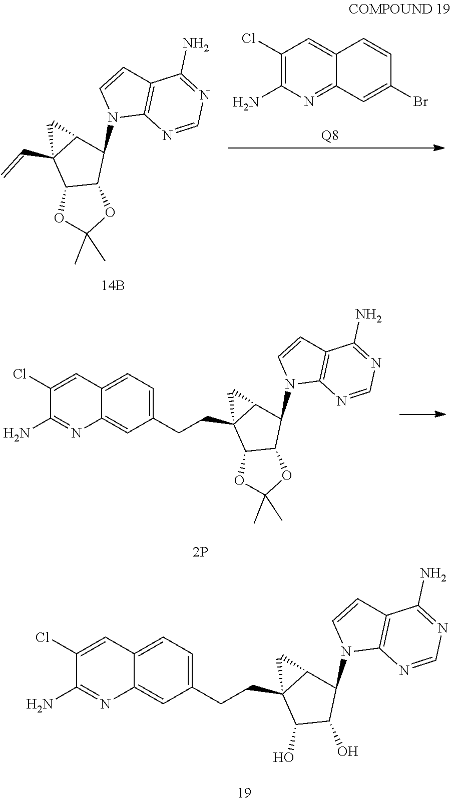

9-BBN dimer (193.7 mg, 800.3 μmol, 2.5 eq.) was added to a solution of 7-[(11S,12R,13S,14R,16S)-15,15-dimethyl-16-vinyl-21,22-dioxatricyclononan-12-yl]pyrrolo[2,3-d]pyrimidin-4-amine (14B) (100 mg, 320.1 μmol, 1 eq.) in THF (4 mL). The mixture was stirred at 50° C. for 2 h under N₂, and then cooled to 25° C. A solution of K₃PO₄ (339.8 mg, 1.60 mmol, 5 eq.) in H₂O (0.4 mL) were added. The mixture was stirred at rt for 0.5 h. 7-bromo-3-chloro-quinolin-2-amine (123.7 mg, 480.2 μmol, 1.5 eq.) and Pd(dppf)Cl₂ (23.4 mg, 32.0 μmol, 0.1 eq.) were added. The mixture was stirred at 70° C. for 12 h under N₂. The reaction progress was monitored by TLC (DCM:MeOH=10:1). Upon completion, the mixture was diluted with water (10 mL) and extracted with EA (2×10 mL). The combined organic layers were washed with brine (2×10 mL), dried over Na₂SO₄ and concentrated to give a crude product (600 mg). An additional reaction (100 mg of 14B) was performed using the above procedure, and 700 mg of crude product was obtained.

The crude products from two batches were combined and purified by column chromatography (SiO₂, DCM:MeOH=50:1 to 30:1) to afford 7-[2-[(20S,21R,22S,23R,25R)-21-(4-aminopyrrolo[2,3-d]pyrimidin-7-yl)-24,24-dimethyl-32,33-dioxatricyclononan-25-yl]ethyl]-3-chloro-quinolin-2-amine (2P) (240 mg, 87% purity, 427.2 μmol, 66% yield, average of 2 batches) as a yellow solid. LCMS: (ESI): m/z calcd. for C₂₆H₂₈ClN₆O₂ 491.19 [M+H]⁺, found 491.2.

To a solution of 2P (240 mg, 87% purity, 427.2 μmol, 1 eq.) in THF (5 mL) was added HCl (4 M, 2.5 mL). The mixture was stirred at 25° C. for 12 h. The reaction progress was monitored by TLC (DCM:MeOH=5:1). Upon completion, the mixture was filtered and concentrated under reduced pressure to give a residue. The residue was purified by prep-HPLC (column: Phenomenex Gemini-NX 150*30 mm*5 um; mobile phase: [water (0.04% NH₃H₂O+10 mM NH₄HCO₃)-ACN]; B %: 26%-56%, 8 min) to afford (1R,2R,3S,4R,5S)-1-[2-(2-amino-3-chloro-7-quinolyl)ethyl]-4-(4-aminopyrrolo[2,3-d]pyrimidin-7-yl)bicyclo[3.1.0]hexane-2,3-diol (19) (110 mg, 241.8 μmol, 57% yield) as a white solid. LCMS: (ESI): m/z calcd. for C₂₃H₂₄ClN₆O₂ 451.16 [M+H]⁺, found 451.2. ¹H NMR (400 MHz, CD₃OD) δ: 8.08 (d, J=11.5 Hz, 2H), 7.56 (d, J=8.3 Hz, 1H), 7.45 (s, 1H), 7.21 (dd, J=1.3, 8.3 Hz, 1H), 7.02 (d, J=3.5 Hz, 1H), 6.57 (d, J=3.8 Hz, 1H), 4.94 (s, 1H), 4.59 (s, 1H), 3.86 (d, J=6.5 Hz, 1H), 3.10-2.90 (m, 2H), 2.29-2.19 (m, 1H), 2.02-1.93 (m, 1H), 1.48-1.35 (m, 2H), 0.68 (br dd, J=5.4, 7.2 Hz, 1H).

### Example 16

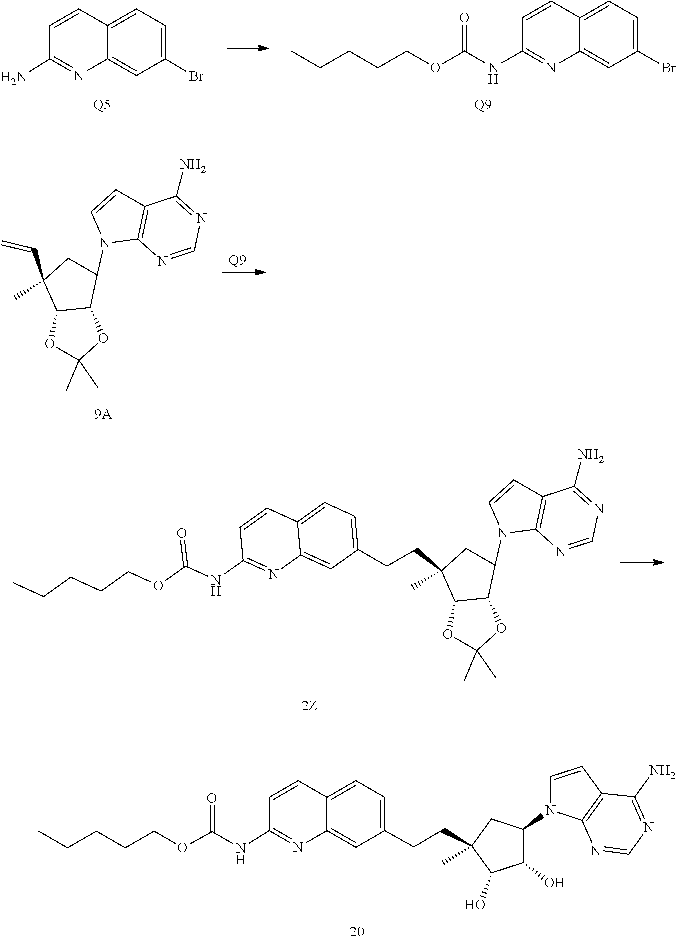

To a solution of 7-bromoquinolin-2-amine (Q5) (4.2 g, 16.03 mmol) in DCM (50 mL) were added A-methylimidazole (7.90 g, 96.20 mmol, 7.67 mL) and pentyl carbonochloridate (7.24 g, 48.10 mmol) at 0° C. The mixture was stirred at 20° C. for 12 h. The mixture was partitioned between DCM (30 mL) and brine (30 mL). The organic phase was separated, and the aqueous phase extracted with DCM (3×30 mL). The organic layers were combined and dried over Na₂SO₄. The solids were removed by filtration, and the filtrate was concentrated under reduced pressure. The crude was purified by column chromatography (SiO₂, PE:EA=0:1 to 20:1) to afford pentyl N-(7-bromo-2-quinolyl) carbamate (Q9) (3.2 g, 9.49 mmol, 59% yield, 100% purity) was obtained as a white solid. LCMS: (ESI): m/z calcd. for C₁₅H₁₈BrN₂O₂ 337.05 [M+H]⁺, found 337.1.

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (1 g, 3.18 mmol) and 9-BBN dimer (1.92 g, 7.95 mmol) in THF (30 mL) was stirred at 50° C. for 1.5 h under Ar and then cooled to 20° C. A solution of K₃PO₄ (3.38 g, 15.90 mmol) in H₂O (8 mL) was added. The mixture was stirred at 20° C. for 0.5 h. Pentyl N-(7-bromo-2-quinolyl)carbamate (1.29 g, 3.82 mmol) (Q9) and Pd(dppf)Cl₂ (232.75 mg, 318.09 μmol) were added. The mixture was stirred at 60° C. for 12 h under Ar. The mixture was partitioned between EA (30 mL) and water (30 mL). The organic phase was separated, and the aqueous phase washed with EA (3×30 mL). The organic layers were combined, washed with brine (30 mL) and dried over Na₂SO₄. The solids were removed by filtration, and the filtrate was concentrated under reduced pressure. The crude was purified by column chromatography (SiO₂, PE:EA=1:1 then DCM:MeOH=20:1) to afford pentyl (7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)quinolin-2-yl)carbamate (2Z) (2.01 g, 3.14 mmol, 98% yield, 89% purity) as a yellow solid. LCMS: (ESI): m/z calcd. for C₃₂H₄₁N₆O₄ 573.31 [M+H]⁺, found 573.5.

To a solution of pentyl N-[7-[2-[(3aR,4S,6R,6aS)-6-(4-aminopyrrolo[2,3-d]pyrimidin-7-yl)-2,2,4-trimethyl-3a,5,6,6a-tetrahydrocyclopenta[d][1,3]dioxol-4-yl]ethyl]-2-quinolyl]carbamate (2Z) (2.01 g, 3.14 mmol, 89% purity) in THF (10 mL) was added HCl (4 M, 8.96 mL). The mixture was stirred at 20° C. for 1 h. The mixture was concentrated under reduced pressure to afford a residue. The residue was purified by prep-HPLC (HCl condition, column: YMC-Triart Prep C18 150×40 mm×7 um; mobile phase: [water (0.225% HCl)-ACN]; B %: 22%-52%, 7.7 min) to afford pentyl A-[7-[2-[(1S,2R,3S,4R)-4-(4-aminopyrrolo[2,3-d]pyrimidin-7-yl)-2,3-dihydroxy-1-methyl-cyclopentyl]ethyl]-2-quinolyl]carbamate (20) (1.2 g, 1.98 mmol, 63% yield, 99% purity, 2 HCl) as an off-white solid. LCMS: (ESI): m/z calcd. for C₂₉H₃₇N₆O₄ 533.28 [M+H]⁺, found 533.5. ¹H NMR (400 MHz, CD₃OD) δ: 8.78 (d, J=9.0 Hz, 1H), 8.25 (s, 1H), 8.06 (d, J=8.4 Hz, 1H), 8.02 (s, 1H), 7.71 (d, J=8.2 Hz, 1H), 7.59 (d, J=3.5 Hz, 1H), 7.42 (d, J=9.3 Hz, 1H), 6.93 (d, J=3.5 Hz, 1H), 5.08-5.20 (m, 1H), 4.52-4.59 (m, 1H), 4.41 (t, J=6.6 Hz, 2H), 3.98 (d, J=6.2 Hz, 1H), 2.89-3.10 (m, 2H), 1.87-2.17 (m, 4H), 1.76-1.86 (m, 2H), 1.37-1.52 (m, 4H), 1.27 (s, 3H), 0.92-1.03 (m, 3H).

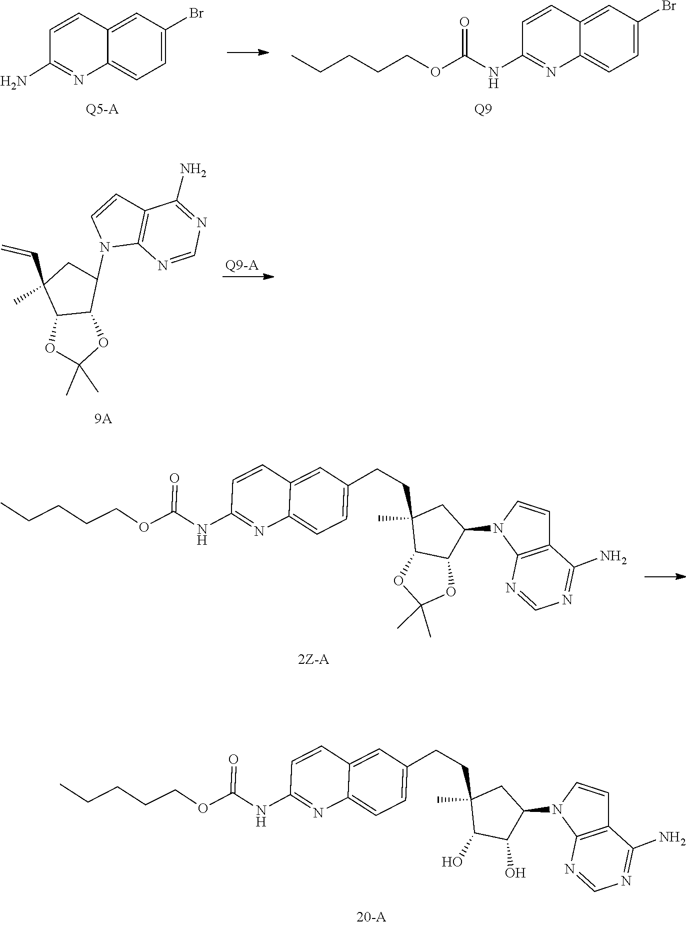

Pentyl carbonochloridate (202.54 mg, 1.34 mmol, 3 eq.) was added dropwise to a solution of 6-bromoquinolin-2-amine (Q5-A) (100 mg, 448.3 μmol, 1 eq.) and 1-methylimidazole (220.8 mg, 2.69 mmol, 214 μL) in DCM (2.5 mL) at 0° C. The mixture was stirred at 25° C. for 12 h. The reaction progress was monitored by TLC (PE:EA=1:1). Upon completion, the mixture was extracted with EA (2×5 mL). The combined organic layers were washed with brine (2×5 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜10% EA:PE gradient @ 18 mL/min) to afford pentyl N-(6-bromo-2-quinolyl) carbamate (Q9-A) (130 mg, 380.9 μmol, 84% yield) as a white solid. LCMS: (ESI): m/z calcd. for C₁₅H₁₈BrN₂O₂ 339.05 [M+H]⁺, found 338.8.

9-BBN dimer (192.45 mg, 795.21 μmol, 2.5 eq.) was added to a solution of 9A (100 mg, 318.09 μmol, 1 eq.) in THF (4 mL), and the mixture was stirred at 50° C. for 2 h under Ar. The mixture was cooled to rt, and then a solution of K₃PO₄ (337.59 mg, 1.59 mmol, 5 eq.) in H₂O (0.4 mL) were added. The mixture was stirred for 0.5 h, and then Q9-A (128.71 mg, 381.70 μmol, 1.2 eq.) and Pd(dppf)Cl₂ (23.27 mg, 31.81 μmol, 0.1 eq.) were added. The mixture was stirred at 70° C. for 12 h. The reaction progress was monitored by TLC (DCM:MeOH=10:1). Upon completion, the reaction mixture was extracted with EA (2×5 mL). The combined organic layers were washed with brine (2×5 mL), dried over Na₂SO₄, filtered and concentrated under reduced pressure to give a residue. The residue was purified by flash silica gel chromatography (ISCO®; 12 g SepaFlash® Silica Flash Column, Eluent of 0-5% DCM/MeOH @ 30 mL/min) to afford pentyl N-[7-[2-[(3aR,4S,6R,6aS)-6-(4-aminopyrrolo[2,3-d]pyrimidin-7-yl)-2,2,4-trimethyl-3a,5,6,6a-tetrahydrocyclopenta[d][1,3]dioxol-4-yl]ethyl]-2-quinolyl]carbamate (2Z-A) (110 mg, 79% purity, 151.7 μmol, 47% yield) as a yellow oil. LCMS: (ESI): m/z calcd. for C₃₂H₄₁N₆O₂ 573.31 [M+H]⁺, found 573.3.

To a solution of 2Z-A (110 mg, 79% purity, 151.7 μmol, 1 eq.) in THF (4 mL) was added HCl (4 M, 2 mL). The mixture was stirred at 25° C. for 12 h. The reaction progress was monitored by TLC (DCM:MeOH=5:1). Upon completion, the mixture was filtered and concentrated under reduced pressure to give a residue. The residue was purified by prep-HPLC (column: Phenomenex Gemini-NX 150*30 mm*5 um; mobile phase: [water (0.05% HCl)-ACN]; B %: 20%-42%, 5 min) to afford pentyl N-[7-[2-[(1S,2R,3S,4R)-4-(4-aminopyrrolo[2,3-d]pyrimidin-7-yl)-2,3-dihydroxy-1-methyl-cyclopentyl]ethyl]-2-quinolyl]carbamate (20-A) (68 mg, 111.6 μmol, 73% yield, 99.4% purity, 2 HCl salt) as a white solid. LCMS: (ESI): m/z calcd. for C₂₉H₃₇N₆O₄ 533.28 [M+H]⁺, found 533.3. ¹H NMR (400 MHz, CD₃OD) δ: 8.81 (d, J=9.0 Hz, 1H), 8.24 (s, 1H), 8.12 (d, J=8.6 Hz, 1H), 8.03-7.94 (m, 2H), 7.59 (d, J=3.7 Hz, 1H), 7.43 (d, J=9.3 Hz, 1H), 6.93 (d, J=3.7 Hz, 1H), 5.14 (td, J=8.3, 10.7 Hz, 1H), 4.58-4.52 (m, 1H), 4.42 (t, J=6.6 Hz, 2H), 3.98 (d, J=6.4 Hz, 1H), 3.05-2.87 (m, 2H), 2.15-1.98 (m, 2H), 1.92 (dt, J=5.4, 11.5 Hz, 2H), 1.86-1.77 (m, 2H), 1.52-1.38 (m, 4H), 1.26 (s, 3H), 1.00-0.94 (m, 3H).

### Example 17

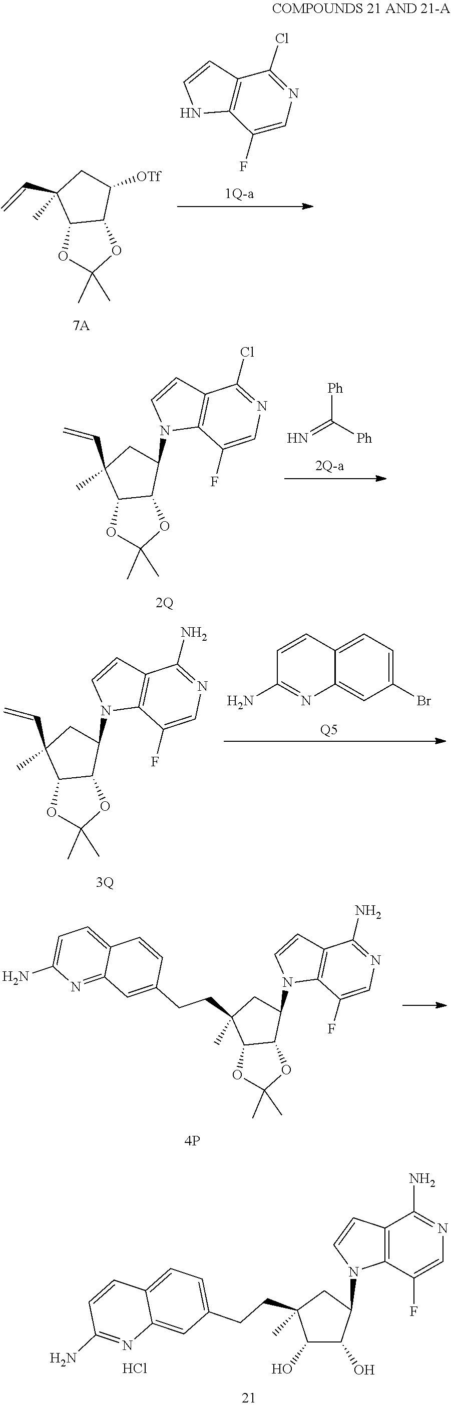

t-BuOK (951 mg, 8.48 mmol) was added to a mixture of 4-chloro-7-fluoro-1H-pyrrolo[3,2-c]pyridine (1Q-a) (1.55 g, 9.08 mmol) and DMF (15 mL). The mixture was stirred at rt for 30 min. Then [(3aR,4R,6S,6aR)-2,2,4-trimethyl-4-vinyl-3a,5,6,6a-tetrahydrocyclopenta[d][1,3]dioxol-6-yl] trifluoromethanesulfonate (7A) (2.0 g, 6.05 mmol) was added. The mixture was stirred at rt for 19 h. The mixture was diluted with water (20 mL) and then extracted with EA (2×20 mL). The organic layers were combined, washed with brine (10 mL) and dried over anhydrous Na₂SO₄. The solids were removed by filtration, and the filtrate was concentrated to afford a residue. The residue was purified by reversed-phase HPLC (A: 0.04% NH₃.H₂O, B: CH₃OH) to afford 4-chloro-7-fluoro-1-((3aS,4R,6R,6aR)-2,2,6-trimethyl-6-vinyltetrahydro-4H-cyclopenta[d][1,3]dioxol-4-yl)-1H-pyrrolo[3,2-c]pyridine (2Q) (710 mg, 33% yield) as a yellow gum. LCMS: (ESI): m/z calcd. for C₁₈H₂₁ClFN₂O₂ 351.13 [M+H]⁺, found 351.1.

A mixture of 2Q (70.2 mg, 0.2 mmol), diphenylmethanimine (54.4 mg, 0.300 mmol, 0.050 mL), BINAP (24.9 mg, 40.0 μmol), Pd₂(dba)₃ (18.3 mg, 0.020 mmol) and t-BuONa (38.4 mg, 0.400 mmol) in toluene (2 mL) was degassed under vacuum and purged with Ar. The mixture was stirred at 110° C. for 18 h. The reaction was quenched with NH₄Cl (sat., aq., 1 mL). The mixture was diluted with EA (20 mL) and washed with brine (10 mL). The organic layer was dried over anhydrous Na₂SO₄ and concentrated to afford a crude imine intermediate. The crude intermediate was dissolved in CH₃OH (3 mL). Hydroxylamine (64 mg, 50% solution in water) was added at rt. The mixture was stirred at rt for 1 h. The mixture was concentrated under reduced pressure. The residue was purified by silica gel chromatography (PA:EA=2:1, 200 mL; then DCM:MeOH=20:1) to afford 1-[(3aR,4R,6R,6aS)-2,2,4-trimethyl-4-vinyl-3a,5,6,6a-tetrahydrocyclopenta[d][1,3]dioxol-6-yl]-7-fluoro-pyrrolo[3,2-c]pyridin-4-amine (3Q) (40 mg, 60% yield over 2 steps) as a yellow gum. LCMS: (ESI): m/z calcd. for C₁₈H₂₃FN₃O₂ 332.18 [M+H]⁺, found 332.1.

9-BBN dimer (255.61 mg, 1.06 mmol) was added to a solution of 3Q (140 mg, 422.47 μmol) in THF (4 mL). The mixture was stirred at 50° C. for 2 h under Ar. The mixture was cooled to 25° C., and then a solution of K₃PO₄ (448.38 mg, 2.11 mmol) in H₂O (0.4 mL) were added. The mixture was stirred for 0.5 h. 7-bromoquinolin-2-amine (Q5) (122.51 mg, 549.21 μmol) and Pd (dppf)Cl₂ (30.91 mg, 42.25 μmol) were added. The mixture was stirred at 70° C. for 12 h under Ar. The mixture was filtered and concentrated under reduced pressure. The residue was purified by reverse-phase column (C18: 0%˜70% water (0.5 mL NH₃.H₂O in 1 L H₂O)/CH₃CN at 40 mL/min) to afford 7-[2-[(3aR,4S,6R,6aS)-6-(4-amino-7-fluoro-pyrrolo[3,2-c]pyridin-1-yl)-2,2,4-trimethyl-3a,5,6,6a-tetrahydrocyclopenta[d][1,3]dioxol-4-yl]ethyl]quinolin-2-amine (4P) (110 mg, 0.212 mmol, 50% yield, 92% purity) as a yellow solid. LCMS: (ESI): m/z calcd. for C₂₇H₃₁FN₅O₂ 476.24 [M+H]⁺, found 476.2.

HCl (4 M, 2 mL) was added to a solution of 4P (110 mg, 212.80 μmol) in THF (4 mL). The mixture was stirred at 25° C. for 12 h. The mixture was filtered and then concentrated under reduced pressure. The residue was purified by prep-HPLC (column: Phenomenex Gemini-NX 150×30 mm×5 um; mobile phase: [water (0.05% HCl)-ACN]; B %: 5%-35%, 7 min) to afford (1S,2R,3S,5R)-5-(4-amino-7-fluoro-pyrrolo[3,2-c]pyridin-1-yl)-3-[2-(2-amino-7-quinolyl)ethyl]-3-methyl-cyclopentane-1,2-diol (21) as a hydrochloride salt (white solid, 62 mg, 120.46 μmol, 56% yield, 98.78% purity). LCMS: (ESI): m/z calcd. for C₂₄H₂₇FN₅O₂. 436.21 [M+H]⁺, found 436.2. ¹H NMR (400 MHz, CDCl₃) δ: 8.31 (d, J=9.3 Hz, 1H), 7.82 (d, J=8.2 Hz, 1H), 7.73 (d, J=3.5 Hz, 1H), 7.60 (d, J=6.4 Hz, 1H), 7.53 (s, 1H), 7.45 (dd, J=1.3, 8.1 Hz, 1H), 7.13 (dd, J=2.0, 3.2 Hz, 1H), 7.01 (d, J=9.3 Hz, 1H), 5.19-5.11 (m, 1H), 4.45-4.40 (m, 1H), 3.93 (d, J=6.4 Hz, 1H), 2.98-2.82 (m, 2H), 2.17 (dd, J=8.2, 12.9 Hz, 1H), 1.95-1.80 (m, 3H), 1.24 (s, 3H).

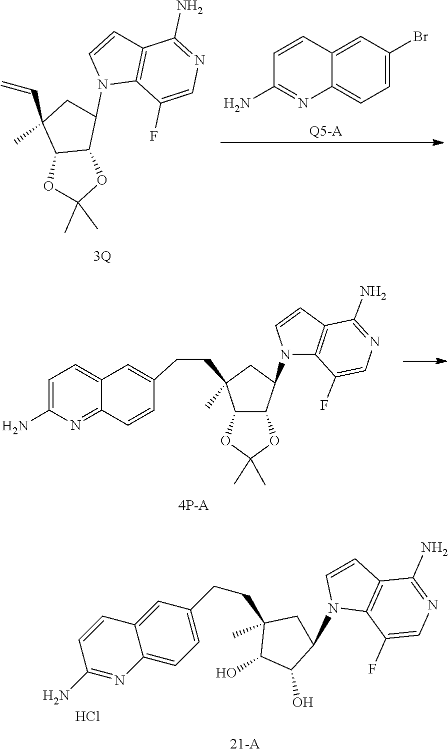

9-BBN dimer (182.6 mg, 754.4 μmol, 2.5 eq.) was added to a solution of 3Q (100 mg, 301.8 μmol, 1 eq.) in THF (4 mL), and the mixture was stirred at 50° C. for 2 h under Ar. The mixture was cooled to 25° C., and then a solution of K₃PO₄ (320.3 mg, 1.51 mmol, 5 eq.) in H₂O (0.4 mL) were added. The mixture was stirred for 0.5 h. 7-bromoquinolin-2-amine (Q5-A) (87.5 mg, 392.3 μmol, 1.3 eq.) and Pd (dppf)Cl₂ (22.1 mg, 30.2 μmol, 0.1 eq.) were added. The mixture was stirred at 70° C. for 12 h under Ar. The reaction progress was monitored by TLC (DCM:MeOH=10:1). Upon completion, the mixture was filtered and concentrated under reduced pressure to give a residue. The residue was purified by reversed-phase HPLC (40 g C-18 column: chromatography (10%˜70% water (0.5 mL NH₃.H₂O in 1 L H₂O)/CH₃CN @ 40 mL/min) to afford 7-[2-[(3aR,4S,6R,6aS)-6-(4-amino-7-fluoro-pyrrolo[3,2-c]pyridin-1-yl)-2,2,4-trimethyl-3a,5,6,6a-tetrahydrocyclopenta[d][1,3]dioxol-4-yl]ethyl]quinolin-2-amine (4P-A) (100 mg, 92.8% purity, 193.5 μmol, 64% yield) as a yellow solid. LCMS: (ESI): m/z calcd. for C₂₇H₃₁FN₅O₂ 476.24 [M+H]⁺, found 476.3.

HCl (4 M, 2 mL) was added to a solution of 4P-A (100 mg, 92.8% purity, 193.5 μmol, 1 eq.) in THF (4 mL). The mixture was stirred at 25° C. for 12 h. The reaction progress was monitored by TLC (DCM:MeOH=10:1). Upon completion, the mixture was filtered and concentrated under reduced pressure to give a residue. The residue was purified by prep-HPLC (column: Phenomenex Gemini-NX 150*30 mm*5 um; mobile phase: [water (0.05% HCl)-ACN]; B %: 0%-30%, 7 min) to afford (1S,2R,3S,5R)-5-(4-amino-7-fluoro-pyrrolo[3,2-c]pyridin-1-yl)-3-[2-(2-amino-7-quinolyl)ethyl]-3-methyl-cyclopentane-1,2-diol (21-A) as a hydrochloride salt (white solid, 62 mg, 0.122 mmol, 63% yield, 100% purity). LCMS: (ESI): m/z calcd. for C₂₄H₂₇FN₅O₂ 436.21 [M+H]⁺, found 436.3. ¹H NMR (400 MHz, CDCl₃) δ: 8.31 (d, J=9.3 Hz, 1H), 7.76 (s, 1H), 7.75-7.69 (m, 2H), 7.62-7.58 (m, 2H), 7.14-7.12 (m, 1H), 7.06 (d, J=9.3 Hz, 1H), 5.19-5.11 (m, 1H), 4.45-4.40 (m, 1H), 3.93 (d, J=6.3 Hz, 1H), 2.94-2.78 (m, 2H), 2.16 (dd, J=8.3, 13.1 Hz, 1H), 1.93-1.78 (m, 3H), 1.23 (s, 3H).

### Example 18

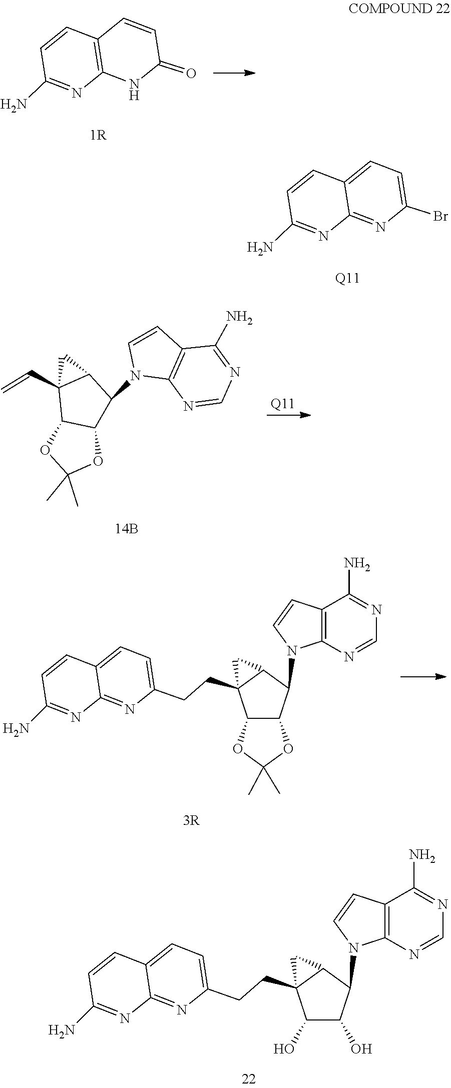

A mixture of 7-amino-1H-1,8-naphthyridin-2-one (1R) (1 g, 6.20 mmol, 1 eq.) and POBr₃ (5.34 g, 18.61 mmol, 1.89 mL, 3 eq.) in MeCN (10 mL) was refluxed for 3 h under Ar atmosphere. The reaction progress was monitored by LCMS. Upon completion, the mixture was cooled to rt, and the reaction quenched by ice-water (20 mL). The mixture was neutralized by NH₄OH to pH>8. The precipitated solid was filtered and washed with water. The filter cake was triturated with MeOH (50 mL) at rt for 30 min. The insoluble solid was filtered off. The filtrate was concentrated under reduced pressure to give a residue. The residue was purified by column chromatography (SiO₂, DCM:MeOH=50:1 to 20:1). 7-bromo-1,8-naphthyridin-2-amine (Q11) (90 mg, 5.6% yield, 89% purity) was obtained as a yellow solid. LCMS: (ESI): m/z calcd. for C₈H₇BrN₃ 225.97 [M+H]⁺, found 226.1.

7-[(12R,13S,14R,16S)-15,15-dimethyl-16-vinyl-21,22-dioxatricyclononan-12-yl]pyrrolo[2,3-d]pyrimidin-4-amine (14B) (100 mg, 320.1 μmol, 1 eq.) in THF (5 mL) was added 9-BBN dimer (170.5 mg, 704.3 μmol, 2.2 eq.) at 20° C. The mixture was stirred at 50° C. for 60 min, and then cooled to 20° C. K₃PO₄ (339.8 mg, 1.60 mmol, 5 eq.) in H₂O (1 mL) was added, and the mixture and stirred for 30 min. Q11 (86.1 mg, 384.17 μmol, 1.2 eq.) and Pd(dppf)Cl₂ (23.4 mg, 32.01 μmol, 0.1 eq.) were added. The reaction was degassed for 3 times and then stirred at 70° C. for 15 h. The reaction progress was monitored by LCMS. Upon completion, the mixture was diluted with brine (10 mL) and extracted with EA (3×20 mL). The combined organic layers were dried over Na₂SO₄, filtered and concentrated under reduced pressure to give a residue. The residue was purified by reversed-phase HPLC (0.05% NH₃.H₂O condition) to give 7-[2-[(20R,21S,22R,24R)-20-(4-aminopyrrolo[2,3-d]pyrimidin-7-yl)-23,23-dimethyl-32,33-dioxatricyclononan-24-yl]ethyl]-1,8-naphthyridin-2-amine (3R) (81 mg, 173.4 μmol, 54.2% yield, 97.9% purity) as a yellow solid. LCMS: (ESI): m/z calcd. for C₂₅H₂₈N₇O₂ 458.22 [M+H]⁺, found 458.2.

To a solution of 3R (81 mg, 173.4 μmol, 97.9% purity, 1 eq.) in THF (6 mL) was added HCl (4 M, 3 mL). The mixture was stirred at 20° C. for 4 h. The reaction progress was monitored by LCMS. Upon completion, the mixture was concentrated under reduced pressure to afford a residue. The residue was neutralized by NH₄OH to pH>8 and then purified by prep-HPLC (column: YMC Triart C18 150*25 mm*5 um; mobile phase: [water (10 mM NH₄HCO₃)-ACN]; B %: 21%-51%) to afford (1R,2R,3S,4R)-1-[2-(7-amino-1,8-naphthyridin-2-yl)ethyl]-4-(4-aminopyrrolo[2,3-d]pyrimidin-7-yl)bicyclo[3.1.0]hexane-2,3-diol (22) (37 mg, 88.5 μmol, 51% yield, 99.87% purity) as a white solid. LCMS: (ESI): m/z calcd. for C₂₂H₂₄N₇O₂ 418.19 [M+H]⁺, found 418.2. ¹H NMR (400 MHz, CD₃OD) δ: 8.08 (s, 1H), 8.00 (d, J=7.8 Hz, 1H), 7.92 (d, J=8.8 Hz, 1H), 7.22 (d, J=7.8 Hz, 1H), 7.06 (d, J=3.4 Hz, 1H), 6.85 (d, J=8.8 Hz, 1H), 6.58 (d, J=3.7 Hz, 1H), 4.94 (s, 1H), 4.63 (br d, J=6.8 Hz, 1H), 3.87 (d, J=6.6 Hz, 1H), 3.06-3.23 (m, 2H), 2.40 (ddd, J=14.2, 9.2, 5.4 Hz, 1H), 2.04 (ddd, J=13.9, 9.7, 7.0 Hz, 1H), 1.34-1.45 (m, 2H), 0.58 (br dd, J=7.0, 5.3 Hz, 1H).

### Example 19

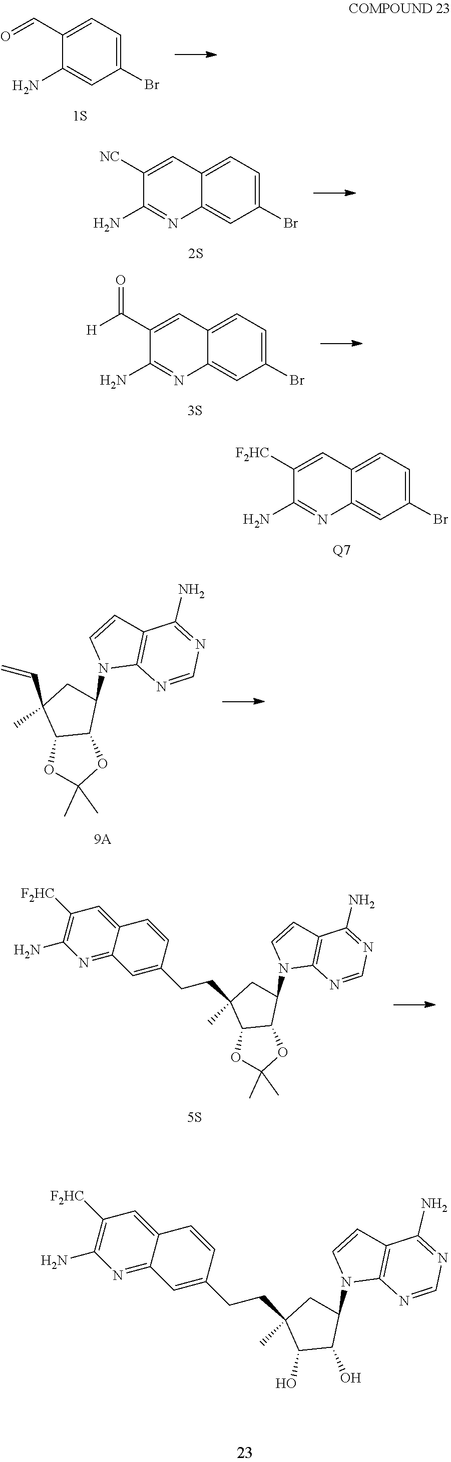

Piperidine (42.6 mg, 0.5 mmol, 50 μL, 0.1 eq.) was added to a mixture of 2-amino-4-bromo-benzaldehyde (1.0 g, 5.00 mmol, 1 eq.) and malononitrile (396.3 mg, 6.0 mmol, 1.2 eq.) in EtOH (20 mL). The mixture was refluxed for 4 h, and the product precipitated as a yellow solid. The reaction progress was monitored by LCMS. Upon completion, the solid was filtered. The collected solid was washed successively with EtOH (5 mL), MeOH (10 mL) and MTBE (10 mL). The mixture was dried under high vacuum to afford 2-amino-7-bromo-quinoline-3-carbonitrile (2S) (1.2 g, 4.84 mmol, 96.8% yield) as a yellow solid, which was used in the next step without further purification. LCMS: (ESI): m/z calcd. for C₁₀H₇BrN₃ 249.98 [M+H]⁺, found 250.1.

A solution of DIBAL-H (1 M in toluene, 3.30 mL, 3.3 eq.) was added dropwise to a mixture of 2S (248.1 mg, 1.0 mmol, 1 eq.) in DCM (10 mL) at −78° C. under N₂. After completion of the addition, the mixture was stirred at −78° C. for 4 h. The flask was then transformed to an ice-water bath, and the mixture was stirred at 0° C. for 30 min. The reaction was quenched by 2 M HCl to pH=3. The mixture was extracted with DCM:MeOH (10:1, 6×30 mL). The separated organic layers were combined, washed with brine (10 mL), dried over anhydrous Na₂SO₄, and concentrated to afford crude 2-amino-7-bromo-quinoline-3-carbaldehyde (3S) (146 mg, 44% purity) as a yellow solid, which was used in the next step without further purification.

To a solution of 3S (146 mg) in DCM (6 mL) was added DAST (562.4 mg, 3.49 mmol, 461 μL, 6 eq.) at 0° C. The mixture was stirred at rt for 16 h. The reaction progress was monitored by LCMS. Upon completion, the reaction was quenched by sat. aq. solution of NaHCO₃ (10 mL). The mixture was diluted with water (10 mL) and then extracted with EA (2×20 mL). The separated organic layers were combined, washed with brine (10 mL), dried over anhydrous Na₂SO₄, and concentrated to afford a brown residue. The residue was purified by reversed pre-HPLC (0.05% NH₃.H₂O condition) to afford 7-bromo-3-(difluoromethyl)quinolin-2-amine (Q7) (34 mg, 12% yield over two steps) as a yellow solid. LCMS: (ESI): m/z calcd. for C₁₀H₈BrF₂N₂ 274.98 [M+H]⁺, found 274.9.

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (150 mg, 477.1 μmol, 1 eq.) and 9-BBN dimer (254.0 mg, 1.05 mmol, 2.2 eq.) in dry THF (5 mL) was stirred at 50° C. for 2 h under Ar. The mixture was cooled to rt. A solution of K₃PO₄ (506.40 mg, 2.39 mmol, 5 eq.) in H₂O (0.5 mL) was added. The mixture was stirred at rt for 0.5 h. 7-bromo-3-(difluoromethyl)quinolin-2-amine (Q7) (169.4 mg, 620 μmol, 1.3 eq.) and Pd(dppf)Cl₂ (34.9 mg, 47.7 μmol, 0.1 eq.) were added. The mixture was stirred at 70° C. for 12 h under Ar. The reaction progress was monitored by LCMS. Upon completion, the mixture was diluted with brine (15 mL) and extracted with EA (4×20 mL). The separated organic layers were combined, dried over anhydrous Na₂SO₄, and concentrated to afford a residue. The residue was purified by silica gel chromatography (PE:EA=1:0 to 1:1, and then DCM:MeOH=10:0 to 10:1) to afford 5S (190 mg, 367.6 μmol, 77% yield, 98.4% purity) as light yellow solid. LCMS: (ESI): m/z calcd. for C₂₇H₃₁F₂N₆O₂ 509.25 [M+H]⁺, found 509.4.

A mixture of 5S (185 mg, 363.8 μmol, 1 eq.), HCl (4 M, 2 mL), and THF (4 mL) was stirred at 20° C. for 16 h. The mixture was concentrated under high vacuum to afford a residue. The residue was dissolved in MeCN:H₂O (1:1, 1 mL). MeCN (4 mL) was added dropwise to the mixture. The precipitated light yellow solid was collected by filtration and washed with MeCN:H₂O (10:1.3 mL) to afford Compound 23 as a hydrochloride salt (light yellow solid, 126 mg, 63.6% yield, 99.3% purity). LCMS: (ESI): m/z calcd. for C₂₄H₂₇F₂N₆O₂ 469.22 [M+H]⁺, found 469.2. ¹H NMR (400 MHz, CD₃OD) δ: 8.66 (s, 1H), 8.25 (s, 1H), 7.94 (d, J=8.3 Hz, 1H), 7.61 (s, 1H), 7.57 (d, J=3.5 Hz, 1H), 7.51 (d, J=8.3 Hz, 1H), 7.21-6.89 (m, 2H), 5.17-5.06 (m, 1H), 4.59-4.52 (m, 1H), 3.96 (d, J=6.3 Hz, 1H), 3.05-2.82 (m, 2H), 2.14-1.98 (m, 2H), 1.98-1.83 (m, 2H), 1.25 (s, 3H). ¹⁹F NMR (376 MHz, CD₃OD) δ: 120.5.

### Example 20

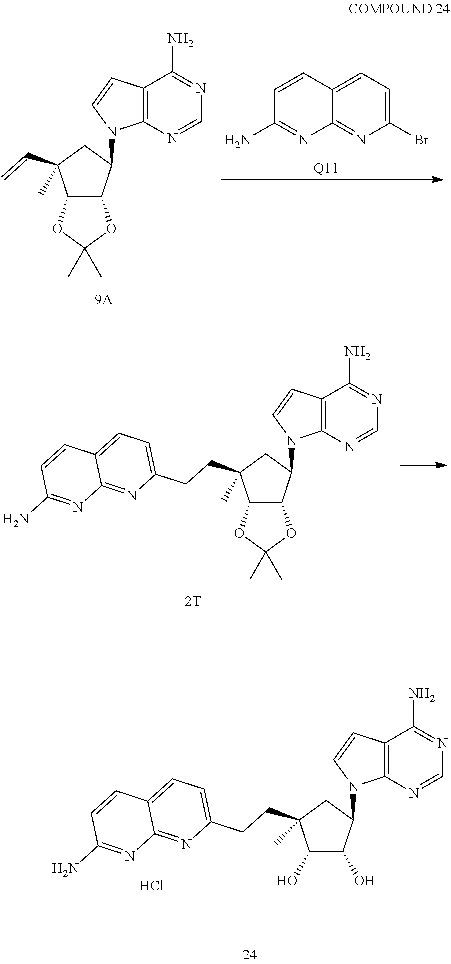

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (150 mg, 477.1 μmol, 1 eq.) and 9-BBN dimer (288.7 mg, 1.19 mmol, 2.5 eq.) in THF (5 mL) was stirred at 50° C. for 2 h under Ar. The mixture was then cooled to rt. A solution of K₃PO₄ (506.4 mg, 2.39 mmol, 5 eq.) in H₂O (1 mL) was added. The mixture was stirred at rt for 30 min. 7-bromo-1, 8-naphthyridin-2-amine (Q11) (139.0 mg, 620.3 μmol, 1.3 eq.) and Pd(dppf)Cl₂ (34.9 mg, 47.7 μmol, 0.1 eq.) were added. The mixture was stirred at 70° C. for 16 h under Ar. The reaction progress was monitored by LCMS. Upon completion, the mixture was diluted with H₂O (10 mL) and extracted with EA (3×20 mL). The combined organic layers were washed with brine (10 mL), dried over anhydrous Na₂SO₄, filtered and concentrated under reduced pressure to give a residue. The residue was purified by prep-HPLC (0.05% NH₃.H₂O condition) to afford 2T (122 mg, 259.3 μmol, 54.4% yield, 97.7% purity) as a yellow gum. LCMS: (ESI): m/z calcd. for C₂₅H₃₀N₇O₂ 460.24 [M+H]⁺, found 460.3.

To a mixture of 2T (122 mg, 259.3 μmol, 54.4% yield, 97.7% purity) in THF (6 mL) was added HCl (4 M, 3 mL). The mixture was stirred at rt for 12 h. The reaction progress was monitored by LCMS. Upon completion, the mixture was concentrated under reduced pressure to afford a residue. The residue was triturated with MeCN:water (10:1, 10 mL) at 20° C. for 30 min. The solid was collected by filtration and then suspended in MeCN:water (1:1, 1 mL). MeCN:water (10:1, 5 mL) was added dropwise. The solid was filtered and washed with MeCN:water (10:1, 5 mL) to afford Compound 24 as a hydrochloride salt (white solid, 83 mg, 167 μmol, 64.4% yield, and 99.1% purity). LCMS: (ESI): m/z calcd. for C₂₂H₂₆N₇O₂ 420.21 [M+H]⁺, found 420.3. ¹H NMR (400 MHz, CD₃OD) δ: 8.37 (d, J=8.1 Hz, 1H), 8.21-8.27 (m, 2H), 7.56 (d, J=3.7 Hz, 1H), 7.50 (d, J=8.1 Hz, 1H), 7.07 (d, J=9.0 Hz, 1H), 6.91 (d, J=3.7 Hz, 1H), 5.06-5.19 (m, 1H), 4.54 (t, J=7.0 Hz, 1H), 3.97 (d, J=6.4 Hz, 1H), 2.97-3.20 (m, 2H), 1.93-2.15 (m, 4H), 1.24 (s, 3H).

### Example 21

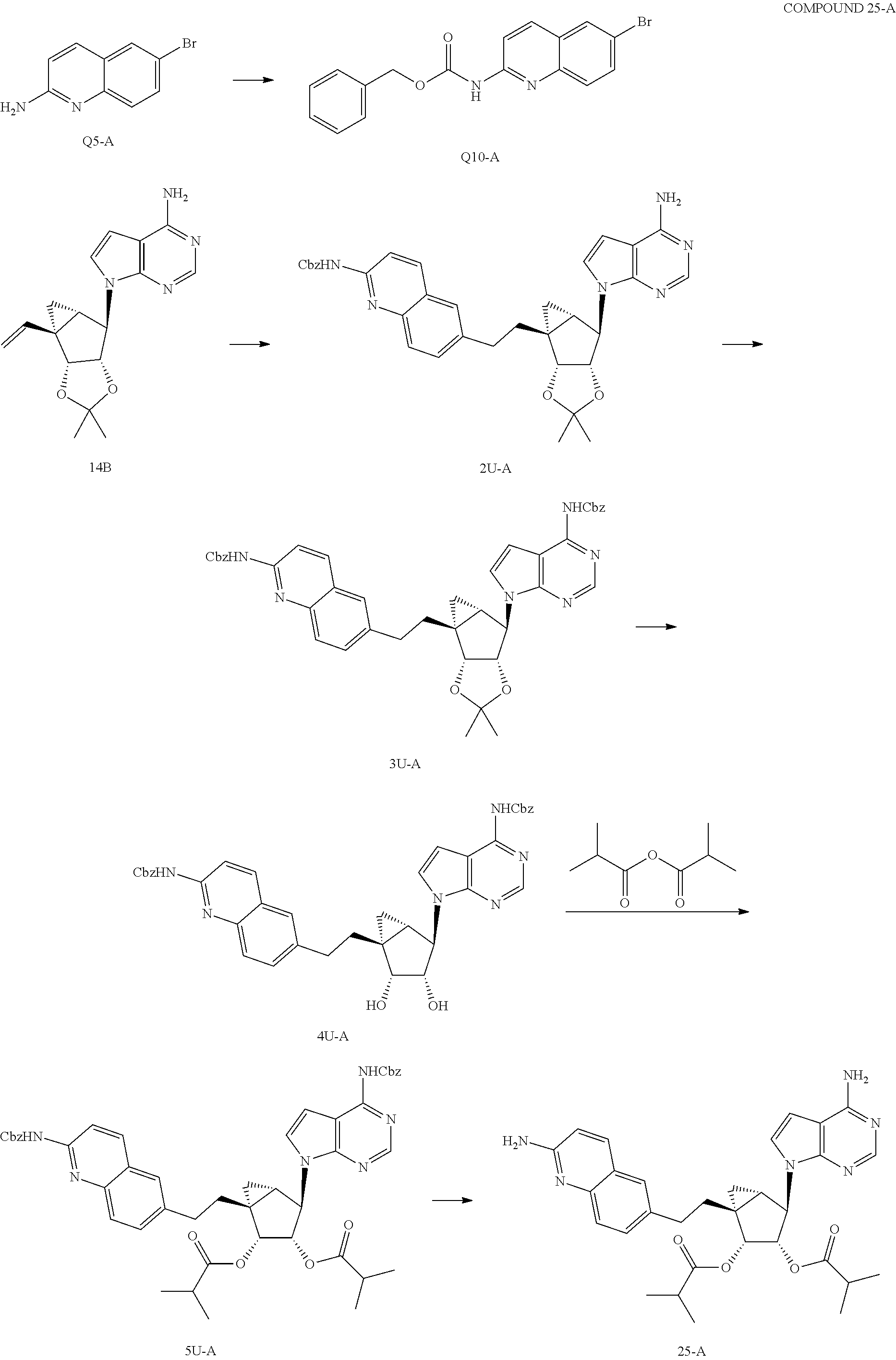

CbzCl (1.84 g, 10.76 mmol, 1.53 mL) was added dropwise to a solution of 1 (600 mg, 2.69 mmol) and 1-methylimidazole (1.77 g, 21.52 mmol, 1.72 mL) in DCM (15 mL) at 0° C. The mixture was stirred at 25° C. for 12 h. The reaction was quenched with NaHCO₃ (sat., aq., 10 mL) and extracted with EA (2×10 mL). The combined organic layers were washed with brine (10 mL×2), dried over Na₂SO₄. The solids were removed by filtration, and the filtrate was concentrated under reduced pressure. The residue was purified by silica column chromatography (PE:EA=20:1 to 10:1) to afford benzyl N-(6-bromo-2-quinolyl)carbamate (Q10-A) (780 mg, 2.14 mmol, 76% yield) as a white solid. LCMS: (ESI): m/z calcd. for C₁₇H₁₄BrN₂O₂ 357.02 [M+H]⁺, found 357.0.

A mixture of 7-[(11S,12R,13S,14R,16S)-15,15-dimethyl-16-vinyl-21,22-dioxatricyclononan-12-yl]pyrrolo[2,3-d]pyrimidin-4-amine (14B) (200 mg, 0.640 mmol) and 9-BBN dimer (387.4 mg, 1.60 mmol) in THF (8 mL) was stirred at 50° C. for 2 h under Ar and then cooled to rt. A solution of K₃PO₄ (679.54 mg, 3.20 mmol) in H₂O (0.8 mL) was added. The mixture was stirred at rt for 0.5 h. Compound Q10-A (297.3 mg, 0.832 mol) and Pd(dppf)Cl₂ (46.9 mg, 0.064 mmol) were added. The mixture was stirred at 70° C. for 12 h under Ar. The mixture was filtered and concentrated under reduced pressure. The residue was purified by silica column chromatography (PE:EA=3:1 to 1:1 and DCM:MeOH=100:1 to 20:1) to afford benzyl (6-(2-((3aR,3bR,4aS,5R,5aS)-5-(4-amino-7H-pyrrolo[2,3-d]pyrimidin-7-yl)-2,2-dimethyltetrahydrocyclopropa[3,4]cyclopenta[1,2-d][1,3]dioxol-3b(3a/7)-yl)ethyl)quinolin-2-yl)carbamate (2U-A) (273 mg, 0.374 mmol, 58%) as a yellow solid. LCMS: (ESI): m/z calcd. for C₃₄H₃₅N₆O₄ 591.26 [M+H]⁺, found 591.2.

CbzCl (369.68 mg, 2.17 mmol, 0.308 mL) was added dropwise to a solution of 2U-A (320 mg, 0.542 mmol) and 1-methylimidazole (355.84 mg, 4.33 mmol, 0.345 mL) in DCM (8 mL) at 0° C. The mixture was stirred at rt for 12 h, and then quenched by addition of NaHCO₃ (sat., aq., 5 mL). The mixture was extracted with DCM (3×5 mL). The combined organic layers were washed with brine, dried over Na₂SO₄. The solids were removed by filtration, and the filtrate was concentrated under reduced pressure. The residue was purified by silica column chromatography (PE:EA=1:1 to DCM:MeOH=50:1 to 20:1) to afford benzyl (7-((3aR,3bR,4aS,5R,5aS)-3b-(2-(2-(((benzyloxy)carbonyl)amino)quinolin-6-yl)ethyl)-2,2-dimethylhexahydrocyclopropa[3,4]cyclopenta[1,2-d][1,3]dioxol-5-yl)-7H-pyrrolo[2,3-d]pyrimidin-4-yl)carbamate (3U-A) (158 mg, 0.202 mmol, 37% yield) as a white oil. LCMS: (ESI): m/z calcd. for C₄₂H₄₁N₆O₆ 725.3 [M+H]⁺, found 725.4.

To a solution of 3U-A (158 mg, 0.202 mmol) in THF (2 mL) was added HCl (4 M, 0.929 mL). The mixture was stirred at 25° C. for 3 h. The reaction was quenched by NH₃.H₂O (1 mL) and extracted with EA (2×5 mL). The combined organic layers were washed with brine (2×5 mL), dried over Na₂SO₄, filtered and concentrated under reduced pressure to give benzyl (7-((1S,2R,3S,4R,5R)-5-(2-(2-(((benzyloxy)carbonyl)amino)quinolin-6-yl)ethyl)-3,4-dihydroxybicyclo[3.1.0]hexan-2-yl)-7H-pyrrolo[2,3-d]pyrimidin-4-yl)carbamate (4U-A) (143 mg, crude) as a colorless solid. LCMS: (ESI): m/z calcd. for C₃₉H₃₇N₆O₆ 685.27 [M+H]⁺, found 685.3.

Isobutyric anhydride (160.8 mg, 1.02 mmol, 169 μL) was added to a solution of 4U-A (232 mg, 0.339 mmol), TEA (205.7 mg, 2.03 mmol, 0.283 mL) and DMAP (4.1 mg, 0.034 mmol) in DMF (5 mL). The mixture was stirred at 60° C. for 3 h, and then quenched with NaHCO₃ (sat., aq., 5 mL) and extracted with EA (3×5 mL). The combined organic layers were washed with brine (2×5 mL), dried over Na₂SO₄. The solids were removed by filtration, and the filtrate was concentrated under reduced pressure. The residue was purified by silica gel chromatography (0˜50% EA:PE gradient) to afford (1R,2R,3S,4R,5S)-4-(4-(((benzyloxy)carbonyl)amino)-7H-pyrrolo[2,3-d]pyrimidin-7-yl)-1-(2-(2-(((benzyloxy)carbonyl)amino)quinolin-6-yl)ethyl)bicyclo[3.1.0]hexane-2,3-diyl bis(2-methylpropanoate) (5U-A) (157 mg, 0.169 mmol, 49%) as a white solid. LCMS: (ESI): m/z calcd. for C₄₇H₄₉N₆O₈ 825.35 [M+H]⁺, found 825.4.

To a solution of 5U-A (187 mg, 0.226 mmol) in EtOH (5 mL) and THF (2 mL) was added Pd/C (100 mg, 10% wt). The mixture was degassed under reduced pressure, purged with H₂ and then stirred under H₂ atmosphere (15 psi) at 25° C. for 20 h. The mixture was filtered through a Celite pad to remove the Pd/C. The filtrate was concentrated under reduced pressure to afford the residue. The residue was purified by prep-HPLC (column: YMC Triart C18 150×25 mm×5 μm; mobile phase: [water (10 mM NH₄HCO₃)-ACN]; B %: 42%-72%, 9.5 min) to afford (1R,2R,3S,4R,5S)-4-(4-amino-7H-pyrrolo[2,3-d]pyrimidin-7-yl)-1-(2-(2-aminoquinolin-6-yl)ethyl)bicyclo[3.1.0]hexane-2,3-diyl bis(2-methylpropanoate) (25-A) (82 mg, 0.147 mmol, 65%) as a white solid. LCMS: (ESI): m/z calcd. for C₃₁H₃₇N₆O₄ 557.28 [M+H]⁺, found 557.3. ¹H NMR (400 MHz, CD₃OD) δ: 8.08 (s, 1H), 7.86 (d, J=8.8 Hz, 1H), 7.48-7.41 (m, 3H), 7.01 (d, J=3.8 Hz, 1H), 6.80 (d, J=8.8 Hz, 1H), 6.60 (d, J=3.8 Hz, 1H), 5.88 (d, J=6.3 Hz, 1H), 5.23 (br d, J=7.3 Hz, 1H), 4.98 (d, J=1.8 Hz, 1H), 2.95-2.83 (m, 2H), 2.58 (quin, J=7.0, 11.3 Hz, 2H), 2.23-2.15 (m, 1H), 2.01-1.93 (m, 1H), 1.50 (dd, J=3.9, 8.4 Hz, 1H), 1.37-1.33 (m, 1H), 1.22 (d, J=7.0 Hz, 3H), 1.19-1.13 (m, 9H), 0.94-0.89 (m, 1H).

### Example 22

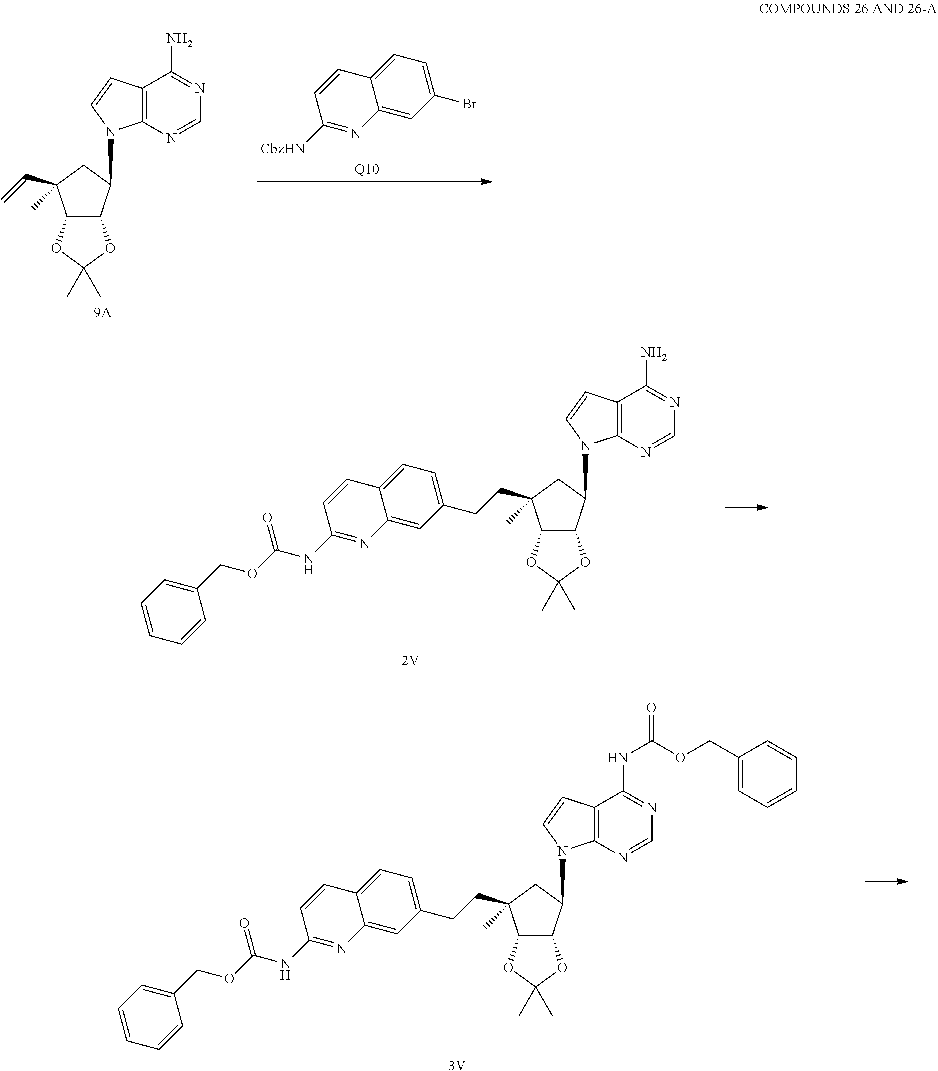

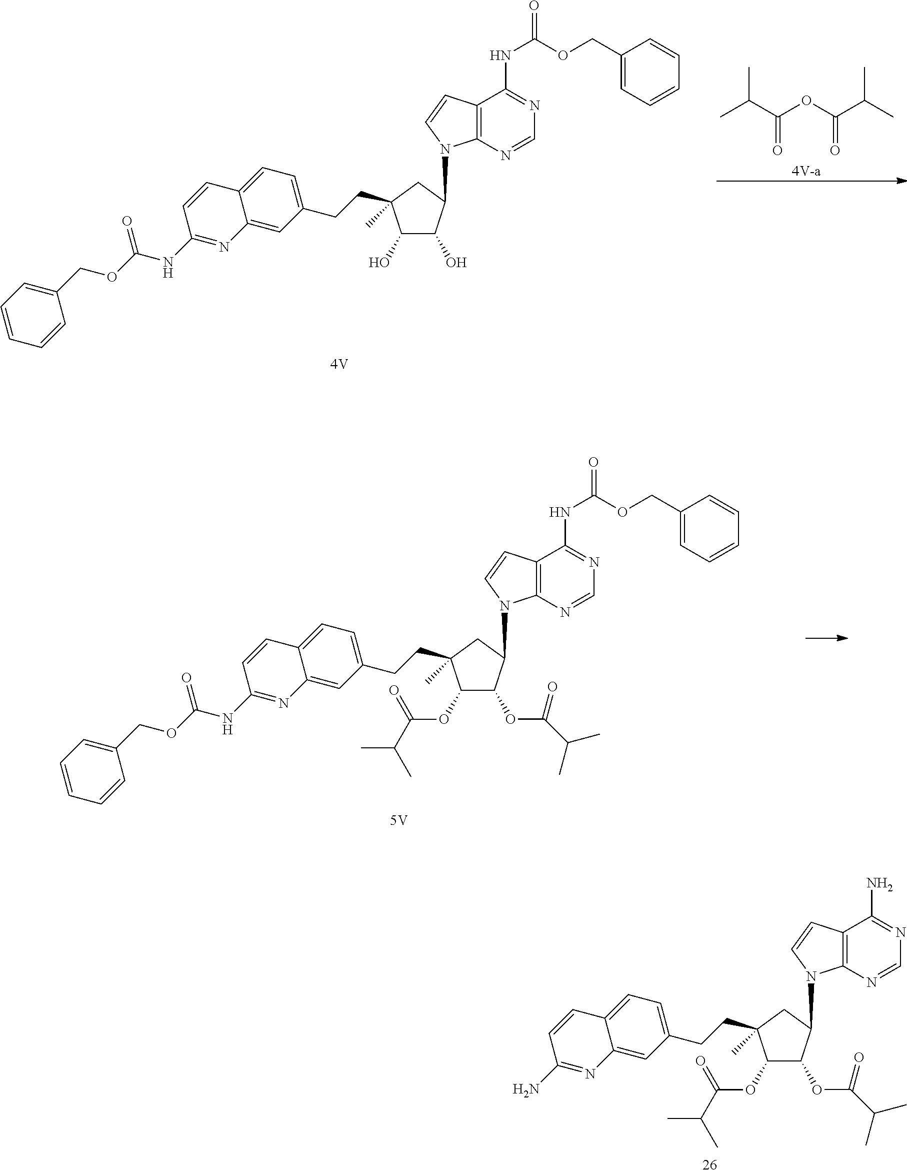

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (400 mg, 1.27 mmol) and 9-BBN dimer (769.82 mg, 3.18 mmol) in THF (8 mL) was stirred at 50° C. for 2 h under Ar and then cooled to rt. A solution of K₃PO₄ (1.35 g, 6.36 mmol) in H₂O (0.8 mL) was added. The mixture was stirred for 0.5 h. Benzyl N-(7-bromo-2-quinolyl)carbamate (Q10) (545.38 mg, 1.53 mmol) and Pd(dppf)Cl₂ (93.10 mg, 0.127 mmol) were added. The mixture was stirred at 70° C. for 12 h under Ar. The mixture was extracted with EA (2×10 mL). The combined organic layers were washed with brine (2×10 mL) and dried over Na₂SO₄. The solids were removed by filtration, and the filtrate was concentrated under reduced pressure. The residue was purified by silica gel chromatography (20%˜50% EA/PE gradient to 0%˜12% DCM/CH₃OH) to afford benzyl (7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)quinolin-2-yl)carbamate (2V) (584 mg, 0.871 mmol, 68% yield) as a yellow solid. LCMS: (ESI): m/z calcd. for C₃₄H₃₇N₆O₄ 593.28 [M+H]⁺, found 593.4.

Intermediate 3V was prepared similarly as described for 3U-A starting from 2V with the change that the reaction was stirred for 16 h to afford benzyl (7-((3aS,4R,6S,6aR)-6-(2-(2-(((benzyloxy)carbonyl)amino)quinolin-7-yl)ethyl)-2,2,6-trimethyltetrahydro-4H-cyclopenta[d][1,3]dioxol-4-yl)-7H-pyrrolo[2,3-d]pyrimidin-4-yl)carbamate (3V) (360 mg, 0.492 mmol, 56%) as a white solid. LCMS: (ESI): m/z calcd. for C₄₂H₄₃N₆O₆ 727.32 [M+H]⁺, found 727.4.

To a solution of 3V (360 mg, 492.34 μmol) in THF (4 mL) was added HCl (4 M, 2 mL). The mixture was stirred at 25° C. for 12 h. The reaction was quenched by NH₃.H₂O (1 mL) and extracted with EA (2×5 mL). The combined organic layers were washed with brine (5 mL), dried over Na₂SO₄, filtered and concentrated under reduced pressure to give benzyl (7-((1R,2S,3R,4S)-4-(2-(2-(((benzyloxy)carbonyl)amino)quinolin-7-yl)ethyl)-2,3-dihydroxy-4-methylcyclopentyl)-7H-pyrrolo[2,3-d]pyrimidin-4-yl)carbamate (4V) (328 mg, crude) as a white solid. LCMS: (ESI): m/z calcd. for C₃₉H₃₉N₆O₆ 687.29 [M+H]⁺, found 687.3.

To a mixture of 4V (328 mg, 0.478 mmol), TEA (289.98 mg, 2.87 mmol, 0.399 mL), and DMAP (5.83 mg, 0.048 mmol) in DMF (5 mL) was added 2-methylpropanoyl 2-methylpropanoate (4V-a) (226.67 mg, 1.43 mmol, 0.238 mL). The mixture was stirred at 60° C. for 3 h. The reaction was quenched by NaHCO₃ (sat., aq., 5 mL) and extracted with EA (2×5 mL). The combined organic layers were washed with brine (5 mL) and dried over Na₂SO₄. The solids were removed by filtration, and the filtrate was concentrated under reduced pressure. The residue was purified by silica gel chromatography (0˜50% EA/PE gradient) to afford (1S,2R,3S,5R)-5-(4-(((benzyloxy)carbonyl)amino)-7H-pyrrolo[2,3-d]pyrimidin-7-yl)-3-(2-(2-(((benzyloxy)carbonyl)amino)quinolin-7-yl)ethyl)-3-methylcyclopentane-1,2-diyl bis(2-methylpropanoate) (5V) (295 mg, 0.320 mmol, 67% yield) as a white solid. LCMS: (ESI): m/z calcd. for C₄₇H₅₁N₆O₈ 827.37 [M+H]⁺, found 827.4. ¹H NMR (400 MHz, CDCl₃) δ: 8.50 (s, 1H), 8.20-8.10 (m, 2H), 7.81 (br s, 2H), 7.70 (d, J=7.8 Hz, 1H), 7.62 (s, 1H), 7.44-7.34 (m, 10H), 7.30 (br dd, J=1.5, 8.3 Hz, 1H), 7.14 (d, J=3.7 Hz, 1H), 7.05 (d, J=3.7 Hz, 1H), 5.77 (dd, J=6.0, 7.8 Hz, 1H), 5.40-5.31 (m, 2H), 5.26 (s, 4H), 2.93-2.78 (m, 2H), 2.65 (quin, J=7.0 Hz, 1H), 2.41 (td, J=7.0, 14.0 Hz, 1H), 2.29-2.20 (m, 1H), 2.18-2.11 (m, 1H), 1.99 (br t, J=8.6 Hz, 2H), 1.29-1.25 (m, 9H), 1.04 (d, J=7.0 Hz, 3H), 0.99 (d, J=7.0 Hz, 3H).

Cbz deprotection was prepared similarly as described for 5U-A, starting from 5V, to afford (1S,2R,3S,5R)-5-(4-amino-7H-pyrrolo[2,3-d]pyrimidin-7-yl)-3-(2-(2-aminoquinolin-7-yl)ethyl)-3-methylcyclopentane-1,2-diyl bis(2-methylpropanoate) (26) (105 mg, 187.46 μmol, 58%) as a white solid. LCMS: (ESI): m/z calcd. for C₃₁H₃₉N₆O₄ 559.30 [M+H]⁺, found 559.5. ¹H NMR (400 MHz, CD₃OD) δ: 8.08 (s, 1H), 7.88 (d, J=9.0 Hz, 1H), 7.56 (d, J=8.0 Hz, 1H), 7.39 (s, 1H), 7.26 (d, J=3.5 Hz, 1H), 7.15 (dd, J=1.4, 8.2 Hz, 1H), 6.76 (d, J=8.8 Hz, 1H), 6.60 (d, J=3.8 Hz, 1H), 5.69 (dd, J=6.4, 7.7 Hz, 1H), 5.36 (d, J=6.5 Hz, 1H), 5.34-5.28 (m, 1H), 2.89-2.76 (m, 2H), 2.71-2.61 (m, 1H), 2.46-2.37 (m, 1H), 2.19 (d, J=9.3 Hz, 2H), 1.97 (t, J=8.5 Hz, 2H), 1.29 (s, 3H), 1.23 (dd, J=7.0, 8.8 Hz, 6H), 1.02 (d, J=6.8 Hz, 3H), 0.97 (d, J=6.8 Hz, 3H).

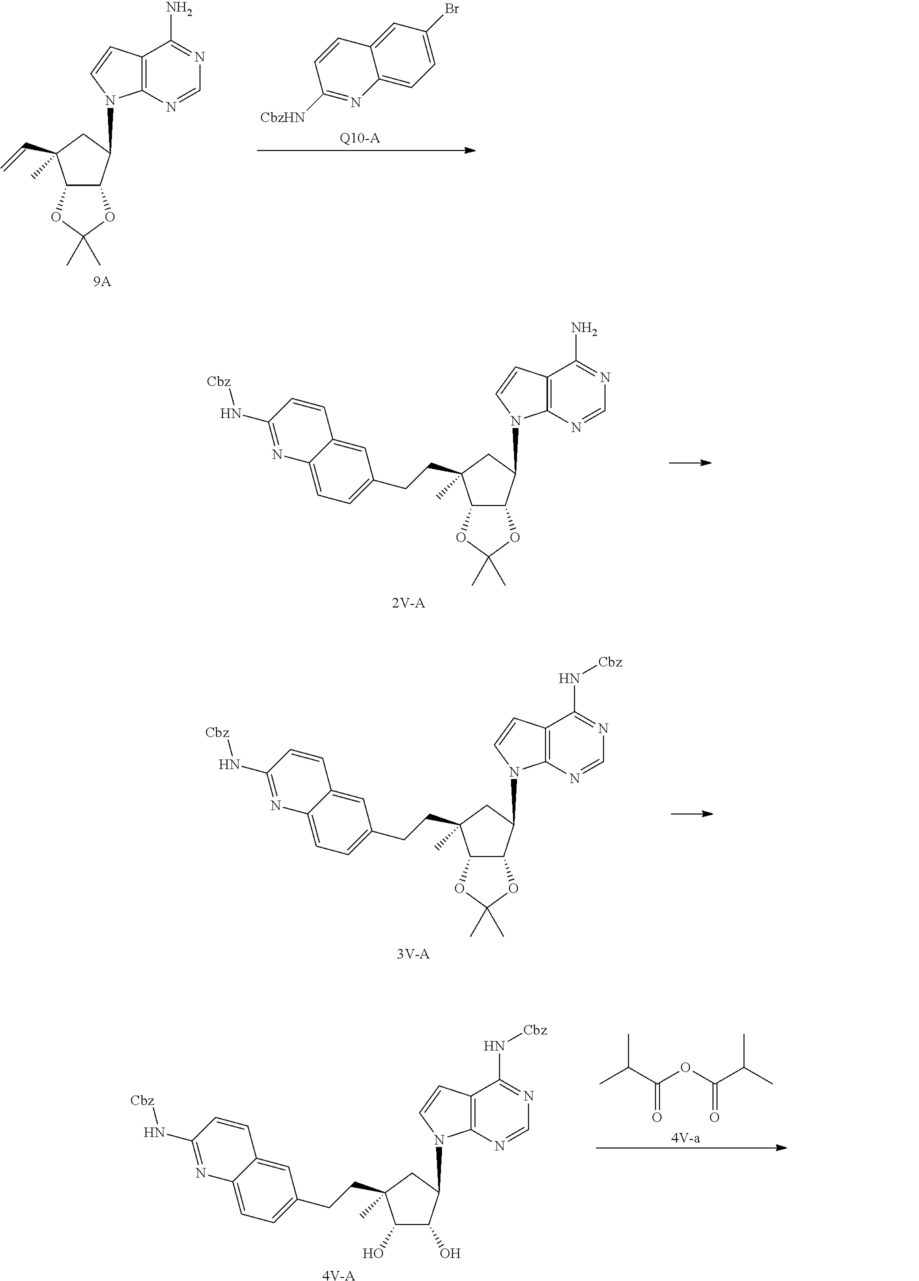

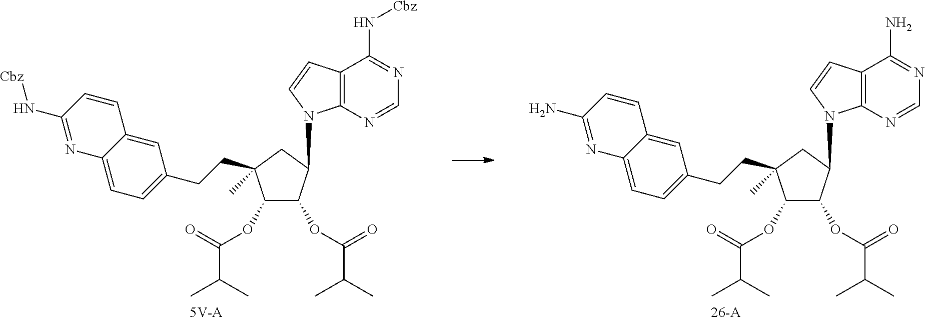

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (300 mg, 954.3 μmol, 1 eq.) and 9-BBN dimer (577.4 mg, 2.39 mmol, 2.5 eq.) in THF (8 mL) was stirred at 50° C. for 2 h under Ar and then cooled to rt. A solution of K₃PO₄ (1.01 g, 4.77 mmol, 5 eq.) in H₂O (0.8 mL) was added. The mixture was stirred for 0.5 h. Benzyl N-(6-bromo-2-quinolyl)carbamate (Q10-a) (409.0 mg, 1.15 mmol, 1.2 eq.) and Pd(dppf)Cl₂ (69.8 mg, 95.4 μmol, 0.1 eq.) were added. The mixture was stirred at 70° C. for 12 h under Ar. The reaction progress was monitored by TLC (DCM:MeOH=20:1). Upon completion, the mixture was extracted with EA (2×5 mL). The combined organic layers were washed with brine (2×5 mL), dried over Na₂SO₄, filtered and concentrated under reduced pressure to give a residue. The residue was purified by flash silica gel chromatography (ISCO®; 20 g SepaFlash® Silica Flash Column, Eluent of 10%˜50% EA:PE gradient to 0%˜10% DCM/MeOH @ 35 mL/min) to afford 2V-A (312 mg, 431.7 μmol, 45% yield) as a yellow solid. LCMS: (ESI): m/z calcd. for C₃₄H₃₇N₆O₄ 593.28 [M+H]⁺, found 593.3.

To a solution of 2V-A (312 mg, 431.7 μmol, 1 eq.) in DCM (8 mL) were added 1-methylimidazole (283.5 mg, 3.45 mmol, 275, 8 eq.) and CbzCl (294.6 mg, 1.73 mmol, 245 μL, 4 eq.) at 0° C. The mixture was stirred at 25° C. for 16 h. The reaction was quenched by addition of sat. NaHCO₃ solution (5 mL) and then extracted with DCM (3×5 mL). The combined organic layers were washed with brine, dried over Na₂SO₄, filtered and concentrated under reduced pressure to give a residue. The residue was purified by flash silica gel chromatography (ISCO®; 20 g SepaFlash® Silica Flash Column, Eluent of 10%˜50% EA:PE gradient to 0%˜15% DCM/MeOH @ 35 mL/min) to afford 3V-A (178 mg, 233.9 μmol, 54% yield) as a white solid. LCMS: (ESI): m/z calcd. for C₄₂H₄₃N₆O₆ 727.32 [M+H]⁺, found 727.3.

To a solution of 3V-A (178 mg, 233.88 μmol, 1 eq.) in THF (2 mL) was added HCl (4 M, 1 mL). The mixture was stirred at 25° C. for 12 h. The reaction progress was monitored by TLC (DCM:MeOH=20:1). Upon completion, the reaction was quenched by NH₃.H₂O solution (1 mL) and extracted with EA (2×5 mL). The combined organic layers were washed with brine (2×5 mL), dried over Na₂SO₄, filtered and concentrated under reduced pressure to give 4V-A (161 mg, crude) as a white solid. LCMS: (ESI): m/z calcd. for C₃₉H₃₉N₆O₆ 687.29 [M+H]⁺, found 687.3.

To a mixture of 4V-A (200 mg, 291.2 μmol, 1 eq.), TEA (176.8 mg, 1.75 mmol, 243 μL, 6 eq.) and DMAP (3.6 mg, 29.1 μmol, 0.1 eq.) in DMF (5 mL) was added 2-methylpropanoyl 2-methylpropanoate (4V-a) (138.2 mg, 873.7 μmol, 145 μL, 3 eq.). The mixture was stirred at 60° C. for 3 h. The reaction progress was monitored by TLC (PE:EA=1:1). Upon completion, the reaction was quenched by sat.NaHCO₃ solution (5 mL) and extracted with EA (2×5 mL). The combined organic layers were washed with brine (2×5 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜50% EA:PE gradient @ 30 mL/min) to afford 5V-A (196 mg, 208.6 μmol, 71% yield) as a white solid. LCMS: (ESI): m/z calcd. for C₄₇H₅₁N₆O₈ 827.37 [M+H]⁺, found 827.6.

To a solution of 5V-A (213 mg, 257.6 μmol, 1 eq.) in EtOH (5 mL) and THF (2 mL) was added Pd/C (100 mg, 10% wt). The mixture was degassed under reduced pressure and purged with H₂ (3×). The mixture was then stirred under H₂ atmosphere (15 psi) at 25° C. for 20 h. The reaction progress was monitored by TLC (DCM:MeOH=10:1). Upon completion, the mixture was filtered through a Celite pad to remove the Pd/C. The filtrate was concentrated under reduced pressure to afford a residue. The residue was purified by prep-HPLC (column: Welch Xtimate C18 150*25 mm*5 um; mobile phase: [water (10 mM NH₄HCO₃)-ACN]; B %: 45%-75%, 8.5 min) to afford Compound 26-A (73 mg, 130.5 μmol, 50.7% yield, 99.9% purity) as a white solid. LCMS: (ESI): m/z calcd. for C₃₁H₃₉N₆O₄ 558.30 [M+H]⁺, found 558.3. ¹H NMR (400 MHz, CD₃OD) δ: 8.07 (s, 1H), 7.89 (d, J=8.8 Hz, 1H), 7.51-7.43 (m, 3H), 7.25 (d, J=3.8 Hz, 1H), 6.80 (d, J=8.8 Hz, 1H), 6.60 (d, J=3.8 Hz, 1H), 5.70 (dd, J=6.4, 7.9 Hz, 1H), 5.38-5.28 (m, 2H), 2.88-2.74 (m, 2H), 2.67 (spt, J=7.0 Hz, 1H), 2.48-2.36 (m, 1H), 2.25-2.13 (m, 2H), 1.96 (dd, J=7.8, 9.3 Hz, 2H), 1.29 (s, 3H), 1.23 (dd, J=7.0, 8.3 Hz, 6H), 1.02 (d, J=6.8 Hz, 3H), 0.97 (d, J=7.0 Hz, 3H).

### Example 23

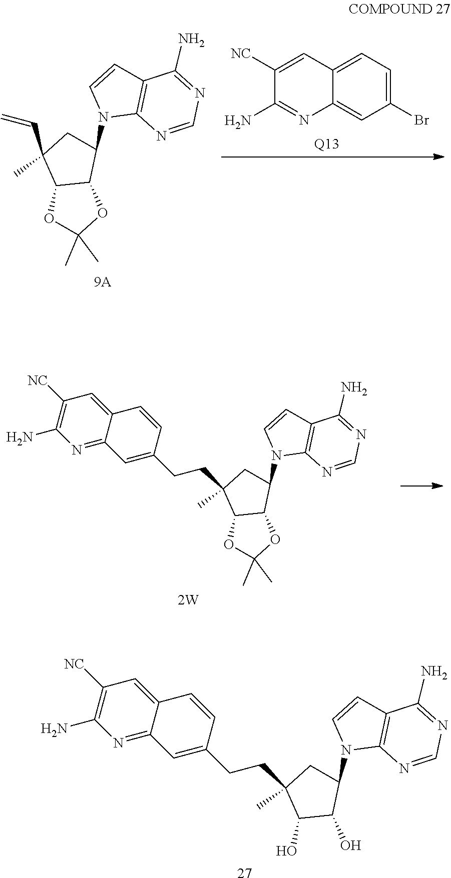

To a mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (150 mg, 477.1 μmol, 1 eq.) in THF (5 mL) was added 9-BBN dimer (254.0 mg, 1.05 mmol, 2.2 eq.) at 20° C. The mixture was stirred at 50° C. for 1.5 h under Ar, and then cooled to 20° C. A solution of K₃PO₄ (506.4 mg, 2.39 mmol, 5 eq.) in H₂O (1 mL) was added, and the mixture was stirred for 30 min. 2-amino-7-bromo-quinoline-3-carbonitrile (142.0 mg, 572.6 μmol, 1.2 eq.) and Pd(dppf)Cl₂ (34.9 mg, 47.7 μmol, 0.1 eq.) were added to the mixture at 20° C., and the mixture was degassed for several times. The mixture was then stirred at 70° C. for 16 h under Ar. The reaction progress was monitored by LCMS. Upon completion, the mixture was partitioned between EA (20 mL) and brine (10 mL). The organic phase was separated, and the aqueous phase was extracted with EA (3×20 mL). The organics were combined and dried over Na₂SO₄, filtered and concentrated under reduced pressure to give a residue. The residue was purified by column chromatography (SiO₂, PE:EA:EtOH=20:20:1 to 30:70:3) to provide 2W (105 mg, 209.1 μmol, 43.8% yield, 96.3% purity) as a yellow solid. LCMS: (ESI): m/z calcd. for C₂₇H₃₀N₇O₂ 484.25 [M+H]⁺, found 484.2.

To a solution of 2W (105 mg, 209.1 μmol, 43.8% yield, 96.3% purity, 1 eq.) in THF (6 mL) was added HCl (4 M, 3 mL), and the mixture was stirred at 20° C. for 12 h. The mixture was concentrated under reduced pressure give a residue. The residue was purified by prep-HPLC (HCl condition, column: Phenomenex Gemini-NX 150*30 mm*5 um; mobile phase: [water (0.05% HCl)-ACN]; B %: 3%-30%) followed by prep-HPLC (basic condition, Column: YMC Triart C18 150*25 mm*5 um, Condition: water (10 mM NH₄HCO₃)-ACN, B %: 24%-54%) to afford Compound 27 (54 mg, 120.8 μmol, 57.7% yield, 99.2% purity) as a white solid. LCMS: (ESI): m/z calcd. for C₂₄H₂₆N₇O₂ 444.21 [M+H]⁺, found 444.3. ¹H NMR (400 MHz, CD₃OD) δ: 8.46 (s, 1H), 8.08 (s, 1H), 7.65 (d, J=8.3 Hz, 1H), 7.43 (s, 1H), 7.18-7.28 (m, 2H), 6.60 (d, J=3.4 Hz, 1H), 4.95-5.06 (m, 1H), 4.53 (t, J=6.8 Hz, 1H), 3.93 (d, J=6.4 Hz, 1H), 2.72-2.95 (m, 2H), 2.03-2.13 (m, 1H), 1.81-2.00 (m, 3H), 1.24 (s, 3H).

### Example 24

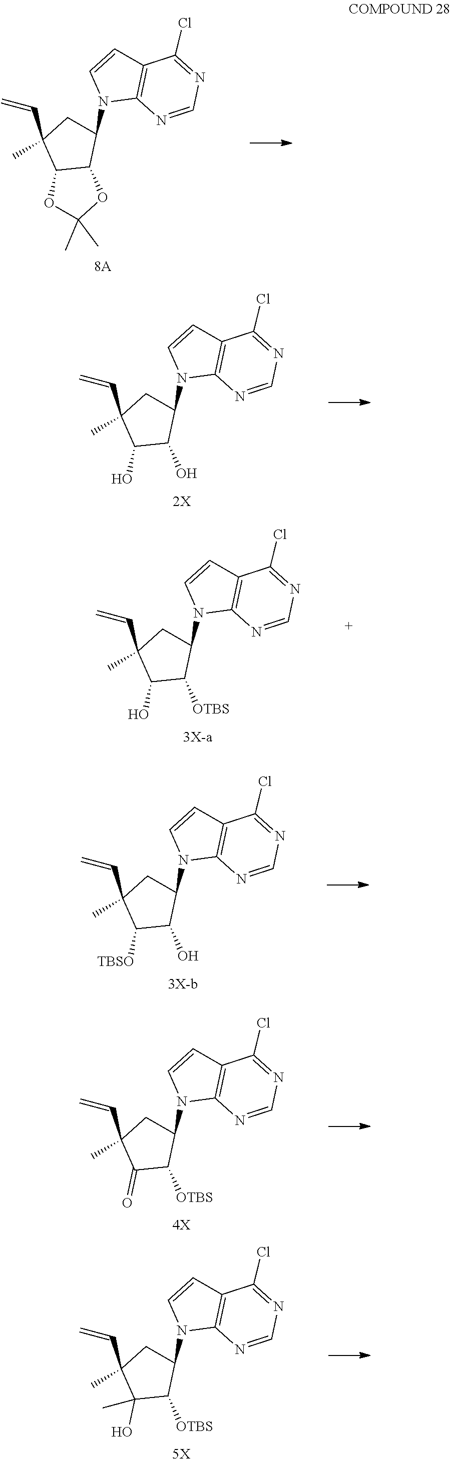

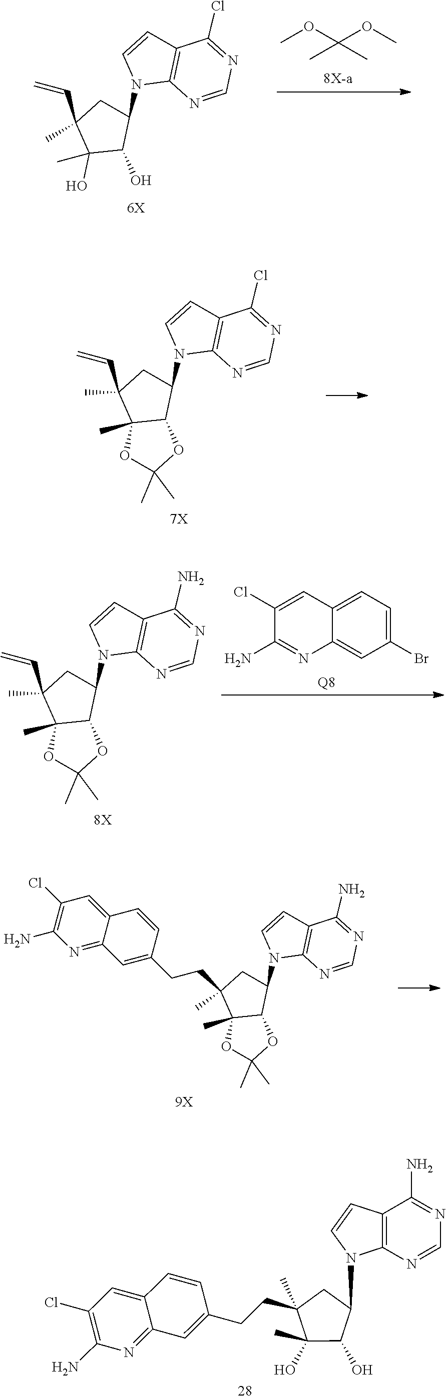

To a solution of 8A (1 g, 3.00 mmol, 1 eq.) in THF (10 mL) was added HCl (4 M, 5 mL, 6.68 eq.). The mixture was stirred at 20° C. for 16 h. The reaction progress was monitored by LCMS. Upon completion, the mixture was concentrated under reduced pressure to give a residue and then adjusted to pH=7 with NaHCO₃ solution before extraction with DCM (2×10 mL). The combined organic layers were dried over Na₂SO₄, filtered and concentrated under reduced pressure to give (1S,2R,3R,5R)-5-(4-chloropyrrolo[2,3-d]pyrimidin-7-yl)-3-methyl-3-vinyl-cyclopentane-1,2-diol (2X) (790 mg, 2.66 mmol, 88.9% yield, 99% purity) as a white solid. LCMS: (ESI): m/z calcd. for C₁₄H₁₇ClN₃O₂ 294.09 [M+H]⁺, found 293.9.

To a solution of 2X (790 mg, 2.69 mmol, 1 eq.) in DCM (8 mL) were added 2,4,6-trimethylpyridine (651.8 mg, 5.38 mmol, 711 μL, 2 eq.), AgNO₃ (685.3 mg, 4.03 mmol, 1.5 eq.) and TBSCl (445.9 mg, 2.96 mmol, 362 μL, 1.1 eq.). The mixture was stirred at 20° C. for 5 h, filtered and concentrated under reduced pressure to give a residue. The residue was purified by flash silica gel chromatography (ISCO®; 20 g SepaFlash® Silica Flash Column, Eluent of 0˜17% EA:PE ether gradient @ 35 mL/min) to give a mixture of 3X-a and 3X-b (690 mg, 3a:3b=2.5:1).

To a solution of 3X-a and 3X-b (850 mg, 2.08 mmol, 1 eq.) in DCM (10 mL) was added PCC (898.1 mg, 4.17 mmol, 2 eq.) and 4 A molecular sieves (800 mg). The mixture was stirred at 20° C. for 2 h. The reaction progress was monitored by LCMS. Upon completion, the mixture was filtered and concentrated under reduced pressure to give a residue. The residue was purified by flash silica gel chromatography (ISCO®; 12 g SepaFlash® Silica Flash Column, Eluent of 0˜25% EA:PE gradient @ 30 mL/min), and then by prep-HPLC (neutral condition: column: YMC Triart C18 150*25 mm*5 um; mobile phase: [water (10 mM NH₄HCO₃)-ACN]; B %: 77%-100%, 9.5 min) to give (2R,4R,5S)-5-[tert-butyl(dimethyl)silyl]oxy-4-(4-chloropyrrolo[2,3-d]pyrimidin-7-yl)-2-methyl-2-vinylcyclopentanone (4X) (540 mg, 1.32 mmol, 63.2% yield, 99% purity) as a white solid. LCMS: (ESI): m/z calcd. for C₂₀H₂₉ClN₃O₂Si 406.16 [M+H]⁺, found 406.3.

To a solution of 4X (540 mg, 1.33 mmol, 1 eq.) in THF (6 mL) was added bromo(methyl)magnesium (3 M, 1.33 mL, 3 eq.) at −78° C. The mixture was stirred at 0° C. for 6 h. The reaction progress was monitored by LCMS. Upon completion, the reaction was quenched by NH₄Cl solution (5 mL), diluted with water (5 mL) and extracted with DCM (2×5 mL). The combined organic layers were washed with brine (2×5 mL), dried over Na₂SO₄, filtered and concentrated under reduced pressure to give (1R,2R,4R,5S)-5-[tert-butyl(dimethyl)silyl]oxy-4-(4-chloropyrrolo[2,3-d]pyrimidin-7-yl)-1,2-dimethyl-2-vinylcyclopentanol (5X) (480 mg, crude) as a colorless oil.

To a solution of 5X (480 mg, crude, 1 eq.) in THF (10 mL) was added HCl (4 M, 5 mL). The mixture was stirred at 20° C. for 36 h. The reaction progress was monitored by LCMS. Upon completion, the mixture was concentrated under reduced pressure to give a residue. The residue was then adjusted to pH=7 with NaHCO₃ solution and extracted with DCM (2×10 mL). The combined organic layers were dried over Na₂SO₄, filtered and concentrated under reduced pressure to give (1R,2S,3R,5R)-3-(4-chloropyrrolo[2,3-d]pyrimidin-7-yl)-1,5-dimethyl-5-vinyl-cyclopentane-1,2-diol (6X) (380 mg, crude) as a purple solid.

To a solution of 6X (380 mg, crude, 1 eq.) and 2,2-dimethoxypropane (385.76 mg, 3.70 mmol, 454 μL, 3 eq.) in acetone (10 mL) was added TsOH.H₂O (7.0 mg, 37.0 μmol, 0.03 eq.) at 0° C. The mixture was stirred at 25° C. for 2 h. The reaction progress was monitored by LCMS. Upon completion, the reaction was quenched by the addition NaHCO₃ solution (10 mL) and extracted with DCM (2×10 mL). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜8% EA:PE gradient @ 30 mL/min) to give 7X (280 mg, 780.8 μmol, 63% yield, 97% purity) as a colorless oil. LCMS: (ESI): m/z calcd. for C₁₈H₂₃ClN₃O₂ 348.14 [M+H]⁺, found 348.1.

To a mixture of NH₃.H₂O (4.55 g, 32.46 mmol, 5 mL, 25%, 45.16 eq.) and dioxane (5 mL) was added 7X (250 mg, 718.7 μmol, 1 eq.). The mixture was heated in a sealed tube at 100° C. and stirred for 48 h. The reaction progress was monitored by LCMS. Upon completion, the mixture was extracted with DCM (2×5 mL). The combined organic layers were washed with brine (2×5 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜75% EA/PE @ 30 mL/min) to give 7-[(3aR,4R,6R,6aS)-2,2,3a,4-tetramethyl-4-vinyl-6,6a-dihydro-5H-cyclopenta[d][1,3]dioxol-6-yl]pyrrolo[2,3-d]pyrimidin-4-amine (8X) (158 mg, 476.3 μmol, 66.3% yield, 99% purity) as a colorless oil. LCMS: (ESI): m/z calcd. for C₁₈H₂₅N₄O₂ 329.19 [M+H]⁺, found 329.3.

To a solution of 8X (158 mg, 481.1 μmol, 1 eq.) in THF (10 mL) was added 9-BBN dimer (465.8 mg, 1.92 mmol, 4 eq.) at 25° C. The mixture was stirred at 50° C. for 1 h. The mixture was then cooled to 25° C. and a solution of K₃PO₄ (1.02 g, 4.81 mmol, 10 eq.) in H₂O (4 mL) was added. The mixture was stirred for 30 min, and then 7-bromo-3-chloro-quinolin-2-amine (161.1 mg, 625.4 μmol, 1.3 eq.) and Pd(dppf)Cl₂ (35.2 mg, 48.1 μmol, 0.1 eq.) were added at 25° C. The mixture was stirred at 70° C. for 15 h. The reaction progress was monitored by LCMS. Upon completion, the reaction mixture was filtered. The filtrate was diluted with brine (10 mL) and extracted with DCM (2×10 mL). The combined organic layers dried over Na₂SO₄, filtered and concentrated under reduced pressure to give a residue. The residue was purified by column chromatography (SiO₂, PE:EA=100:1 to 0:1, DCM:MeOH=50:1 to 10:1) to give 7-[2-[(3aR,4S,6R,6aS)-6-(4-aminopyrrolo[2,3-d]pyrimidin-7-yl)-2,2,3a,4-tetramethyl-6,6a-dihydro-5Hcyclopenta[d][1,3]dioxol-4-yl]ethyl]-3-chloro-quinolin-2-amine (9X) (180 mg, 284.0 μmol, 59.0% yield, 80% purity) as a yellow solid. LCMS: (ESI): m/z calcd. for C₂₇H₃₂ClN₆O₂ 507.22 [M+H]⁺, found 507.2.

To a solution of 9X (180 mg, 284.01 μmol, 80% purity, 1 eq.) in THF (5 mL) was added HCl (4 M, 2.00 mL). The mixture was stirred at 20° C. for 12 h. The reaction progress was monitored by LCMS. Upon completion, the mixture was concentrated under reduced pressure to give a residue. The residue was purified by prep-HPLC (basic condition: column: Phenomenex Gemini-NX C18 75*30 mm*3 um; mobile phase: [water (0.04% NH₃H₂O+10 mM NH₄HCO₃)-ACN]; B %: 30%-60%, 7 min) to give Compound 28 (63 mg, 132.2 μmol, 46.6% yield, 98% purity) as a white solid. LCMS: (ESI): m/z calcd. for C₂₄H₂₈ClN₆O₂ 467.19 [M+H]⁺, found 467.2. ¹H NMR (400 MHz, CD₃OD) δ: 8.07 (d, J=2.4 Hz, 2H), 7.58 (d, J=8.3 Hz, 1H), 7.45 (s, 1H), 7.20 (dd, J=1.2, 8.3 Hz, 1H), 7.17 (d, J=3.7 Hz, 1H), 6.59 (d, J=3.4 Hz, 1H), 4.96 (q, J=8.9 Hz, 1H), 4.48 (d, J=9.0 Hz, 1H), 2.87-2.71 (m, 2H), 2.23-2.13 (m, 2H), 2.00 (dt, J=4.9, 12.2 Hz, 1H), 1.79 (dt, J=5.6, 12.3 Hz, 1H), 1.23 (s, 3H), 1.20 (s, 3H).

### Example 25

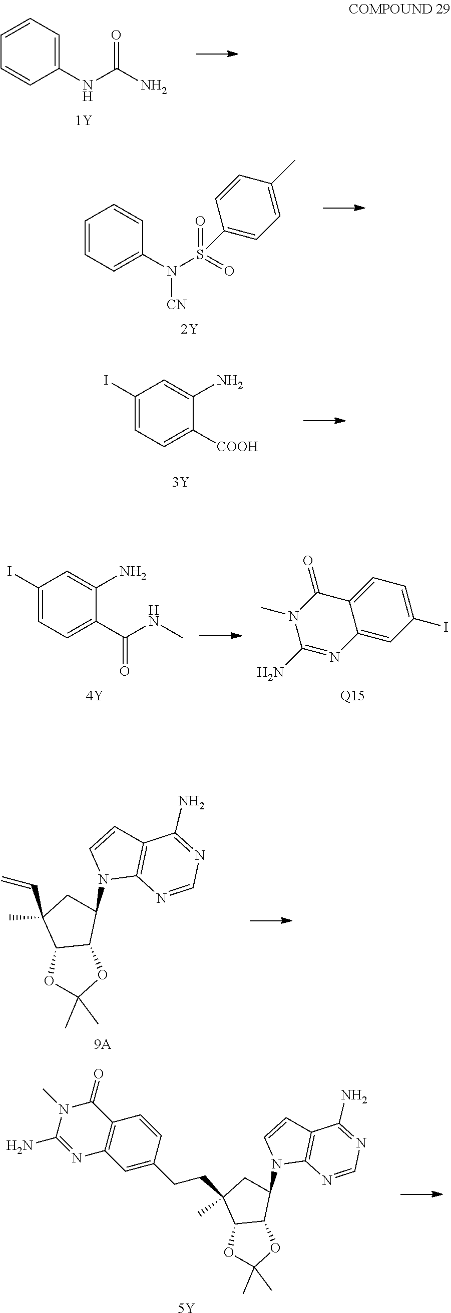

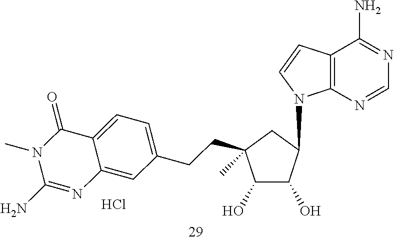 [SMILES: Cn1c(N)nc2cc(CC[C@@]3(C)C[C@@H](n4ccc5c(N)ncnc54)[C@H](O)[C@@H]3O)ccc2c1=O ; Cl ; "29"]

To a solution of phenylurea (1Y) (1 g, 7.3 mmol, 1 eq.) in pyridine (5 mL) was added dropwise toluenesulfonyl chloride (4.90 g, 25.7 mmol, 3.71 mL, 3.5 eq.) at 20° C. After addition, the mixture was stirred at 20° C. for 15 min. The reaction progress was monitored by TLC (PE:EA=1:1). Upon completion, the reaction was quenched by addition of ice-cooled water (30 mL) at 20° C. The precipitate formed during stirring was filtered and washed with water. The solid was collected by filtration to give a crude product. The crude product was triturated with EtOH (5 mL) at 20° C. for 30 min and then filtered to give 2Y (800 mg, 2.9 mmol, 40% yield) as a white solid. ¹H NMR (400 MHz, CDCl₃) δ: 7.64 (d, J=8.3 Hz, 2H), 7.33-7.45 (m, 5H), 7.20 (d, J=7.5 Hz, 2H), 2.48 (s, 3H).

To a solution of 2-amino-4-iodo-benzoic acid (3Y) (1 g, 3.8 mmol, 1 eq.), methanamine hydrochloride (308 mg, 4.6 mmol, 1.20 eq.) and DIEA (1.47 g, 11.4 mmol, 1.99 mL, 3 eq.) in DCM (12 mL) were added EDCI (729 mg, 3.8 mmol, 1 eq.) and HOBt (616 mg, 4.7 mmol, 1.2 eq.). The mixture was stirred at 20° C. for 3.5 h. The reaction progress was monitored by LCMS. Upon completion, the reaction was quenched by addition of water (15 mL). The mixture was then partitioned between EA (20 mL) and a sat. Na₂CO₃ solution (30 mL). The organic phase was separated, and the aqueous phase washed with EA (3×20 mL). The combined organic layers were combined, dried over Na₂SO₄, filtered and concentrated under reduced pressure to give a residue. The residue was purified by flash silica gel chromatography (ISCO®; 20 g SepaFlash® Silica Flash Column, Eluent of 0˜50% EA:PE gradient @ 30 mL/min) to give 2-amino-4-iodo-N-methyl-benzamide (4Y) (1 g, 3.6 mmol, 95% yield, 100% purity) as a white solid. LCMS: (ESI): m/z calcd. for C₈H₁₀IN₂O 276.98 [M+H]⁺, found 276.9.

To a solution of 4Y (300 mg, 1.1 mmol, 1 eq.) in dioxane (8 mL) were added N-cyano-4-methyl-N-phenyl-benzenesulfonamide (296 mg, 1.1 mmol, 1 eq.) and LiHMDS (1 M, 3.3 mL, 3 eq.) at 20° C. The mixture was stirred at 100° C. for 1 h. The reaction progress was monitored by LCMS. Upon completion, the reaction was quenched by water (10 mL). The aqueous phase washed with EA (3×20 mL). The organic layers were combined, washed with brine (20 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜100% EA:PE gradient @ 35 mL/min). Q15 (225 mg, 729 μmol, 67% yield, 98% purity) was obtained as a white solid. LCMS: (ESI): m/z calcd. for C₉H₉IN₃O 301.97 [M+H]⁺, found 301.9.

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (150 mg, 477 μmol, 1 eq.) and 9-BBN dimer (289 mg, 1.2 mmol, 2.5 eq.) in THF (5 mL) was stirred at 50° C. for 2 h under Ar. The mixture was cooled to 20° C. and then a solution of K₃PO₄ (506 mg, 2.4 mmol, 5 eq.) in H₂O (1 mL) was added. The mixture was then stirred for 30 min. Compound Q15 (172 mg, 573 μmol, 98% purity, 1.2 eq.) and Pd(dppf)Cl₂ (35 mg, 48 μmol, 0.1 eq.) were added. The mixture was stirred at 70° C. for 16 h under Ar. The reaction progress was monitored by LCMS. Upon completion, the mixture was partitioned between EA (20 mL) and brine (10 mL). The organic phase was separated, and the aqueous phase was extracted with EA (3×20 mL). The separated organic layers were combined, dried over Na₂SO₄, filtered and concentrated under reduced pressure to give a residue. The residue was purified by column chromatography (SiO₂, DCM:MeOH=20:1 to 100:7). Compound 5Y (145 mg, 277 μmol, 58% yield, 93% purity) was obtained as a yellow gum. LCMS: (ESI): m/z calcd. for C₂₆H₃₂N₇O₃ 490.25 [M+H]⁺, found 490.2.

To a solution of 5Y (145 mg, 277 μmol, 93% purity, 1 eq.) in THF (6 mL) was added HCl (4 M, 3 mL). The mixture was stirred at 20° C. for 12 h. The reaction progress was monitored by LCMS. Upon completion, the mixture was concentrated under reduced pressure to remove solvent to give a residue. The residue was purified by prep-HPLC (HCl condition: Column: Venusil ASB Phenyl 150*30 mm*5 um, Condition: water (0.05% HCl)-ACN, B %: 10%-40%) to give Compound 29 as a hydrochloride salt (white solid, 110 mg, 207 μmol, 75% yield, 98% purity). LCMS: (ESI): m/z calcd. for C₂₃H₂₈N₇O₃ 450.22 [M+H]⁺, found 450.2. ¹H NMR (400 MHz, CD₃OD) δ: 8.25 (s, 1H), 8.09 (d, J=8.1 Hz, 1H), 7.57 (d, J=3.7 Hz, 1H), 7.37 (d, J=8.1 Hz, 1H), 7.33 (s, 1H), 6.92 (d, J=3.4 Hz, 1H), 5.06-5.16 (m, 1H), 4.55 (dd, J=7.5, 6.5 Hz, 1H), 3.95 (d, J=6.1 Hz, 1H), 3.55 (s, 3H), 2.75-2.97 (m, 2H), 1.96-2.13 (m, 2H), 1.79-1.95 (m, 2H), 1.23 (s, 3H).

### Example 26

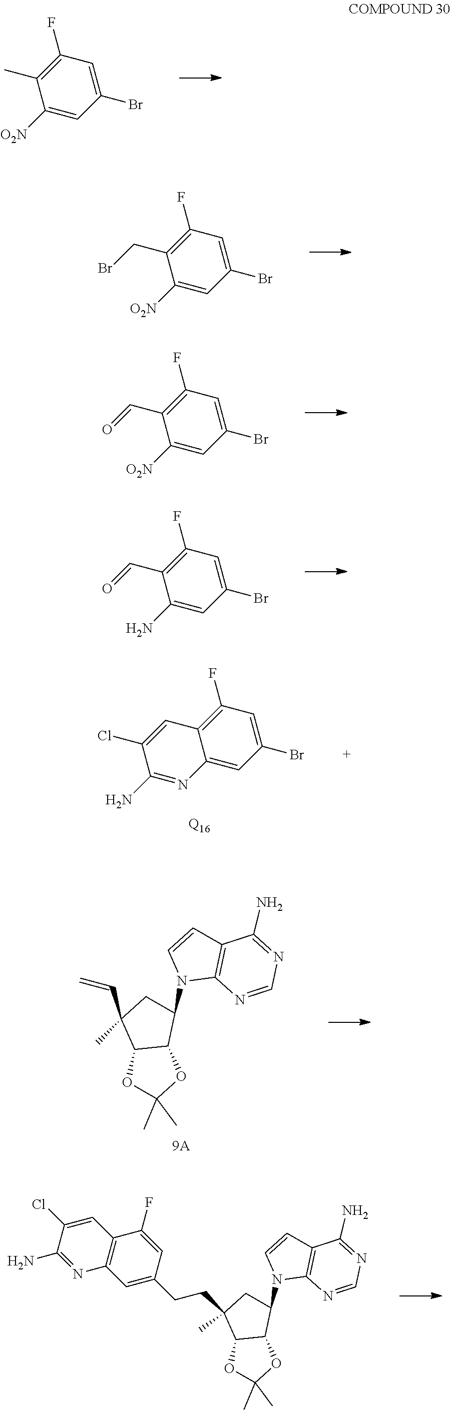

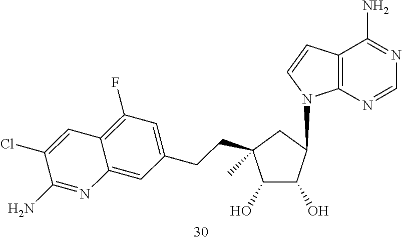 [SMILES: C[C@]1(CCc2cc(F)c3cc(Cl)c(N)nc3c2)C[C@@H](n2ccc3c(N)ncnc32)[C@H](O)[C@@H]1O ; "30"]

NBS (8.03 g, 45.12 mmol) and BPO (1.30 g, 3.76 mmol, 70% purity) were added to a solution of 5-bromo-1-fluoro-2-methyl-3-nitrobenzene (8.8 g, 37.60 mmol) in CCl₄ (130 mL) at 80° C. The mixture was stirred at 80° C. for 12 h. The mixture was extracted with EA (3×100 mL). The combined organic layers were washed with brine (2×100 mL) and dried over Na₂SO₄. The solids were removed by filtration, and the filtrate was concentrated under reduced pressure. The residue was purified by silica gel chromatography (0˜2% PE/EA gradient) to afford 5-bromo-2-(bromomethyl)-1-fluoro-3-nitro-benzene (12.4 g, 31.70 mmol, 84% yield) as a colorless oil. ¹H NMR (400 MHz, CDCl₃) δ 8.02 (s, 1H), 7.58 (dd, J=1.7, 8.6 Hz, 1H), 4.76 (d, J=1.5 Hz, 2H).

4-methyl-4-oxido-morpholin-4-ium (8.91 g, 76.08 mmol, 8.0 mL) was added to a mixture of 5-bromo-2-(bromomethyl)-1-fluoro-3-nitro-benzene (12.4 g, 31.7 mmol) and 4 Å molecular sieves (20 g) in MeCN (130 mL). The mixture was stirred at 25° C. for 3 h. The mixture was extracted with EA (3×50 mL). The combined organic layers were washed with water, 1M HCl and brine (2×50 mL) and dried over Na₂SO₄. The solids were removed by filtration, and the filtrate was concentrated under reduced pressure. The residue was purified by silica gel chromatography (0˜7% PE/EA gradient) to afford 4-bromo-2-fluoro-6-nitro-benzaldehyde (6.49 g, 26.17 mmol, 82%) as a white solid. ¹H NMR (400 MHz, CDCl₃) δ 10.24 (s, 1H), 8.02-7.98 (m, 1H), 7.68 (dd, J=1.8, 8.6 Hz, 1H).

Fe powder (14.61 g, 261.69 mmol) was added to a solution of 4-bromo-2-fluoro-6-nitro-benzaldehyde (6.49 g, 26.17 mmol) in EtOH (30 mL) and AcOH (30 mL) at 0° C. The mixture was stirred at 25° C. for 3 h, then diluted with EA (100 mL). The reaction was neutralized with NaHCO₃ (sat., aq., 300 mL). The mixture was filtered through a Celite pad. The separated organic layer was washed with brine (3×100 mL) and dried over Na₂SO₄. The solids were removed by filtration, and the filtrate was concentrated under reduced pressure to afford 2-amino-4-bromo-6-fluoro-benzaldehyde (5.67 g, crude) as a light green solid. LCMS: (ESI): m/z calcd. for C₇H₆BrFNO 217.95 [M+H]⁺, found 217.8.

To a solution of 2-amino-4-bromo-6-fluoro-benzaldehyde (3 g, 13.76 mmol) and 2,2,2-trichloroacetonitrile (2.19 g, 15.14 mmol, 1.52 mL) in THF (40 mL) was added Fe (7.68 g, 137.60 mmol). The mixture was diluted with EA (20 mL) and filtered to give a filtrate. The mixture was then extracted with EA (3×30 mL). The combined organic layers were washed with brine (3×50 mL), dried over Na₂SO₄, filtered and concentrated under reduced pressure. The residue was purified by silica gel chromatography (0˜12% EA/PE gradient) to give a solid, that was suspended in PE:EA (10:1, 20 mL) and stirred at 25° C. for 1 h. The solid was collected by filtration and dried under reduced pressure to afford 7-bromo-3-chloro-5-fluoro-quinolin-2-amine (Q16) (1.9 g, 6.90 mmol, 45% yield) as a yellow solid. LCMS: (ESI): m/z calcd. for C₉H₆BrClFN₂, 276.93 [M+H]⁺, found 276.7.

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (150 mg, 0.477 mmol) and 9-BBN dimer (288.68 mg, 1.19 mmol) in THF (5 mL) was stirred at 50° C. for 2 h under Ar and then cooled to rt. A solution of K₃PO₄ (506.39 mg, 2.39 mmol) in H₂O (0.5 mL) was added. The mixture was stirred for 0.5 h. Compound Q16 (197.18 mg, 715.69 μmol) and Pd(dppf)Cl₂ (34.91 mg, 47.71 μmol) were added. The mixture was stirred at 70° C. for 12 h under Ar. The mixture was diluted with EA (2 mL) and extracted with EA (2×5 mL). The combined organic layers were washed with brine (3×5 mL), dried over Na₂SO₄, filtered and concentrated under reduced pressure. The residue was purified by silica column chromatography (PE:EA=50:1 to 1:2 to DCM:MeOH=100:1 to 20:1) to afford 7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-3-chloro-5-fluoroquinolin-2-amine (193 mg, 339.9 μmol, 71%) as a yellow solid. LCMS: (ESI): m/z calcd. for C₂₆H₂₉ClFN₆O₂ 511.19 [M+H]⁺, found 511.3.

To a solution of 7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-3-chloro-5-fluoroquinolin-2-amine (193 mg, 0.340 mmol) in THF (4 mL) was added HCl (4 M, 2 mL). The mixture was stirred at 25° C. for 12 h. The mixture was filtered and concentrated under reduced pressure. The solid was added to MeCN:H₂O (10:1, 3×10 mL) and stirred at 60° C. for 1 h. The mixture was filtered, and the collected solid was dried under reduced pressure to afford (1S,2R,3S,5R)-3-(2-(2-amino-3-chloro-5-fluoroquinolin-7-yl)ethyl)-5-(4-amino-7H-pyrrolo[2,3-d]pyrimidin-7-yl)-3-methylcyclopentane-1,2-diol (30) as a hydrochloride salt (white solid, 140 mg, 0.253 mmol, 74%). LCMS: (ESI): m/z calcd. for C₂₃H₂₅ClFN₆O₂ 471.16 [M+H]⁺, found 471.2. ¹H NMR (400 MHz, CD₃OD) δ: 8.68 (s, 1H), 8.25 (s, 1H), 7.59 (d, J=3.7 Hz, 1H), 7.43 (s, 1H), 7.28 (d, J=10.9 Hz, 1H), 6.92 (d, J=3.5 Hz, 1H), 5.16-5.07 (m, 1H), 4.55 (dd, J=6.4, 7.5 Hz, 1H), 3.95 (d, J=6.4 Hz, 1H), 3.02-2.80 (m, 2H), 2.13-1.97 (m, 2H), 1.96-1.82 (m, 2H), 1.24 (s, 3H).

### Example 27

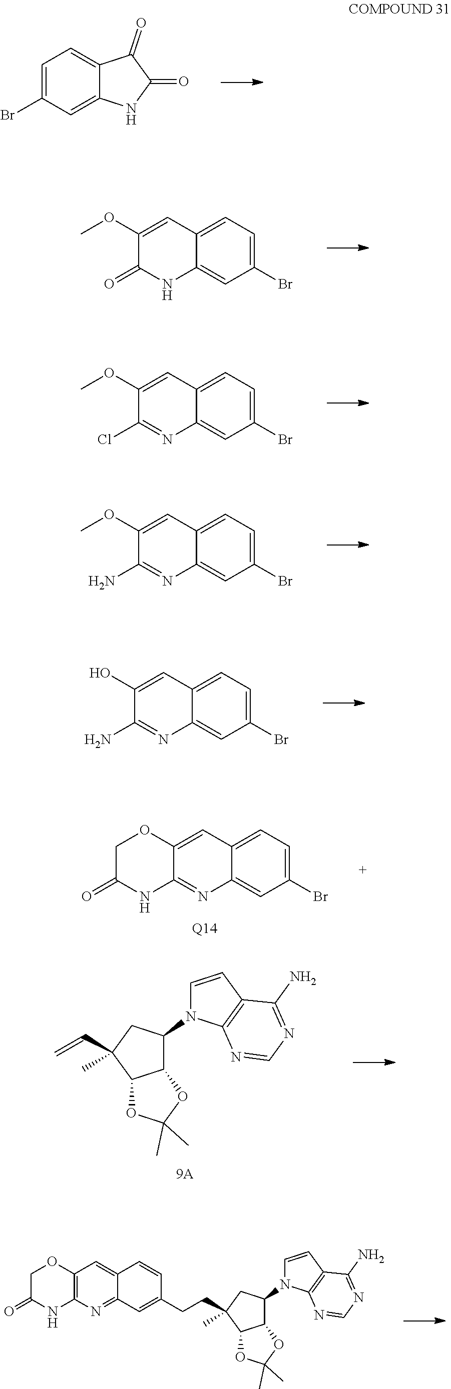

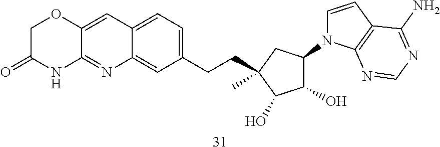 [SMILES: C[C@]1(CCc2ccc3cc4c(nc3c2)NC(=O)CO4)C[C@@H](n2ccc3c(N)ncnc32)[C@H](O)[C@@H]1O ; "31"]

To a solution of 6-bromoindoline-2,3-dione (5 g, 22.1 mmol) in EtOH (30 mL) was added diazomethyl(trimethyl)silane (5.1 g, 44.2 mmol) then followed by TEA (4.5 g, 44.2 mmol, 6.2 mL). The mixture was stirred at 20° C. for 18 h. The mixture was filtered, and the filter cake was washed with EtOH (30 mL) to afford 7-bromo-3-methoxy-1H-quinolin-2-one (3.1 g, 11.5 mmol, 52% yield, 94% purity) as a white solid. LCMS: (ESI): m/z calcd. for C₁₀H₉BrNO₂ 253.97 [M+H]⁺, found 253.9.

To a solution of 7-bromo-3-methoxy-1H-quinolin-2-one (1 g, 3.9 mmol) in toluene (20 mL) were added SOCl₂ (37.5 g, 314.9 mmol, 22.8 mL) and DMF (28.8 mg, 0.394 mmol, 0.030 mL) at 20° C. under N₂. The mixture was stirred at 80° C. for 2 h. The mixture was concentrated under reduced pressure to afford 7-bromo-2-chloro-3-methoxy-quinoline (1.05 g, 3.8 mmol, 96%) as a yellow solid, which was used for next step without further purification. LCMS: (ESI): m/z calcd. for C₁₀H₈BrClNO 273.94 [M+H]⁺, found 273.8.

A solution of 7-bromo-2-chloro-3-methoxy-quinoline (1.05 g, 3.8 mmol) in dioxane (20 mL) and NH₃.H₂O (20 mL, 25% wt) was stirred at 110° C. for 18 h in a 100 mL of sealed tube. The mixture was partitioned between brine (30 mL) and EA (30 mL). The organic phase was separated, washed with EA mL (3×30 mL), dried over Na₂SO₄, filtered and concentrated under reduced pressure. The residue was purified by silica gel chromatography (EA/PE gradient). 7-bromo-3-methoxy-quinolin-2-amine (590 mg, 2.1 mmol, 54% yield, 89% purity) was obtained as a yellow solid. LCMS: (ESI): m/z calcd. for C₁₀H₁₀BrN₂O 252.99 [M+H]⁺, found 253.0.

To a solution of 7-bromo-3-methoxy-quinolin-2-amine (590 mg, 2.1 mmol, 89% purity) in DCM (5 mL) was added BBr₃ (1.57 g, 6.3 mmol, 0.602 mL). The mixture was stirred at 20° C. for 2 h. The mixture was diluted with DCM (30 mL) and then quenched by addition of MeOH at 0° C. The mixture was concentrated under reduced pressure to give 2-amino-7-bromo-quinolin-3-ol hydrobromide (785 mg, crude) as a brown solid, which was used for the next step without further purification. LCMS: (ESI): m/z calcd. for C₉H₈BrN₂O 238.97 [M+H]⁺, found 240.9.

To a solution of 2-amino-7-bromo-quinolin-3-ol hydrobromide (685 mg, 2.1 mmol) in THF (34 mL) were added TEA (630.2 mg, 6.2 mmol, 0.867 mL) and 2-chloroacetyl chloride (304.8 mg, 2.7 mmol, 0.215 mL) at 0° C. The mixture was stirred at 20° C. for 2 h, then K₂CO₃ (573.8 mg, 4.2 mmol) was added. The mixture was stirred at 50° C. for 1 h. The reaction was quenched by addition of H₂O (20 mL) at 20° C., and then extracted with EA (3×40 mL). The combined organic layers were washed with NaCl (aq., 30 mL), dried over Na₂SO₄, filtered and concentrated under reduced pressure. The crude product was triturated with EA (50 mL) at 20° C. for 30 min. The solid was filtered, and the filter cake was dried under reduced pressure to afford 7-bromo-2H-[1,4]oxazino[3,2-b]quinolin-3(4H)-one (Q14) (426 mg, 1.5 mmol, 72%) as a yellow solid. LCMS: (ESI): m/z calcd. for C₁₁H₈BrN₂O₂ 278.97 [M+H]⁺, found 280.9.

A mixture of 7-[(3aR,4R,6R,6aS)-2,2,4-trimethyl-4-vinyl-3a,5,6,6a-tetrahydrocyclopenta[d][1,3]dioxol-6-yl]pyrrolo[2,3-d]pyrimidin-4-amine (9A) (150 mg, 472 μmol) and 9-BBN dimer (285.8 mg, 1.2 mmol) in THF (5 mL) was stirred at 50° C. for 1.5 h under Ar and then cooled to 20° C. K₃PO₄ (501.3 mg, 2.4 mmol) in water (1 mL) was added. The mixture was stirred at 20° C. for 0.5 h. Compound Q14 (173.3 mg, 614 μmol) and Pd(dppf)Cl₂ (34.6 mg, 0.047 mmol) were added. The mixture was stirred at 70° C. for 12 h under Ar. The mixture was partitioned between EA (20 mL) and water (20 mL). The organic phase was separated, and the aqueous phase was extracted with EA (3×30 mL). The organic phases were combined and washed with brine (30 mL), dried over anhydrous Na₂SO₄, filtered and concentrated under reduced pressure. The residue was purified by silica column chromatography (DCM/MeOH=100/1 to 100/3) to afford 7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-2H-[1,4]oxazino[3,2-b]quinolin-3 (4H)-one (144 mg, 277 μmol, 58%) as a yellow solid. LCMS: (ESI): m/z calcd. for C₂₈H₃₁N₆O₄ 515.23 [M+H]⁺, found 515.2.

To a solution of 7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-2H-[1,4]oxazino[3,2-h]quinolin-3(4H)-one (144 mg, 0.277 mmol) in THF (6 mL) was added HCl (4 M, 3 mL). The mixture was stirred at 20° C. for 12 h. The mixture was concentrated under reduced pressure. The residue was triturated with water/MeCN (10/1) at 20° C. for 1 h and then filtered to afford 7-(2-((1S,2R,3S,4R)-4-(4-amino-7H-pyrrolo[2,3-d]pyrimidin-7-yl)-2,3-dihydroxy-1-methylcyclopentyl)ethyl)-2H-[1,4]oxazino[3,2-h]quinolin-3(4H)-one as a hydrochloride salt (31) (white solid, 117 mg, 0.209 mmol, 76%). LCMS: (ESI): m/z calcd. for C₂₅H₂₇N₆O₄ 475.20 [M+H]⁺, found 475.2. ¹H NMR (400 MHz, CD₃OD) δ: 8.25 (s, 1H), 7.66-7.76 (m, 3H), 7.55 (d, J=3.4 Hz, 1H), 7.39 (br d, J=8.1 Hz, 1H), 6.91 (d, J=3.7 Hz, 1H), 5.11 (q, J=8.8 Hz, 1H), 4.76 (s, 2H), 4.58 (t, J=7.0 Hz, 1H), 3.95 (d, J=5.9 Hz, 1H), 2.75-2.97 (m, 2H), 1.99-2.13 (m, 2H), 1.91 (dt, J=11.2, 5.5 Hz, 2H), 1.25 (s, 3H).

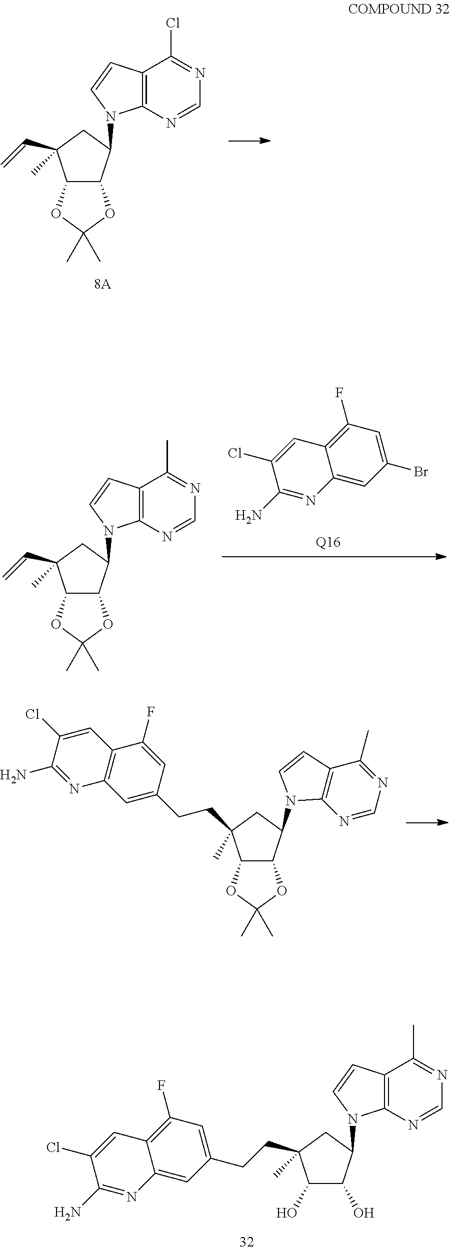

### Example 28

To a solution of 4-chloro-7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidine (300 mg, 0.899 mmol) in dioxane (5 mL) and H₂O (1 mL) were added 2,4,6-trimethyl-1,3,5,2,4,6-trioxatriborinane (676.92 mg, 2.70 mmol, 0.754 mL, 50% wt), Pd(dppf)Cl₂ (65.76 mg, 0.090 mmol), and K₃PO4 (953.83 mg, 4.49 mmol). The mixture was stirred at 90° C. for 12 h. The residue was diluted with water (5 mL) and extracted with EA (2×10 mL). The combined organic layers were washed with brine (10 mL), dried over Na₂SO₄. The solids were removed by filtration, and the filtrate was concentrated under reduced pressure. The residue was purified by silica gel chromatography (0˜25% EA/PE gradient) to afford 4-methyl-7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidine (189 mg, 597.05 μmol, 66%) as colorless oil. LCMS: (ESI): m/z calcd. for C₁₈H₂₄N₃O₂ 314.18 [M+H]⁺, found 314.1.

A mixture of 4-methyl-7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidine (189 mg, 0.597 mmol) and 9-BBN dimer (361.24 mg, 1.49 mmol) in THF (5 mL) was stirred at 50° C. for 2 h under N₂. The mixture was cooled to rt, and then a solution of K₃PO₄ (633.66 mg, 2.99 mmol) in H₂O (0.5 mL) was added. After stirring at rt for 0.5 h, 7-bromo-3-chloro-5-fluoro-quinolin-2-amine (246.73 mg, 0.896 mmol) and Pd(dppf)Cl₂ (43.69 mg, 0.060 mmol) were added. The mixture was stirred at 70° C. under N₂ for 12 h. The mixture was diluted with water (10 mL) and then extracted with EA (2×10 mL). The combined organic layers were washed with brine (10 mL), dried over Na₂SO₄, filtered and concentrated under reduced pressure. The residue was purified by silica column chromatography (PE:EA=3:1 to 1:1, then DCM:MeOH=100:1 to 20:1) followed by prep-HPLC purification (column: Phenomenex Gemini-NX 150×30 mm×5 μm; mobile phase: [water (0.04% NH₃H₂O+10 mM NH4HCO3)-ACN]; B %: 53%-83%, 8 min) to afford 3-chloro-5-fluoro-7-(2-((3aR,4S,6R,6aS)-2,2,4-trimethyl-6-(4-methyl-7H-pyrrolo[2,3-d]pyrimidin-7-yl)tetrahydro-4H-cyclopenta[d][1,3]dioxol-4-yl)ethyl)quinolin-2-amine (152 mg, 291.78 μmol, 49%) as an off-white solid. LCMS: (ESI): m/z calcd. for C₂₇H₃₀ClFN₅O₂ 510.20 [M+H]⁺, found 510.3.

To a solution of 3-chloro-5-fluoro-7-(2-((3aR,4S,6R,6aS)-2,2,4-trimethyl-6-(4-methyl-7H-pyrrolo[2,3-d]pyrimidin-7-yl)tetrahydro-4H-cyclopenta[d][1,3]dioxol-4-yl)ethyl)quinolin-2-amine (152 mg, 291.78 μmol) in THF (4 mL) was added HCl (4 M, 2 mL). The mixture was stirred at 25° C. for 7 h. The mixture was concentrated under reduced pressure. The residue was washed with MeCN:H₂O (10:1, 10 mL) to afford (1S,2R,3S,5R)-3-(2-(2-amino-3-chloro-5-fluoroquinolin-7-yl)ethyl)-3-methyl-5-(4-methyl-7H-pyrrolo[2,3-d]pyrimidin-7-yl)cyclopentane-1,2-diol (32) as a hydrochloride salt (white solid, 113 mg, 206.93 μmol, 71%). LCMS: (ESI): m/z calcd. for C₂₄H₂₆ClFN₅O₂ 470.17 [M+H]⁺, found 470.3. ¹H NMR (400 MHz, CD₃OD) δ: 8.97 (s, 1H), 8.60 (s, 1H), 8.06 (d, J=4.0 Hz, 1H), 7.43 (s, 1H), 7.24 (d, J=10.3 Hz, 1H), 7.17 (d, J=3.8 Hz, 1H), 5.32-5.24 (m, 1H), 4.62 (dd, J=6.3, 7.8 Hz, 1H), 3.98 (d, J=6.3 Hz, 1H), 2.98 (s, 3H), 2.97-2.82 (m, 2H), 2.16-2.04 (m, 2H), 1.98-1.86 (m, 2H), 1.26 (s, 3H).

### Example 29

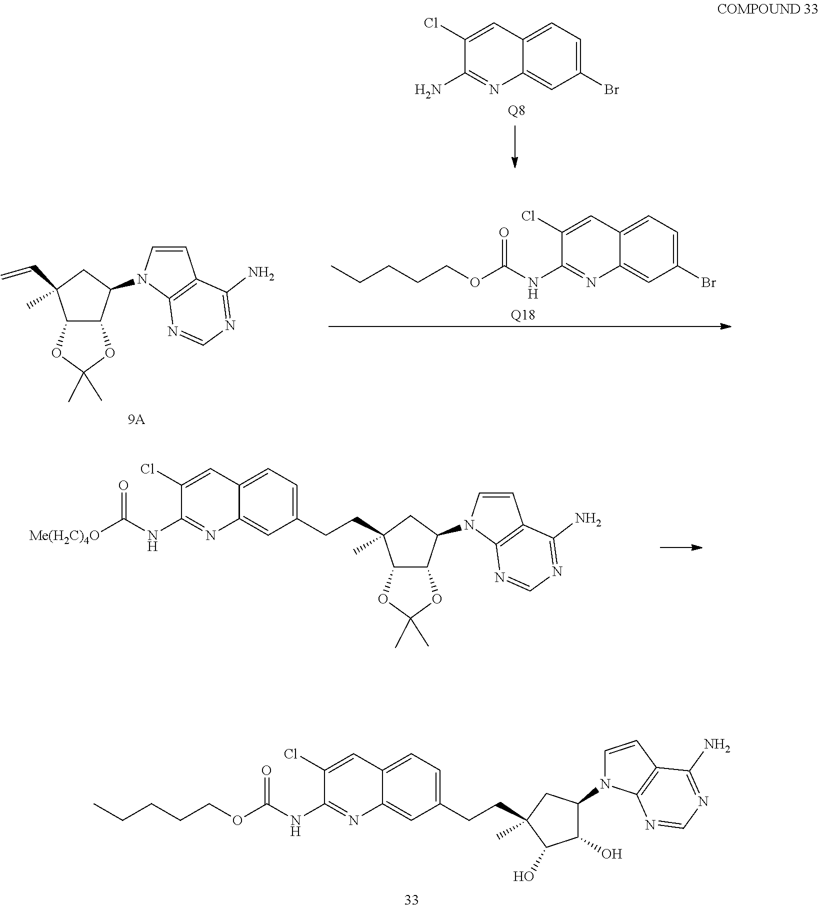

To a solution of 7-bromo-3-chloroquinolin-2-amine (Q8) (150 mg, 0.583 mmol) in DCM (3 mL) were added N-methylimidazole (286.95 mg, 3.49 mmol, 0.279 mL) and pentyl carbonochloridate (263.2 mg, 1.75 mmol) at 0° C. The mixture was stirred at rt for 12 h. The mixture was partitioned between EA (5 mL) and water (5 mL). The organic phase was separated, the aqueous phase extracted with EA (5 mL). The organic layers were combined, washed with brine (5 mL) and dried over Na₂SO₄, filtered and concentrated under reduced pressure. The residue was purified by silica gel chromatography (5%˜9% PE/EA gradient) to afford pentyl (7-bromo-3-chloroquinolin-2-yl)carbamate (Q18) (75 mg, 0.200 mmol, 34% yield, 99.1% purity) as a pale yellow solid. LCMS: (ESI): m/z calcd. for C₁₅H₁₇BrClN₂O₂ 373.01 [M+H]⁺, found 372.9.

A mixture of 7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidin-4-amine (9A) (170 mg, 540.74 mol) and 9-BBN dimer (327.18 mg, 1.35 mmol) in THF (5 mL) was stirred at 50° C. for 2 h under N₂ and then cooled to 20° C. A solution of K₃PO₄ (573.9 mg, 2.70 mmol) in water (0.5 mL) was added. The mixture was stirred at 20° C. for 0.5 h. Compound Q18 (241.2 mg, 0.649 mmol) and Pd(dppf)Cl₂ (39.57 mg, 54.07 μmol) were added. The mixture was stirred at 60° C. for 2 h under Ar. The mixture was partitioned between EA (5 mL) and water (5 mL). The organic phase was separated, and the aqueous phase washed with EA (5 mL). The organic layers were combined and washed with brine (5 mL), dried over Na₂SO₄, filtered and concentrated under reduced pressure. The residue was purified by column chromatography (SiO₂, PE:EA=2:1 then DCM:MeOH=100:1 to 30:1) to afford pentyl (7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-3-chloroquinolin-2-yl)carbamate (235 mg, 0.327 mmol, 60%, 84% purity) as a yellow solid. LCMS: (ESI): m/z calcd. for C₃₂H₄₀ClN₆O₄ 607.27 [M+H]⁺, found 607.5.

To a solution of pentyl (7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-3-chloroquinolin-2-yl)carbamate (185 mg, 257.17 μmol) in THF (4 mL) was added HCl (4 M, 2 mL). The mixture was stirred at rt for 12 h. The mixture was concentrated under reduced pressure. The residue was purified by prep-HPLC (Phenomenex Gemini-NX 80×40 mm×3 μm; mobile phase: [water (0.05% NH₃ in H₂O+10 mM NH₄HCO₃)-ACN]; B %: 39%-69%, 8 min) to afford pentyl (7-(2-((1S,2R,3S,4R)-4-(4-amino-7H-pyrrolo[2,3-d]pyrimidin-7-yl)-2,3-dihydroxy-1-methylcyclopentyl)ethyl)-3-chloroquinolin-2-yl)carbamate (33) (76 mg, 0.133 mmol, 52%) as a white solid. LCMS: (ESI): m/z calcd. for C₂₉H₃₆ClN₆O₄ 567.24 [M+H]⁺, found 567.2. ¹H NMR (400 MHz, CD₃OD) δ: 8.38 (s, 1H), 8.07 (s, 1H), 7.82 (s, 1H), 7.78 (d, J=8.3 Hz, 1H), 7.49 (d, J=7.0 Hz, 1H), 7.24 (d, J=3.8 Hz, 1H), 6.60 (d, J=3.5 Hz, 1H), 5.07-4.97 (m, 1H), 4.53 (t, J=6.9 Hz, 1H), 4.23 (t, J=6.7 Hz, 2H), 3.95 (d, J=6.3 Hz, 1H), 3.00-2.80 (m, 2H), 2.13-2.05 (m, 1H), 2.00-1.84 (m, 3H), 1.74 (quin, J=6.9 Hz, 2H), 1.48-1.34 (m, 4H), 1.26 (s, 3H), 0.98-0.91 (m, 3H).

### Example 30

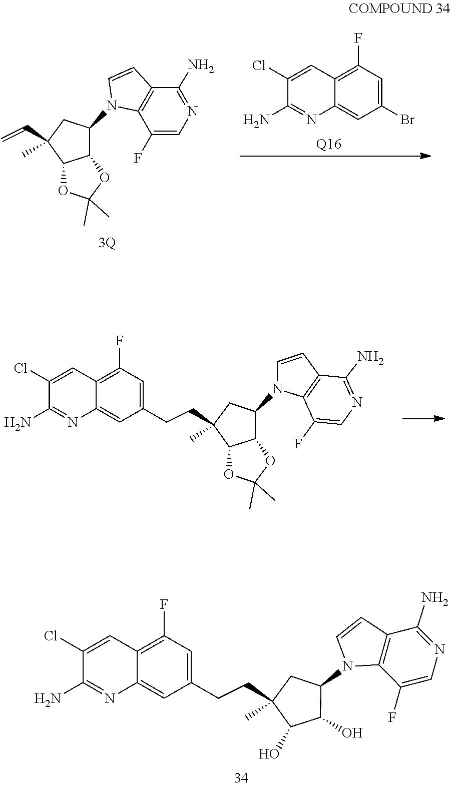

A mixture of 1-[(3aR,4R,6R,6aS)-2,2,4-trimethyl-4-vinyl-3a,5,6,6a-tetrahydrocyclopenta[d][1,3]dioxol-6-yl]-7-fluoro-pyrrolo[3,2-c]pyridin-4-amine (3Q) (140 mg, 0.422 mmol) and 9-BBN dimer (255.6 mg, 1.06 mmol) in THF (5 mL) was stirred at 50° C. for 1.5 h under Ar and then cooled to 20° C. A solution of K₃PO₄ (448.4 mg, 2.11 mmol) in H₂O (1 mL) was added. The mixture was stirred at 20° C. for 0.5 h. 7-bromo-3-chloro-5-fluoro-quinolin-2-amine (168.1 mg, 0.549 mmol) and Pd(dppf)Cl₂ (30.9 mg, 0.042 mmol) were added. The mixture was stirred at 70° C. for 12 h under Ar. The mixture was partitioned between EA (10 mL) and water (10 mL). The organic phase was separated, the aqueous phase washed with EA (3×10 mL). The organic layers were combined and washed with brine (10 mL), dried over Na₂SO₄, filtered and concentrated under reduced pressure. The residue was purified by silica column chromatography (PE:EA=1:1 then DCM:MeOH=20:1) to afford 7-[2-[(3aR,4S,6R,6aS)-6-(4-amino-7-fluoro-pyrrolo[3,2-c]pyridin-1-yl)-2,2,4-trimethyl-3a,5,6,6a-tetrahydrocyclopenta[d][1,3]dioxol-4-yl]ethyl]-3-chloro-5-fluoro-quinolin-2-amine (174 mg, 264.68 μmol, 63% yield, 80% purity) as a yellow solid. LCMS: (ESI): m/z calcd. for C₂₇H₂₉ClF₂N₅O₂ 528.19 [M+H]⁺, found 528.2.

To a solution of 7-[2-[(3aR,4S,6R,6aS)-6-(4-amino-7-fluoro-pyrrolo[3,2-c]pyridin-1-yl)-2,2,4-trimethyl-3a,5,6,6a-tetrahydrocyclopenta[d][1,3]dioxol-4-yl]ethyl]-3-chloro-5-fluoro-quinolin-2-amine (174 mg, 0.265 mmol, 80% purity) in THF (4 mL) was added HCl (4 M, 2 mL). The mixture was stirred at 20° C. for 2 h. The mixture was concentrated under reduced pressure to remove the solvent. The residue was purified by prep-HPLC (HCl conditions, column: Venusil ASB Phenyl 150×30 mm×5 μm; mobile phase: [water (0.05% HCl)-ACN]; B %: 17%-47%, 9 min) to afford (1S,2R,3S,5R)-3-[2-(2-amino-3-chloro-5-fluoro-7-quinolyl)ethyl]-5-(4-amino-7-fluoro-pyrrolo[3,2-c]pyridin-1-yl)-3-methyl-cyclopentane-1,2-diol (34) as a hydrochloride salt (off-white solid, 88 mg, 0.157 mmol, 59%). LCMS: (ESI): m/z calcd. for C₂₄H₂₅ClF₂N₅O₂ 488.16 [M+H]⁺, found 488.4. ¹H NMR (400 MHz, CD₃OD) δ: 8.68 (s, 1H), 7.74 (d, J=3.5 Hz, 1H), 7.60 (d, J=6.4 Hz, 1H), 7.44 (s, 1H), 7.29 (d, J=9.7 Hz, 1H), 7.13 (dd, J=3.1, 2.0 Hz, 1H), 5.09-5.20 (m, 1H), 4.40-4.46 (m, 1H), 3.92 (d, J=6.4 Hz, 1H), 2.82-2.99 (m, 2H), 2.16 (dd, J=12.9, 8.3 Hz, 1H), 1.79-1.95 (m, 3H), 1.23 (s, 3H).

### Example 31

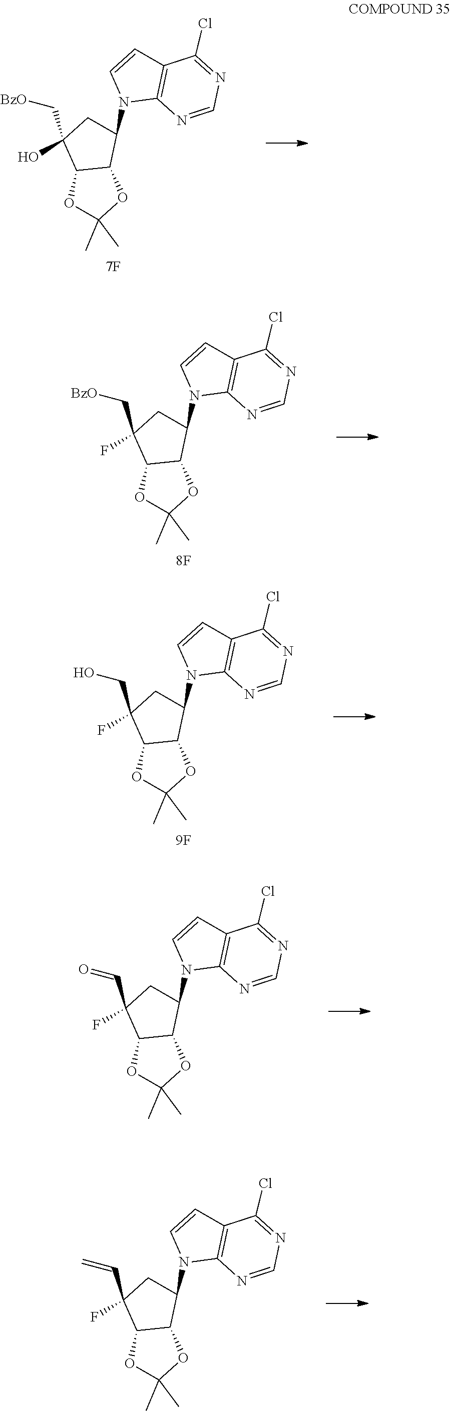

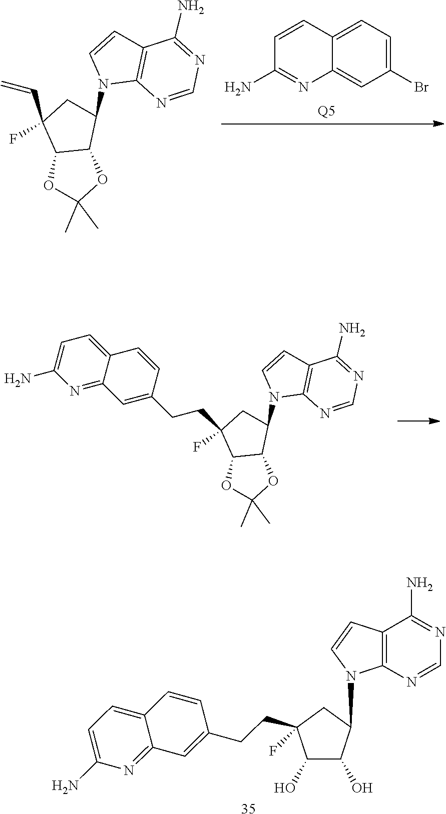

DAST (6.54 g, 40.55 mmol, 5.4 mL) was added dropwise to a solution of [(3aS,4R,6R,6aS)-6-(4-chloropyrrolo[2,3-d]pyrimidin-7-yl)-4-hydroxy-2,2-dimethyl-3a,5,6,6a-tetrahydrocyclopenta[d]J[1,3]dioxol-4-yl]methyl benzoate (4.5 g, 10.14 mmol) in DCM (50 mL) at 0° C. The mixture was stirred at 0° C. for 1 h, and then the reaction was quenched by NaHCO₃ (sat., aq., 100 mL). The mixture was extracted with DCM (2×50 mL). The separated organic layers were combined and washed with brine (20 mL) and dried over anhydrous Na₂SO₄. The solids were removed by filtration, and the filtrate was concentrated under reduced pressure. The residue was purified by silica gel column (PE/EA=8/1) to afford ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methyl benzoate (880 mg, 60% pure) as a white foam. LCMS: (ESI): m/z calcd. for C₂₂H₂₂ClFN₃O₄ 446.12 [M+H]⁺, found 446.2.

K₂CO₃ (53.5 mg, 386.8 μmol) was added to a solution of ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methyl benzoate (880 mg, 60% pure) in MeOH (15 mL) at 25° C. The mixture was stirred at 25° C. for 1 h, and then the reaction was quenched with AcOH (60 mg) and diluted with brine (10 mL). The mixture was extracted with EA (2×50 mL). The separated organic layers were combined and dried over anhydrous Na₂SO₄. The solids were removed by filtration, and the filtrate was concentrated under reduced pressure. The residue was purified by silica gel chromatography (PE:EA:EtOH=30:10:1) to afford ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methanol (570 mg, 60% pure) as a white foam. LCMS: (ESI): m/z calcd. for C₁₅H₁₈ClFN₃O₃ 342.09 [M+H]⁺, found 342.1.

To a solution of ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methanol (200 mg, 60% pure) in MeCN (5 mL) was added IBX (186.8 mg, 667.1 μmol). The mixture was stirred at 70° C. for 6 h. The mixture was cooled to rt and filtered. The filtrate was concentrated to afford (3aS,4R,6R,6aS)-6-(4-chloro-7H-pyrrolo[2,3-d]pyrimidin-7-yl)-4-fluoro-2,2-dimethyltetrahydro-4H-cyclopenta[d][1,3]dioxole-4-carbaldehyde (210 mg, 46% pure) as a white foam, which was used in the next step without further purification. LCMS: (ESI): m/z calcd. for C₁₅H₁₈ClFN₃O₄ 358.09 [M+H₃O]⁺, found 358.2.

A solution of tBuOK (123.1 mg, 1.10 mmol) in THF (2 mL) was added dropwise to a mixture of methyl(triphenyl)phosphonium bromide (420.0 mg, 1.18 mmol) in toluene (6 mL) at 25° C. The mixture was stirred at 25° C. for 1 h. (3aS,4R,6R,6aS)-6-(4-chloro-7H-pyrrolo[2,3-d]pyrimidin-7-yl)-4-fluoro-2,2-dimethyltetrahydro-4H-cyclopenta[d][1,3]dioxole-4-carbaldehyde (170 mg, 46% pure) in toluene (2 mL) was added dropwise at 0° C. After addition, the mixture was stirred at 25° C. for 16 h. NH₄Cl (aq., 5 mL) was added to quench the reaction. The mixture was extracted with EA (2×20 mL). The separated organic layers were combined, washed with brine (30 mL) and dried over anhydrous Na₂SO₄. The solids were removed by filtration, and the filtrate was concentrated under reduced pressure. The residue was purified by silica gel chromatography (PE:EA=100:0 to 85:15) to afford 4-chloro-7-((3aS,4R,6S,6aS)-6-fluoro-2,2-dimethyl-6-vinyltetrahydro-4H-cyclopenta[d][1,3]dioxol-4-yl)-7H-pyrrolo[2,3-d]pyrimidine (51 mg, 0.147 mmol) as a colorless gum. LCMS: (ESI): m/z calcd. for C₁₆H₁₈ClFN₃O₂ 338.10 [M+H]⁺, found 338.1.

A mixture of 4-chloro-7-((3aS,4R,6S,6aS)-6-fluoro-2,2-dimethyl-6-vinyltetrahydro-4H-cyclopenta[d][1,3]dioxol-4-yl)-7H-pyrrolo[2,3-d]pyrimidine (100 mg, 0.296 mmol) and NH₄OH (6.7 mL, aq., 25% wt) in dioxane (10 mL) was sealed in a tube and stirred at 100° C. for 36 h. The mixture was concentrated under reduced pressure, diluted with brine (10 mL) and then extracted with EA (2×20 mL). The separated organic layers were combined and dried over anhydrous Na₂SO₄. The solids were removed by filtration, and the filtrate was concentrated. The residue was purified by silica gel chromatography (PE:EA=3:1, 160 mL; then DCM:MeOH=20:1, 300 mL) to afford 7-((3aS,4R,6S,6aS)-6-fluoro-2,2-dimethyl-6-vinyltetrahydro-4H-cyclopenta[d][1,3]dioxol-4-yl)-7H-pyrrolo[2,3-d]pyrimidin-4-amine (68 mg, 0.207 mmol, 70%) as a white solid. LCMS: (ESI): m/z calcd. for C₁₆H₂₀FN₄O₂ 319.15 [M+H]⁺, found 319.3.

To a mixture of 7-((3aS,4R,6S,6aS)-6-fluoro-2,2-dimethyl-6-vinyltetrahydro-4H-cyclopenta[d][1,3]dioxol-4-yl)-7H-pyrrolo[2,3-d]pyrimidin-4-amine (62 mg, 195 μmol) in THF (3 mL) was added 9-BBN dimer (122.7 mg, 0.507 mmol). The mixture was stirred at 50° C. for 1 h and cooled to 20° C. A solution of K₃PO₄ (206.7 mg, 0.974 mmol) in H₂O (0.3 mL) was added. The mixture was stirred at 20° C. for 0.5 h, and then 7-bromoquinolin-2-amine (Q5) (60.8 mg, 273 μmol) and Pd(dppf)Cl₂ (14.2 mg, 0.019 mmol) were added. The mixture was stirred at 65° C. for 19 h. The mixture was diluted with brine (20 mL) and extracted with EA (6×30 mL). The separated organic layers were combined and dried over anhydrous Na₂SO₄. The solids were removed by filtration, and the filtrate was concentrated. The residue was purified by prep-HPLC (column: C18; mobile phase: [water (0.05% NH₃H₂O)-ACN]; B %: 5%-60%, 15 min) to afford 7-(2-((3aS,4R,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)ethyl)quinolin-2-amine (35 mg, 0.053 mmol, 27% yield, 70% purity) as white solid. LCMS: (ESI): m/z calcd. for C₂₅H₂₈FN₆O₂ 463.22 [M+H]⁺, found 463.4.

To a solution of 7-(2-((3aS,4R,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)ethyl)quinolin-2-amine (32.2 mg, 69.5% purity, 48.39 μmol) in THF (4.6 mL) was added HCl (4 M, 2.30 mL) at 25° C. The mixture was stirred at 25° C. for 16 h. The mixture was concentrated under reduced pressure. The residue was purified by pre-HPLC (column: Phenomenex Gemini-NX 150×30 mm×5 μm; mobile phase: [water (0.05% HCl)-ACN]; B %: 3%-30%, 7 min) to afford (1S,2S,3R,5R)-5-(4-amino-7H-pyrrolo[2,3-d]pyrimidin-7-yl)-3-(2-(2-aminoquinolin-7-yl)ethyl)-3-fluorocyclopentane-1,2-diol (35) as a hydrochloride salt (light yellow solid, 14 mg, 0.028 mmol, 58%). LCMS: (ESI): m/z calcd. for C₂₂H₂₄FN₆O₂ 423.19 [M+H]⁺, found 423.2. ¹H NMR (400 MHz, CD₃OD) δ: 8.31 (d, J=9.3 Hz, 1H), 8.26 (s, 1H), 7.83 (d, J=8.2 Hz, 1H), 7.58 (d, J=3.7 Hz, 1H), 7.55 (s, 1H), 7.47 (dd, J=1.4, 8.2 Hz, 1H), 7.01 (d, J=9.3 Hz, 1H), 6.93 (d, J=3.7 Hz, 1H), 5.20 (ddd, J=6.1, 8.6, 11.0 Hz, 1H), 4.47 (t, J=6.6 Hz, 1H), 4.29-4.17 (m, 1H), 3.17-2.95 (m, 2H), 2.67-2.52 (m, 1H), 2.46-2.10 (m, 3H). ¹⁹F NMR (376 MHz, CDCl₃) δ: 169.91.

### Example 32

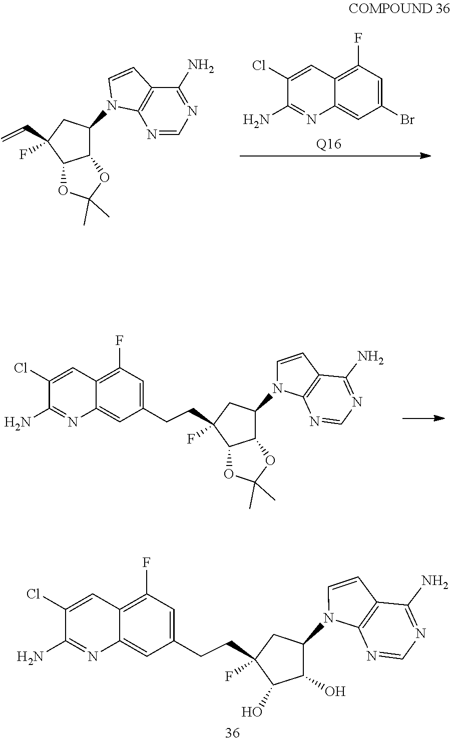

A mixture of 7-((3aS,4R,6S,6aS)-6-fluoro-2,2-dimethyl-6-vinyltetrahydro-4H-cyclopenta[d][1,3]dioxol-4-yl)-7H-pyrrolo[2,3-d]pyrimidin-4-amine (100 mg, 0.307 mmol) and 9-BBN dimer (185.8 mg, 767.88 μmol) in THF (5 mL) was stirred at 50° C. for 1.5 h under Ar and then cooled to 20° C. A solution of K₃PO₄ (326.0 mg, 1.54 mmol) in H₂O (1 mL) was added. The mixture was stirred at 20° C. for 0.5 h. 7-bromo-3-chloro-5-fluoro-quinolin-2-amine (Q16) (101.6 mg, 0.369 mmol) and Pd(dppf)Cl₂ (22.5 mg, 30.72 μmol) were added. The mixture was stirred at 60° C. for 12 h under Ar. The mixture was partitioned between EA (10 mL) and water (10 mL). The organic phase was separated, and the aqueous phase washed with EA (3×10 mL). The organic layers were combined, washed with brine (10 mL), dried over Na₂SO₄, filtered and concentrated under reduced pressure. The residue was purified by silica column chromatography (PE:EA=1:1 then DCM:MeOH=20:1) to afford 7-(2-((3aS,4R,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)ethyl)-3-chloro-5-fluoroquinolin-2-amine (134 mg, 0.234 mmol, 76%, 90% purity) as yellow gum. LCMS: (ESI): m/z calcd. for C₂₅H₂₆ClF₂N₆O₂ 515.17 [M+H]⁺, found 515.3.

To a solution of 7-(2-((3aS,4R,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)ethyl)-3-chloro-5-fluoroquinolin-2-amine (134 mg, 90% purity, 0.235 mmol) in THF (4 mL) was added HCl (4 M, 2 mL). The mixture was stirred at 25° C. for 12 h. The mixture was concentrated under reduced pressure. The residue was purified by prep-HPLC (Venusil ASB Phenyl 150×30 mm×5 μm; mobile phase: [water (0.05% HCl)-ACN]; B %: 10%-40%, 9 min) to afford (1S,2S,3R,5R)-3-(2-(2-amino-3-chloro-5-fluoroquinolin-7-yl)ethyl)-5-(4-amino-7H-pyrrolo[2,3-d]pyrimidin-7-yl)-3-fluorocyclopentane-1,2-diol (36) as a hydrochloride salt (off-white solid, 75 mg, 0.135 mmol, 62%). LCMS: (ESI): m/z calcd. for C₂₂H₂₂ClF₂N₆O₂ 475.14 [M+H]⁺, found 475.1. ¹H NMR (400 MHz, CD₃OD) δ: 8.69 (s, 1H), 8.27 (s, 1H), 7.59 (d, J=3.5 Hz, 1H), 7.46 (s, 1H), 7.31 (d, J=9.7 Hz, 1H), 6.93 (d, J=3.7 Hz, 1H), 5.19 (ddd, J=10.9, 8.4, 6.1 Hz, 1H), 4.47 (t, J=6.5 Hz, 1H), 4.18-4.29 (m, 1H), 2.97-3.16 (m, 2H), 2.59 (td, J=14.9, 8.6 Hz, 1H), 2.09-2.46 (m, 3H). ¹⁹F NMR (376 MHz, CD₃OD) δ: −121.36 (br d, J=10.3 Hz, 1F), −172.93-−167.03 (m, 1F).

### Example 33

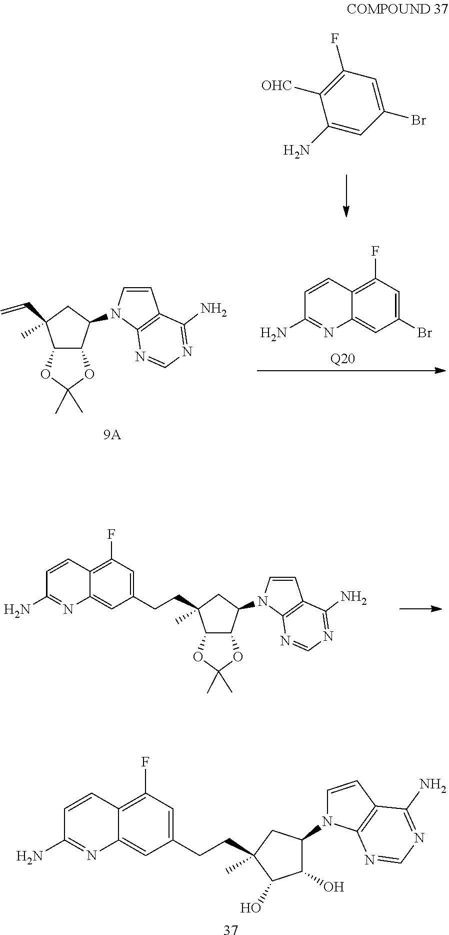

To a solution of 2-amino-4-bromo-6-fluoro-benzaldehyde (1 g, 4.59 mmol) and acetonitrile (376.58 mg, 9.17 mmol, 0.483 mL) in DMSO (20 mL) was added t-BuOK (1.03 g, 9.17 mmol) at 0° C. The mixture was stirred at rt for 15 min. The mixture was partitioned between EA (30 mL) and water (30 mL). The organic phase was separated, and the aqueous phase extracted with EA (30 mL). The organic layers were combined, washed with brine (50 mL) and dried over Na₂SO₄. The solids were removed by filtration, and the filtrate was concentrated under reduced pressure. The residue was purified by silica gel chromatography (0%˜35% PE/EA gradient) to afford 7-bromo-5-fluoroquinolin-2-amine (Q20) (715 mg, 2.97 mmol, 64%) as a yellow solid. LCMS: (ESI): m/z calcd. for C₉H₇BrFN₂ 242.97 [M+H]⁺, found 242.8.

A mixture of 7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidin-4-amine (9A) (100 mg, 0.318 mmol) and 9-BBN dimer (192.45 mg, 0.795 mmol) in THF (3 mL) was stirred at 50° C. for 2 h under N₂ and then cooled to 20° C. A solution of K₃PO₄ (337.59 mg, 1.59 mmol) in water (0.4 mL) was added. The mixture was stirred at 20° C. for 0.5 h. Compound Q20 (92.01 mg, 0.382 mmol) and Pd(dppf)Cl₂ (23.27 mg, 0.032 mmol) were added. The mixture was stirred at 60° C. for 12 h under Ar. The mixture was partitioned between EA (10 mL) and water (5 mL). The organic phase was separated, and the aqueous phase washed with EA (10 mL). The organic layers were combined, washed with brine (5 mL), dried over Na₂SO₄, filtered and concentrated under reduced pressure. The residue was purified by silica column chromatography (PE:EA=2:1 to 1:1 then DCM:MeOH=50:1 to 10:1) to afford 7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-5-fluoroquinolin-2-amine (149 mg, 0.300 mmol, 94% yield, 96% purity) as a white solid. LCMS: (ESI): m/z calcd. for C₂₆H₃₀FN₆O₂ 477.23 [M+H]⁺, found 477.2.

To a solution of 7-(2-((3aR,4S,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-5-fluoroquinolin-2-amine (149 mg, 0.300 mmol) in THF (4 mL) was added HCl (4 M, 2 mL). The mixture was stirred at rt for 12 h. The mixture was concentrated under reduced pressure. The residue was triturated with CH₃CN:H₂O (10:1, 20 mL) at rt for 30 min. The crude product was purified by prep-HPLC (column: Phenomenex Gemini-NX 80×40 mm×3 μm; mobile phase: [water (0.05% NH₃ in H₂O+10 mM NH₄HCO₃)-ACN]; B %: 20%-50%, 8 min) to afford (1S,2R,3S,5R)-3-(2-(2-amino-5-fluoroquinolin-7-yl)ethyl)-5-(4-amino-7H-pyrrolo[2,3-d]pyrimidin-7-yl)-3-methylcyclopentane-1,2-diol (37) (73 mg, 0.167 mmol, 56%) as a white solid. LCMS: (ESI): m/z calcd. for C₂₃H₂₆FN₆O₂ 437.20 [M+H]⁺, found 437.2. ¹H NMR (400 MHz, CD₃OD-d₄) δ: 8.08 (s, 1H), 8.06 (d, J=9.7 Hz, 1H), 7.24 (s, 1H), 7.19 (s, 1H), 6.87 (d, J=11.0 Hz, 1H), 6.81 (d, J=9.1 Hz, 1H), 6.60 (d, J=3.6 Hz, 1H), 5.05-4.97 (m, 1H), 4.53 (t, J=6.9 Hz, 1H), 3.93 (d, J=6.4 Hz, 1H), 2.88-2.70 (m, 2H), 2.11-2.03 (m, 1H), 1.96 (d, J=10.7 Hz, 1H), 1.93-1.80 (m, 2H), 1.23 (s, 3H). ¹⁹F NMR (376 MHz, CD₃OD-d₄) δ: −126.40 (1F).

### Example 34

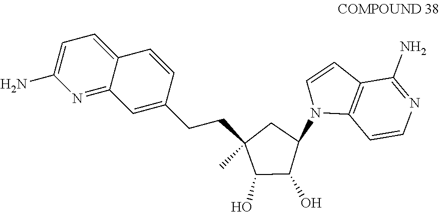 [SMILES: C[C@]1(CCc2ccc3ccc(N)nc3c2)C[C@@H](n2ccc3c(N)nccc32)[C@H](O)[C@@H]1O]

To a solution of 4-chloro-1H-pyrrolo[3,2-c]pyridine (554.30 mg, 3.63 mmol) in DMF (15 mL) was added t-BuOK (380.47 mg, 3.39 mmol). The mixture was stirred at 25° C. for 0.5 h followed by the addition of (3aR,4S,6R,6aR)-2,2,6-trimethyl-6-vinyltetrahydro-4H-cyclopenta[d][1,3]dioxol-4-yl trifluoromethanesulfonate (0.8 g, 2.42 mmol). The mixture was stirred at 25° C. for 12 h. The reaction progress was monitored by TLC (PE:EA=10:1). Upon completion, the mixture was diluted with water (30 mL) and then extracted with EA (2×30 mL). The separated organic layers were combined, washed with brine (60 mL), dried over anhydrous Na₂SO₄, and concentrated to afford a residue. The residue was purified by flash silica gel chromatography (eluent of 0˜4% Methanol/DCM gradient) to afford 4-chloro-1-((3aS,4R,6R,6aR)-2,2,6-trimethyl-6-vinyltetrahydro-4H-cyclopenta[d][1,3]dioxol-4-yl)-1H-pyrrolo[3,2-c]pyridine (0.29 g, 0.871 mmol, 36%) as white solid. LCMS: (ESI): RT=3.328 min, m/z calcd. for C₁₈H₂₂N₂O₂Cl 333.13, [M+H]⁺, found 333.1.

To a solution of 4-chloro-1-((3aS,4R,6R,6aR)-2,2,6-trimethyl-6-vinyltetrahydro-4H-cyclopenta[d][1,3]dioxol-4-yl)-1H-pyrrolo[3,2-c]pyridine (0.29 g, 0.871 mmol) and diphenylmethanimine (236.87 mg, 1.31 mmol, 0.219 mL) in toluene (8 mL) were added BINAP (108.51 mg, 0.174 mmol), Pd₂(dba)₃ (79.79 mg, 0.087 mmol) and t-BuONa (167.48 mg, 1.74 mmol). The mixture was stirred at 110° C. for 18 h under N₂. The reaction was quenched with NH₄Cl (sat., aq., 10 mL). The mixture was extracted with EA (3×10 mL). The separated organic layers were combined, washed with brine (30 mL), dried over anhydrous Na₂SO₄, filtered and concentrated to afford a crude imine intermediate. The crude intermediate was dissolved in MeOH (10 mL). Hydroxylamine (276.87 mg, 4.19 mmol, 50% wt in water) was added at 20° C. The mixture was stirred at 20° C. for 1 h. Upon completion of the reaction, the mixture was concentrated to dryness. The residue was purified by flash silica gel chromatography (eluent of 0˜5% Methanol/DCM gradient) to afford 1-((3aS,4R,6R,6aR)-2,2,6-trimethyl-6-vinyltetrahydro-4H-cyclopenta[d][1,3]dioxol-4-yl)-1H-pyrrolo[3,2-c]pyridin-4-amine (0.19 g, 69% yield over 2 steps) as a yellow semi-solid. LCMS: (ESI): RT=2.062 min, m/z calcd. for C₁₈H₂₄O₂N₃ 314.18, [M+H]⁺, found 314.1.

To a solution of 1-[(3aR,4R,6R,6aS)-2,2,4-trimethyl-4-vinyl-3a,5,6,6a-tetrahydrocyclopenta[d][1,3]dioxol-6-yl]pyrrolo[3,2-c]pyridin-4-amine (100 mg, 0.319 mmol) in THF (5 mL) was added 9-BBN dimer (115.84 mg, 0.479 mmol). The mixture was stirred at 50° C. for 1 h and then cooled to 30° C. A solution of K₃PO₄ (338.66 mg, 1.60 mmol) in H₂O (0.5 mL) was added. The mixture was stirred at 30° C. for 0.5 h, followed by addition of 7-bromoquinolin-2-amine (Q5) (85.41 mg, 0.383 mmol) and Pd(dppf)Cl₂ (23.35 mg, 0.032 mmol). The mixture was degassed (3×) and stirred at 60° C. for 10.5 h. Upon completion, the reaction was quenched with brine (20 mL), and then extracted with EA (3×20 mL). The combined organic layers were washed with brine (60 mL) and dried over anhydrous Na₂SO₄. The solids were removed by filtration, and the filtrate was concentrated under reduced pressure. The residue was purified by prep-HPLC (column: Phenomenex Gemini-NX 80×40 mm×3 um; mobile phase: [water (0.05% NH₃H₂O+10 mM NH₄HCO₃)-ACN]; B %: 17%-47%, 8 min) to afford 7-(2-((3aR,4S,6R,6aS)-6-(4-amino-1H-pyrrolo[3,2-c]pyridin-1-yl)-2,2,4-trimethyltetrahydro-4H-cyclopenta[d][1,3]dioxol-4-yl)ethyl)quinolin-2-amine (35 mg, 0.0744 mmol, 23%) as a brown semi-solid. LCMS: (ESI): RT=4.145 min, m/z calcd. for C₂₇H₃₂O₂N₅ 458.3, [M+H]⁺, found 458.4.

To a solution of 7-[2-[(3aR,4S,6R,6aS)-6-(4-aminopyrrolo[3,2-c]pyridin-1-yl)-2,2,4-trimethyl-3a, 5,6,6a-tetrahydrocyclopenta[d][1,3]dioxol-4-yl]ethyl]quinolin-2-amine (32 mg, 0.070 mmol) in THF (2 mL) was added HCl (4 M, 1 mL). The mixture was stirred at 20° C. for 4 h. Upon completion, the mixture was filtered and concentrated under reduced pressure to give a residue. The residue was purified by prep-HPLC (column: Phenomenex Gemini-NX 80×40 mm×3 um; mobile phase: [water (0.05% NH₃H₂O+10 mM NH₄HCO₃)-ACN]; B %: 19%-49%, 8 min) to afford (1S,2R,3S,5R)-5-(4-amino-1H-pyrrolo[3,2-c]pyridin-1-yl)-3-(2-(2-aminoquinolin-7-yl)ethyl)-3-methylcyclopentane-1,2-diol (38) (14 mg, 0.0326 mmol, 45%) as a white solid. LCMS: (ESI): RT=2.561 min, m/z calcd. for C₂₄H₂₈O₂N₅ 418.22, [M+H]⁺, found 418.3. ¹H NMR (400 MHz, CD₃OD) δ: 7.88 (d, J=8.8 Hz, 1H), 7.59-7.50 (m, 2H), 7.38 (s, 1H), 7.35 (d, J=3.5 Hz, 1H), 7.14 (dd, J=1.5, 8.2 Hz, 1H), 6.95 (d, J=6.6 Hz, 1H), 6.78-6.72 (m, 2H), 4.82-4.76 (m, 1H), 4.39-4.33 (m, 1H), 3.90 (d, J=6.2 Hz, 1H), 2.91-2.71 (m, 2H), 2.12 (dd, J=8.7, 13.1 Hz, 1H), 1.97-1.77 (m, 3H), 1.25 (s, 3H).

### Example 35

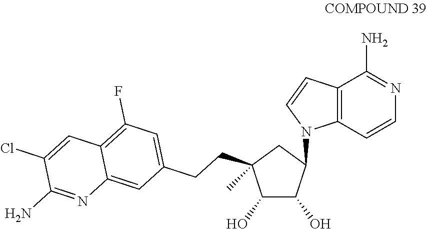 [SMILES: C[C@]1(CCc2cc(F)c3cc(Cl)c(N)nc3c2)C[C@@H](n2ccc3c(N)nccc32)[C@H](O)[C@@H]1O]

(1S,2R,3S,5R)-5-(4-amino-1H-pyrrolo[3,2-c]pyridin-1-yl)-3-(2-(2-amino-3-chloro-5-fluoroquinolin-7-yl)ethyl)-3-methylcyclopentane-1,2-diol was prepared similarly as described for (1S,2R,3S,5R)-5-(4-amino-1H-pyrrolo[3,2-c]pyridin-1-yl)-3-(2-(2-aminoquinolin-7-yl)ethyl)-3-methylcyclopentane-1,2-diol starting from (3aR,4S,6R,6aR)-2,2,6-trimethyl-6-vinyltetrahydro-4H-cyclopenta[d][1,3]dioxol-4-yl trifluoromethanesulfonate in a reaction with 7-bromo-3-chloro-5-fluoro-quinolin-2-amine (Q16). LCMS: (ESI): RT=2.905 min, m/z calcd. for C₂₄H₂₆O₂N₅ClF 470.17, [M+H]⁺, found 470.2. ¹H NMR (400 MHz, CD₃OD) δ=8.18 (s, 1H), 7.54 (d, J=6.2 Hz, 1H), 7.29 (d, J=3.3 Hz, 1H), 7.23 (s, 1H), 6.92 (dd, J=1.0, 10.9 Hz, 1H), 6.88 (d, J=6.3 Hz, 1H), 6.67 (d, J=3.2 Hz, 1H), 4.82-4.73 (m, 1H), 4.35 (t, J=6.9 Hz, 1H), 3.90 (d, J=6.3 Hz, 1H), 2.90-2.70 (m, 2H), 2.10 (dd, J=8.6, 13.1 Hz, 1H), 1.95-1.75 (m, 3H), 1.23 (s, 3H). ¹⁹F NMR (376 MHz, CD₃OD) δ: −125.76 (s, 1F).

### Example 36

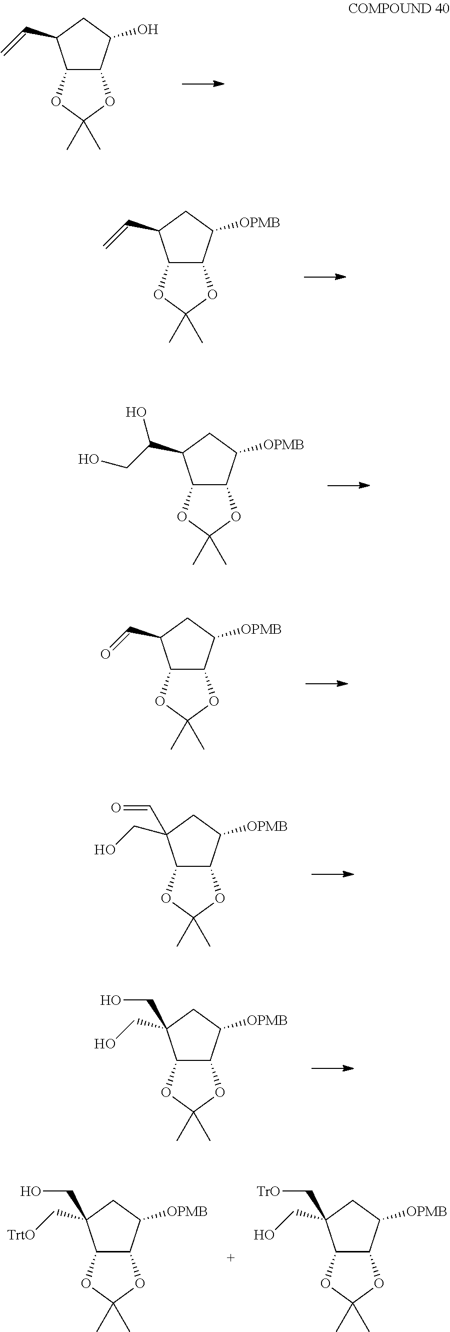

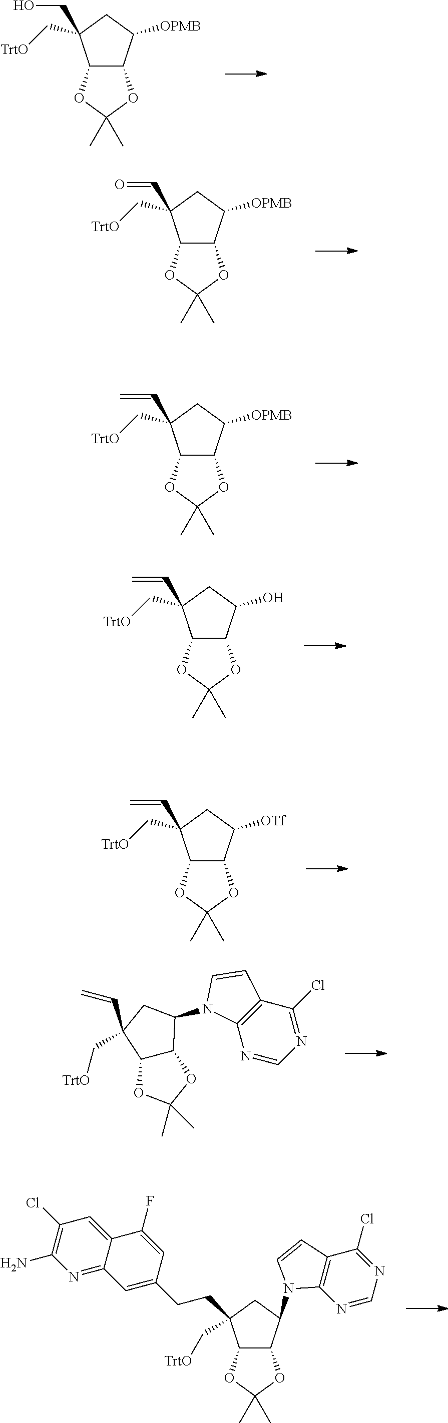

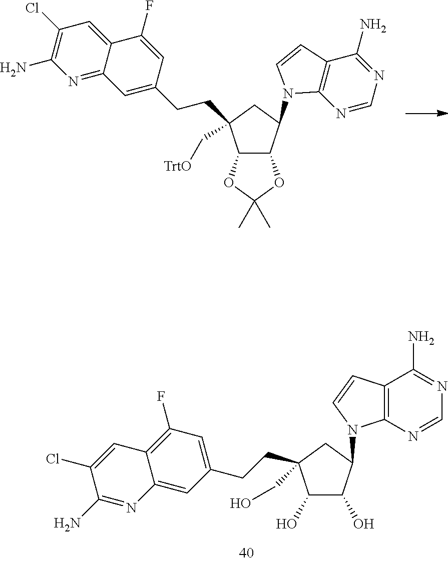

To a solution of (3aS,4S,6R,6aR)-2,2-dimethyl-6-vinyltetrahydro-3aH-cyclopenta[d][1,3]dioxol-4-ol (7.4 g, 40.17 mmol, 1 eq.) in DMF (40 mL) was added NaH (3.21 g, 80.33 mmol, 60% purity, 2.0 eq.) at 0° C. After stirring at 0° C. for 0.5 h, PMB-Cl (11.32 g, 72.30 mmol, 9.85 mL, 1.8 eq.) was added, and the mixture was stirred at 25° C. for 2 h. The reaction progress was monitored by TLC (PE:EtOAc=5:1). Upon completion, the reaction was quenched by addition of NH₄Cl (40 mL), and then extracted with EtOAc (3×100 mL). The combined organic layers was washed with brine 300 mL (3×100 mL), dried over anhydrous Na₂SO₄, filtered and concentrated under reduced pressure to give a residue. The residue was purified by flash silica gel chromatography (ISCO®; 120 g SepaFlash® Silica Flash Column, Eluent of 0˜10% EA/PE gradient @ 45 mL/min) to afford (3aS,4S,6R,6aR)-4-((4-methoxybenzyl)oxy)-2,2-dimethyl-6-vinyltetrahydro-3aH-cyclopenta[d][1,3]dioxole (7.6 g, 24.97 mmol) as a colorless oil.

To a solution of (3aS,4S,6R,6aR)-4-((4-methoxybenzyl)oxy)-2,2-dimethyl-6-vinyltetrahydro-3aH-cyclopenta[d][1,3]dioxole (22.26 g, 73.13 mmol, 1 eq.) in a mixed solvent of THF (150 mL) and H₂O (150 mL) were added K₂OsO₄ (2.09 g, 10.97 mmol, 0.15 eq.) and NMO (17.13 g, 146.26 mmol, 2.0 eq.). The mixture was stirred at 25° C. for 18 h. Upon completion, the reaction was quenched by the addition of sat. aq. Na₂S₂O₃ (100 mL), and then extracted with EtOAc (3×200 mL). The combined organic layers was washed with brine (300 mL), dried over anhydrous Na₂SO₄, filtered and concentrated under reduced pressure to give a residue. The residue was purified by column chromatography (SiO₂, DCM:MeOH=30:1 to 10:1) to afford 1-((3aR,4R,6S,6aS)-6-((4-methoxybenzyl)oxy)-2,2-dimethyltetrahydro-4H-cyclopenta[d][1,3]dioxol-4-yl)ethane-1,2-diol (24.3 g, 71.81 mmol, 98% yield) as a yellow oil. LCMS: (ESI): m/z calcd. for C₁₈H₂₆O₆Na, 361.17 [M+Na]⁺, found 361.1.

To a mixture of 1-((3aR,4R,6S,6aS)-6-((4-methoxybenzyl)oxy)-2,2-dimethyltetrahydro-4H-cyclopenta[d][1,3]dioxol-4-yl)ethane-1,2-diol (24.3 g, 71.81 mmol, 1 eq.) in THF (50 mL) and H₂O (50 mL) was added NaIO₄ (15.36 g, 71.81 mmol, 3.98 mL, 1.0 eq.). The mixture was stirred at 25° C. for 1 h. Upon completion, the mixture was diluted by the addition water (50 mL), and then extracted with EtOAc (3×100 mL). The combined organic layers were washed with brine (100 mL), dried over anhydrous Na₂SO₄, filtered and concentrated under reduced pressure to give a residue. The residue was concentrated under reduced pressure to give (17.53 g, 57.22 mmol, crude) as a colorless oil. LCMS: (ESI): m/z calcd. for C₁₇H₂₄O₆Na, 347.2 [M+H₂O+Na]⁺, found 347.0.

To a solution of (3aR,4S,6S,6aS)-6-((4-methoxybenzyl)oxy)-2,2-dimethyltetrahydro-3aH-cyclopenta[d][1,3]dioxole-4-carbaldehyde (17.53 g, 57.22 mmol, 1 eq.) in dioxane (20 mL) were added KOH (2 M, 57.22 mL, 2 eq.) and HCHO (37.15 g, 457.77 mmol, 34.08 mL, 37% aq. solution, 8 eq.). The mixture was stirred at 25° C. for 2 h. The reaction was quenched with H₂O (30 mL), and then extracted with EtOAc (3×100 mL). The combined organic layers was washed with brine (100 mL), dried over anhydrous Na₂SO₄, filtered and concentrated under reduced pressure to give crude (3aR,6S,6aS)-4-(hydroxymethyl)-6-((4-methoxybenzyl)oxy)-2,2-dimethyltetrahydro-4H-cyclopenta[d][1,3]dioxole-4-carbaldehyde.

To a solution of crude (3aR,6S,6aS)-4-(hydroxymethyl)-6-((4-methoxybenzyl)oxy)-2,2-dimethyltetrahydro-4H-cyclopenta[d][1,3]dioxole-4-carbaldehyde in MeOH (20 mL) was added NaBH₄ (6.49 g, 171.66 mmol, 3 eq.) at 0° C., and the mixture was stirred at 0° C. for 1 h. The reaction progress was monitored by TLC (PE:EtOAc=0:1). Upon completion, the reaction was quenched by sat. NH₄Cl solution (30 mL), and then extracted with EtOAc (3×100 mL). The combined organic layers was washed with brine (100 mL), dried over anhydrous Na₂SO₄, filtered and concentrated under reduced pressure to give a residue. The residue was purified by flash silica gel chromatography (ISCO®; 120 g SepaFlash® Silica Flash Column, Eluent of 0˜79% EA/PE ether gradient @ 45 mL/min) to afford ((3aR,6S,6aS)-6-((4-methoxybenzyl)oxy)-2,2-dimethyltetrahydro-3aH-cyclopenta[d][1,3]dioxole-4,4-diyl)dimethanol (16 g, 47.28 mmol, 83% yield) as a colorless oil. LCMS: (ESI): m/z calcd. for C₁₈H₂₆O₆Na, 361.17 [M+Na]⁺, found 361.0.

To a mixture of ((3aR,6S,6aS)-6-((4-methoxybenzyl)oxy)-2,2-dimethyltetrahydro-3aH-cyclopenta[d][1,3]dioxole-4,4-diyl)dimethanol (16 g, 47.28 mmol, 1 eq.) in DCM (100 mL) were added TEA (47.84 g, 472.82 mmol, 65.81 mL, 10 eq.) and TrtCl (15.82 g, 56.74 mmol, 1.2 eq.) at 0° C. The mixture was stirred at 25° C. for 12 h. The reaction progress was monitored by TLC (PE:EtOAc=0:1 and then toluene:EA=5:1). Upon completion, the reaction was quenched by the addition of HCl (4 M, 15 mL), and then extracted with EtOAc (3×200 mL). The combined organic layers were washed with brine (200 mL), dried over anhydrous Na₂SO₄, filtered and concentrated under reduced pressure to give a residue. The residue was purified by silica gel flash chromatography (ISCO®; 220 g SepaFlash® Silica Flash Column, Eluent of 0-20% EA/PE gradient @ 65 mL/min), followed by re-purification by silica gel flash chromatography (ISCO®; 120 g SepaFlash® Silica Flash Column, Eluent of 0-7% EA/toluene ether gradient @ 45 mL/min) to afford ((3aR,4R,6S,6aS)-6-((4-methoxybenzyl)oxy)-2,2-dimethyl-4-((trityloxy)methyl)tetrahydro-4H-cyclopenta[d][1,3]dioxol-4-yl)methanol (12.59 g, 21.68 mmol, 46% yield) and ((3aR,4S,6S,6aS)-6-((4-methoxybenzyl)oxy)-2,2-dimethyl-4-((trityloxy)methyl)tetrahydro-4H-cyclopenta[d][1,3]dioxol-4-yl)methanol (2.65 g, 4.56 mmol, 10% yield). ((3aR,4R,6S,6aS)-6-((4-methoxybenzyl)oxy)-2,2-dimethyl-4-((trityloxy)methyl)tetrahydro-4H-cyclopenta[d][1,3]dioxol-4-yl)methanol: LCMS: (ESI): m/z calcd. for C₃₇H₄₀O₆Na, 603.28 [M+Na]⁺, found 603.3.

To a mixture of ((3aR,4R,6S,6aS)-6-((4-methoxybenzyl)oxy)-2,2-dimethyl-4-((trityloxy)methyl)tetrahydro-4H-cyclopenta[d][1,3]dioxol-4-yl)methanol (6.89 g, 11.86 mmol, 1 eq.) in EtOAc (60 mL) was added IBX (4.98 g, 17.80 mmol, 1.5 eq.). The mixture was stirred at 60° C. for 12 h. The reaction progress was monitored by TLC (toluene:EA=3:1). Upon completion, the mixture was filtered and concentrated under reduced pressure to afford (3aR,4R,6S,6aS)-6-((4-methoxybenzyl)oxy)-2,2-dimethyl-4-((trityloxy)methyl)tetrahydro-3aH-cyclopenta[d][1,3]dioxole-4-carbaldehyde (7.18 g, crude) as a colorless oil. The crude was used for the next step without further purification.

To a mixture of methyl triphenyl phosphonium bromide (24.27 g, 67.95 mmol, 5.5 eq.) and THF (200 mL) was added t-BuOK (6.93 g, 61.78 mmol, 5 eq.) at 0° C. The mixture was stirred at 0° C. for 0.5 h and then (3aR,4R,6S,6aS)-6-((4-methoxybenzyl)oxy)-2,2-dimethyl-4-((trityloxy)methyl)tetrahydro-3aH-cyclopenta[d][1,3]dioxole-4-carbaldehyde (7.15 g, 12.36 mmol, 1 eq.) was added. The mixture was stirred at 25° C. for 1 h. The reaction progress was monitored by TLC (PE:EtOAc=5:1). Upon completion, the reaction was quenched by the addition of sat. NH₄Cl solution (20 mL), and then extracted with EtOAc (3×100 mL). The combined organic layers were washed with brine (3×100 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0-10% EA/PE gradient @ 45 mL/min) to afford (3aR,4R,6S,6aS)-6-((4-methoxybenzyl)oxy)-2,2-dimethyl-4-((trityloxy)methyl)-4-vinyltetrahydro-3aH-cyclopenta[d][1,3]dioxole (5.54 g, 9.61 mmol, 78% yield) as a colorless oil.

To a mixture of (3aR,4R,6S,6aS)-6-((4-methoxybenzyl)oxy)-2,2-dimethyl-4-((trityloxy)methyl)-4-vinyltetrahydro-3aH-cyclopenta[d][1,3]dioxole (5.54 g, 9.61 mmol, 1 eq.) in a mixed solvent of PBS buffer (pH=7.4, 10 mL) and DCM (10 mL) was added DDQ (6.54 g, 28.82 mmol, 3 eq.). The mixture was stirred at 25° C. for 2 h. The reaction progress was monitored by TLC (PE:EtOAc=5:1). Upon completion, the reaction was quenched by addition of water (20 mL), and then extracted with EtOAc (3×100 mL). The combined organic layers was washed with brine (3×100 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0-25% EA/PE gradient @ 45 mL/min) to afford (3aS,4S,6R,6aR)-2,2-dimethyl-6-((trityloxy)methyl)-6-vinyltetrahydro-3aH-cyclopenta[d][1,3]dioxol-4-ol (3.97 g, 7.65 mmol, 80% yield, 88% purity) as a colorless oil.

To a solution of (3aS,4S,6R,6aR)-2,2-dimethyl-6-((trityloxy)methyl)-6-vinyltetrahydro-3aH-cyclopenta[d][1,3]dioxol-4-ol (1.95 g, 4.34 mmol, 1 eq.) in DCM (20 mL) and pyridine (1.37 g, 17.35 mmol, 1.40 mL, 4 eq.) was added dropwise Tf₂O (1.84 g, 6.51 mmol, 1.07 mL, 1.5 eq.) at 0° C. The mixture was stirred at 0° C. for 1 h. The reaction progress was monitored by TLC (PE:EtOAc=5:1). Upon completion, the reaction was quenched by the addition of ice water (20 mL), and then extracted with DCM (3×50 mL). The combined organic layers were washed with brine (150 mL), dried over anhydrous Na₂SO₄, filtered and concentrated under reduced pressure to give a residue. The residue was concentrated under reduced pressure to afford (3aR,4S,6R,6aR)-2,2-dimethyl-6-((trityloxy)methyl)-6-vinyltetrahydro-3aH-cyclopenta[d][1,3]dioxol-4-yl trifluoromethanesulfonate (2.43 g, crude) as a colorless oil.

To a solution of (3aR,4S,6R,6aR)-2,2-dimethyl-6-((trityloxy)methyl)-6-vinyltetrahydro-3aH-cyclopenta[d][1,3]dioxol-4-yl trifluoromethanesulfonate (4.2 g, 7.14 mmol, 1 eq.) in DMF (50 mL) was added the potassium salt of 4-chloro-7H-pyrrolo[2,3-d]pyrimidine (7-a) (2.05 g, 10.70 mmol, 1.5 eq.). The mixture was stirred at 25° C. for 12 h. Upon completion, the mixture was diluted with H₂O (50 mL) and extracted with EtOAc (3×50 mL). The combined organic layers was washed with brine (150 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜10% EA/PE gradient @ 35 mL/min) to afford 4-chloro-7-((3aS,4R,6R,6aR)-2,2-dimethyl-6-((trityloxy)methyl)-6-vinyltetrahydro-3aH-cyclopenta[d][1,3]dioxol-4-yl)-7H-pyrrolo[2,3-d]pyrimidine (1.98 g, 3.01 mmol, 44% yield, 90% purity) as a white solid. LCMS: (ESI): m/z calcd. for C₃₆H₃₅ClN₃O₃, 592.23 [M+H]⁺, found 592.2.

To a solution of 4-chloro-7-((3aS,4R,6R,6aR)-2,2-dimethyl-6-((trityloxy)methyl)-6-vinyltetrahydro-3aH-cyclopenta[d][1,3]dioxol-4-yl)-7H-pyrrolo[2,3-d]pyrimidine (500 mg, 0.844 mmol, 1 eq.) in THF (10 mL) was added 9-BBN dimer (226.67 mg, 1.86 mmol, 2.2 eq.). The mixture was stirred at 50° C. for 2 h under Ar, and then cooled to 25° C. A solution of K₃PO₄ (896.20 mg, 4.22 mmol, 5 eq.) in H₂O (1 mL) was added, and the mixture was stirred for 0.5 h. 7-bromo-3-chloro-5-fluoro-quinolin-2-amine (Q16) (302.43 mg, 1.10 mmol, 1.3 eq.) and Pd(dppf)Cl₂ (61.79 mg, 84.44 μmol, 0.1 eq.) were added, and the mixture was stirred at 60° C. for 12 h. The reaction progress was monitored by TLC (DCM:MeOH=10:1). Upon completion, the reaction was quenched by addition of water (20 mL), and then extracted with EtOAc (3×30 mL). The combined organic layers were washed with brine (60 mL), dried over anhydrous Na₂SO₄, filtered and concentrated under reduced pressure to give a residue. The residue was purified by flash silica gel chromatography (ISCO®; 24 g SepaFlash® Silica Flash Column, Eluent of 0˜48% EA/PE gradient @ 35 mL/min) to afford 3-chloro-7-(2-((3aR,4S,6R,6aS)-6-(4-chloro-7H-pyrrolo[2,3-d]pyrimidin-7-yl)-2,2-dimethyl-4-((trityloxy)methyl)tetrahydro-3aH-cyclopenta[d][1,3]dioxol-4-yl)ethyl)-5-fluoroquinolin-2-amine (349 mg, 0.450 mmol, 50% yield) as a yellow solid. LCMS: (ESI): m/z calcd. for C₄₅H₄₁Cl₂FN₅O₃, 788.3 [M+H]⁺, found 788.3.

To a solution of 3-chloro-7-(2-((3aR,4S,6R,6aS)-6-(4-chloro-7H-pyrrolo[2,3-d]pyrimidin-7-yl)-2,2-dimethyl-4-((trityloxy)methyl)tetrahydro-3aH-cyclopenta[d][1,3]dioxol-4-yl)ethyl)-5-fluoroquinolin-2-amine (340 mg, 0.431 mmol, 1 eq.) in dioxane (4 mL) was added NH₃.H₂O (3.64 g, 29.08 mmol, 4 mL, 28% purity, 67.46 eq.). The mixture was stirred at 110° C. for 12 h in a 30 mL sealed tube. The reaction progress was monitored by TLC (DCM:MeOH=10:1). Upon completion, the residue was diluted with NH₄Cl (10 mL) and extracted with EtOAc (3×50 mL). The combined organic layers were washed with brine (50 mL), dried over anhydrous Na₂SO₄, filtered and concentrated under reduced pressure to give a residue. The residue was purified by flash silica gel chromatography (ISCO®; 24 g SepaFlash® Silica Flash Column, Eluent of 0˜4% MeOH/DCM @ 35 mL/min) to afford 7-(2-((3aR,4S,6R,6aS)-6-(4-amino-7H-pyrrolo[2,3-d]pyrimidin-7-yl)-2,2-dimethyl-4-((trityloxy)methyl)tetrahydro-3aH-cyclopenta[d][1,3]dioxol-4-yl)ethyl)-3-chloro-5-fluoroquinolin-2-amine (200 mg, 0.247 μmol, 57% yield, 95% purity) as a white solid. LCMS: (ESI): m/z calcd. for C₄₅H₄₃ClFN₆O₃, 769.3 [M+H]⁺, found 769.3.

A solution of 7-(2-((3aR,4S,6R,6aS)-6-(4-amino-7H-pyrrolo[2,3-d]pyrimidin-7-yl)-2,2-dimethyl-4-((trityloxy)methyl)tetrahydro-3aH-cyclopenta[d][1,3]dioxol-4-yl)ethyl)-3-chloro-5-fluoroquinolin-2-amine (200 mg, 259.98 μmol, 1 eq.) in HCl (4M, aq., 1 mL) and THF (2 mL) was stirred at 25° C. for 12 h. The reaction progress was monitored by TLC (DCM:MeOH=10:1). Upon completion, the mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 21%-45%, 8 min) to afford (1S,2R,3S,5R)-3-(2-(2-amino-3-chloro-5-fluoroquinolin-7-yl)ethyl)-5-(4-amino-7H-pyrrolo[2,3-d]pyrimidin-7-yl)-3-(hydroxymethyl)cyclopentane-1,2-diol (40) (75 mg, 154.03 μmol, 59% yield, 100% purity) as a white solid. LCMS: (ESI): m/z calcd. for C₂₃H₂₅ClFN₆O₃ 487.16. [M+H]⁺, found 487.3. ¹H NMR (400 MHz, CD₃OD) δ: 8.19 (s, 1H), 8.13-8.00 (m, 1H), 7.28 (s, 1H), 7.24 (d, J=3.5 Hz, 1H), 6.97 (br d, J=10.6 Hz, 1H), 6.59 (d, J=3.5 Hz, 1H), 5.06-4.99 (m, 1H), 4.67-4.62 (m, 1H), 4.03 (d, J=5.5 Hz, 1H), 3.86 (br d, J=11.2 Hz, 1H), 3.74 (br d, J=11.2 Hz, 1H), 2.87 (dt, J=5.4, 12.6 Hz, 1H), 2.81-2.66 (m, 1H), 2.17 (br dd, J=9.2, 13.3 Hz, 1H), 2.07-1.83 (m, 3H). ¹⁹F NMR (376 MHz, CD₃OD) δ: −125.85 (s, 1F).

### Example 37

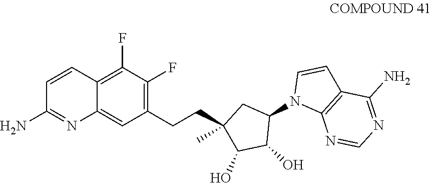 [SMILES: C[C@]1(CCc2cc3nc(N)ccc3c(F)c2F)C[C@@H](n2ccc3c(N)ncnc32)[C@H](O)[C@@H]1O]

(1S,2R,3S,5R)-3-(2-(2-amino-5,6-difluoroquinolin-7-yl)ethyl)-5-(4-amino-7H-pyrrolo[2,3-d]pyrimidin-7-yl)-3-methylcyclopentane-1,2-diol (41) was obtained as a hydrochloride salt and was prepared similarly as described for (1S,2R,3S,5R)-3-(2-(2-amino-5-fluoroquinolin-7-yl)ethyl)-5-(4-amino-7H-pyrrolo[2,3-d]pyrimidin-7-yl)-3-methylcyclopentane-1,2-diol starting from 7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidin-4-amine (9A) in a reaction with 7-bromo-5,6-difluoro-quinolin-2-amine (Q21). LCMS: (ESI): m/z calcd. for C₂₃H₂₅F₂N₆O₂ 455.19 [M+H]⁺, found 455.2. ¹H NMR (400 MHz, CD₃OD) δ: 8.45 (d, J=9.5 Hz, 1H), 8.25 (s, 1H), 7.58 (d, J=3.7 Hz, 1H), 7.43 (br d, J=4.4 Hz, 1H), 7.13 (d, J=9.5 Hz, 1H), 6.92 (d, J=3.7 Hz, 1H), 5.06-5.20 (m, 1H), 4.55 (t, J=6.9 Hz, 1H), 3.97 (d, J=6.4 Hz, 1H), 2.85-3.09 (m, 2H), 1.97-2.16 (m, 2H), 1.81-1.96 (m, 2H), 1.26 (s, 3H). ¹⁹F NMR (376 MHz, CDCl₃) δ: −147.26 (br d, J=19.1 Hz, 1F), −148.94 (br d, J=19.8 Hz, 1F).

### Example 38

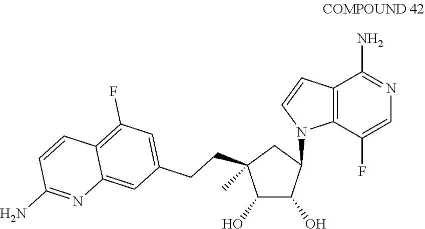 [SMILES: C[C@]1(CCc2cc(F)c3ccc(N)nc3c2)C[C@@H](n2ccc3c(N)ncc(F)c32)[C@H](O)[C@@H]1O]

(1S,2R,3S,5R)-3-(2-(2-amino-5-fluoroquinolin-7-yl)ethyl)-5-(4-amino-7-fluoro-1H-pyrrolo[3,2-c]pyridin-1-yl)-3-methylcyclopentane-1,2-diol (42) was obtained as hydrochloride salt and was prepared similarly as described for (1S,2R,3S,5R)-3-[2-(2-amino-3-chloro-5-fluoro-7-quinolyl)ethyl]-5-(4-amino-7-fluoro-pyrrolo[3,2-c]pyridin-1-yl)-3-methyl-cyclopentane-1,2-diol hydrochloride salt starting from 1-[(3aR,4R,6R,6aS)-2,2,4-trimethyl-4-vinyl-3a,5,6,6a-tetrahydrocyclopenta[d][1,3]dioxol-6-yl]-7-fluoro-pyrrolo[3,2-c]pyridin-4-amine (3Q) in a reaction with 7-bromo-3-chloro-5-fluoro-quinolin-2-amine (Q16) to afford the desired compound (106 mg, 0.199 mmol, 95%) as an off-white solid. LCMS: (ESI): RT=4.523 min, m/z calcd. for C₂₄H₂₆F₂O₂N₅ 454.2, [M+H]⁺, found 454.1. ¹H NMR (400 MHz, CD₃OD) δ: 8.43 (d, J=9.5 Hz, 1H), 7.74 (d, J=3.3 Hz, 1H), 7.60 (d, J=6.4 Hz, 1H), 7.37 (s, 1H), 7.24 (d, J=10.6 Hz, 1H), 7.13 (dd, J=2.0, 3.3 Hz, 1H), 7.06 (d, J=9.5 Hz, 1H), 5.19-5.10 (m, 1H), 4.47-4.39 (m, 1H), 3.93 (d, J=6.4 Hz, 1H), 2.99-2.79 (m, 2H), 2.16 (br dd, J=8.4, 12.8 Hz, 1H), 1.95-1.79 (m, 3H), 1.23 (s, 3H).

### Example 39

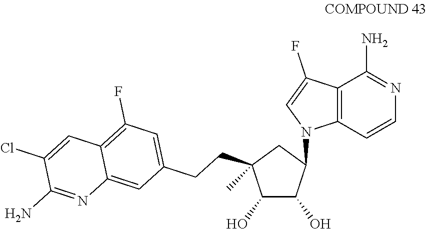 [SMILES: C[C@]1(CCc2cc(F)c3cc(Cl)c(N)nc3c2)C[C@@H](n2cc(F)c3c(N)nccc32)[C@H](O)[C@@H]1O]

(1S,2R,3S,5R)-3-(2-(2-amino-3-chloro-5-fluoroquinolin-7-yl)ethyl)-5-(4-amino-3-fluoro-1H-pyrrolo[3,2-c]pyridin-1-yl)-3-methylcyclopentane-1,2-diol (43) was obtained similar as described for (1S,2R,3S,5R)-5-(4-amino-1H-pyrrolo[3,2-c]pyridin-1-yl)-3-(2-(2-amino-3-chloro-5-fluoroquinolin-7-yl)ethyl)-3-methylcyclopentane-1,2-diol, using 4-chloro-3-fluoro-1H-pyrrolo[3,2-c]pyridine instead of 4-chloro-1H-pyrrolo[3,2-c]pyridine. The obtained residue was purified by prep-HPLC (column: Phenomenex Gemini-NX 80*40 mm*3 um; mobile phase: [water (0.05% NH₃H₂O+10 mM NH₄HCO₃)-ACN]; B %: 36%-62%, 8 min) to afford (1S,2R,3S,5R)-3-(2-(2-amino-3-chloro-5-fluoroquinolin-7-yl)ethyl)-5-(4-amino-3-fluoro-1H-pyrrolo[3,2-c]pyridin-1-yl)-3-methylcyclopentane-1,2-diol (65 mg, 0.132 mmol) as an off-white solid. ¹H NMR (400 MHz, CD₃OD) δ=8.19 (s, 1H), 7.54 (d, J=6.3 Hz, 1H), 7.24 (s, 1H), 7.17 (d, J=2.5 Hz, 1H), 6.93 (d, J=10.8 Hz, 1H), 6.82 (dd, J=2.5, 6.5 Hz, 1H), 4.73 (q, J=8.8 Hz, 1H), 4.30-4.23 (m, 1H), 3.86 (d, J=6.3 Hz, 1H), 2.89-2.71 (m, 2H), 2.12-2.02 (m, 1H), 1.93-1.73 (m, 3H), 1.22 (s, 3H). ¹⁹F NMR (376 MHz, CD₃OD-d₄) δ=−125.79 (s, 1F), −172.16 (s, 1F).

### Example 40

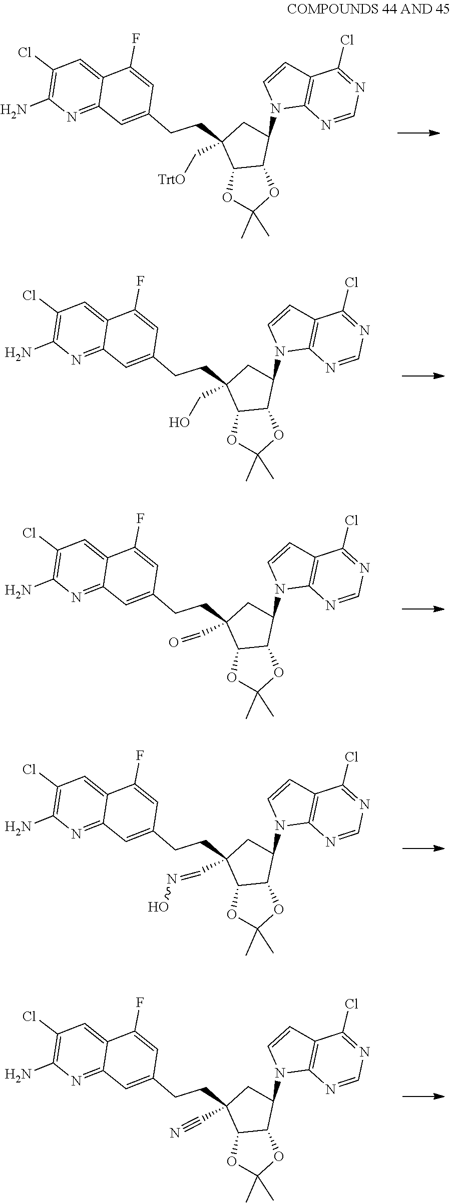

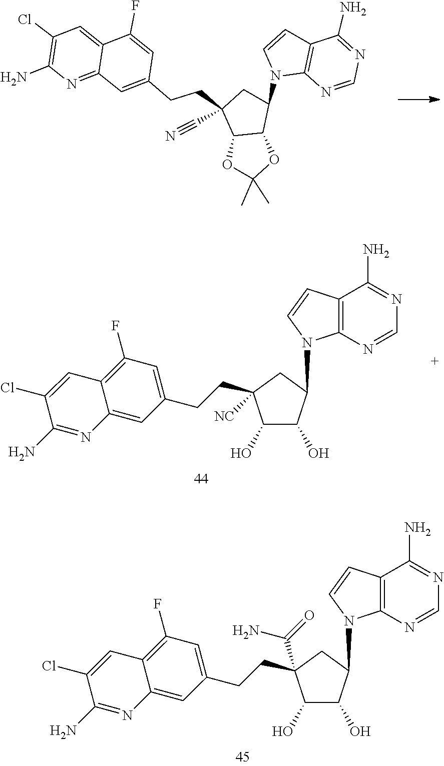 [SMILES: CC1(C)O[C@H]2[C@H](n3ccc4c(N)ncnc43)C[C@](C#N)(CCc3cc(F)c4cc(Cl)c(N)nc4c3)[C@H]2O1 ; N#C[C@]1(CCc2cc(F)c3cc(Cl)c(N)nc3c2)C[C@@H](n2ccc3c(N)ncnc32)[C@H](O)[C@@H]1O ; "+" ; NC(=O)[C@]1(CCc2cc(F)c3cc(Cl)c(N)nc3c2)C[C@@H](n2ccc3c(N)ncnc32)[C@H](O)[C@@H]1O]

To a solution of 3-chloro-7-(2-((3aR,4S,6R,6aS)-6-(4-chloro-7H-pyrrolo[2,3-d]pyrimidin-7-yl)-2,2-dimethyl-4-((trityloxy)methyl)tetrahydro-3aH-cyclopenta[d][1,3]dioxol-4-yl)ethyl)-5-fluoroquinolin-2-amine (200 mg, 0.254 mmol, 1 eq.) in DCM (25 mL) were added triethylsilane (3.64 g, 31.30 mmol, 5.00 mL, 123.4 eq.) and TFA (770.00 mg, 6.75 mmol, 0.5 mL, 26.63 eq.) in DCM (5 mL) at −20° C. The mixture was stirred at −20° C. for 5 min. Upon completion, the mixture was diluted with NH₄Cl (10 mL) and extracted with EtOAc (3×50 mL). The combined organic layers were washed with brine (150 mL), dried over anhydrous Na₂SO₄, filtered and concentrated under reduced pressure to give a residue, which was purified by flash silica gel chromatography (ISCO®; 24 g SepaFlash® Silica Flash Column, Eluent of 0˜4% MeOH/DCM @ 35 mL/min) to give ((3aR,4S,6R,6aS)-4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxol-4-yl)methanol (113 mg, 0.207 mmol, 82% yield) as a yellow solid. LCMS: (ESI): m/z calcd. for C₂₆H₂₇Cl₂FN₅O₃, 546.1 [M+H]⁺, found 546.1.

To a solution of ((3aR,4S,6R,6aS)-4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxol-4-yl)methanol (146 mg, 0.267 mmol, 1 eq.) in DMSO (1 mL) was added IBX (149.6 mg, 0.534 mmol, 2 eq.), and the mixture was stirred at 25° C. for 12 h. The reaction progress was monitored by TLC (DCM:MeOH=10:1). Upon completion, the mixture was diluted with H₂O (10 mL) and extracted with EtOAc (3×20 mL, and concentrated under reduced pressure to give (3aR,4R,6R,6aS)-4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxole-4-carbaldehyde (150 mg, crude) as a colorless oil, which was used for next step without further purification. LCMS: (ESI): m/z calcd. for C₂₆H₂₅Cl₂FN₅O₃ 544.1. [M+H]⁺, found 544.1.

To a solution of (3aR,4R,6R,6aS)-4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxole-4-carbaldehyde (100 mg, 183.69 μmol, 1 eq.) in EtOH (1.6 mL) and H₂O (0.2 mL) were added NaOAc (30.14 mg, 367.37 μmol, 2 eq.) and NH₂OH.HCl (25.53 mg, 367.37 μmol, 2 eq.). The mixture was stirred at 25° C. for 3 h. The reaction progress was monitored by TLC (DCM:MeOH=10:1). Upon completion, the mixture was diluted with NH₄Cl (10 mL) and extracted with EtOAc (3×50 mL). The combined organic layers were washed with brine (50 mL), dried over anhydrous Na₂SO₄, filtered and concentrated under reduced pressure to afford 4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxole-4-carbaldehyde oxime (93 mg, crude) as a white solid, which was used for next step without further purification. LCMS: (ESI): m/z calcd. for C₂₆H₂₆Cl₂FN₆O₃ 559.1. [M+H]⁺, found 559.2.

To a solution of 4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxole-4-carbaldehyde oxime (80 mg, 0.143 mmol, 1 eq.) in CH₃CN (4 mL) were added CDI (115.9 mg, 0.715 mmol, 5 eq.) and Et₃N (72.4 mg, 0.715 mmol, 5 eq.). The mixture was stirred at 25° C. for 4 h. The reaction progress was monitored by TLC (DCM:MeOH=10:1). Upon completion, the residue was diluted with H₂O (20 mL) and extracted with EtOAc (3×30 mL). The combined organic layers were washed with brine (90 mL), dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜8% EA:PE gradient @ 20 mL/min) to give (3aR,4S,6R,6aS)-4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxole-4-carbonitrile (61.4 mg, 0.110 mmol, 77% yield,) as a white solid. LCMS: (ESI): m/z calcd. for C₂₆H₂₄N₆O₂Cl₂F, 541.1 [M+H]⁺, found 541.0.

To a solution of (3aR,4S,6R,6aS)-4-(2-(2-amino-3-chloro-5-fluoroquinolin-7-yl)ethyl)-6-(4-chloro-7H-pyrrolo[2,3-d]pyrimidin-7-yl)-2,2-dimethyltetrahydro-4H-cyclopenta[d][1,3]dioxole-4-carbonitrile (85 mg, 0.157 mmol, 1 eq.) in dioxane (5 mL) and NH₃.H₂O (5 mL, 25% wt) was stirred at 110° C. for 12 h in a 30 mL sealed tube. Upon completion, the mixture was concentrated. The residue was diluted with NaHCO₃ (sat. aq., 5 mL) and extracted with EtOAc (3×50 mL). The combined organic layers were washed with brine (150 mL), dried over anhydrous Na₂SO₄, filtered, and concentrated under reduced pressure to give a residue, which was purified by flash silica gel chromatography (ISCO®; 25 g SepaFlash® Silica Flash Column, Eluent of 0˜4.7% EA:PE gradient @ 35 mL/min) to give (3aR,4S,6R,6aS)-4-(2-(2-amino-3-chloro-5-fluoroquinolin-7-yl)ethyl)-6-(4-amino-7H-pyrrolo[2,3-d]pyrimidin-7-yl)-2,2-dimethyltetrahydro-4H-cyclopenta[d][1,3]dioxole-4-carbonitrile (66 mg, 0.126 mmol, 81% yield) as a white solid. LCMS: (ESI): m/z calcd. for C₂₆H₂₆ClFN₇O₂, 522.2 [M+H]⁺, found 522.1.

To a solution of (3aR,4S,6R,6aS)-4-(2-(2-amino-3-chloro-5-fluoroquinolin-7-yl)ethyl)-6-(4-amino-7H-pyrrolo[2,3-d]pyrimidin-7-yl)-2,2-dimethyltetrahydro-4H-cyclopenta[d][1,3]dioxole-4-carbonitrile (66 mg, 0.126 mmol, 1 eq.) in THF (2 mL) was added 4 M HCl (1 mL). The mixture was stirred at 25° C. for 15 h. Upon completion, the mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 21%-45%, 8 min) to give (1S,2R,3S,4R)-1-(2-(2-amino-3-chloro-5-fluoroquinolin-7-yl)ethyl)-4-(4-amino-7H-pyrrolo[2,3-d]pyrimidin-7-yl)-2,3-dihydroxycyclopentane-1-carbonitrile (44) (32 mg, 0.066 μmol, 52% yield, 99% purity) and (1R,2R,3S,4R)-1-(2-(2-amino-3-chloro-5-fluoroquinolin-7-yl)ethyl)-4-(4-amino-7H-pyrrolo[2,3-d]pyrimidin-7-yl)-2,3-dihydroxycyclopentane-1-carboxamide (45) (5 mg, 0.010 μmol, 8% yield, 98% purity).

Compound 44: LCMS: (ESI): m/z calcd. for C₂₃H₂₂ClFN₇O₂ 482.1. [M+H]⁺, found 482.2. ¹H NMR (400 MHz, CD₃OD) δ: 8.20 (s, 1H), 8.09 (s, 1H), 7.30 (s, 1H), 7.21 (d, J=3.6 Hz, 1H), 6.97 (d, J=10.8 Hz, 1H), 6.58 (d, J=3.6 Hz, 1H), 5.07-4.99 (m, 1H), 4.64 (dd, J=6.3, 13.5 Hz, 1H), 4.15 (d, J=5.7 Hz, 1H), 3.10-2.91 (m, 2H), 2.72 (dd, J=9.0, 13.6 Hz, 1H), 2.37 (dd, J=10.3, 13.6 Hz, 1H), 2.32-2.23 (m, 2H). ¹⁹F NMR (376 MHz, CD₃OD) δ: −125.35 (s, 1F).

Compound 45: LCMS: (ESI): m/z calcd. for C₂₃H₂₄ClFN₇O₃ 500.2. [M+H]⁺, found 500.2. ¹H NMR (400 MHz, CD₃OD) δ: 8.19 (s, 1H), 8.10 (s, 1H), 7.26 (s, 1H), 7.21 (d, J=3.5 Hz, 1H), 6.93 (d, J=10.8 Hz, 1H), 6.59 (d, J=3.5 Hz, 1H), 4.96-4.92 (m, 1H), 4.72 (dd, J=4.9, 7.5 Hz, 1H), 4.19 (d, J=4.6 Hz, 1H), 3.01 (dd, J=9.9, 13.6 Hz, 1H), 2.81-2.65 (m, 2H), 2.32-2.22 (m, 2H), 2.06 (dd, J=9.3, 13.6 Hz, 1H). ¹⁹F NMR (376 MHz, CD₃OD) δ: −125.75 (s, 1F).

### Example 41

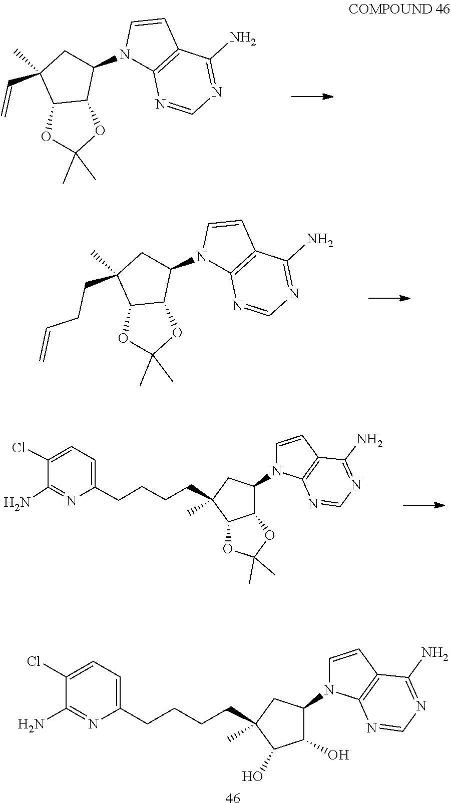

To a solution of 7-((3aS,4R,6R,6aR)-2,2,6-trimethyl-6-vinyltetrahydro-4H-cyclopenta[d][1,3]dioxol-4-yl)-7H-pyrrolo[2,3-d]pyrimidin-4-amine (500 mg, 1.59 mmol, 1 eq.) in THF (4 mL) was added 9-BBN dimer (962.3 mg, 3.98 mmol, 2.5 eq.). The mixture was stirred at 50° C. for 2 h under N₂, and then cooled to rt. A solution of K₃PO₄ (1.69 g, 7.95 mmol, 5 eq.) in H₂O (2 mL) was added. The mixture was stirred at rt for 0.5 h, and then vinyl bromide (1 M, 7 mL, 4.40 eq.) and Pd(dppf)Cl₂ (116.37 mg, 0.159 mmol, 0.1 eq.) were added. The mixture was stirred at 40° C. for 16 h under N₂. The mixture was then diluted with brine (10 mL) and extracted with EA (2×20 mL). The combined organic layers were combined, dried over Na₂SO₄, filtered and concentrated to afford a residue, which was purified by prep-HPLC (40 g C-18 column, gradient: 0%˜70% CH₃CN in water (1 mL NH₃.H₂O in 2 L H₂O) in 15 min@ 40 mL/min to give 7-((3aS,4R,6S,6aR)-6-(but-3-en-1-yl)-2,2,6-trimethyltetrahydro-4H-cyclopenta[d][1,3]dioxol-4-yl)-7H-pyrrolo[2,3-d]pyrimidin-4-amine (142.8 mg, 0.417 mmol, 26% yield) as a brown solid. LCMS: (ESI): RT=5.117 min, m/z calcd. for C₁₉H₂₇O₂N₄ 343.2, [M+H]⁺, found 343.3.

To a solution of 7-((3aS,4R,6S,6aR)-6-(but-3-en-1-yl)-2,2,6-trimethyltetrahydro-4H-cyclopenta[d][1,3]dioxol-4-yl)-7H-pyrrolo[2,3-d]pyrimidin-4-amine (142.8 mg, 0.417 mmol, 1 eq.) in THF (4 mL) was added 9-BBN dimer (252.31 mg, 1.04 mmol, 2.5 eq.). The mixture was stirred at 50° C. for 2 h under N₂, and then cooled to 25° C. A solution of K₃PO₄ (442.59 mg, 2.09 mmol, 5 eq.) in H₂O (0.4 mL) was added, and the mixture was then stirred at rt for 0.5 h. 6-bromo-3-chloro-pyridin-2-amine (103.8 mg, 0.500 mmol, 1.2 eq.) and Pd(dppf)Cl₂ (30.5 mg, 0.042 mmol, 0.1 eq.) were added, and the mixture was stirred at 60° C. for 16 h. The mixture was diluted with water (5 mL) and extracted with EA (3×10 mL). The combined organic layers were washed with brine (30 mL) (3×10 mL), dried over Na₂SO₄, filtered and concentrated to give 7-((3aS,4R,6S,6aR)-6-(4-(6-amino-5-chloropyridin-2-yl)butyl)-2,2,6-trimethyltetrahydro-4H-cyclopenta[d][1,3]dioxol-4-yl)-7H-pyrrolo[2,3-d]pyrimidin-4-amine (50.5 mg, 70% purity, 0.075 mmol, 18% yield). LCMS: (ESI): RT=1.757 min, m/z calcd. for C₂₄H₃₂ClO₂N₆ 471.22, [M+H]⁺, found 471.3.

To a solution of 7-((3aS,4R,6S,6aR)-6-(4-(6-amino-5-chloropyridin-2-yl)butyl)-2,2,6-trimethyltetrahydro-4H-cyclopenta[d][1,3]dioxol-4-yl)-7H-pyrrolo[2,3-d]pyrimidin-4-amine (50.5 mg, 70% purity, 0.075 mmol, 1 eq.) in THF (3 mL) was added HCl (4 M, 1.5 mL). The mixture was stirred at 20° C. for 12 h. Upon completion, the mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 20%-50%, 8 min) to give (1S,2R,3S,5R)-3-(4-(6-amino-5-chloropyridin-2-yl)butyl)-5-(4-amino-7H-pyrrolo[2,3-d]pyrimidin-7-yl)-3-methylcyclopentane-1,2-diol (46) (9.0 mg, 0.021 mmol, 28%). ¹H NMR (400 MHz, CD₃OD) δ: 8.05 (s, 1H), 7.45 (d, J=7.8 Hz, 1H), 7.21 (d, J=3.5 Hz, 1H), 6.60 (d, J=3.5 Hz, 1H), 6.51 (d, J=7.8 Hz, 1H), 5.01-4.93 (m, 1H), 4.45 (dd, J=6.3, 7.8 Hz, 1H), 3.80 (d, J=6.0 Hz, 1H), 2.64-2.57 (m, 2H), 1.98 (dd, J=8.8, 12.8 Hz, 1H), 1.77 (dd, J=10.8, 13.1 Hz, 1H), 1.68 (quin, J=7.3 Hz, 2H), 1.60-1.31 (m, 4H), 1.09 (s, 3H).

### Example 42

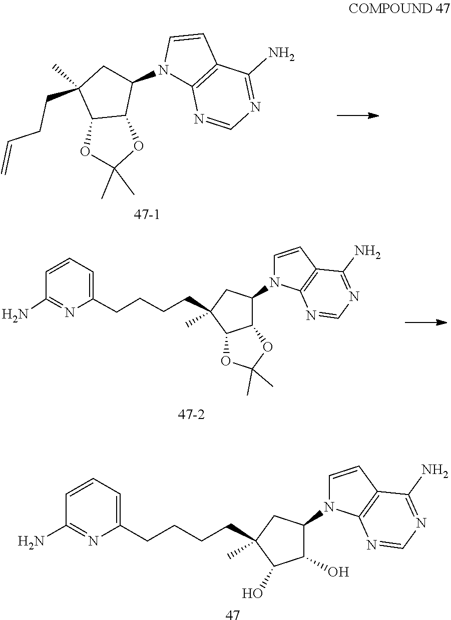

To a solution of 47-1 (177.5 mg, 0.508 mmol, 1 eq.) in THF (4 mL) was added 9-BBN dimer (313.6 mg, 1.30 mmol, 2.5 eq.), and the mixture was stirred at 50° C. for 2 h under N₂. The mixture was cooled to rt, and then a solution of K₃PO₄ (550.1 mg, 2.6 mmol, 5 eq.) in H₂O (0.5 mL) was added. After stirring at rt for 0.5 h, 6-bromopyridin-2-amine (107.6 mg, 0.622 mmol, 1.2 eq.) and Pd(dppf)Cl₂ (37.9 mg, 0.052 mmol, 0.1 eq.) were added, and the mixture was stirred at 60° C. for 16 h under N₂. Upon completion, the mixture was diluted with brine (10 mL) and extracted with EA (2×20 mL). The combined organic layers were combined and dried over NaS₂O₄, filtered, and concentrated to give a residue. The residue was purified by prep-HPLC (40 g C-18 column, gradient: 0%˜70% CH₃CN in water (1 mL NH₃.H₂O in 2 L H₂O) in 15 min@ 40 mL/min to afford 47-2 (85.4 mg, 0.196 mmol, 38% yield) as a brown solid. LCMS: (ESI): RT=1.553 min, m/z calcd. for C₂₄H₃₃O₂N₆ 437.26, [M+H]⁺, found 437.2.

To a solution of 47-2 (85.4 mg, 0.196 mmol, 1 eq.) in THF (3 mL) was added HCl (aq., 4 M, 1.5 mL), and the mixture was stirred at 20° C. for 12 h. Upon completion, the mixture was concentrated under reduced pressure to give a residue, which was purified by prep-HPLC (column: Venusil ASB Phenyl 150*30 mm*5 um; mobile phase: [water (0.05% HCl)-ACN]; B %: 9%-39%, 9 min) to afford 47 as a hydrochloride salt (54.4 mg, 0.115 mmol, 59% yield). LCMS: (ESI): RT=3.688 min, m/z calcd. for C₂₁H₂₉N₆O₂ 397.23, [M+H]⁺, found 397.3. ¹H NMR (400 MHz, CD₃OD) δ: 8.25 (s, 1H), 7.84 (dd, J=7.3, 8.8 Hz, 1H), 7.57 (d, J=3.5 Hz, 1H), 6.92 (d, J=3.5 Hz, 1H), 6.84 (d, J=8.8 Hz, 1H), 6.76 (d, J=7.3 Hz, 1H), 5.14-5.05 (m, 1H), 4.55-4.48 (m, 1H), 3.84 (d, J=6.0 Hz, 1H), 2.80 (br t, J=7.8 Hz, 2H), 2.04-1.96 (m, 1H), 1.92-1.83 (m, 1H), 1.81-1.72 (m, 2H), 1.66-1.38 (m, 4H), 1.11 (s, 3H).

### Example 43

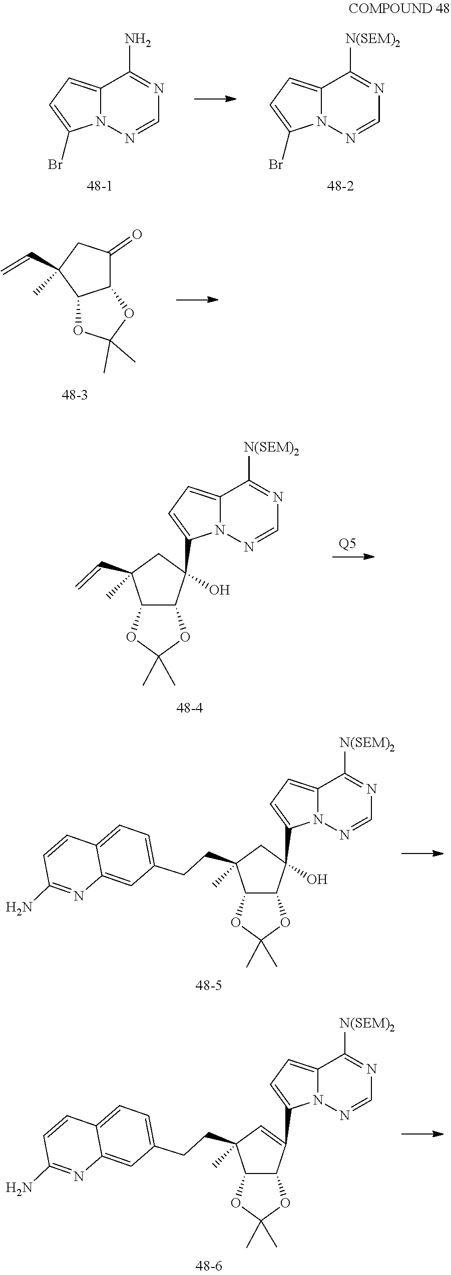

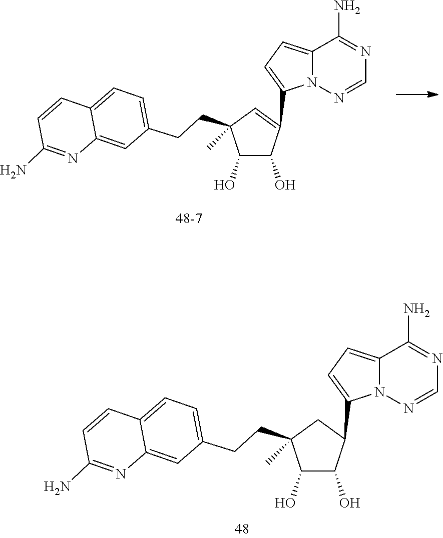

To a solution of 7-bromopyrrolo[2,1-f][1,2,4]triazin-4-amine (48-1) (5.5 g, 25.82 mmol, 1 eq.) in DMF (50 mL) was added NaH (2.58 g, 64.54 mmol, 60% purity, 2.5 eq.) at 0° C., and the mixture was stirred at 0° C. for 0.5 h. 2-(chloromethoxy) ethyl-trimethyl-silane (9.04 g, 54.22 mmol, 9.60 mL, 2.1 eq.) was added, and the mixture was stirred at 25° C. for 5 h. Upon completion, the reaction was quenched by sat. aq. NH₄Cl (50 mL) and extracted with EA (2×50 mL). The combined organic layers were washed with brine (50 mL), dried over Na₂SO₄, filtered, and concentrated under reduced pressure to give a residue, which was purified by flash silica gel chromatography (ISCO®; 40 g SepaFlash® Silica Flash Column, Eluent of 0˜2% EA/PE gradient @ 40 mL/min) to afford 48-2 (7.6 g, 15.90 mmol, 61% yield) as a colorless oil. LCMS: (ESI): m/z calcd. for C₁₈H₃₄BrN₄O₂Si₂ 473.13 [M+H]⁺, found 473.2. ¹H NMR (400 MHz, CDCl₃) δ: 8.09 (s, 1H), 7.08 (d, J=4.8 Hz, 1H), 6.77 (d, J=4.8 Hz, 1H), 5.22 (s, 4H), 3.70-3.65 (m, 4H), 1.02-0.95 (m, 4H), 0.02-0.00 (m, 18H).

Chloro(isopropyl)magnesium lithium chloride (1.3 M, 5.88 mL in THF, 3 eq.) was added dropwise to a solution of 48-2 (3.62 g, 7.64 mmol, 3 eq.) in THF (5 mL) at −20° C. The mixture was stirred at −20° C. for 10 min, then warmed to 0° C. The mixture was stirred at 0° C. for 1 h, and a solution of 48-3 (500 mg, 2.55 mmol, 1 eq.) in THF (7 mL) was added dropwise at −20° C. The mixture was stirred at 0° C. for 1 h. Upon completion, the reaction was quenched by sat. NH₄Cl solution (15 mL) and extracted with EA (2×20 mL). The combined organic layers were washed with water and brine (20 mL), dried over Na₂SO₄, filtered and concentrated under reduced pressure to give a residue, which was purified by flash silica gel chromatography (ISCO®; 40 g SepaFlash® Silica Flash Column, Eluent of 0˜10% EA/PE gradient @ 30 mL/min) to give 48-4 (967 mg, 94% purity, 1.54 mmol, 60% yield) as a yellow oil. LCMS: (ESI): m/z calcd. for C₂₉H₅₁N₄O₅Si₂ 591.33 [M+H]⁺, found 591.4.

A mixture of 48-4 (470 mg, 0.795 mmol, 1 eq.) and 9-BBN dimer (481.2 mg, 1.99 mmol, 2.5 eq.) in THF (10 mL) was stirred at 50° C. for 2 h under N₂. The mixture was cooled to rt, and a solution of K₃PO₄ (844.2 mg, 3.98 mmol, 5 eq.) in H₂O (1 mL) was added. The mixture was stirred for 0.5 h. Q5 (212.9 mg, 0.954 mmol, 1.2 eq.) and Pd(dppf)Cl₂ (58.20 mg, 0.080 mmol, 0.1 eq.) were added. The mixture was stirred at 70° C. under N₂ for 12 h. The mixture was diluted with brine (10 mL) and extracted with EA (2×10 mL). The combined organic layers were dried over Na₂SO₄, filtered and concentrated under reduced pressure to give a residue. The residue was purified by column chromatography (SiO₂, PE:EA (3:1 to 1:1) to (DCM:MeOH 30:1 to 20:1)) to afford 48-5 (508 mg, 0.580 mmol, 72% yield) as a yellow solid. LCMS: (ESI): m/z calcd. for C₃₈H₅₉N₆O₅Si₂ 735.4 [M+H]⁺, found 735.5.

To a solution of 48-5 (350 mg, 0.476 mmol, 1 eq.) in DCM (8.5 mL) was added DAST (383.8 mg, 2.38 mmol, 314 μL, 5 eq.) at 0° C. The mixture was stirred at 0° C. for 1 h. The mixture was filtered and concentrated under reduced pressure to give a residue. The residue was purified by column chromatography (SiO₂, DCM:MeOH (50:1 to 20:1)) to afford 48-6 (313 mg, 0.393 mmol, 82% yield) as a white solid. LCMS: (ESI): m/z calcd. for C₃₈H₅₇N₆O₄Si₂ 717.39 [M+H]⁺, found 717.4.

To a solution of 48-6 (313 mg, 0.393 mmol, 1 eq.) in THF (8.5 mL) was added HCl (aq., 4 M, 4 mL). The mixture was stirred at 25° C. for 3.5 h. The mixture was filtered and concentrated under reduced pressure to give a residue. To a solution of the residue in a mixed a solvent of t-BuOH (7 mL) and H₂O (3 mL) was added PPTS (700.71 mg, 2.79 mmol, 5 eq.). The mixture was stirred at 50° C. for 12 h, and then concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 17%-47%, 8 min) to afford 48-7 (57 mg, 0.133 mmol, 34% yield) as a white solid. LCMS: (ESI): m/z calcd. for C₂₃H₂₃N₆O₂ 417.2 [M+H]⁺, found 417.3.

A mixture of 48-7 (48 mg, 0.115 mmol, 1 eq.) and PtO₂ (250 mg, 1.10 mmol, 9.55 eq.) in THF (10 mL) was hydrogenated under H₂ (1 atm) at rt for 12 h. The mixture was filtered through a pad of Celite. The filtrate was concentrated under reduced pressure to give a residue, which was purified by prep-HPLC (column: Phenomenex Gemini-NX 80*40 mm*3 um; mobile phase: [water (0.05% NH₃H₂O+10 mM NH₄HCO₃)-ACN]; B %: 24%-38%, 8 min) to give 48 (10 mg, 0.0236 mmol, 20.5% yield, 98.88% purity) as a white solid. LCMS: (ESI): m/z calcd. for C₂₃H₂₇N₆O₂ 419.21 [M+H]⁺, found 419.3. ¹H NMR (400 MHz, CD₃OD) δ: 7.90 (d, J=8.8 Hz, 1H), 7.75 (s, 1H), 7.56 (d, J=8.3 Hz, 1H), 7.35 (s, 1H), 7.12 (dd, J=1.5, 8.0 Hz, 1H), 6.84 (d, J=4.5 Hz, 1H), 6.76 (d, J=8.8 Hz, 1H), 6.55 (d, J=4.5 Hz, 1H), 4.46-4.41 (m, 1H), 3.86 (d, J=6.0 Hz, 1H), 3.78-3.70 (m, 1H), 2.88-2.69 (m, 2H), 2.08-2.01 (m, 1H), 1.89-1.66 (m, 3H), 1.23 (s, 3H).

### Example 44

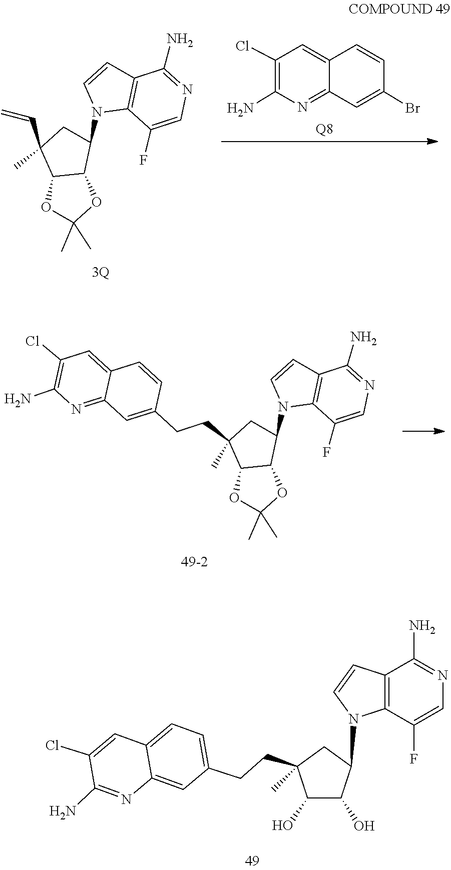

To a mixture of 3Q (100 mg, 0.302 mmol, 1 eq.) in THF (5 mL) was added 9-BBN dimer (160.7 mg, 0.664 mmol, 2.2 eq.). The mixture was stirred at 50° C. for 1 h and then cooled to rt. A solution of K₃PO₄ (320.3 mg, 1.51 mmol, 5 eq.) in H₂O (0.5 mL) was added, and the mixture was stirred at rt for 0.2 h. 7-bromo-3-chloro-quinolin-2-amine Q8 (93.3 mg, 0.362 mmol, 1.2 eq.) and Pd(dppf)Cl₂ (22.08 mg, 0.030 mmol, 0.1 eq.) were then added. The mixture was stirred at 60° C. for 8 h. Upon completion, the mixture was diluted with brine (10 mL) and extracted with EA (3×20 mL). The separated organic layers were combined, dried over anhydrous Na₂SO₄ and concentrated to give a residue. The residue was purified by prep-HPLC (40 g C-18 column: chromatography (0%˜65% CH₃CN/H₂O (1 mL NH₃.H₂O in 2 L H₂O) @ 40 mL/min) to afford 49-2 (55 mg, 0.105 mmol, 35% yield, 97.4% purity) as a brown solid. LCMS: (ESI): m/z calcd. for C₂₇H₃₀ClFN₅O₂ 510.20 [M+H]⁺, found 510.3.

To a solution of 49-2 (55 mg, 0.105 mmol, 1 eq.) in THF (3 mL) was added HCl (4 M, 2 mL). The mixture was stirred at rt for 6 h. Upon completion, the mixture was concentrated under reduced pressure to afford a residue, which was purified by prep-HPLC twice (column: Phenomenex Gemini-NX 80*40 mm*3 um; mobile phase: [water (0.05% NH₃H₂O+10 mM NH₄HCO₃)-ACN]; B %: 32%-52%, 8 min) to give 49 (28 mg, 0.0593 mmol, 56% yield, 99.49% purity) as a white solid. LCMS: (ESI): m/z calcd. for C₂₄H₂₆ClFN₅O₂ 470.17 [M+H]⁺, found 470.1. ¹H NMR (400 MHz, CD₃OD) δ: 8.06 (s, 1H), 7.56 (d, J=8.3 Hz, 1H), 7.42 (d, J=5.0 Hz, 1H), 7.40 (s, 1H), 7.35 (d, J=3.3 Hz, 1H), 7.17 (dd, J=1.5, 8.3 Hz, 1H), 6.73-6.68 (m, 1H), 5.11-5.00 (m, 1H), 4.46-4.38 (m, 1H), 3.91 (d, J=6.5 Hz, 1H), 2.90-2.69 (m, 2H), 2.11 (dd, J=8.3, 12.8 Hz, 1H), 1.93-1.73 (m, 3H), 1.22 (s, 3H).

### Example 45

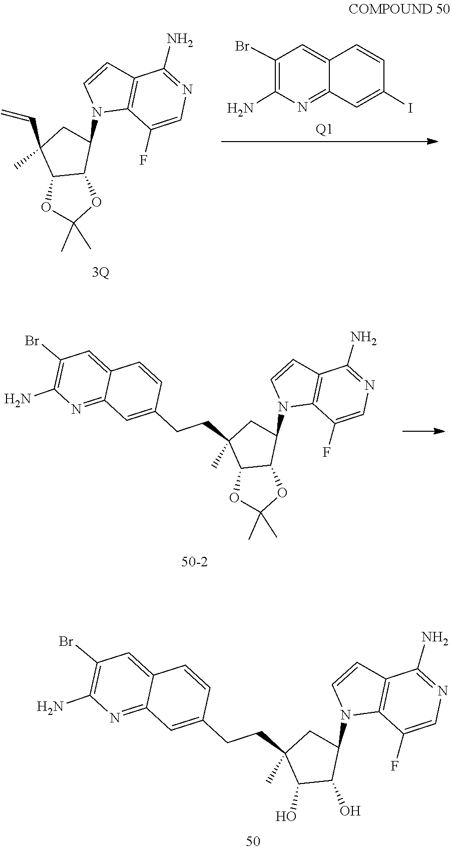

To a solution of 3Q (100 mg, 0.302 mmol, 1 eq.) in THF (5 mL) was added 9-BBN dimmer (160.7 mg, 0.664 mmol, 2.2 eq.). The mixture was stirred at 50° C. for 1 h and cooled to rt. A solution of K₃PO₄ (320.3 mg, 1.51 mmol, 5 eq.) in H₂O (0.5 mL) was added, and the mixture was stirred at rt for 0.2 h. 3-bromo-7-iodo-quinolin-2-amine (Q1) (126.4 mg, 0.362 mmol, 1.2 eq.) and Pd(dppf)Cl₂ (22.1 mg, 0.030 mmol, 0.1 eq.) were then added. The mixture was stirred at 50° C. for 3 h. Upon completion, the mixture was diluted with brine (10 mL) and extracted with EA (3×20 mL). The separated organic layers were combined, dried over anhydrous Na₂SO₄ and concentrated to give a residue, which was purified by column chromatography (100-200 mesh, SiO₂, CH₂Cl₂/MeOH (100:1 to 10:1), eluted 1 L) to provide 50-2 (160 mg, 0.212 mmol, 73.4% purity, 70% yield) as a brown solid. LCMS: (ESI): m/z calcd. for C₂₇H₃₀BrFN₅O₂ 554.15 [M+H]⁺, found 556.1.

To a solution of 50-2 (160 mg, 0.212 mmol, 1 eq.) in THF (4 mL) was added HCl (4 M, 2 mL). The mixture was stirred at rt for 8 h. Upon completion, the mixture was concentrated under reduced pressure to afford a residue. The residue was purified by prep-HPLC (acidic condition, column: Agela ASB 150*25 mm*5 um; mobile phase: [water (0.05% HCl)-ACN]; B %: 12%-42%, 9 min), and then (basic condition, column: Phenomenex Gemini-NX 80*40 mm*3 um; mobile phase: [water (0.05% NH₃H₂O+10 mM NH₄HCO₃)-ACN]; B %: 30%-60%, 8 min) to give 50 (25 mg, 0.0484 mmol, 23% yield, 99.66% purity) as a white solid. LCMS: (ESI): m/z calcd. for C₂₄H₂₆BrFN₅O₂ 516.12 [M+H]⁺, found 516.1. ¹H NMR (400 MHz, CD₃OD) δ: 8.25 (s, 1H), 7.55 (d, J=8.3 Hz, 1H), 7.42 (d, J=5.0 Hz, 1H), 7.39 (s, 1H), 7.35 (d, J=3.3 Hz, 1H), 7.17 (dd, J=1.5, 8.3 Hz, 1H), 6.73-6.67 (m, 1H), 5.12-5.01 (m, 1H), 4.45-4.39 (m, 1H), 3.91 (d, J=6.3 Hz, 1H), 2.90-2.69 (m, 2H), 2.11 (dd, J=8.8, 12.5 Hz, 1H), 1.93-1.72 (m, 3H), 1.22 (s, 3H). ¹⁹F NMR (376 MHz, CD₃OD) δ: −161.68 (br s, 1F).

### Example 46

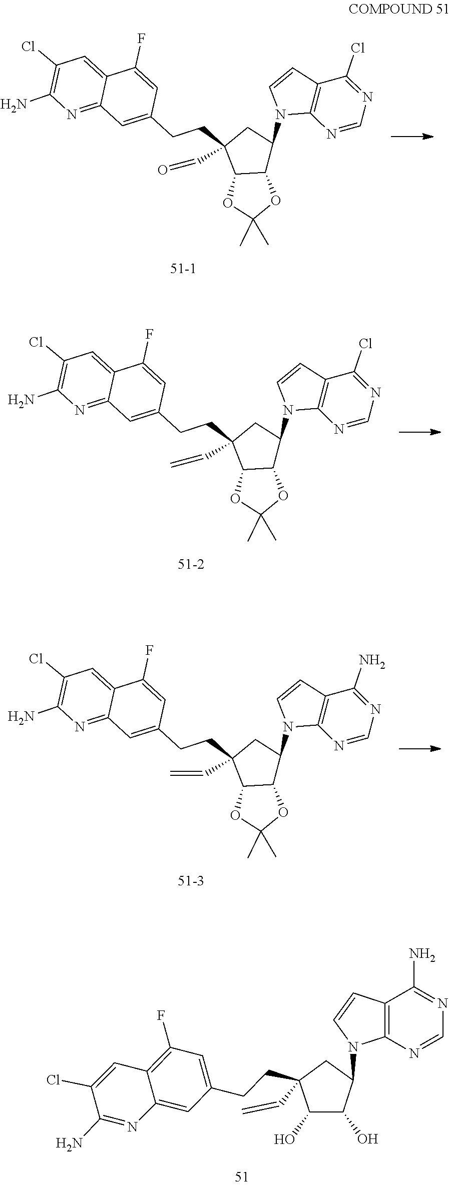 [SMILES: CC1(C)O[C@H]2[C@H](n3ccc4c(Cl)ncnc43)C[C@](C=O)(CCc3cc(F)c4cc(Cl)c(N)nc4c3)[C@H]2O1 ; C=C[C@]1(CCc2cc(F)c3cc(Cl)c(N)nc3c2)C[C@@H](n2ccc3c(Cl)ncnc32)[C@@H]2OC(C)(C)O[C@@H]21 ; C=C[C@]1(CCc2cc(F)c3cc(Cl)c(N)nc3c2)C[C@@H](n2ccc3c(N)ncnc32)[C@@H]2OC(C)(C)O[C@@H]21 ; C=C[C@]1(CCc2cc(F)c3cc(Cl)c(N)nc3c2)C[C@@H](n2ccc3c(N)ncnc32)[C@H](O)[C@@H]1O]

To a mixture of PPh₃CH₃Br (255.91 mg, 716.38 μmol, 5.2 eq.) in THF (3 mL) was added t-BuOK (77.29 mg, 688.83 μmol, 5 eq.) at 0° C., and the mixture was stirred at 25° C. for 0.5 h. Compound 51-1 (75 mg, 137.77 μmol, 1 eq.) was added, and the mixture was stirred at 25° C. for 1.5 h. Upon completion, the reaction was quenched by the addition of NH₄Cl (10 mL) at 0° C. The mixture was then extracted with EtOAc (3×20 mL). The combined organic layers were washed with brine (3×20 mL), dried over anhydrous Na₂SO₄, filtered and concentrated under reduced pressure to give a residue. The residue was purified by flash silica gel chromatography (ISCO®; 20 g SepaFlash® Silica Flash Column, Eluent of 0˜1.9% MeOH/DCM @ 30 mF/min) to afford 51-2 (38 mg, 64.45 μmol, 46.78% yield, 88% purity) as a colorless oil. LCMS: (ESI): m/z calcd. for C₂₇H₂₇Cl₂FN₅O₂ 542.1. [M+H]⁺, found 542.1.

A solution of 51-2 (38 mg, 70.05 μmol, 1 eq.) in dioxane (1 mL) and NH₃.H₂O (4.55 g, 32.46 mmol, 5 mL, 25% aq., 206.74 eq.) was stirred in a 30 mL of sealed tube at 110° C. for 24 h. Upon completion, the reaction was quenched by the addition of NaHCO₃ (sat. aq., 5 mL). The mixture was extracted with EtOAc (3×20 mL). The combined organic layers were washed with brine (3×20 mL), dried over anhydrous Na₂SO₄, filtered and concentrated under reduced pressure to give a residue. The residue was purified by flash silica gel chromatography (ISCO®; 25 g SepaFlash® Silica Flash Column, Eluent of 0˜3.1% MeOH/DCM gradient @ 30 mL/min) to afford 51-3 (26 mg, 46.73 μmol, 66.71% yield, 94% purity) as a white solid. LCMS: (ESI): m/z calcd. for C₂₇H₂₉ClFN₆O₂, 523.2 [M+H]+, found 523.1.

To a solution of 51-3 (26 mg, 49.71 μmol, 1 eq.) in THF (1 mL) was added HCl (4M, aq., 1 mL), and the mixture was stirred at 25° C. for 12 h. Upon completion, the mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 21%-45%, 8 min) to afford 51 (15 mg, 31.06 μmol, 62.48% yield, 100% purity) as a white solid. LCMS: (ESI): m/z calcd. for C₂₄H₂₅ClFN₆O₂ 483.2. [M+H]⁺, found 483.3. ¹H NMR (400 MHz, CD₃OD) δ: 8.18 (s, 1H), 8.09 (s, 1H), 7.23 (d, J=3.5 Hz, 1H), 7.21 (s, 1H), 6.89 (d, J=10.8 Hz, 1H), 6.59 (d, J=3.5 Hz, 1H), 6.13 (dd, J=11.0, 17.8 Hz, 1H), 5.42-5.22 (m, 2H), 5.03-4.92 (m, 1H), 4.60 (d, J=6.5 Hz, 1H), 4.02 (d, J=6.0 Hz, 1H), 2.81-2.61 (m, 2H), 2.57 (dd, J=9.0, 13.3 Hz, 1H), 2.11-1.92 (m, 3H). ¹⁹F NMR (376 MHz, CD₃OD) δ: −125.86 (s, 1F).

### Example 47

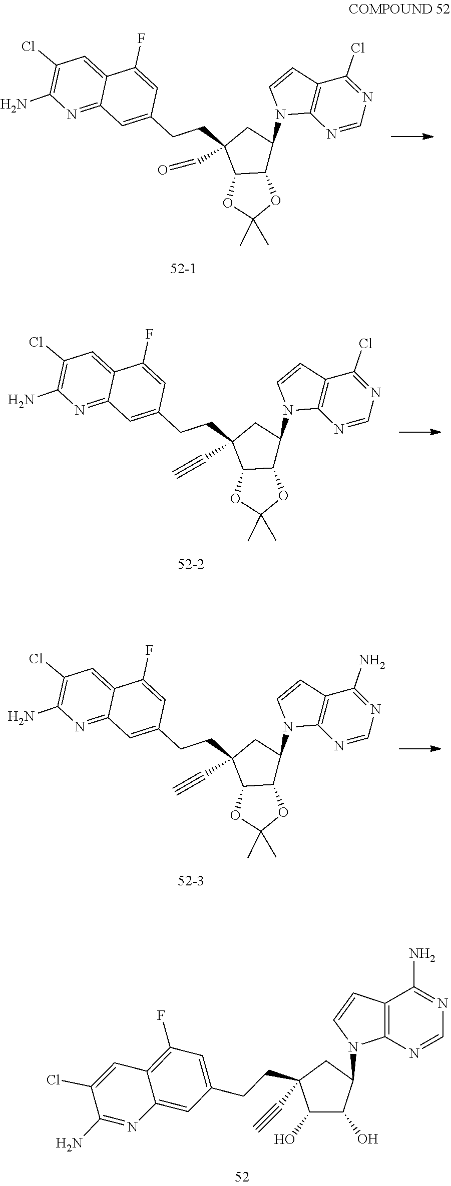 [SMILES: CC1(C)O[C@H]2[C@H](n3ccc4c(Cl)ncnc43)C[C@](C=O)(CCc3cc(F)c4cc(Cl)c(N)nc4c3)[C@H]2O1 ; C#C[C@]1(CCc2cc(F)c3cc(Cl)c(N)nc3c2)C[C@@H](n2ccc3c(Cl)ncnc32)[C@@H]2OC(C)(C)O[C@@H]21 ; C#C[C@]1(CCc2cc(F)c3cc(Cl)c(N)nc3c2)C[C@@H](n2ccc3c(N)ncnc32)[C@@H]2OC(C)(C)O[C@@H]21 ; C#C[C@]1(CCc2cc(F)c3cc(Cl)c(N)nc3c2)C[C@@H](n2ccc3c(N)ncnc32)[C@H](O)[C@@H]1O]

To a solution of 52-1 (75 mg, 137.77 μmol, 1 eq.) in CH₃CN (3 mL) were added K₂CO₃ (38.08 mg, 275.53 μmol, 2 eq.) and 1-diazo-1-dimethoxyphosphoryl-propan-2-one (52.93 mg, 275.53 μmol, 2 eq.). The mixture was stirred at 25° C. for 12 h. Upon completion, the reaction was quenched by the addition of NH₄Cl (sat. aq., 10 mL). The mixture was extracted with EtOAc (3×20 mL). The combined organic layers were washed with brine (3×20 mL), dried over anhydrous Na₂SO₄, filtered and concentrated under reduced pressure to give a residue. The residue was purified by flash silica gel chromatography (ISCO®; 20 g SepaFlash® Silica Flash Column, Eluent of 0˜2.1% MeOH/DCM @ 30 mL/min) to afford 52-2 (41 mg, 72.07 μmol, 52.32% yield, 95% purity) as a colorless oil. LCMS: (ESI): m/z calcd. for C₂₇H₂₅Cl₂FN₅O₂ 540.1. [M+H]⁺, found 540.0.

A solution of 52-2 (40 mg, 74.02 μmol, 1 eq.) in dioxane (4 mL) and NH₃.H₂O (3.64 g, 25.97 mmol, 4 mL, 25% purity, 206.74 eq.) was stirred in a 30 mL sealed tube at 110° C. for 24 h. Upon completion, the reaction was quenched by the addition of NaHCO₃ (sat. aq., 5 mL). The mixture was extracted with EtOAc (3×20 mL). The combined organic layers were washed with brine (3×20 mL), dried over anhydrous Na₂SO₄, filtered and concentrated under reduced pressure to give a residue. The residue was purified by flash silica gel chromatography (ISCO®; 25 g SepaFlash® Silica Flash Column, Eluent of 0˜2.8% MeOH/DCM gradient @ 30 mL/min) to afford 52-3 (10 mg, 18.43 μmol, 24.90% yield, 96% purity) as a white solid. LCMS: (ESI): m/z calcd. for C₂₇H₂₇ClFN₆O₂, 521.2 [M+H]⁺, found 521.2.

A solution of 52-3 (10 mg, 18.50 μmol, 1 eq.) in HCl (4M aq., 1 mL) and THF (1 mL) was stirred at 25° C. for 12 h. Upon completion, the mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 21%-45%, 8 min) to afford 52 (5 mg, 10.40 μmol, 56.19% yield, 100% purity) as a white solid. LCMS: (ESI): m/z calcd. for C₂₄H₂₃ClFN₆O₂ 481.2. [M+H]⁺, found 481.2. ¹H NMR (400 MHz, CD₃OD) δ: 8.19 (s, 1H), 8.08 (s, 1H), 7.26 (s, 1H), 7.21 (d, J=3.8 Hz, 1H), 6.93 (d, J=10.0 Hz, 1H), 6.59 (d, J=3.5 Hz, 1H), 5.17-5.04 (m, 1H), 4.55 (t, J=6.8 Hz, 1H), 4.00 (d, J=6.8 Hz, 1H), 3.17-3.01 (m, 1H), 2.93 (dt, J=4.8, 12.7 Hz, 1H), 2.81 (s, 1H), 2.46 (dd, J=8.3, 12.8 Hz, 1H), 2.23-2.09 (m, 2H), 2.08-1.97 (m, 1H). ¹⁹F NMR (376 MHz, CD₃OD) δ: −125.74 (s, 1F).

### Example 48

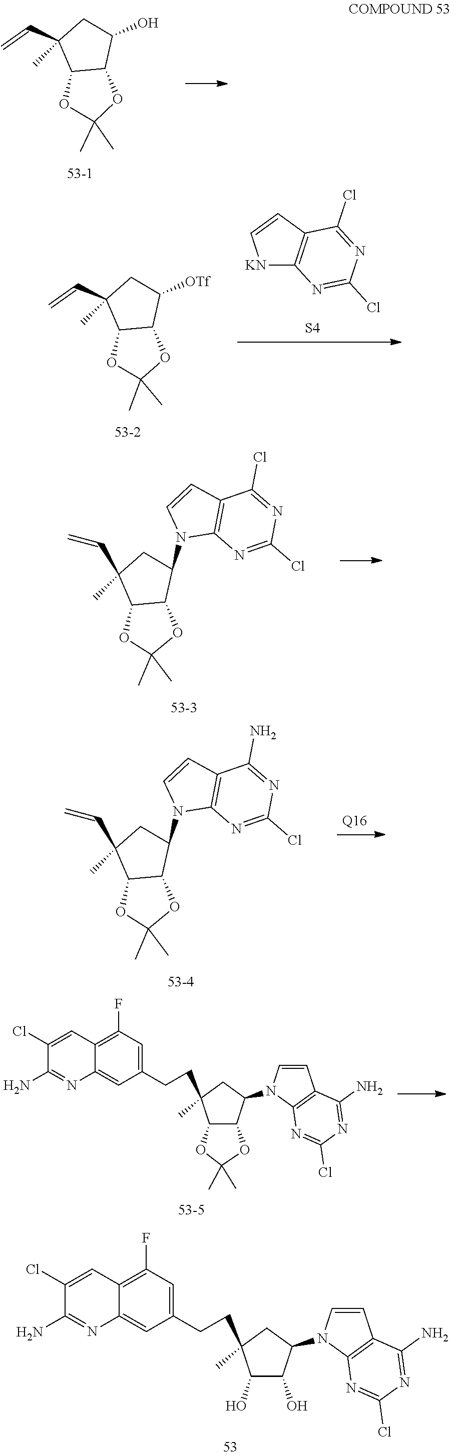

To a solution of 53-1 (1 g, 5.04 mmol, 1 eq.) in DCM (10 mL) were added Tf₂O (2.13 g, 7.57 mmol, 1.25 mL, 1.5 eq.) and pyridine (1.60 g, 20.18 mmol, 1.63 mL, 4 eq.). The mixture was stirred at 0° C. for 1 h. Upon completion, the reaction was quenched with H₂O (10 mL) at 0° C., then extracted with DCM (10 mL). The aqueous phase was extracted with DCM (3×20 mL). The combined organic layers were washed with brine (30 mL), dried over anhydrous Na₂SO₄, filtered and concentrated under reduced pressure to give 53-2 (1.8 g, crude) as a brown oil, which is used for the next step without further purification.

To a solution of 2,4-dichloro-7H-pyrrolo[2,3-d]pyrimidine (S4) (1.5 g, 7.98 mmol, 1.46 eq.) in DMF (5 mL) was added t-BuOK (832.57 mg, 7.42 mmol, 1.36 eq.). The mixture was stirred at 15° C. for 1 h. Compound 53-2 (1.8 g, 5.45 mmol, 1 eq.) was added at 0° C., and the mixture was stirred at 15° C. for 48 h under N₂. Upon completion, the reaction was partitioned between DCM (20 mL) and brine (20 mL). The aqueous phase was extracted with DCM (3×30 mL). The organic layers were combined, dried over Na₂SO₄, filtered and concentrated under reduced pressure to give a residue. The residue was purified by flash silica gel chromatography (ISCO®; 40 g SepaFlash® Silica Flash Column, Eluent of 0˜12% Ethyl acetate/Petroleum ether gradient @ 35 mL/min) to give 53-3 (0.51 g, 1.29 mmol, 25.6% yield, 93.173% purity) was obtained as colorless oil. LCMS: (ESI): m/z calcd. for C₁₇H₂₀Cl₂N₃O₂ 368.09 [M+H]⁺, found 369.9.

A solution of 53-3 (510 mg, 1.38 mmol, 1 eq.) in dioxane (10 mL) with sat. NH₃.H₂O (9.10 g, 64.91 mmol, 10 mL, 25% purity, 46.95 eq.) was stirred at 100° C. for 48 h in a 100 mL of sealed tube. Upon completion, the reaction was partitioned between EA (30 mL) and brine (30 mL). The organic phase was separated, and the aqueous phase was extracted with EA (3×30 mL). The organic layers were combined and dried over Na₂SO₄, filtered and concentrated under reduced pressure to give crude 53-4 (470 mg, 1.29 mmol, 93.5% yield, 95.419% purity) as a yellow oil, which was directly used for the next step without further purification. LCMS: (ESI): m/z calcd. for C₁₇H₂₂ClN₄O₂ 349.14 [M+H]⁺, found 349.1.

To the mixture of 53-4 (157.20 mg, 0.430 mmol, 1 eq.) in THF (4 mL) was added 9-BBN dimer (228.95 mg, 0.946 mmol, 2.2 eq.). The mixture was stirred at 50° C. for 1 h, and then cooled to 10° C. A solution of K₃PO₄ (456.39 mg, 2.15 mmol, 5 eq.) in H₂O (0.5 mL) was added, and the mixture was stirred at 10° C. for 0.2 h. Q16 (142.16 mg, 0.516 mmol, 1.2 eq.) and Pd(dppf)Cl₂ (31.46 mg, 0.043 mmol, 0.1 eq.) were added, and the mixture was stirred at 55° C. for 2 h under Ar. The mixture was partitioned between EA (20 mL) and water (10 mL). The aqueous phase was extracted with EA (3×20 mL). The organic layers were combined and washed with brine (30 mL), dried over anhydrous Na₂SO₄, filtered and concentrated under reduced pressure to give a residue. The residue was purified by column chromatography (SiO₂, Petroleum ether/Ethyl acetate (1:1) then DCM:MeOH (20:1)) to afford 53-5 (300 mg, 0.350 mmol, 77% yield, 63.729% purity) as a yellow solid. LCMS: (ESI): m/z calcd. for C₂₆H₂₈Cl₂FN₆O₂ 545.16 [M+H]⁺, found 545.1.

To a solution of 53-5 (300 mg, 0.350 mmol, 63.729% purity, 1 eq.) in THF (4 mL) was added HCl (4 M aq, 2 mL, 22.82 eq.). The mixture was stirred at 15° C. for 4 h, and then concentrated under reduced pressure to afford a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 34%-59%, 8 min) to give 53 (77 mg, 0.150 mmol, 42.88% yield, 98.64% purity) as an off-white solid. LCMS: (ESI): m/z calcd. for C₂₃H₂₄Cl₂FN₆O₂ 527.12 [M+Na]⁺, found 527.1. ¹H NMR (400 MHz, CD₃OD) δ: 8.19 (s, 1H), 7.24 (s, 1H), 7.18 (d, J=3.7 Hz, 1H), 6.94 (dd, J=1.1, 10.9 Hz, 1H), 6.56 (d, J=3.6 Hz, 1H), 4.97-4.91 (m, 1H), 4.58-4.48 (m, 1H), 3.93 (d, J=6.3 Hz, 1H), 2.92-2.71 (m, 2H), 2.09-1.96 (m, 2H), 1.93-1.79 (m, 2H), 1.23 (s, 3H). ¹⁹F NMR (400 MHz, CD₃OD) δ: −125.87.

### Example 49

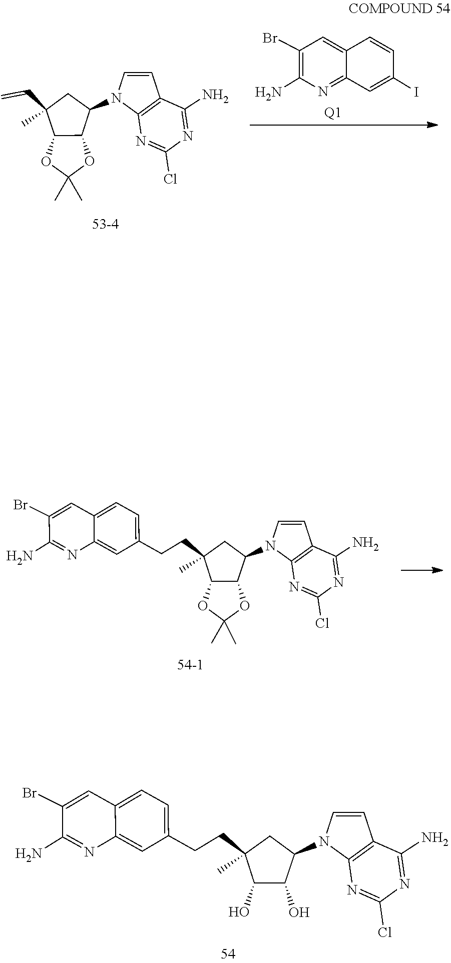

To a mixture of 53-4 (142 mg, 0.388 mmol, 1 eq.) in THF (4 mL) was added 9-BBN dimer (206.82 mg, 0.855 mmol, 2.2 eq.). The mixture was stirred at 50° C. for 1 h, and then cooled to 10° C. A solution of K₃PO₄ (412.26 mg, 1.94 mmol, 5 eq.) in H₂O (0.5 mL) was added, and the mixture was stirred at 10° C. for 0.2 h. Q1 (162.66 mg, 0.466 mmol, 1.2 eq.) and Pd(dppf)Cl₂ (28.42 mg, 0.0388 mmol, 0.1 eq.) were added, and the mixture was stirred at 55° C. for 2 h under Ar. The mixture was partitioned between EA (20 mL) and water (10 mL). The aqueous phase was extracted with EA (3×20 mL). The organic layers were combined, washed with brine (30 mL), dried over anhydrous Na₂SO₄, filtered and concentrated under reduced pressure to give a residue. The residue was purified by column chromatography (SiO₂, Petroleum ether/Ethyl acetate (2:1) then DCM:MeOH (20:1)) to afford 54-1 (270 mg, 0.307 mmol, 79% yield, 65% purity) as a yellow solid. LCMS: (ESI): m/z calcd. for C₂₆H₂₉BrClN₆O₂ 571.11 [M+H]⁺, found 573.0.

To a solution of 54-1 (270 mg, 0.307 mmol, 65% purity, 1 eq.) in THF (4 mL) was added HCl (4 M aq., 2 mL, 22.82 eq.), and the mixture was stirred at 15° C. for 4 h. The mixture was concentrated under reduced pressure to afford a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 32%-56%, 8 min) to give 54 (70 mg, 0.132 mmol, 43% yield, 100% purity) as a white solid. LCMS: (ESI): m/z calcd. for C₂₃H₂₅BrClN₆O₂ 531.08 [M+H]⁺, found 533.1. ¹H NMR (400 MHz, CD₃OD) δ: 8.26 (s, 1H), 7.55 (d, J=8.2 Hz, 1H), 7.40 (s, 1H), 7.20 (d, J=8.2 Hz, 1H), 7.17 (d, J=3.6 Hz, 1H), 6.56 (d, J=3.6 Hz, 1H), 4.98-4.91 (m, 1H), 4.52 (t, J=6.9 Hz, 1H), 3.93 (d, J=6.2 Hz, 1H), 2.93-2.70 (m, 2H), 2.11-1.93 (m, 2H), 1.92-1.79 (m, 2H), 1.23 (s, 3H).

### Example 50

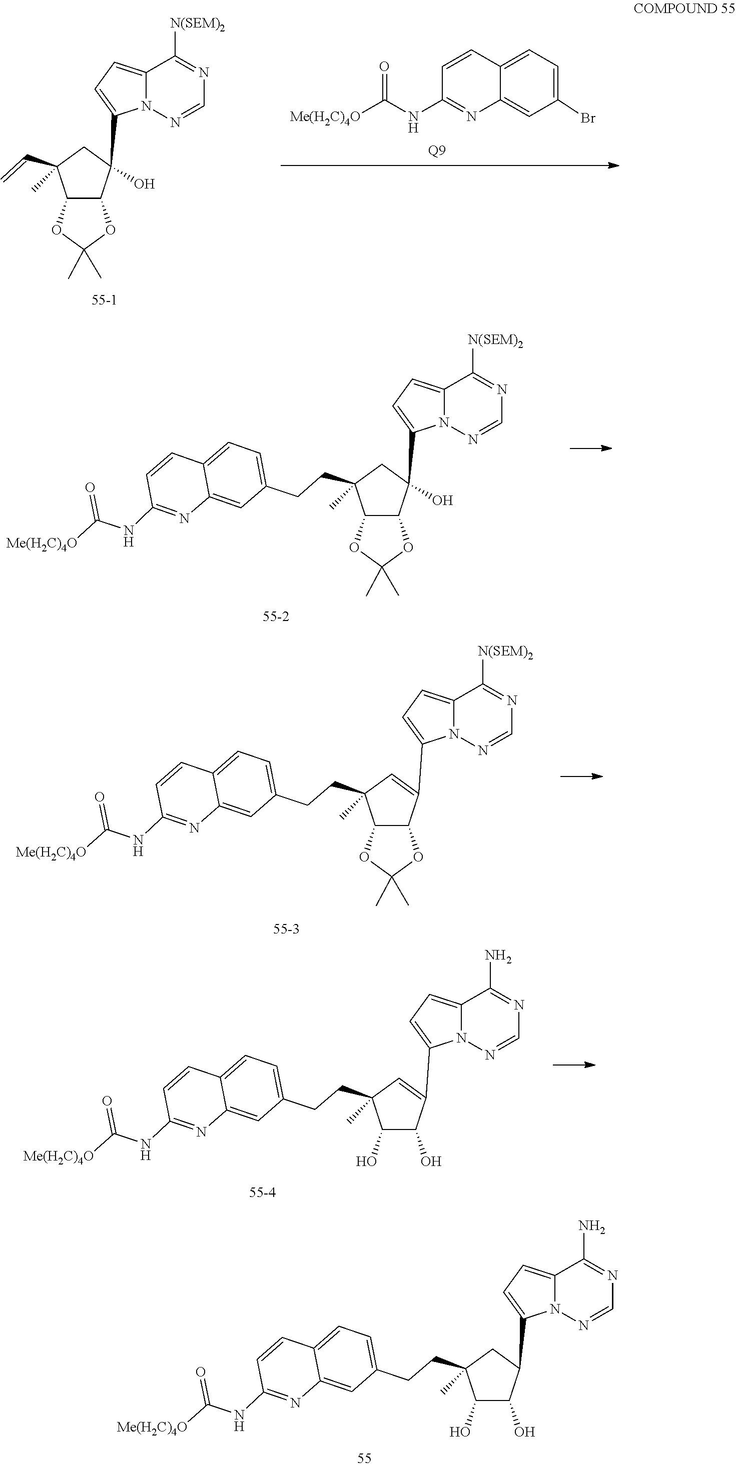

A mixture of 55-1 (613 mg, 1.04 mmol, 1 eq.) and 9-BBN dimer (627 mg, 2.59 mmol, 2.5 eq.) in THF (10 mL) was stirred at 50° C. for 2 h under Ar, and the cooled to rt. A solution of K₃PO₄ (1.10 g, 5.19 mmol, 5 eq.) in H₂O (1 mL) was added, and the mixture was stirred for 0.5 h. Q9 (419 mg, 1.24 mmol, 1.2 eq.) and Pd(dppf)Cl₂ (76 mg, 0.103 mmol, 0.1 eq.) were added, and the mixture was purged with Ar (3×) and then stirred at 70° C. for 12 h under Ar. The reaction was quenched by the addition of water (50 mL) and extracted with EA (3×100 mL). The combined organic layers were washed with brine (100 mL), dried over Na₂SO₄, filtered and concentrated under reduced pressure to give a residue. The residue was purified by column chromatography (SiO₂, Petroleum ether:Ethyl acetate=30:1 to 5:1) to afford 55-2 (550 mg, 608.80 μmol, 59% yield, 94% purity) as a white solid. LCMS: (ESI): m/z calcd. for C₄₄H₆₉N₆O₇Si₂ 849.47 [M+H]⁺, found 849.4.

To a solution of 55-2 (550 mg, 647.66 μmol, 1 eq.) in DCM (10 mL) was added DAST (1.04 g, 6.48 mmol, 855.70 μL, 10 eq.) at 0° C., and the mixture was stirred at 0° C. for 2 h. The reaction was quenched by addition of water (50 mL) and extracted with EA (3×100 mL). The combined organic layers were washed with brine (100 mL), dried over Na₂SO₄, filtered and concentrated under reduced pressure to give a residue. The residue was purified by column chromatography (SiO₂, Petroleum ether:Ethyl acetate=30:1 to 5:1) to afford 55-3 (450 mg, 498.08 μmol, 76.90% yield, 92% purity) as a white solid. LCMS: (ESI): m/z calcd. for C₄₄H₆₆N₆O₆Si₂Na 853.46 [M+Na]⁺, found 853.4.

To a solution of 55-3 (440 mg, 529.36 μmol, 1 eq.) in THF (8 mL) was added HCl (4 M, 4 mL), and the mixture was stirred at 25° C. for 5 h. Upon completion, the mixture was concentrated under reduced pressure to give a residue. The residue was dissolved in a mixed solvent of t-BuOH (8 mL) and H₂O (4 mL), and PPTS (664.15 mg, 2.64 mmol, 5 eq.) was added. The mixture was stirred at 50° C. for 3 h. The mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 17%-47%, 8 min) to afford 55-4 (200 mg, 373.15 μmol, 71% yield, 99% purity) as a white solid. LCMS: (ESI): m/z calcd. for C₂₉H₃₅N₆O₄ 531.26 [M+H]⁺, found 531.4.

To a solution of 55-4 (175 mg, 329.80 μmol, 1 eq.) in THF (30 mL) was added PtO₂ (875 mg, 3.85 mmol, 11.68 eq.), and the mixture was stirred under H₂ (15 psi) atmosphere at 25° C. for 18 h. The mixture was filtered through a pad of Celite to remove PtO₂. The filtrate was concentrated under reduced pressure to afford a residue. The residue was purified by SFC (column: Chiralcel OJ-3 100; Á4.6 mm I.D., 3 um; Mobile phase: A: CO₂B: ethanol (0.05% DEA); Isocratic: 40% B; Flow rate: 2.8 mL/min; Column temp.: 35° C.; ABPR: 1500 psi; to afford 55 (70 mg, 128.79 μmol, 39% yield, 98% purity) as a white solid. LCMS: (ESI): m/z calcd. for C₂₉H₃₇N₆O₄ 533.28 [M+H]⁺, found 533.4. ¹H NMR (400 MHz, DMSO) δ: 10.33 (s, 1H), 8.24 (d, J=9.0 Hz, 1H), 7.99 (d, J=8.8 Hz, 1H), 7.81-7.75 (m, 2H), 7.53 (s, 3H), 7.32 (br d, J=8.2 Hz, 1H), 6.81 (d, J=4.4 Hz, 1H), 6.51 (d, J=4.2 Hz, 1H), 4.69 (br d, J=5.7 Hz, 1H), 4.41 (br d, J=5.3 Hz, 1H), 4.29 (br d, J=6.8 Hz, 1H), 4.11 (t, J=6.6 Hz, 2H), 3.72 (br t, J=5.5 Hz, 1H), 3.61 (br d, J=9.3 Hz, 1H), 2.80 (br dd, J=4.4, 12.3 Hz, 1H), 2.75-2.67 (m, 1H), 1.93 (br dd, J=8.7, 12.7 Hz, 1H), 1.80-1.71 (m, 1H), 1.68-1.56 (m, 3H), 1.50 (br t, J=11.8 Hz, 1H), 1.38-1.31 (m, 4H), 1.12 (s, 3H), 0.92-0.87 (m, 3H).

### Example 51

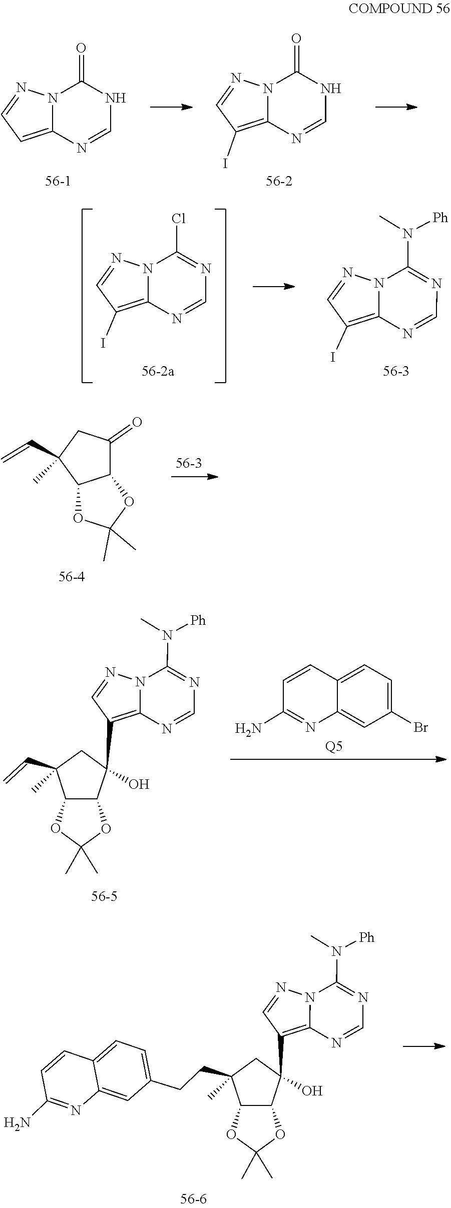

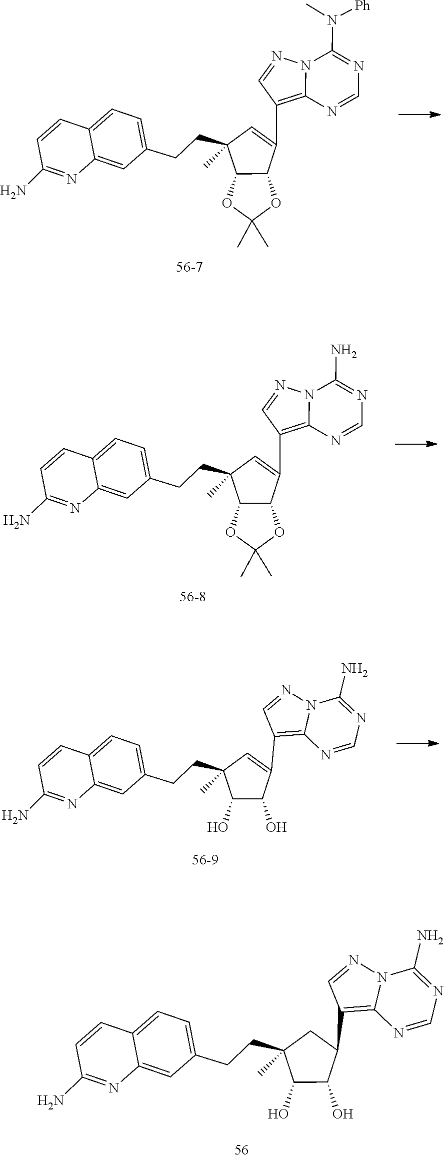 [SMILES: CN(c1ccccc1)c1ncnc2c(C3=C[C@](C)(CCc4ccc5ccc(N)nc5c4)[C@H]4OC(C)(C)O[C@@H]34)cnn12 ; CC1(C)O[C@H]2C(c3cnn4c(N)ncnc34)=C[C@](C)(CCc3ccc4ccc(N)nc4c3)[C@H]2O1 ; C[C@]1(CCc2ccc3ccc(N)nc3c2)C=C(c2cnn3c(N)ncnc23)[C@H](O)[C@@H]1O ; C[C@]1(CCc2ccc3ccc(N)nc3c2)C[C@@H](c2cnn3c(N)ncnc23)[C@H](O)[C@@H]1O]

To a solution of 56-1 (5 g, 36.73 mmol, 1 eq.) in DMF (50 mL) was added NIS (10.74 g, 47.76 mmol, 1.3 eq.), and the mixture was stirred at 40° C. for 3.5 h. The mixture was poured into water (200 mL) and filtered to give a residue. The residue was triturated with water (50 mL×2) to give 56-2 (6.49 g, 23.78 mmol, 64% yield, 96% purity) as a brown solid. LCMS: (ESI): m/z calcd. for C₅H₄IN₄O 262.94 [M+H]⁺, found 262.9.

A solution of 56-2 (6.49 g, 24.77 mmol, 1 eq.), POCl₃ (83.73 g, 546.07 mmol, 50.75 mL, 22.05 eq.), and DMAP (9.08 g, 74.31 mmol, 3 eq.) was stirred at 105° C. for 2 h. The mixture was concentrated under reduced pressure to give 56-2a (15 g, crude) as a brown solid. To a solution of 56-2a (7.5 g, 26.74 mmol, 1 eq.) in DCM (80 mL) was added N-methylaniline (11.46 g, 106.97 mmol, 11.61 mL, 4 eq.) dropwise at 0° C., followed by addition of TEA (16.24 g, 160.45 mmol, 22.33 mL, 6 eq.). The mixture was stirred at 25° C. for 12 h. The mixture was diluted with H₂O (50 mL) and extracted with DCM (2×50 mL). The combined organic layers were washed with brine (50 mL), dried over anhydrous Na₂SO₄, filtered and concentrated under reduced pressure to give a residue. The residue was purified by flash silica gel chromatography (ISCO®; 120 g SepaFlash® Silica Flash Column, Eluent of 0˜20% Ethyl acetate/Petroleum ether gradient @ 50 mL/min) to give 56-3 (6.68 g, 18.83 mmol, 76% yield, 99% purity) as a white solid (Note: two parallel reactions from 56-1a were carried out, then combined for purification). LCMS: (ESI): m/z calcd. for C₁₂H₁₁IN₅ 352.0 [M+H]⁺, found 351.9.

To a solution of 56-3 (5.37 g, 15.29 mmol, 3 eq.) in THF (15 mL) was added i-PrMgCl.LiCl (1.3 M, 12.94 mL, 3.3 eq.) dropwise. The mixture was stirred at −20° C. for 10 min, warmed to 0° C. and then stirred at 0° C. for 1 h. A solution of 56-4 (1 g, 5.10 mmol, 1 eq.) in THF (10 mL) was added dropwise to the mixture at −20° C., and then stirred at 0° C. for 1 h 20 min. The reaction was quenched with NH₄Cl solution (30 mL) and extracted with EA (2×30 mL). The combined organic layers were washed with brine (50 mL), dried over anhydrous Na₂SO₄, filtered and concentrated under reduced pressure to give a residue. The residue was purified by flash C-18 column chromatography (40 g, 0%˜60% MeCN, 0.5 mL NH₃.H₂O in 1 L H₂O, 30 mL/min) to give 56-5 (1.01 g, 2.30 mmol, 45% yield) as a white solid. LCMS: (ESI): m/z calcd. for C₂₃H₂₈N₅O₃ 422.21 [M+H]⁺, found 422.1.

To a solution of 56-5 (1.01 g, 2.40 mmol, 1 eq.) in THF (25 mL) was added 9-BBN dimer (1.45 g, 5.99 mmol, 2.5 eq.). The mixture was stirred at 50° C. for 2 h under N₂ and then cooled to rt. A solution of K₃PO₄ (2.54 g, 11.98 mmol, 5 eq.) in H₂O (2.5 mL) was added, and the mixture was stirred at rt for 0.5 h. 7-bromoquinolin-2-amine Q5 (694.89 mg, 3.12 mmol, 1.3 eq.) and Pd(dppf)Cl₂ (175.34 mg, 0.239 mmol, 0.1 eq.) were added, and the mixture was stirred at 60° C. for 12 h. The mixture was diluted with H₂O (20 mL) and extracted with EA (2×20 mL). The combined organic layers were washed with brine (50 mL), dried over anhydrous Na₂SO₄, filtered and concentrated under reduced pressure to give a residue. The residue was purified by column chromatography (SiO₂, Petroleum ether:Ethyl acetate=2:1 to 1:1 to DCM:MeOH=100:1 to 20:1) to give 56-6 (1 g, 1.71 mmol, 71% yield) as a yellow solid. LCMS: (ESI): m/z calcd. for C₃₂H₃₆N₇O₃ 566.28 [M+H]⁺, found 566.2.

To a solution of 56-6 (0.95 g, 1.68 mmol, 1 eq.) in DCM (15 mL) was added DAST (1.35 g, 8.40 mmol, 1.11 mL, 5 eq.) at 0° C., and the mixture was stirred at 0° C. for 0.5 h. reaction was quenched by addition of NaHCO₃ solution (10 mL), diluted with DCM (5 mL) and extracted with DCM (2×10 mL). The combined organic layers were washed with brine (20 mL), dried over anhydrous Na₂SO₄, filtered and concentrated under reduced pressure to give a residue. The residue was purified by column chromatography (SiO₂, DCM:MeOH=100:1 to 20:1) to give 56-7 (708 mg, 1.27 mmol, 75% yield, 98% purity) as a yellow solid. LCMS: (ESI): m/z calcd. for C₃₂H₃₄N₇O₂ 548.27 [M+H]⁺, found 548.1.

To a solution of 56-7 (708 mg, 1.29 mmol, 1 eq.) in dioxane (15 mL) was added NH₃.H₂O (13.65 g, 97.37 mmol, 15 mL, 25% purity), and the mixture was stirred at 100° C. for 24 h. The mixture was concentrated under reduced pressure to give a residue. The residue was purified by column chromatography (SiO₂, DCM:MeOH=50:1 to 12:1) to give 56-8 (238 mg, 483.78 μmol, 37% yield, 93% purity) as a white solid. LCMS: (ESI): m/z calcd. for C₂₅H₂₈N₇O₂ 458.22 [M+H]⁺, found 458.3.

To a solution of 56-8 (228 mg, 498.33 μmol, 1 eq.) in THF (4 mL) was added HCl (4 M aq., 2.00 mL), and the mixture was stirred at 25° C. for 12 h. The mixture was concentrated under reduced pressure to give a residue. The residue was purified by prep-HPLC (column: Phenomenex Gemini-NX 80*40 mm*3 um; mobile phase: [water (0.05% NH₃H₂O+10 mM NH₄HCO₃)-ACN]; B %: 40%-60%, 8 min) to give 56-9 (160 mg, 375.60 μmol, 75% yield, 98% purity) as a white solid. LCMS: (ESI): m/z calcd. for C₂₂H₂₄N₇O₂ 418.19 [M+H]⁺, found 418.1.

To a solution of 56-9 (30 mg, 71.86 μmol, 1 eq.) in a mixed solvent of THF (7.5 mL) and MeOH (1.5 mL) was added PtO₂ (150 mg, 660.57 μmol, 9.19 eq.). The suspension was degassed/purged with H₂ (3×) and stirred under H₂ (15 Psi) atmosphere at 25° C. for 4 h. The mixture was filtered through a pad of Celite to remove PtO₂. The filtrate was concentrated under reduced pressure to give a white solid (30 mg, crude product). The crude product was combined with another batch (25 mg scale) for prep-HPLC (column: Phenomenex Gemini-NX 80*40 mm*3 um; mobile phase: [water (0.05% NH₃H₂O+10 mM NH₄HCO₃)-ACN]; B %: 12%-42%, 8 min) to give 56 (impure, 10 mg, 23.39 μmol, 98.1% purity by HPLC, NMR shows an impurity) as a white solid, which was confirmed by LCMS, HPLC and ¹H NMR. LCMS: (ESI): m/z calcd. for C₂₂H₂₆N₇O₂ 420.2 [M+H]⁺, found 420.3. ¹H NMR (400 MHz, CD₃OD) δ: 8.28 (d, J=9.3 Hz, 1H), 8.01 (d, J=9.8 Hz, 2H), 7.79 (d, J=8.3 Hz, 1H), 7.50 (s, 1H), 7.42 (d, J=8.5 Hz, 1H), 6.99 (d, J=9.3 Hz, 1H), 4.30 (dd, J=6.3, 8.0 Hz, 1H), 3.86 (d, J=6.0 Hz, 1H), 3.44-3.36 (m, 1H), 2.97-2.80 (m, 2H), 2.03-1.94 (m, 1H), 1.89-1.74 (m, 3H), 1.22 (s, 3H).

Impure 56 was further purified by SFC (column: DAICEL CHIRALPAK AD (250 mm*30 mm, 10 um); mobile phase: [0.1% NH₃H₂O ETOH]; B %: 60%-60%, min) to give 56 (2 mg, 4.57 μmol, 19% yield, 95.9% purity) as a white solid, which was confirmed by LCMS, HPLC and ¹H NMR. LCMS: (ESI): m/z calcd. for C₂₂H₂₆N₇O₂ 420.2 [M+H]⁺, found 420.3. ¹H NMR (400 MHz, CD₃OD) δ: 8.01 (s, s, 2H), 7.93 (d, J=9.0 Hz, 1H), 7.58 (d, J=8.2 Hz, 1H), 7.37 (s, 1H), 7.16 (dd, J=1.5, 8.1 Hz, 1H), 6.78 (d, J=8.9 Hz, 1H), 4.30 (dd, J=6.1, 8.2 Hz, 1H), 3.85 (d, J=6.1 Hz, 1H), 3.40 (td, J=8.4, 11.1 Hz, 1H), 2.90-2.71 (m, 2H), 1.99 (m, 1H), 1.87-1.72 (m, 3H), 1.21 (s, 3H).

### Example 52

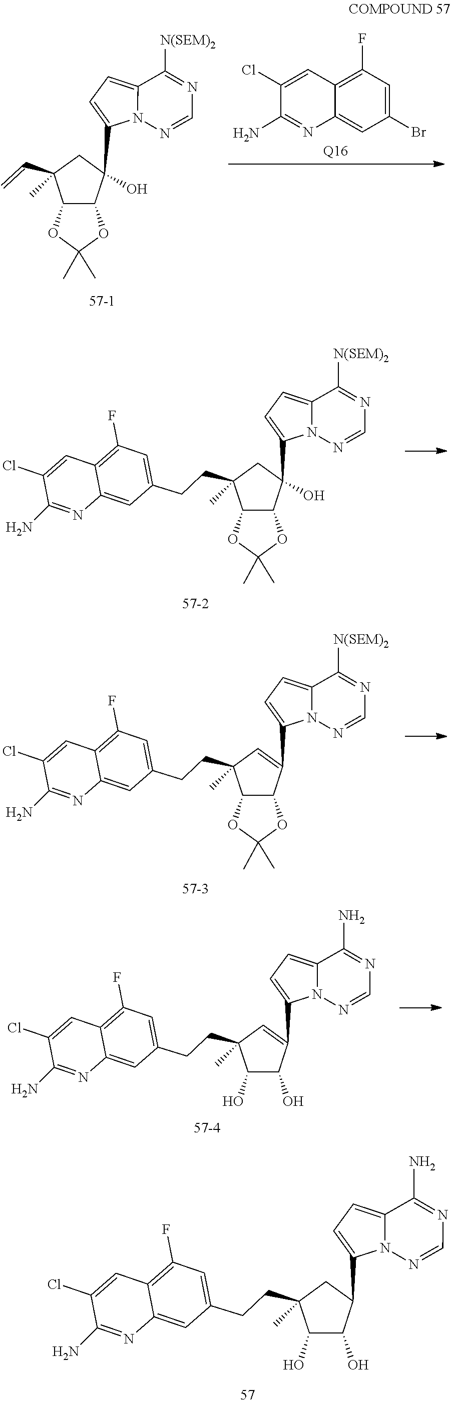

To a mixture of 57-1 (500 mg, 846.17 μmol, 1 eq.) in THF (10 mL) was added 9-BBN dimer (512 mg, 2.12 mmol, 2.5 eq.). The mixture was stirred at 50° C. for 2 h under N₂, and then cooled to 25° C. A solution of K₃PO₄ (898 mg, 4.23 mmol, 5 eq.) in H₂O (1 mL) was added, and the mixture was stirred for 0.5 h. Q16 (280 mg, 1.02 mmol, 1.2 eq.) and Pd(dppf)Cl₂ (62 mg, 0.084 mmol, 0.1 eq.) were added, and the mixture was purged with N₂ (3×). The mixture was stirred at 60° C. for 12 h. The mixture was diluted with water (5 mL) and extracted with EA (2×5 mL). The combined organic layers were washed with brine (10 mL), dried over Na₂SO₄, filtered and concentrated under reduced pressure to give a residue. The residue was purified by flash silica gel chromatography (ISCO®; 40 g SepaFlash® Silica Flash Column, Eluent of 5-20% Ethyl acetate/Petroleum ether gradient @ 30 mL/min) to afford 57-2 (415 mg, 519.60 μmol, 61% yield, 98.6% purity) as a yellow solid. LCMS: (ESI): m/z calcd. for C₃₈H₅₇ClFN₆O₅Si₂ 787.35 [M+H]⁺, found 787.1.

To a solution of 57-2 (415 mg, 526.98 μmol, 1 eq.) in DCM (7 mL) was added DAST (424.72 mg, 2.63 mmol, 348.13 μL, 5 eq.) at 0° C. The mixture was stirred at 0° C. for 1 h. The mixture was diluted with water (10 mL) and extracted with DCM (2×5 mL). The combined organic layers were washed with brine (10 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜20% Ethyl acetate/Petroleum ether gradient @ 30 mL/min) to afford 57-3 (274 mg, 341.84 μmol, 64% yield, 96% purity) as a white solid. LCMS: (ESI): m/z calcd. for C₃₈H₅₄ClFN₆O₄Si₂Na 791.34 [M+Na]⁺, found 791.2.

To a solution of 57-3 (207 mg, 0.269 mmol, 1 eq.) in THF (4 mL) was added HCl (aq., 4 M, 2 mL). The mixture was stirred at 25° C. for 6 h. The mixture was concentrated under reduced pressure to give a residue. The residue was dissolved in t-BuOH (10 mL) and H₂O (5 mL), and PPTS (352.57 mg, 1.40 mmol, 5 eq.) was added. The mixture was stirred at 50° C. for 12 h. The mixture was concentrated under reduced pressure to give a residue. The residue was purified by reverse-phase flash column (40 g C-18 column, 0%˜60% MeCN in water (0.5 mL NH₃.H₂O in 1 L H₂O)@ 35 mL/min) to afford 57-4 (85 mg, 0.171 mmol, 63% yield, 94.8% purity) as a white solid. LCMS: (ESI): m/z calcd. for C₂₃H₂₃ClFN₆O₂ 469.15 [M+H]⁺, found 469.1.

To a solution of 57-4 (30 mg, 0.063 mmol, 1 eq.) in MeOH (12 mL) was added Crabtree's catalyst (180.00 mg, 223.63 μmol, 3.5 eq.). The suspension was degassed and purged with H₂ (3×). The mixture was stirred under H₂ (15 Psi) at 25° C. for 5 h. mixture was treated with TMT (1,3,5-triazine-2,4,6-trithiol) and then kept overnight. The resulting suspension was filtered and concentrated under reduced pressure to give a residue. The residue was purified by prep-TLC (SiO₂, DCM:MeOH=7:1) to give a syrup (10 mg), which was further purified by SFC (column: DAICEL CHIRALPAK IG (250 mm*30 mm, 10 um); mobile phase: [0.1% NH₃H₂O IPA]; B %: 60%-60%, min) to give impure 57 (8 mg, 16.87 μmol, 26% yield, 99.29% purity, contains some grease) as a yellow solid. It was combined with another batch (7 mg of impure product) and triturated with EA (2×0.5 mL) and isopropyl ether (0.5 mL) to afford 57 (10 mg, 21.06 μmol, 99.18% purity) as a white solid. LCMS: (ESI): m/z calcd. for C₂₃H₂₅ClFN₆O₂ 471.16 [M+H]⁺, found 471.2. ¹H NMR (400 MHz, CD₃OD) δ: 8.17 (s, 1H), 7.76 (s, 1H), 7.21 (s, 1H), 6.87 (dd, J=1.3, 10.9 Hz, 1H), 6.84 (d, J=4.4 Hz, 1H), 6.54 (d, J=4.4 Hz, 1H), 4.44 (dd, J=6.3, 7.2 Hz, 1H), 3.85 (d, J=6.1 Hz, 1H), 3.78-3.67 (m, 1H), 2.87-2.67 (m, 2H), 2.03 (dd, J=8.9, 12.9 Hz, 1H), 1.87-1.68 (m, 3H), 1.21 (s, 3H).

### Example 53

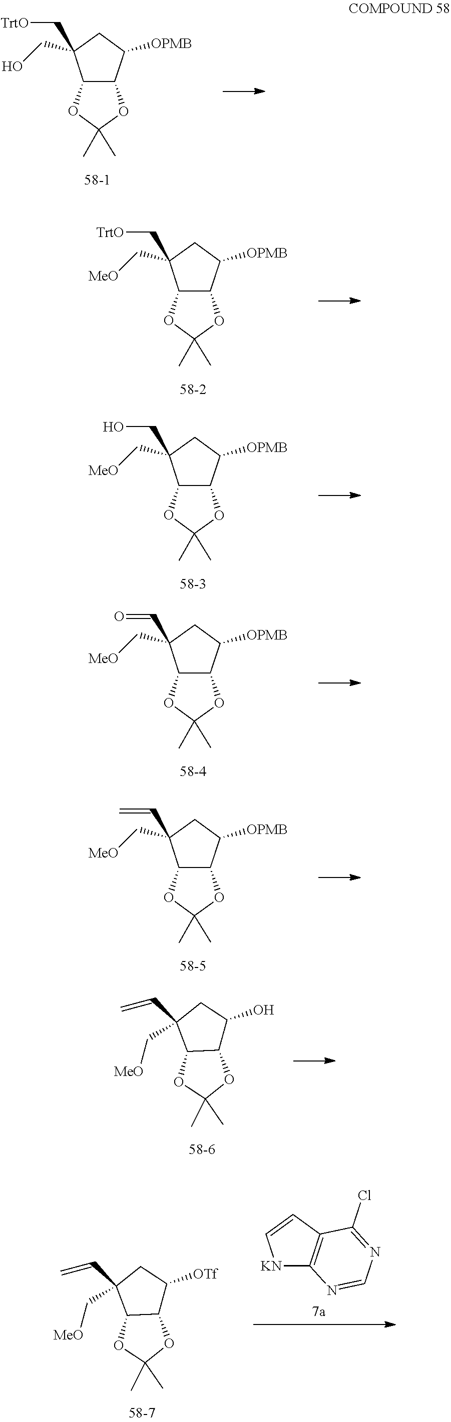

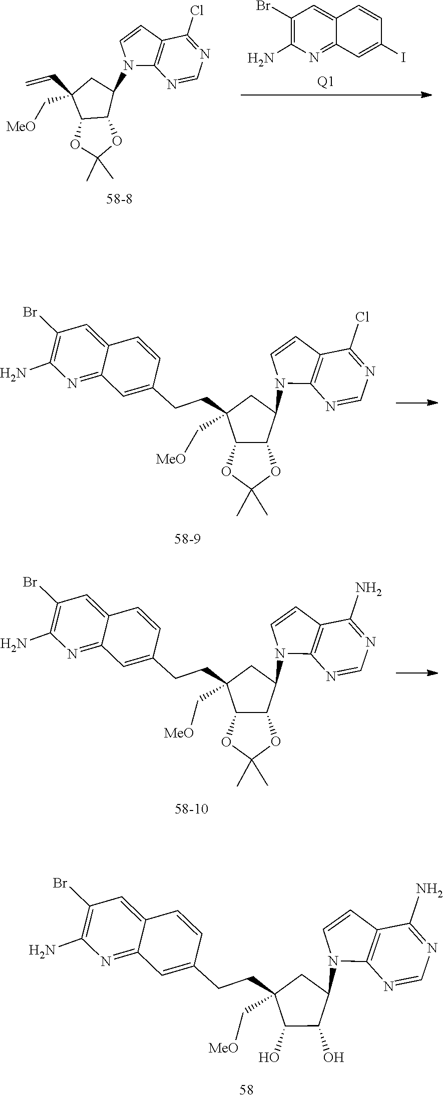

To a solution of 58-1 (2 g, 3.44 mmol, 1 eq.) in THF (20 mL) was added NaH (826.50 mg, 20.66 mmol, 60% purity, 6 eq.) in portions at 0° C. under N₂. The mixture was stirred at 0° C. for 0.5 h. Mel (8.18 g, 57.63 mmol, 3.59 mL, 16.73 eq.) was added dropwise at 0° C. The mixture was stirred at 15° C. for 12 h under N₂. The reaction was quenched by dropwise addition of NH₄Cl (sat., aq., 50 mL) at 0° C. and extracted with EA (3×30 mL). The combined organic layer was washed with brine (30 mL), dried over Na₂SO₄, filtered and concentrated in vacuum. The residue was purified by a silica gel column (PE:EA=100:0 to 5:1) to give 58-2 (1.1 g, 1.63 mmol, 47% yield, 88% purity) as a colorless oil. LCMS: (ESI): m/z calcd. for C₃₈H₄₂O₆Na, 617.30 [M+Na]+, found 617.1.

To a solution of 58-2 (0.8 g, 1.18 mmol, 88% purity, 1 eq.) and Et₃SiH (1.38 g, 11.84 mmol, 1.89 mL, 10 eq.) in DCM (20 mL) was added TFA (539.89 mg, 4.73 mmol, 350.58 μL, 4 eq.) at 0° C. The mixture was stirred at 0° C. for 0.5 h. The reaction was quenched with sat. NaHCO₃ solution (30 mL) and extracted with EA (3×30 mL). The combined organic layers were washed with brine (90 mL), dried over anhydrous Na₂SO₄, filtered and concentrated under reduced pressure to give a residue. The residue was purified by flash silica gel chromatography (ISCO®; 12 g CombiFlash® Silica Flash Column, Eluent of 0˜50% Ethyl acetate/Petroleum ether gradient @ 35 mL/min) to afford 58-3 (0.31 g, 0.859 mmol, 97% purity, 74% yield) as a colorless syrup. LCMS: (ESI): m/z calcd. for C₁₉H₂₈O₆Na 375.2, [M+Na]⁺, found 375.2.

To a solution of 58-3 (0.31 g, 0.859 mmol, 1 eq.) in CH₃CN (60 mL) was added IBX (738.93 mg, 2.64 mmol, 3 eq.). The mixture was stirred at 60° C. for 3 h. The mixture was cooled to 10° C. and diluted with EA (100 mL). The precipitation was filtered to move insoluble matters, and the filtrate was concentrated to afford 58-4 (0.34 g, crude) as a yellow gum. LCMS: (ESI): m/z calcd. for C₁₉H₃₀O₆N 368.2, [M+NH₄]⁺, found 368.3.

To a solution of MePPh₃Br (980.18 mg, 2.74 mmol, 5 eq.) in THF (20 mL) was added t-BuOK (1 M in THF, 2.20 mL, 4 eq.), and the mixture was stirred at 10° C. for 0.5 h. A solution of 58-4 (0.34 g, 0.549 mmol, 56.557% purity, 1 eq.) in THF (10 mL) was added. The mixture was stirred at 10° C. for 1 h. The reaction was quenched with sat. NH₄Cl (30 mL). The mixture was extracted with EA (3×30 mL) and washed with brine (60 mL). The separated organic layer was dried over anhydrous Na₂SO₄, filtered and concentrated to afford a residue. The residue was purified by flash silica gel chromatography (ISCO®; 25 g CombiFlash® Silica Flash Column, Eluent of 0˜20% Ethyl acetate/Petroleum ether gradient @ 35 mL/min) to afford 58-5 (210 mg, 0.557 mmol, 92.4% purity, 63% yield over 2 steps) as a colorless gum. LCMS: (ESI): m/z calcd. for C₂₀H₃₂O₅N 366.2, [M+NH₄]⁺ found 366.3.

To a solution of 58-5 (160 mg, 0.424 mmol, 92.4% purity, 1 eq.) in a mixed solvent of DCM (6 mL) and H₂O (0.6 mL) was added DDQ (192.64 mg, 0.849 mmol, 2 eq.). The mixture was stirred at 10° C. for 18 h. The reaction was quenched with sat. NaHCO₃ solution (20 mL), and then extracted with EA (3×20 mL). The combined organic layers were washed with brine (60 mL), dried over anhydrous Na₂SO₄, filtered and concentrated under reduced pressure to give a crude. The residue was purified by flash silica gel chromatography (ISCO®; 25 g CombiFlash® Silica Flash Column, Eluent of 0˜25% Ethyl acetate/Petroleum ether gradient @ 35 mL/min) to afford 58-6 (90 mg, 0.394 mmol, 93% yield) as alight yellow gum. ¹H NMR (400 MHz, CDCl₃) δ: 5.83 (dd, J=11.0, 17.8 Hz, 1H), 5.20 (d, J=11.0 Hz, 1H), 5.09 (d, J=18.1 Hz, 1H), 4.54 (dd, J=0.9, 5.4 Hz, 1H), 4.50-4.44 (m, 1H), 4.05 (ddd, J=4.4, 6.0, 10.4 Hz, 1H), 3.58 (d, J=8.8 Hz, 1H), 3.34 (s, 3H), 3.29 (d, J=8.8 Hz, 1H), 2.42 (d, J=10.0 Hz, 1H), 1.99 (dd, J=6.4, 12.2 Hz, 1H), 1.52 (s, 3H), 1.47 (d, J=12.0 Hz, 1H), 1.38 (s, 3H).

To a solution of 58-6 (100 mg, 0.438 mmol, 1 eq.) and pyridine (138.6 mg, 1.75 mmol, 0.141 mL, 4 eq.) in DCM (5 mL) was added Tf₂O (185.3 mg, 0.657 mmol, 0.108 mL, 1.5 eq.) dropwise at 0° C. The mixture was stirred at 0° C. for 2 h. The reaction was quenched with H₂O (10 mL), and then extracted with DCM (3×20 mL). The combined organic layers were washed with brine (60 mL), dried over anhydrous Na₂SO₄, filtered and concentrated under reduced pressure to afford 58-7 (160 mg, crude, 0.444 mmol) as a brown oil. ¹H NMR (400 MHz, CDCl₃) δ: 5.84 (dd, J=11.2, 17.9 Hz, 1H), 5.27 (d, J=11.0 Hz, 1H), 5.13 (d, =17.8 Hz, 1H), 4.96 (td, J=6.3, 10.8 Hz, 1H), 4.64 (t, J=5.4 Hz, 1H), 4.53 (d, J=5.0 Hz, 1H), 3.59 (d, J=8.8 Hz, 1H), 3.38-3.36 (m, 1H), 3.35 (s, 3H), 2.21 (dd, J=6.8, 12.3 Hz, 1H), 2.13-1.99 (m, 1H), 1.54 (s, 3H), 1.37 (s, 3H).

To a solution of 7a (127.7 mg, 666 μmol, 1.5 eq.) in DMF (2 mL) was added a solution of 58-7 (160 mg, 0.444 mmol, 1 eq.) in DMF (2 mL). The mixture was stirred at 10° C. for 48 h. The mixture was added H₂O (10 mL), and then extracted with EA (3×10 mL). The combined organic layers were washed with brine (30 mL), dried over anhydrous Na₂SO₄, filtered and concentrated under reduced pressure to give a residue. The residue was purified by flash silica gel chromatography (ISCO®; 12 g CombiFlash® Silica Flash Column, Eluent of 0˜10% Ethyl acetate/Petroleum ether gradient @ 30 mL/min) to afford 58-8 (90 mg, 0.242 mmol, 97.9% purity, 55% yield over 2 steps) as a colorless gum. LCMS: (ESI): m/z calcd. for C₁₈H₂₃O₃N₃Cl 364.1, [M+H]⁺, found 364.2.

To a solution of 58-8 (46 mg, 0.124 mmol, 97.953% purity, 1 eq.) in THF (3 mL) was added 9-BBN dimer (65.94 mg, 0.272 mmol, 2.2 eq.). The mixture was stirred at 50° C. for 1.5 h, and then cooled to 10° C. A solution of K₃PO₄ (131.44 mg, 0.619 mmol, 5 eq.) in H₂O (0.3 mL) was added, and the mixture was stirred at 10° C. for 0.5 h. Q1 (51.86 mg, 0.149 mmol, 1.2 eq.) and Pd(dppf)Cl₂ (9.06 mg, 0.012 mmol, 0.1 eq.) were added. The mixture was degassed with N₂ (3×) and stirred at 50° C. for 2 h. The mixture was diluted with brine (10 mL), and then extracted with EA (3×10 mL). The combined organic layers were washed with brine (30 mL), dried over anhydrous Na₂SO₄, filtered and concentrated under reduced pressure to give a residue. The residue was purified by flash silica gel chromatography (ISCO®; 12 g CombiFlash® Silica Flash Column, Eluent of 0˜50% Ethyl acetate/Petroleum ether gradient @ 30 mL/min) to afford 58-9 (36 mg, 0.058 mmol, 47% yield, 94.2% purity) as a yellow gum. LCMS: (ESI): m/z calcd. for C₂₇H₃₀BrClN₅O₃ 586.1, [M+2+H]⁺, found 588.1.

To a solution of 58-9 (36 mg, 0.058 mmol, 94% purity, 1 eq.) in dioxane (5 mL) was added NH₃.H₂O (4.55 g, 32.46 mmol, 5 mL, 25% purity, 561.78 eq.). The mixture was stirred at 100° C. for 48 h in a 30 mL sealed tube. The mixture was treated with brine (10 mL) and extracted with EA (3×20 mL). The combined organic layers were washed with brine (50 mL), dried over anhydrous Na₂SO₄, filtered and concentrated under reduced pressure to afford 58-10 (34 mg, 0.050 mmol, 87% yield, 84% purity) as a brown syrup. LCMS: (ESI): m/z calcd. for C₂₇H₃₂O₃N₆Br 567.2, [M+H]⁺, found 567.2.

To a solution of 58-10 (34 mg, 0.050 mmol, 83.799% purity, 1 eq.) in THF (4 mL) was added HCl (4 M aq., 2 mL, 159.34 eq.). The mixture was stirred at 10° C. for 3 h. The mixture was concentrated to give the residue. The residue was purified by prep-HPLC (basic condition; column: Phenomenex Gemini-NX 80*40 mm*3 um; mobile phase: [water (0.05% NH₃.H₂O+10 mM NH₄HCO₃)-ACN]; B %: 23%-49%, 8 min) to afford 58 (17 mg, 0.032 mmol, 64% yield, 99.1% purity) as a white solid. LCMS: (ESI): m/z calcd. for C₂₄H₂₈O₃N₆Br 527.1, [M+2+H]⁺, found 529.2. ¹H NMR (400 MHz, CD₃OD) δ: 8.27 (s, 1H), 8.08 (s, 1H), 7.57 (d, J=8.3 Hz, 1H), 7.43 (s, 1H), 7.23 (d, J=3.8 Hz, 1H), 7.20 (d, J=8.3 Hz, 1H), 6.59 (d, J=3.5 Hz, 1H), 5.00 (q, J=9.5 Hz, 1H), 4.62 (dd, J=5.4, 8.9 Hz, 1H), 3.99 (d, J=5.3 Hz, 1H), 3.70 (d, J=9.0 Hz, 1H), 3.51 (d, J=9.3 Hz, 1H), 3.43 (s, 3H), 2.92-2.70 (m, 2H), 2.17 (dd, J=9.3, 13.6 Hz, 1H), 1.99 (br t, J=8.4 Hz, 2H), 1.88 (dd, J=10.0, 13.6 Hz, 1H).

### Example 54

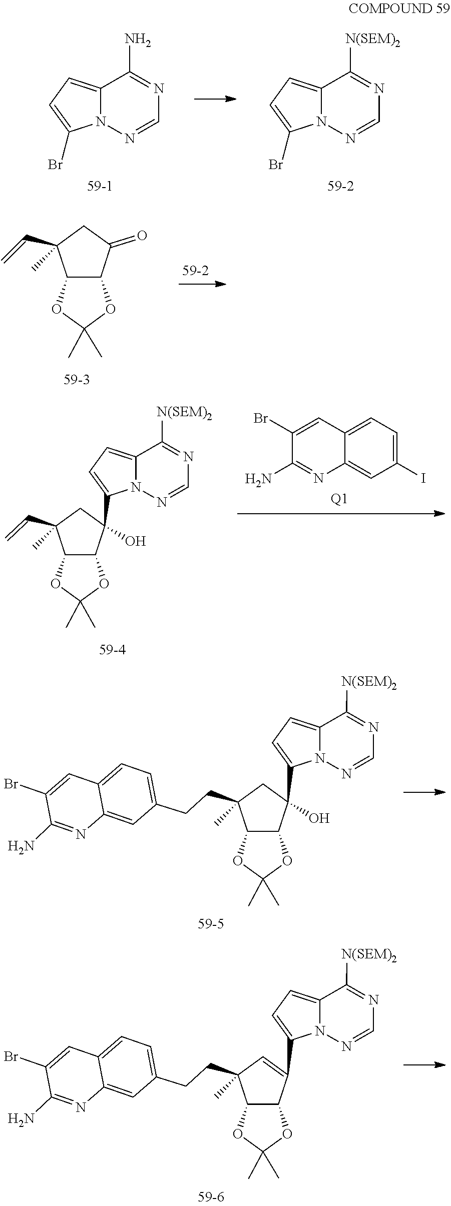

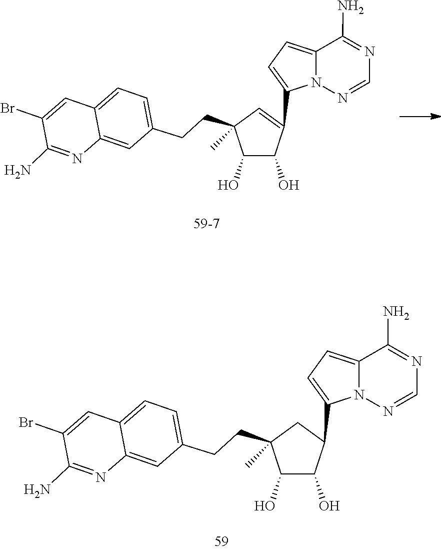

To a solution of 59-1 (2 g, 9.39 mmol, 1 eq.) in DMF (20 mL) was added NaH (938.7 mg, 23.47 mmol, 60% purity, 2.5 eq.) at 0° C., and the mixture was stirred at 0° C. for 0.5 h. SEM-Cl (3.29 g, 19.72 mmol, 3.49 mL, 2.1 eq.) was added at 0° C., and the mixture was stirred at 20° C. for 4 h. The reaction was quenched by the addition of H₂O (60 mL) at 20° C., and then extracted with EA (2×50 mL). The combined organic layers were washed with brine (20 mL), dried over Na₂SO₄, filtered, and concentrated under reduced pressure to give a residue. The residue was purified by flash silica gel chromatography (ISCO®; 40 g SepaFlash® Silica Flash Column, Eluent of 0˜4% Ethyl acetate/Petroleum ether gradient @ 45 mL/min) to afford 59-2 (2.64 g, 5.35 mmol, 57% yield, 96% purity) as a colorless oil. LCMS: (ESI): m/z calcd. for C₁₈H₃₄BrN₄O₂Si₂ 475.13 [M+2+H]⁺, found 475.0.

To a solution of 59-2 (3.62 g, 7.64 mmol, 3 eq.) in THF (15 mL) at −15° C. was added iPrMgBr.LiCl (1.3 M in THF, 5.88 mL, 3 eq.) dropwise. The mixture was stirred at −15° C. for 10 min, then allowed to warm to 0° C., and stirred at 0° C. for 1 h. A solution of 59-3 (500 mg, 2.55 mmol, 1 eq.) in THF (5 mL) was added dropwise at −15° C., and the mixture was stirred at 0° C. for 10 min. The reaction was quenched by the addition of NH₄Cl (sat. aq., 10 mL), diluted with H₂O (40 mL) and extracted with EA (2×50 mL). The combined organic layers were washed with brine (30 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜8% Ethyl acetate/Petroleum ether gradient @ 45 mL/min) to afford 59-4 (1.28 g, 2.08 mmol, 82% yield, 96% purity) as a colorless oil. LCMS: (ESI): m/z calcd. for C₂₉H₅₁N₄O₅Si₂ 591.33 [M+H]⁺, found 591.3.

A mixture of 59-4 (680 mg, 1.10 mmol, 96% purity, 1.0 eq.) and 9-BBN dimer (668.43 mg, 2.76 mmol, 2.5 eq.) in THF (15 mL) was stirred at 50° C. for 1.5 h under N₂, and then cooled to 20° C. A solution of K₃PO₄ (1.17 g, 5.52 mmol, 5 eq.) in H₂O (3 mL) was added, and the mixture was stirred for 0.5 h. Q1 (462.6 mg, 1.33 mmol, 1.2 eq.) and Pd(dppf)Cl₂ (80.8 mg, 0.11 mmol, 0.1 eq.) were added. The mixture was purged with N₂ (3×) and stirred at 60° C. for 2 h. The mixture was diluted with H₂O (30 mL) and extracted with EA (2×50 mL). The combined organic layers were washed with brine (20 mL), dried over Na₂SO₄, filtered, and concentrated under reduced pressure to give a residue. The residue was purified by flash silica gel chromatography (ISCO®; 40 g SepaFlash® Silica Flash Column, Eluent of 0˜31% Ethyl acetate/Petroleum ether gradient @ 45 mL/min) to afford 59-5 (663 mg, 773.8 μmol, 70% yield, 95% purity) as a colorless oil. LCMS: (ESI): m/z calcd. for C₃₈H₅₈BrN₆O₅Si₂815.31 [M+2+H]⁺, found 815.3.

To a solution of 59-5 (663.0 mg, 773.80 μmol, 95% purity, 1 eq.) in DCM (10 mL) was added DAST (623.64 mg, 3.87 mmol, 511.18 μL, 5 eq.) at 0° C., and the mixture was stirred at 0° C. for 1 h. The reaction was quenched by addition sat. NaHCO₃ (2 mL), diluted with H₂O (20 mL) and extracted with EA (2×50 mL). The combined organic layers were washed with brine (10 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜27% Ethyl acetate/Petroleum ether gradient @ 45 mL/min) to afford 59-6 (249 mg, 303.45 μmol, 39% yield, 97% purity) as a white solid. LCMS: (ESI): m/z calcd. for C₃₈H₅₆BrN₆O₄Si₂ 797.30 [M+2+H]⁺, found 797.3.

To a solution of 59-6 (249 mg, 303.45 μmol, 97% purity, 1 eq.) in THF (6 mL) was added HCl (4 M, 2.91 mL, 38.36 eq.) at 20° C. under N₂, and the mixture was stirred at 20° C. for 5 h. The mixture was concentrated under reduced pressure to give a residue. The residue was dissolved in a mixed solvent of H₂O (12 mL) and t-BuOH (12 mL). PPTS (762.57 mg, 3.03 mmol, 10 eq.) was added at 20° C. The mixture was stirred at 50° C. for 12 h, and concentrated under reduced pressure to give a residue. The residue was purified by flash column (C18 column, 0.5% NH₃.H₂O-ACN), and then triturated with EA (2 mL) at 20° C. for 2 h. The solid was collected by filtration and dried under reduced pressure to afford 59-7 (80 mg, 142.12 μmol, 47% yield, 88% purity) as an off-white solid. LCMS: (ESI): m/z calcd. for C₂₃H₂₄BrN₆O₂ 497.11 [M+2+H]⁺, found 497.2.

To a solution of 59-7 (50 mg, 88.8 μmol, 88% purity, 1 eq.) in MeOH (20 mL) was added Crabtree's catalyst (600 mg, 745.44 μmol, 8.39 eq.). The mixture was degassed under vacuum and purged with H₂ several times, and stirred under H₂ (15 psi) at 20° C. for 24 h. TMT (1, 3, 5-triazine-2, 4, 6-trithiol) was added, and the mixture was stirred at 20° C. for 15 min. The insoluble materials were removed by filtration. The filtrate was concentrated under reduced pressure to give a residue. The residue was purified by prep-TLC (SiO₂, DCM:MeOH=10:1) to afford the impure product (13 mg, 23.52 μmol, 26% yield, 90% purity) as a yellow solid, which was combined with other batches to give 19 mg of the impure product. The impure product (19 mg) was purified by prep-HPLC (basic condition, column: Phenomenex Gemini-NX 80*40 mm*3 um; mobile phase: [water (0.05% NH₃H₂O+10 mM NH₄HCO₃)-ACN]; B %: 30%-50%, 8 min) to give 59 (9 mg, 21.06 μmol, 98% purity) as a white solid. TLCMS: (ESI): m/z calcd. for C₂₃H₂₆BrN₆O₂ 499.12 [M+2+H]⁺, found 499.2. ¹H NMR (400 MHz, CD₃OD) δ: 8.25 (s, 1H), 7.76 (s, 1H), 7.55 (d, J=8.3 Hz, 1H), 7.38 (s, 1H), 7.15 (d, J=8.3 Hz, 1H), 6.84 (d, J=4.3 Hz, 1H), 6.54 (d, J=4.3 Hz, 1H), 4.44 (t, J=6.8 Hz, 1H), 3.86 (d, J=6.3 Hz, 1H), 3.69-3.78 (m, 1H), 2.70-2.88 (m, 2H), 2.00-2.09 (m, 2H), 1.72-1.79 (m, 2H), 1.23 (s, 3H).

### Example 55

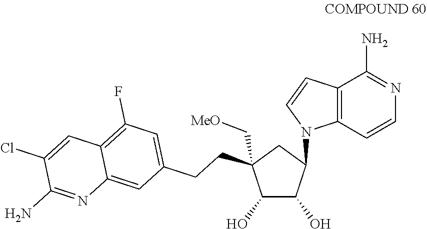 [SMILES: COC[C@]1(CCc2cc(F)c3cc(Cl)c(N)nc3c2)C[C@@H](n2ccc3c(N)nccc32)[C@H](O)[C@@H]1O]

Compound 60 was prepared similarly as described for 58, using 58-8, and using Q16 instead of Q1. ¹H NMR (400 MHz, CD₃OD) δ: 8.21 (s, 1H), 8.19-8.05 (m, 1H), 7.28 (s, 1H), 7.25 (d, J=3.5 Hz, 1H), 6.96 (d, J=11.3 Hz, 1H), 6.61 (d, J=3.5 Hz, 1H), 5.06-4.96 (m, 1H), 4.66 (dd, J=5.3, 9.0 Hz, 1H), 3.99 (d, J=5.3 Hz, 1H), 3.71 (d, J=9.5 Hz, 1H), 3.51 (d, J=9.3 Hz, 1H), 3.45 (s, 3H), 2.91-2.70 (m, 2H), 2.17 (dd, J=9.4, 13.7 Hz, 1H), 2.00 (t, J=8.5 Hz, 2H), 1.91 (dd, J=9.8, 13.6 Hz, 1H); ¹⁹F NMR (376 MHz, CD₃OD) δ: −125.91 (s, 1F).

### Example 56

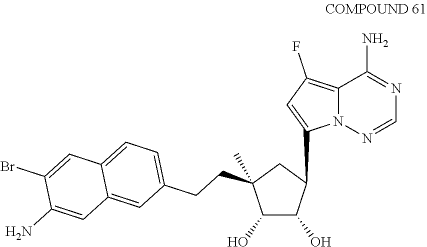 [SMILES: C[C@]1(CCc2ccc3cc(Br)c(N)cc3c2)C[C@@H](c2cc(F)c3c(N)ncnn23)[C@H](O)[C@@H]1O]

Compound 61 was prepared similarly as described for compound 59, using 7-bromo-5-fluoro-N,N-bis((2-(trimethylsilyl)ethoxy)methyl)pyrrolo[2,1-f][1,2,4]triazin-4-amine instead of 7-bromo-N,N-bis((2-(trimethylsilyl)ethoxy)methyl)pyrrolo[2,1-f][1,2,4]triazin-4-amine. ¹H NMR (400 MHz, MeOD) δ: 8.26 (s, 1H), 7.66 (s, 1H), 7.55 (d, J=8.3 Hz, 1H), 7.38 (s, 1H), 7.15 (dd, J=1.4, 8.2 Hz, 1H), 6.34 (s, 1H), 4.42-4.37 (m, 1H), 3.84 (d, J=6.0 Hz, 1H), 3.75-3.67 (m, 1H), 2.87-2.69 (m, 2H), 2.02 (dd, J=8.9, 12.9 Hz, 1H), 1.87-1.66 (m, 3H), 1.21 (s, 3H). ¹⁹F NMR (376 MHz, MeOD) δ: −161.14 (s, 1F).

7-bromo-5-fluoro-N,N-bis((2-(trimethylsilyl)ethoxy)methyl)pyrrolo[2,1-f][1,2,4]triazin-4-amine was prepared as follows. To a solution of 7-bromo-5-fluoro-N,N-bis((2-(trimethylsilyl)ethoxy)methyl)pyrrolo[2,1-f][1,2,4]triazin-4-amine (1 g, 2.11 mmol, 1 eq.) in DMF (10 mL) was added SelectFluor (2.24 g, 6.34 mmol, 3 eq.). The mixture was stirred at 10° C. for 12 h. Upon completion, the mixture was diluted with brine (15 mL) and extracted with EA (3×30 mL). The separated organic layers were combined and dried over anhydrous Na₂SO₄, filtered and concentrated to give a residue. The residue was purified by flash silica gel chromatography (ISCO®; 40 g CombiFlash® Silica Flash Column, Eluent of 0˜5% ethyl acetate/petroleum ether gradient at 50 mL/min) to give an impure product. The impure product was further purified by flash chromatography (40 g, C₁₈ Column, Eluent of 0˜73% ACN/H₂O gradient at 50 mL/min) to give 7-bromo-5-fluoro-N,N-bis((2-(trimethylsilyl)ethoxy)methyl)pyrrolo[2,1-f][1,2,4]triazin-4-amine (190 mg, 0.360 mmol, 17%) as a colorless oil.

The following procedure was used for the synthesis of (3aR,4R,6R,6aR)-4-(4-(bis((2-(trimethylsilyl)ethoxy)methyl)amino)-5-fluoropyrrolo[2,1-f][1,2,4]triazin-7-yl)-2,2,6-trimethyl-6-vinyltetrahydro-4H-cyclopenta[d][1,3]dioxol-4-ol. To a solution of 7-bromo-5-fluoro-N,N-bis((2-(trimethylsilyl)ethoxy)methyl)pyrrolo[2,1-f][1,2,4]triazin-4-amine (541.03 mg, 1.10 mmol, 1.2 eq.) in THF (5 mL) was added dropwise n-BuLi (2.5 M, 0.55 mL, 1.5 eq.) at −78° C. The mixture was stirred at −78° C. for 0.5 h. A solution of (3aR,6R,6aR)-2,2,6-trimethyl-6-vinyltetrahydro-4H-cyclopenta[d][1,3]dioxol-4-one (180 mg, 0.917 mmol, 1 eq.) in THF (5 mL) was added dropwise at −78° C. The mixture was stirred at −78° C. for 5 h. The reaction progress was monitored by TLC (PE:EA=10:1). Upon completion, the reaction was quenched with H₂O (20 mL) and extracted with ethyl acetate (3×20 mL). The combined organic layers were washed with brine (50 mL), dried over anhydrous Na₂SO₄, filtered and concentrated under reduced pressure to give a residue. The residue was purified by flash silica gel chromatography (ISCO®; 40 g CombiFlash® Silica Flash Column, Eluent of 0˜8% ethyl acetate/petroleum ether gradient at 40 mL/min) to give (3aR,4R,6R,6aR)-4-(4-(bis((2-(trimethylsilyl)ethoxy)methyl)amino)-5-fluoropyrrolo[2,1-f][1,2,4]triazin-7-yl)-2,2,6-trimethyl-6-vinyltetrahydro-4H-cyclopenta[d][1,3]dioxol-4-ol (300 mg, 0.483 mmol, 53) as a yellow gum. LCMS: (ESI): m/z calcd. for C₂₉H₄₉O₅N₄Si₂FNa, 631.3, [M+Na]⁺, found 631.3.

### Example 57

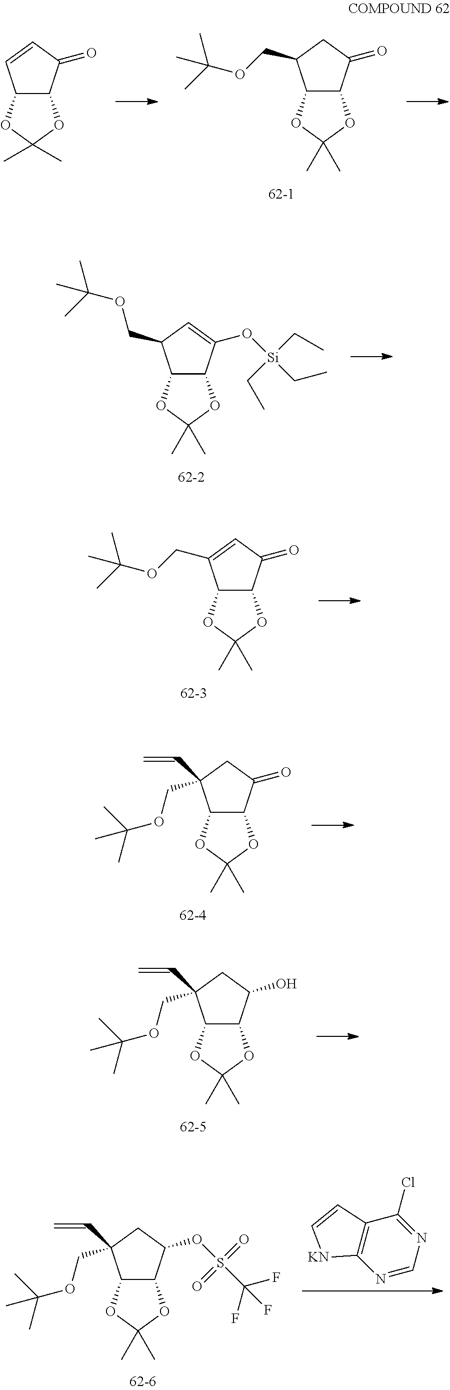

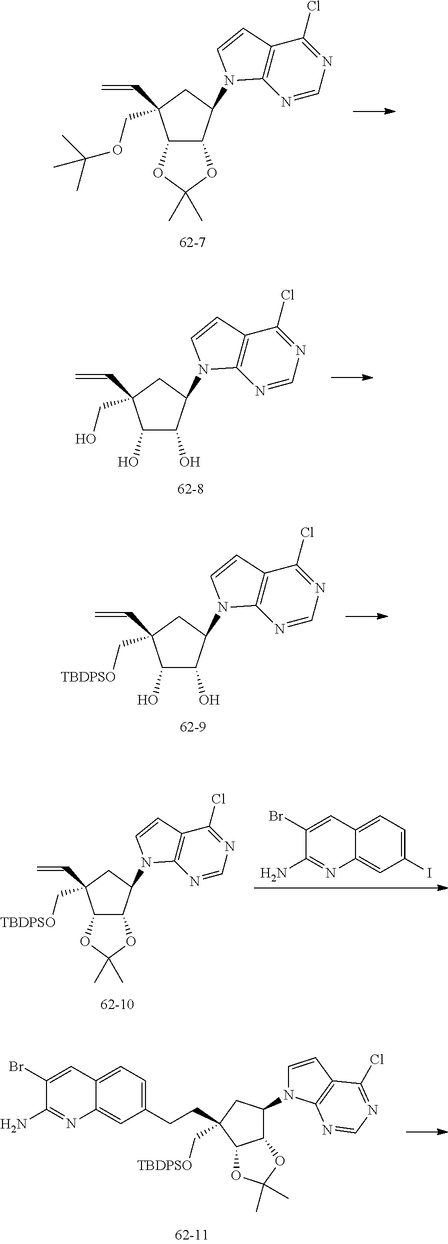

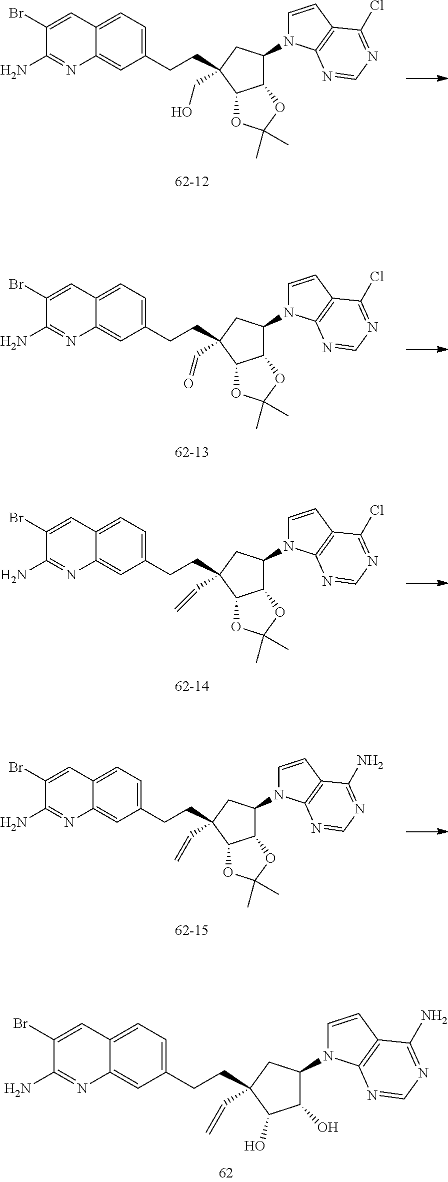 [SMILES: CC1(C)O[C@H]2[C@H](n3ccc4c(Cl)ncnc43)C[C@](CO)(CCc3ccc4cc(Br)c(N)nc4c3)[C@H]2O1 ; CC1(C)O[C@H]2[C@H](n3ccc4c(Cl)ncnc43)C[C@](C=O)(CCc3ccc4cc(Br)c(N)nc4c3)[C@H]2O1 ; C=C[C@]1(CCc2ccc3cc(Br)c(N)nc3c2)C[C@@H](n2ccc3c(Cl)ncnc32)[C@@H]2OC(C)(C)O[C@@H]21 ; C=C[C@]1(CCc2ccc3cc(Br)c(N)nc3c2)C[C@@H](n2ccc3c(N)ncnc32)[C@@H]2OC(C)(C)O[C@@H]21 ; C=C[C@]1(CCc2ccc3cc(Br)c(N)nc3c2)C[C@@H](n2ccc3c(N)ncnc32)[C@H](O)[C@@H]1O]

To a suspension of t-BuOK (10.9 g, 97.3 mmol) in MTBE (225 mL) was added t-BuLi (1.3 M in hexanes, 74.9 mL) dropwise at −70° C. under N₂ atmosphere. The mixture was stirred at −70° C. for 2.5 h. A solution of LiBr (2 M in THF, 97.3 mL) was added dropwise at −70° C., and the mixture was stirred at −10° C. for 0.5 h. A solution of CuBr.Me₂S (10.0 g, 48.7 mmol) in i-Pr₂S (50 mL) was added to the mixture at −70° C., followed by adding a solution of (3aR,6aR)-2,2-dimethyl-3a,6a-dihydro-4H-cyclopenta[d][1,3]dioxol-4-one (5 g, 32.4 mmol) in THL (25 mL). After the mixture was stirred at −10° C. for 0.5 h, the reaction was quenched with a mixture of MeOH:AcOH (1:1, 25 mL) at −10° C., followed by successive addition of NH₄Cl (sat aq. 62.5 mL) and NH₃.H₂O (33% purity, 62.5 mL). The mixture was extracted with EtOAc (2×500 mL). The combined organic layer was washed with brine (500 mL), dried over anhydrous Na₂SO₄, filtered and concentrated under reduced pressure. The residue was purified by silica gel column (1˜10% EA/Pe) to give 62-1 (5.2 g) as a yellow solid. ¹H NMR (400 MHz, CDCl₃) δ: 4.61 (d, J=5.2 Hz, 1H), 4.22 (d, J=5.2 Hz, 1H), 3.53 (dd, J=2.0, 8.4 Hz, 1H), 3.34 (dd, J=2.0, 8.4 Hz, 1H), 2.70 (dd, J=8.8, 18.0 Hz, 1H), 2.53 (d, J=8.4 Hz, 1H), 2.04 (d, J=18.0 Hz, 1H), 1.42 (s, 3H), 1.34 (s, 3H), 1.10 (s, 9H).

To a mixture of 62-1 (5.0 g, 20.6 mmol) and TESCl (7.02 mL, 41.3 mmol) in THF (50 mL) was added LiHMDS (1 M in THF, 31.0 mL) at −78° C. under N₂ atmosphere. The mixture was stirred at −78° C. for 0.5 h. The reaction was quenched with sat aq. NH₄Cl (500 mL) at −78° C. and then extracted with EtOAc (2×250 mL). The combined organic layer was washed with brine (300 mL), dried over anhydrous Na₂SO₄, filtered and concentrated under reduced pressure to give 62-2 (7.15 g, crude) as a yellow oil.

A mixture of 62-2 (7.15 g, crude) and Pd(OAc)₂ (1.35 g, 6.02 mmol) in DMSO (50 mL) was stirred at 60° C. for 12 h under O₂ (15 Psi) atmosphere. The mixture was diluted with water (150 mL) and then extracted with EtOAc (2×100 mL). The combined organic layer was washed with brine (100 mL), dried over anhydrous Na₂SO₄ and concentrated under reduced pressure. The residue was purified by silica gel column (1˜10% EA/PE) to give 62-3 (3.7 g) as a yellow oil. ¹H NMR (400 MHz, CDCl₃) δ: 6.15 (t, J=1.6 Hz, 1H), 5.09 (d, J=6.0 Hz, 1H), 4.49 (d, J=5.6 Hz, 1H), 4.41 (dd, J=1.6, 17.6 Hz, 1H), 4.22 (dd, J=1.2, 18.0 Hz, 1H), 1.40 (s, 3H), 1.39 (s, 3H), 1.24 (s, 9H).

To a suspension of CuI (4.36 g, 22.9 mmol) in THF (150 mL) was added TMEDA (9.42 mL, 62.4 mmol) at 0° C. under N₂ atmosphere. The mixture was stirred at 0° C. for 10 min and cooled down to −78° C. Vinylmagnesium bromide (1 M in THF, 62.4 mL) was added dropwise to the mixture at −78° C. The mixture was stirred at −78° C. for another 20 min. After adding TMSCl (6.34 mL, 49.94 mmol) and 62-3 (10.0 g, 41.62 mmol) to the mixture at −78° C., the mixture was stirred at −78° C. for 3 h. The reaction was quenched with sat aq. NH₄Cl (500 mL) and extracted with EtOAc (2×400 mL). The combined organic layer was washed with brine (400 mL), dried over anhydrous Na₂SO₄, filtered and concentrated under reduced pressure. The residue was purified by silica gel column (1˜10% EA/PE) to provide 62-4 (6.50 g) as a yellow oil. ¹H NMR (400 MHz, CDCl₃) δ: 6.02 (dd, J=11.2, 17.6 Hz, 1H), 5.16 (d, J=11.2 Hz, 1H), 4.99 (d, J=18.0 Hz, 1H), 4.58 (d, J=4.8 Hz, 1H), 4.18 (d, J=5.2 Hz, 1H), 3.62 (d, J=8.4 Hz, 1H), 3.44 (d, J=8.4 Hz, 1H), 2.50 (d, J=18.0 Hz, 1H), 2.38 (d, J=18.0 Hz, 1H), 1.42 (s, 3H), 1.34 (s, 3H), 1.17 (s, 9H).

To a mixture of 62-4 (23.0 g, 85.7 mmol) and CeCl₃ (5.39 mL, 85.7 mmol) in MeOH (700 mL) was added NaBH₄ (4.86 g, 128.56 mmol, 1.5 eq) at −78° C. under N₂ atmosphere. The mixture was stirred at −78° C. for 1 h, then warmed to 20° C., and stirred at 20° C. for 1 h. The reaction was quenched with sat aq. NH₄Cl (500 mL) and filtered through celite. The filtrate was extracted with EtOAc (2×500 mL). The combined organic layer was washed with brine (500 mL), dried over anhydrous Na₂SO₄, filtered and concentrated under reduced pressure. The residue was purified by silica gel column (1˜10% EA/PE) to give 62-5 (19.3 g) as a white solid. ¹H NMR (400 MHz, CDCl₃) δ: 5.83 (dd, J=11.2, 18.0 Hz, 1H), 5.12 (d, J=10.8 Hz, 1H), 5.01 (d, J=18.0 Hz, 1H), 4.50-4.40 (m, 2H), 4.06-3.97 (m, 1H), 3.44 (d, J=8.4 Hz, 1H), 3.30 (d, J=8.4 Hz, 1H), 2.44 (brs, 1H), 2.07-2.00 (m, 1H), 1.49 (s, 3H), 1.48-1.41 (m, 1H), 1.35 (s, 3H), 1.13 (s, 9H).

To a solution of 62-5 (13.3 g, 49.2 mmol) in DCM (250 mL) were added pyridine (16 mL, 197 mmol) and Tf₂O (16.2 mL, 98.4 mmol) dropwise at 0° C. under N₂ atmosphere. The mixture was stirred at 25° C. for 2 h. The mixture was basified with sat aq. NaHCO₃ to pH 7 and extracted with DCM (2×200 mL). The combined organic layer was washed with brine (200 mL), dried over anhydrous Na₂SO₄, filtered and concentrated under reduced pressure. The residue was purified by silica gel column (2% EA/PE) to provide 62-6 (17.8 g) as a yellow oil.

To a solution of 62-6 (11.9 g, 61.92 mmol) in DMF (100 mL) was added a solution of 6-Chloro-7-deazapurine potassium salt (17.8 g, 44.23 mmol) in DMF (20 mL) dropwise at 0° C. under N₂ atmosphere. The mixture was stirred at 20° C. for 12 h. The reaction was quenched with water (500 mL) and extracted with EtOAc (2×500 mL). The combined organic layer was washed with brine (500 mL), dried over anhydrous Na₂SO₄, filtered and concentrated under reduced pressure. The residue was purified by silica gel column (1˜10% EA/PE) to give 62-7 (12.1 g) as a yellow oil. ¹H NMR (400 MHz, CDCl₃) δ: 8.64 (s, 1H), 7.31 (d, J=3.6 Hz, 1H), 6.60 (d, J=4.0 Hz, 1H), 6.05 (dd, J=10.8, 17.6 Hz, 1H), 5.24-5.13 (m, 3H), 5.03 (dd, J=4.4, 6.8 Hz, 1H), 4.77 (d, J=7.2 Hz, 1H), 3.54 (d, J=8.8 Hz, 1H), 3.43 (d, J=9.2 Hz, 1H), 2.66 (dd, J=7.6, 13.2 Hz, 1H), 2.34 (dd, J=10.4, 13.2 Hz, 1H), 1.58 (s, 3H), 1.31 (s, 3H), 1.21 (s, 9H).

A solution of 62-7 (16.3 g, 40.2 mmol) in the mixture of TFA (200 mL) and H₂O (100 mL) was stirred at 50° C. for 8 h under N₂ atmosphere. The mixture was concentrated under reduced pressure. The residue was basified with sat aq. NaHCO₃ to pH 7-8 and extracted with EtOAc (2×300 mL). The combined organic layer was washed with brine (300 mL), dried over anhydrous Na₂SO₄, filtered and concentrated under reduced pressure. The residue was purified by silica gel column (30˜50% EA/PE, 5% methanol/dichloromethane) to give 62-8 (9.1 g) as a white solid. ¹H NMR (400 MHz, CD₃OD) δ: 8.55 (s, 1H), 7.63 (d, J=3.6 Hz, 1H), 6.69 (d, J=4.0 Hz, 1H), 6.09 (dd, J=10.8, 17.6 Hz, 1H), 5.35-5.21 (m, 3H), 4.53 (dd, J=4.8, 8.4 Hz, 1H), 4.12 (d, J=4.4 Hz, 1H), 3.71 (s, 2H), 2.41 (dd, J=10.4, 14.4 Hz, 1H), 2.07 (dd, J=8.4, 13.6 Hz, 1H).

To a mixture of 62-8 (2.8 g, 9.04 mmol) and imidazole (1.85 g, 27.1 mmol) in DMF (9 mL) was added TBDPSCl (3.02 mL, 11.8 mmol) at 0° C. under N₂ atmosphere. The mixture was stirred at 20° C. for 8 h. The reaction was quenched with water (100 mL) and extracted with EtOAc (2×100 mL). The combined organic layer was washed with brine (100 mL), dried over anhydrous Na₂SO₄, filtered and concentrated under reduced pressure. The residue was purified by silica gel column (30% EA/PE, then 10% methanol/dichloromethane) to give 62-9 (2.6 g) as a white solid. LCMS: (ESI): m/z calcd. for C₃₀H₃₅ClN₃O₃Si 548.21 [M+H]⁺, found 548.1. ¹H NMR (400 MHz, CDCl₃) δ: 8.60 (s, 1H), 7.72-7.64 (m, 4H), 7.46-7.36 (m, 6H), 7.31 (d, J=3.6 Hz, 1H), 6.63 (d, J=3.6 Hz, 1H), 6.05 (dd, J=11.2, 17.6 Hz, 1H), 5.27-5.19 (m, 2H), 5.02 (dd, J=8.8, 17.6 Hz, 1H), 4.47-4.41 (m, 1H), 4.29 (t, J=4.8 Hz, 1H), 4.02 (d, J=6.8 Hz, 1H), 3.89 (d, J=10.4 Hz, 1H), 3.79 (d, J=10.0 Hz, 1H), 3.59 (d, J=3.2 Hz, 1H), 2.41 (dd, J=9.6, 14.0 Hz, 1H), 2.10-2.05 (m, 1H), 1.11 (s, 9H).

To a solution of 62-9 (2.60 g, 4.74 mmol) and 2,2-dimethoxypropane (19.2 mL, 156 mmol) in acetone (20 mL) was added p-TsOH (82 mg, 474 μmol) at 0° C. under N₂ atmosphere. The mixture was stirred at 20° C. for 3 h. The mixture was basified with sat aq. NaHCO₃ (50 mL) and extracted with EtOAc (2×100 mL). The combined organic layer was washed with brine (100 mL), dried over anhydrous Na₂SO₄, and concentrated under reduced pressure. The residue was purified by silica gel column (1-50% EA/PE) to give 62-10 (2.5 g) as a yellow oil. ¹H NMR (400 MHz, CDCl₃) δ: 8.64 (s, 1H), 7.76-7.68 (m, 4H), 7.50-7.38 (m, 6H), 7.20 (d, J=4.0 Hz, 1H), 6.59 (d, J=3.6 Hz, 1H), 6.12 (dd, J=10.8, 17.6 Hz, 1H), 5.29-5.18 (m, 2H), 5.02 (dd, J=4.4, 6.8 Hz, 1H), 4.95-4.89 (m, 1H), 4.78 (d, J=6.8 Hz, 1H), 3.83 (d, J=10.0 Hz, 1H), 3.76 (d, J=10.0 Hz, 1H), 2.68 (dd, J=8.4, 13.6 Hz, 1H), 2.38 (dd, J=9.6, 13.2 Hz, 1H), 1.38 (s, 3H), 1.26 (s, 3H), 1.06 (s, 9H).

To a solution of 62-10 (0.7 g, 1.19 mmol) in THF (10 mL) was added 9-BBN dimer (720 mg, 2.98 mmol). The mixture was stirred at 50° C. for 2.5 h under Ar atmosphere. After the mixture was cooled to 20° C., a solution of K₃PO₄ (1.26 g, 5.95 mmol) in H₂O (2 mL) was added to the mixture. The mixture was stirred at rt for 0.5 h. After adding 3-bromo-7-iodoquinolin-2-amine (540 mg, 1.55 mmol) and Pd(dppf)Cl₂ (174 mg, 238 μmol) to the mixture, the mixture was purged with Ar (3×) and stirred at 60° C. for 3 h. The reaction was quenched with water (80 mL) and extracted with EtOAc (80 mL). The combined organic layers were washed with brine (30 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜25% EA/DCM gradient @ 30 mL/min) to give 62-11 (725 mg) as a brown solid. ¹H NMR (400 MHz, CDCl₃) δ: 8.41-8.32 (m, 1H), 7.88 (s, 1H), 7.53-7.41 (m, 4H), 7.26-7.11 (m, 7H), 7.04-6.96 (m, 1H), 6.93-6.80 (m, 2H), 6.33 (dd, J=2.0, 3.3 Hz, 1H), 5.14-5.01 (m, 2H), 4.85 (br t, J=5.5 Hz, 1H), 4.49-4.33 (m, 2H), 3.67 (br d, J=11.0 Hz, 1H), 3.45 (br d, J=10.6 Hz, 1H), 2.61 (br d, J=5.7 Hz, 2H), 2.15-1.94 (m, 2H), 1.41 (br d, J=10.4 Hz, 2H), 1.09 (s, 3H), 0.99 (s, 3H), 0.87 (d, J=1.5 Hz, 9H).

A solution of 62-11 (789 mg, 973 μmol) in TBAF (1 M in THF, 1.95 mL) was stirred at 30° C. for 12 h. The mixture was diluted with water (20 mL) and extracted with EtOAc (3×20 mL). The combined organic layers were washed with brine (15 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜40% EA/PE gradient @ 20 mL/min) to afford 62-12 (410 mg) as a white foam. ¹H NMR (400 MHz, CDCl₃) δ: 8.64 (s, 1H), 8.15 (s, 1H), 7.52-7.50 (m, 2H), 7.28 (d, J=3.5 Hz, 1H), 7.18 (br, d, J=8.4 Hz, 1H), 6.63 (d, J=3.5 Hz, 1H), 5.41 (br s, 2H), 5.25-5.15 (m, 1H), 5.14-5.02 (m, 1H), 4.68 (d, J=7.3 Hz, 1H), 3.98 (br, d, J=11.5 Hz, 1H), 3.80 (br s, 1H), 2.94 (br, dd, J=4.9, 12.6 Hz, 1H), 2.74 (dt, J=4.9, 12.9 Hz, 1H), 2.56-2.44 (m, 1H), 2.39-2.18 (m, 2H), 1.90 (dt, J=4.9, 12.9 Hz, 1H), 1.67-1.61 (m, 3H), 1.34 (s, 3H).

To a solution of 62-12 (300 mg, 524 μmol) in ACN (6 mL) was added IBX (293.27 mg, 1.05 mmol), and the mixture was stirred at 65° C. for 2 h. The mixture was filtered and concentrated under reduced pressure to give 62-13 (350 mg, crude) as a yellow foam.

To a solution of MePPh₃Br (469 mg, 1.31 mmol) in THF (1.5 mL) was added tBuOK (124 mg, 1.05 mmol) in portions at 0° C., and the mixture was stirred at 0° C. for 1 h. After adding the solution of 62-13 (300 mg, crude) in THF (3 mL) dropwise into the mixture, the mixture was stirred at 10° C. for 12 h. The reaction was quenched by NH₄Cl (sat., aq., 10 mL), diluted with water (5 mL), and extracted with EtOAc (3×20 mL). The combined organic layer was washed with brine (10 mL), dried over Na₂SO₄, filtered, and concentrated under reduced pressure to give a residue. The residue was purified by flash silica gel chromatography (ISCO®; 12 g SepaFlash® Silica Flash Column, Eluent of 0˜60% EA/dichloromethane @ 25 mL/min) to afford 62-14 (265 mg) as a white foam. LCMS: (ESI): m/z calcd. for C₂₇H₂₈BrClN₅O₂ 570.10. [M+2+H]⁺, found 570.3. ¹H NMR (400 MHz, CDCl₃) δ: 8.65 (s, 1H), 8.15 (s, 1H), 7.54-7.42 (m, 2H), 7.30 (br d, J=3.3 Hz, 1H), 7.12 (d, J=7.8 Hz, 1H), 6.63 (d, J=3.8 Hz, 1H), 6.24 (dd, J=11.0, 17.8 Hz, 1H), 5.59-5.24 (m, 4H), 5.16-5.11 (m, 1H), 5.08-5.01 (m, 1H), 4.69 (d, J=7.5 Hz, 1H), 2.80 (br s, 1H), 2.65 (br dd, J=7.0, 13.1 Hz, 2H), 2.56-2.47 (m, 1H), 1.97 (br dd, J=5.1, 12.2 Hz, 2H), 1.59 (s, 3H), 1.32 (s, 3H).

To a solution of 62-14 (265 mg, 466 μmol) in dioxane (4 mL) was added NH₃.H₂O (4 mL), and the mixture was stirred at 100° C. for 12 h in a sealed tube. The mixture was cooled to rt, diluted with water (10 mL), and extracted with EtOAc (3×20 mL). The combined organic layers were washed with brine (20 mL), dried over Na₂SO₄, filtered and concentrated under reduced pressure to give a residue. The residue was purified by column chromatography (SiO₂, PE:EA=3:1 to 1:1, to dichloromethane:methanol=10:1) to afford 62-15 (230 mg, 98% purity) as a light-yellow foam. ¹H NMR (400 MHz, CDCl₃) δ: 8.32 (s, 1H), 8.13 (s, 1H), 7.48 (d, J=8.3 Hz, 1H), 7.41 (s, 1H), 7.10 (d, J=8.0 Hz, 1H), 7.04 (d, J=3.5 Hz, 1H), 6.43 (d, J=3.5 Hz, 1H), 6.22 (dd, J=11.0, 18.1 Hz, 1H), 5.33 (br d, J=5.5 Hz, 2H), 5.19-5.14 (m, 1H), 5.05-4.97 (m, 1H), 4.66 (d, J=7.5 Hz, 1H), 2.77 (br s, 1H), 2.66-2.57 (m, 2H), 2.55-2.47 (m, 1H), 2.00-1.91 (m, 2H), 1.58 (s, 3H), 1.32 (s, 3H).

To a solution of 62-15 (215 mg, 391.29 μmol, 1 eq.) in THF (2 mL) was added HCl (4 M, 1.95 mL, 19.98 eq.), and the mixture was stirred at 20° C. for 2 h. The reaction progress was monitored by LC-MS. Upon completion, the mixture was concentrated under reduced pressure to give a residue. The residue was purified by prep-HPLC (column: Phenomenex Gemini-NX 80*40 mm*3 um; mobile phase: [water (0.05% NH₃H₂O)-ACN]; B %: 23%-53%, 8 min) to afford 62 (135 mg, 98% purity) as a white solid. LCMS: (ESI): m/z calcd. for C₂₄H₂₆BrN₆O₂ 508.12. [M+H]⁺, found 509.0. ¹H NMR (400 MHz, CD₃OD) δ: =8.24 (s, 1H), 8.08 (s, 1H), 7.54 (d, J=8.3 Hz, 1H), 7.37 (s, 1H), 7.23 (d, J=3.5 Hz, 1H), 7.14 (dd, J=1.3, 8.3 Hz, 1H), 6.60 (d, J=3.8 Hz, 1H), 6.14 (dd, J=11.0, 17.8 Hz, 1H), 5.37-5.27 (m, 2H), 5.02-4.94 (m, 1H), 4.63-4.56 (m, 1H), 4.02 (d, J=6.0 Hz, 1H), 2.79-2.70 (m, 1H), 2.70-2.62 (m, 1H), 2.57 (dd, J=9.0, 13.6 Hz, 1H), 2.07-1.95 (m, 3H).

### Example 58

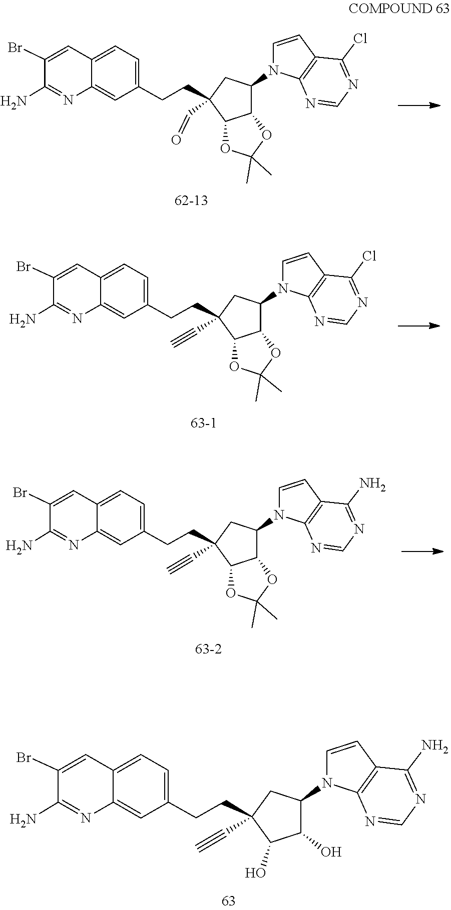 [SMILES: CC1(C)O[C@H]2[C@H](n3ccc4c(Cl)ncnc43)C[C@](C=O)(CCc3ccc4cc(Br)c(N)nc4c3)[C@H]2O1 ; C#C[C@]1(CCc2ccc3cc(Br)c(N)nc3c2)C[C@@H](n2ccc3c(Cl)ncnc32)[C@@H]2OC(C)(C)O[C@@H]21 ; C#C[C@]1(CCc2ccc3cc(Br)c(N)nc3c2)C[C@@H](n2ccc3c(N)ncnc32)[C@@H]2OC(C)(C)O[C@@H]21 ; C#C[C@]1(CCc2ccc3cc(Br)c(N)nc3c2)C[C@@H](n2ccc3c(N)ncnc32)[C@H](O)[C@@H]1O]

To a solution of 62-13 (467 mg, 818 μmol) in ACN (7 mL) and MeOH (7 mL) were added K₂CO₃ (226 mg, 1.64 mmol.) and 1-diazo-1-dimethoxyphosphoryl-propan-2-one (314 mg, 1.64 mmol.). The mixture was stirred at 20° C. for 2 h. The reaction was quenched by NH₄Cl (sat. aq., 20 mL) at 0° C., and extracted with EtOAc (3×20 mL). The combined organic layers were washed with brine (50 mL) and dried over anhydrous Na₂SO₄. The resulting solution was concentrated under reduced pressure. The residue was purified by flash silica gel chromatography (ISCO®; 20 g SepaFlash® Silica Flash Column, Eluent of 10˜60% EA/PE gradient @ 30 mL/min) to give 63-1 (337 mg) as a white foam. ¹H NMR (400 MHz, CDCl₃) δ: 8.63 (s, 1H), 8.12 (s, 1H), 7.54-7.46 (m, 2H), 7.30 (d, J=3.7 Hz, 1H), 7.18 (dd, J=1.5, 8.3 Hz, 1H), 6.61 (d, J=3.6 Hz, 1H), 5.24-5.19 (m, 3H), 4.62 (d, J=6.9 Hz, 1H), 3.80 (d, J=11.8 Hz, 1H), 3.11-2.95 (m, 2H), 2.80-2.70 (m, 1H), 2.62-2.50 (m, 2H), 2.19-2.02 (m, 2H), 1.70 (s, 3H), 1.33 (s, 3H).

To a solution of 63-1 (385 mg, 679 μmol) in dioxane (7 mL) was added NH₃.H₂O (7 mL), and the mixture was stirred in a seated tube at 100° C. for 12 h. The mixture was extracted with EtOAc (3×20 mL). The combined organic layers were washed with brine (20 mL) and dried over anhydrous Na₂SO₄. The resulting solution was concentrated under reduced pressure. The residue was purified by flash silica gel chromatography (ISCO®; 20 g SepaFlash® Silica Flash Column, Eluent of 0˜6% MeOH/DCM gradient @ 30 mL/min) to give 63-2 (268 mg, 97% purity) as a white foam. ¹H NMR (400 MHz, CDCl₃) δ: 8.31 (s, 1H), 8.11 (s, 1H), 7.56-7.42 (m, 2H), 7.17 (dd, J=1.4, 8.4 Hz, 1H), 7.02 (d, J=3.5 Hz, 1H), 6.34 (d, J=3.8 Hz, 1H), 5.28-5.20 (m, 3H), 5.17-5.06 (m, 3H), 4.62 (d, J=7.5 Hz, 1H), 3.13-2.92 (m, 2H), 2.78 (t, J=12.3 Hz, 1H), 2.63-2.44 (m, 2H), 2.19-2.04 (m, 2H), 1.70 (s, 3H), 1.33 (s, 3H).

To a solution of 63-2 (245 mg, 448 μmol) in THF (6 mL) was added HCl solution (4 M, 6 mL), and the mixture was stirred at 20° C. for 12 h. The mixture was concentrated under reduced pressure. The residue was basified by NH₃.H₂O (28%, 1 drop), and further concentrated under reduced pressure. The residue was purified by prep-HPLC (column: Phenomenex Gemini-NX 80*40 mm*3 um; mobile phase: [water (0.05% NH₃.H₂O)-ACN]; B %: 24%-54%, 8 min) to give 63 (135 mg, 99% purity) as a white solid. ¹H NMR (400 MHz, CD₃OD) δ: 8.26 (s, 1H), 8.08 (s, 1H), 7.56 (d, J=8.3 Hz, 1H), 7.42 (s, 1H), 7.26-7.13 (m, 2H), 6.59 (d, J=3.7 Hz, 1H), 5.18-5.01 (m, 1H), 4.54 (t, J=6.9 Hz, 1H), 4.00 (d, J=6.8 Hz, 1H), 3.08 (dt, J=4.7, 12.8 Hz, 1H), 2.93 (dt, J=5.1, 12.7 Hz, 1H), 2.81 (s, 1H), 2.46 (dd, J=8.3, 12.8 Hz, 1H), 2.22-2.11 (m, 2H), 2.02 (dt, J=5.2, 12.5 Hz, 1H).

### Example 59

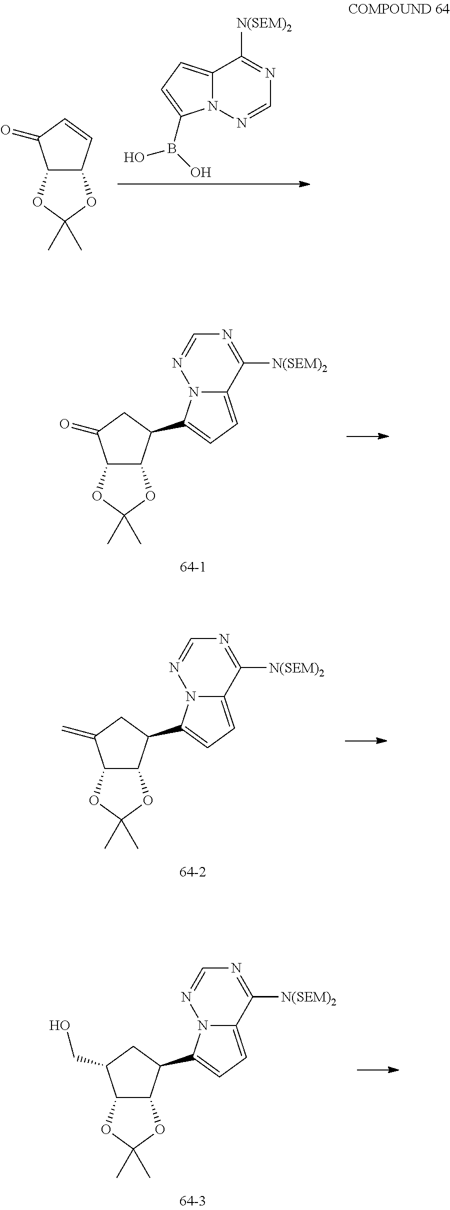

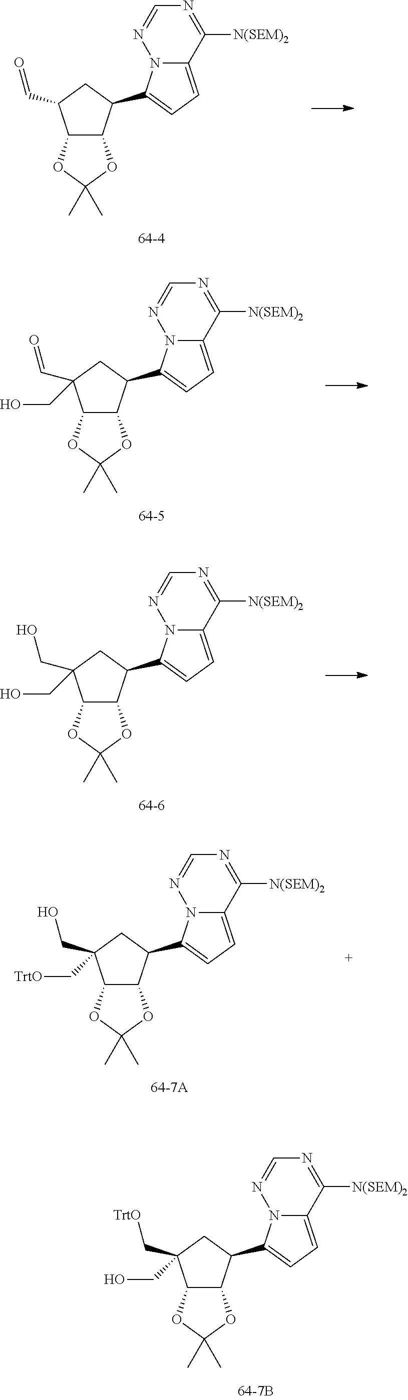

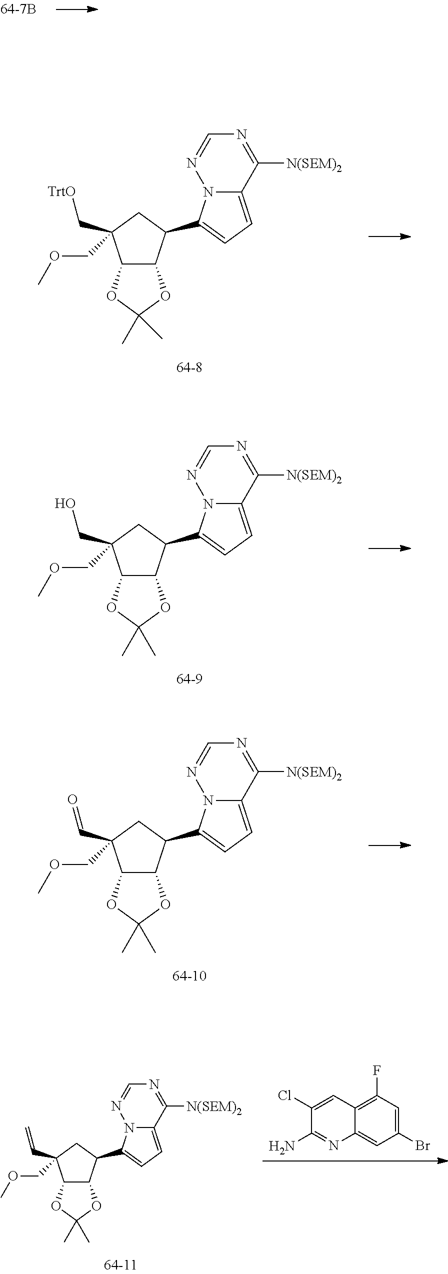

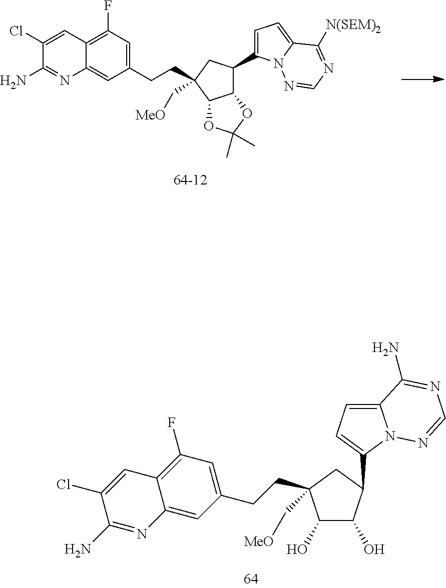

To a solution of (+)-(3aS,6aS)-3a,6a-Dihydro-2,2-dimethyl-4H-cyclopenta-1,3-dioxol-4-one (6.65 g, 43.1 mmol) and B-[4-[bis[[2-(trimethylsilyl)ethoxy]methyl]amino]pyrrolo[2,1-f][1,2,4]triazin-7-yl]-Boronic acid (CAS 2088371-57-5, 25.9 g, 59.0 mmol) in dioxane (110 mL) and H₂O (5 mL) were added BINAP (2.69 g, 4.31 mmol), [Rh(COD)Cl]₂ (638 mg, 1.29 mmol) and Na₂CO₃ (9.14 g, 86.3 mmol.). The mixture was stirred at 100° C. for 0.5 h. After cooling the mixture to rt, the mixture was filtered and concentrated under reduced pressure. The residue was purified by flash silica gel chromatography (ISCO®; 330 g SepaFlash® Silica Flash Column, Eluent of 0˜12% EA/PE gradient @ 100 mL/min) to give 64-1 (21.7 g, 99% purity) as a yellow oil. LCMS: (ESI): m/z calcd. for C₂₆H₄₅N₄O₅Si₂ 549.29 [M+H]⁺, found 549.2. ¹H NMR (400 MHZ, CDCl₃) δ: 7.95 (s, 1H), 6.94 (d, J=4.6 Hz, 1H), 6.49 (d, J=4.6 Hz, 1H), 5.22 (s, 4H), 4.84 (d, J=5.5 Hz, 1H), 4.53 (d, J=5.4 Hz, 1H), 4.10 (br d, J=8.2 Hz, 1H), 3.79-3.59 (m, 4H), 3.11 (dd, J=9.9, 18.7 Hz, 1H), 2.58 (br d, J=18.6 Hz, 1H), 1.51 (s, 3H), 1.35 (s, 3H), 1.08-0.89 (m, 4H), 0.02 (s, 18H).

To a solution of MePPh₃Br (3.25 g, 9.11 mmol) in THF (20 mL) was added t-BuOK (818 mg, 7.29 mmol) at 0° C., and the mixture was stirred at 0° C. for 1 h. A solution of 64-1 (2 g, 3.64 mmol) in THF (10 mL) was added dropwise into the mixture at 0° C. The mixture was stirred at 20° C. for 12 h. The reaction was quenched by addition of NH₄Cl (sat. aq., 20 mL) at 0° C., and extracted with EtOAc (3×20 mL). The combined organic layers were washed with brine (50 mL), dried over anhydrous Na₂SO₄ and concentrated under reduced pressure. The residue was purified by flash silica gel chromatography (ISCO®; 20 g SepaFlash® Silica Flash Column, Eluent of 0˜12% EA/PE gradient @ 25 mL/min) to give 64-2 (1.38 g, 98% purity) as a colorless oil. LCMS: (ESI): m/z calcd. for C₂₇H₄₇N₄O₄Si₂ 547.31 [M+H]⁺, found 547.1. ¹H NMR (400 MHz, CDCl₃) δ: 8.04 (s, 1H), 6.91 (d, J=4.6 Hz, 1H), 6.47 (d, J=4.6 Hz, 1H), 5.30 (br s, 1H), 5.25-5.17 (m, 5H), 4.83-4.73 (m, 2H), 3.96 (br d, J=7.7 Hz, 1H), 3.75-3.58 (m, 4H), 3.25-3.12 (m, 1H), 2.54 (br d, J=15.3 Hz, 1H), 1.55 (s, 3H), 1.35 (s, 3H), 1.05-0.95 (m, 4H), 0.02 (s, 18H).

To a solution of 64-2 (1.89 g, 3.46 mmol) in THF (20 mL) was added 9-BBN dimer (1.25 g, 5.18 mmol) at 0° C., and the mixture was stirred at 20° C. for 2 h. NaOH (3 M, aq., 5.76 mL, 5 eq.) and H₂O₂ (3.92 g, 34.56 mmol, 3.32 mL, 30% purity, 10 eq.) were added to the mixture at 0° C. The mixture was stirred at 20° C. for 2 h. The reaction was quenched by addition of NaS₂O₃ (sat., aq., 50 mL) at 0° C., and extracted with EtOAc (3×50 mL). The combined organic layers were washed with brine (50 mL), dried over anhydrous Na₂SO₄ and concentrated under reduced pressure. The residue was purified by flash silica gel chromatography (ISCO®; 24 g SepaFlash® Silica Flash Column, Eluent of 0˜20% EA/PE gradient @ 25 mL/min) to give 64-3 (1.9 g, 96% purity) as a colorless oil. LCMS: (ESI): m/z calcd. for C₂₇H₄₉N₄O₅Si₂ 565.32 [M+H]⁺, found 565.7. ¹H NMR (400 MHz, CDCl₃) δ: 8.07 (s, 1H), 6.99 (br d, J=4.3 Hz, 1H), 6.46 (d, J=4.5 Hz, 1H), 5.24 (s, 4H), 4.86 (d, J=5.5 Hz, 1H), 4.83-4.76 (m, 1H), 3.95-3.80 (m, 3H), 3.74-3.64 (m, 4H), 2.44-2.21 (m, 3H), 1.55 (s, 3H), 1.35 (s, 3H), 1.04-0.95 (m, 4H), 0.02 (s, 18H).

To a mixture of 64-3 (1.9 g, 3.36 mmol), NMO (816 μL, 7.74 mmol), and 4 Å MS (2 g) in DCM (38 mL) was added TPAP (118 mg, 336 μmol) at 0° C., and the mixture was stirred at 0° C. for 1 h. The mixture was filtered and concentrated under reduced pressure. The residue was purified by flash silica gel chromatography (ISCO®; 40 g SepaFlash® Silica Flash Column, Eluent of 0˜25% EA/PE ether gradient @ 30 mL/min) to give 64-4 (1.52 g, 98% purity) as a colorless oil. LCMS: (ESI): m/z calcd. for C₂₇H₄₇N₄O₅Si₂ 563.30 [M+H]⁺, found 563.3. ¹H NMR (400 MHz, CDCl₃) δ: 9.85 (s, 1H), 8.09-8.02 (s, 1H), 6.98 (d, J=4.5 Hz, 1H), 6.42 (d, J=4.5 Hz, 1H), 5.23 (s, 4H), 5.06 (t, J=5.6 Hz, 1H), 4.93 (d, J=5.5 Hz, 1H), 3.94 (d, J=7.3 Hz, 1H), 3.74-3.63 (m, 4H), 3.05 (td, J=6.0, 12.0 Hz, 1H), 2.65 (dt, J=7.4, 12.7 Hz, 1H), 2.15 (dd, J=6.5, 13.3 Hz, 1H), 1.51 (s, 3H), 1.34 (s, 3H), 1.03-0.97 (m, 4H), 0.02 (s, 18H).

To a solution of 64-4 (1.4 g, 2.49 mmol) and HCHO (37% aq. solution, 1.85 mL, 24.87 mmol) in dioxane (15 mL) was added KOH (279.11 mg, 4.97 mmol, 2 eq.) at 0° C., and the mixture was stirred at 20° C. for 2.5 h. The mixture containing 64-5 was directly used for the next step.

The above-solution of crude 64-5 in dioxane was diluted with EtOH (15 mL), followed by the addition of NaBH₄ (281 mg, 7.44 mmol.) at 0° C. The mixture was stirred at 20° C. for 1 h. The reaction was quenched with water (30 mL) and extracted with EtOAc (3×30 mL). The combined organic phase was washed with brine (50 mL), dried over anhydrous Na₂SO₄ and concentrated under reduced pressure. The residue was purified by flash silica gel chromatography (ISCO®; 20 g SepaFlash® Silica Flash Column, Eluent of 0˜50% EA/PE gradient @ 30 mL/min) to give 64-6 (0.75 g, 97% purity) as a colorless oil. LCMS: (ESI): m/z calcd. for C₂₈H₅₁N₄O₆Si₂ 595.33 [M+H]⁺, found 595.3. ¹H NMR (400 MHz, CDCl₃) δ: 8.01 (s, 1H), 7.03 (br s, 1H), 6.64 (d, J=4.8 Hz, 1H), 5.24 (s, 4H), 5.06 (t, J=6.0 Hz, 1H), 4.79 (d, J=6.5 Hz, 1H), 4.20-4.09 (m, 1H), 3.88-3.74 (m, 3H), 3.73-3.61 (m, 5H), 2.14-2.05 (m, 1H), 1.88 (dd, J=9.9, 13.7 Hz, 1H), 1.61 (s, 3H), 1.37 (s, 3H), 1.04-0.95 (m, 4H), 0.02 (s, 18H).

To a solution of 64-6 (0.8 g, 1.34 mmol) in DCM (8 mL) were added TEA (562 μL, 4.03 mmol) and TrtCl (375 mg, 1.34 mmol) at 0° C., and the mixture was stirred at 20° C. for 12 h. The reaction was quenched with water (20 mL) and extracted with DCM (3×10 mL). The combined organic phase was washed with brine (30 mL), dried over anhydrous Na₂SO₄ and concentrated under reduced pressure. The residue was purified by column chromatography (SiO₂, DCM:MTBE=50:1 to 40:1) to give 64-7A (600 mg) as a white solid, and 64-7B (190 mg, 98% purity) as a white solid.

64-7A: LCMS: (ESI): m/z calcd. for C₄₇H₆₅N₄O₆Si₂ 837.44 [M+H]⁺, found 837.5. ¹H NMR (400 MHz, CDCl₃) δ: 8.02 (s, 1H), 7.51 (m, 6H), 7.35-7.30 (m, 7H), 7.26-7.23 (m, 2H), 7.06-6.99 (m, 1H), 6.70-6.63 (m, 1H), 5.24 (s, 4H), 5.17-5.10 (m, 1H), 4.98 (br t, J=5.2 Hz, 1H), 4.60 (br d, J=6.2 Hz, 1H), 3.73-3.65 (m, 4H), 3.62-3.53 (m, 3H), 3.47 (br, t, J=8.3 Hz, 1H), 3.26-3.20 (m, 1H), 2.60 (br s, 1H), 2.38 (br, dd, J=9.0, 13.9 Hz, 1H), 1.98 (br dd, J=7.7, 13.5 Hz, 1H), 1.40 (s, 3H), 1.32 (s, 3H), 1.04-0.96 (m, 4H), 0.02 (s, 18H).

64-7B: LCMS: (ESI): m/z calcd. for C₄₇H₆₅N₄O₆Si₂ 837.44 [M+H]⁺, found 837.4; ¹H NMR (400 MHz, CDCl₃) δ: 7.92 (s, 1H), 7.41 (m, 6H), 7.30-7.16 (m, 9H), 6.91 (br d, J=4.6 Hz, 1H), 6.42 (d, J=4.6 Hz, 1H), 5.25-5.15 (m, 4H), 4.93 (t, J=6.3 Hz, 1H), 4.46 (d, J=6.6 Hz, 1H), 4.00-3.91 (m, 1H), 3.87-3.76 (m, 2H), 3.72-3.60 (m, 4H), 3.34 (d, J=8.8 Hz, 1H), 3.14 (d, J=9.0 Hz, 1H), 2.60 (dd, J=5.0, 8.5 Hz, 1H), 2.26 (dd, J=8.3, 13.6 Hz, 1H), 2.11 (dd, J=10.6, 13.7 Hz, 1H), 1.55 (s, 3H), 1.26 (s, 3H), 1.03-0.93 (m, 4H), 0.00 (s, 18H).

To a solution of 64-7B (195 mg, 233 μmol) in THF (2 mL) was added NaH (28 mg, 699 μmol, 60% purity) at 0° C., and the mixture was stirred at 0° C. for 0.5 h. After adding Mel (73 μL, 1.16 mmol) into the mixture at 0° C., the mixture was stirred at 20° C. for 12 h. The reaction was quenched by NH₄Cl (sat., aq., 10 mL) and extracted with EtOAc (3×10 mL). The combined organic layers were washed with brine (20 mL) and dried over anhydrous Na₂SO₄. The resulting solution was concentrated under reduced pressure. The residue was purified by flash silica gel chromatography (ISCO®; 4 g SepaFlash® Silica Flash Column, Eluent of 0˜10% EA/PE gradient @ 20 mL/min) to give 64-8 (164 mg, 99% purity) as a white solid. LCMS: (ESI): m/z calcd. for C₄₈H₆₇N₄O₆Si₂ 851.45 [M+H]⁺, found 851.9. ¹H NMR (400 MHz, CDCl₃) δ: 7.99 (s, 1H), 7.43 (d, J=7.5 Hz, 6H), 7.32-7.27 (m, 5H), 7.26-7.20 (m, 4H), 6.97 (br s, 1H), 6.28 (br d, J=4.3 Hz, 1H), 5.31-5.20 (m, 4H), 4.75 (t, J=5.6 Hz, 1H), 4.49 (d, J=6.3 Hz, 1H), 3.84 (d, J=9.0 Hz, 1H), 3.81-3.76 (m, 1H), 3.74-3.67 (m, 4H), 3.63-3.56 (m, 1H), 3.42 (s, 3H), 3.24 (d, J=8.8 Hz, 1H), 2.97 (d, J=8.8 Hz, 1H), 2.38 (dd, J=8.8, 13.6 Hz, 1H), 1.91 (br dd, J=8.4, 13.7 Hz, 1H), 1.55 (s, 3H), 1.30 (s, 3H), 1.04-0.98 (m, 4H), 0.03 (s, 18H).

To a solution of 64-8 (110 mg, 129 μmol) in DCM (2.2 mL) were added Et₃SiH (206 μL, 1.29 mmol) and TFA (57 μL, 775 μmol) at 0° C., and the mixture was stirred at 0° C. for 2.5 h. The reaction was quenched by NaHCO₃ (sat., aq., 20 mL) and extracted with EtOAc (2×10 mL). The combined organic layers were washed with brine (20 mL) and dried over anhydrous Na₂SO₄. The resulting solution was concentrated under reduced pressure. The residue was purified by flash silica gel chromatography (ISCO®; 4 g SepaFlash® Silica Flash Column, Eluent of 5˜25% EA/PE gradient @ 20 mL/min) to give 64-9 (70 mg, 93% purity) as a colorless oil. LCMS: (ESI): m/z calcd. for C₂₉H₅₃N₄O₆Si₂ 609.34 [M+H]⁺, found 609.6. ¹H NMR (400 MHz, CDCl₃) δ: 8.05 (s, 1H), 7.05 (br, s, 1H), 6.75 (br, d, J=4.0 Hz, 1H), 5.25 (s, 4H), 5.14 (d, J=4.5 Hz, 1H), 5.01-4.96 (m, 1H), 4.53 (d, J=6.0 Hz, 1H), 3.89-3.83 (m, 1H), 3.79 (d, J=9.3 Hz, 1H), 3.74-3.64 (m, 5H), 3.59 (d, J=9.3 Hz, 1H), 3.43 (s, 3H), 3.15 (br s, 1H), 2.33 (dd, J=8.9, 13.7 Hz, 1H), 2.06 (dd, J=6.8, 13.6 Hz, 1H), 1.57 (s, 3H), 1.34 (s, 3H), 1.04-0.96 (m, 4H), 0.02 (s, 18H).

To a solution of 64-9 (0.1 g, 164 μmol) in ACN (3 mL) was added IBX (92 mg, 328 μmol), and the mixture was stirred at 65° C. for 2 h. The mixture was cooled to rt, filtered and concentrated under reduced pressure. The residue was purified by flash silica gel chromatography (ISCO®; 4 g SepaFlash® Silica Flash Column, Eluent of 0˜15% EA/PE gradient @18 mL/min) to give 64-10 (90 mg, 96% purity) as a colorless oil. LCMS: (ESI): m/z calcd. for C₂₉H₅₁N₄O₆Si₂ 607.33 [M+H]⁺, found 607.4. ¹H NMR (400 MHz, CDCl₃) δ: 9.39 (s, 1H), 8.01 (s, 1H), 6.94 (br d, J=4.5 Hz, 1H), 6.36 (d, J=4.8 Hz, 1H), 5.27-5.16 (m, 4H), 5.12 (d, J=6.0 Hz, 1H), 5.04 (dd, J=2.0, 5.8 Hz, 1H), 3.88 (br, t, J=5.4 Hz, 1H), 3.80 (d, J=9.3 Hz, 1H), 3.72-3.61 (m, 5H), 3.34 (s, 3H), 2.39 (d, J=6.3 Hz, 2H), 1.57 (s, 3H), 1.39 (s, 3H), 1.08-0.95 (m, 4H), 0.02 (s, 18H).

To a solution of MePh₃Br (132 mg, 370.74 μmol) in THF (2 mL) was added t-BuOK (33.3 mg, 297 μmol) at 0° C., and the mixture was stirred at 0° C. for 1 h. A solution of 64-10 (90 mg, 148 μmol) in THF (1 mL) was added into the mixture at 0° C. The mixture was stirred at 20° C. for 12 h. The reaction was quenched by NH₄Cl (sat., aq., 10 mL) at 0° C., and extracted with EtOAc (3×10 mL). The combined organic layers were washed with brine (20 mL), dried over anhydrous Na₂SO₄ and concentrated under reduced pressure. The residue was purified by flash silica gel chromatography (ISCO®; 4 g SepaFlash® Silica Flash Column, Eluent of 0˜15% EA/PE gradient @ 20 mL/min) to give 64-11(80 mg, 95% purity) as a colorless oil. LCMS: (ESI): m/z calcd. for C₃₀H₅₃N₄O₅Si₂ 605.35 [M+H]⁺, found 605.8. ¹H NMR (400 MHz, CDCl₃) δ: 8.00 (s, 1H), 6.94 (d, J=4.8 Hz, 1H), 6.55 (d, J=4.8 Hz, 1H), 5.94 (dd, J=11.0, 17.6 Hz, 1H), 5.22 (s, 4H), 5.12-4.98 (m, 3H), 4.66 (d, J=6.5 Hz, 1H), 3.88 (dt, J=4.3, 8.2 Hz, 1H), 3.73-3.64 (m, 4H), 3.58 (q, J=9.3 Hz, 2H), 3.41 (s, 3H), 2.49 (dd, J=8.3, 13.3 Hz, 1H), 2.12 (dd, J=8.3, 13.3 Hz, 1H), 1.57 (s, 3H), 1.35 (s, 3H), 1.05-0.93 (m, 4H), 0.02 (s, 18H).

To a solution of 64-11 (80 mg, 132 μmol) in THF (2 mL) was added 9-BBN dimer (80.0 mg, 331 μmol), and the mixture was stirred at 50° C. for 2.5 h under Ar atmosphere. After the mixture was cooled to 20° C., a solution of K₃PO₄ (140 mg, 661 μmol) in H₂O (0.2 mL) was added into the mixture. After the mixture was further stirred for 0.5 h at 20° C. 7-bromo-3-chloro-5-fluoroquinolin-2-amine (47.4 mg, 172 μmol) and Pd(dppf)Cl₂ (9.7 mg, 13 μmol) were added to the mixture. The mixture was degassed and purged with Ar (3×) and stirred at 70° C. for 12 h. The mixture was diluted with EtOAc (5 mL) and H₂O (5 mL), and extracted with EtOAc (2×10 mL). The combined organic layers were washed with brine (20 mL) and dried over anhydrous Na₂SO₄. The resulting solution was concentrated under reduced pressure. The residue was purified by flash silica gel chromatography (ISCO®; 4 g SepaFlash® Silica Flash Column, Eluent of 0˜30% EA/PE gradient @18 mL/min) to give 64-12 (66 mg, 96% purity) as a yellow oil. LCMS: (ESI): m/z calcd. for C₃₉H₅₉ClFN₆O₅Si₂ 801.37 [M+H]⁺, found 801.3. ¹H NMR (400 MHz, CD₃OD) δ: 8.14 (s, 1H), 7.99 (s, 1H), 7.16 (s, 1H), 6.97 (d, J=4.5 Hz, 1H), 6.69 (d, J=10.8 Hz, 1H), 6.59 (d, J=4.5 Hz, 1H), 5.38-5.13 (m, 6H), 5.09-5.04 (m, 1H), 4.51 (d, J=6.5 Hz, 1H), 3.88-3.81 (m, 1H), 3.72-3.65 (m, 4H), 3.65-3.56 (m, 1H), 3.50-3.40 (m, 4H), 2.78-2.68 (m, 1H), 2.58 (dt, J=5.0, 12.7 Hz, 1H), 2.39 (dd, J=8.2, 13.4 Hz, 1H), 1.98 (br, dd, J=8.9, 13.4 Hz, 1H), 1.88-1.72 (m, 2H), 1.57 (s, 3H), 1.35 (s, 3H), 1.04-0.95 (m, 4H), 0.01 (s, 18H).

A solution of 64-12 (200 mg, 250 μmol) in AcOH (4 mL) and H₂O (2 mL) was stirred at 70° C. for 4 h. The mixture was filtered and concentrated under reduced pressure. The residue was purified by prep-HPLC (column: Boston Green ODS 150*30 mm*5 um; mobile phase: [water (0.225% FA)-ACN]; B %: 10%-40%, 7 min.) to give 64 (50 mg, 98% purity) as a white solid. ¹H NMR (400 MHz, CD₃OD) δ: 8.18 (s, 1H), 7.77 (s, 1H), 7.21 (s, 1H), 6.98-6.79 (m, 2H), 6.57 (d, J=4.5 Hz, 1H), 4.51 (dd, J=4.9, 8.9 Hz, 1H), 3.95 (d, J=5.0 Hz, 1H), 3.81-3.65 (m, 2H), 3.49 (d, J=9.3 Hz, 1H), 3.42 (s, 3H), 2.86-2.65 (m, 2H), 2.11 (dd, J=9.5, 13.6 Hz, 1H), 1.96-1.78 (m, 2H), 1.71 (dd, J=9.8, 13.3 Hz, 1H). ¹⁹F NMR (376 MHz, CD₃OD) δ: −125.839.

### Example 60

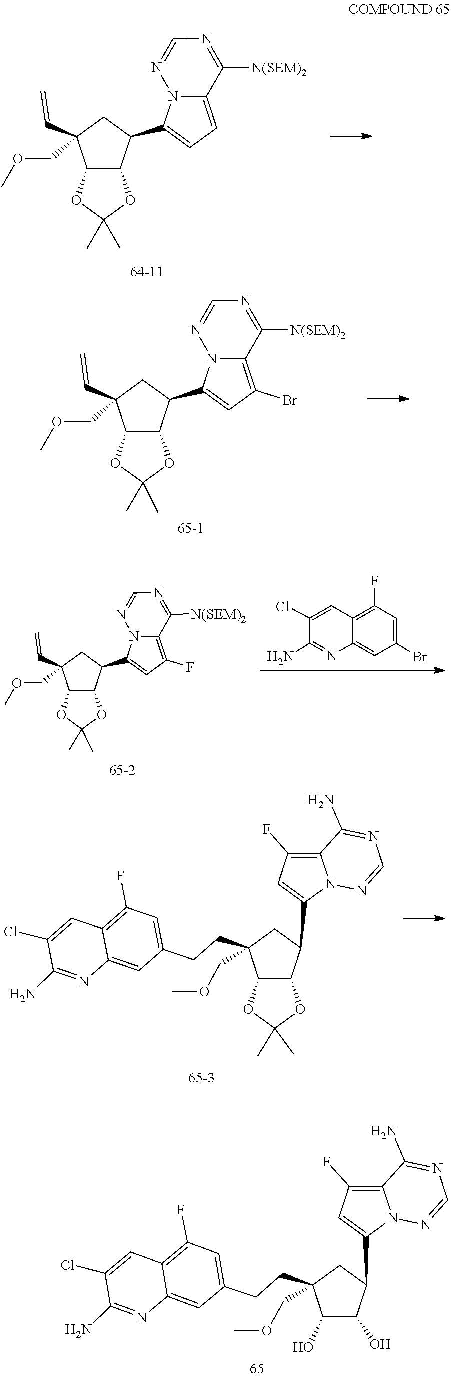

To a solution of 64-11 (2.02 g, 3.34 mmol) in DMF (20 mL) was added NBS (594 mg, 3.34 mmol) in one portion, and the mixture was stirred at 20° C. for 12 h. The mixture was diluted with water (30 mL) and extracted with EtOAc (2×30 mL). The combined organic layers were washed with brine (30 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜12% EA/PE gradient @ 30 mL/min) to give 65-1 (2.25 g, 96% purity) as a colorless oil. ¹H NMR (400 MHz, CDCl₃) δ: 8.05 (s, 1H), 6.68 (s, 1H), 5.96 (dd, J=10.8, 17.6 Hz, 1H), 5.22-5.01 (m, 6H), 4.96 (dd, J=4.8, 6.3 Hz, 1H), 4.65 (d, J=6.5 Hz, 1H), 3.86 (dt, J=4.6, 8.2 Hz, 1H), 3.61-3.52 (m, 2H), 3.52-3.44 (m, 4H), 3.41 (s, 3H), 2.50 (dd, J=8.2, 13.2 Hz, 1H), 2.14-2.07 (m, 1H), 1.57 (s, 3H), 1.34 (s, 3H), 0.93-0.83 (m, 4H), −0.05 (s, 18H).

To a solution of 65-1 (2.25 g, 3.29 mmol) in THF (75 mL) was added n-BuLi (2.5 M, 2.63 mL) dropwise at −78° C., and the mixture was stirred for 5 min. After adding NFSI (3.11 g, 9.87 mmol) at −78° C., the mixture was further stirred at −78° C. for 30 min. The reaction was quenched by the addition NH₄Cl (30 mL) at 0° C. and extracted with EtOAc (2×50 mL). The combined organic layers were washed with brine (50 mL), dried over Na₂SO₄, filtered and concentrated under reduced pressure to give a residue. The residue was purified by column chromatography (SiO₂, EA:MTBE=15:1 to 10:1) to give 65-2 (734 mg, 96% purity) as a yellow oil. LCMS: (ESI): m/z calcd. for: C₃₀H₅₂FN₄O₅Si₂ 623.34 [M+H]⁺, found 623.7. ¹H NMR (400 MHz, CDCl₃) δ: 7.90 (s, 1H), 6.30 (s, 1H), 5.93 (dd, J=10.9, 17.7 Hz, 1H), 5.18 (s, 4H), 5.14-5.03 (m, 2H), 4.93 (dd, J=4.4, 6.4 Hz, 1H), 4.65 (d, J=6.5 Hz, 1H), 3.85 (dt, J=4.1, 8.2 Hz, 1H), 3.62-3.49 (m, 6H), 3.40 (s, 3H), 2.48 (dd, J=8.3, 13.3 Hz, 1H), 2.12-2.00 (m, 1H), 1.57 (s, 3H), 1.35 (s, 3H), 0.97-0.91 (m, 4H), 0.03-−0.08 (m, 1H), −0.01 (s, 18H). ¹⁹F NMR (376 MHz, CDCl₃) δ: −148.446 (s, 1F).

To a solution of 65-2 (730 mg, 1.17 mmol) in THF (10 mL) was added 9-BBN dimer (709 mg, 2.93 mmol), and the mixture was stirred at 50° C. for 1.5 h under Ar atmosphere. The mixture was cooled to 20° C. A solution of K₃PO₄ (1.24 g, 5.86 mmol) in H₂O (2 mL) was added to the mixture, and the mixture was stirred for 0.5 h. After adding 7-bromo-3-chloro-5-fluoroquinolin-2-amine (420 mg, 1.52 mmol) and Pd(dppf)Cl₂ (171.50 mg, 234.38 μmol, 0.2 eq.) to the mixture, the mixture was degassed and purged with Ar (3×). The mixture was further stirred at 70° C. for 12 h. The mixture was then diluted with EtOAc (10 mL) and H₂O (10 mL), and extracted with EtOAc (2×20 mL). The combined organic layers were washed with brine (30 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜25% EA/PE gradient @ 30 mL/min) to give 65-3 (580 mg, 98% purity) as a yellow oil. LCMS: (ESI): m/z calcd. for: C₃₉H₅₈ClF₂N₆O₅Si₂ 819.36 [M+H]+, found 819.9. ¹H NMR (400 MHz, CDCl₃) δ: 8.16 (s, 1H), 7.88 (s, 1H), 7.21 (s, 1H), 6.77 (d, J=10.8 Hz, 1H), 6.33 (s, 1H), 5.36 (br s, 2H), 5.18 (s, 4H), 5.03-4.92 (m, 1H), 4.51 (d, J=6.8 Hz, 1H), 3.86-3.75 (m, 1H), 3.63-3.52 (m, 5H), 3.48 (d, J=9.8 Hz, 1H), 3.44 (s, 3H), 2.83-2.61 (m, 2H), 2.42 (dd, J=8.0, 13.3 Hz, 1H), 1.90-1.84 (m, 2H), 1.75 (br dd, J=4.5, 12.5 Hz, 1H), 1.56 (s, 3H), 1.34 (s, 3H), 0.99-0.89 (m, 4H), −0.02 (s, 18H). ¹⁹F NMR (376 MHz, CDCl₃) δ: −123.666 (s, 1F), −148.362 (s, 1F).

A solution of 65-3 (420 mg, 512 μmol) in CH₃COOH (8 mL) and H₂O (4 mL) was stirred at 70° C. for 4 h. The mixture was cooled to rt, neutralized with Na₂CO₃ at 0° C. and extracted with 2-Methyltetrahydrofuran (3×30 mL). The combined organic layers were washed with Na₂CO₃ (2×30 mL), dried over Na₂SO₄, filtered and concentrated under reduced pressure to give a crude product (266 mg), which was combined with other batches of crude product (26 mg and 126 mg) and purified by column chromatography (SiO₂, DCM/MeOH=30/l to 20/1) to give an impure product (280 mg). A second purification by SFC (column: Phenomenex-Cellulose-2 (250 mm*30 mm, 10 um); mobile phase: [0.1% NH₃H₂O ETOH]; B %: 55%-55%) provided 65 (168 mg, 99% purity) as a white solid. ¹H NMR (400 MHz, CD₃OD) δ: 8.18 (s, 1H), 7.66 (s, 1H), 7.21 (s, 1H), 6.86 (d, J=11.0 Hz, 1H), 6.35 (s, 1H), 4.46 (dd, J=5.0, 9.0 Hz, 1H), 3.92 (d, J=4.8 Hz, 1H), 3.79-3.63 (m, 2H), 3.47 (d, J=9.3 Hz, 1H), 3.41 (s, 3H), 2.86-2.64 (m, 2H), 2.07 (dd, J=9.5, 13.6 Hz, 1H), 1.94-1.77 (m, 2H), 1.65 (dd, J=10.0, 13.6 Hz, 1H). ¹⁹F NMR (376 MHz, CD₃OD) δ: −125.877 (s, 1F), −161.311 (s, 1F).

### Example 61

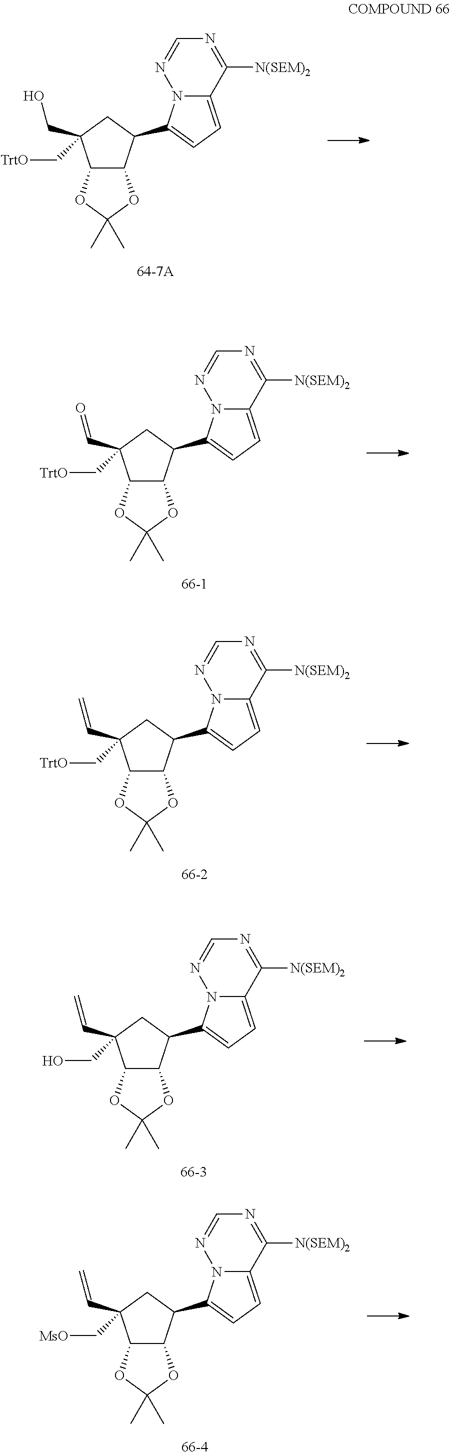

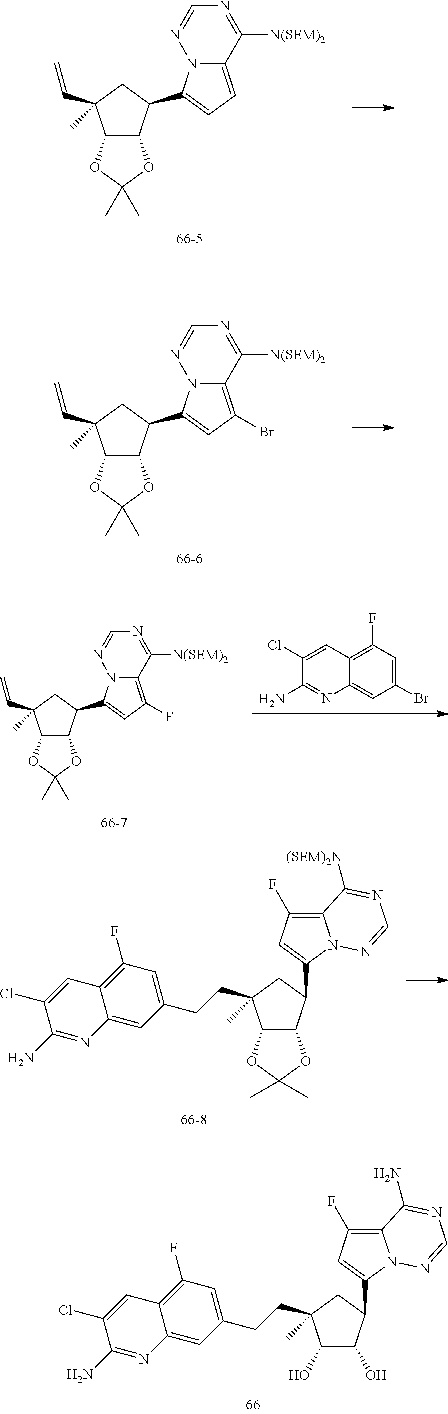

To a solution of 64-7A (532 mg, 635 μmol) in MeCN (10 mL) was added IBX (356 mg, 1.27 mmol), and the mixture was stirred at 65° C. for 2 h. The mixture was filtered and concentrated under reduced pressure to give a residue. The residue was purified by flash silica gel chromatography (ISCO®; 12 g SepaFlash® Silica Flash Column, Eluent of 0˜12% EA/PE gradient @ 20 mL/min) to give 66-1 (520 mg, 95% purity) as a white solid. LCMS: (ESI): m/z calcd. for C₄₇H₆₃N₄O₆Si₂ 835.42 [M+H]⁺, found 835.5. ¹H NMR (400 MHz, CDCl₃) δ: 9.33 (s, 1H), 7.97 (s, 1H), 7.44-7.36 (m, 7H), 7.32-7.28 (m, 2H), 7.26-7.16 (m, 6H), 6.91 (br d, J=4.3 Hz, 1H), 6.31 (d, J=4.6 Hz, 1H), 5.24-5.10 (m, 5H), 5.00 (dd, J=2.3, 5.8 Hz, 1H), 3.74-3.60 (m, 6H), 3.24 (d, J=9.1 Hz, 1H), 2.31 (d, J=6.4 Hz, 2H), 1.33 (s, 6H), 1.01-0.95 (m, 4H), 0.00 (s, 18H).

To a solution of MePPh₃Br (545 mg, 1.53 mmol) in THF (6 mL) was added t-BuOK (137 mg, 1.22 mmol) at 0° C., and the mixture was stirred at 0° C. for 1 h. A solution of 66-1 (510 mg, 610 μmol) in THF (3 mL) was added dropwise at 0° C., and the mixture was stirred at 20° C. for 12 h. The reaction was quenched by NH₄Cl (sat., aq., 30 mL) at 0° C., and extracted with EtOAc (2×20 mL). The combined organic layers were washed with brine (50 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜12% EA/PE gradient @ 30 mL/min) to give 66-2 (461 mg, 94% purity) as a white solid. LCMS: (ESI): m/z calcd. for C₄₈H₆₅N₄O₅Si₂ 833.44 [M+H]⁺, found 833.6. ¹H NMR (400 MHz, CDCl₃) δ: 7.97 (s, 1H), 7.50 (d, J=7.3 Hz, 6H), 7.32-7.28 (m, 6H), 7.26-7.21 (m, 3H), 6.96 (br s, 1H), 6.52 (d, J=4.5 Hz, 1H), 6.10 (dd, J=10.9, 17.4 Hz, 1H), 5.23 (s, 4H), 5.16-5.11 (m, 2H), 4.97 (dd, J=4.8, 6.8 Hz, 1H), 4.67-4.63 (m, 1H), 3.73-3.65 (m, 4H), 3.54 (dt, J=4.4, 8.5 Hz, 1H), 3.32 (d, J=9.0 Hz, 1H), 3.21-3.14 (m, 1H), 2.67 (dd, J=7.7, 13.2 Hz, 1H), 2.13 (br dd, J=9.8, 12.8 Hz, 1H), 1.25 (s, s, 6H), 1.04-0.96 (m, 4H), 0.03 (s, 18H).

To a solution of 66-2 (450 mg, 540 μmol) in DCM (9 mL) were added Et₃SiH (863 μL 5.40 mmol) and TFA (160 μL, 2.16 mmol) at 0° C., and the mixture was stirred at 0° C. for 2 h. The reaction was quenched by NaHCO₃ (sat., aq., 20 mL) at 0° C., and extracted with DCM (3×20 mL). The combined organic layers were washed with brine (30 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜20% EA/PE gradient @ 25 mL/min) to give 66-3 (295 mg, 97% purity) as a colorless oil. LCMS: (ESI): m/z calcd. for C₂₉H₅₁N₄O₅Si₂ 591.33 [M+H]⁺, found 591.8. ¹H NMR (400 MHz, CDCl₃) δ: 8.02-7.98 (m, 1H), 6.99 (br d, J=3.8 Hz, 1H), 6.56 (d, J=4.5 Hz, 1H), 5.97 (dd, J=10.8, 17.6 Hz, 1H), 5.23 (s, 4H), 5.18-5.10 (m, 2H), 5.06 (dd, J=4.8, 6.5 Hz, 1H), 4.78 (d, J=6.5 Hz, 1H), 3.93 (dq, J=4.8, 8.2 Hz, 2H), 3.74-3.63 (m, 5H), 2.58 (br d, J=5.5 Hz, 1H), 2.41 (dd, J=8.0, 13.3 Hz, 1H), 2.10 (dd, J=9.0, 13.3 Hz, 1H), 1.61 (s, 3H), 1.37 (s, 3H), 1.03-0.96 (m, 4H), 0.06-0.02 (m, 18H).

To a solution of 66-3 (1.49 g, 2.52 mmol) in DCM (15 mL) were added TEA (1.75 mL, 12.6 mmol) and MsCl (675.68 μL, 8.73 mmol) at 0° C., and the mixture was stirred at 20° C. for 1 h. The reaction was quenched by NaHCO₃ (sat., aq., 100 mL) at 0° C., and extracted with EA (2×40 mL). The combined organic layers were washed with brine (100 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜20% EA/PE gradient @35 mL/min) to give 66-5 (1.5 g, 95% purity) as a colorless oil. LCMS: (ESI): m/z calcd. for C₃₀H₅₃N₄O₇SSi₂ 669.31 [M+H]⁺, found 669.7. ¹H NMR (400 MHz, CDCl₃) δ: 7.99 (s, 1H), 6.94 (d, J=4.6 Hz, 1H), 6.54 (d, J=4.6 Hz, 1H), 5.85 (dd, J=10.9, 17.6 Hz, 1H), 5.22 (s, 4H), 5.20-5.05 (m, 3H), 4.71 (d, J=6.4 Hz, 1H), 4.43-4.34 (m, 2H), 3.90 (dt, J=3.9, 7.9 Hz, 1H), 3.72-3.63 (m, 4H), 3.04 (s, 3H), 2.47 (dd, J=8.2, 13.5 Hz, 1H), 2.22 (dd, J=7.9, 13.5 Hz, 1H), 1.56 (s, 3H), 1.35 (s, 3H), 1.05-0.94 (m, 4H), 0.02 (s, 18H).

To a solution of 66-4 (500 mg, 747 μmol) in PEG-400 (20 mL) was added NaBH₄ (283 mg, 7.47 mmol), and the mixture was stirred at 70° C. for 8 h under N₂. The mixture was cooled to rt, poured into water (30 mL) and extracted with EtOAc (3×30 mL). The combined organic phase was washed with brine (30 mL), dried over Na₂SO₄, filtered and concentrated to give a residue. The residue was purified by silica gel column (PE:EtOAc=100:0 to 20:1), and provide 66-5 (600 mg). LCMS: (ESI): m/z calcd. for C₂₉H₅₁N₄O₄Si₂ 575.3 [M+H]⁺, found 575.2. ¹H NMR (400 MHz, MeOD) δ: 7.93 (s, 1H), 7.02 (d, J=4.8 Hz, 1H), 6.67 (d, J=4.6 Hz, 1H), 5.91 (dd, J=10.8, 17.5 Hz, 1H), 5.23 (s, 4H), 5.03-4.91 (m, 3H), 4.55 (d, J=6.9 Hz, 1H), 3.88 (dt, J=4.7, 8.7 Hz, 1H), 3.71 (t, J=8.1 Hz, 4H), 2.26-1.99 (m, 2H), 1.52 (s, 3H), 1.32 (s, 3H), 1.21 (s, 3H), 1.01-0.93 (m, 4H), 0.00 (s, 18H).

To a solution of 66-5 (1.17 g, 2.04 mmol) in DMF (20 mL) was added NBS (362 mg, 2.04 mmol), and the mixture was stirred at 10° C. for 12 h. The mixture was diluted with water (10 mL) and extracted with EtOAc (3×20 mL). The combined organic layers were washed with brine (10 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜4% EA/PE gradient @ 20 mL/min) to afford 66-6 (1.25 g, 92% purity) as a light-yellow oil. LCMS: (ESI): m/z calcd. for C₂₉H₅₀BrN₄O₂Si₂ 655.25 [M+2+H]⁺, found 654.9. ¹H NMR (400 MHz, CDCl₃) δ: 8.05 (s, 1H), 6.70 (s, 1H), 5.92 (dd, J=10.7, 17.4 Hz, 1H), 5.14 (s, 4H), 5.09-4.88 (m, 3H), 4.53 (d, J=7.3 Hz, 1H), 3.89 (ddd, J=5.0, 7.6, 10.2 Hz, 1H), 3.52-3.44 (m, 4H), 2.25-2.04 (m, 2H), 1.58 (s, 3H), 1.34 (s, 3H), 1.26 (s, 3H), 0.92-0.85 (m, 4H), −0.04 (s, 18H).

To a solution of 66-6 (3.38 g, 5.17 mmol) in THF (30 mL) was added n-BuLi (662 mg, 10.3 mmol) dropwise at −78° C., and the mixture was stirred at −78° C. for 5 min. After adding NFSI (4.89 g, 15.5 mmol) in one portion to the mixture at −78° C., the mixture was further stirred at −78° C. for 30 min. The reaction was quenched by NH₄Cl (30 mL) and extracted with EtOAc (3×40 mL). The combined organic layers were washed with brine (30 mL), dried over Na₂SO₄, filtered and concentrated under reduced pressure to give a residue. The residue was purified by column chromatography (SiO₂, PE:MTBE=30:1 to 25:1) to afford 66-7 (515 mg, 99% purity) as a light-yellow oil. LCMS: (ESI): m/z calcd. for C₂₉H₅₀FN₄O₄Si₂ 593.33 [M+H]⁺, found 593.2. ¹H NMR (400 MHz, CDCl₃) δ: 7.89 (s, 1H), 6.32 (s, 1H), 5.91 (dd, J=10.8, 17.3 Hz, 1H), 5.18 (s, 4H), 5.06-4.97 (m, 2H), 4.93-4.88 (m, 1H), 4.52 (d, J=7.0 Hz, 1H), 3.89 (ddd, J=4.8, 7.8, 9.8 Hz, 1H), 3.60-3.53 (m, 4H), 2.22-2.08 (m, 2H), 1.57 (s, 3H), 1.34 (s, 3H), 1.25 (s, 3H), 0.97-0.91 (m, 4H), 0.00-0.02 (m, 18H). ¹⁹F NMR (400 MHz, CDCl₃) δ: 148.38.

To a solution of 66-7 (200 mg, 337 μmol) in THF (5 mL) was added 9-BBN dimer (204 mg, 843 μmol). The mixture was stirred at 50° C. for 2.5 h under Ar atmosphere and cooled to 20° C. After adding 7-bromo-3-chloro-5-fluoroquinolin-2-amine (120.82 mg, 438 μmol, 1.3 eq.) and Pd(dppf)Cl2 (49 mg, 67 μmol) into the mixture, the mixture was degassed with Ar (3×) and stirred at 70° C. for 12 h. The mixture was diluted with water (10 mL) and extracted with EtOAc (3×10 mL). The combined organic layers were washed with brine (20 mL), dried over Na₂SO₄, filtered and concentrated under reduced pressure to give a residue. The residue was purified by flash silica gel chromatography (ISCO®; 12 g SepaFlash® Silica Flash Column, Eluent of 5˜25% EA/PE gradient @ 25 mL/min) to give 66-8 (181 mg, 92% purity) as a white solid. LCMS: (ESI): m/z calcd. for: C₃₈H₅₆ClF₂N₆O₄Si₂ 789.35 [M+H]⁺, found 789.2. ¹H NMR (400 MHz, CDCl₃) δ: 8.17 (s, 1H), 7.88 (s, 1H), 7.24 (s, 1H), 6.79 (d, J=10.8 Hz, 1H), 6.34 (s, 1H), 5.44 (br s, 2H), 5.18 (s, 4H), 4.94 (t, J=6.3 Hz, 1H), 4.43 (d, J=7.3 Hz, 1H), 3.94-3.78 (m, 1H), 3.63-3.49 (m, 4H), 2.85-2.61 (m, 2H), 2.17 (br dd, J=7.3, 13.1 Hz, 1H), 1.86 (br s, 2H), 1.75 (br s, 1H), 1.58 (s, 3H), 1.35 (s, 3H), 1.23 (s, 3H), 0.98-0.88 (m, 4H), −0.02 (s, 18H). ¹⁹F NMR (376 MHz, CDCl₃) δ: −123.46, −148.37.

A mixture of 66-8 (406 mg, 514 μmol) in AcOH (4.6 mL) and H₂O (2.4 mL) was stirred at 70° C. for 9 h. The mixture was cooled to rt, basified by salt Na₂CO₃ at 0° C. and extracted with 2-Me-THF (3×20 mL). The combined organic layers were washed with Na₂CO₃ (2×30 mL), dried over Na₂SO₄, filtered and concentrated under reduced pressure to give a crude product (251 mg). The crude was combined with another batch (22 mg, crude), and purified by prep-HPLC (column: Phenomenex Gemini-NX 80*40 mm*3 um; mobile phase: [water (0.05% NH₃.H₂O)-ACN]; B %: 35%-65%, 8 min) to give 66. LCMS: (ESI): m/z calcd. For: C₂₃H₂₄ClF₂N₆O₂ 489.15 [M+H]⁺, found 489.1.

### Example 62

### Description of LCMS Conditions

| | | | | | | Run |
|---|---|---|---|---|---|---|
| Method | | | | | Flow | time |
| Code | Instrument | Column | Mobile Phase | Gradient | mL/min | (min) |
| 1 | A | Xbridge | A: water(4 L) + | 0%-60% (solvent B) | 0.8 | 7 |
| | | Shield RP- | NH₃•H₂O(0.8 mL) | over 6 minutes and | | |
| | | 18, 5 um, | B: acetonitrile | holding at 60% for | | |
| | | 2.1 * 50 mm | | 0.5 minutes | | |
| 2 | A | Xtimate | A: water(4 L) + | 0%-30% (solvent B) | 0.8 | 4 |
| | | C18 | TFA(1.5 mL) | over 3 minutes and | | |
| | | 2.1 * 30 mm, | B: acetonitrile(4L) + | holding at 30% for | | |
| | | 3 um | TFA(0.75 mL) | 0.5 minutes | | |
| 3 | B | Xtimate | A: water(4L) + | 0%-60% (solvent B) | 1.2 | 2 |
| | | C18 | TFA(1.5 mL) | over 0.9 minutes and | | |
| | | 2.1 * 30 mm, | B: acetonitrile(4L) + | holding at 60% for | | |
| | | 3um | TFA(0.75 mL) | 0.6 minutes | | |
| 4 | A | Xtimate | A: water(4 L) + | 0%-30% (solvent B) | 1.2 | 2 |
| | | C18 | TFA(1.5 mL) | over 0.9 minutes and | | |
| | | 2.1 * 30 mm, | B: acetonitrile | holding at 30% | | |
| | | 3 um | | for 0.6 minutes | | |
| 5 | A | Xbridge | A: water(4 L) + | 0%-30% (solvent B) | 1 | 3 |
| | | Shield RP- | NH₃•H₂O(0.8 mL) | over 2 minutes and | | |
| | | 18, 5 um, | B: acetonitrile | holding at 30% | | |
| | | 2.1 * 50 mm | | for 0.48 minutes | | |
| 6 | A | Xtimate | A: water(4 L) + | 0%-60% (solvent B) | 0.8 | 7 |
| | | C18 | TFA(1.5 mL) | over 6 minutes and | | |
| | | 2.1 * 30 mm, | B: acetonitrile(4 L) + | holding at 60% for | | |
| | | 3 um | TFA(0.75 mL) | 0.5 minutes | | |
| 7 | A | Xbridge | A: water(4 L) + | 10%-80% (solvent B) | 0.8 | 7 |
| | | Shield RP- | NH₃•H₂O(0.8 mL) | over 6 minutes | | |
| | | 18, 5 um, | B: acetonitrile | and holding at 80% | | |
| | | 2.1 * 50 mm | | for 0.5 minutes | | |
| 8 | A | Xbridge | A: water(4 L) + | 10%-80% (solvent B) | 1 | 3 |
| | | Shield RP- | NH₃•H₂O(0.8 mL) | over 2 minutes | | |
| | | 18, 5 um, | B: acetonitrile | and holding at 80% | | |
| | | 2.1 * 50 mm | | for 0.48 minutes | | |
| 9 | A | Xtimate | A:water(4 L) + | 0%-30% (solvent B) | 0.8 | 7 |
| | | C18 | TFA(1.5 mL) | over 6 minutes and | | |
| | | 2.1 * 30 mm, | B: acetonitrile(4 L) + | holding at 30% for | | |
| | | 3um | TFA(0.75 mL) | 0.5 minutes | | |
| 10 | A | Xbridge | A: water(4 L) + | 0%-60% (solvent B) | 1 | 3 |
| | | Shield RP- | NH₃•H₂O(0.8 mL) | over 2 minutes and | | |
| | | 18, 5 um, | B: acetonitrile | holding at 60% | | |
| | | 2.1 * 50 mm | | for 0.48 minutes | | |
| 11 | A | Xtimate | A: water(4 L) + | 0%-60% (solvent B) | 0.8 | 7 |
| | | C18 | TFA(1.5 mL) | over 6 minutes and | | |
| | | 2.1 * 30 mm, | B: acetonitrile | holding at 60% for | | |
| | | 3um | | 0.5 minutes | | |
| 12 | A | Xtimate | A: water(4 L) + | 0%-30% (solvent B) | 0.8 | 7 |
| | | C18 | TFA(1.5 mL) | over 6 minutes and | | |
| | | 2.1 * 30 mm, | B: acetonitrile | holding at 30% | | |
| | | 3um | | for 0.5minutes | | |
| 13 | A | Xtimate | A: water(4 L) + | 0%-60% (solvent B) | 0.8 | 4 |
| | | C18 | TFA(1.5 mL) | over 3 minutes and | | |
| | | 2.1 * 30 mm, | B: acetonitrile | holding at 60% for | | |
| | | 3 um | | 0.5 minutes | | |

A = SHIMADZU LC20-MS2020; B = Agilent LC1200-MS6110; Column Temp = 50° C.

| | Rt | | Method |
|---|---|---|---|
| Compound | (min) | [M + H]⁺ | code |
| 1 | 4.125 | 499.1 | 1 |
| 2 | 2 .663 | 517.2 | 2 |
| | | [M + Na]⁺ | |
| 3 | 1.177 | 516.1 | 3 |
| 4 | 1.299 | 495.2 | 4 |
| 5 | 2.546 | 419.2 | 5 |
| 6 | 2.498 | 419.2 | 5 |
| 7 | 4.160 | 421.0 | 1 |
| 8 | 4.209 | 421.1 | 1 |
| 9 | 4.051 | 503.1 | 1 |
| 10 | 4.114 | 433.3 | 1 |
| 11 | 4.364 | 487.1 | 1 |
| 12 | 2.604 | 419.3 | 7 |
| 13 | 3.681 | 417.2 | 1 |
| 14 | 4.448 | 459.2 | 1 |
| 15 | 1.548 | 497.1 | 8 |
| 16 | 3.260 | 407.2 | 1 |
| 17 | 2.022 | 499.1 | 1 |
| 18 | 4.048 | 453.3 | 9 |
| 19 | 4.187 | 451.2 | 1 |
| 20 | 2.040 | 533.5 | 8 |
| 20A | 4.163 | 533.3 | 7 |
| 21 | 2.596 | 436.2 | 6 |
| 21A | 2.610 | 436.3 | 6 |
| 22 | 1.705 | 418.2 | 10 |
| 23 | 2.045 | 469.2 | 10 |
| 24 | 3.333 | 420.3 | 1 |
| 25A | 4.064 | 557.3 | 7 |
| 26 | 4.241 | 559.5 | 7 |
| 26-A | 4.168 | 559.3 | 7 |
| 27 | 1.946 | 444.2 | 10 |
| 28 | 1.664 | 467.2 | 8 |
| 29 | 1.760 | 450.2 | 10 |
| 30 | 4.450 | 471.2 | 9 |
| 31 | 1.939 | 475.2 | 10 |
| 32 | 2.892 | 470.3 | 6 |
| 33 | 4.180 | 567.2 | 7 |
| 34 | 1.794 | 488.4 | 8 |
| 35 | 1.764 | 423.2 | 10 |
| 36 | 4.249 | 475.1 | 1 |
| 37 | 3.764 | 437.2 | 9 |
| 38 | 2.561 | 418.3 | 2 |
| 39 | 2.905 | 470.2 | 2 |
| 40 | 3.457 | 487.3 | 9 |
| 41 | 2.912 | 455.2 | 11 |
| 42 | 4.523 | 454.1 | 12 |
| 43 | 3.710 | 488.2 | 7 |
| 44 | 4.259 | 482.2 | 1 |
| 45 | 3.745 | 500.2 | 1 |
| 46 | 4.207 | 431.2 | 1 |
| 47 | 3.688 | 397.3 | 1 |
| 48 | 4.037 | 419.3 | 1 |
| 49 | 3.176 | 470.1 | 6 |
| 50 | 3.275 | 516.1 | 6 |
| 51 | 1.724 | 483.3 | 8 |
| 52 | 3.197 | 481.2 | 7 |
| 53 | 3.593 | 527.1* | 6 |
| 54 | 3.342 | 533.1** | 6 |
| 55 | 4.501 | 533.4 | 7 |
| 56 | 3.760 | 420.3 | 1 |
| 57 | 4.527 | 471.16 | 1 |
| 58 | 3.106 | 529.2 | 7 |
| 59 | 4.311 | 499.2** | 1 |
| 60 | 3.299 | 501.2 | 7 |
| 61 | 4.436 | 515.2 | 1 |
| 62 | 4.487 | 509.0 | 1 |
| 63 | 3.007 | 509.0** | 7 |
| 64 | 2.160 | 501.1 | 13 |
| 65 | 3.480 | 519.1 | 7 |
| 66 | 3.594 | 489.1 | 7 |

*[M + Na]⁺
**[M + 2 + H]⁺

### Example 63

### Additional Compounds

The foregoing syntheses are exemplary and can be used as a starting point to prepare a large number of additional compounds. Examples of compounds of Formula (I), and pharmaceutically acceptable salts thereof, that can be prepared in various ways, including those synthetic schemes shown and described herein, are provided below. Those skilled in the art will be able to recognize modifications of the disclosed syntheses and to devise routes based on the disclosures herein; all such modifications and alternate routes are within the scope of the claims.

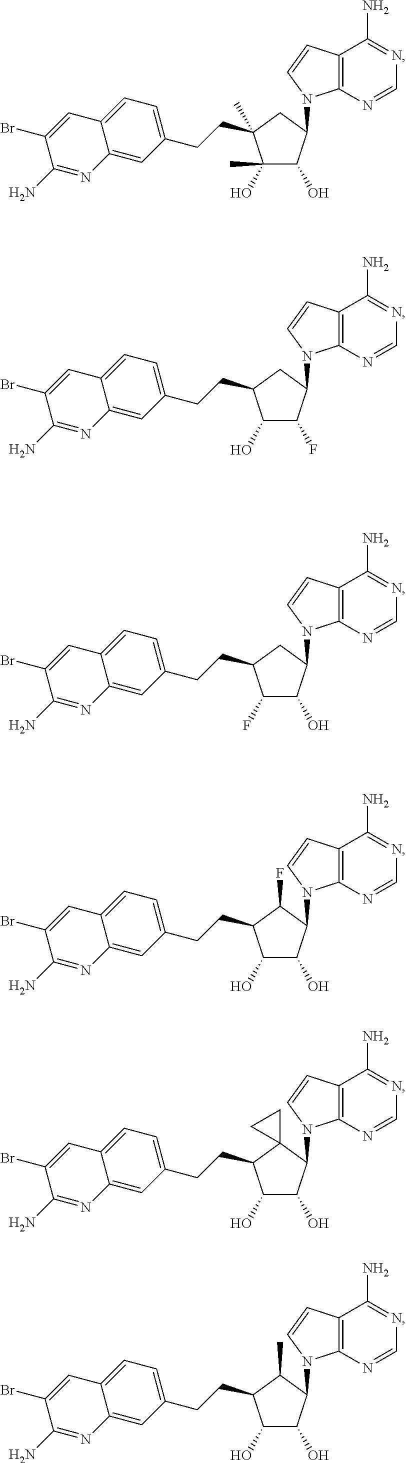 [SMILES: C[C@]1(CCc2ccc3cc(Br)c(N)nc3c2)C[C@@H](n2ccc3c(N)ncnc32)[C@H](O)[C@]1(C)O ; Nc1nc2cc(CC[C@H]3C[C@@H](n4ccc5c(N)ncnc54)[C@H](F)[C@@H]3O)ccc2cc1Br ; Nc1nc2cc(CC[C@H]3C[C@@H](n4ccc5c(N)ncnc54)[C@H](O)[C@@H]3F)ccc2cc1Br ; Nc1nc2cc(CC[C@@H]3[C@@H](O)[C@@H](O)[C@H](n4ccc5c(N)ncnc54)[C@@H]3F)ccc2cc1Br ; Nc1nc2cc(CC[C@@H]3[C@@H](O)[C@@H](O)[C@H](n4ccc5c(N)ncnc54)C34CC4)ccc2cc1Br ; C[C@@H]1[C@H](CCc2ccc3cc(Br)c(N)nc3c2)[C@@H](O)[C@@H](O)[C@@H]1n1ccc2c(N)ncnc21]

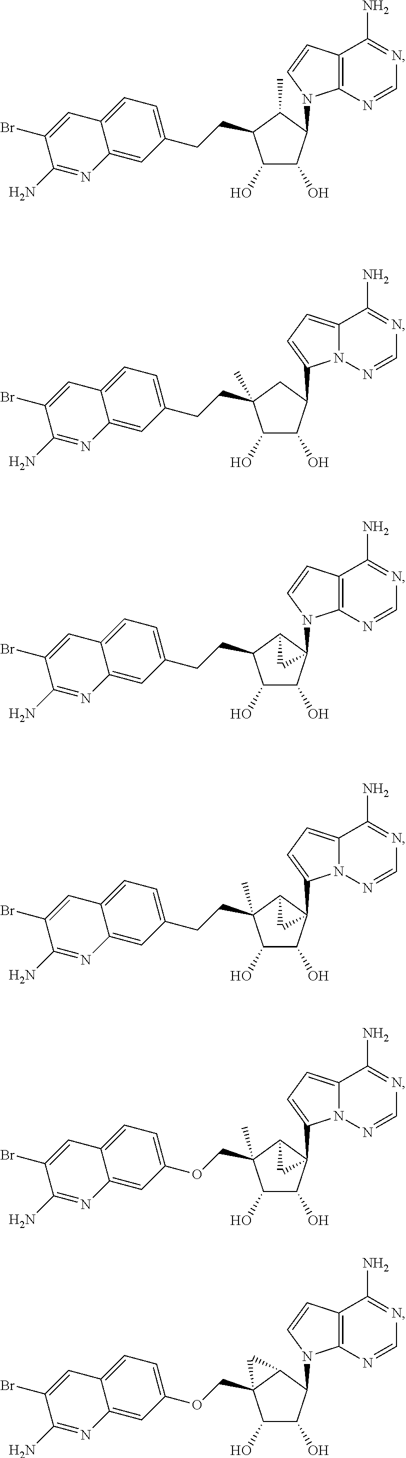 [SMILES: C[C@H]1[C@H](CCc2ccc3cc(Br)c(N)nc3c2)[C@@H](O)[C@@H](O)[C@@H]1n1ccc2c(N)ncnc21 ; C[C@]1(CCc2ccc3cc(Br)c(N)nc3c2)C[C@@H](c2ccc3c(N)ncnn23)[C@H](O)[C@@H]1O ; Nc1nc2cc(CC[C@@H]3[C@@H](O)[C@@H](O)[C@]4(n5ccc6c(N)ncnc65)C[C@@H]34)ccc2cc1Br ; C[C@@]1(CCc2ccc3cc(Br)c(N)nc3c2)[C@@H](O)[C@@H](O)[C@]2(c3ccc4c(N)ncnn34)C[C@@H]12 ; C[C@@]1(COc2ccc3cc(Br)c(N)nc3c2)[C@@H](O)[C@@H](O)[C@]2(c3ccc4c(N)ncnn34)C[C@@H]12 ; Nc1nc2cc(OC[C@@]34C[C@@H]3[C@@H](n3ccc5c(N)ncnc53)[C@H](O)[C@@H]4O)ccc2cc1Br]

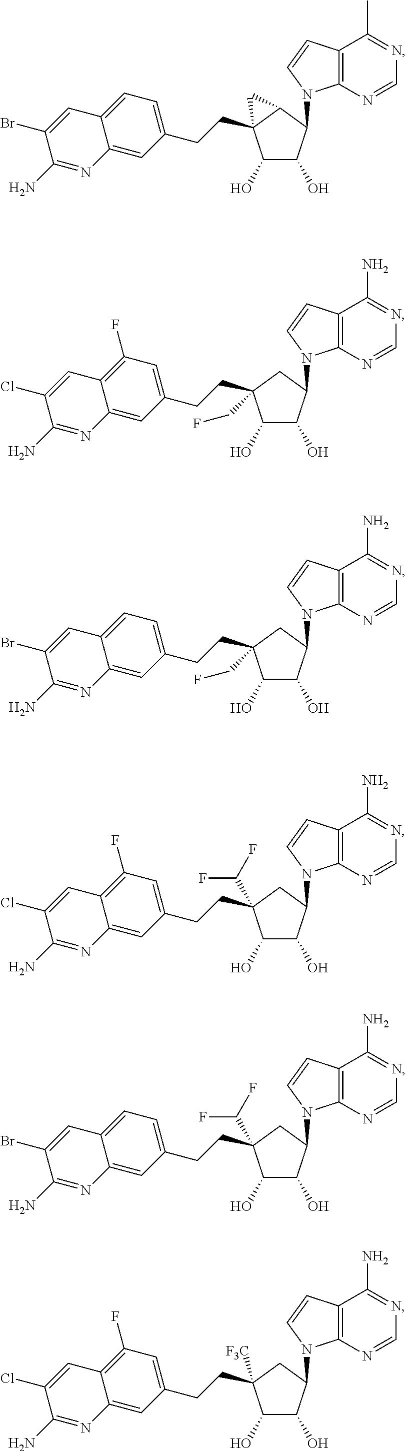 [SMILES: Cc1ncnc2c1ccn2[C@H]1[C@H](O)[C@H](O)[C@]2(CCc3ccc4cc(Br)c(N)nc4c3)C[C@H]12 ; Nc1nc2cc(CC[C@@]3(CF)C[C@@H](n4ccc5c(N)ncnc54)[C@H](O)[C@@H]3O)cc(F)c2cc1Cl ; Nc1nc2cc(CC[C@@]3(CF)C[C@@H](n4ccc5c(N)ncnc54)[C@H](O)[C@@H]3O)ccc2cc1Br ; Nc1nc2cc(CC[C@@]3(C(F)F)C[C@@H](n4ccc5c(N)ncnc54)[C@H](O)[C@@H]3O)cc(F)c2cc1Cl ; Nc1nc2cc(CC[C@@]3(C(F)F)C[C@@H](n4ccc5c(N)ncnc54)[C@H](O)[C@@H]3O)ccc2cc1Br ; Nc1nc2cc(CC[C@@]3(C(F)(F)F)C[C@@H](n4ccc5c(N)ncnc54)[C@H](O)[C@@H]3O)cc(F)c2cc1Cl]

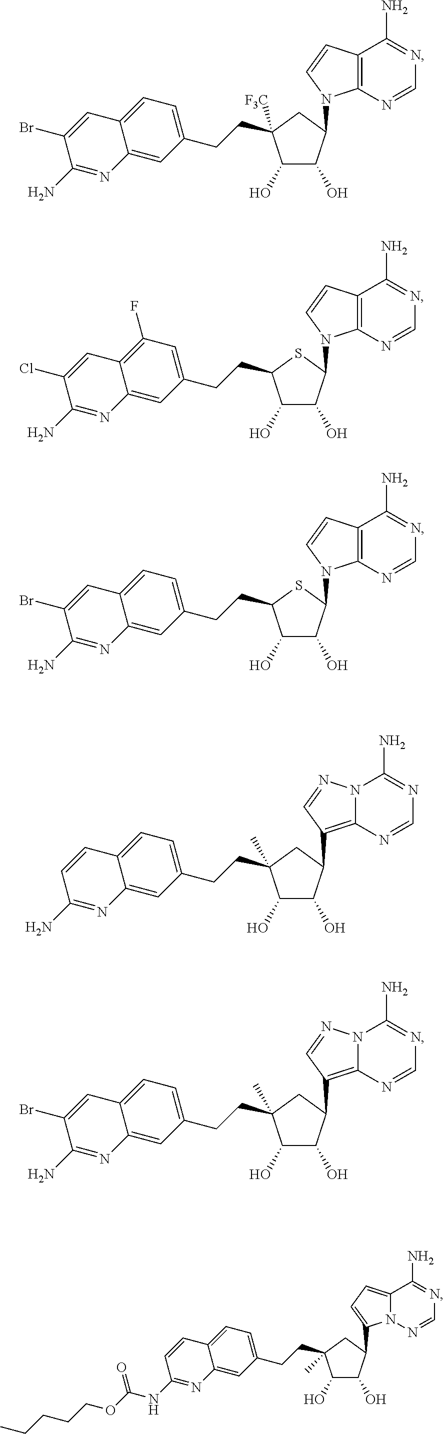 [SMILES: Nc1nc2cc(CC[C@@]3(C(F)(F)F)C[C@@H](n4ccc5c(N)ncnc54)[C@H](O)[C@@H]3O)ccc2cc1Br ; Nc1nc2cc(CC[C@H]3S[C@@H](n4ccc5c(N)ncnc54)[C@H](O)[C@@H]3O)cc(F)c2cc1Cl ; Nc1nc2cc(CC[C@H]3S[C@@H](n4ccc5c(N)ncnc54)[C@H](O)[C@@H]3O)ccc2cc1Br ; C[C@]1(CCc2ccc3ccc(N)nc3c2)C[C@@H](c2cnn3c(N)ncnc23)[C@H](O)[C@@H]1O ; C[C@]1(CCc2ccc3cc(Br)c(N)nc3c2)C[C@@H](c2cnn3c(N)ncnc23)[C@H](O)[C@@H]1O ; CCCCCOC(=O)Nc1ccc2ccc(CC[C@@]3(C)C[C@@H](c4ccc5c(N)ncnn45)[C@H](O)[C@@H]3O)cc2n1]

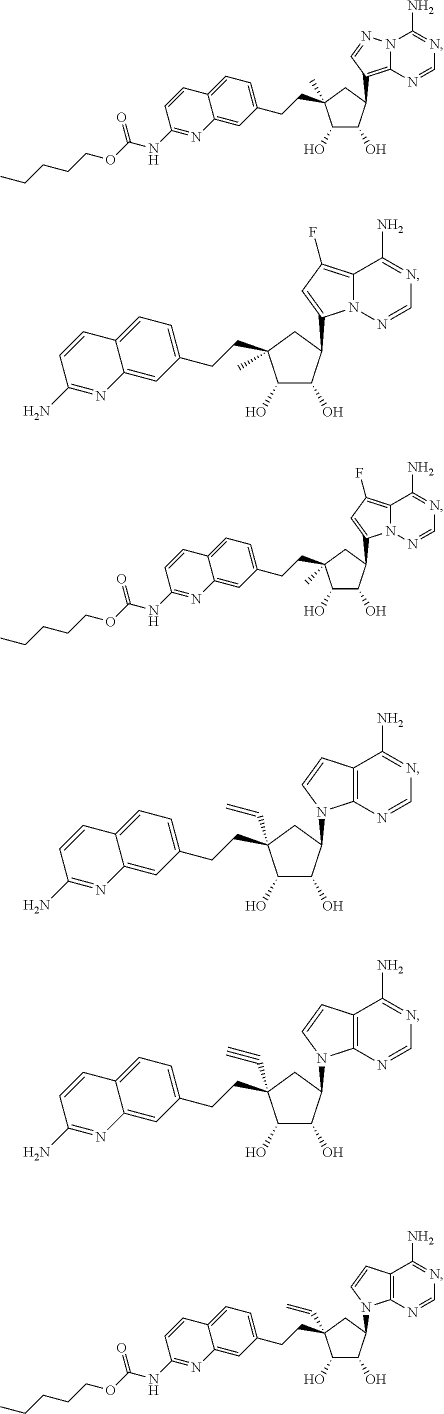 [SMILES: CCCCCOC(=O)Nc1ccc2ccc(CC[C@@]3(C)C[C@@H](c4cnn5c(N)ncnc45)[C@H](O)[C@@H]3O)cc2n1 ; C[C@]1(CCc2ccc3ccc(N)nc3c2)C[C@@H](c2cc(F)c3c(N)ncnn23)[C@H](O)[C@@H]1O ; CCCCCOC(=O)Nc1ccc2ccc(CC[C@@]3(C)C[C@@H](c4cc(F)c5c(N)ncnn45)[C@H](O)[C@@H]3O)cc2n1 ; C=C[C@]1(CCc2ccc3ccc(N)nc3c2)C[C@@H](n2ccc3c(N)ncnc32)[C@H](O)[C@@H]1O ; C#C[C@]1(CCc2ccc3ccc(N)nc3c2)C[C@@H](n2ccc3c(N)ncnc32)[C@H](O)[C@@H]1O ; C=C[C@]1(CCc2ccc3ccc(NC(=O)OCCCCC)nc3c2)C[C@@H](n2ccc3c(N)ncnc32)[C@H](O)[C@@H]1O]

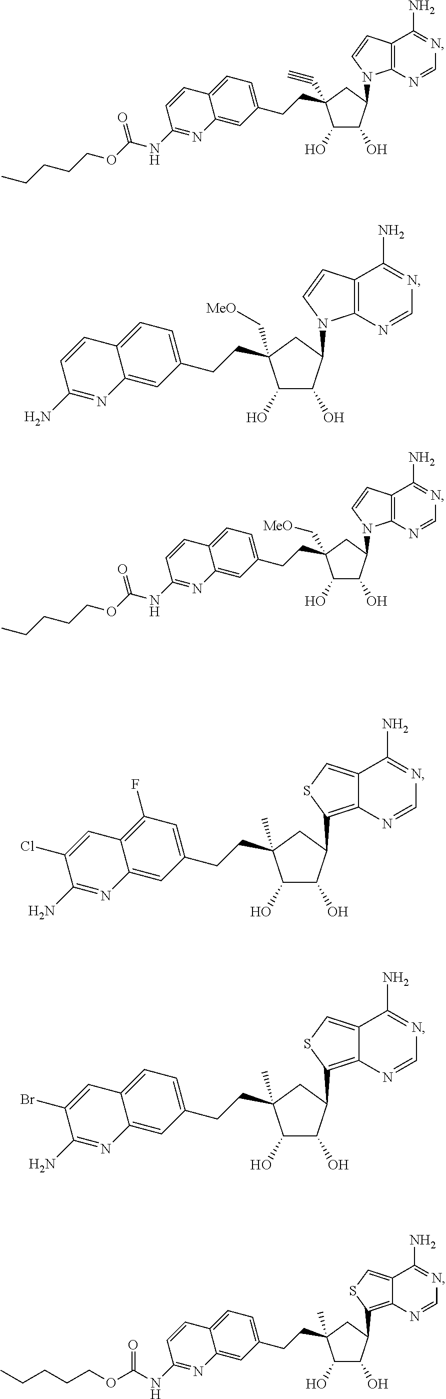 [SMILES: C#C[C@]1(CCc2ccc3ccc(NC(=O)OCCCCC)nc3c2)C[C@@H](n2ccc3c(N)ncnc32)[C@H](O)[C@@H]1O ; COC[C@]1(CCc2ccc3ccc(N)nc3c2)C[C@@H](n2ccc3c(N)ncnc32)[C@H](O)[C@@H]1O ; CCCCCOC(=O)Nc1ccc2ccc(CC[C@@]3(COC)C[C@@H](n4ccc5c(N)ncnc54)[C@H](O)[C@@H]3O)cc2n1 ; C[C@]1(CCc2cc(F)c3cc(Cl)c(N)nc3c2)C[C@@H](c2scc3c(N)ncnc23)[C@H](O)[C@@H]1O ; C[C@]1(CCc2ccc3cc(Br)c(N)nc3c2)C[C@@H](c2scc3c(N)ncnc23)[C@H](O)[C@@H]1O ; CCCCCOC(=O)Nc1ccc2ccc(CC[C@@]3(C)C[C@@H](c4scc5c(N)ncnc45)[C@H](O)[C@@H]3O)cc2n1]

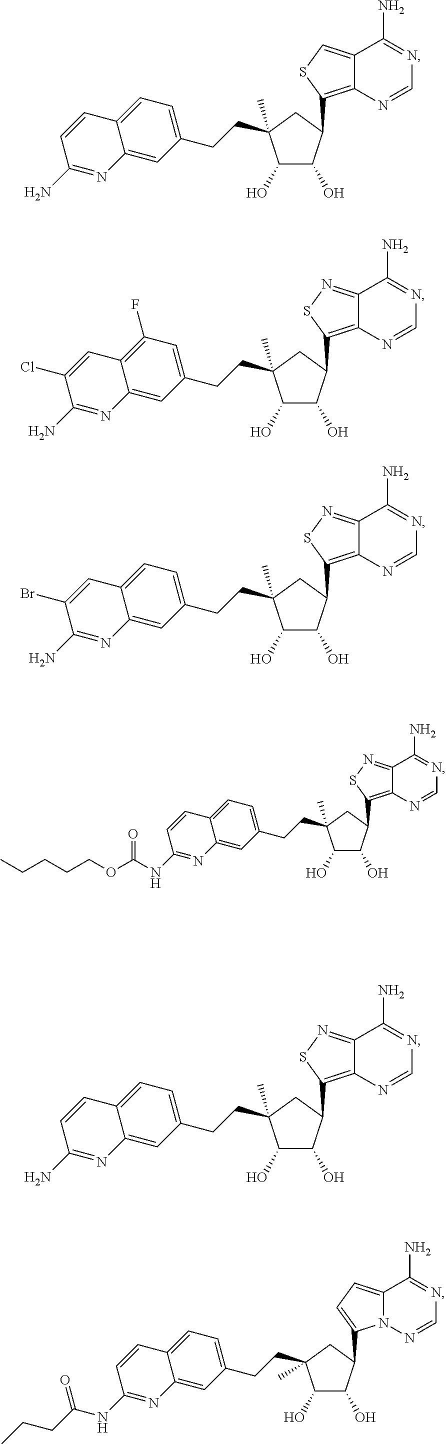 [SMILES: C[C@]1(CCc2ccc3ccc(N)nc3c2)C[C@@H](c2scc3c(N)ncnc23)[C@H](O)[C@@H]1O ; C[C@]1(CCc2cc(F)c3cc(Cl)c(N)nc3c2)C[C@@H](c2snc3c(N)ncnc23)[C@H](O)[C@@H]1O ; C[C@]1(CCc2ccc3cc(Br)c(N)nc3c2)C[C@@H](c2snc3c(N)ncnc23)[C@H](O)[C@@H]1O ; CCCCCOC(=O)Nc1ccc2ccc(CC[C@@]3(C)C[C@@H](c4snc5c(N)ncnc45)[C@H](O)[C@@H]3O)cc2n1 ; C[C@]1(CCc2ccc3ccc(N)nc3c2)C[C@@H](c2snc3c(N)ncnc23)[C@H](O)[C@@H]1O ; CCCC(=O)Nc1ccc2ccc(CC[C@@]3(C)C[C@@H](c4ccc5c(N)ncnn45)[C@H](O)[C@@H]3O)cc2n1]

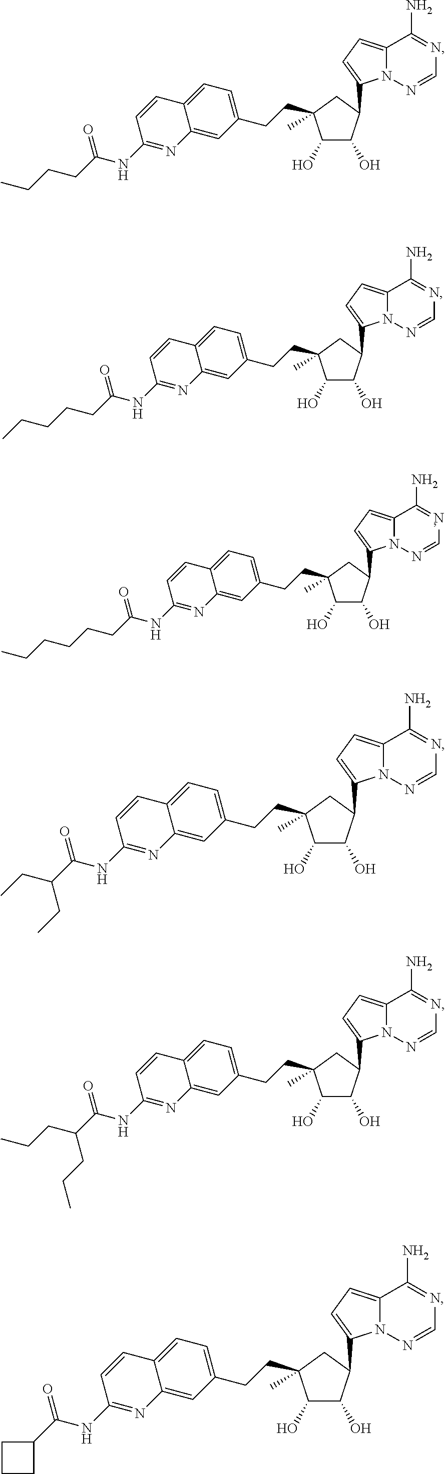 [SMILES: CCCCC(=O)Nc1ccc2ccc(CC[C@@]3(C)C[C@@H](c4ccc5c(N)ncnn45)[C@H](O)[C@@H]3O)cc2n1 ; CCCCCC(=O)Nc1ccc2ccc(CC[C@@]3(C)C[C@@H](c4ccc5c(N)ncnn45)[C@H](O)[C@@H]3O)cc2n1 ; CCCCCCC(=O)Nc1ccc2ccc(CC[C@@]3(C)C[C@@H](c4ccc5c(N)ncnn45)[C@H](O)[C@@H]3O)cc2n1 ; CCC(CC)C(=O)Nc1ccc2ccc(CC[C@@]3(C)C[C@@H](c4ccc5c(N)ncnn45)[C@H](O)[C@@H]3O)cc2n1 ; CCCC(CCC)C(=O)Nc1ccc2ccc(CC[C@@]3(C)C[C@@H](c4ccc5c(N)ncnn45)[C@H](O)[C@@H]3O)cc2n1 ; C[C@]1(CCc2ccc3ccc(NC(=O)C4CCC4)nc3c2)C[C@@H](c2ccc3c(N)ncnn23)[C@H](O)[C@@H]1O]

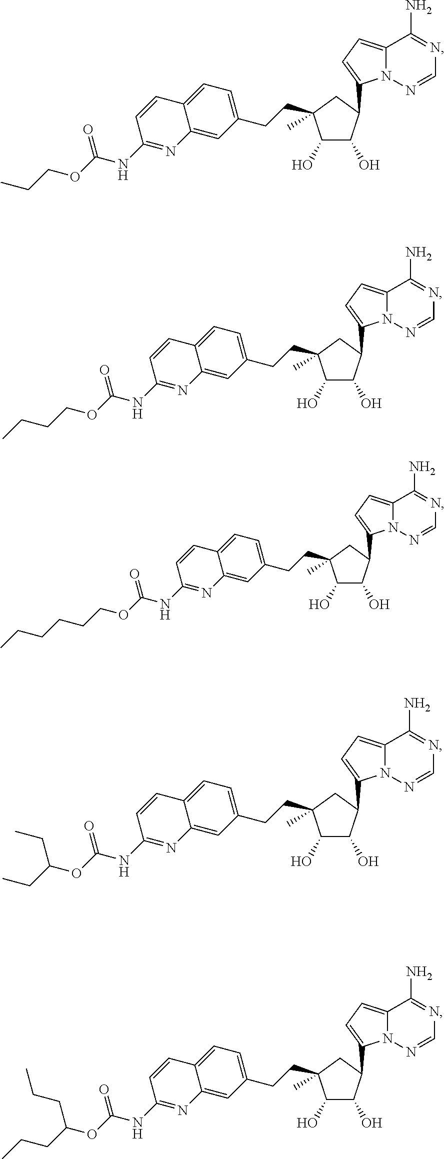 [SMILES: CCCOC(=O)Nc1ccc2ccc(CC[C@@]3(C)C[C@@H](c4ccc5c(N)ncnn45)[C@H](O)[C@@H]3O)cc2n1 ; CCCCOC(=O)Nc1ccc2ccc(CC[C@@]3(C)C[C@@H](c4ccc5c(N)ncnn45)[C@H](O)[C@@H]3O)cc2n1 ; CCCCCCOC(=O)Nc1ccc2ccc(CC[C@@]3(C)C[C@@H](c4ccc5c(N)ncnn45)[C@H](O)[C@@H]3O)cc2n1 ; CCC(CC)OC(=O)Nc1ccc2ccc(CC[C@@]3(C)C[C@@H](c4ccc5c(N)ncnn45)[C@H](O)[C@@H]3O)cc2n1 ; CCCC(CCC)OC(=O)Nc1ccc2ccc(CC[C@@]3(C)C[C@@H](c4ccc5c(N)ncnn45)[C@H](O)[C@@H]3O)cc2n1]

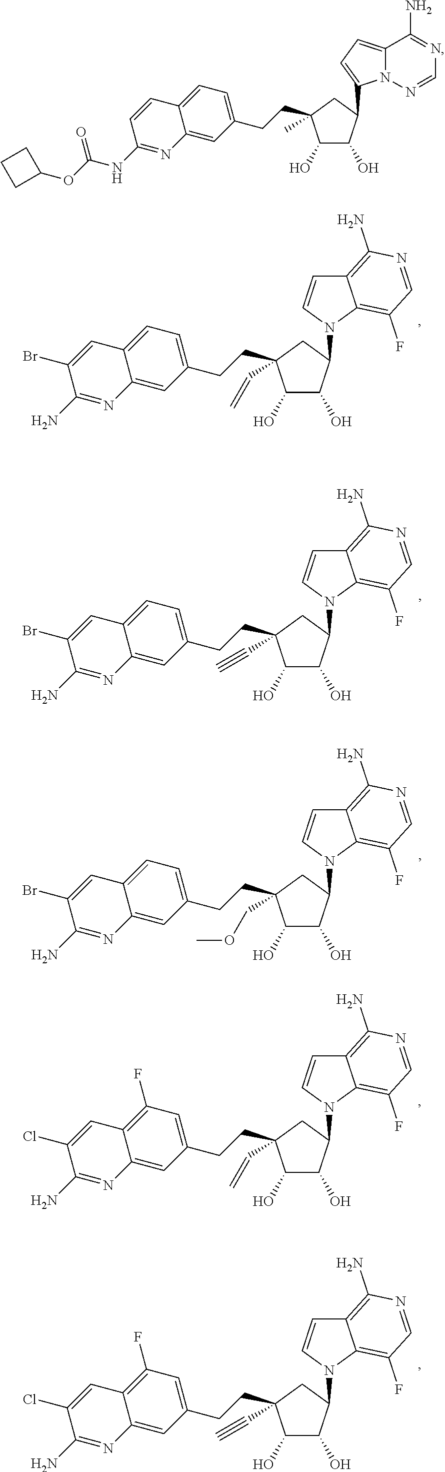 [SMILES: C[C@]1(CCc2ccc3ccc(NC(=O)OC4CCC4)nc3c2)C[C@@H](c2ccc3c(N)ncnn23)[C@H](O)[C@@H]1O ; C=C[C@]1(CCc2ccc3cc(Br)c(N)nc3c2)C[C@@H](n2ccc3c(N)ncc(F)c32)[C@H](O)[C@@H]1O ; C#C[C@]1(CCc2ccc3cc(Br)c(N)nc3c2)C[C@@H](n2ccc3c(N)ncc(F)c32)[C@H](O)[C@@H]1O ; COC[C@]1(CCc2ccc3cc(Br)c(N)nc3c2)C[C@@H](n2ccc3c(N)ncc(F)c32)[C@H](O)[C@@H]1O ; C=C[C@]1(CCc2cc(F)c3cc(Cl)c(N)nc3c2)C[C@@H](n2ccc3c(N)ncc(F)c32)[C@H](O)[C@@H]1O ; C#C[C@]1(CCc2cc(F)c3cc(Cl)c(N)nc3c2)C[C@@H](n2ccc3c(N)ncc(F)c32)[C@H](O)[C@@H]1O]

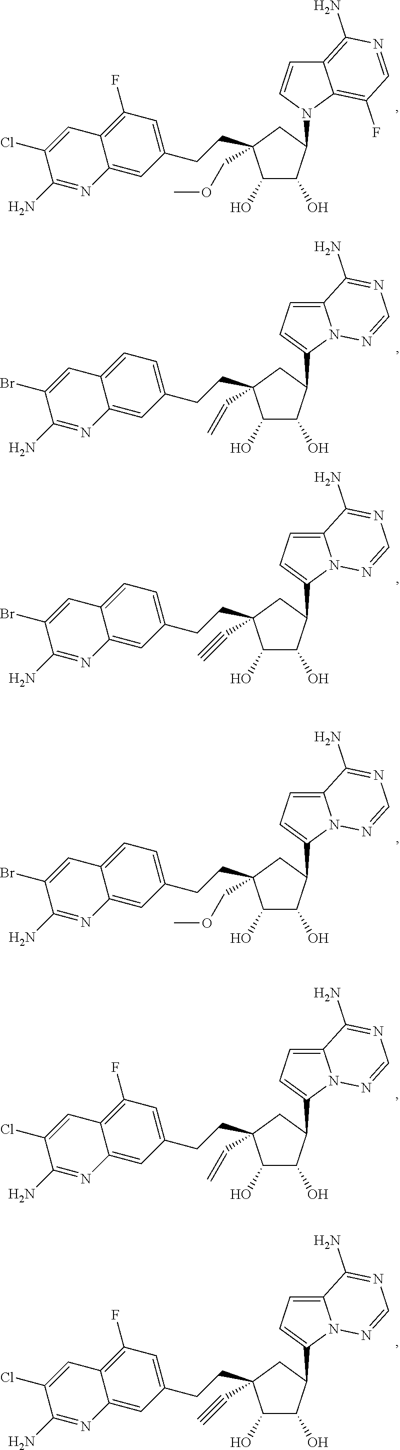

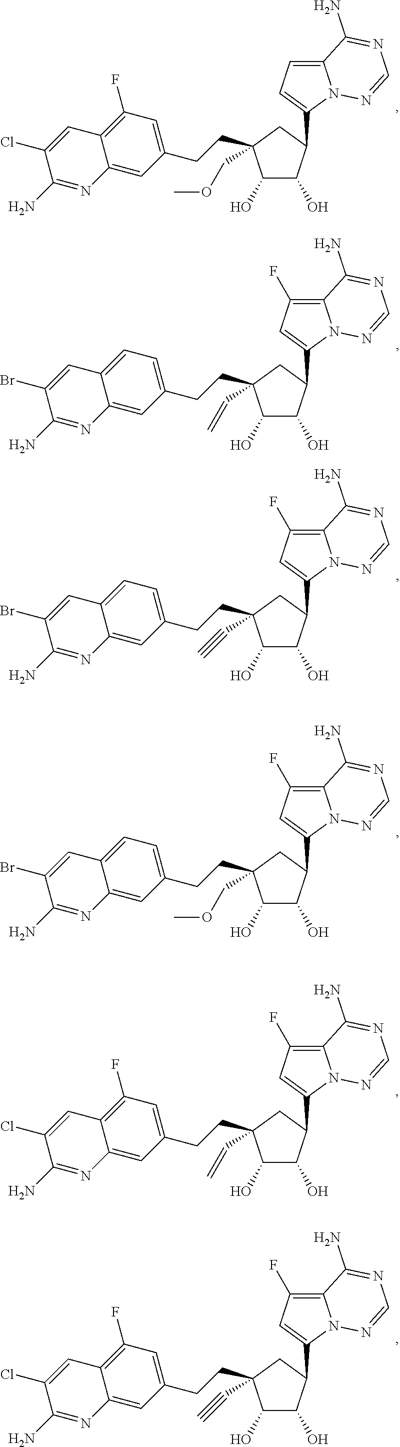

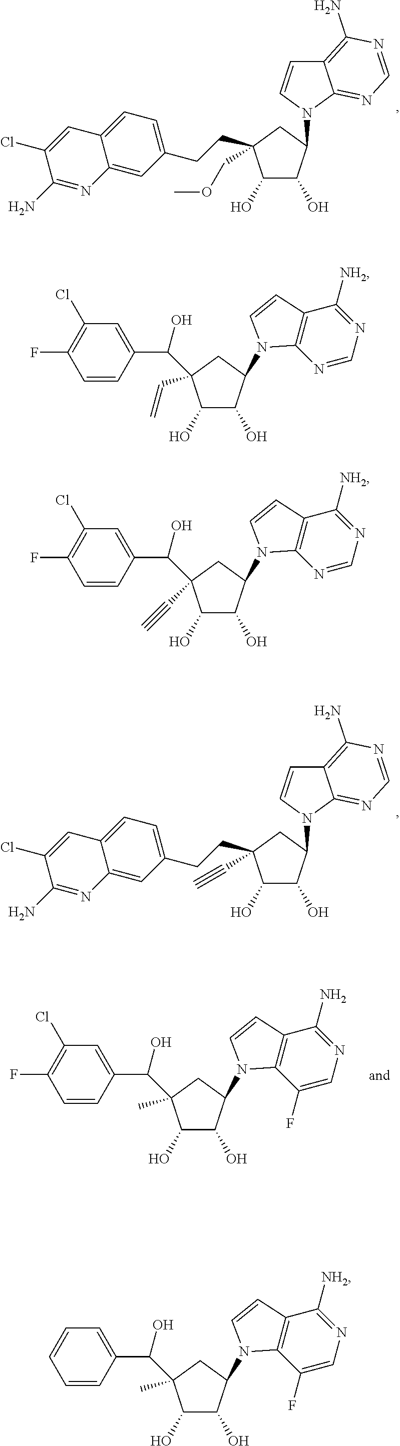 [SMILES: COC[C@]1(CCc2ccc3cc(Cl)c(N)nc3c2)C[C@@H](n2ccc3c(N)ncnc32)[C@H](O)[C@@H]1O ; C=C[C@]1(C(O)c2ccc(F)c(Cl)c2)C[C@@H](n2ccc3c(N)ncnc32)[C@H](O)[C@@H]1O ; C#C[C@]1(C(O)c2ccc(F)c(Cl)c2)C[C@@H](n2ccc3c(N)ncnc32)[C@H](O)[C@@H]1O ; C#C[C@]1(CCc2ccc3cc(Cl)c(N)nc3c2)C[C@@H](n2ccc3c(N)ncnc32)[C@H](O)[C@@H]1O ; C[C@]1(C(O)c2ccc(F)c(Cl)c2)C[C@@H](n2ccc3c(N)ncc(F)c32)[C@H](O)[C@@H]1O ; C[C@]1(C(O)c2ccccc2)C[C@@H](n2ccc3c(N)ncc(F)c32)[C@H](O)[C@@H]1O]

(including pharmaceutically acceptable salts of any of the foregoing).

### Example A

### PRMT5/MEP50 Enzyme Inhibition Assay

### Assay 1

Compounds were tested for inhibition of methyltransferase activity in a radioisotope filter binding assay, similar to previously described in A selective inhibitor of PRMT5 with in vivo and in vitro potency in MCL models. Chan-Penebre et al., Nat Chem Biol. (2015) 11(6):432-7. In the standard PRMT5/MEP50 enzyme inhibition assay, compounds were tested in a 10-dose IC₅₀ mode with 3- or 5-fold serial dilution, in singlet, starting at 1, 10, or 100 μM. Control compound, SAH (S-(5′-Adenosyl)-L-homocysteine), was tested in 10-dose IC₅₀ mode with 3-fold serial dilution starting at 100 μM. Reactions were carried out at 1 μM 3H-SAM (PerkinElmer) and 5 μM histone H₂A as substrates for methyl transfer. Following a 60 min incubation at 30° C., the reaction was stopped with 20% TCA. Each reaction was spotted on a filter plate (MultiScreen FB Filter plate, Millipore) and washed 5 times in PBS, after which scintillation fluid was added and signal was detected in a scintillation counter. Percent enzyme activity was calculated based on no inhibitor DMSO control as 100% activity. EC₅₀ values were determined in GraphPad Prism 8 using the [inhibitor] vs. response—Variable slope (four parameters) function.

### Assay 2

Compounds were tested for inhibition of methyltransferase activity in 384-well plate assay format using mass spectrometry technology. In this enzyme inhibition assay, compounds were tested in a 11-dose IC₅₀ mode with 3-serial dilution, in duplicate, starting at 1, 10, or 100 μM. Reactions were carried out at 1 μM SAM and 0.1 μM histone H4 1-21 peptide as substrates for methyl transfer. Following an 18-hour incubation at rt, the reaction was stopped with 0.5% formic acid. Products of the reaction were captured while unreacted substrates were washed away, prior to MALDI mass spectrometry detection and analysis. Percent enzyme activity was calculated based on no inhibitor DMSO control as 100% activity. EC₅₀ values were determined in GraphPad Prism 8 using the [inhibitor] vs. response—Variable slope (four parameters) function.

The IC₅₀ values were derived from the procedure as described herein and are shown in Table 1. Compounds of Formula (I) show activity in this assay. A value of ‘A’ in the table below indicates an IC₅₀ of <1 nM, a value of ‘B’ indicates an IC₅₀≥1 nM to ≤100 nM, and a value of ‘C’ indicates an IC₅₀ value of >100 nM.

TABLE 1

| | Assay 1 | Assay 2 |
|---|---|---|
| Compound: | IC₅₀ | IC₅₀ |
| 1 | A | A |
| 2 | A | A |
| 3 | C | C |
| 4 | A | A |
| 5 | A | A |
| 6 | B | B |
| 7 | A | B |
| 8 | B | C |
| 9 | A | A |
| 10 | A | A |
| 11 | C | B |
| 12 | A | A |
| 13 | A | A |
| 14 | A | A |
| 15 | — | A |
| 16 | — | A |
| 17 | — | C |
| 18 | — | A |
| 19 | — | A |
| 20 | — | B |
| 20-A | — | C |
| 21 | — | A |
| 21-A | — | B |
| 22 | — | A |
| 23 | — | A |
| 24 | — | A |
| 25-A | — | C |
| 26 | — | C |
| 26-A | — | C |
| 27 | — | A |
| 28 | — | A |
| 29 | — | A |
| 30 | — | A |
| 31 | — | A |
| 34 | — | A |
| 35 | — | A |
| 36 | — | A |
| 37 | — | A |
| 38 | — | A |
| 39 | — | A |
| 40 | — | A |
| 41 | — | A |
| 42 | — | A |
| 43 | — | A |
| 44 | — | A |
| 45 | — | A |
| 46 | — | A |
| 47 | — | A |
| 48 | — | A |
| 49 | — | A |
| 50 | — | A |
| 51 | — | A |
| 52 | — | A |
| 53 | — | A |
| 54 | — | A |
| 55 | | C |
| 56 | | A |
| 57 | | A |
| 58 | | A |
| 59 | | A |
| 60 | | A |

### Example B

### Cell Proliferation Assays

HepG2 hepatoma cells (ATCC, HB-8065) were maintained in DMEM with high glucose (Lonza, 12-914F) supplemented with 10% fetal bovine serum (FBS; Biowest, S181B-500) and 2 mM L-glutamine (Biowest, X0551-100) at 37° C. and 5% CO₂. A549 lung carcinoma cells (ATCC, CCL-185) were maintained in F-12K medium (ThermoFisher 21127030) supplemented with 10% FBS at 37° C. and 5% CO₂. Exponentially growing HepG2 or A549 were plated in white, clear-bottom 96-well plates (Corning, 3903) at a cell density of 2000 (HepG2) or 350 (A549) cells per well in 199 μL of HepG2 medium. Next, 1 μL of a 5-fold 9-point dilution series of test compound in DMSO was added to the different wells. Cells were incubated at 37° C. 5% CO₂ for 7 days. Cell viability was assessed on day 7 with the CellTiter-Glo 2.0 Cell Viability assay kit (Promega, G9243) to quantify the intracellular amounts of ATP: first, 100 μL of cell culture medium was removed from each well, next 100 μL of CellTiter-Glo® 2.0 Reagent was added and plates were shaken for 2 minutes on an orbital shaker. After 10 minutes stabilization at room temperature, read-out was performed on a ThermoFisher VarioSkan Lux plate reader. Counts were normalized to DMSO control (0% inhibition) and the cytotoxic control (100% inhibition) and EC₅₀ values were determined in GraphPad Prism 8 using the [Agonist] vs. response—Variable slope (four parameters) function.

The EC₅₀ values were derived from the procedure as described herein and are shown in Table 2. As shown by the data of Table 2, compounds of Formula (I) have activity in this assay. A value of ‘A’ in the table below indicates an EC₅₀ of <20 nM, a value of ‘B’ indicates an EC₅₀≥20 nM to <100 nM, a value of ‘C’ indicates an EC₅₀ value of EC₅₀≥100 nM to <1000 nM and a value of ‘D’ indicates an EC₅₀≥1000 nM

TABLE 2
HepG2 and A549

| | EC₅₀ | EC₅₀ |
|---|---|---|
| Compound | HepG2 | A549 |
| 1 | A | A |
| 2 | A | A |
| 3 | C | C |
| 4 | A | A |
| 5 | C | C |
| 6 | D | D |
| 7 | C | C |
| 8 | C | D |
| 9 | B | B |
| 10 | A | A |
| 11 | C | D |
| 12 | A | A |
| 13 | A | A |
| 14 | A | A |
| 15 | C | C |
| 16 | D | D |
| 17 | D | D |
| 18 | A | A |
| 19 | A | A |
| 20 | A | B |
| 20-A | C | C |
| 21 | A | A |
| 21-A | D | D |
| 22 | C | C |
| 23 | A | A |
| 24 | B | B |
| 25-A | D | D |
| 26 | B | C |
| 26-A | D | D |
| 27 | B | B |
| 28 | B | C |
| 29 | A | B |
| 30 | A | A |
| 31 | C | D |
| 32 | A | A |
| 33 | A | A |
| 34 | A | A |
| 35 | A | A |
| 36 | A | A |
| 37 | A | A |
| 38 | A | A |
| 39 | B | B |
| 40 | A | A |
| 41 | A | A |
| 42 | A | A |
| 43 | B | A |
| 44 | C | B |
| 45 | C | C |
| 46 | C | C |
| 47 | C | B |
| 48 | A | A |
| 49 | A | A |
| 50 | A | A |
| 51 | A | A |
| 52 | A | A |
| 53 | A | A |
| 54 | A | A |
| 55 | B | B |
| 56 | A | A |
| 57 | A | A |
| 58 | A | A |
| 59 | A | A |
| 61 | A | A |
| 62 | | A |
| 63 | | A |
| 64 | | A |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure provided herein.

1. A compound of Formula (I), or a pharmaceutically acceptable salt thereof, having the structure: 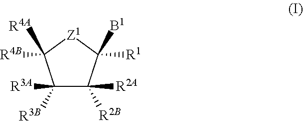
wherein:
B¹ is an optionally substituted 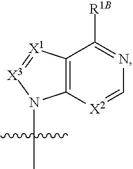
an optionally substituted 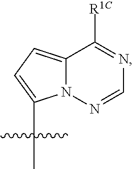
an optionally substituted 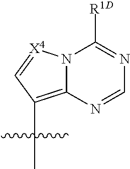
or an optionally substituted 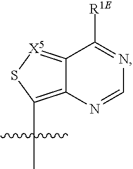
wherein X¹ is N or CR^{C1}; X² is N or CR^{C2}; X³ is N or CR^{C3}; X⁴ is N or CR^{C4}; X⁵ is N or CR^{C5}; and R^{C1}, R^{C2}, R^{C3}, R^{C4} and R^{C5} are independently hydrogen, halogen or an unsubstituted C₁₋₄ alkyl;
R^{1B}, R^{1C}, R^{1D} and R^{1E} are independently hydrogen, halogen, hydroxy, an unsubstituted C₁₋₄ alkyl, an unsubstituted C₂₋₄ alkenyl, an unsubstituted C₃-C₆ cycloalkyl, an unsubstituted C₁₋₄ alkoxy or NR^{A1}R^{A2}; and
R^{A1} and R^{A2} are independently selected from the group consisting of hydrogen, hydroxy, an unsubstituted C₁₋₄ alkyl, an unsubstituted C₁₋₄ alkoxy and —C(═O)R^{C6}, wherein R^{C6} is hydrogen, an unsubstituted C₁₋₄ alkyl or an unsubstituted C₃₋₄ monocyclic cycloalkyl;
R¹ is hydrogen or an unsubstituted C₁₋₄ alkyl;
R^{2A} is hydrogen or an unsubstituted C₁₋₄ alkyl;
R^{2B} is halogen, OH, —O—C(═O)—C₁₋₄ alkyl or —O—C(═O)—CH(R¹′)—NH₂, wherein R¹′ is H, —CH₃, —CH(CH₃)₂, —CH₂—CH(CH₃)₂ or —CH(CH₃)—CH(CH₃)₂;
R^{3A} is hydrogen, an unsubstituted or a substituted C₁₋₄ alkyl, an unsubstituted or a substituted C₂₋₄ alkenyl or an unsubstituted or a substituted C₂₋₄ alkynyl, wherein when the C₁₋₄ alkyl, the C₂₋₄ alkenyl and the C₂₋₄ alkynyl are substituted, each is independently substituted with 1 or more fluoros;
R^{3B} is halogen, OH, —O—C(═O)—C₁₋₄ alkyl or —O—C(═O)—CH(R¹″)—NH₂, wherein R¹″ is H, —CH₃, —CH(CH₃)₂, —CH₂—CH(CH₃)₂ or —CH(CH₃)—CH(CH₃)₂;
R^{4A} is —(CR^{D1}R^{E1})(CR^{D2}R^{E2})n-R^{F1}, —(CR^{G1}R^{H1})—O—R^{J1}, —O—(CR^{K1}R^{L1})—R^{M1} or —(CR^{N1}R^{O1})p-R^{P1};
wherein R^{D1}, R^{E1}, R^{D2} and R^{E2} are independently selected from the group consisting of hydrogen, halogen, hydroxy and an unsubstituted C₁₋₃ alkyl; n is 0 or 1; and R^{F1} is an unsubstituted or a substituted aryl, an unsubstituted or a substituted heteroaryl or an unsubstituted or a substituted heterocyclyl; or
R^{D1} and R^{E1} are taken together with the carbon to which R^{D1} and R^{E1} are attached to form an unsubstituted cyclopropyl ring; and R^{D2} and R^{E2} are independently selected from the group consisting of hydrogen, halogen, hydroxy and an unsubstituted C₁₋₃ alkyl; n is 1; and R^{F1} is an unsubstituted or a substituted aryl, an unsubstituted or a substituted heteroaryl or an unsubstituted or a substituted heterocyclyl; or
R^{D1} and R^{E2} are independently selected from the group consisting of hydrogen, halogen, hydroxy and an unsubstituted C₁₋₃ alkyl; R^{E1} and R^{D2} are taken together with the carbon to which R^{E1} and R^{D2} are attached to form an unsubstituted cyclopropyl ring; n is 1; and R^{F1} is an unsubstituted or a substituted aryl, an unsubstituted or a substituted heteroaryl or an unsubstituted or a substituted heterocyclyl; or
R^{D1} and R^{E2} are independently selected from the group consisting of hydrogen, halogen and hydroxy; R^{E1} and R^{D2} together form a double bond; n is 1; and R^{F1} is an unsubstituted or a substituted aryl, an unsubstituted or a substituted heteroaryl or an unsubstituted or a substituted heterocyclyl;
R^{G1}, R^{H1}, R^{K1}, R^{L1}, R^{N1} and R^{O1} are independently selected from the group consisting of hydrogen, halogen, hydroxy and an unsubstituted C₁₋₃ alkyl; R^{J1} and R^{M1} are independently an unsubstituted or a substituted aryl, an unsubstituted or a substituted heteroaryl or an unsubstituted or a substituted heterocyclyl; R^{P1} is an unsubstituted or a substituted heteroaryl; and p is 3 or 4;
R^{4B} is hydrogen, halogen, cyano, azido, —C(═O)NH₂, an unsubstituted or a substituted C₁₋₄ alkyl, an unsubstituted or a substituted C₂₋₄ alkenyl, an unsubstituted or a substituted C₂₋₄ alkynyl or an unsubstituted or a substituted C₃-C₄ cycloalkyl,
wherein when the C₁₋₄ alkyl is substituted, the C₁₋₄ alkyl is substituted with 1 or more substituents independently selected from the group consisting of halogen, OH, OCH₃ and cyano, and
wherein when the C₂₋₄ alkenyl is substituted, the C₂₋₄ alkenyl is substituted independently with 1 or more halogens;
Z¹ is CR^{5A}R^{5B}, O, S or N (an unsubstituted C₁₋₄ alkyl);
R^{5A} and R^{5B} are independently hydrogen, halogen, cyano or an unsubstituted or a substituted C₁₋₄ alkyl, wherein when the C₁₋₄ alkyl is substituted, the C₁₋₄ alkyl is substituted with 1 or more substituents independently selected from the group consisting of fluoro and hydroxy; or
R^{5A} and R^{5B} together with the carbon R^{5A} and R^{5B} are attached form a double bond optionally substituted with one or two halogen, R^{5A} and R^{5B} together with the carbon R^{5A} and R^{5B} are attached form an unsubstituted cyclopropyl or R^{5A} and R^{5B} together with the carbon R^{5A} and R^{5B} are attached form an unsubstituted or a substituted oxetane, wherein when the oxetane is substituted, the oxetane is substituted independently with 1 or 2 halogens; or
R^{2A} and R^{2B} together with the carbon R^{2A} and R^{2B} are attached form a 3, 4 or 5 membered monocyclic cycloalkyl or a 3, 4 or 5 membered monocyclic heterocyclyl; or
R^{3A} and R^{3B} together with the carbon R^{3A} and R^{3B} are attached form a 3, 4 or 5 membered monocyclic cycloalkyl or a 3, 4 or 5 membered monocyclic heterocyclyl; or
R^{4B} and R^{3B} together with the carbon R^{4B} and R^{3B} are attached form an unsubstituted oxetane; or
R^{4B} and R^{5B} together with the carbon R^{4B} and R^{5B} are attached form an unsubstituted cyclopropyl; or
R¹ and R^{5B} together with the carbon R¹ and R^{5B} are attached form an unsubstituted cyclopropyl; or
when Z¹ is O, then R^{2B} and R^{4B} are connected via —(CH₂)y-O—, wherein y is 1 or 2, 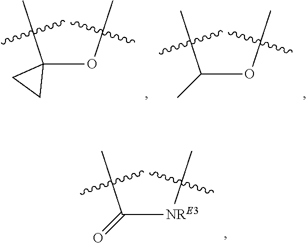
wherein R^{E3} is hydrogen or an unsubstituted C₁₋₇ alkyl or

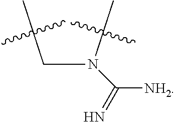 [SMILES: *CN(*)C(=N)N]

2. The compound of claim 1, wherein Z¹ is CR^{5A}R^{5B}.

3.-16. (canceled)

17. The compound of claim 1, wherein R^{2A} is hydrogen.

18. (canceled)

19. The compound of claim 1, wherein R^{2B} is OH.

20. (canceled)

21. (canceled)

22. (canceled)

23. (canceled)

24. (canceled)

25. The compound of claim 1, wherein R^{3A} is hydrogen.

26. (canceled)

27. (canceled)

28. (canceled)

29. (canceled)

30. (canceled)

31. (canceled)

32. The compound of claim 1, wherein R^{3B} is OH.

33. (canceled)

34. (canceled)

35. (canceled)

36. (canceled)

37. (canceled)

38. The compound of claim 1, wherein R^{4A} is —(CR^{D1}R^{E1})(CR^{D2}R^{E2})n-R^{F1}.

39.-104. (canceled)

105. The compound of claim 1, wherein R^{4B} is an unsubstituted C₂₋₄ alkenyl or an unsubstituted C₂₋₄ alkynyl.

106. (canceled)

107. The compound of claim 1, wherein R^{4B} is an unsubstituted C₁₋₄ alkyl or a substituted C₁₋₄ alkyl substituted with 1 or more substituents independently selected from the group consisting of halogen, OH, OCH₃ and cyano, a substituted C₂₋₄ alkenyl substituted independently with 1 or more halogens or a substituted C₂₋₄ alkynyl.

108. (canceled)

109. (canceled)

110. (canceled)

111. The compound of claim 1, wherein R^{4B} is an unsubstituted C₂₋₄ alkenyl or an unsubstituted C₂₋₄ alkynyl.

112. (canceled)

113. (canceled)

114. (canceled)

115. The compound of claim 1, wherein B¹ is an optionally substituted 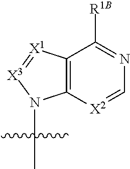
or an optionally substituted

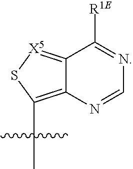

116. (canceled)

117. (canceled)

118. (canceled)

119. (canceled)

120. (canceled)

121. (canceled)

122. The compound of claim 1, wherein B¹ is an optionally substituted 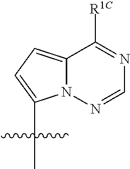
or an optionally substituted

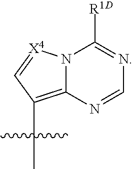

123.-141. (canceled)

142. The compound of claim 1, wherein B¹ is selected from the group consisting of:

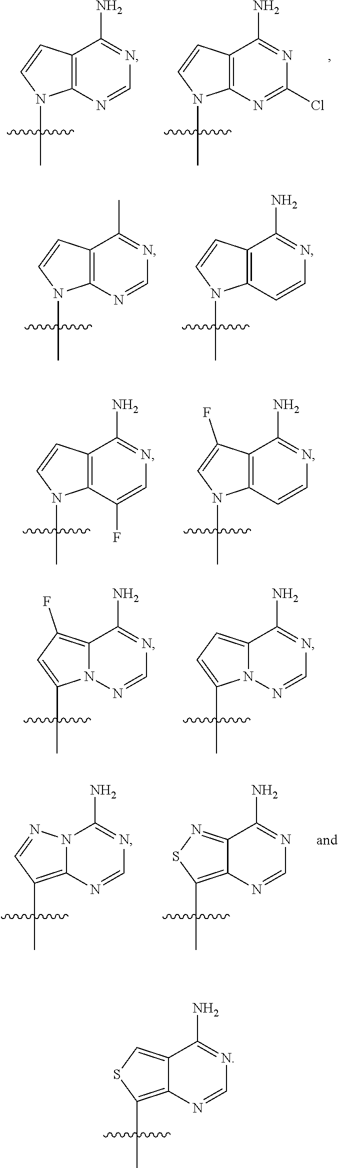

143. (canceled)

144. (canceled)

145. (canceled)

146. (canceled)

147. (canceled)

148. (canceled)

149. The compound of claim 1, wherein the compound is 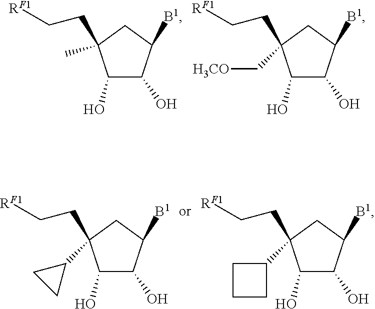
or a pharmaceutically acceptable salt thereof.

150. (canceled)

151. The compound of claim 1, wherein the compound is 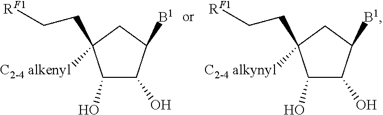
or a pharmaceutically acceptable salt thereof.

152. (canceled)

153. (canceled)

154. (canceled)

155. (canceled)

156. The compound of claim 149, wherein B¹ is

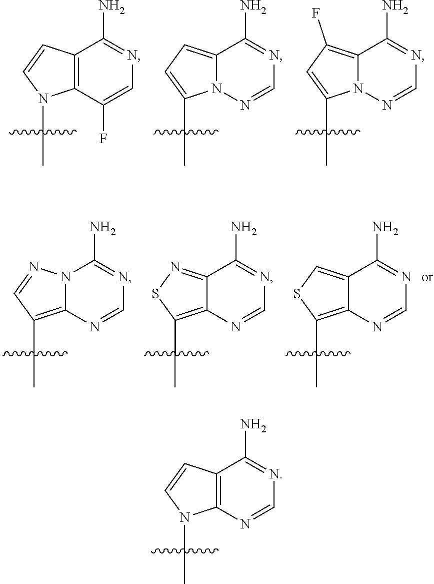

157. (canceled)

158. (canceled)

159. (canceled)

160. (canceled)

161. (canceled)

162. (canceled)

163. The compound of claim 149, wherein R^{F1} is selected from the group consisting of:

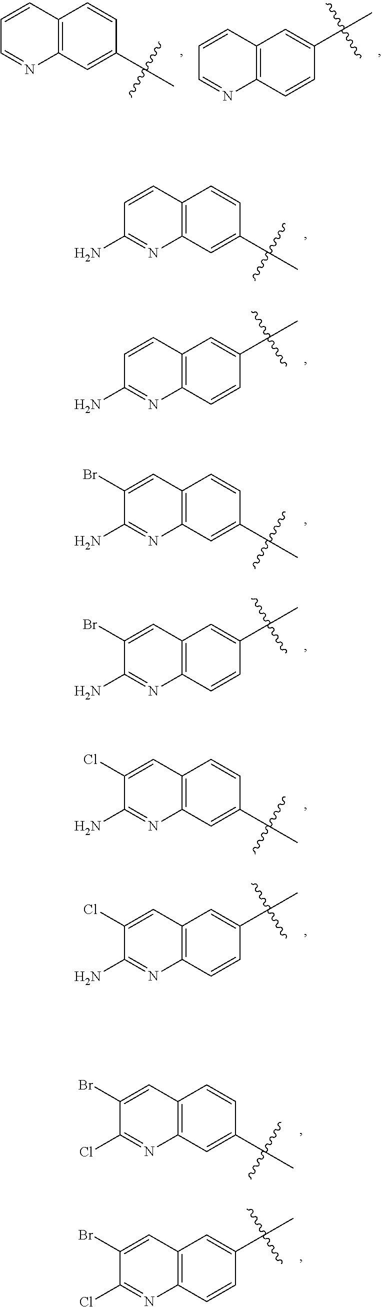

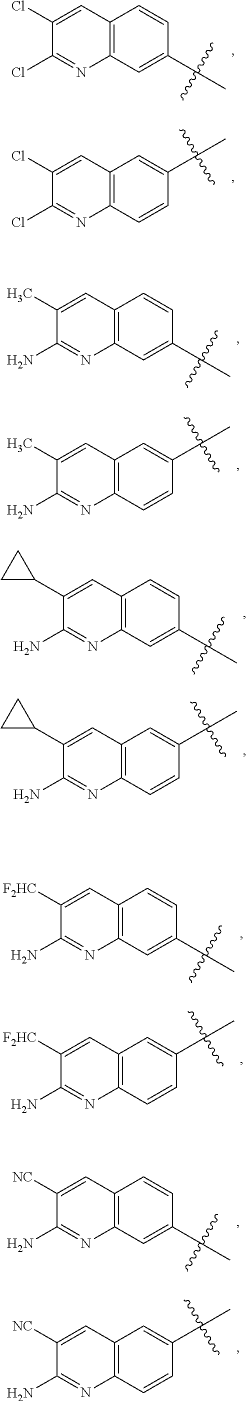

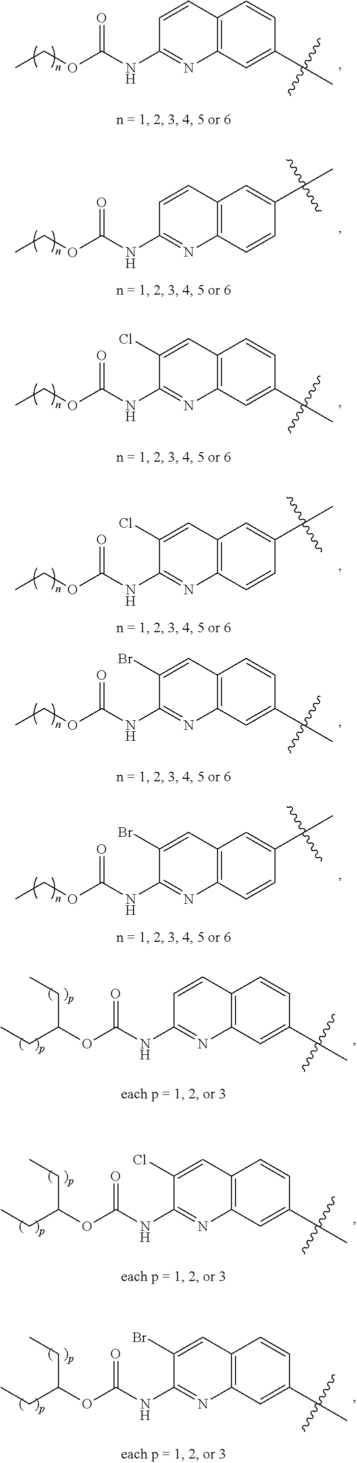

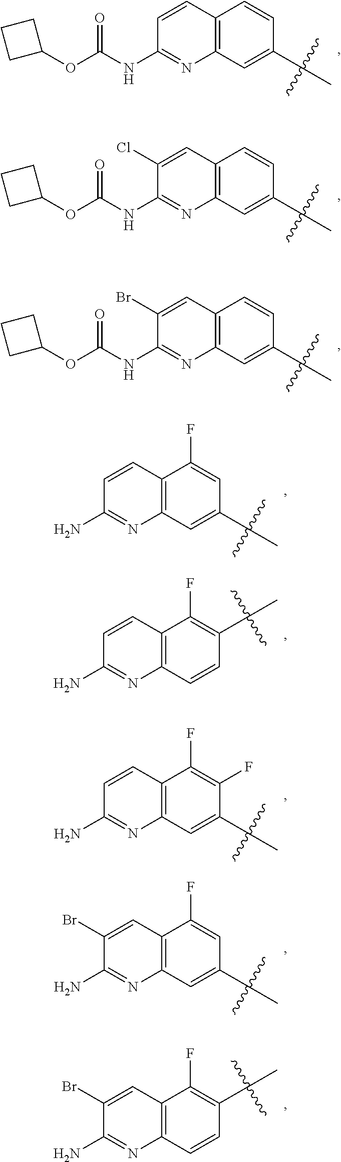

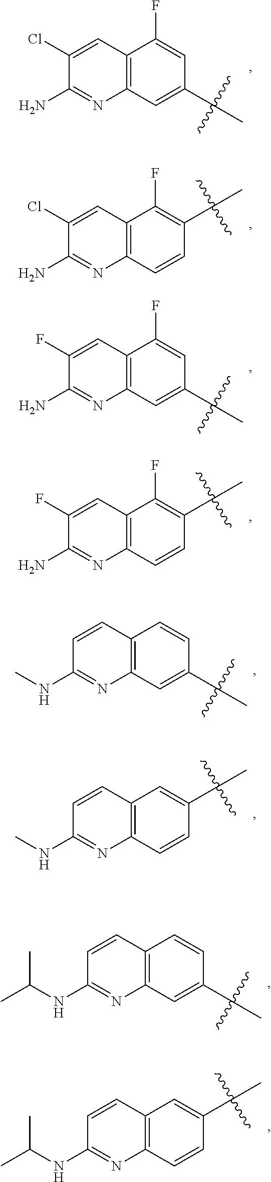

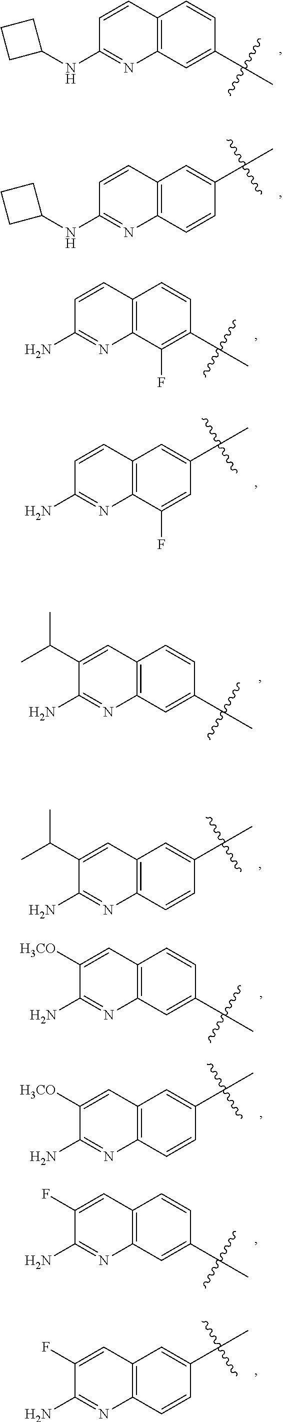

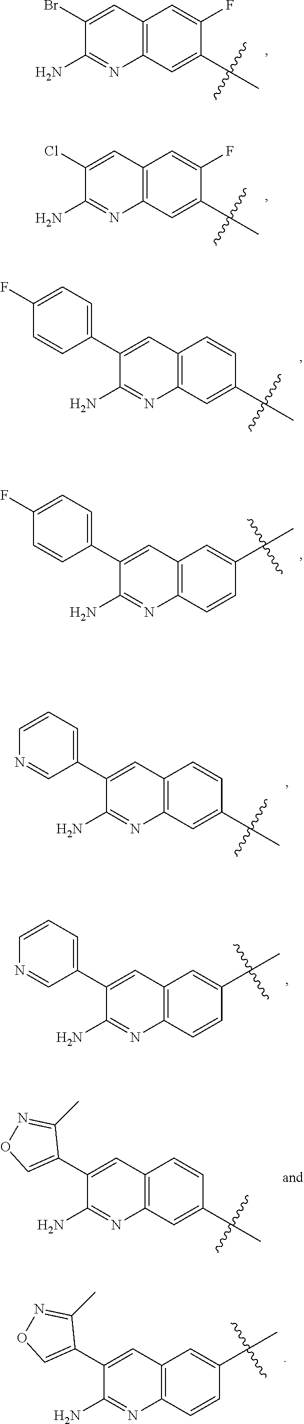

164. (canceled)

165. The compound of claim 1, wherein the compound is selected from the group consisting of: 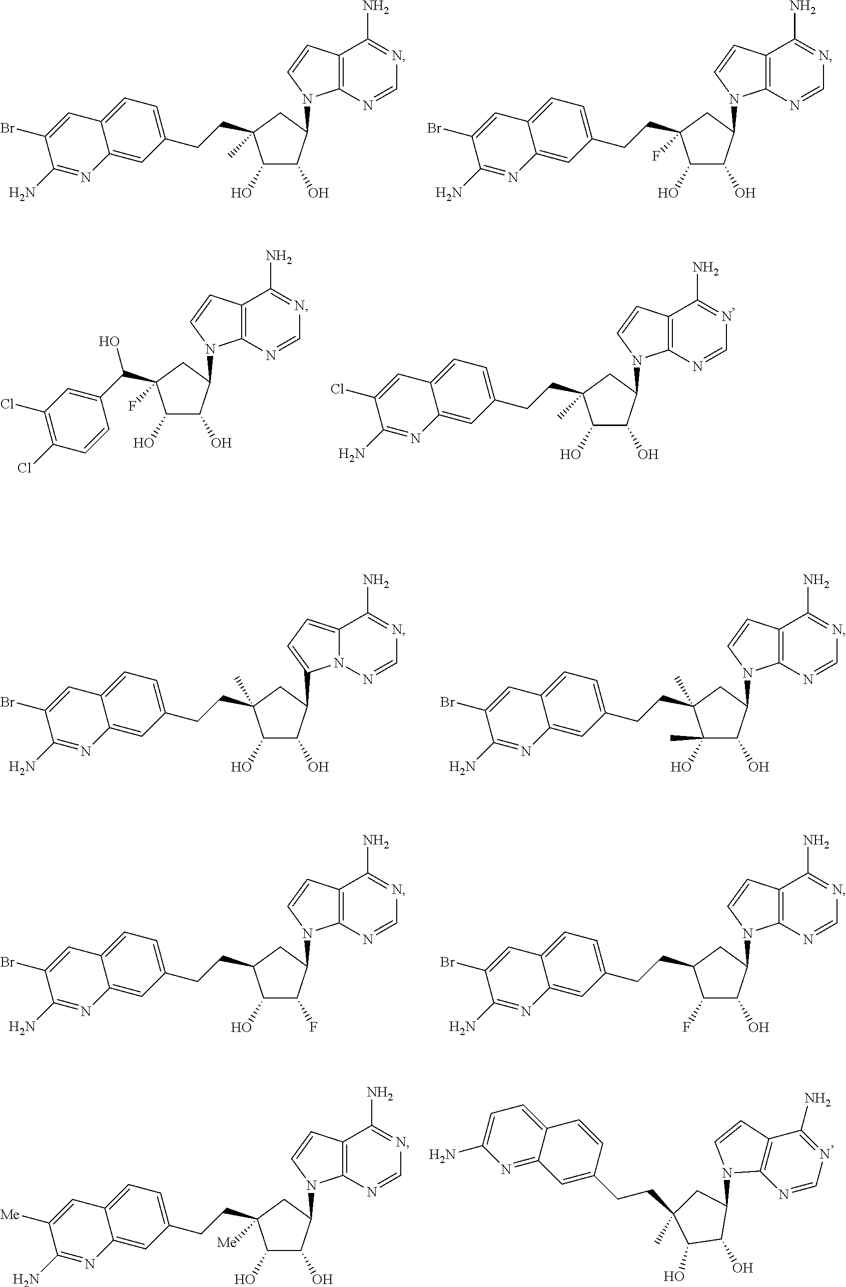 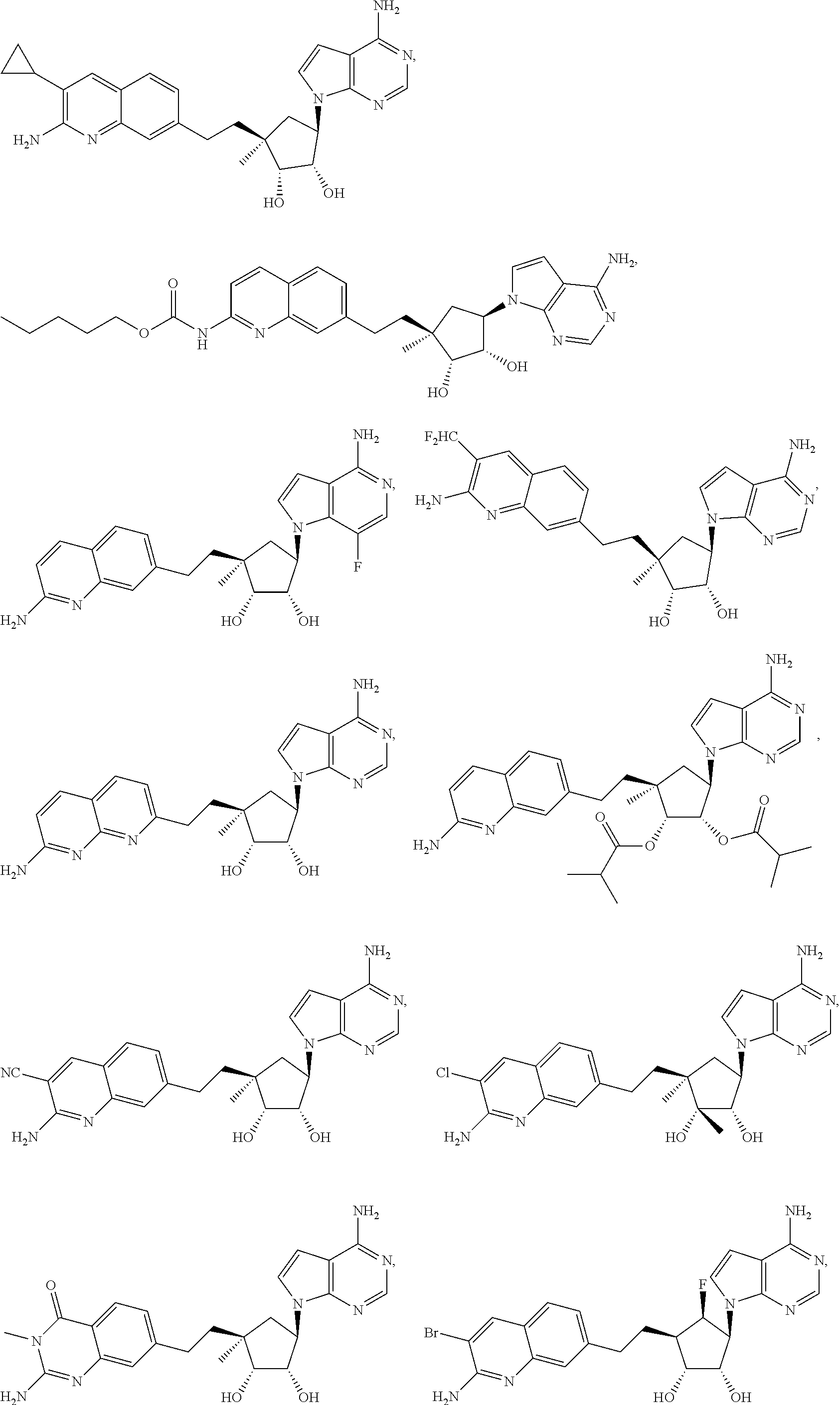 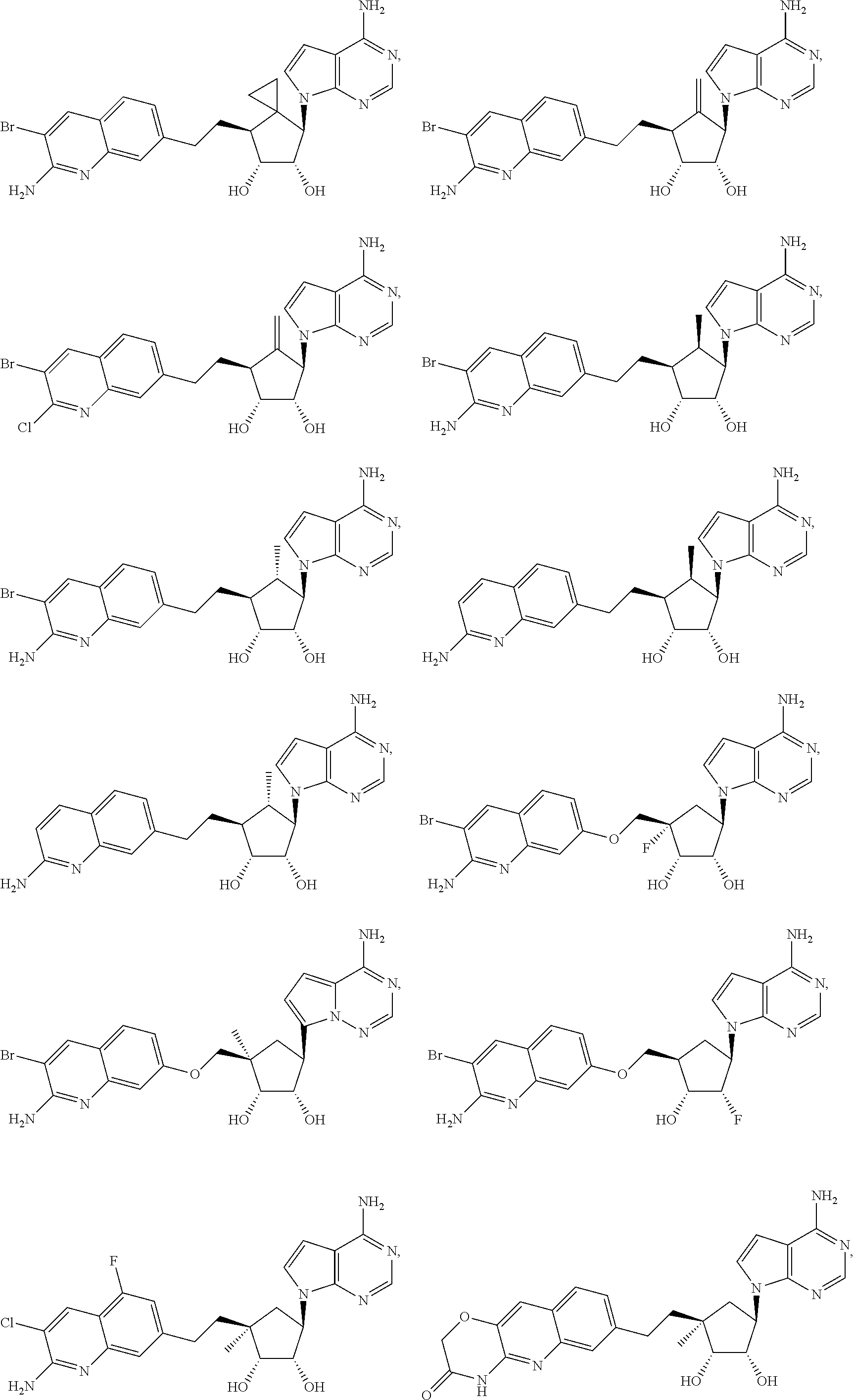 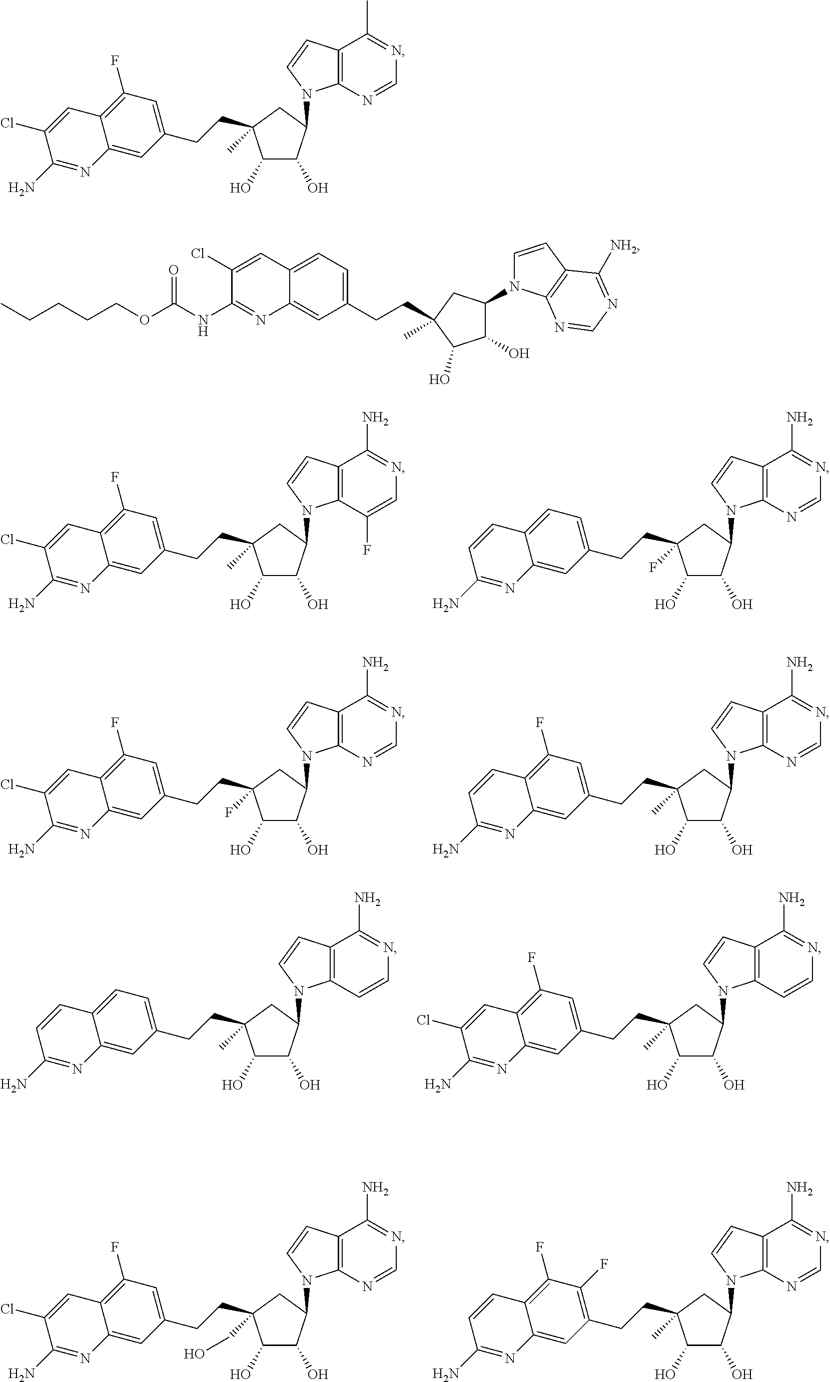 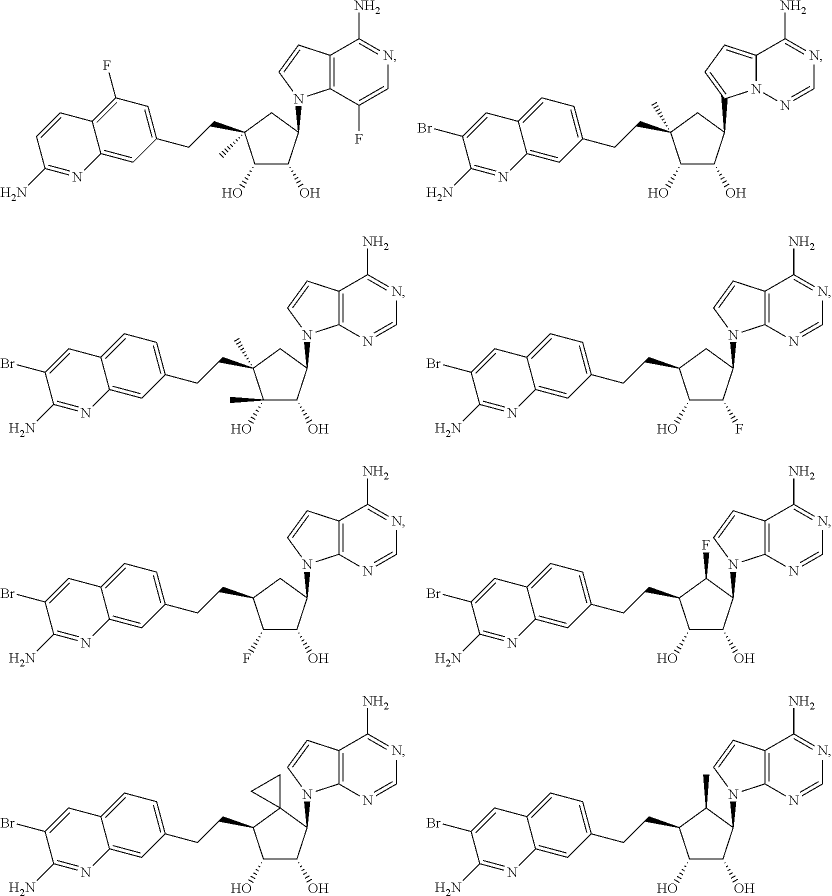 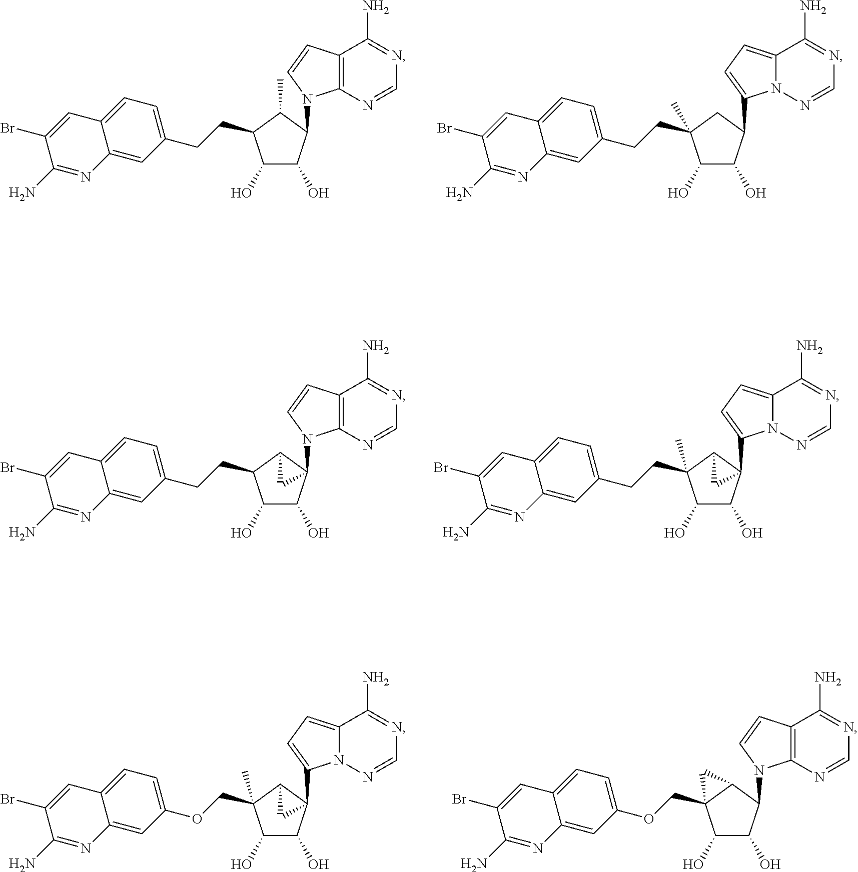 [SMILES: C[C@H]1[C@H](CCc2ccc3cc(Br)c(N)nc3c2)[C@@H](O)[C@@H](O)[C@@H]1n1ccc2c(N)ncnc21 ; C[C@]1(CCc2ccc3cc(Br)c(N)nc3c2)C[C@@H](c2ccc3c(N)ncnn23)[C@H](O)[C@@H]1O ; Nc1nc2cc(CC[C@@H]3[C@@H](O)[C@@H](O)[C@]4(n5ccc6c(N)ncnc65)C[C@@H]34)ccc2cc1Br ; C[C@@]1(CCc2ccc3cc(Br)c(N)nc3c2)[C@@H](O)[C@@H](O)[C@]2(c3ccc4c(N)ncnn34)C[C@@H]12 ; C[C@@]1(COc2ccc3cc(Br)c(N)nc3c2)[C@@H](O)[C@@H](O)[C@]2(c3ccc4c(N)ncnn34)C[C@@H]12 ; Nc1nc2cc(OC[C@@]34C[C@@H]3[C@@H](n3ccc5c(N)ncnc53)[C@H](O)[C@@H]4O)ccc2cc1Br] 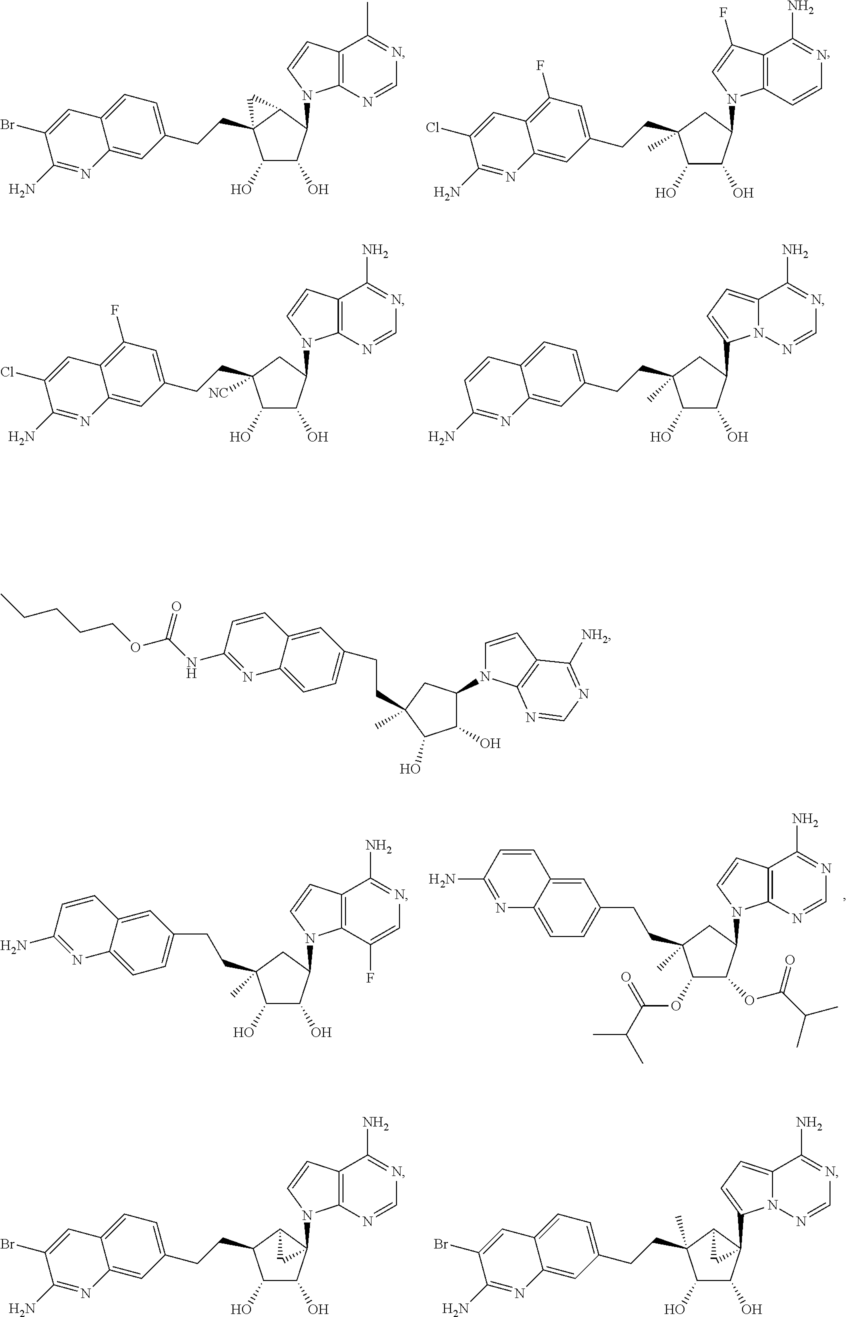 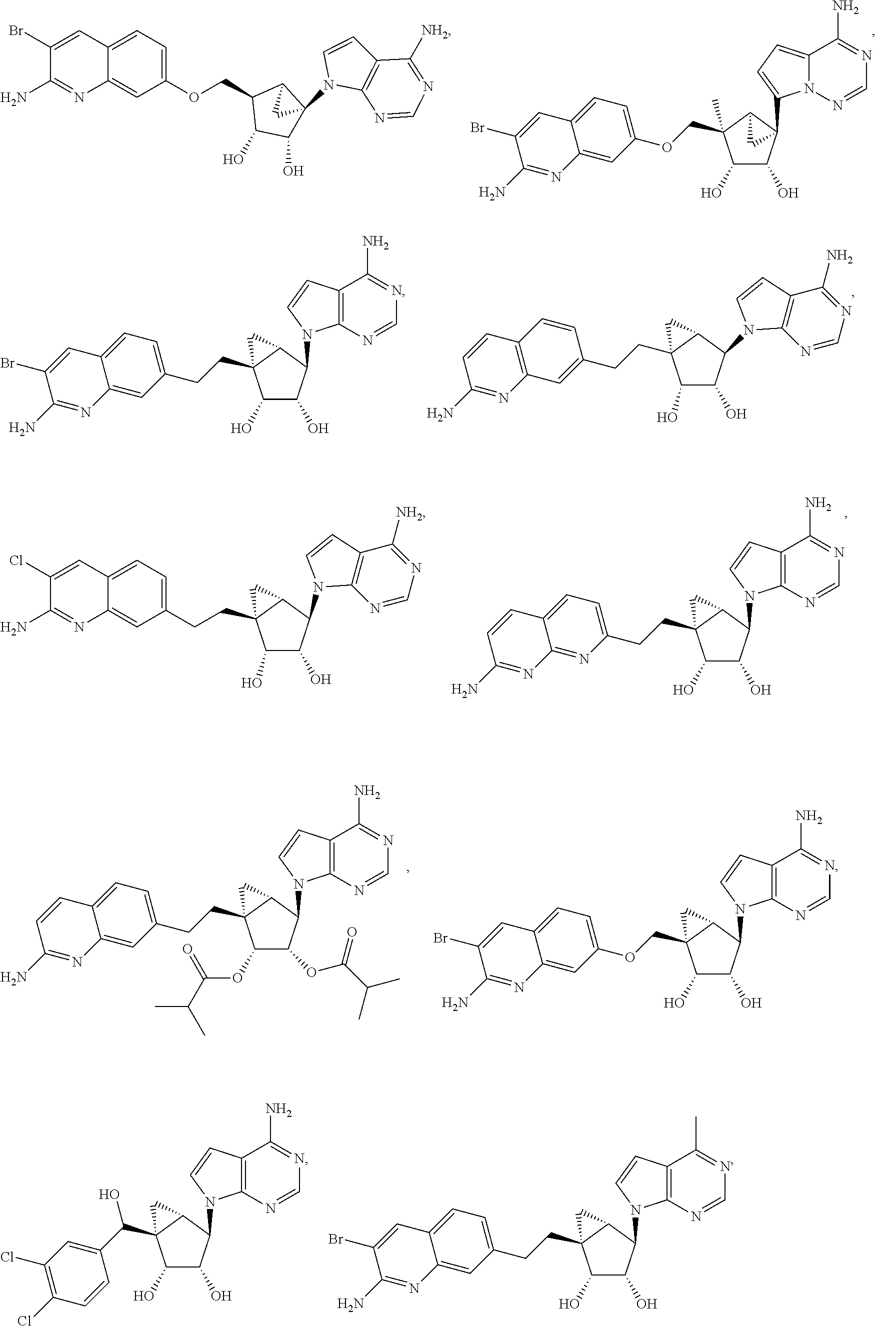 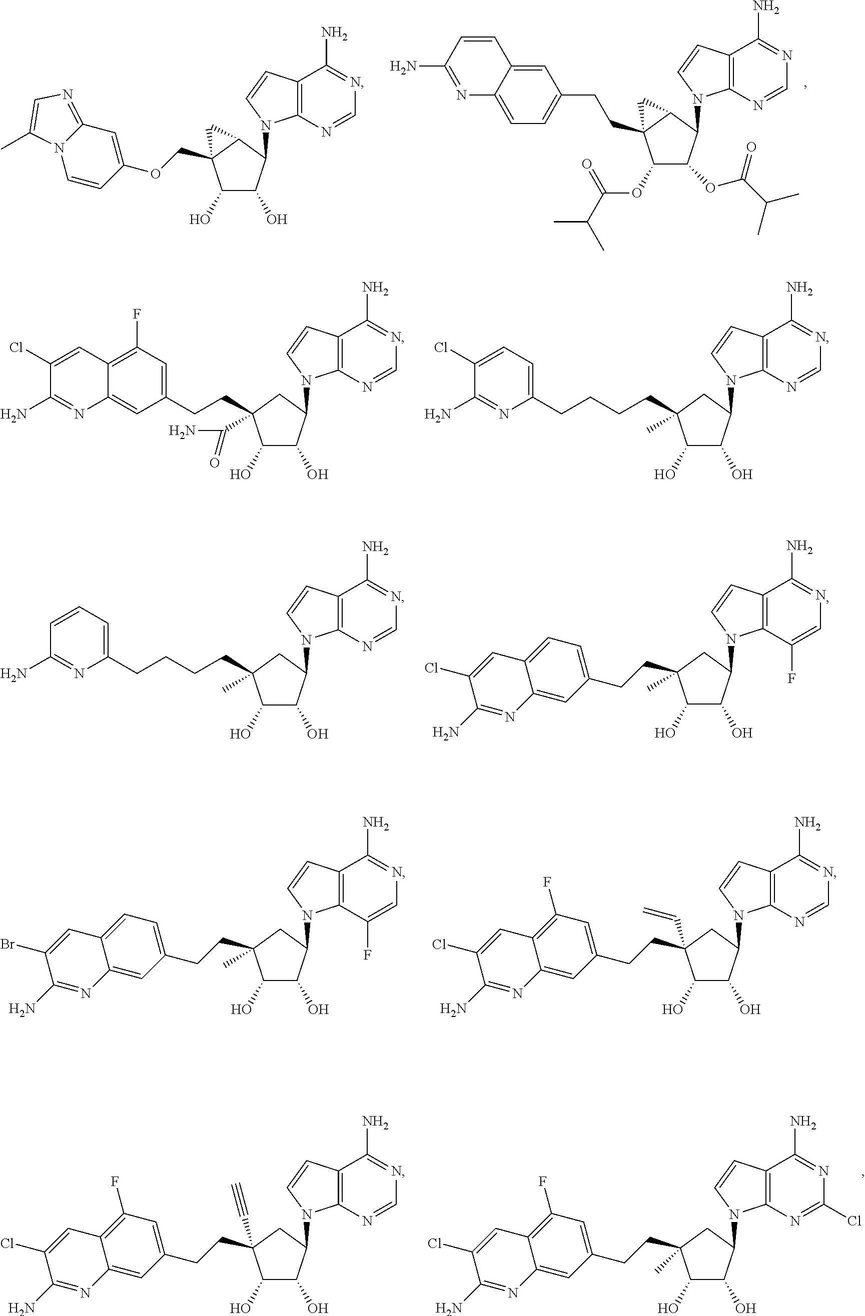 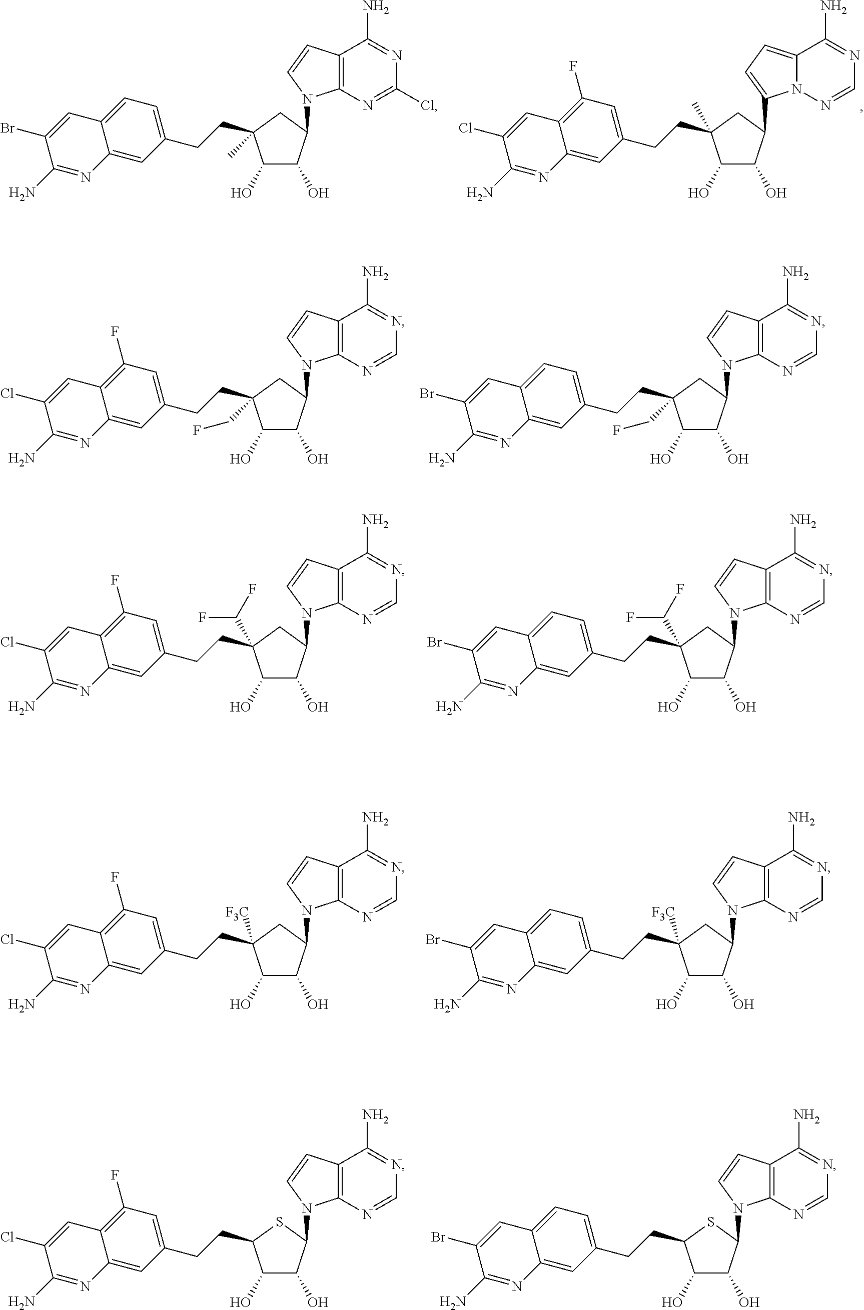 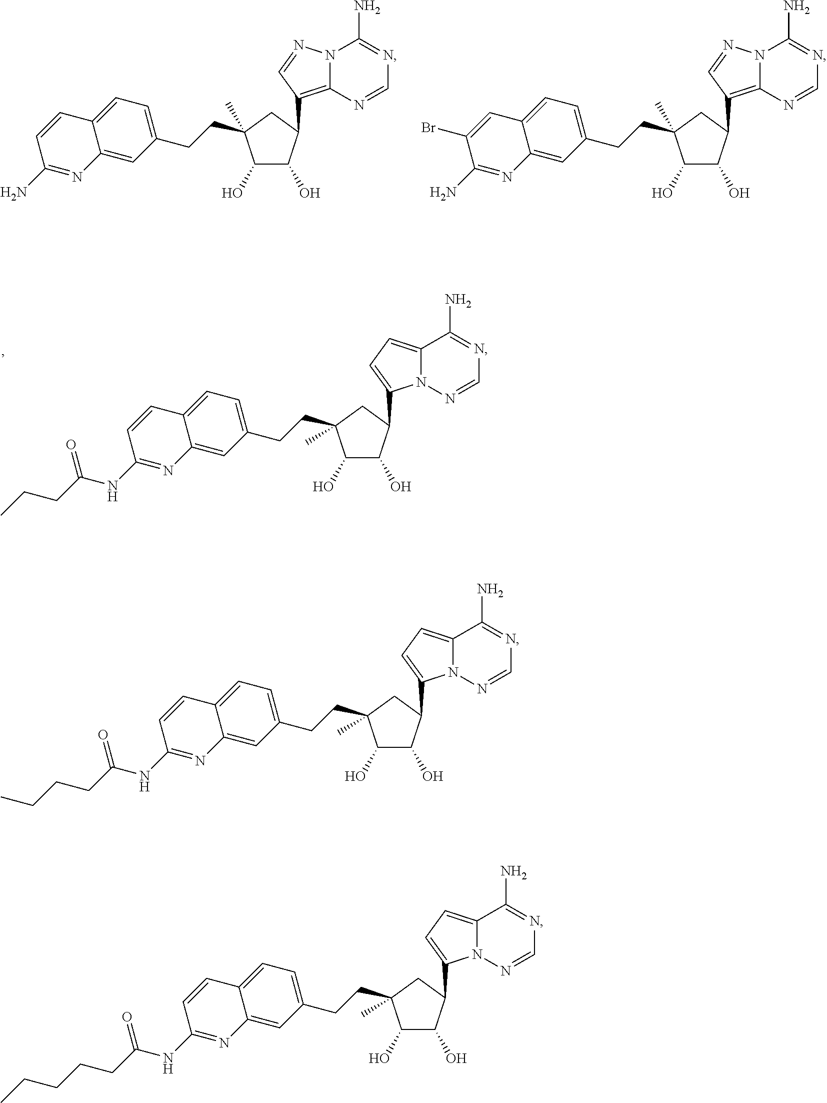 [SMILES: C[C@]1(CCc2ccc3ccc(N)nc3c2)C[C@@H](c2cnn3c(N)ncnc23)[C@H](O)[C@@H]1O ; C[C@]1(CCc2ccc3cc(Br)c(N)nc3c2)C[C@@H](c2cnn3c(N)ncnc23)[C@H](O)[C@@H]1O ; CCCC(=O)Nc1ccc2ccc(CC[C@@]3(C)C[C@@H](c4ccc5c(N)ncnn45)[C@H](O)[C@@H]3O)cc2n1 ; CCCCC(=O)Nc1ccc2ccc(CC[C@@]3(C)C[C@@H](c4ccc5c(N)ncnn45)[C@H](O)[C@@H]3O)cc2n1 ; CCCCCC(=O)Nc1ccc2ccc(CC[C@@]3(C)C[C@@H](c4ccc5c(N)ncnn45)[C@H](O)[C@@H]3O)cc2n1] 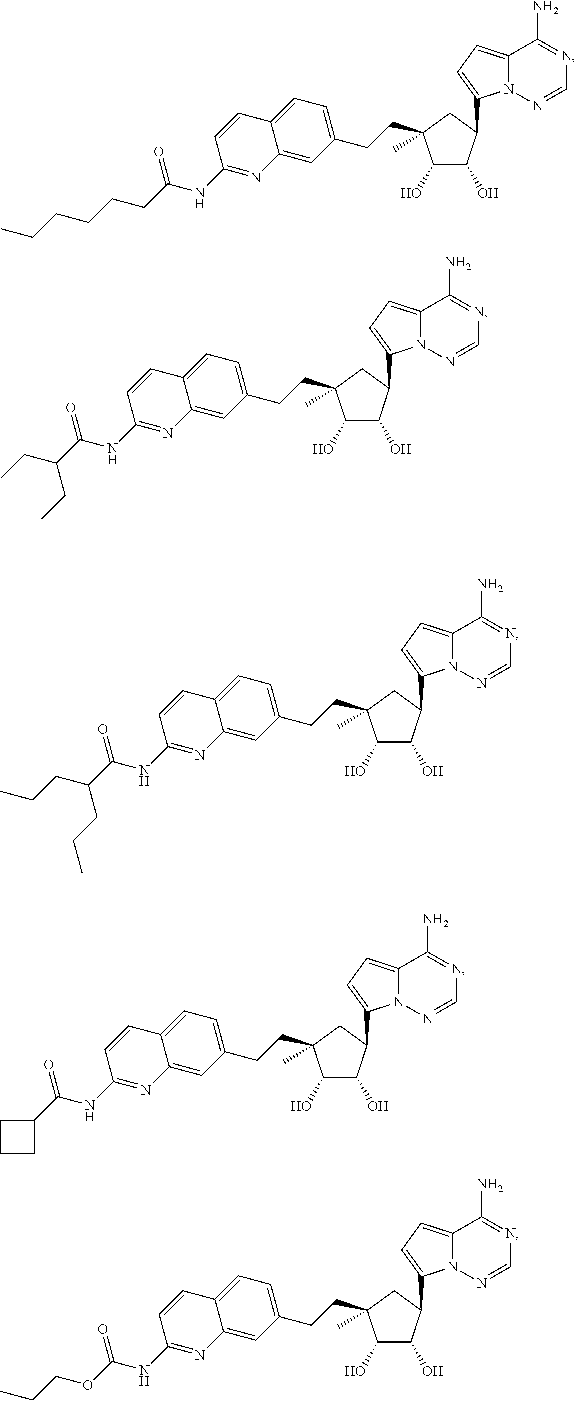 [SMILES: CCCCCCC(=O)Nc1ccc2ccc(CC[C@@]3(C)C[C@@H](c4ccc5c(N)ncnn45)[C@H](O)[C@@H]3O)cc2n1 ; CCC(CC)C(=O)Nc1ccc2ccc(CC[C@@]3(C)C[C@@H](c4ccc5c(N)ncnn45)[C@H](O)[C@@H]3O)cc2n1 ; CCCC(CCC)C(=O)Nc1ccc2ccc(CC[C@@]3(C)C[C@@H](c4ccc5c(N)ncnn45)[C@H](O)[C@@H]3O)cc2n1 ; C[C@]1(CCc2ccc3ccc(NC(=O)C4CCC4)nc3c2)C[C@@H](c2ccc3c(N)ncnn23)[C@H](O)[C@@H]1O ; CCCOC(=O)Nc1ccc2ccc(CC[C@@]3(C)C[C@@H](c4ccc5c(N)ncnn45)[C@H](O)[C@@H]3O)cc2n1] 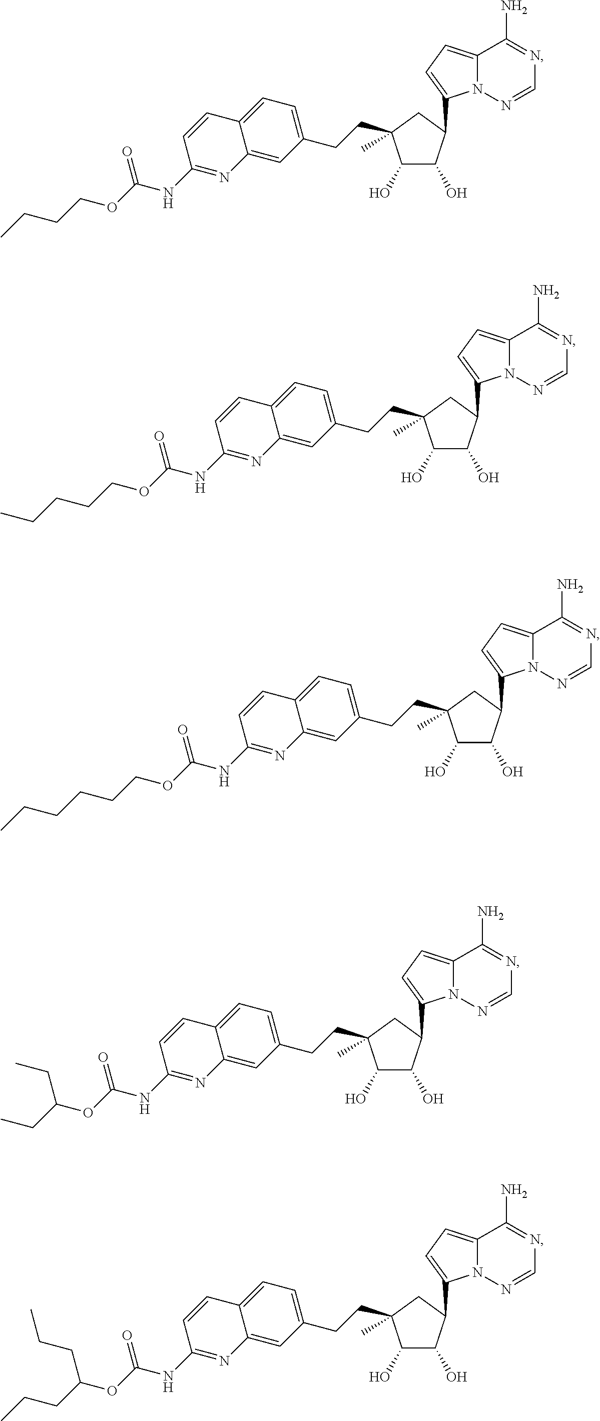 [SMILES: CCCCOC(=O)Nc1ccc2ccc(CC[C@@]3(C)C[C@@H](c4ccc5c(N)ncnn45)[C@H](O)[C@@H]3O)cc2n1 ; CCCCCOC(=O)Nc1ccc2ccc(CC[C@@]3(C)C[C@@H](c4ccc5c(N)ncnn45)[C@H](O)[C@@H]3O)cc2n1 ; CCCCCCOC(=O)Nc1ccc2ccc(CC[C@@]3(C)C[C@@H](c4ccc5c(N)ncnn45)[C@H](O)[C@@H]3O)cc2n1 ; CCC(CC)OC(=O)Nc1ccc2ccc(CC[C@@]3(C)C[C@@H](c4ccc5c(N)ncnn45)[C@H](O)[C@@H]3O)cc2n1 ; CCCC(CCC)OC(=O)Nc1ccc2ccc(CC[C@@]3(C)C[C@@H](c4ccc5c(N)ncnn45)[C@H](O)[C@@H]3O)cc2n1] 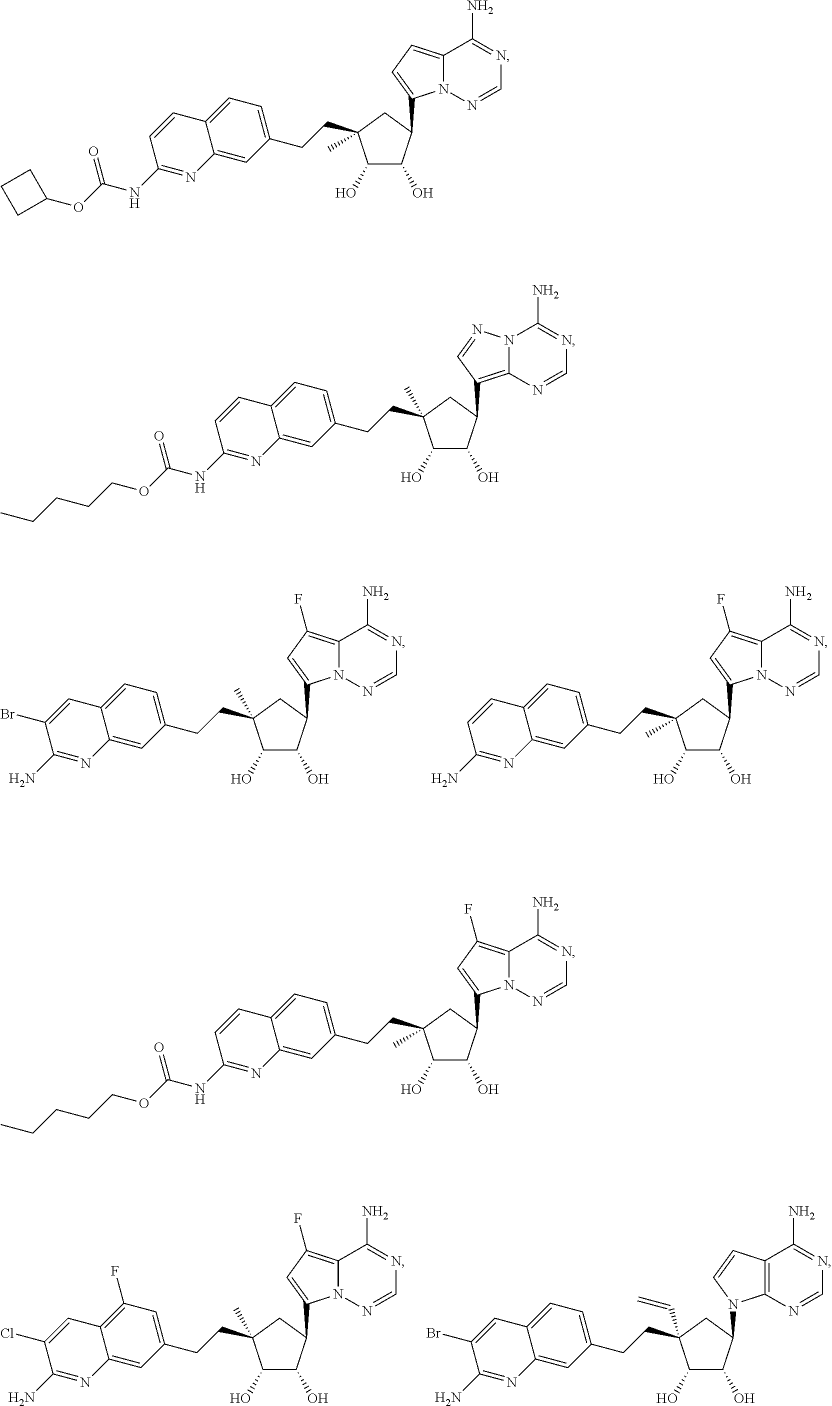 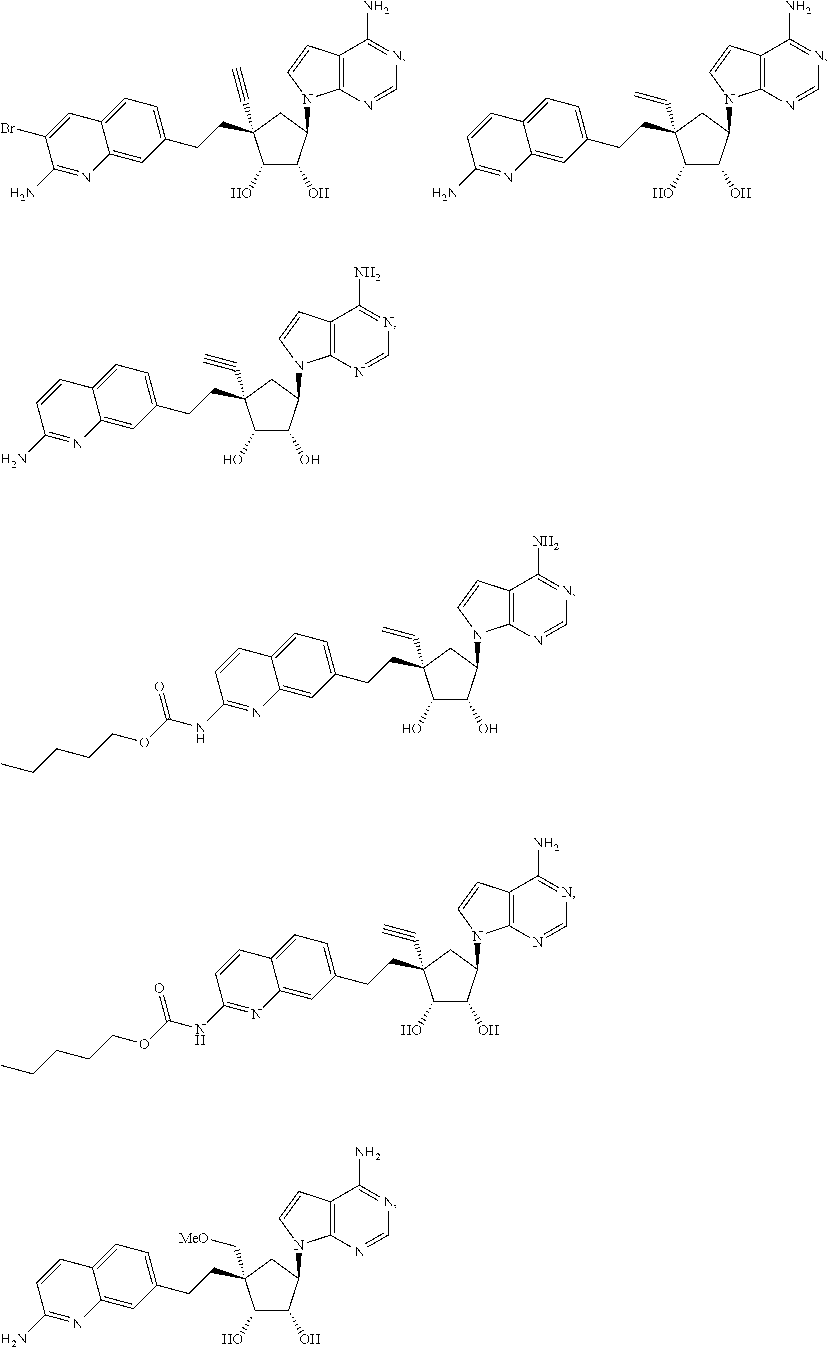 [SMILES: C#C[C@]1(CCc2ccc3cc(Br)c(N)nc3c2)C[C@@H](n2ccc3c(N)ncnc32)[C@H](O)[C@@H]1O ; C=C[C@]1(CCc2ccc3ccc(N)nc3c2)C[C@@H](n2ccc3c(N)ncnc32)[C@H](O)[C@@H]1O ; C#C[C@]1(CCc2ccc3ccc(N)nc3c2)C[C@@H](n2ccc3c(N)ncnc32)[C@H](O)[C@@H]1O ; C=C[C@]1(CCc2ccc3ccc(NC(=O)OCCCCC)nc3c2)C[C@@H](n2ccc3c(N)ncnc32)[C@H](O)[C@@H]1O ; C#C[C@]1(CCc2ccc3ccc(NC(=O)OCCCCC)nc3c2)C[C@@H](n2ccc3c(N)ncnc32)[C@H](O)[C@@H]1O ; COC[C@]1(CCc2ccc3ccc(N)nc3c2)C[C@@H](n2ccc3c(N)ncnc32)[C@H](O)[C@@H]1O] 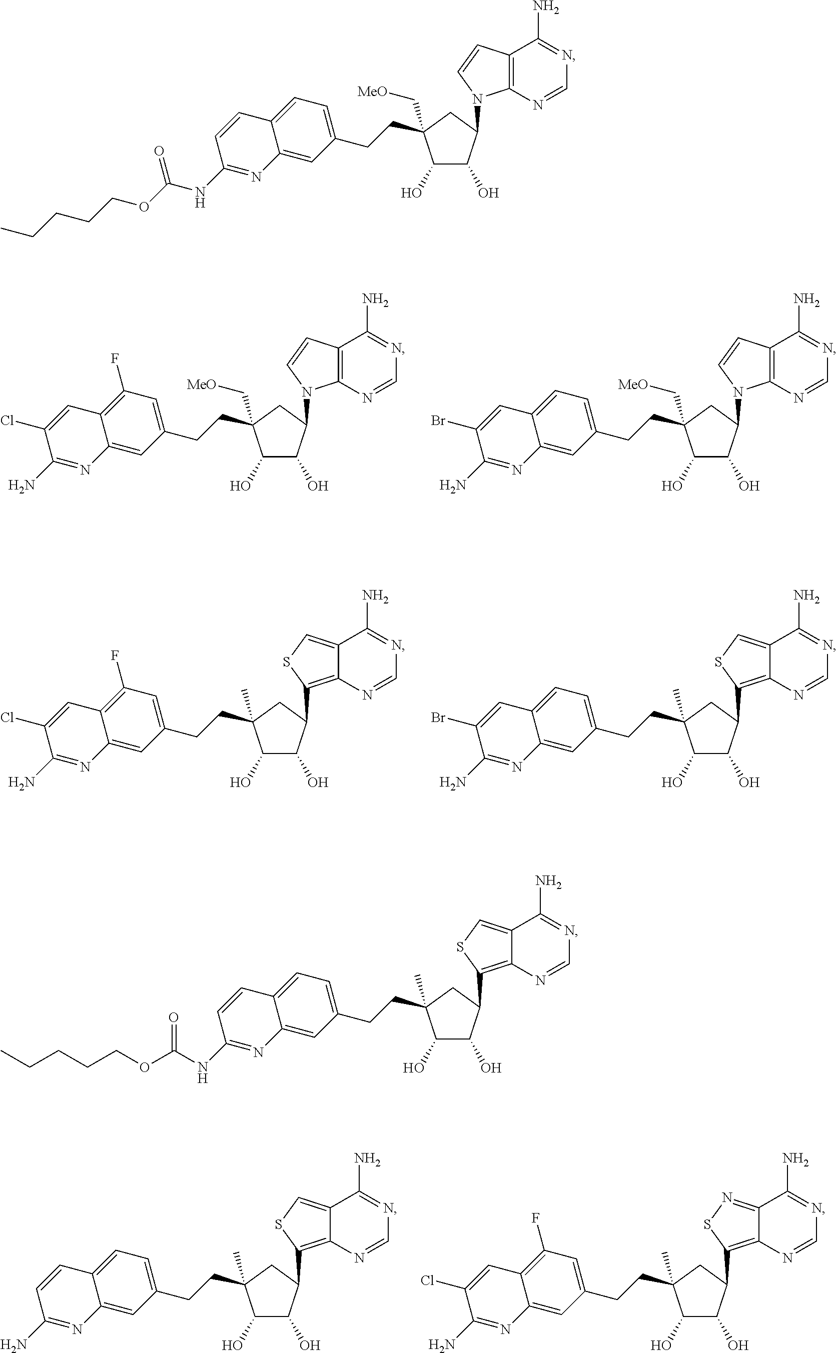 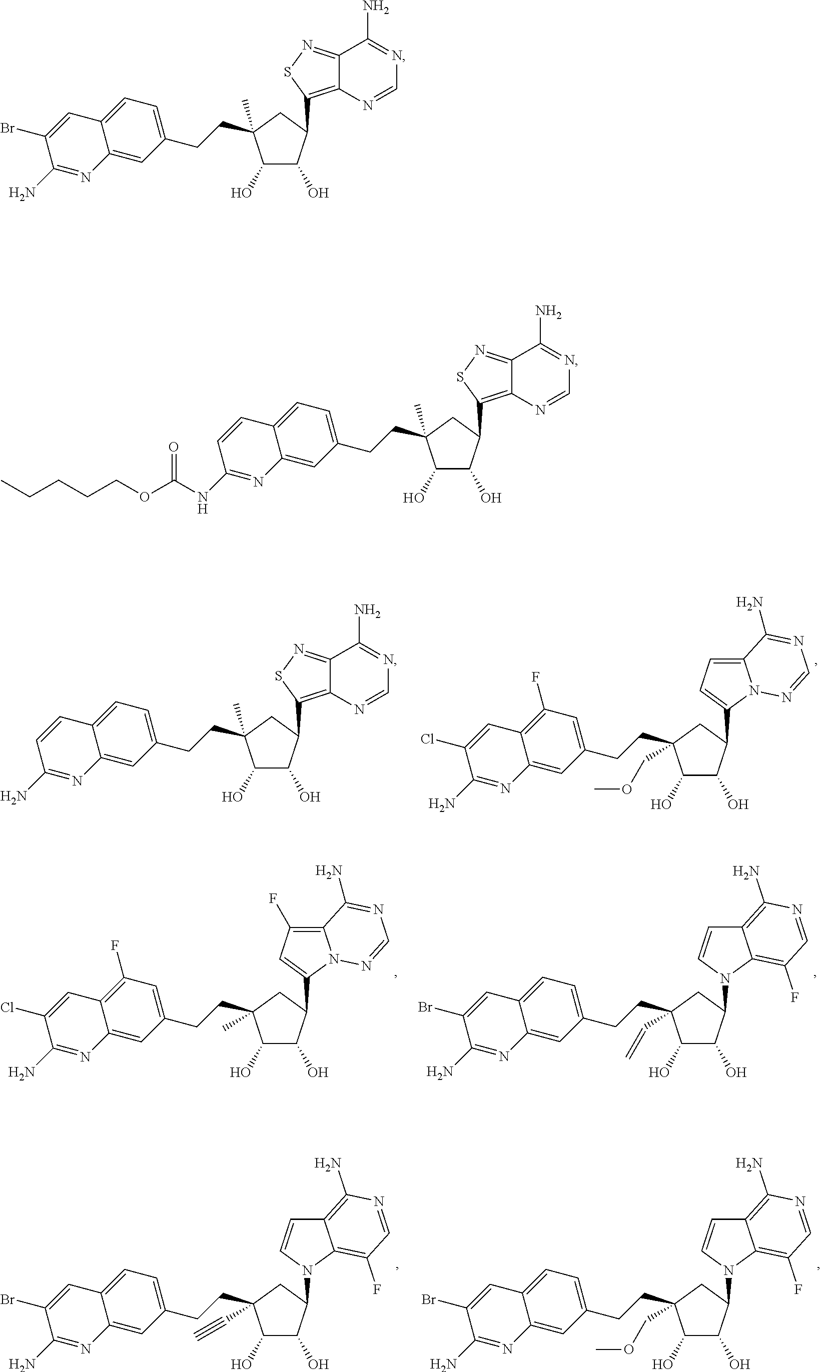 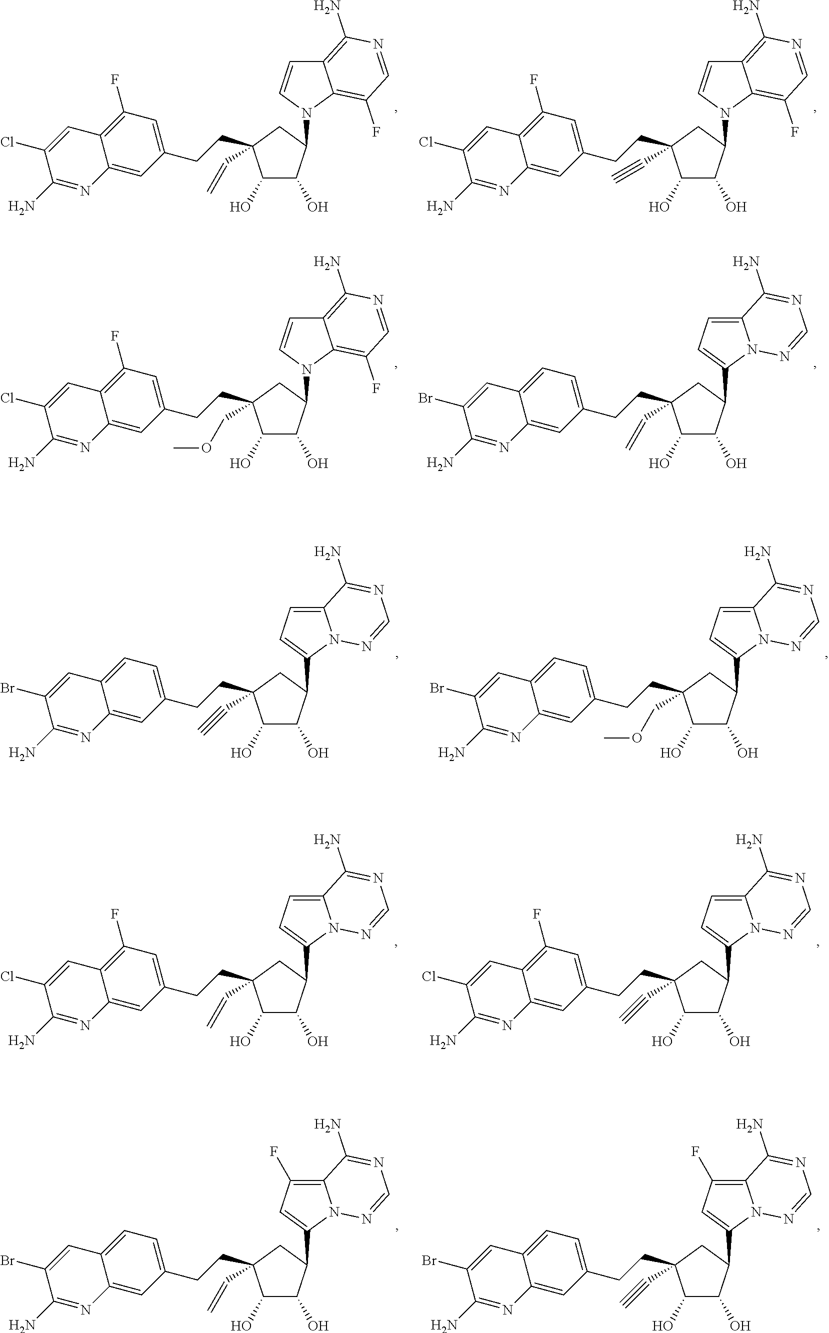 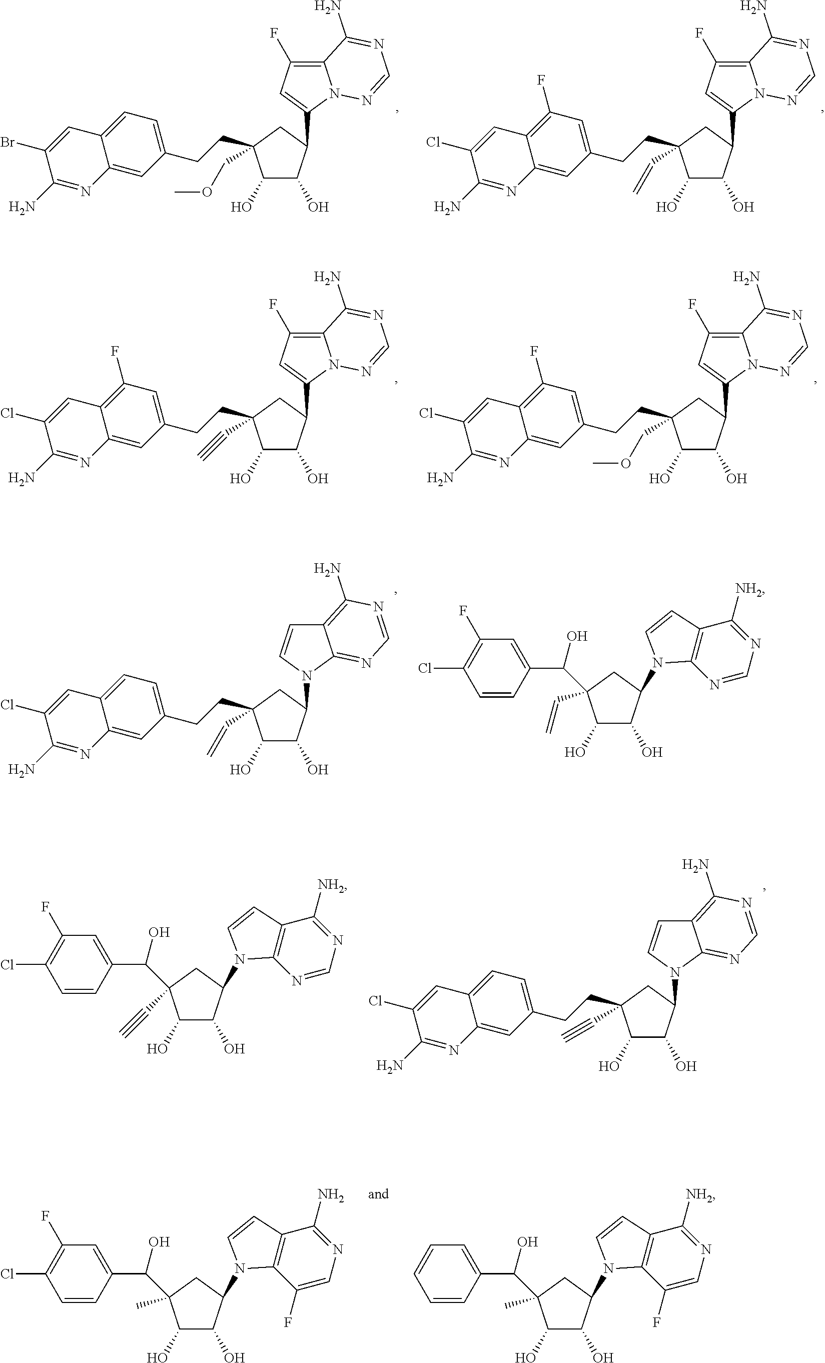
or a pharmaceutically acceptable salt of any of the foregoing.

166. (canceled)

167. A pharmaceutical composition comprising an effective amount of a compound of claim 1, or a pharmaceutically acceptable salt thereof, and excipient.

168. (canceled)

169. (canceled)

170. (canceled)

171. (canceled)

172. (canceled)

173. (canceled)

174. A method for treating a melanoma comprising administering an effective amount of a compound of claim 1, or a pharmaceutically acceptable salt thereof, to a subject in need thereof.

175. A method for inhibiting replication of a melanoma cell comprising contacting the cancer cell with an effective amount of a compound of claim 1, or a pharmaceutically acceptable salt thereof.

176. (canceled)

177. A method for treating a cancer that comprises identifying a subject suffering from a cancer and possessing wild-type (WT) p53; and administering to the identified subject an effective amount of a compound of claim 1, or a pharmaceutically acceptable salt thereof.

178. (canceled)

179. (canceled)
